(19) United States
(12) Patent Application Publication
(10) Pub. No.: US 2024/0251663 A1
(43) Pub. Date: Jul. 25, 2024

(54) LIGHT-EMITTING DEVICE INCLUDING AMINE-CONTAINING COMPOUND, ELECTRONIC APPARATUS INCLUDING THE LIGHT-EMITTING DEVICE, AND THE AMINE-CONTAINING COMPOUND

(71) Applicant: Samsung Display Co., Ltd.

(72) Inventors: Jeongmin Lee, Yongin-si (KR); Minji Kim, Yongin-si (KR); Hankyu Pak, Yongin-si (KR); Hyunbin Park, Yongin-si (KR); Byeongwook Yoo, Yongin-si (KR); Sohee Jo, Yongin-si (KR)

(21) Appl. No.: 18/236,882

(22) Filed: Aug. 22, 2023

(30) Foreign Application Priority Data
Dec. 15, 2022 (KR) 10-2022-0176244

(51) Int. Cl. H10K 85/60; C09K 11/06; H10K 50/15

(52) U.S. Cl. CPC H10K 85/633; C09K 11/06; H10K 50/15; H10K 85/615; C09K 2211/1014

(57) ABSTRACT

A light-emitting device that includes an amine-containing compound. An electronic apparatus and an electronic equipment that includes the light-emitting device. Utilization of the amine-containing compound in the light-emitting device may enhance or improve the operating characteristics of the device, such as driving voltage, luminance, efficiency, and/or lifespan.

### CROSS-REFERENCE TO RELATED APPLICATION

This application claims priority to and the benefit of Korean Patent Application No. 10-2022-0176244, filed on Dec. 15, 2022, in the Korean Intellectual Property Office, the content of which is incorporated by reference herein in its entirety.

### BACKGROUND

### 1. Field

One or more aspects of embodiments of the present disclosure relate to a light-emitting device including an amine-containing compound, an electronic apparatus including the light-emitting device, and the amine-containing compound.

### 2. Description of the Related Art

From among light-emitting devices, self-emissive devices have wide viewing angles, high contrast ratios, short response times, and excellent or suitable characteristics in terms of luminance, driving voltage, and response speed.

In a light-emitting device, a first electrode is located on a substrate, and a hole transport region, an emission layer, an electron transport region, and a second electrode are sequentially arranged on the first electrode. Holes provided from the first electrode move toward the emission layer through the hole transport region, and electrons provided from the second electrode move toward the emission layer through the electron transport region. Carriers, such as holes and electrons, recombine in the emission layer to produce excitons. These excitons transition (relax) from an excited state to a ground state to thereby generate light.

### SUMMARY

One or more aspects of embodiments of the present disclosure are directed toward a light-emitting device including an amine-containing compound, an electronic apparatus including the light-emitting device, and the amine-containing compound.

Additional aspects will be set forth in part in the description which follows and, in part, will be apparent from the description, or may be learned by practice of the presented embodiments of the disclosure.

One or more embodiments of the present disclosure relate to a light-emitting device that includes:
a first electrode,
a second electrode facing the first electrode,
an interlayer located between the first electrode and the second electrode and including an emission layer, and
an amine-containing compound represented by Formula 1 or 2.

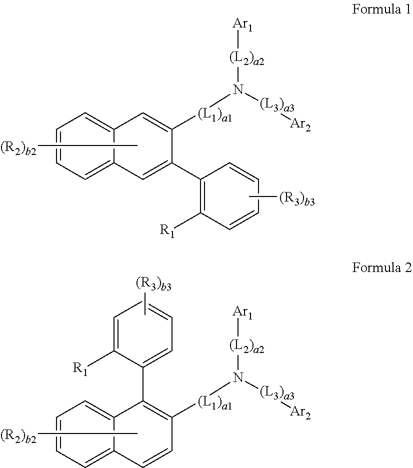

In Formulae 1 and 2,
L₁ to L₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
Ar₁ and Ar₂ may each independently be a C₆-C₆₀ aryl group unsubstituted or substituted with at least one R₁₀ₐ or a C₁-C₆₀ heteroaryl group unsubstituted or substituted with at least one R₁₀ₐ,
R₁ may be deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
R₂ and R₃ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
a1 to a3, b2, and b3 may each independently be an integer from 0 to 6,
when b3 is 1 or more and R₃ is not hydrogen, R₁ and R₃ may not be linked to each other,
R₁₀ₐ may be
deuterium, —F, —Cl, —Br, —I, a hydroxyl group, a cyano group, or a nitro group,
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or a combination thereof,
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or a combination thereof, or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
wherein Q₁ to Q₃, Q₁₁ to Q₁₃, Q₂₁ to Q₂₃, and Q₃₁ to Q₃₃ may each independently be hydrogen, deuterium, —F, —Cl, —Br, —I, a hydroxyl group, a cyano group, a nitro group, or a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or a combination thereof.

One or more embodiments of the present disclosure relate to an electronic apparatus and electronic equipment that include the light-emitting device.

One or more embodiments of the present disclosure relate to the amine-containing compound represented by Formula 1 or 2.

### BRIEF DESCRIPTION OF THE DRAWINGS

The above and other aspects will be more apparent and more readily appreciated from the following description of the embodiments, taken in conjunction with the accompanying drawings, in which:

FIG. 1 is a schematic view of a light-emitting device according to an embodiment;

FIG. 2 is a schematic cross-sectional view of an electronic apparatus according to an embodiment;

FIG. 3 is a schematic cross-sectional view of an electronic apparatus according to an embodiment;

FIG. 4 is a schematic perspective view of an electronic equipment according to an embodiment;

FIG. 5 is a schematic perspective view of the exterior of a vehicle;

FIG. 6A is a schematic view of the interior of a vehicle that includes an electronic equipment according to an embodiment;

FIG. 6B is a schematic view of the interior of a vehicle that includes an electronic equipment according to an embodiment; and

FIG. 6C is a schematic view of the interior of a vehicle that includes an electronic equipment according to an embodiment.

### DETAILED DESCRIPTION

Reference will now be made in more detail to embodiments of the present disclosure, examples of which are illustrated in the accompanying drawings, wherein like reference numerals refer to like elements throughout, and duplicative descriptions thereof may not be provided. In this regard, the present embodiments may have different forms and should not be construed as being limited to the descriptions set forth herein. Accordingly, the embodiments are merely described, by referring to the drawings, to explain aspects of the present description.

As utilized herein, the term “and/or” includes any and all combinations of one or more of the associated listed items. As used herein, expressions such as “at least one of,” “one of,” and “selected from,” when preceding a list of elements, modify the entire list of elements and do not modify the individual elements of the list. For example, the expressions “at least one of a to c,” “at least one of a, b or c,” and “at least one of a, b and/or c” may indicate only a, only b, only c, both (e.g., simultaneously) a and b, both (e.g., simultaneously) a and c, both (e.g., simultaneously) b and c, all of a, b, and c, or variations thereof.

The terminology used herein is for the purpose of describing embodiments and is not intended to limit the embodiments described herein. Unless otherwise defined, all chemical names, technical and scientific terms, and terms defined in common dictionaries should be interpreted as having meanings consistent with the context of the related art, and should not be interpreted in an ideal or overly formal sense. It will be understood that, although the terms first, second, etc. may be used herein to describe various elements, these elements should not be limited by these terms. These terms are only used to distinguish one element from another element. Thus, a first element could be termed a second element without departing from the teachings of the present disclosure. Similarly, a second element could be termed a first element.

As used herein, singular forms such as “a,” “an,” and “the” are intended to include the plural forms as well, unless the context clearly indicates otherwise.

It will be further understood that the terms “includes,” “including,” “comprises,” and/or “comprising,” when used in this specification, specify the presence of stated features, steps, operations, elements, and/or components, but do not preclude the presence or addition of one or more other features, steps, operations, elements, components, and/or groups thereof.

As used herein, the terms “use,” “using,” and “used” may be considered synonymous with the terms “utilize,” “utilizing,” and “utilized,” respectively.

As used herein, the term “and/or” includes any, and all, combination(s) of one or more of the associated listed items.

The term “may” will be understood to refer to “one or more embodiments of the present disclosure,” some of which include the described element and some of which exclude that element and/or include an alternate element. Similarly, alternative language such as “or” refers to “one or more embodiments of the present disclosure,” each including a corresponding listed item.

It will be understood that when an element is referred to as being “on,” “connected to,” or “on” another element, it may be directly on, connected, or coupled to the other element or one or more intervening elements may also be present. When an element is referred to as being “directly on,” “directly connected to,” or “directly coupled to” another element, there are no intervening elements present.

Spatially relative terms, such as “beneath,” “below,” “lower,” “above,” “upper,” “bottom,” “top,” and the like, may be used herein for ease of description to describe one element or feature's relationship to another element(s) or feature(s) as illustrated in the drawings. It will be understood that the spatially relative terms are intended to encompass different orientations of the device in use or operation in addition to the orientation depicted in the drawings. For example, if the device in the drawings is turned over, elements described as “below” or “beneath” other elements or features would then be oriented “above” or “over” the other elements or features. Thus, the term “below” may encompass both an orientation of above and below. The device may be otherwise oriented (rotated 90 degrees or at other orientations), and the spatially relative descriptors used herein should be interpreted accordingly.

Unless otherwise defined, all terms (including technical and scientific terms) used herein have the same meaning as commonly understood by those of ordinary skill in the art to which the present disclosure pertains. It is also to be understood that terms defined in commonly used dictionaries should be interpreted as having meanings consistent with meanings in the context of the related art, unless expressly defined herein, and should not be interpreted in an ideal or overly formal sense.

In this context, “consisting essentially of” means that any additional components will not materially affect the chemical, physical, optical or electrical properties of the semiconductor film.

Further, in this specification, the phrase “on a plane,” or “plan view,” means viewing a target portion from the top, and the phrase “on a cross-section” means viewing a cross-section formed by vertically cutting a target portion from the side.

Aspects of one or more embodiments of the present disclosure relate to a light-emitting device that may includes a first electrode, a second electrode, an interlayer, and an amine-containing compound. The second electrode is facing, or is configured toward, the first electrode. In an embodiment, components of the second electrode are facing, or are configured toward, components of the first electrode. In some embodiments, the interlayer may be arranged between the first electrode and the second electrode. In some embodiments, the interlayer may include an emission layer.

In several embodiments, the amine-containing compound is represented by Formula 1 or 2:

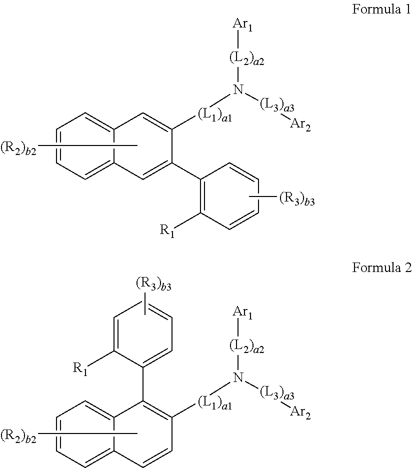

wherein, in Formula 1 or 2, L₁, L₂, and L₃ may each independently be a C₅-C₆₀ carbocyclic group unsubstituted or substituted with at least one R₁₀ₐ or a C₁-C₆₀ heterocyclic group unsubstituted or substituted with at least one R₁₀ₐ.

In an embodiment, L₁, L₂, and L₃ may each independently be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, an indazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, or an azadibenzofuran group, each unsubstituted or substituted with at least one R₁₀ₐ.

In an embodiment, L₁, L₂, and L₃ may each independently be a cyclopentane group, a cyclohexane group, a benzene group, a naphthalene group, an azulene group, an indacene group, an acenaphthylene group, a phenalene group, a phenanthrene group, an anthracene group, a pyrrole group, a thiophene group, a furan group, an indole group, a benzoindole group, a naphtho indole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, a pyrazole group, an imidazole group, a triazole group, a benzopyrazole group, a benzimidazole group, an indazole group, a pyridine group, a pyrimidine group, a pyrazine group, a pyridazine group, a triazine group, a quinoline group, an isoquinoline group, a benzoquinoline group, a benzoisoquinoline group, an imidazopyridine group, an imidazopyrimidine group, an imidazotriazine group, an imidazopyrazine group, an imidazopyridazine group, an azacarbazole group, an azadibenzosilole group, an azadibenzothiophene group, or an azadibenzofuran group, each unsubstituted or substituted with at least one deuterium, a C₁-C₂₀ alkyl group, a cyclohexane group, an adamantane group, a norbornane group, a benzene group, a naphthalene group, a phenanthrene group, a fluorene group, spiro-bifluorene group, a dibenzofuran group, a dibenzothiophene group, or a combination thereof.

In an embodiment, L₁, L₂, and L₃ may each independently be one of

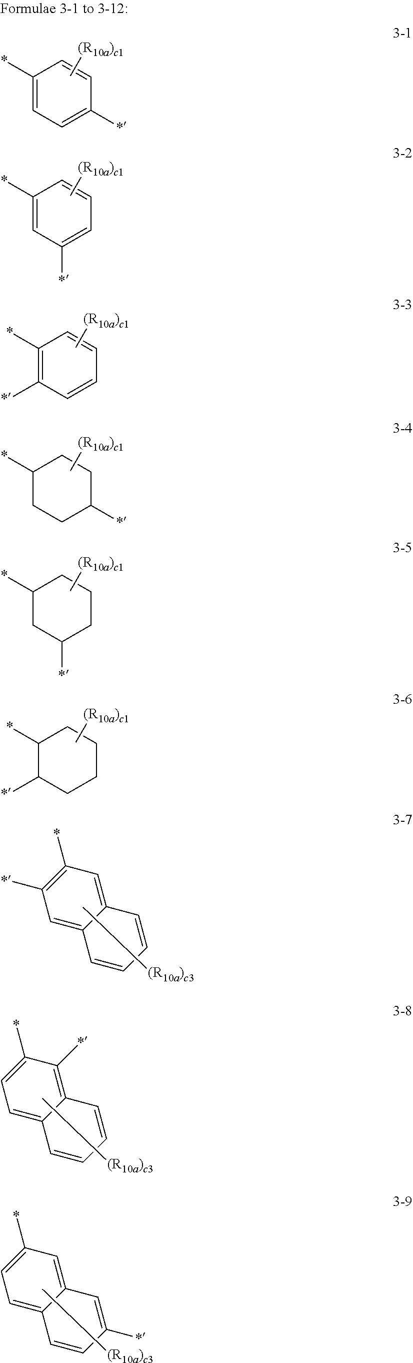

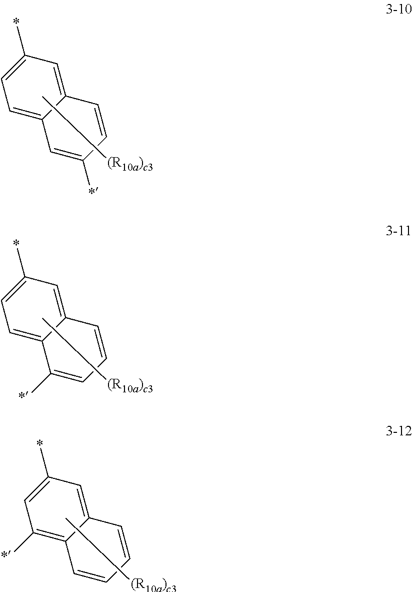

wherein, in Formulae 3-1 to 3-12,
R₁₀ₐ may be as described elsewhere herein,
c1 may be an integer from 0 to 4,
c2 may be an integer from 0 to 8,
c3 may be an integer from 0 to 6, and
* and * each indicate a binding site to a neighboring atom (i.e., a point of attachment).

In Formula 1 or 2, Ar₁ and Ar₂ may each independently be a C₆-C₆₀ aryl group unsubstituted or substituted with at least one R₁₀ₐ or a C₁-C₆₀ heteroaryl group unsubstituted or substituted with at least one R₁₀ₐ.

In an embodiment, Ar₁ and Ar₂ may each independently be a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, an indenoanthracene group, a pyrrole group, a silole group, a borole group, a 2H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, an indazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, or an azadibenzofuran group.

In an embodiment, Ar₁ and Ar₂ may each independently be a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, an indenoanthracene group, a pyrrole, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, a pyrazole group, an imidazole group, a triazole group, a benzopyrazole group, a benzimidazole group, an indazole group, a pyridine group, a pyrimidine group, a pyrazine group, a pyridazine group, a triazine group, a quinoline group, an isoquinoline group, a benzoquinoline group, a benzoisoquinoline group, an imidazopyridine group, an imidazopyrimidine group, an imidazotriazine group, an imidazopyrazine group, an imidazopyridazine group, an azacarbazole group, an azadibenzosilole group, an azadibenzothiophene group, or an azadibenzofuran group.

In an embodiment, Ar₁ and Ar₂ may each independently be a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, or a fluorene group, each unsubstituted or substituted with at least one deuterium, a C₁-C₂₀ alkyl group, a cyclohexane group, an adamantane group, a norbornane group, a benzene group, a naphthalene group, a phenanthrene group, a fluorene group, a spiro-bifluorene group, a dibenzofuran group, a dibenzothiophene group, or a combination thereof.

In an embodiment, Ar₁ and Ar₂ may each independently be one of Formulae 4-1 to 4-13:

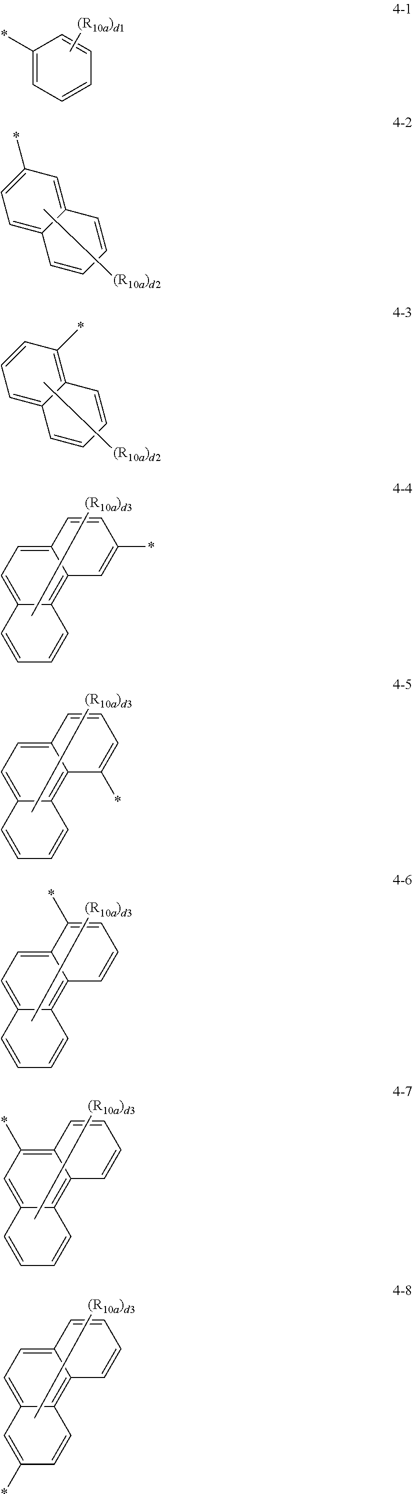

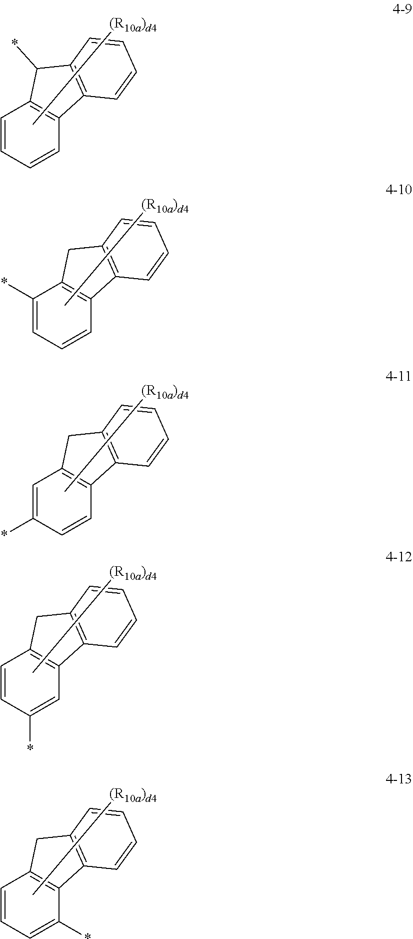

wherein, in Formulae 4-1 to 4-13,
R₁₀ₐ may be as described elsewhere herein,
d1 may be an integer from 0 to 5,
d2 may be an integer from 0 to 7,
d3 may be an integer from 0 to 10,
d4 may be an integer from 0 to 10, and
* indicates a binding site to a neighboring atom (i.e., a point of attachment).

In Formula 1 or 2, R₁ may be deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁) or —P(═O)(Q₁)(Q₂). In an embodiment, Q₁, Q₂, and Q₃ may each independently be: hydrogen; deuterium; —F; —Cl; —Br; —I; a hydroxyl group; a cyano group; a nitro group; or a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, or a C₁-C₆₀ heterocyclic group. In an embodiment, each of C₁-C₆₀ alkyl group, C₂-C₆₀ alkenyl group, C₂-C₆₀ alkynyl group, C₁-C₆₀ alkoxy group, C₃-C₆₀ carbocyclic group, and/or C₁-C₆₀ heterocyclic group independently is unsubstituted or substituted with deuterium, —F, a cyano group, a C₁-C₆₀ alkyl group, a C₁-C₆₀ alkoxy group, a phenyl group, a biphenyl group, or a combination thereof.

For example, R₁ may be deuterium, —F, or a cyano group; or a C₁-C₂₀ alkyl group unsubstituted or substituted with hydrogen, deuterium, —F, a cyano group, or a combination thereof.

In an embodiment, R₁ may be a methyl group, an ethyl group, an n-propyl group, an isopropyl group, an n-butyl group, an isobutyl group, a sec-butyl group, a tert-butyl group, an n-pentyl group, an isopentyl group, a sec-pentyl group, a tert-pentyl group, a 2-methylbutyl group, a 2-2dimethylpropyl group, a 1-ethylpropyl group, or a 1,2-dimethylpropyl group, each unsubstituted or substituted with deuterium, —F, —Cl, —Br, —I, a hydroxyl group, a cyano group, or a nitro group.

In Formula 1 or 2, R₂ and R₃ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂). In an embodiment, Q₁ to Q₃ may each independently be: hydrogen; deuterium; —F; —Cl; —Br; —I; a hydroxyl group; a cyano group; a nitro group; or a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, or a C₁-C₆₀ heterocyclic group. In an embodiment, each of C₁-C₆₀ alkyl group, C₂-C₆₀ alkenyl group, C₂-C₆₀ alkynyl group, C₁-C₆₀ alkoxy group, C₃-C₆₀ carbocyclic group, and/or C₁-C₆₀ heterocyclic group independently is unsubstituted or substituted with deuterium, —F, a cyano group, a C₁-C₆₀ alkyl group, a C₁-C₆₀ alkoxy group, a phenyl group, a biphenyl group, or a combination thereof.

For example, R₂ and R₃ may each independently be hydrogen, deuterium, —F, a cyano group, a C₁-C₂₀ alkyl group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ.

For example, R₂ and R₃ may each independently be: hydrogen, deuterium, —F, or a cyano group; or
a C₁-C₂₀ alkyl group unsubstituted or substituted with hydrogen, deuterium, —F, a cyano group, or a combination thereof; or
a benzene group, a pyridine group, a dibenzofuran group, a dibenzothiophene group, a carbazole group, an azadibenzofuran group, an azadibenzothiophene group, or an azacarbazole group, each unsubstituted or substituted with hydrogen, deuterium, —F, a cyano group, a C₁-C₂₀ alkyl group, a deuterated C₁-C₂₀ alkyl group, a fluorinated C₁-C₂₀ alkyl group, a phenyl group, a deuterated phenyl group, a fluorinated phenyl group, a (C₁-C₂₀ alkyl) phenyl group, a biphenyl group, a dibenzofuranyl group, a dibenzothiophenyl group, a carbazolyl group, a fluorenyl group, a dibenzosilolyl group, or a combination thereof.

In an embodiment, R₂ and R₃ may each independently be: hydrogen, deuterium, —F, —Cl, —Br, —I, a cyano group, a C₁-C₂₀ alkyl group; or
a C₁-C₂₀ alkyl group substituted with at least one of deuterium, —F, —Cl, —Br, —I, —CD₃, —CD₂H, —CDH₂, —CF₃, —CF₂H, —CFH₂, a cyano group, a nitro group, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a naphthyl group, a pyridinyl group, and a pyrimidinyl group; or
a phenyl group, a pyridine group, a dibenzofuranyl group, a dibenzothiophenyl group, a carbazolyl group, an azadibenzofuranyl group, an azadibenzothiophenyl group, or an aza carbazolyl group, each unsubstituted or substituted with at least one of deuterium, —F, —Cl, —Br, —I, —CD₃, —CD₂H, —CDH₂, —CF₃, —CF₂H, —CFH₂, a cyano group, a C₁-C₂₀ alkyl group, a phenyl group, a biphenyl group, a C₁-C₁₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzoimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, —Si(Q₃₁)(Q₃₂)(Q₃₃), or —N(Q₃₁)(Q₃₂), —B(Q₃₁)(Q₃₂), or
—Si(Q₁)(Q₂)(Q₃), —N(Q₁)(Q₂), or —B(Q₁)(Q₂), and
Q₁ to Q₃ and Q₃₁ to Q₃₃ may each independently be:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂; or
an n-propyl group, an iso-propyl group, an n-butyl group, an isobutyl group, a sec-butyl group, a tert-butyl group, an n-pentyl group, an isopentyl group, a sec-pentyl group, a tert-pentyl group, a phenyl group, a naphthyl group, a pyridinyl group, a pyrimidinyl group, a pyridazinyl group, a pyrazinyl group, or a triazinyl group, each unsubstituted or substituted with at least one of deuterium, a C₁-C₁₀ alkyl group, a phenyl group, a biphenyl group, a pyridinyl group, a pyrimidinyl group, a pyridazinyl group, a pyrazinyl group, or a triazinyl group.

In an embodiment, R₂ and R₃ may each independently be:
a methyl group, an ethyl group, an n-propyl group, an iso-propyl group, an n-butyl group, an isobutyl group, a sec-butyl group, a tert-butyl group, an n-pentyl group, an isopentyl group, a sec-pentyl group, a tert-pentyl group, a 2-methylbutyl group, a 2-2dimethylpropyl group, a 1-ethylpropyl group, or a 1,2-dimethylpropyl group, each unsubstituted or substituted with deuterium, —F, —Cl, —Br, —I, a hydroxyl group, a cyano group, or a nitro group; or
a benzene group, a pyridine group, a dibenzofuran group, a dibenzothiophene group, a carbazole group, an azadibenzofuran group, an azadibenzothiophene group, or an azacarbazole group, each unsubstituted or substituted with a methyl group, an ethyl group, an n-propyl group, an isopropyl group, an n-butyl group, an isobutyl group, a sec-butyl group, a tert-butyl group, an n-pentyl group, an isopentyl group, a sec-pentyl group, a tert-pentyl group, a 2-methylbutyl group, a 2-2dimethylpropyl group, a 1-ethylpropyl group, a 1,2-dimethylpropyl group, or a phenyl group, each unsubstituted or substituted with deuterium, —F, —Cl, —Br, —I, a hydroxyl group, a cyano group, or a nitro group.

In Formula 1 or 2, a1, a2, a3, b2, and b3 may each independently be an integer from 0 to 6.

In an embodiment, a1, ar, and a3 may each independently be an integer from 0 to 2.

In an embodiment, b2 and b3 may each independently be an integer from 0 to 3.

In Formula 1 or 2, when is b3 is 1 or more and R₃ is not hydrogen, R₁ and R₃ may not be linked to each other. For example, when b3 is 1 or more and R₃ is not hydrogen, R₁ and R₃ may not be linked to each other to form a C₅-C₃₀ carbocyclic group or a C₁-C₃₀ heterocyclic group.

In Formula 1 or 2, each R₁₀ₐ independently may be:
deuterium, —F, —Cl, —Br, —I, a hydroxyl group, a cyano group, or a nitro group; or
a C₁-C₆₀ alkyl group, a C₂-C₆₀ alkenyl group, a C₂-C₆₀ alkynyl group, or a C₁-C₆₀ alkoxy group. In an embodiment, each of C₁-C₆₀ alkyl group, C₂-C₆₀ alkenyl group, C₂-C₆₀ alkynyl group, and/or C₁-C₆₀ alkoxy group independently may be unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or a combination thereof; or
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group. In an embodiment, each of C₃-C₆₀ carbocyclic group, C₁-C₆₀ heterocyclic group, a C₆-C₆₀ aryloxy group, C₆-C₆₀ arylthio group, C₇-C₆₀ arylalkyl group, and/or C₂-C₆₀ heteroarylalkyl group independently may be unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or a combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

In an embodiment, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; or a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, or a C₁-C₆₀ heterocyclic group. In an embodiment, each of C₁-C₆₀ alkyl group, a C₂-C₆₀ alkenyl group, a C₂-C₆₀ alkynyl group, a C₁-C₆₀ alkoxy group, a C₃-C₆₀ carbocyclic group, and/or C₁-C₆₀ heterocyclic group may be unsubstituted or substituted with deuterium, —F, a cyano group, a C₁-C₆₀ alkyl group, a C₁-C₆₀ alkoxy group, a phenyl group, a biphenyl group, or a combination thereof.

For example, R₁₀ₐ may be:
deuterium, —F, —Cl, —Br, —I, a hydroxyl group, a cyano group, or a nitro group; or
a C₁-C₆₀ alkyl group unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or a combination thereof; or
a C₃-C₆₀ carbocyclic group unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or a combination thereof.

In an embodiment, Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; or a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, or a C₁-C₆₀ heterocyclic group. In an embodiment, each of C₁-C₆₀ alkyl group, a C₂-C₆₀ alkenyl group, a C₂-C₆₀ alkynyl group, a C₁-C₆₀ alkoxy group, a C₃-C₆₀ carbocyclic group, and/or C₁-C₆₀ heterocyclic group may be unsubstituted or substituted with deuterium, —F, a cyano group, a C₁-C₆₀ alkyl group, a C₁-C₆₀ alkoxy group, a phenyl group, a biphenyl group, or a combination thereof.

In an embodiment, R₁₀ₐ may be: a methyl group, an ethyl group, an n-propyl group, an isopropyl group, an n-butyl group, an isobutyl group, a sec-butyl group, a tert-butyl group, an n-pentyl group, an isopentyl group, a sec-pentyl group, a tert-pentyl group, a 2-methylbutyl group, a 2-2 dimethylpropyl group, a 1-ethylpropyl group, or a 1,2-dimethylpropyl group, each unsubstituted or substituted with deuterium, —F, a cyano group, or a combination thereof; or
a cyclohexane group, an adamantane group, a norbornane group, or a benzene group, each unsubstituted or substituted with a methyl group, an ethyl group, an n-propyl group, an isopropyl group, an n-butyl group, an isobutyl group, a sec-butyl group, a tert-butyl group, an n-pentyl group, an isopentyl group, a sec-pentyl group, a tert-pentyl group, a 2-methylbutyl group, a 2-2 dimethylpropyl group, a 1-ethylpropyl group, a 1,2-dimethylpropyl group, or a phenyl group, each unsubstituted or substituted with deuterium, —F, —Cl, —Br, —I, a hydroxyl group, a cyano group, or a nitro group.

In Formula 1 or 2, groups represented by *-(L₂)ₐ₂-Ar₁ and *-(L₃)ₐ₃-Ar₂ may be identical to each other. In some embodiments, groups represented by *-(L₂)ₐ₂-Ar₁ and *-(L₃)ₐ₃-Ar₂ may be different from each other.

In some embodiments, the amine-containing compound may be one selected from the groups represented by Formulae 1-1 to 1-3 and 2-1 to 2-3:

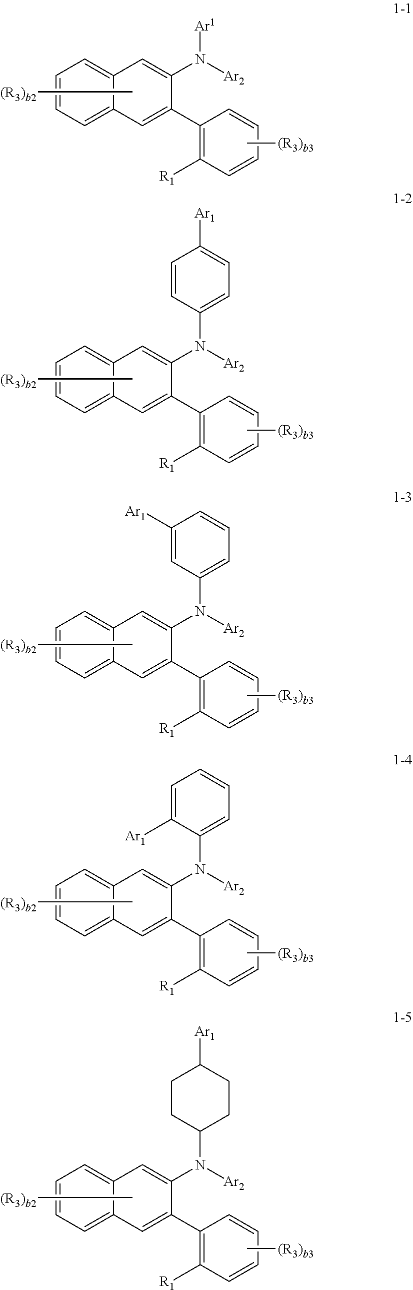

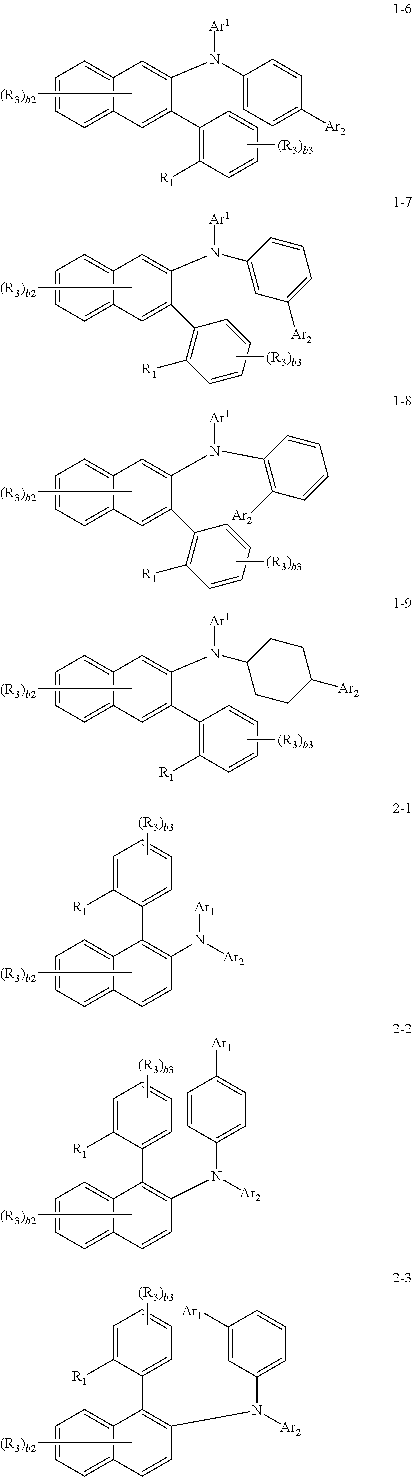

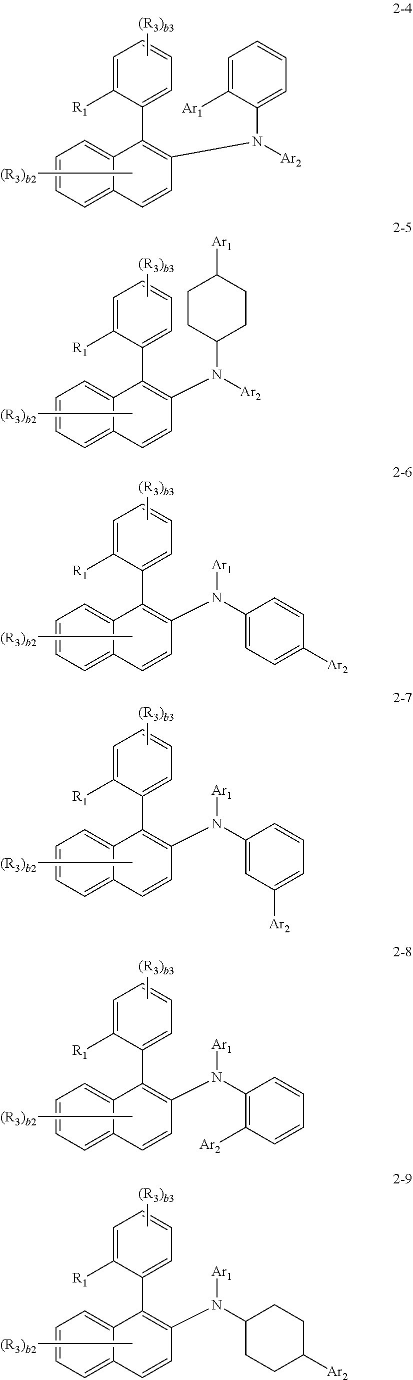

wherein, in Formulae 1-1 to 1-9 and 2-1 to 2-9,
Ar₁, Ar₂, R₁ to R₃, b2, and b3 are each as described elsewhere herein.

In some embodiments, the amine-containing compound represented by Formula 1 or 2 may be one or Compounds 1 to 385:

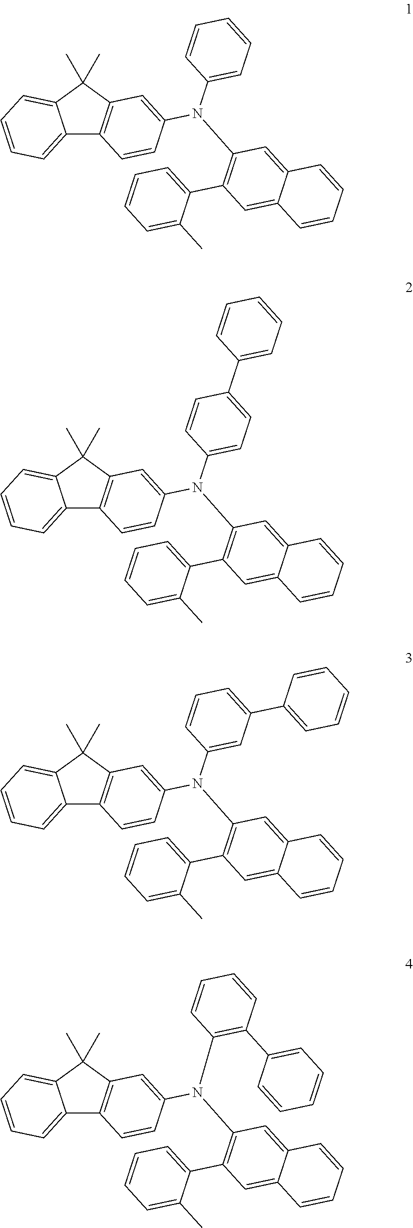 [SMILES: Cc1ccccc1-c1cc2ccccc2cc1N(c1ccccc1)c1ccc2c(c1)C(C)(C)c1ccccc1-2 ; Cc1ccccc1-c1cc2ccccc2cc1N(c1ccc(-c2ccccc2)cc1)c1ccc2c(c1)C(C)(C)c1ccccc1-2 ; Cc1ccccc1-c1cc2ccccc2cc1N(c1cccc(-c2ccccc2)c1)c1ccc2c(c1)C(C)(C)c1ccccc1-2 ; Cc1ccccc1-c1cc2ccccc2cc1N(c1ccc2c(c1)C(C)(C)c1ccccc1-2)c1ccccc1-c1ccccc1]

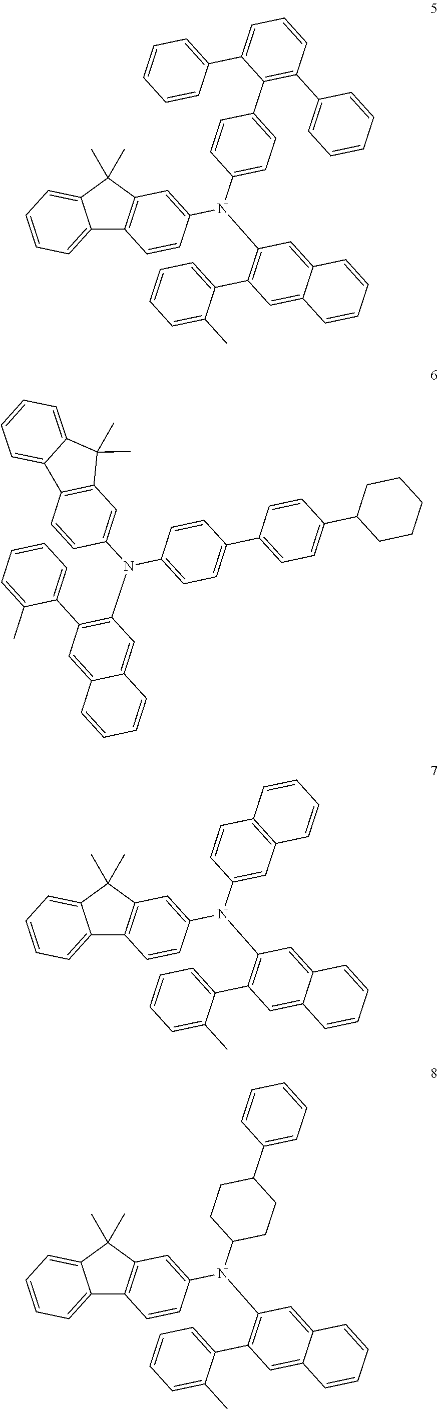 [SMILES: Cc1ccccc1-c1cc2ccccc2cc1N(c1ccc(-c2c(-c3ccccc3)cccc2-c2ccccc2)cc1)c1ccc2c(c1)C(C)(C)c1ccccc1-2 ; Cc1ccccc1-c1cc2ccccc2cc1N(c1ccc(-c2ccc(C3CCCCC3)cc2)cc1)c1ccc2c(c1)C(C)(C)c1ccccc1-2 ; Cc1ccccc1-c1cc2ccccc2cc1N(c1ccc2c(c1)C(C)(C)c1ccccc1-2)c1ccc2ccccc2c1 ; Cc1ccccc1-c1cc2ccccc2cc1N(c1ccc2c(c1)C(C)(C)c1ccccc1-2)C1CCC(c2ccccc2)CC1]

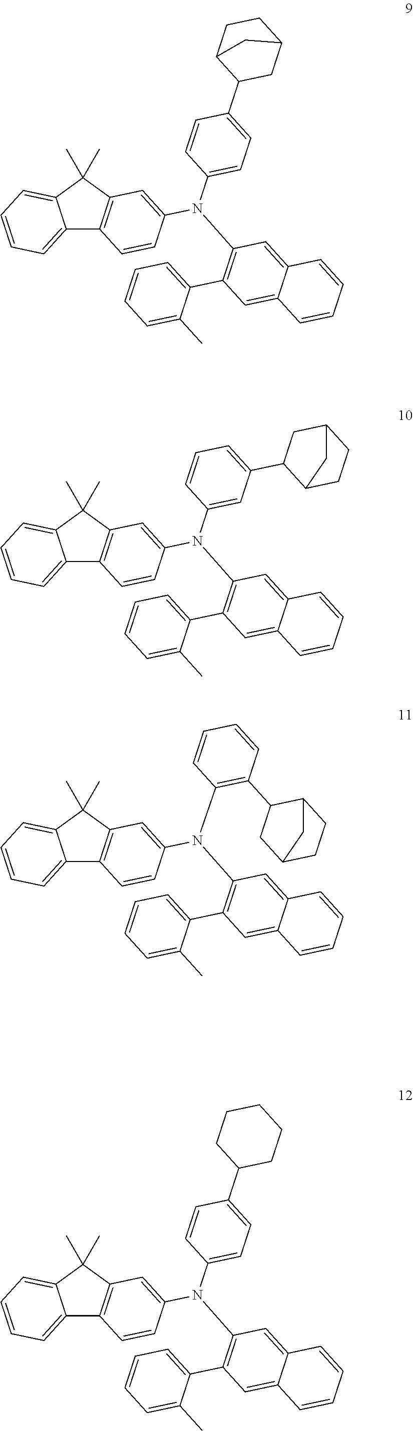 [SMILES: Cc1ccccc1-c1cc2ccccc2cc1N(c1ccc(C2CC3CCC2C3)cc1)c1ccc2c(c1)C(C)(C)c1ccccc1-2 ; Cc1ccccc1-c1cc2ccccc2cc1N(c1cccc(C2CC3CCC2C3)c1)c1ccc2c(c1)C(C)(C)c1ccccc1-2 ; Cc1ccccc1-c1cc2ccccc2cc1N(c1ccc2c(c1)C(C)(C)c1ccccc1-2)c1ccccc1C1CC2CCC1C2 ; Cc1ccccc1-c1cc2ccccc2cc1N(c1ccc(C2CCCCC2)cc1)c1ccc2c(c1)C(C)(C)c1ccccc1-2]

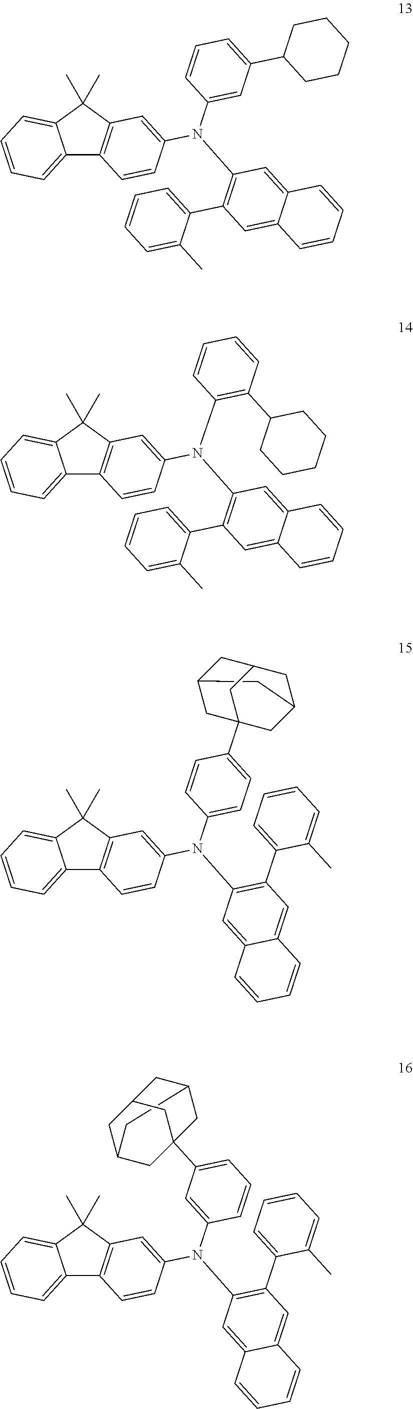 [SMILES: Cc1ccccc1-c1cc2ccccc2cc1N(c1cccc(C2CCCCC2)c1)c1ccc2c(c1)C(C)(C)c1ccccc1-2 ; Cc1ccccc1-c1cc2ccccc2cc1N(c1ccc2c(c1)C(C)(C)c1ccccc1-2)c1ccccc1C1CCCCC1 ; Cc1ccccc1-c1cc2ccccc2cc1N(c1ccc(C23CC4CC(CC(C4)C2)C3)cc1)c1ccc2c(c1)C(C)(C)c1ccccc1-2 ; Cc1ccccc1-c1cc2ccccc2cc1N(c1cccc(C23CC4CC(CC(C4)C2)C3)c1)c1ccc2c(c1)C(C)(C)c1ccccc1-2]

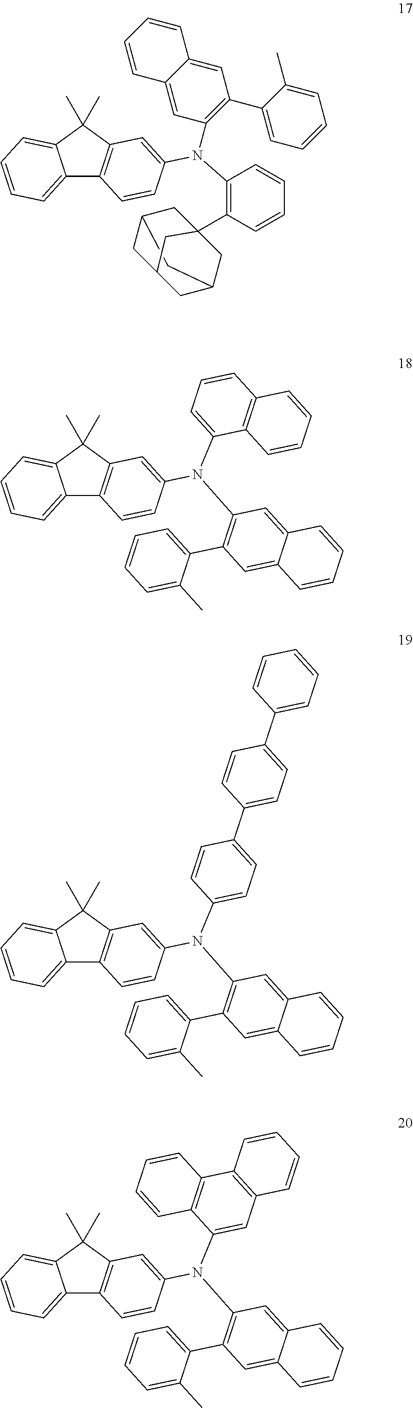 [SMILES: Cc1ccccc1-c1cc2ccccc2cc1N(c1ccc2c(c1)C(C)(C)c1ccccc1-2)c1ccccc1C12CC3CC(CC(C3)C1)C2 ; Cc1ccccc1-c1cc2ccccc2cc1N(c1ccc2c(c1)C(C)(C)c1ccccc1-2)c1cccc2ccccc12 ; Cc1ccccc1-c1cc2ccccc2cc1N(c1ccc(-c2ccc(-c3ccccc3)cc2)cc1)c1ccc2c(c1)C(C)(C)c1ccccc1-2 ; Cc1ccccc1-c1cc2ccccc2cc1N(c1ccc2c(c1)C(C)(C)c1ccccc1-2)c1cc2ccccc2c2ccccc12]

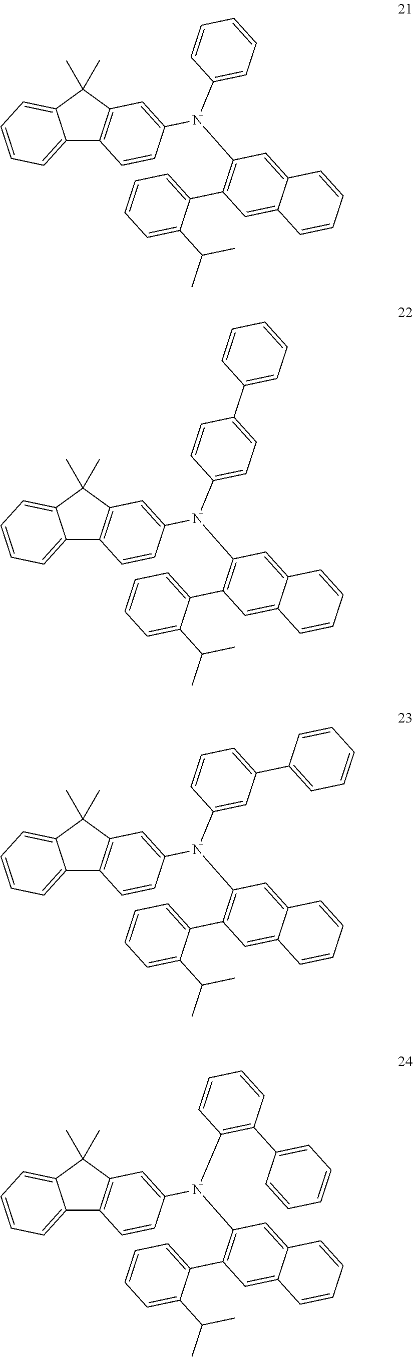 [SMILES: CC(C)c1ccccc1-c1cc2ccccc2cc1N(c1ccccc1)c1ccc2c(c1)C(C)(C)c1ccccc1-2 ; CC(C)c1ccccc1-c1cc2ccccc2cc1N(c1ccc(-c2ccccc2)cc1)c1ccc2c(c1)C(C)(C)c1ccccc1-2 ; CC(C)c1ccccc1-c1cc2ccccc2cc1N(c1cccc(-c2ccccc2)c1)c1ccc2c(c1)C(C)(C)c1ccccc1-2 ; CC(C)c1ccccc1-c1cc2ccccc2cc1N(c1ccc2c(c1)C(C)(C)c1ccccc1-2)c1ccccc1-c1ccccc1]

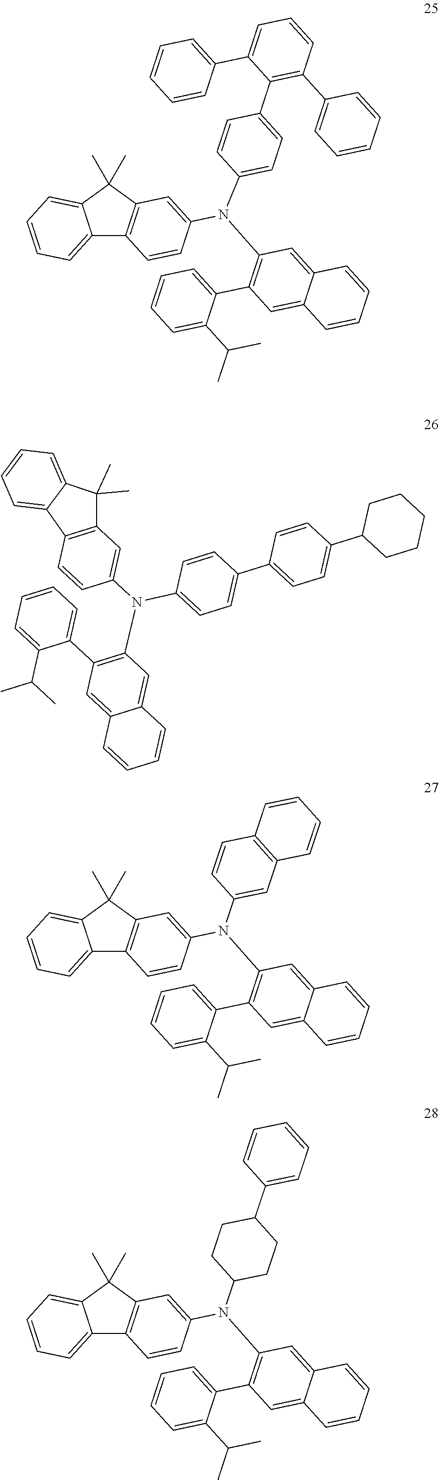 [SMILES: CC(C)c1ccccc1-c1cc2ccccc2cc1N(c1ccc(-c2c(-c3ccccc3)cccc2-c2ccccc2)cc1)c1ccc2c(c1)C(C)(C)c1ccccc1-2 ; CC(C)c1ccccc1-c1cc2ccccc2cc1N(c1ccc(-c2ccc(C3CCCCC3)cc2)cc1)c1ccc2c(c1)C(C)(C)c1ccccc1-2 ; CC(C)c1ccccc1-c1cc2ccccc2cc1N(c1ccc2c(c1)C(C)(C)c1ccccc1-2)c1ccc2ccccc2c1 ; CC(C)c1ccccc1-c1cc2ccccc2cc1N(c1ccc2c(c1)C(C)(C)c1ccccc1-2)C1CCC(c2ccccc2)CC1]

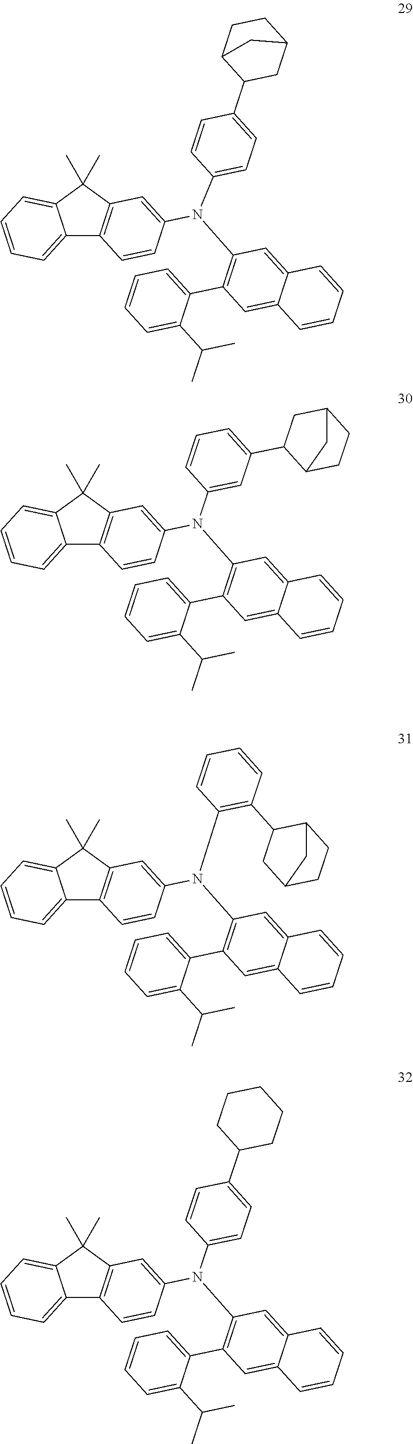 [SMILES: CC(C)c1ccccc1-c1cc2ccccc2cc1N(c1ccc(C2CC3CCC2C3)cc1)c1ccc2c(c1)C(C)(C)c1ccccc1-2 ; CC(C)c1ccccc1-c1cc2ccccc2cc1N(c1cccc(C2CC3CCC2C3)c1)c1ccc2c(c1)C(C)(C)c1ccccc1-2 ; CC(C)c1ccccc1-c1cc2ccccc2cc1N(c1ccc2c(c1)C(C)(C)c1ccccc1-2)c1ccccc1C1CC2CCC1C2 ; CC(C)c1ccccc1-c1cc2ccccc2cc1N(c1ccc(C2CCCCC2)cc1)c1ccc2c(c1)C(C)(C)c1ccccc1-2]

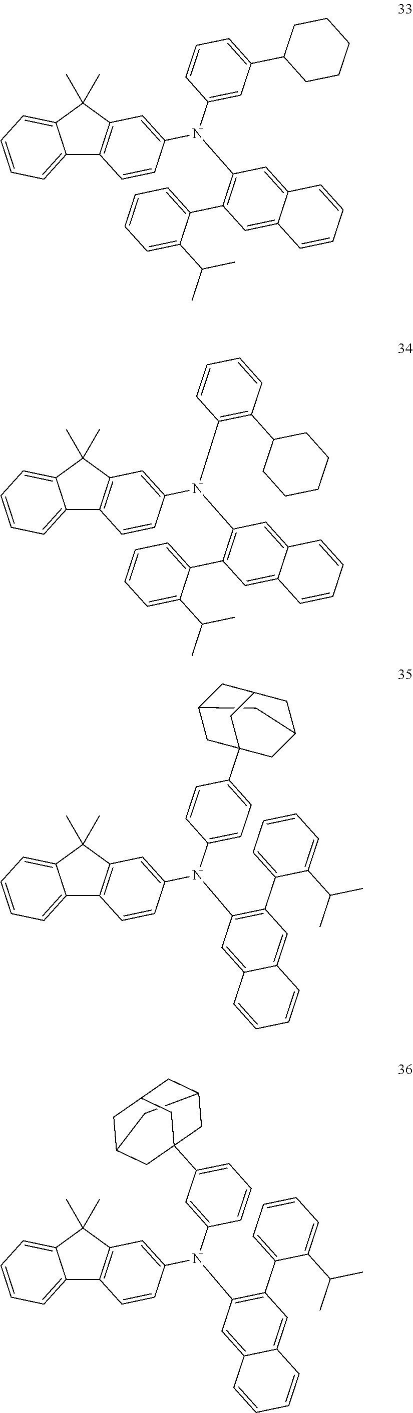

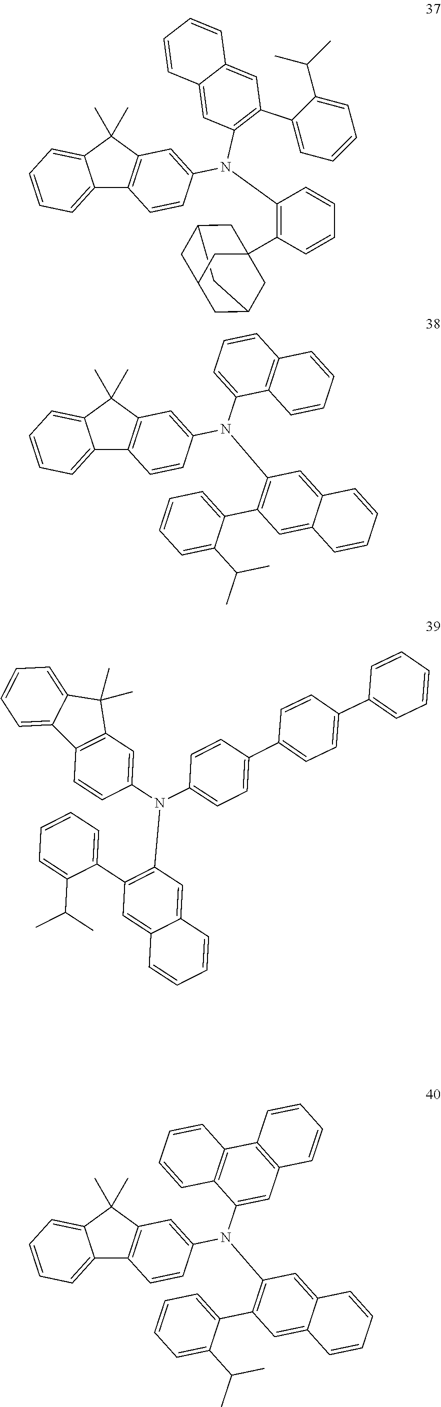

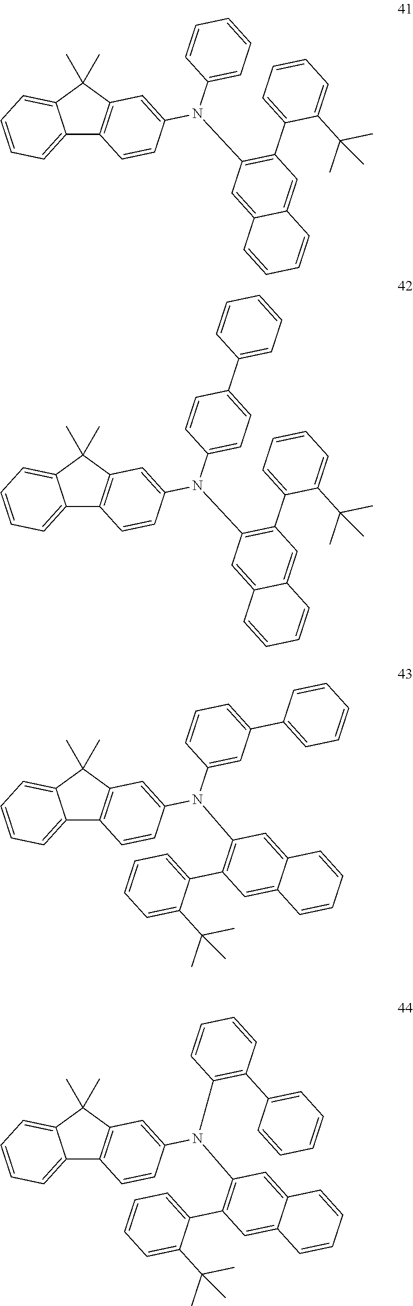

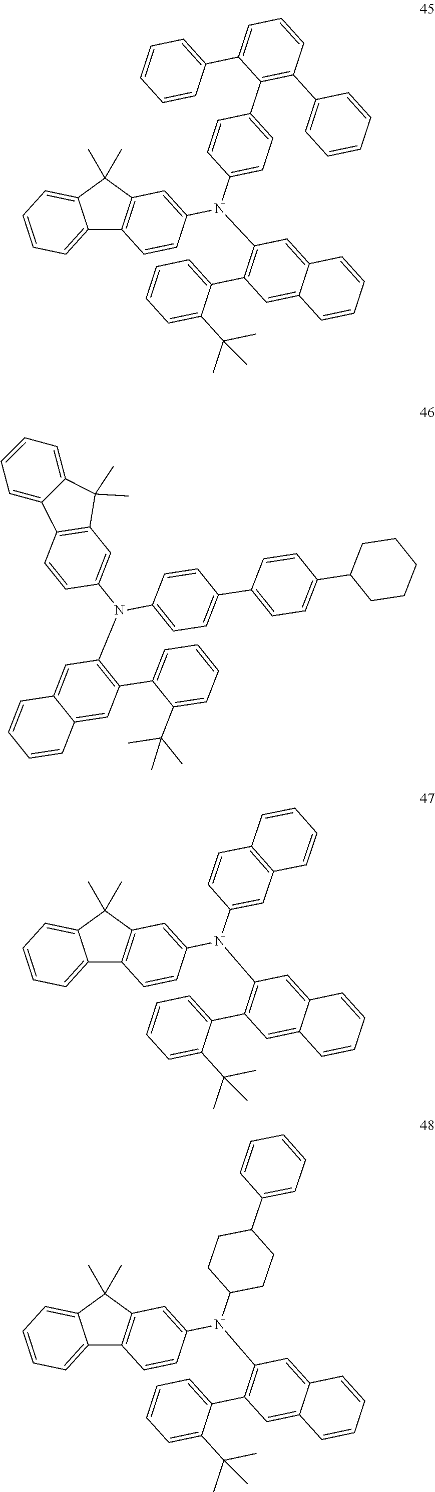

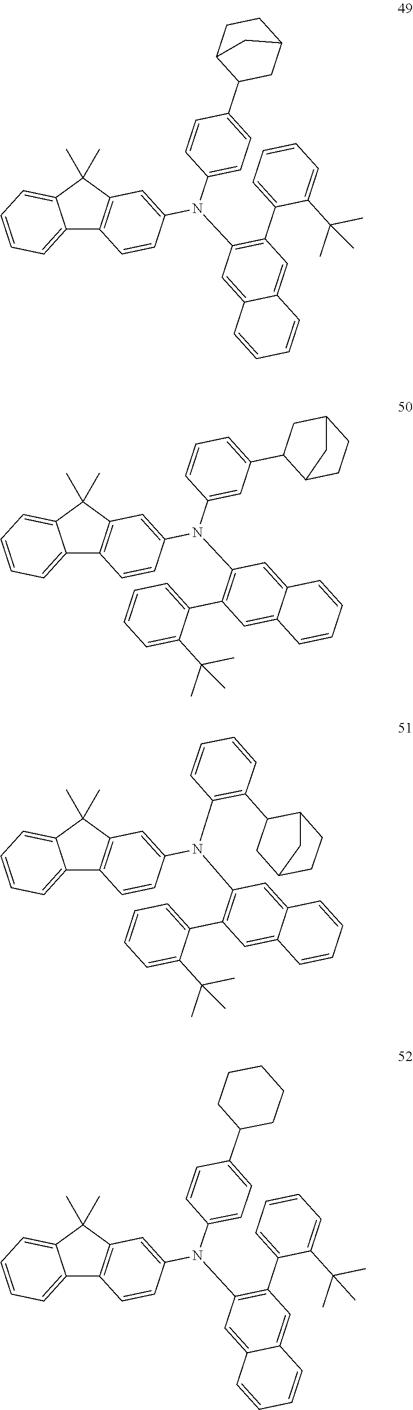 [SMILES: CC(C)(C)c1ccccc1-c1cc2ccccc2cc1N(c1ccc(C2CC3CCC2C3)cc1)c1ccc2c(c1)C(C)(C)c1ccccc1-2 ; CC(C)(C)c1ccccc1-c1cc2ccccc2cc1N(c1cccc(C2CC3CCC2C3)c1)c1ccc2c(c1)C(C)(C)c1ccccc1-2 ; CC(C)(C)c1ccccc1-c1cc2ccccc2cc1N(c1ccc2c(c1)C(C)(C)c1ccccc1-2)c1ccccc1C1CC2CCC1C2 ; CC(C)(C)c1ccccc1-c1cc2ccccc2cc1N(c1ccc(C2CCCCC2)cc1)c1ccc2c(c1)C(C)(C)c1ccccc1-2]

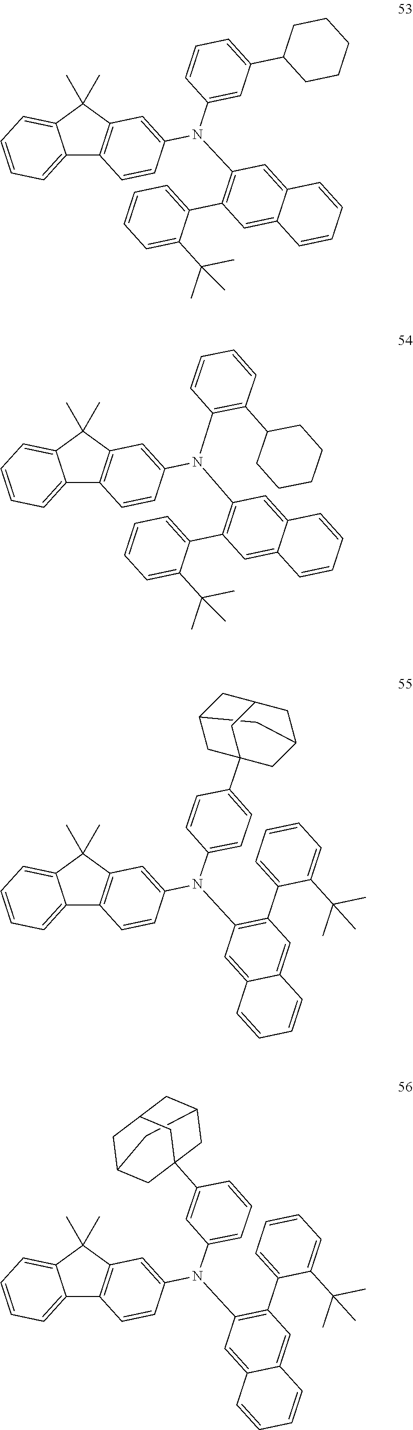 [SMILES: CC(C)(C)c1ccccc1-c1cc2ccccc2cc1N(c1cccc(C2CCCCC2)c1)c1ccc2c(c1)C(C)(C)c1ccccc1-2 ; CC(C)(C)c1ccccc1-c1cc2ccccc2cc1N(c1ccc2c(c1)C(C)(C)c1ccccc1-2)c1ccccc1C1CCCCC1 ; CC(C)(C)c1ccccc1-c1cc2ccccc2cc1N(c1ccc(C23CC4CC(CC(C4)C2)C3)cc1)c1ccc2c(c1)C(C)(C)c1ccccc1-2 ; CC(C)(C)c1ccccc1-c1cc2ccccc2cc1N(c1cccc(C23CC4CC(CC(C4)C2)C3)c1)c1ccc2c(c1)C(C)(C)c1ccccc1-2]

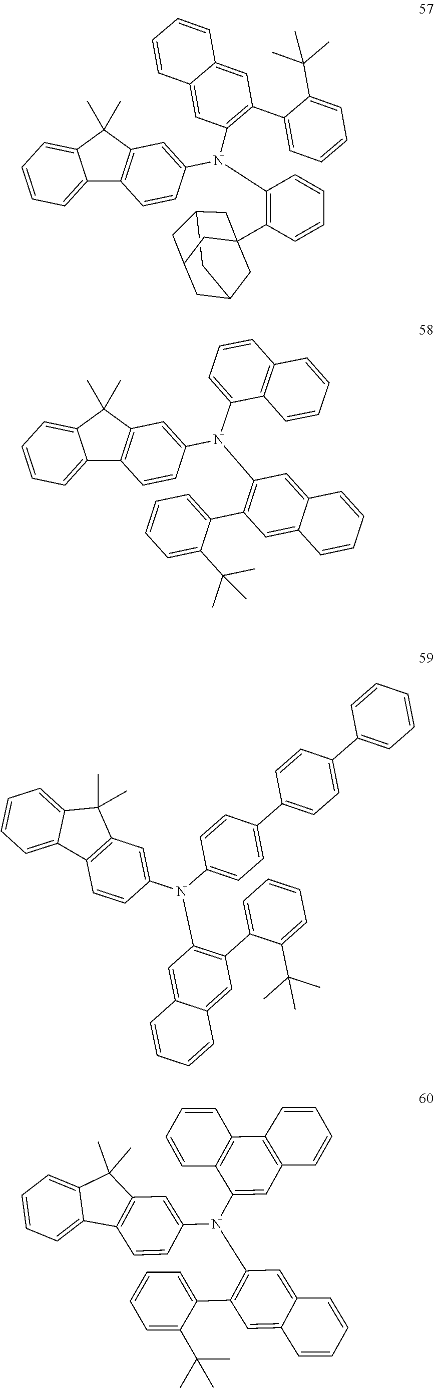

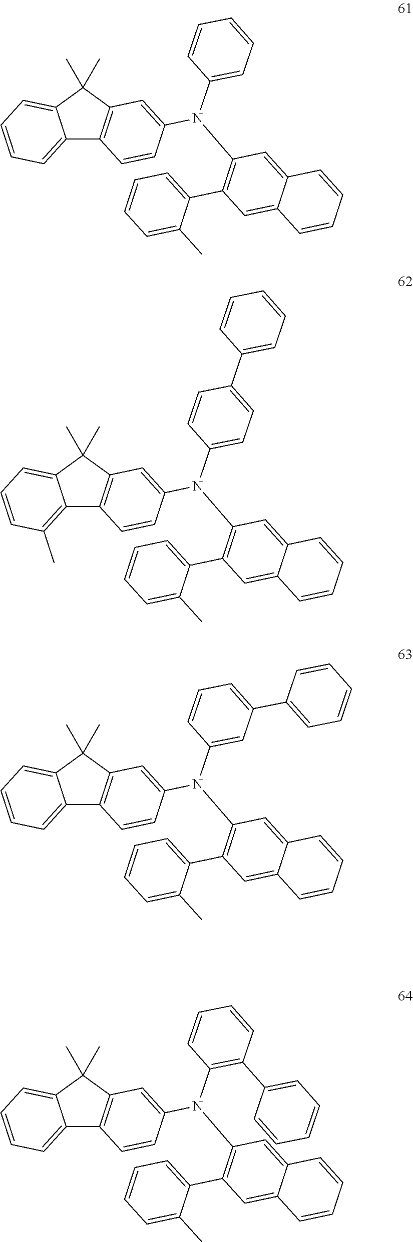 [SMILES: Cc1ccccc1-c1cc2ccccc2cc1N(c1ccccc1)c1ccc2c(c1)C(C)(C)c1ccccc1-2 ; Cc1ccccc1-c1cc2ccccc2cc1N(c1ccc(-c2ccccc2)cc1)c1ccc2c(c1)C(C)(C)c1cccc(C)c1-2 ; Cc1ccccc1-c1cc2ccccc2cc1N(c1cccc(-c2ccccc2)c1)c1ccc2c(c1)C(C)(C)c1ccccc1-2 ; Cc1ccccc1-c1cc2ccccc2cc1N(c1ccc2c(c1)C(C)(C)c1ccccc1-2)c1ccccc1-c1ccccc1]

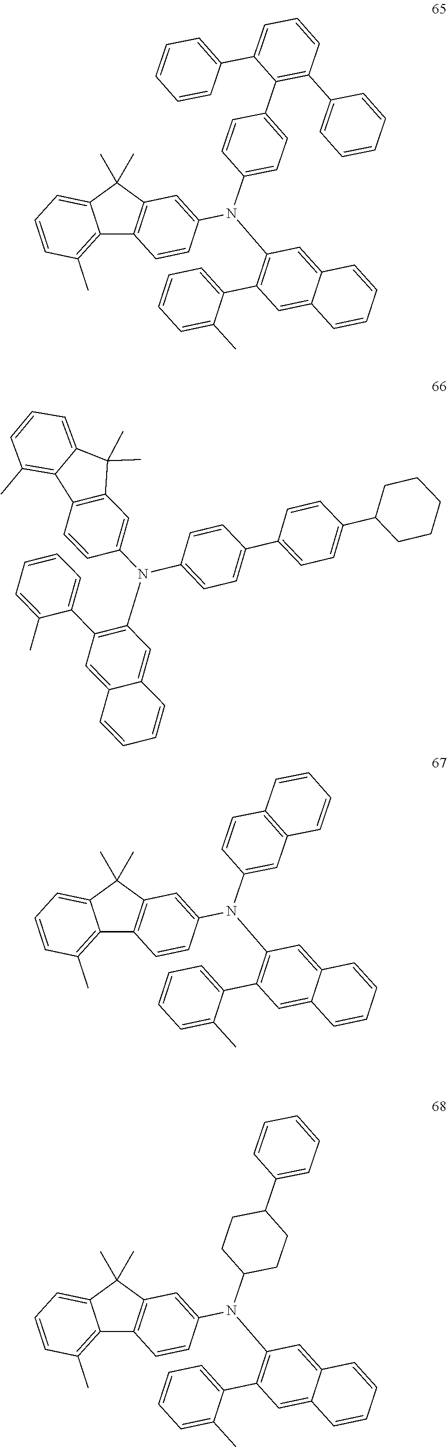 [SMILES: Cc1ccccc1-c1cc2ccccc2cc1N(c1ccc(-c2c(-c3ccccc3)cccc2-c2ccccc2)cc1)c1ccc2c(c1)C(C)(C)c1cccc(C)c1-2 ; Cc1ccccc1-c1cc2ccccc2cc1N(c1ccc(-c2ccc(C3CCCCC3)cc2)cc1)c1ccc2c(c1)C(C)(C)c1cccc(C)c1-2 ; Cc1ccccc1-c1cc2ccccc2cc1N(c1ccc2c(c1)C(C)(C)c1cccc(C)c1-2)c1ccc2ccccc2c1 ; Cc1ccccc1-c1cc2ccccc2cc1N(c1ccc2c(c1)C(C)(C)c1cccc(C)c1-2)C1CCC(c2ccccc2)CC1]

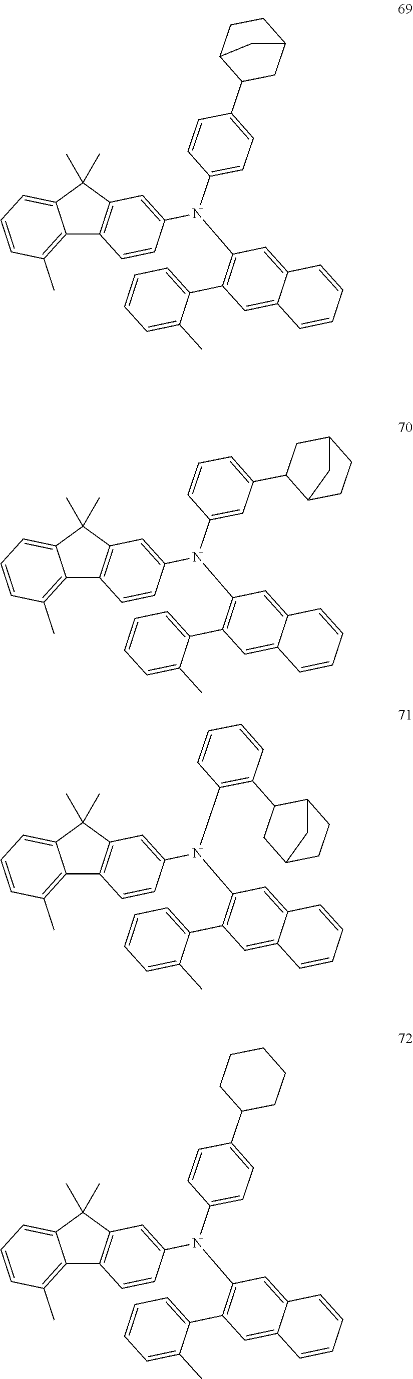

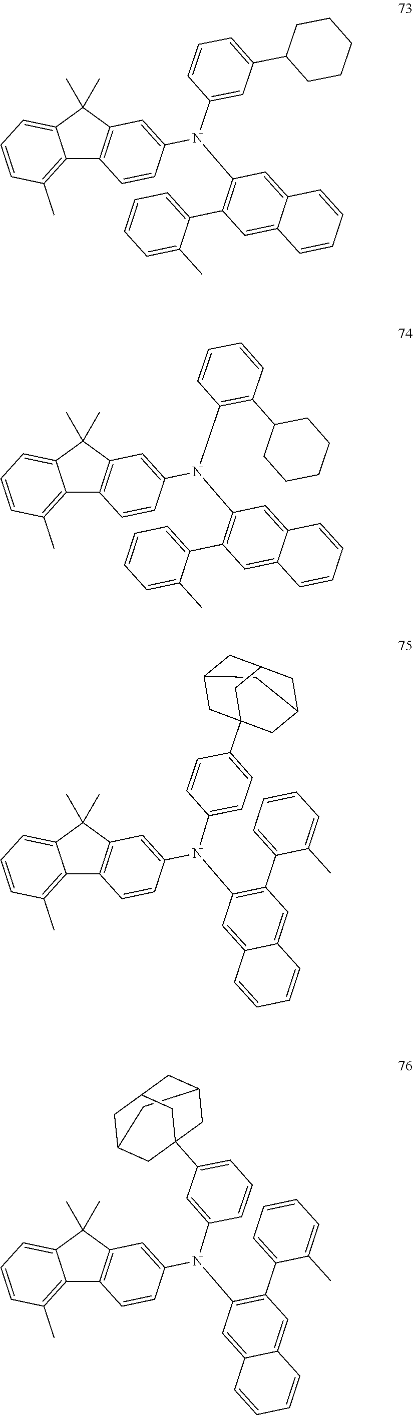 [SMILES: Cc1ccccc1-c1cc2ccccc2cc1N(c1cccc(C2CCCCC2)c1)c1ccc2c(c1)C(C)(C)c1cccc(C)c1-2 ; Cc1ccccc1-c1cc2ccccc2cc1N(c1ccc2c(c1)C(C)(C)c1cccc(C)c1-2)c1ccccc1C1CCCCC1 ; Cc1ccccc1-c1cc2ccccc2cc1N(c1ccc(C23CC4CC(CC(C4)C2)C3)cc1)c1ccc2c(c1)C(C)(C)c1cccc(C)c1-2 ; Cc1ccccc1-c1cc2ccccc2cc1N(c1cccc(C23CC4CC(CC(C4)C2)C3)c1)c1ccc2c(c1)C(C)(C)c1cccc(C)c1-2]

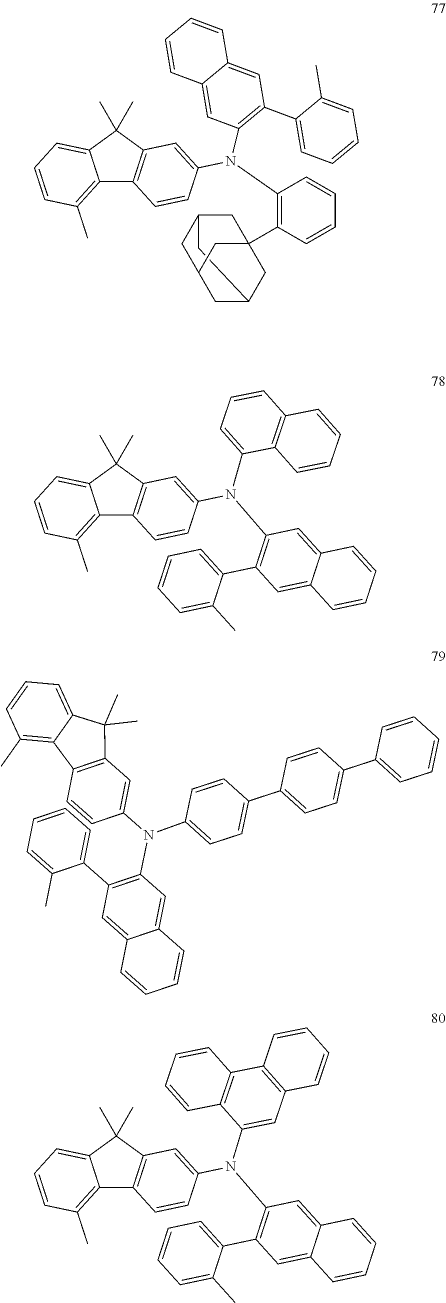 [SMILES: Cc1ccccc1-c1cc2ccccc2cc1N(c1ccc2c(c1)C(C)(C)c1cccc(C)c1-2)c1ccccc1C12CC3CC(CC(C3)C1)C2 ; Cc1ccccc1-c1cc2ccccc2cc1N(c1ccc2c(c1)C(C)(C)c1cccc(C)c1-2)c1cccc2ccccc12 ; Cc1ccccc1-c1cc2ccccc2cc1N(c1ccc(-c2ccc(-c3ccccc3)cc2)cc1)c1ccc2c(c1)C(C)(C)c1cccc(C)c1-2 ; Cc1ccccc1-c1cc2ccccc2cc1N(c1ccc2c(c1)C(C)(C)c1cccc(C)c1-2)c1cc2ccccc2c2ccccc12]

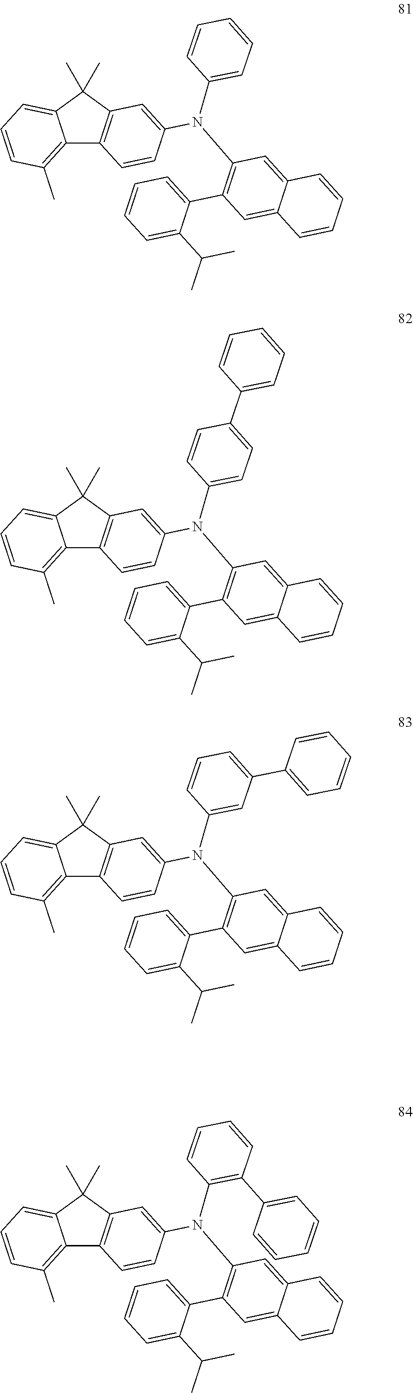 [SMILES: Cc1cccc2c1-c1ccc(N(c3ccccc3)c3cc4ccccc4cc3-c3ccccc3C(C)C)cc1C2(C)C ; Cc1cccc2c1-c1ccc(N(c3ccc(-c4ccccc4)cc3)c3cc4ccccc4cc3-c3ccccc3C(C)C)cc1C2(C)C ; Cc1cccc2c1-c1ccc(N(c3cccc(-c4ccccc4)c3)c3cc4ccccc4cc3-c3ccccc3C(C)C)cc1C2(C)C ; Cc1cccc2c1-c1ccc(N(c3ccccc3-c3ccccc3)c3cc4ccccc4cc3-c3ccccc3C(C)C)cc1C2(C)C]

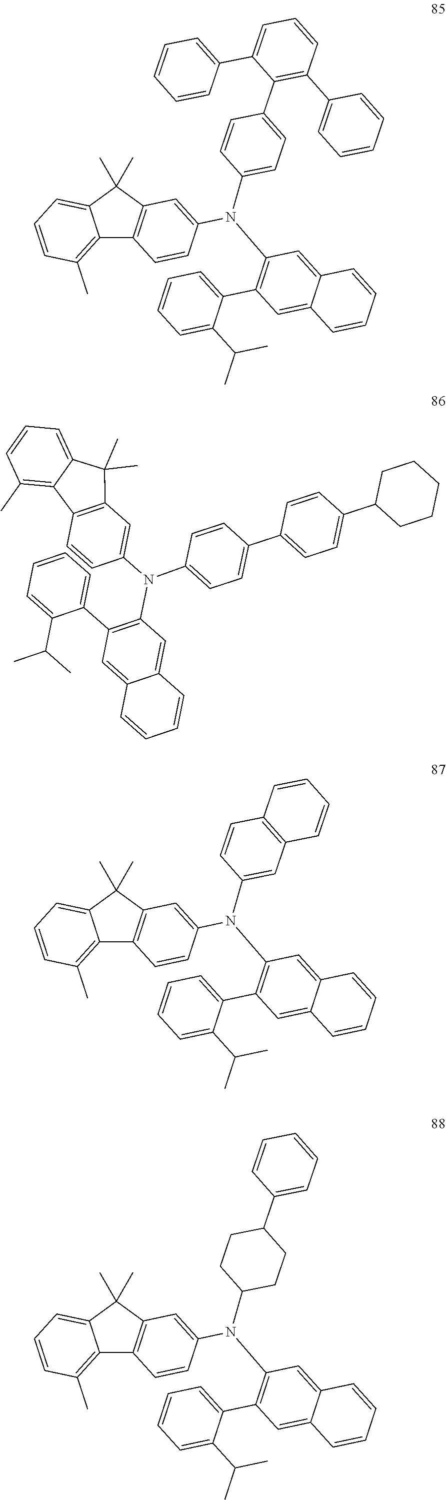 [SMILES: Cc1cccc2c1-c1ccc(N(c3ccc(-c4c(-c5ccccc5)cccc4-c4ccccc4)cc3)c3cc4ccccc4cc3-c3ccccc3C(C)C)cc1C2(C)C ; Cc1cccc2c1-c1ccc(N(c3ccc(-c4ccc(C5CCCCC5)cc4)cc3)c3cc4ccccc4cc3-c3ccccc3C(C)C)cc1C2(C)C ; Cc1cccc2c1-c1ccc(N(c3ccc4ccccc4c3)c3cc4ccccc4cc3-c3ccccc3C(C)C)cc1C2(C)C ; Cc1cccc2c1-c1ccc(N(c3cc4ccccc4cc3-c3ccccc3C(C)C)C3CCC(c4ccccc4)CC3)cc1C2(C)C]

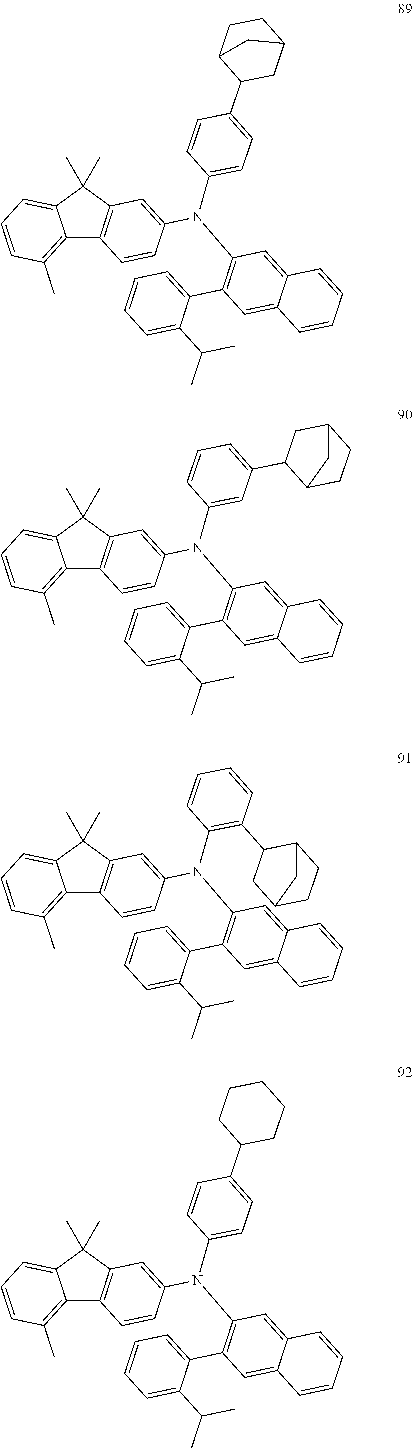 [SMILES: Cc1cccc2c1-c1ccc(N(c3ccc(C4CC5CCC4C5)cc3)c3cc4ccccc4cc3-c3ccccc3C(C)C)cc1C2(C)C ; Cc1cccc2c1-c1ccc(N(c3cccc(C4CC5CCC4C5)c3)c3cc4ccccc4cc3-c3ccccc3C(C)C)cc1C2(C)C ; Cc1cccc2c1-c1ccc(N(c3cc4ccccc4cc3-c3ccccc3C(C)C)c3ccccc3C3CC4CCC3C4)cc1C2(C)C ; Cc1cccc2c1-c1ccc(N(c3ccc(C4CCCCC4)cc3)c3cc4ccccc4cc3-c3ccccc3C(C)C)cc1C2(C)C]

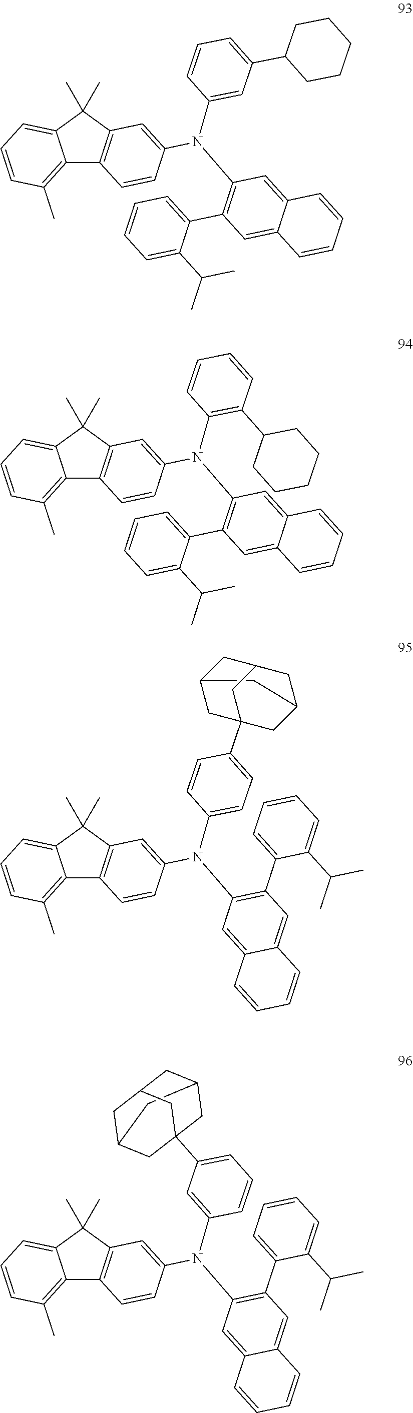

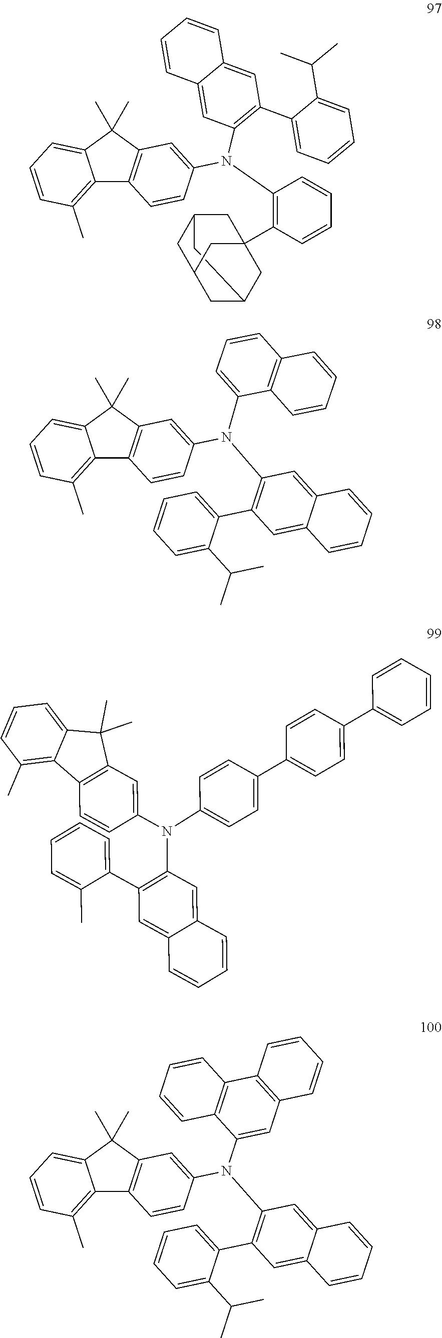

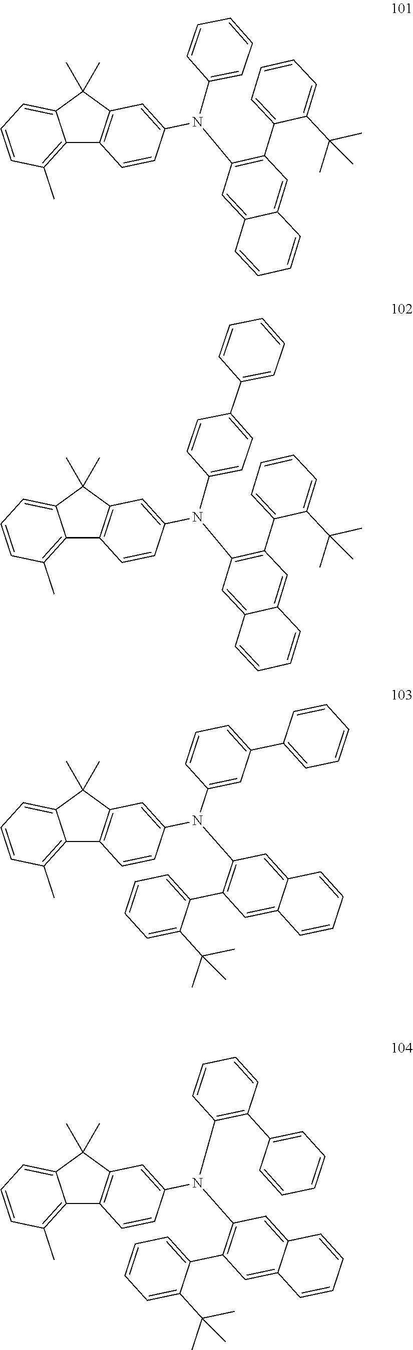

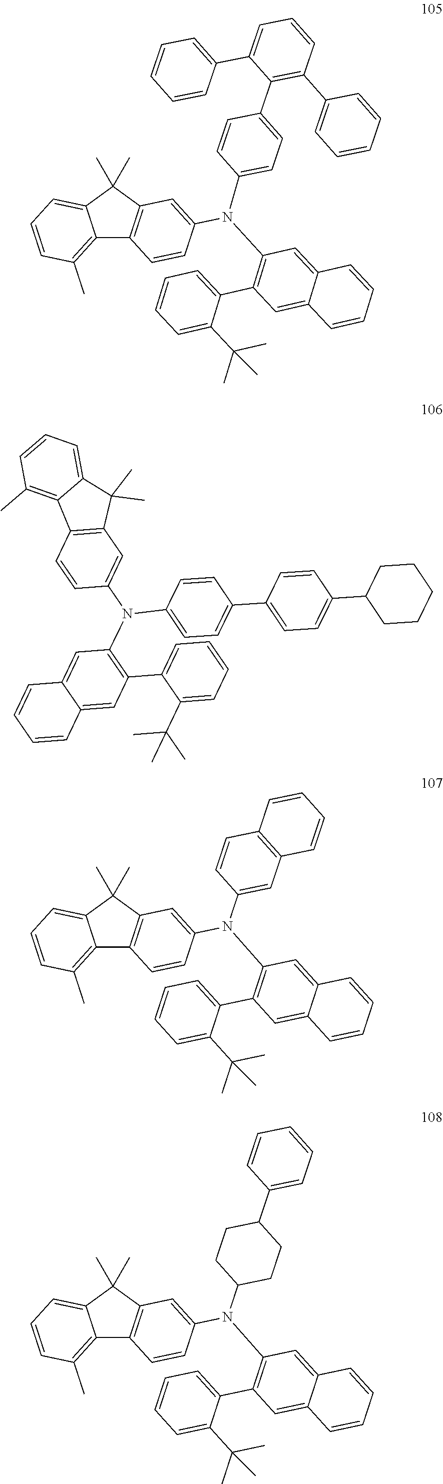 [SMILES: Cc1cccc2c1-c1ccc(N(c3ccc(-c4c(-c5ccccc5)cccc4-c4ccccc4)cc3)c3cc4ccccc4cc3-c3ccccc3C(C)(C)C)cc1C2(C)C ; Cc1cccc2c1-c1ccc(N(c3ccc(-c4ccc(C5CCCCC5)cc4)cc3)c3cc4ccccc4cc3-c3ccccc3C(C)(C)C)cc1C2(C)C ; Cc1cccc2c1-c1ccc(N(c3ccc4ccccc4c3)c3cc4ccccc4cc3-c3ccccc3C(C)(C)C)cc1C2(C)C ; Cc1cccc2c1-c1ccc(N(c3cc4ccccc4cc3-c3ccccc3C(C)(C)C)C3CCC(c4ccccc4)CC3)cc1C2(C)C]

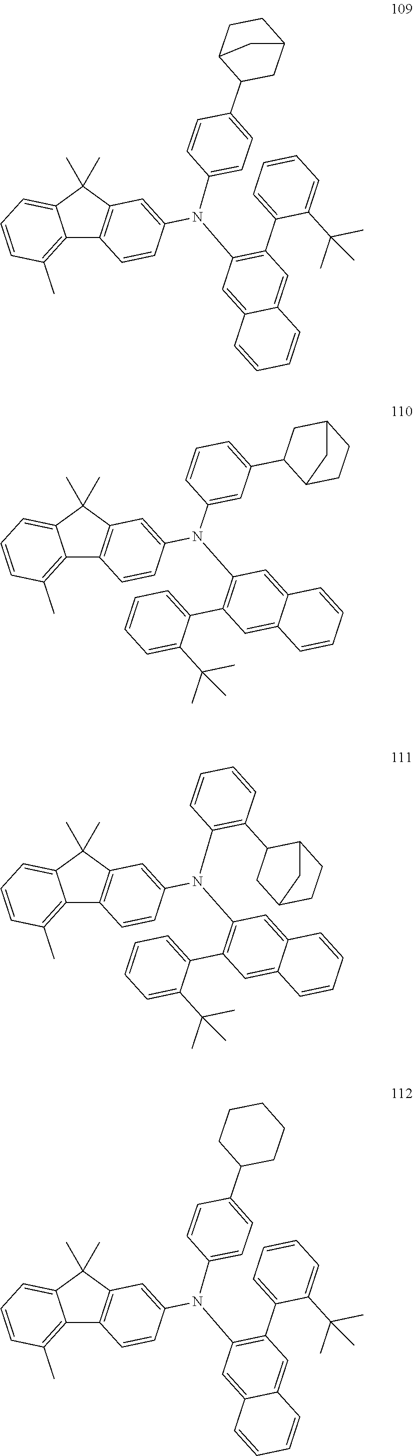 [SMILES: Cc1cccc2c1-c1ccc(N(c3ccc(C4CC5CCC4C5)cc3)c3cc4ccccc4cc3-c3ccccc3C(C)(C)C)cc1C2(C)C ; Cc1cccc2c1-c1ccc(N(c3cccc(C4CC5CCC4C5)c3)c3cc4ccccc4cc3-c3ccccc3C(C)(C)C)cc1C2(C)C ; Cc1cccc2c1-c1ccc(N(c3cc4ccccc4cc3-c3ccccc3C(C)(C)C)c3ccccc3C3CC4CCC3C4)cc1C2(C)C ; Cc1cccc2c1-c1ccc(N(c3ccc(C4CCCCC4)cc3)c3cc4ccccc4cc3-c3ccccc3C(C)(C)C)cc1C2(C)C]

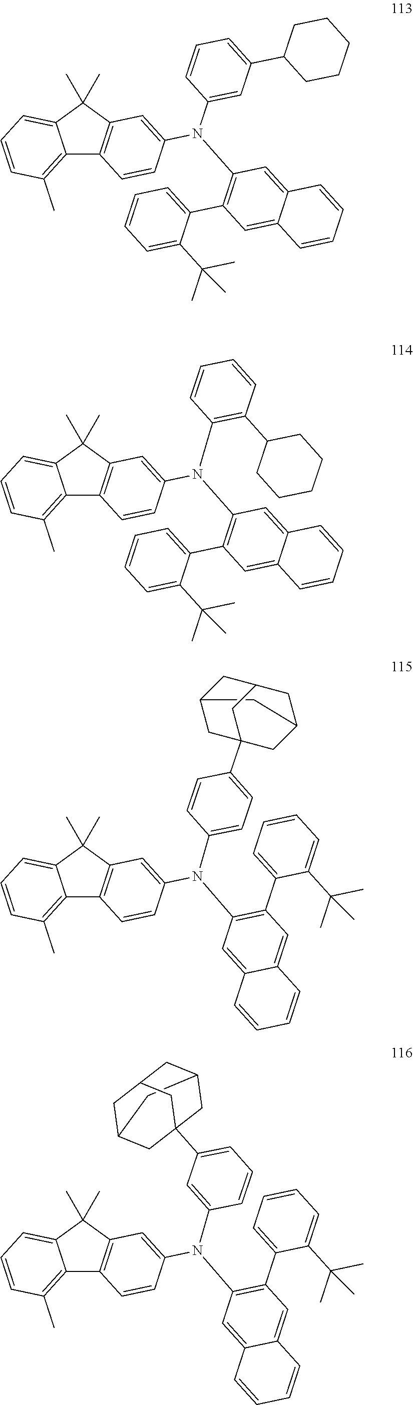

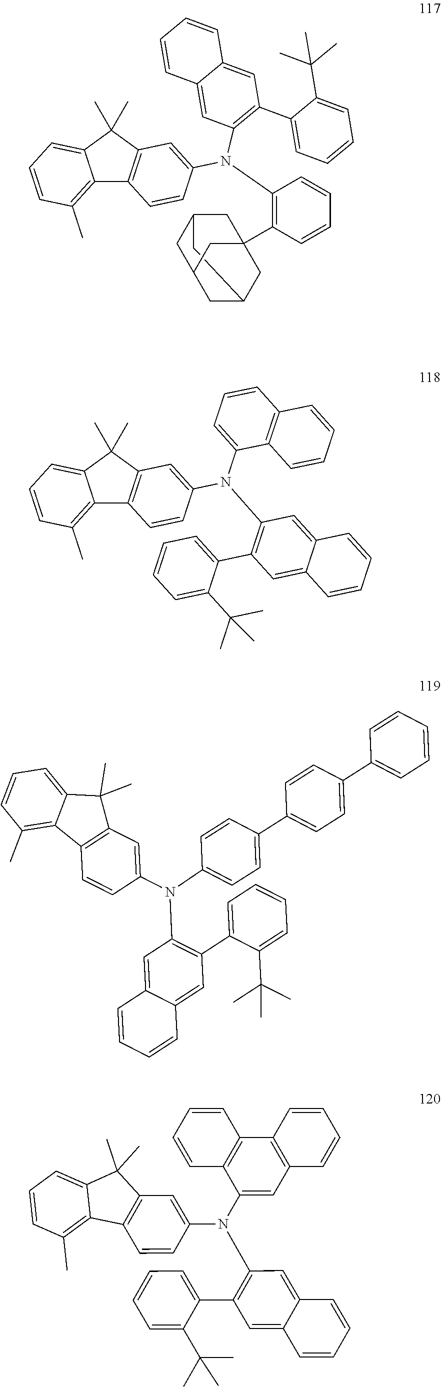 [SMILES: Cc1cccc2c1-c1ccc(N(c3cc4ccccc4cc3-c3ccccc3C(C)(C)C)c3ccccc3C34CC5CC(CC(C5)C3)C4)cc1C2(C)C ; Cc1cccc2c1-c1ccc(N(c3cc4ccccc4cc3-c3ccccc3C(C)(C)C)c3cccc4ccccc34)cc1C2(C)C ; Cc1cccc2c1-c1ccc(N(c3ccc(-c4ccc(-c5ccccc5)cc4)cc3)c3cc4ccccc4cc3-c3ccccc3C(C)(C)C)cc1C2(C)C ; Cc1cccc2c1-c1ccc(N(c3cc4ccccc4cc3-c3ccccc3C(C)(C)C)c3cc4ccccc4c4ccccc34)cc1C2(C)C]

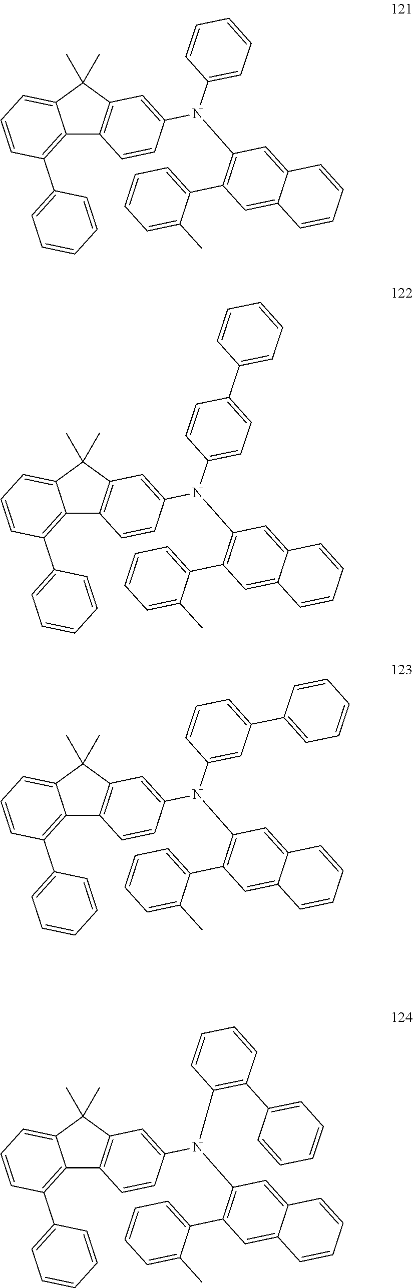 [SMILES: Cc1ccccc1-c1cc2ccccc2cc1N(c1ccccc1)c1ccc2c(c1)C(C)(C)c1cccc(-c3ccccc3)c1-2 ; Cc1ccccc1-c1cc2ccccc2cc1N(c1ccc(-c2ccccc2)cc1)c1ccc2c(c1)C(C)(C)c1cccc(-c3ccccc3)c1-2 ; Cc1ccccc1-c1cc2ccccc2cc1N(c1cccc(-c2ccccc2)c1)c1ccc2c(c1)C(C)(C)c1cccc(-c3ccccc3)c1-2 ; Cc1ccccc1-c1cc2ccccc2cc1N(c1ccc2c(c1)C(C)(C)c1cccc(-c3ccccc3)c1-2)c1ccccc1-c1ccccc1]

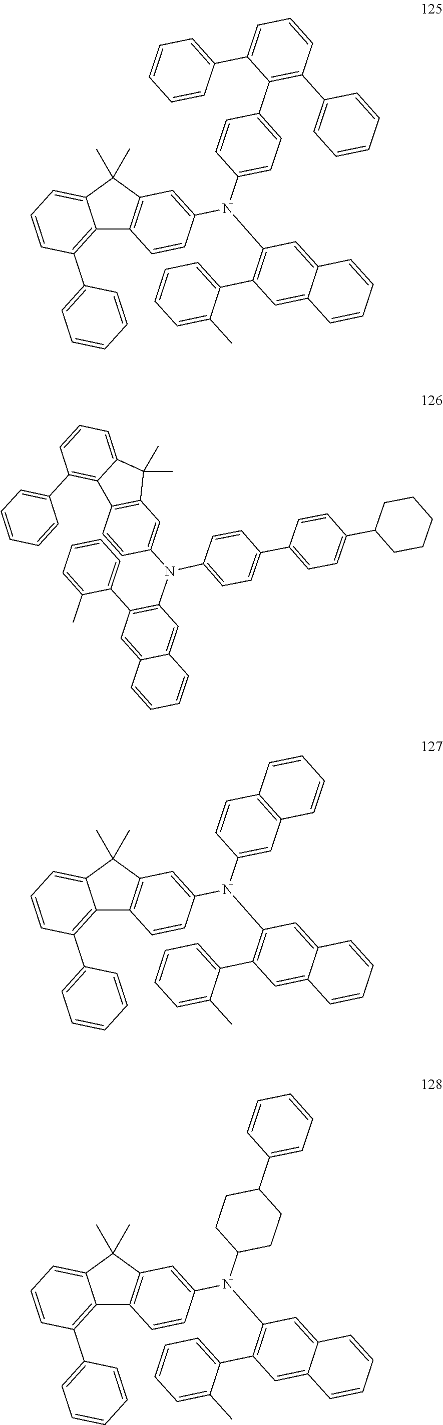 [SMILES: Cc1ccccc1-c1cc2ccccc2cc1N(c1ccc(-c2c(-c3ccccc3)cccc2-c2ccccc2)cc1)c1ccc2c(c1)C(C)(C)c1cccc(-c3ccccc3)c1-2 ; Cc1ccccc1-c1cc2ccccc2cc1N(c1ccc(-c2ccc(C3CCCCC3)cc2)cc1)c1ccc2c(c1)C(C)(C)c1cccc(-c3ccccc3)c1-2 ; Cc1ccccc1-c1cc2ccccc2cc1N(c1ccc2c(c1)C(C)(C)c1cccc(-c3ccccc3)c1-2)c1ccc2ccccc2c1 ; Cc1ccccc1-c1cc2ccccc2cc1N(c1ccc2c(c1)C(C)(C)c1cccc(-c3ccccc3)c1-2)C1CCC(c2ccccc2)CC1]

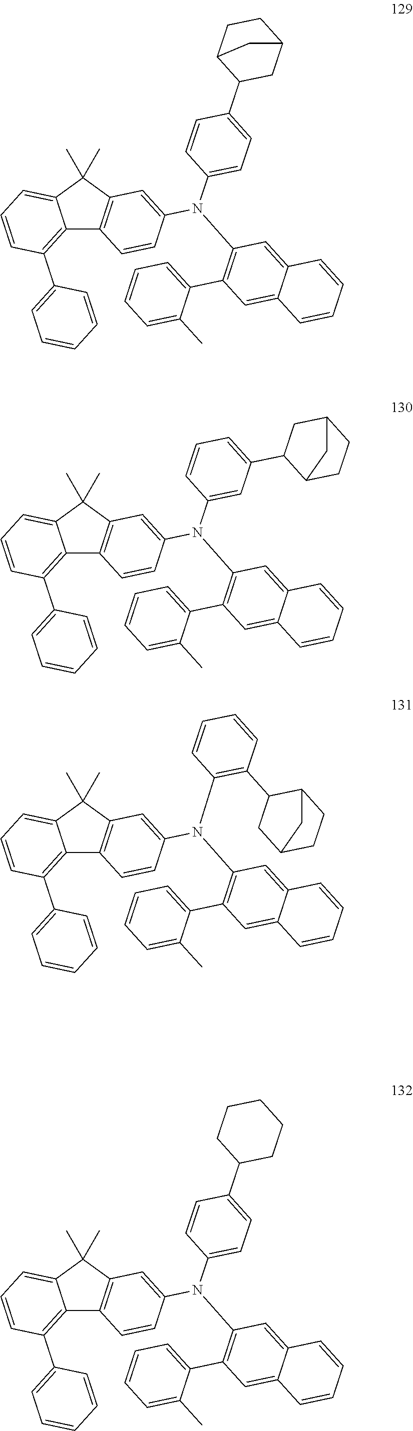 [SMILES: Cc1ccccc1-c1cc2ccccc2cc1N(c1ccc(C2CC3CCC2C3)cc1)c1ccc2c(c1)C(C)(C)c1cccc(-c3ccccc3)c1-2 ; Cc1ccccc1-c1cc2ccccc2cc1N(c1cccc(C2CC3CCC2C3)c1)c1ccc2c(c1)C(C)(C)c1cccc(-c3ccccc3)c1-2 ; Cc1ccccc1-c1cc2ccccc2cc1N(c1ccc2c(c1)C(C)(C)c1cccc(-c3ccccc3)c1-2)c1ccccc1C1CC2CCC1C2 ; Cc1ccccc1-c1cc2ccccc2cc1N(c1ccc(C2CCCCC2)cc1)c1ccc2c(c1)C(C)(C)c1cccc(-c3ccccc3)c1-2]

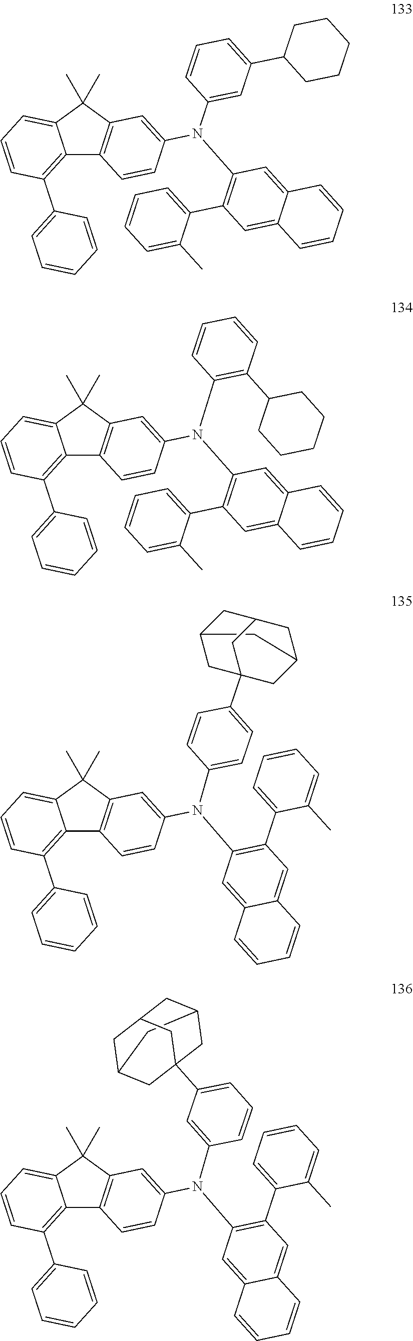

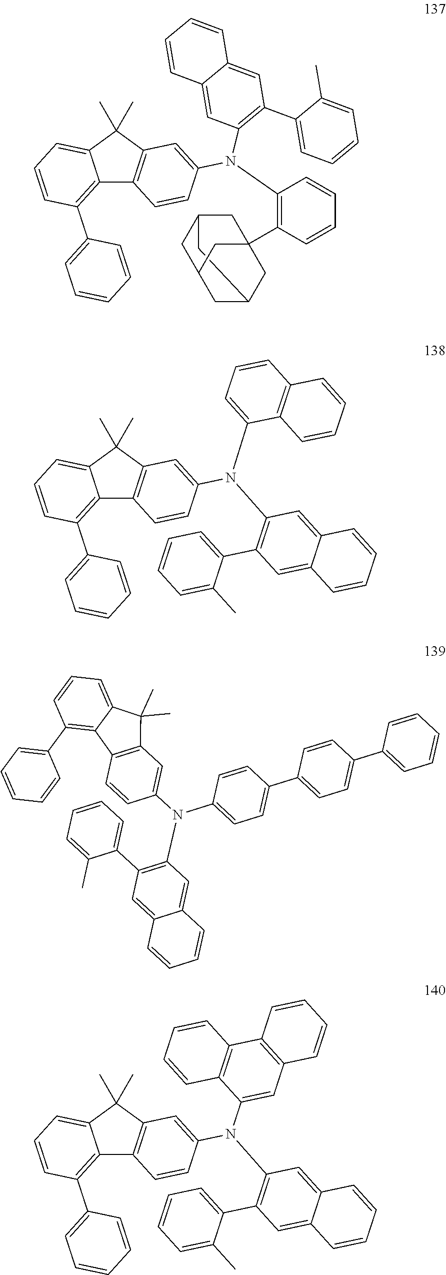 [SMILES: Cc1ccccc1-c1cc2ccccc2cc1N(c1ccc2c(c1)C(C)(C)c1cccc(-c3ccccc3)c1-2)c1ccccc1C12CC3CC(CC(C3)C1)C2 ; Cc1ccccc1-c1cc2ccccc2cc1N(c1ccc2c(c1)C(C)(C)c1cccc(-c3ccccc3)c1-2)c1cccc2ccccc12 ; Cc1ccccc1-c1cc2ccccc2cc1N(c1ccc(-c2ccc(-c3ccccc3)cc2)cc1)c1ccc2c(c1)C(C)(C)c1cccc(-c3ccccc3)c1-2 ; Cc1ccccc1-c1cc2ccccc2cc1N(c1ccc2c(c1)C(C)(C)c1cccc(-c3ccccc3)c1-2)c1cc2ccccc2c2ccccc12]

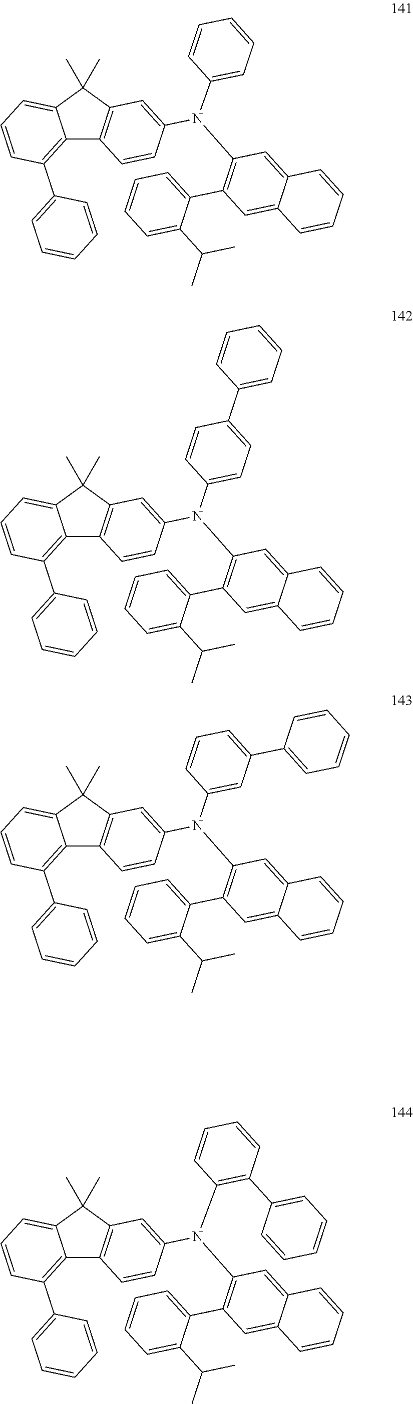 [SMILES: CC(C)c1ccccc1-c1cc2ccccc2cc1N(c1ccccc1)c1ccc2c(c1)C(C)(C)c1cccc(-c3ccccc3)c1-2 ; CC(C)c1ccccc1-c1cc2ccccc2cc1N(c1ccc(-c2ccccc2)cc1)c1ccc2c(c1)C(C)(C)c1cccc(-c3ccccc3)c1-2 ; CC(C)c1ccccc1-c1cc2ccccc2cc1N(c1cccc(-c2ccccc2)c1)c1ccc2c(c1)C(C)(C)c1cccc(-c3ccccc3)c1-2 ; CC(C)c1ccccc1-c1cc2ccccc2cc1N(c1ccc2c(c1)C(C)(C)c1cccc(-c3ccccc3)c1-2)c1ccccc1-c1ccccc1]

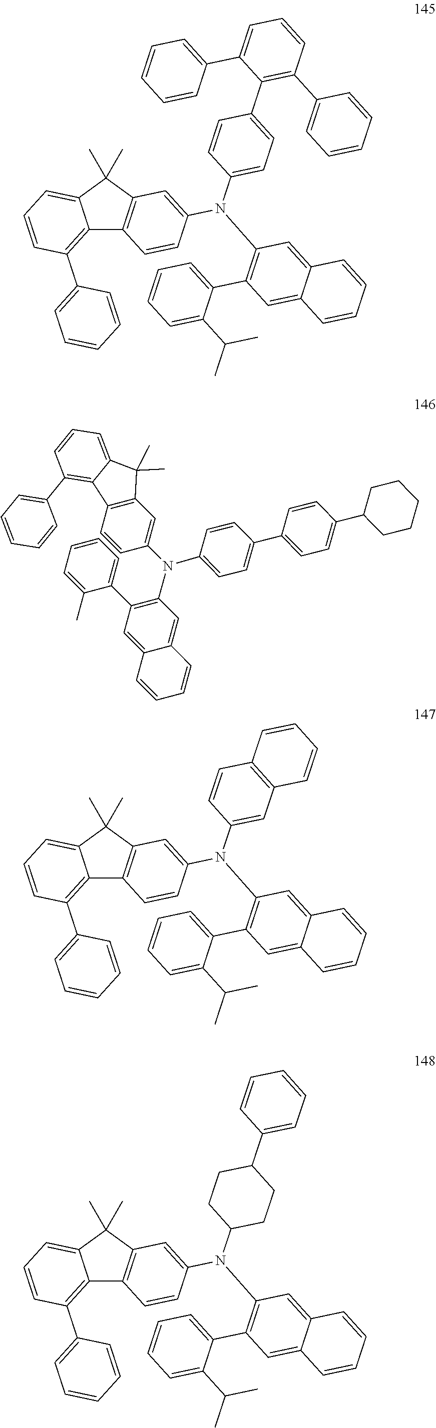 [SMILES: CC(C)c1ccccc1-c1cc2ccccc2cc1N(c1ccc(-c2c(-c3ccccc3)cccc2-c2ccccc2)cc1)c1ccc2c(c1)C(C)(C)c1cccc(-c3ccccc3)c1-2 ; Cc1ccccc1-c1cc2ccccc2cc1N(c1ccc(-c2ccc(C3CCCCC3)cc2)cc1)c1ccc2c(c1)C(C)(C)c1cccc(-c3ccccc3)c1-2 ; CC(C)c1ccccc1-c1cc2ccccc2cc1N(c1ccc2c(c1)C(C)(C)c1cccc(-c3ccccc3)c1-2)c1ccc2ccccc2c1 ; CC(C)c1ccccc1-c1cc2ccccc2cc1N(c1ccc2c(c1)C(C)(C)c1cccc(-c3ccccc3)c1-2)C1CCC(c2ccccc2)CC1]

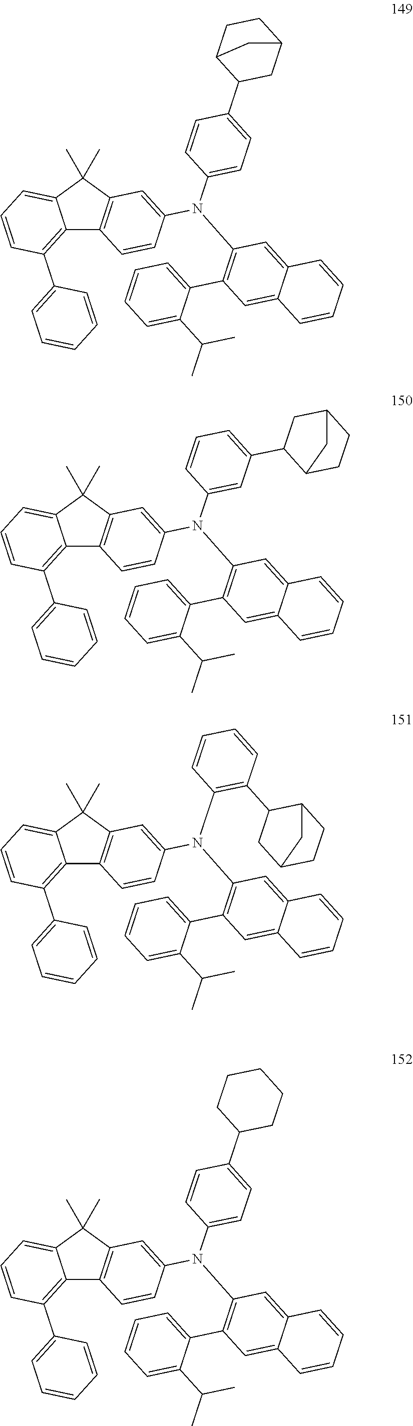

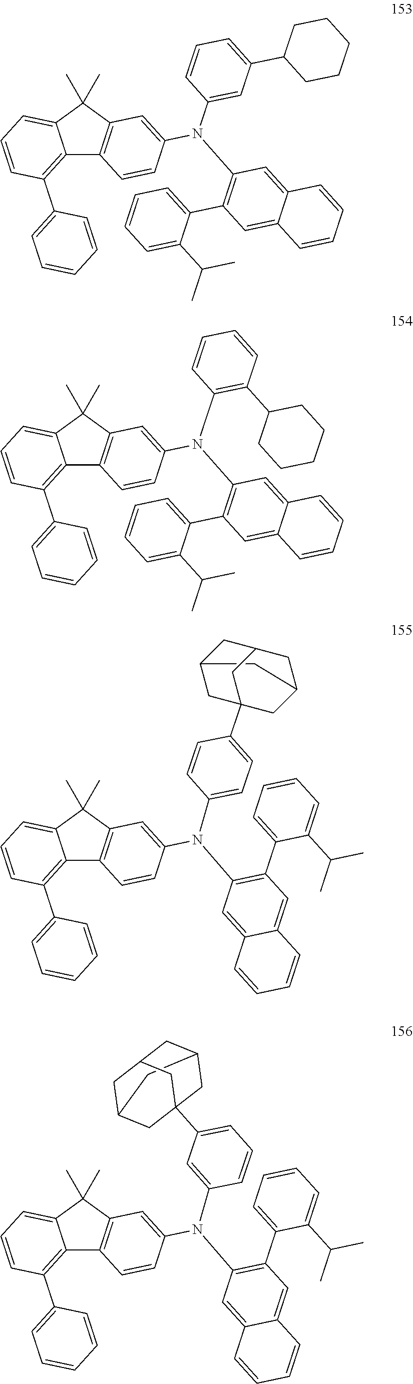

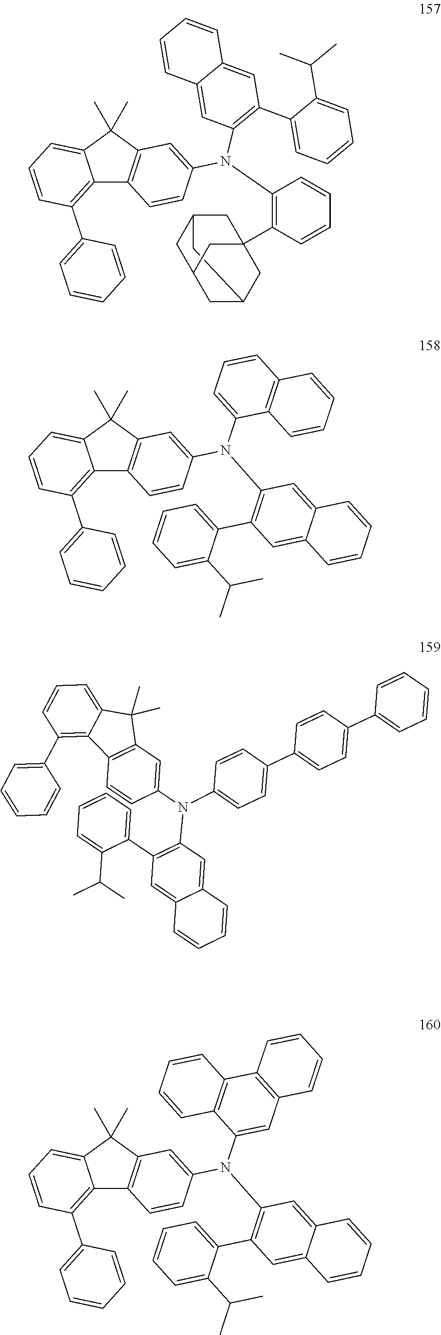 [SMILES: CC(C)c1ccccc1-c1cc2ccccc2cc1N(c1ccc2c(c1)C(C)(C)c1cccc(-c3ccccc3)c1-2)c1ccccc1C12CC3CC(CC(C3)C1)C2 ; CC(C)c1ccccc1-c1cc2ccccc2cc1N(c1ccc2c(c1)C(C)(C)c1cccc(-c3ccccc3)c1-2)c1cccc2ccccc12 ; CC(C)c1ccccc1-c1cc2ccccc2cc1N(c1ccc(-c2ccc(-c3ccccc3)cc2)cc1)c1ccc2c(c1)C(C)(C)c1cccc(-c3ccccc3)c1-2 ; CC(C)c1ccccc1-c1cc2ccccc2cc1N(c1ccc2c(c1)C(C)(C)c1cccc(-c3ccccc3)c1-2)c1cc2ccccc2c2ccccc12]

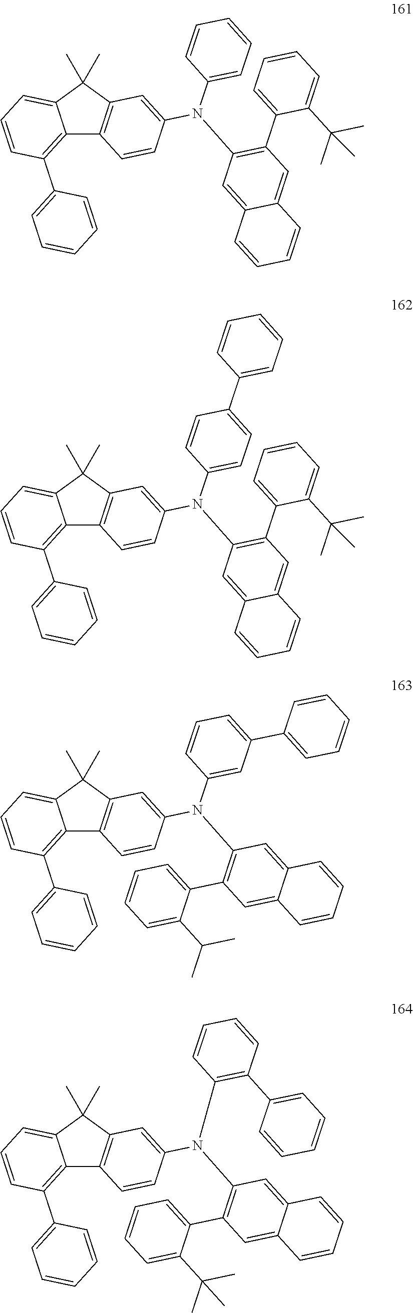 [SMILES: CC(C)(C)c1ccccc1-c1cc2ccccc2cc1N(c1ccccc1)c1ccc2c(c1)C(C)(C)c1cccc(-c3ccccc3)c1-2 ; CC(C)(C)c1ccccc1-c1cc2ccccc2cc1N(c1ccc(-c2ccccc2)cc1)c1ccc2c(c1)C(C)(C)c1cccc(-c3ccccc3)c1-2 ; CC(C)c1ccccc1-c1cc2ccccc2cc1N(c1cccc(-c2ccccc2)c1)c1ccc2c(c1)C(C)(C)c1cccc(-c3ccccc3)c1-2 ; CC(C)(C)c1ccccc1-c1cc2ccccc2cc1N(c1ccc2c(c1)C(C)(C)c1cccc(-c3ccccc3)c1-2)c1ccccc1-c1ccccc1]

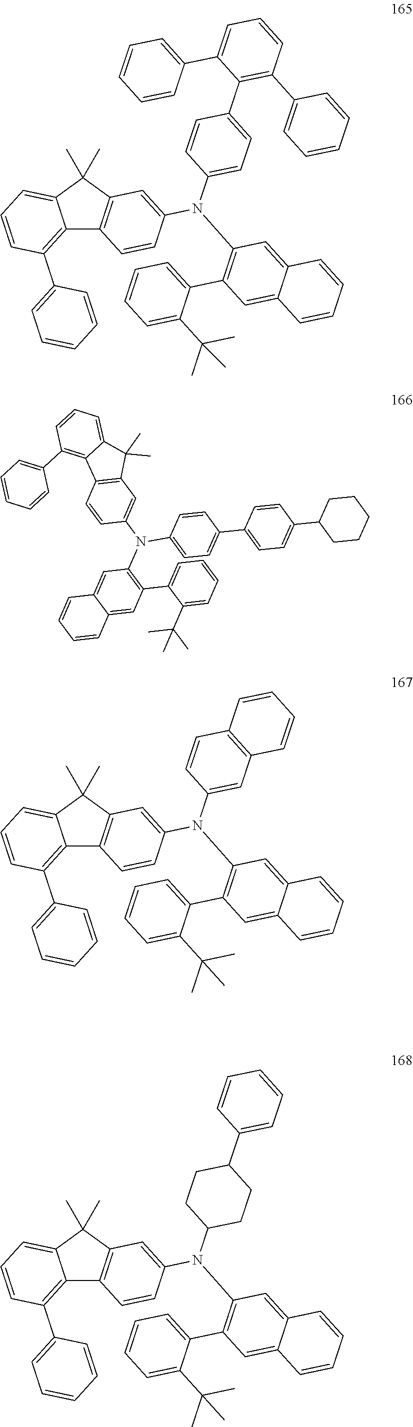 [SMILES: CC(C)(C)c1ccccc1-c1cc2ccccc2cc1N(c1ccc(-c2c(-c3ccccc3)cccc2-c2ccccc2)cc1)c1ccc2c(c1)C(C)(C)c1cccc(-c3ccccc3)c1-2 ; CC(C)(C)c1ccccc1-c1cc2ccccc2cc1N(c1ccc(-c2ccc(C3CCCCC3)cc2)cc1)c1ccc2c(c1)C(C)(C)c1cccc(-c3ccccc3)c1-2 ; CC(C)(C)c1ccccc1-c1cc2ccccc2cc1N(c1ccc2c(c1)C(C)(C)c1cccc(-c3ccccc3)c1-2)c1ccc2ccccc2c1 ; CC(C)(C)c1ccccc1-c1cc2ccccc2cc1N(c1ccc2c(c1)C(C)(C)c1cccc(-c3ccccc3)c1-2)C1CCC(c2ccccc2)CC1]

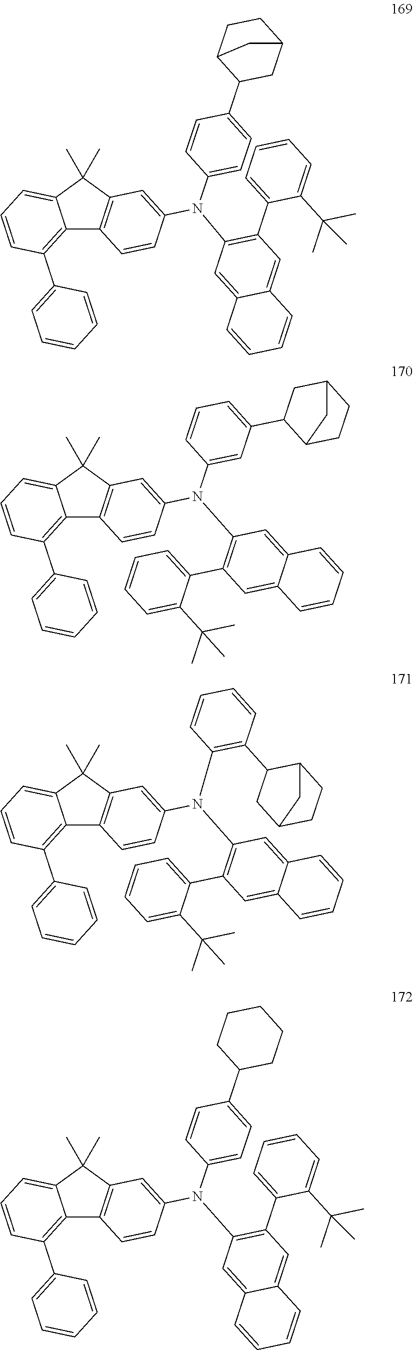

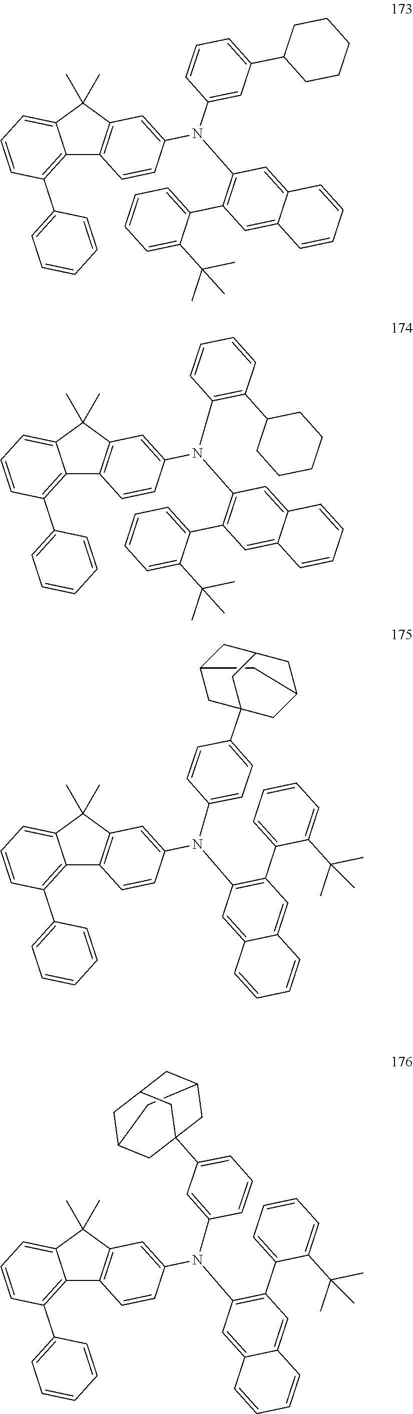

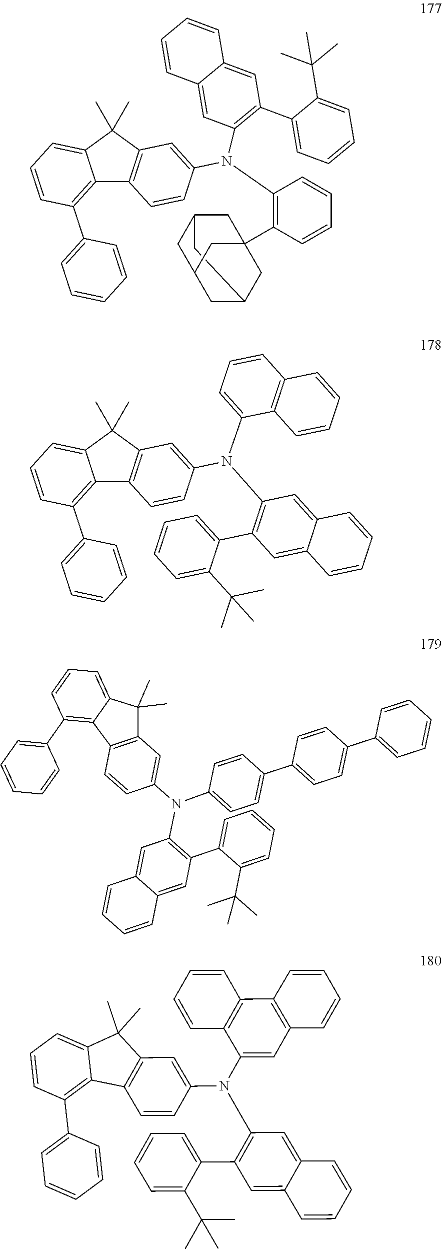

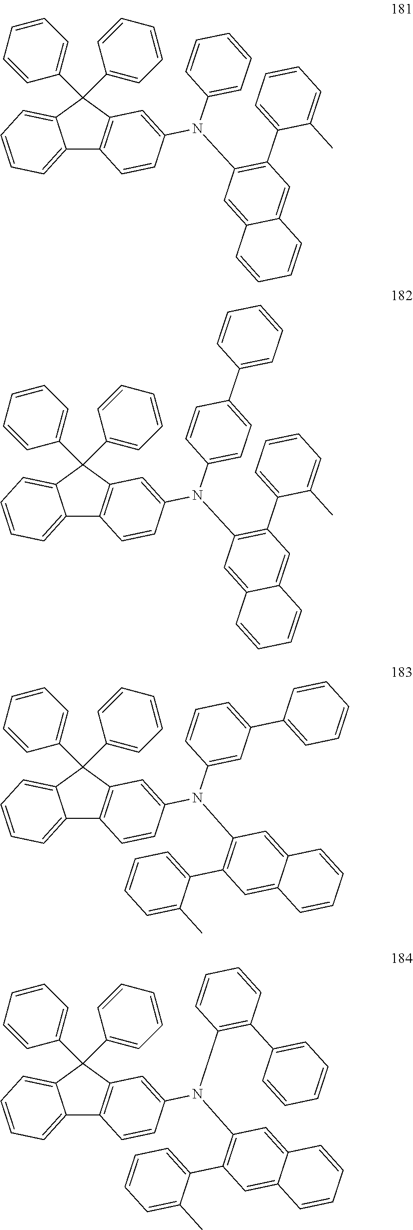

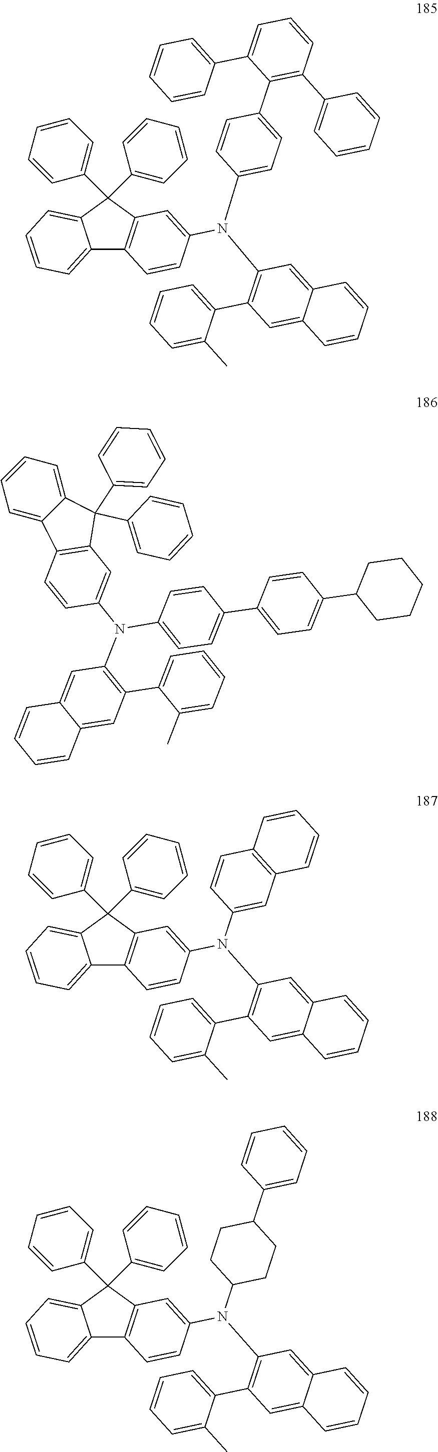 [SMILES: Cc1ccccc1-c1cc2ccccc2cc1N(c1ccc(-c2c(-c3ccccc3)cccc2-c2ccccc2)cc1)c1ccc2c(c1)C(c1ccccc1)(c1ccccc1)c1ccccc1-2 ; Cc1ccccc1-c1cc2ccccc2cc1N(c1ccc(-c2ccc(C3CCCCC3)cc2)cc1)c1ccc2c(c1)C(c1ccccc1)(c1ccccc1)c1ccccc1-2 ; Cc1ccccc1-c1cc2ccccc2cc1N(c1ccc2c(c1)C(c1ccccc1)(c1ccccc1)c1ccccc1-2)c1ccc2ccccc2c1 ; Cc1ccccc1-c1cc2ccccc2cc1N(c1ccc2c(c1)C(c1ccccc1)(c1ccccc1)c1ccccc1-2)C1CCC(c2ccccc2)CC1]

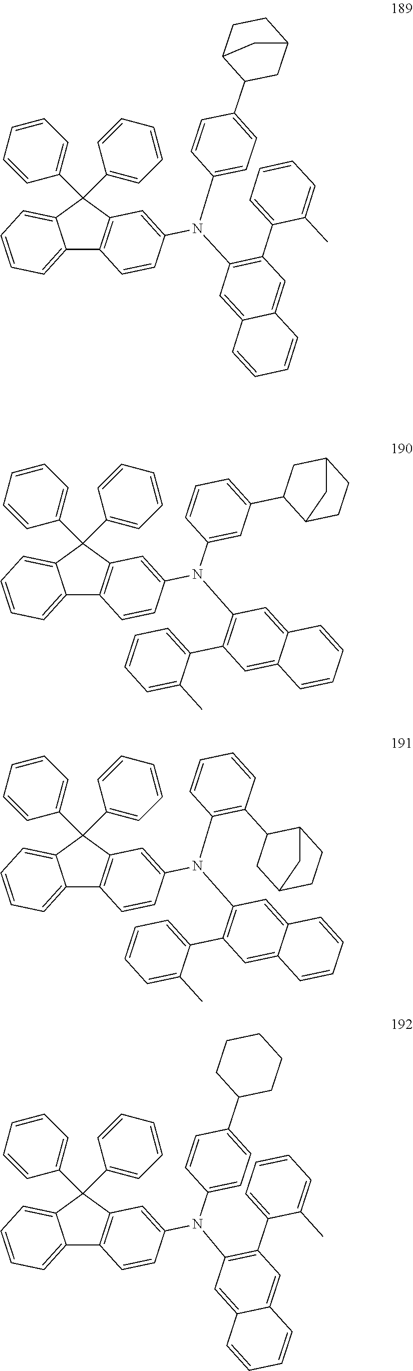

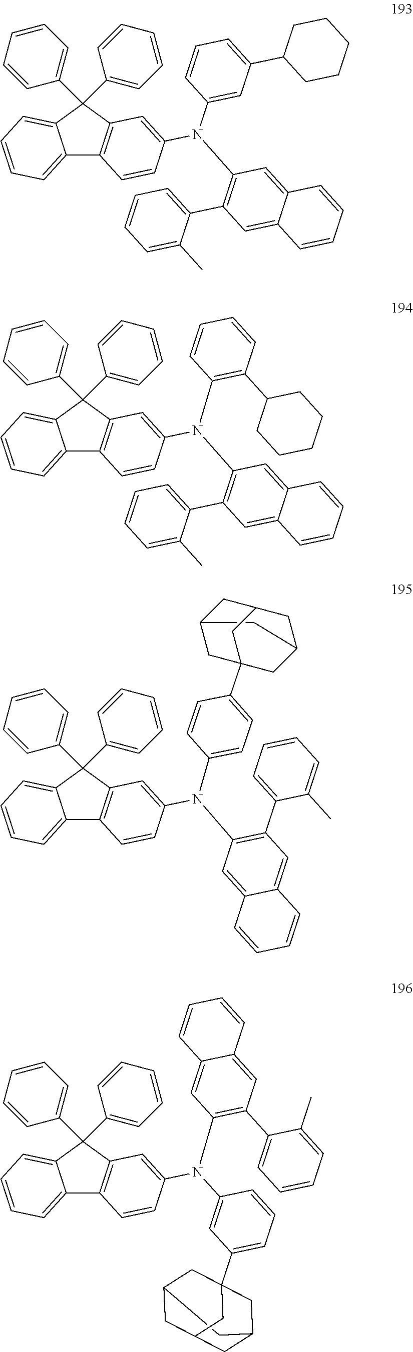

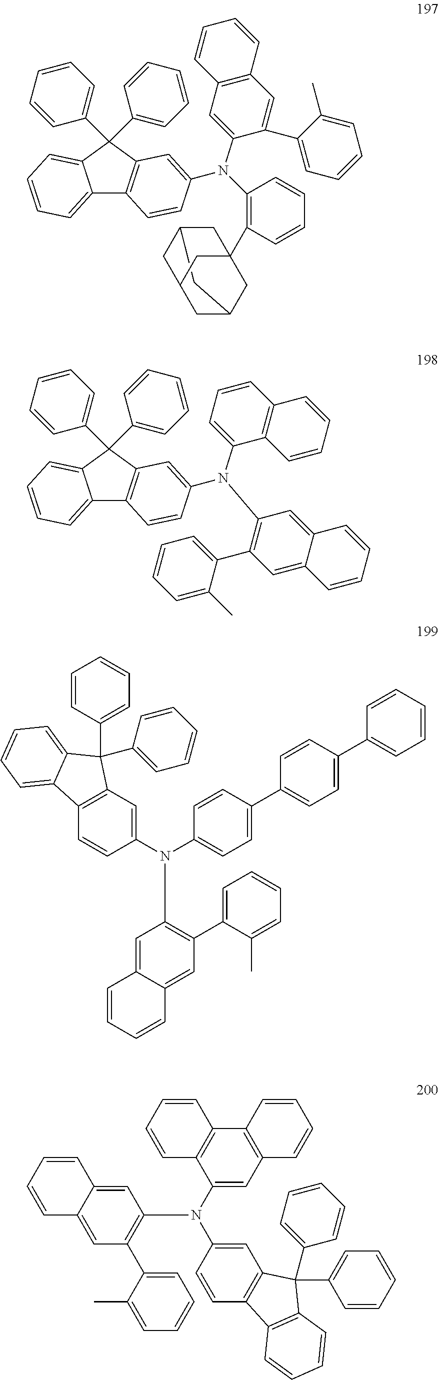 [SMILES: Cc1ccccc1-c1cc2ccccc2cc1N(c1ccc2c(c1)C(c1ccccc1)(c1ccccc1)c1ccccc1-2)c1ccccc1C12CC3CC(CC(C3)C1)C2 ; Cc1ccccc1-c1cc2ccccc2cc1N(c1ccc2c(c1)C(c1ccccc1)(c1ccccc1)c1ccccc1-2)c1cccc2ccccc12 ; Cc1ccccc1-c1cc2ccccc2cc1N(c1ccc(-c2ccc(-c3ccccc3)cc2)cc1)c1ccc2c(c1)C(c1ccccc1)(c1ccccc1)c1ccccc1-2 ; Cc1ccccc1-c1cc2ccccc2cc1N(c1ccc2c(c1)C(c1ccccc1)(c1ccccc1)c1ccccc1-2)c1cc2ccccc2c2ccccc12]

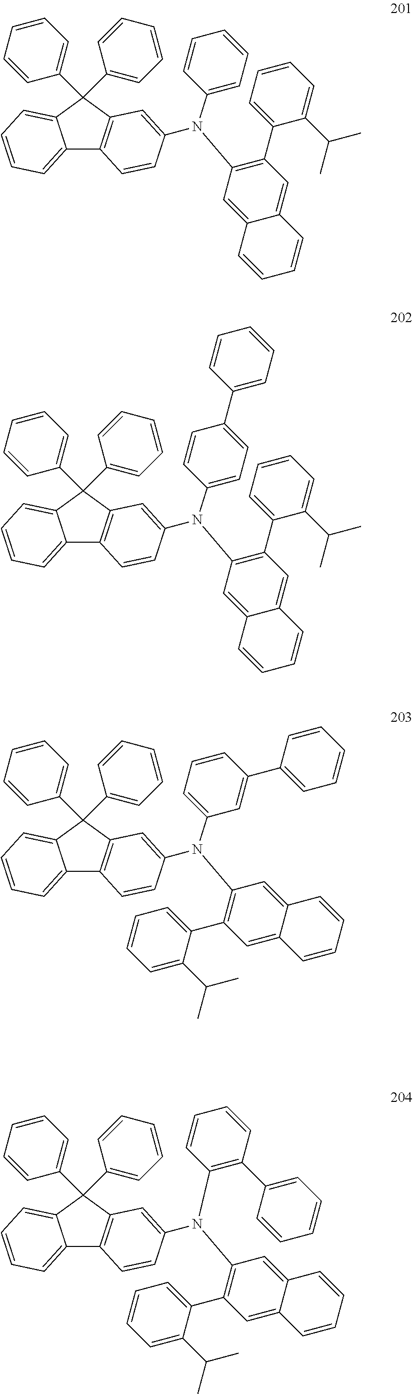 [SMILES: CC(C)c1ccccc1-c1cc2ccccc2cc1N(c1ccccc1)c1ccc2c(c1)C(c1ccccc1)(c1ccccc1)c1ccccc1-2 ; CC(C)c1ccccc1-c1cc2ccccc2cc1N(c1ccc(-c2ccccc2)cc1)c1ccc2c(c1)C(c1ccccc1)(c1ccccc1)c1ccccc1-2 ; CC(C)c1ccccc1-c1cc2ccccc2cc1N(c1cccc(-c2ccccc2)c1)c1ccc2c(c1)C(c1ccccc1)(c1ccccc1)c1ccccc1-2 ; CC(C)c1ccccc1-c1cc2ccccc2cc1N(c1ccc2c(c1)C(c1ccccc1)(c1ccccc1)c1ccccc1-2)c1ccccc1-c1ccccc1]

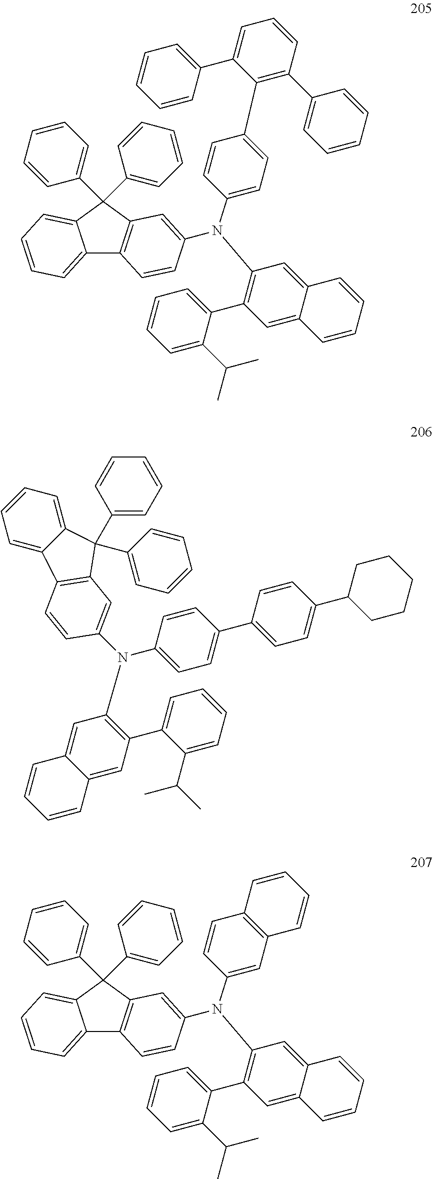 [SMILES: CC(C)c1ccccc1-c1cc2ccccc2cc1N(c1ccc(-c2c(-c3ccccc3)cccc2-c2ccccc2)cc1)c1ccc2c(c1)C(c1ccccc1)(c1ccccc1)c1ccccc1-2 ; CC(C)c1ccccc1-c1cc2ccccc2cc1N(c1ccc(-c2ccc(C3CCCCC3)cc2)cc1)c1ccc2c(c1)C(c1ccccc1)(c1ccccc1)c1ccccc1-2 ; CC(C)c1ccccc1-c1cc2ccccc2cc1N(c1ccc2c(c1)C(c1ccccc1)(c1ccccc1)c1ccccc1-2)c1ccc2ccccc2c1]

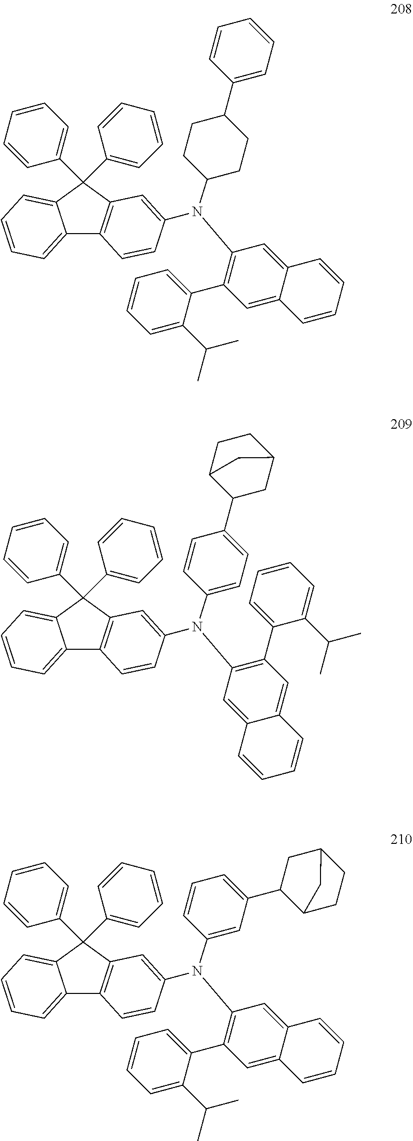 [SMILES: CC(C)c1ccccc1-c1cc2ccccc2cc1N(c1ccc2c(c1)C(c1ccccc1)(c1ccccc1)c1ccccc1-2)C1CCC(c2ccccc2)CC1 ; CC(C)c1ccccc1-c1cc2ccccc2cc1N(c1ccc(C2CC3CCC2C3)cc1)c1ccc2c(c1)C(c1ccccc1)(c1ccccc1)c1ccccc1-2 ; CC(C)c1ccccc1-c1cc2ccccc2cc1N(c1cccc(C2CC3CCC2C3)c1)c1ccc2c(c1)C(c1ccccc1)(c1ccccc1)c1ccccc1-2]

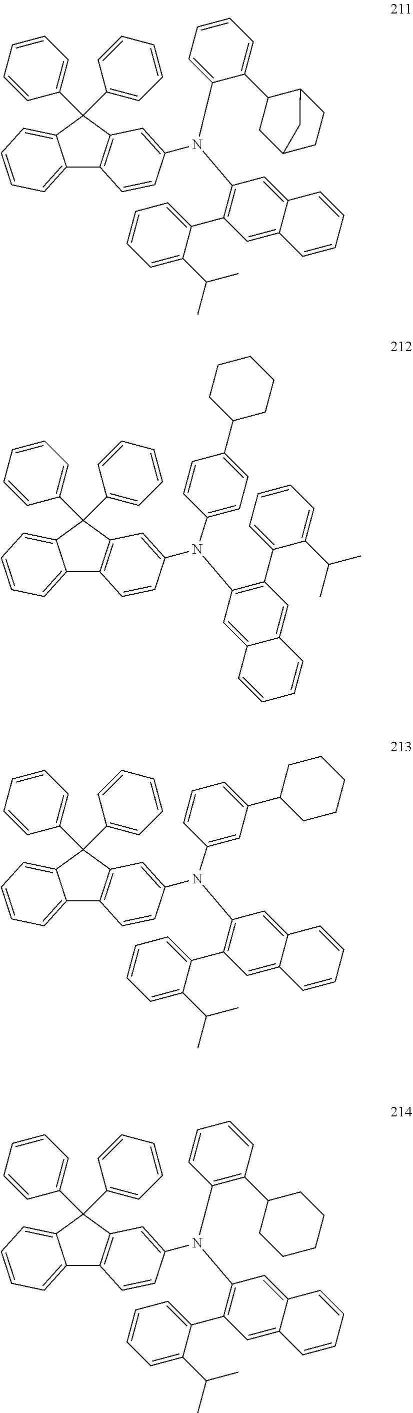 [SMILES: CC(C)c1ccccc1-c1cc2ccccc2cc1N(c1ccc2c(c1)C(c1ccccc1)(c1ccccc1)c1ccccc1-2)c1ccccc1C1CC2CCC1C2 ; CC(C)c1ccccc1-c1cc2ccccc2cc1N(c1ccc(C2CCCCC2)cc1)c1ccc2c(c1)C(c1ccccc1)(c1ccccc1)c1ccccc1-2 ; CC(C)c1ccccc1-c1cc2ccccc2cc1N(c1cccc(C2CCCCC2)c1)c1ccc2c(c1)C(c1ccccc1)(c1ccccc1)c1ccccc1-2 ; CC(C)c1ccccc1-c1cc2ccccc2cc1N(c1ccc2c(c1)C(c1ccccc1)(c1ccccc1)c1ccccc1-2)c1ccccc1C1CCCCC1]

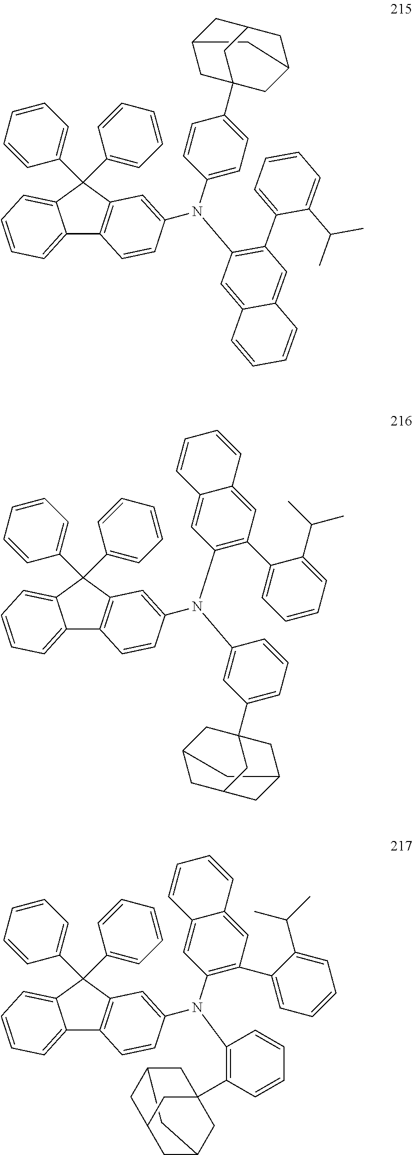 [SMILES: CC(C)c1ccccc1-c1cc2ccccc2cc1N(c1ccc(C23CC4CC(CC(C4)C2)C3)cc1)c1ccc2c(c1)C(c1ccccc1)(c1ccccc1)c1ccccc1-2 ; CC(C)c1ccccc1-c1cc2ccccc2cc1N(c1cccc(C23CC4CC(CC(C4)C2)C3)c1)c1ccc2c(c1)C(c1ccccc1)(c1ccccc1)c1ccccc1-2 ; CC(C)c1ccccc1-c1cc2ccccc2cc1N(c1ccc2c(c1)C(c1ccccc1)(c1ccccc1)c1ccccc1-2)c1ccccc1C12CC3CC(CC(C3)C1)C2]

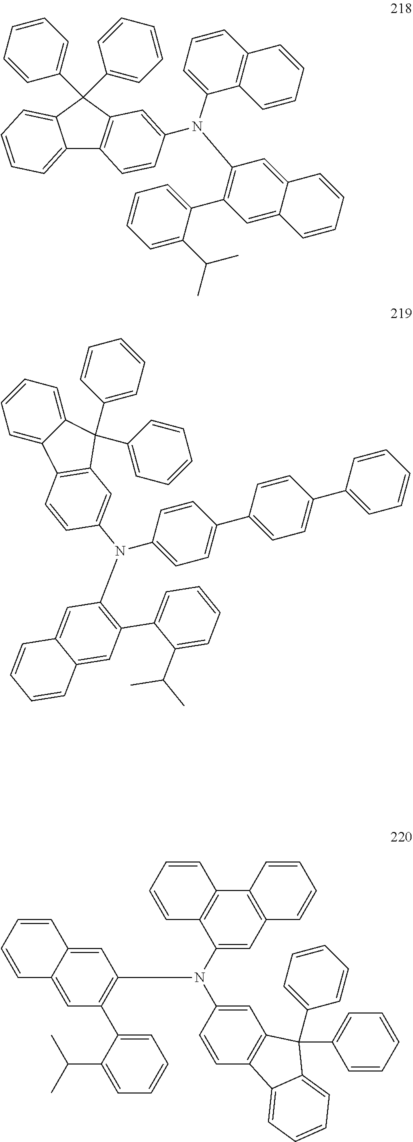 [SMILES: CC(C)c1ccccc1-c1cc2ccccc2cc1N(c1ccc2c(c1)C(c1ccccc1)(c1ccccc1)c1ccccc1-2)c1cccc2ccccc12 ; CC(C)c1ccccc1-c1cc2ccccc2cc1N(c1ccc(-c2ccc(-c3ccccc3)cc2)cc1)c1ccc2c(c1)C(c1ccccc1)(c1ccccc1)c1ccccc1-2 ; CC(C)c1ccccc1-c1cc2ccccc2cc1N(c1ccc2c(c1)C(c1ccccc1)(c1ccccc1)c1ccccc1-2)c1cc2ccccc2c2ccccc12]

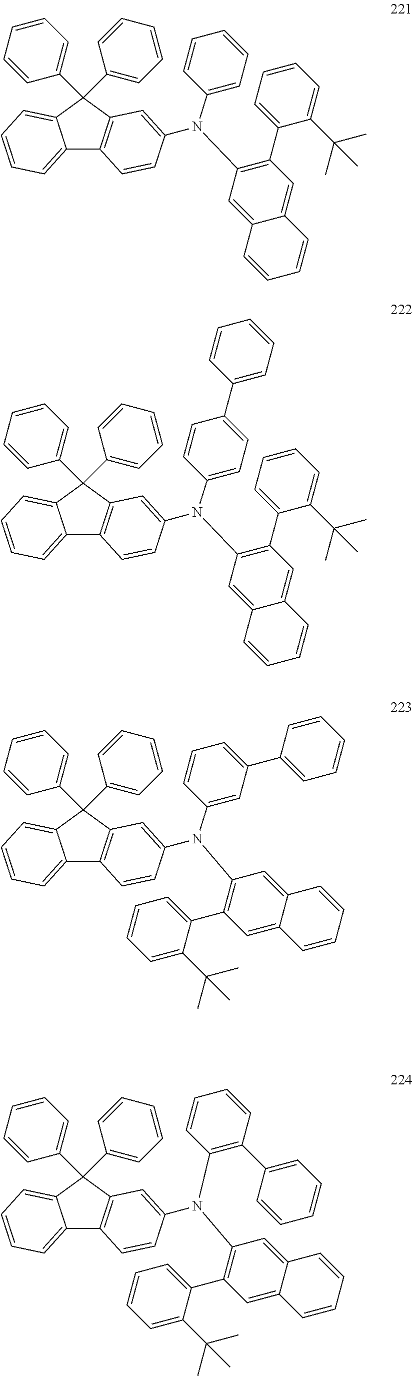

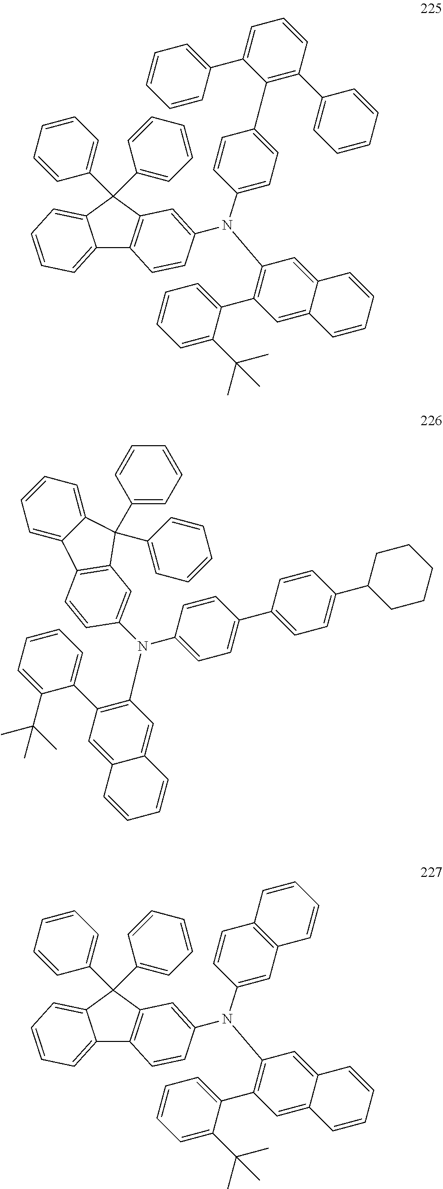 [SMILES: CC(C)(C)c1ccccc1-c1cc2ccccc2cc1N(c1ccc(-c2c(-c3ccccc3)cccc2-c2ccccc2)cc1)c1ccc2c(c1)C(c1ccccc1)(c1ccccc1)c1ccccc1-2 ; CC(C)(C)c1ccccc1-c1cc2ccccc2cc1N(c1ccc(-c2ccc(C3CCCCC3)cc2)cc1)c1ccc2c(c1)C(c1ccccc1)(c1ccccc1)c1ccccc1-2 ; CC(C)(C)c1ccccc1-c1cc2ccccc2cc1N(c1ccc2c(c1)C(c1ccccc1)(c1ccccc1)c1ccccc1-2)c1ccc2ccccc2c1]

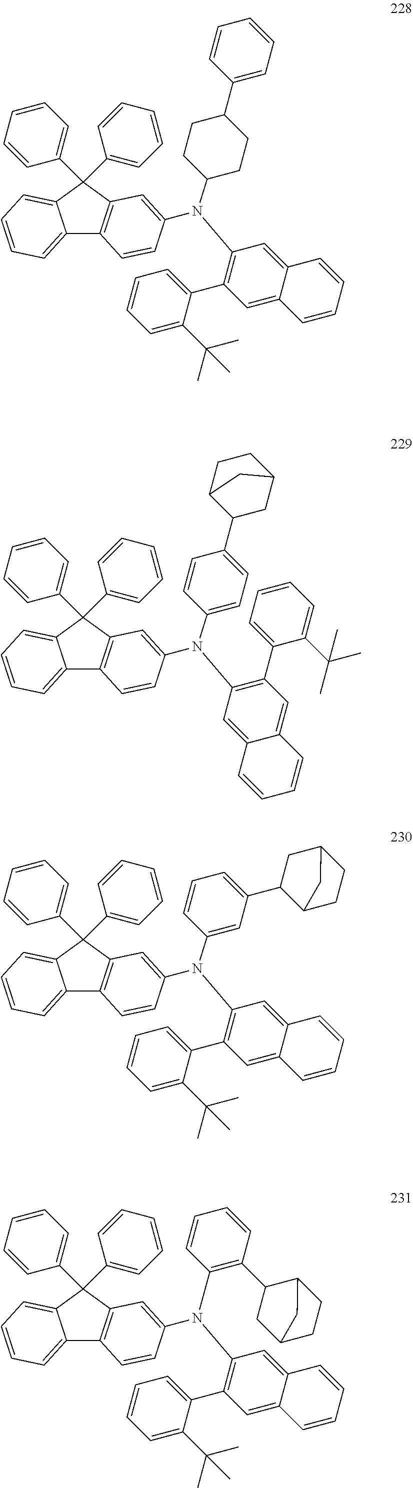 [SMILES: CC(C)(C)c1ccccc1-c1cc2ccccc2cc1N(c1ccc2c(c1)C(c1ccccc1)(c1ccccc1)c1ccccc1-2)C1CCC(c2ccccc2)CC1 ; CC(C)(C)c1ccccc1-c1cc2ccccc2cc1N(c1ccc(C2CC3CCC2C3)cc1)c1ccc2c(c1)C(c1ccccc1)(c1ccccc1)c1ccccc1-2 ; CC(C)(C)c1ccccc1-c1cc2ccccc2cc1N(c1cccc(C2CC3CCC2C3)c1)c1ccc2c(c1)C(c1ccccc1)(c1ccccc1)c1ccccc1-2 ; CC(C)(C)c1ccccc1-c1cc2ccccc2cc1N(c1ccc2c(c1)C(c1ccccc1)(c1ccccc1)c1ccccc1-2)c1ccccc1C1CC2CCC1C2]

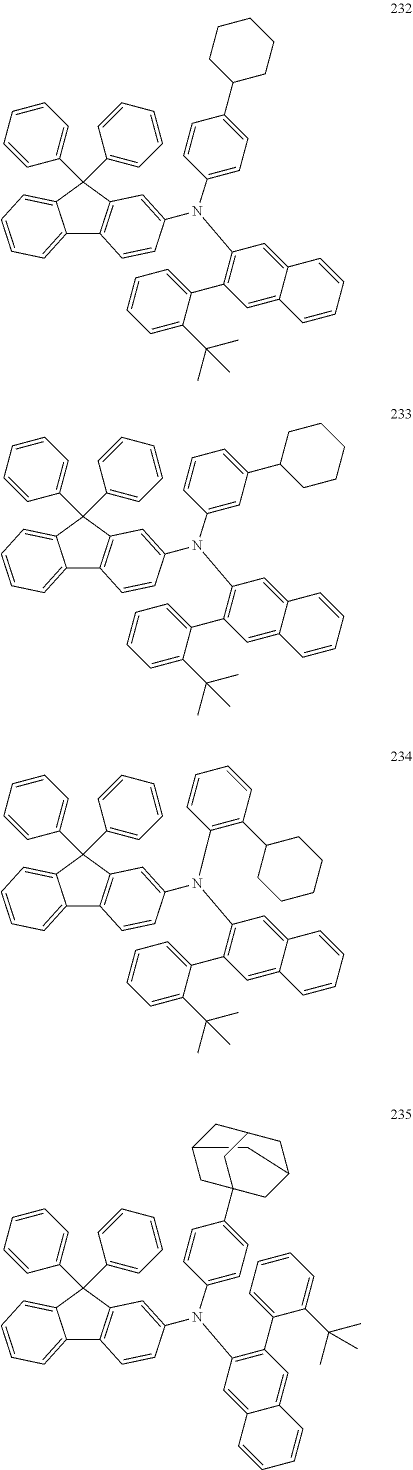 [SMILES: CC(C)(C)c1ccccc1-c1cc2ccccc2cc1N(c1ccc(C2CCCCC2)cc1)c1ccc2c(c1)C(c1ccccc1)(c1ccccc1)c1ccccc1-2 ; CC(C)(C)c1ccccc1-c1cc2ccccc2cc1N(c1cccc(C2CCCCC2)c1)c1ccc2c(c1)C(c1ccccc1)(c1ccccc1)c1ccccc1-2 ; CC(C)(C)c1ccccc1-c1cc2ccccc2cc1N(c1ccc2c(c1)C(c1ccccc1)(c1ccccc1)c1ccccc1-2)c1ccccc1C1CCCCC1 ; CC(C)(C)c1ccccc1-c1cc2ccccc2cc1N(c1ccc(C23CC4CC(CC(C4)C2)C3)cc1)c1ccc2c(c1)C(c1ccccc1)(c1ccccc1)c1ccccc1-2]

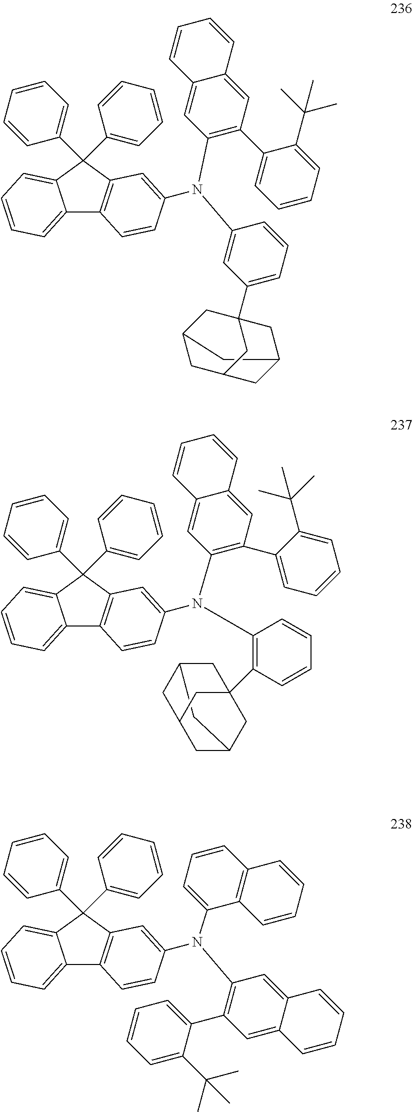 [SMILES: CC(C)(C)c1ccccc1-c1cc2ccccc2cc1N(c1cccc(C23CC4CC(CC(C4)C2)C3)c1)c1ccc2c(c1)C(c1ccccc1)(c1ccccc1)c1ccccc1-2 ; CC(C)(C)c1ccccc1-c1cc2ccccc2cc1N(c1ccc2c(c1)C(c1ccccc1)(c1ccccc1)c1ccccc1-2)c1ccccc1C12CC3CC(CC(C3)C1)C2 ; CC(C)(C)c1ccccc1-c1cc2ccccc2cc1N(c1ccc2c(c1)C(c1ccccc1)(c1ccccc1)c1ccccc1-2)c1cccc2ccccc12]

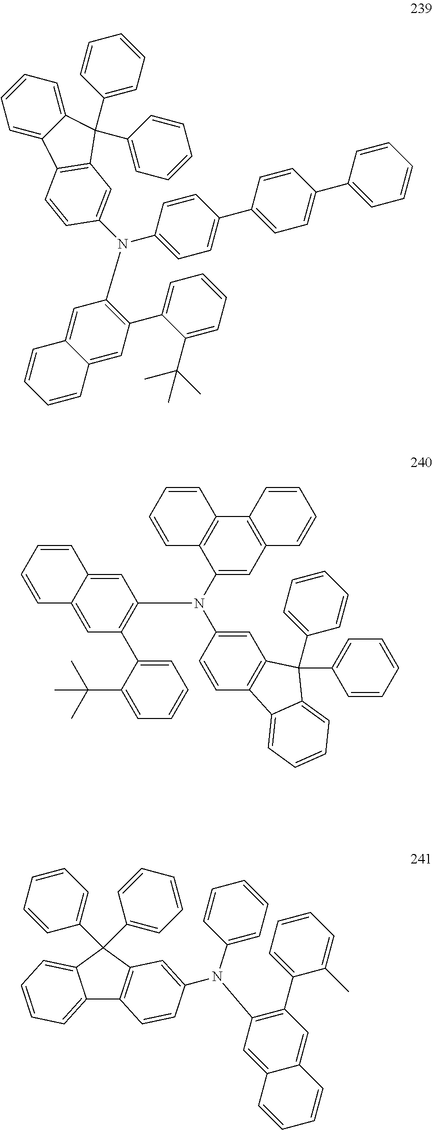 [SMILES: CC(C)(C)c1ccccc1-c1cc2ccccc2cc1N(c1ccc(-c2ccc(-c3ccccc3)cc2)cc1)c1ccc2c(c1)C(c1ccccc1)(c1ccccc1)c1ccccc1-2 ; CC(C)(C)c1ccccc1-c1cc2ccccc2cc1N(c1ccc2c(c1)C(c1ccccc1)(c1ccccc1)c1ccccc1-2)c1cc2ccccc2c2ccccc12 ; Cc1ccccc1-c1cc2ccccc2cc1N(c1ccccc1)c1ccc2c(c1)C(c1ccccc1)(c1ccccc1)c1ccccc1-2]

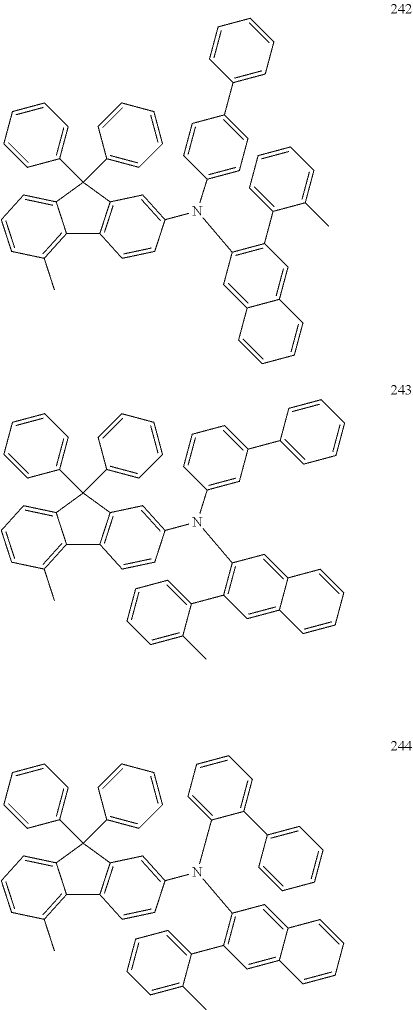 [SMILES: Cc1ccccc1-c1cc2ccccc2cc1N(c1ccc(-c2ccccc2)cc1)c1ccc2c(c1)C(c1ccccc1)(c1ccccc1)c1cccc(C)c1-2 ; Cc1ccccc1-c1cc2ccccc2cc1N(c1cccc(-c2ccccc2)c1)c1ccc2c(c1)C(c1ccccc1)(c1ccccc1)c1cccc(C)c1-2 ; Cc1ccccc1-c1cc2ccccc2cc1N(c1ccc2c(c1)C(c1ccccc1)(c1ccccc1)c1cccc(C)c1-2)c1ccccc1-c1ccccc1]

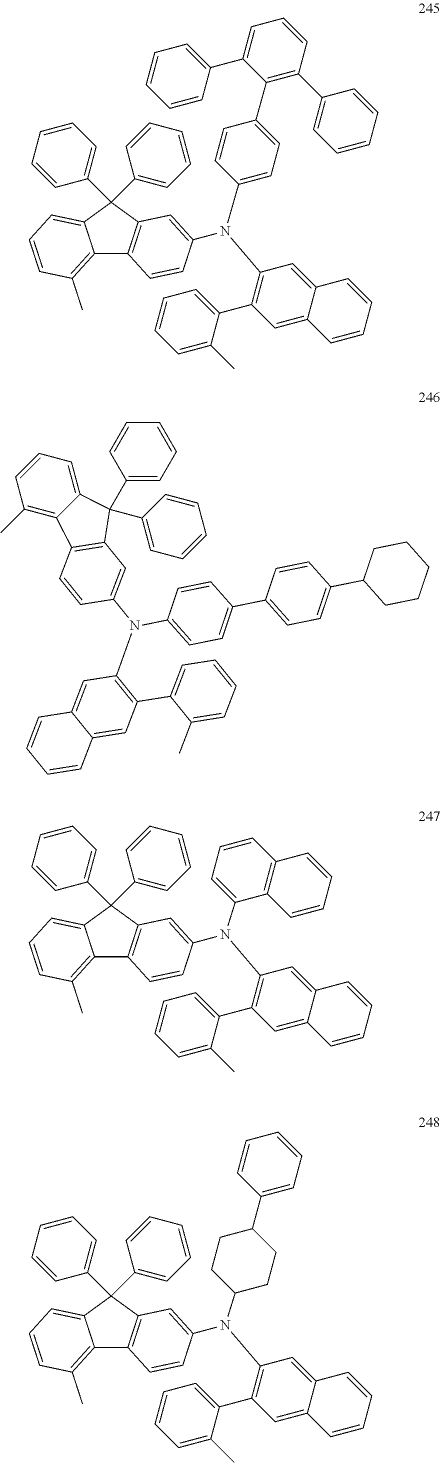 [SMILES: Cc1ccccc1-c1cc2ccccc2cc1N(c1ccc(-c2c(-c3ccccc3)cccc2-c2ccccc2)cc1)c1ccc2c(c1)C(c1ccccc1)(c1ccccc1)c1cccc(C)c1-2 ; Cc1ccccc1-c1cc2ccccc2cc1N(c1ccc(-c2ccc(C3CCCCC3)cc2)cc1)c1ccc2c(c1)C(c1ccccc1)(c1ccccc1)c1cccc(C)c1-2 ; Cc1ccccc1-c1cc2ccccc2cc1N(c1ccc2c(c1)C(c1ccccc1)(c1ccccc1)c1cccc(C)c1-2)c1cccc2ccccc12 ; Cc1ccccc1-c1cc2ccccc2cc1N(c1ccc2c(c1)C(c1ccccc1)(c1ccccc1)c1cccc(C)c1-2)C1CCC(c2ccccc2)CC1]

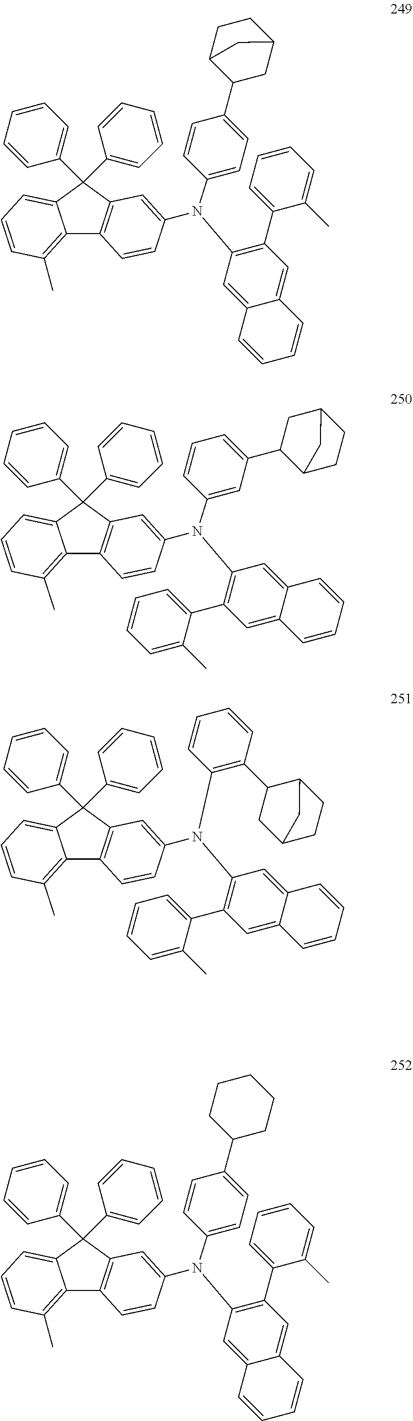

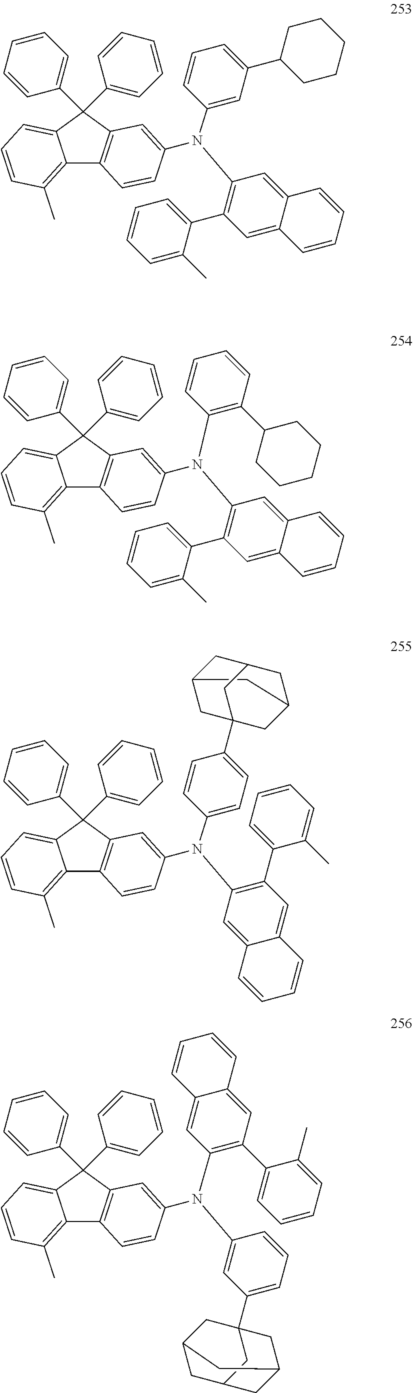 [SMILES: Cc1ccccc1-c1cc2ccccc2cc1N(c1cccc(C2CCCCC2)c1)c1ccc2c(c1)C(c1ccccc1)(c1ccccc1)c1cccc(C)c1-2 ; Cc1ccccc1-c1cc2ccccc2cc1N(c1ccc2c(c1)C(c1ccccc1)(c1ccccc1)c1cccc(C)c1-2)c1ccccc1C1CCCCC1 ; Cc1ccccc1-c1cc2ccccc2cc1N(c1ccc(C23CC4CC(CC(C4)C2)C3)cc1)c1ccc2c(c1)C(c1ccccc1)(c1ccccc1)c1cccc(C)c1-2 ; Cc1ccccc1-c1cc2ccccc2cc1N(c1cccc(C23CC4CC(CC(C4)C2)C3)c1)c1ccc2c(c1)C(c1ccccc1)(c1ccccc1)c1cccc(C)c1-2]

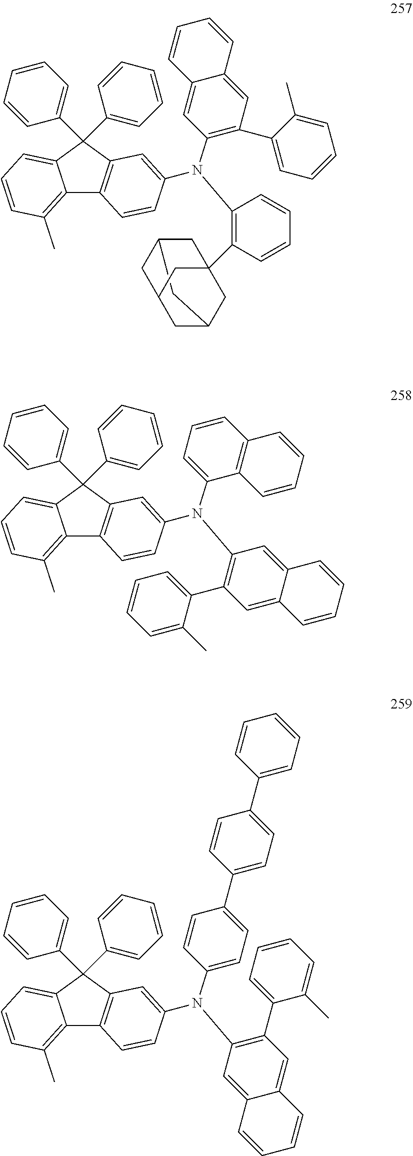 [SMILES: Cc1ccccc1-c1cc2ccccc2cc1N(c1ccc2c(c1)C(c1ccccc1)(c1ccccc1)c1cccc(C)c1-2)c1ccccc1C12CC3CC(CC(C3)C1)C2 ; Cc1ccccc1-c1cc2ccccc2cc1N(c1ccc2c(c1)C(c1ccccc1)(c1ccccc1)c1cccc(C)c1-2)c1cccc2ccccc12 ; Cc1ccccc1-c1cc2ccccc2cc1N(c1ccc(-c2ccc(-c3ccccc3)cc2)cc1)c1ccc2c(c1)C(c1ccccc1)(c1ccccc1)c1cccc(C)c1-2]

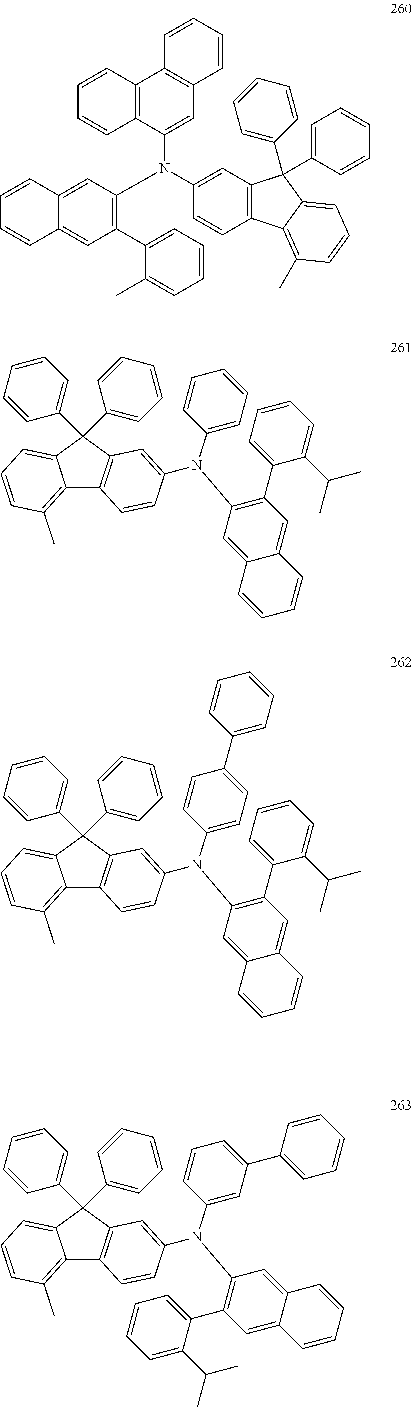 [SMILES: Cc1ccccc1-c1cc2ccccc2cc1N(c1ccc2c(c1)C(c1ccccc1)(c1ccccc1)c1cccc(C)c1-2)c1cc2ccccc2c2ccccc12 ; Cc1cccc2c1-c1ccc(N(c3ccccc3)c3cc4ccccc4cc3-c3ccccc3C(C)C)cc1C2(c1ccccc1)c1ccccc1 ; Cc1cccc2c1-c1ccc(N(c3ccc(-c4ccccc4)cc3)c3cc4ccccc4cc3-c3ccccc3C(C)C)cc1C2(c1ccccc1)c1ccccc1 ; Cc1cccc2c1-c1ccc(N(c3cccc(-c4ccccc4)c3)c3cc4ccccc4cc3-c3ccccc3C(C)C)cc1C2(c1ccccc1)c1ccccc1]

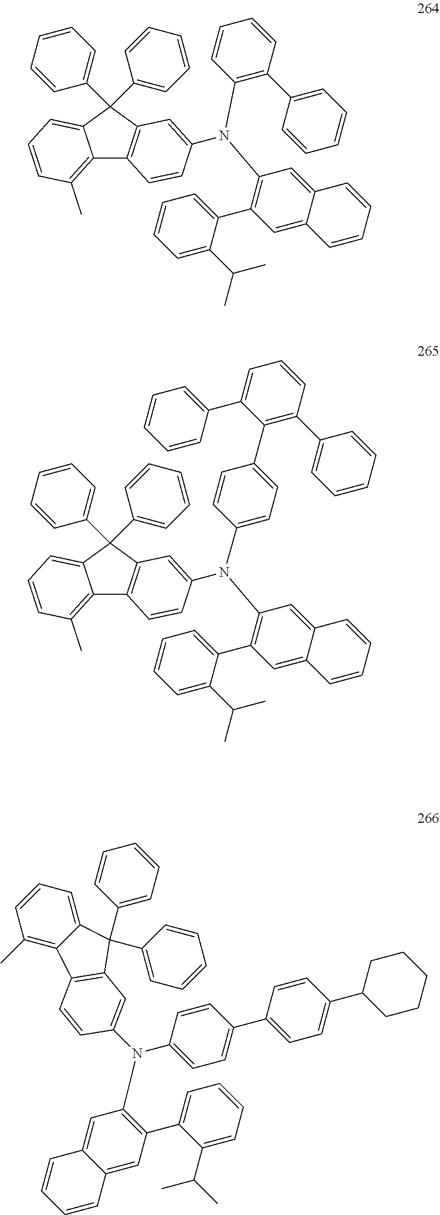 [SMILES: Cc1cccc2c1-c1ccc(N(c3ccccc3-c3ccccc3)c3cc4ccccc4cc3-c3ccccc3C(C)C)cc1C2(c1ccccc1)c1ccccc1 ; Cc1cccc2c1-c1ccc(N(c3ccc(-c4c(-c5ccccc5)cccc4-c4ccccc4)cc3)c3cc4ccccc4cc3-c3ccccc3C(C)C)cc1C2(c1ccccc1)c1ccccc1 ; Cc1cccc2c1-c1ccc(N(c3ccc(-c4ccc(C5CCCCC5)cc4)cc3)c3cc4ccccc4cc3-c3ccccc3C(C)C)cc1C2(c1ccccc1)c1ccccc1]

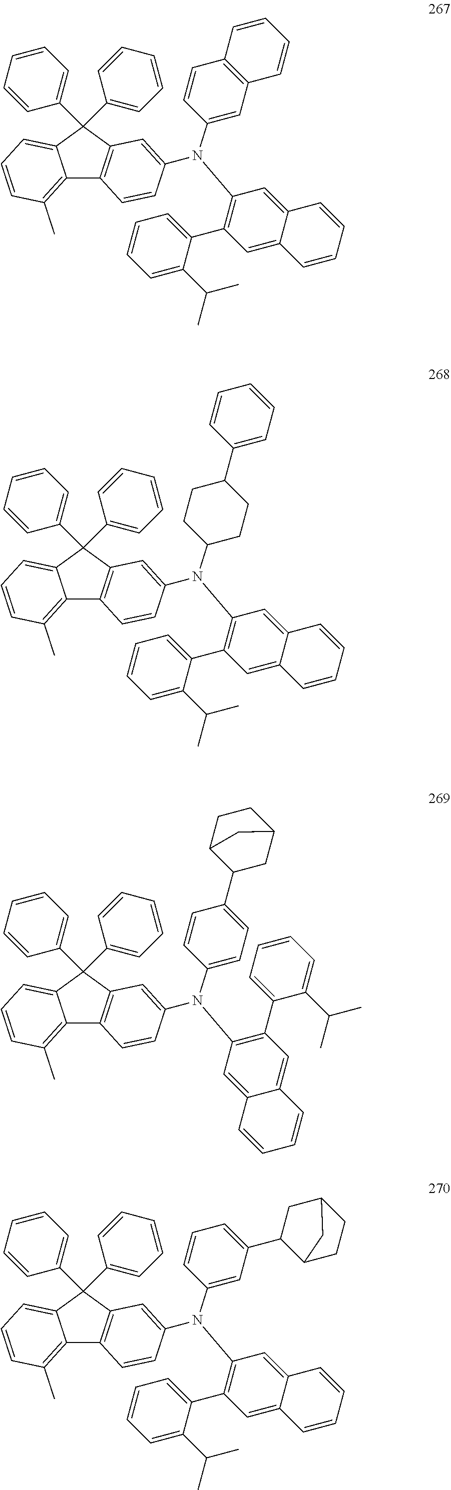 [SMILES: Cc1cccc2c1-c1ccc(N(c3ccc4ccccc4c3)c3cc4ccccc4cc3-c3ccccc3C(C)C)cc1C2(c1ccccc1)c1ccccc1 ; Cc1cccc2c1-c1ccc(N(c3cc4ccccc4cc3-c3ccccc3C(C)C)C3CCC(c4ccccc4)CC3)cc1C2(c1ccccc1)c1ccccc1 ; Cc1cccc2c1-c1ccc(N(c3ccc(C4CC5CCC4C5)cc3)c3cc4ccccc4cc3-c3ccccc3C(C)C)cc1C2(c1ccccc1)c1ccccc1 ; Cc1cccc2c1-c1ccc(N(c3cccc(C4CC5CCC4C5)c3)c3cc4ccccc4cc3-c3ccccc3C(C)C)cc1C2(c1ccccc1)c1ccccc1]

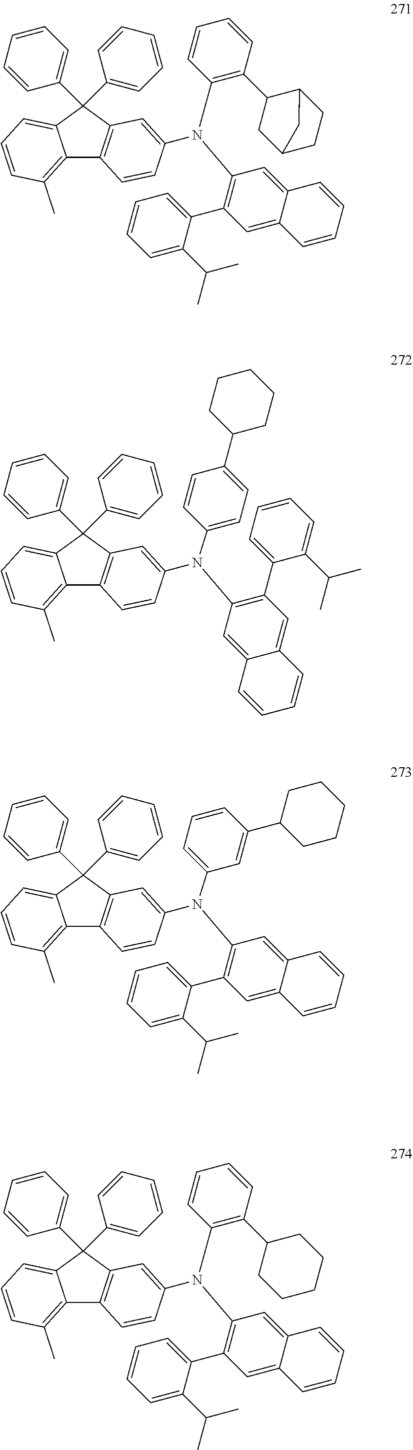 [SMILES: Cc1cccc2c1-c1ccc(N(c3cc4ccccc4cc3-c3ccccc3C(C)C)c3ccccc3C3CC4CCC3C4)cc1C2(c1ccccc1)c1ccccc1 ; Cc1cccc2c1-c1ccc(N(c3ccc(C4CCCCC4)cc3)c3cc4ccccc4cc3-c3ccccc3C(C)C)cc1C2(c1ccccc1)c1ccccc1 ; Cc1cccc2c1-c1ccc(N(c3cccc(C4CCCCC4)c3)c3cc4ccccc4cc3-c3ccccc3C(C)C)cc1C2(c1ccccc1)c1ccccc1 ; Cc1cccc2c1-c1ccc(N(c3cc4ccccc4cc3-c3ccccc3C(C)C)c3ccccc3C3CCCCC3)cc1C2(c1ccccc1)c1ccccc1]

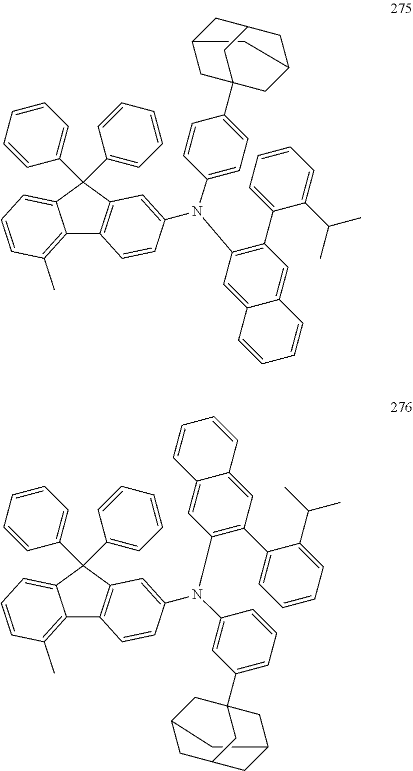 [SMILES: Cc1cccc2c1-c1ccc(N(c3ccc(C45CC6CC(CC(C6)C4)C5)cc3)c3cc4ccccc4cc3-c3ccccc3C(C)C)cc1C2(c1ccccc1)c1ccccc1 ; Cc1cccc2c1-c1ccc(N(c3cccc(C45CC6CC(CC(C6)C4)C5)c3)c3cc4ccccc4cc3-c3ccccc3C(C)C)cc1C2(c1ccccc1)c1ccccc1]

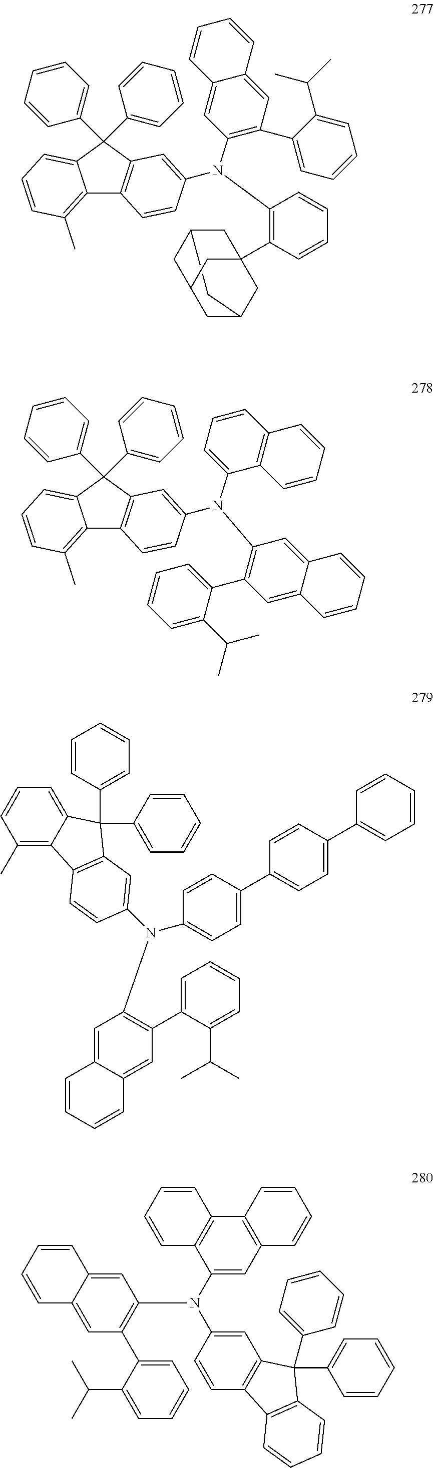 [SMILES: Cc1cccc2c1-c1ccc(N(c3cc4ccccc4cc3-c3ccccc3C(C)C)c3ccccc3C34CC5CC(CC(C5)C3)C4)cc1C2(c1ccccc1)c1ccccc1 ; Cc1cccc2c1-c1ccc(N(c3cc4ccccc4cc3-c3ccccc3C(C)C)c3cccc4ccccc34)cc1C2(c1ccccc1)c1ccccc1 ; Cc1cccc2c1-c1ccc(N(c3ccc(-c4ccc(-c5ccccc5)cc4)cc3)c3cc4ccccc4cc3-c3ccccc3C(C)C)cc1C2(c1ccccc1)c1ccccc1 ; CC(C)c1ccccc1-c1cc2ccccc2cc1N(c1ccc2c(c1)C(c1ccccc1)(c1ccccc1)c1ccccc1-2)c1cc2ccccc2c2ccccc12]

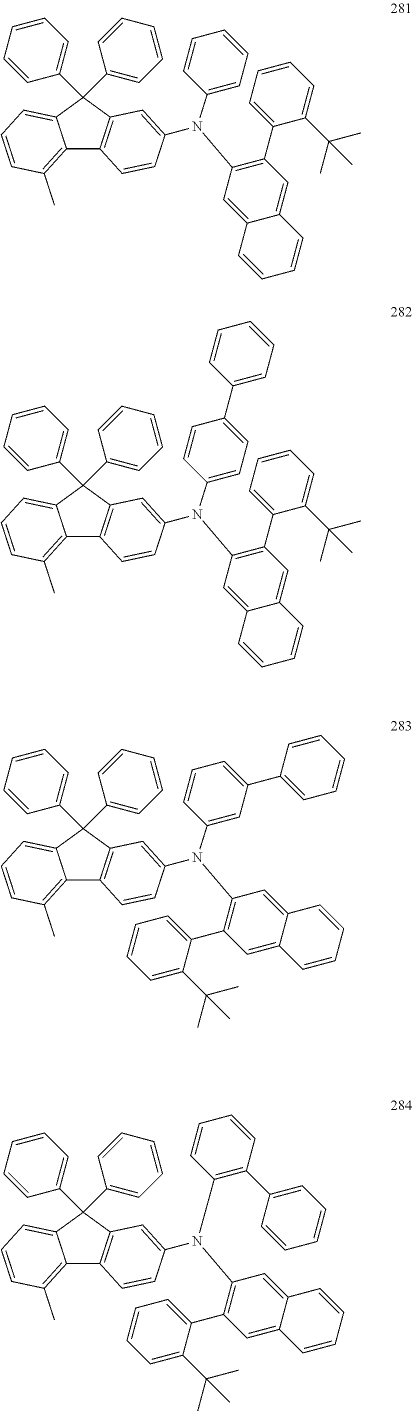

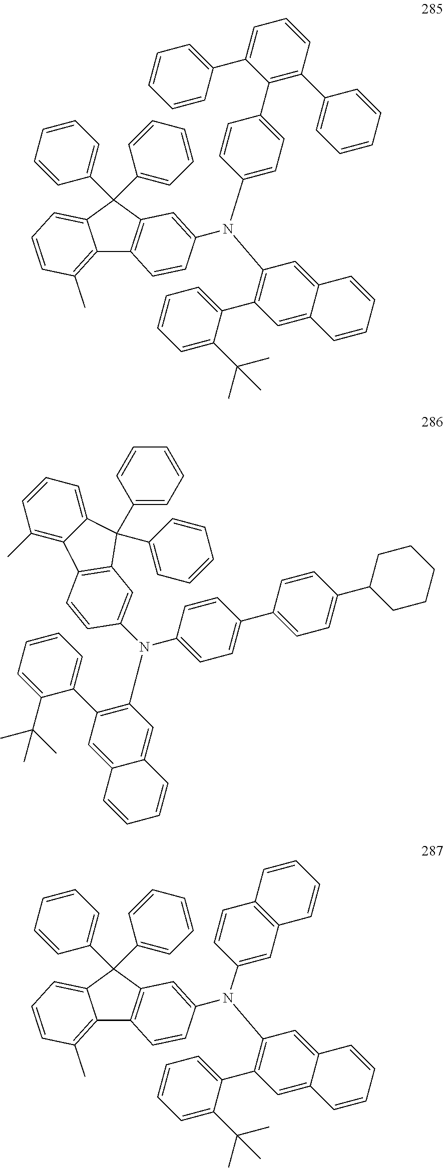 [SMILES: Cc1cccc2c1-c1ccc(N(c3ccc(-c4c(-c5ccccc5)cccc4-c4ccccc4)cc3)c3cc4ccccc4cc3-c3ccccc3C(C)(C)C)cc1C2(c1ccccc1)c1ccccc1 ; Cc1cccc2c1-c1ccc(N(c3ccc(-c4ccc(C5CCCCC5)cc4)cc3)c3cc4ccccc4cc3-c3ccccc3C(C)(C)C)cc1C2(c1ccccc1)c1ccccc1 ; Cc1cccc2c1-c1ccc(N(c3ccc4ccccc4c3)c3cc4ccccc4cc3-c3ccccc3C(C)(C)C)cc1C2(c1ccccc1)c1ccccc1]

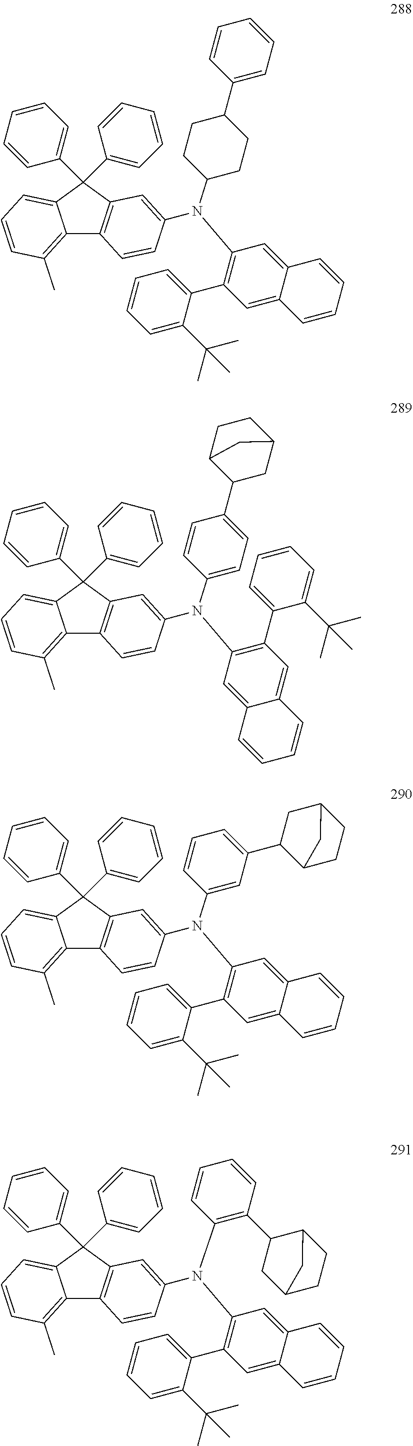 [SMILES: Cc1cccc2c1-c1ccc(N(c3cc4ccccc4cc3-c3ccccc3C(C)(C)C)C3CCC(c4ccccc4)CC3)cc1C2(c1ccccc1)c1ccccc1 ; Cc1cccc2c1-c1ccc(N(c3ccc(C4CC5CCC4C5)cc3)c3cc4ccccc4cc3-c3ccccc3C(C)(C)C)cc1C2(c1ccccc1)c1ccccc1 ; Cc1cccc2c1-c1ccc(N(c3cccc(C4CC5CCC4C5)c3)c3cc4ccccc4cc3-c3ccccc3C(C)(C)C)cc1C2(c1ccccc1)c1ccccc1 ; Cc1cccc2c1-c1ccc(N(c3cc4ccccc4cc3-c3ccccc3C(C)(C)C)c3ccccc3C3CC4CCC3C4)cc1C2(c1ccccc1)c1ccccc1]

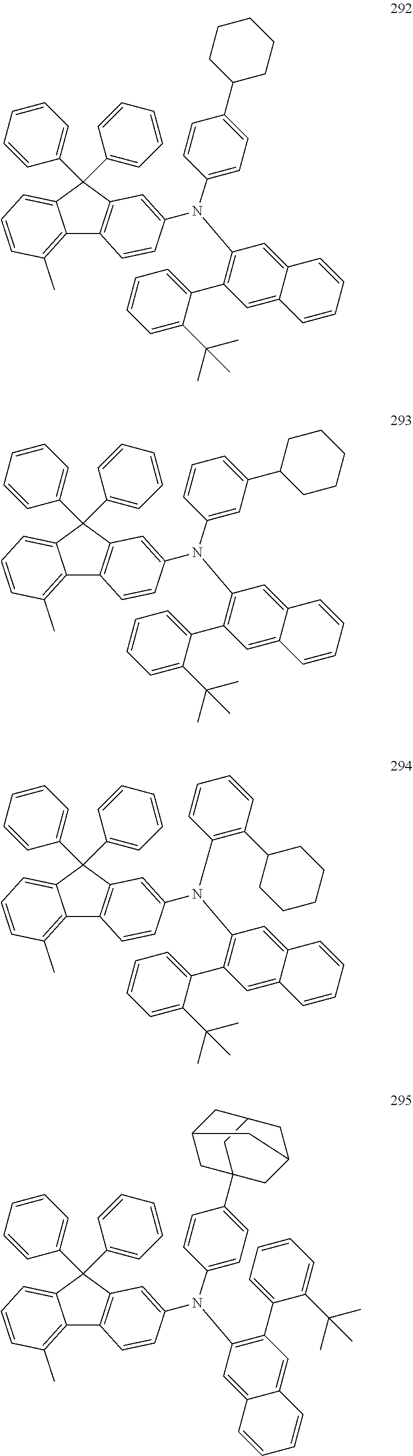 [SMILES: Cc1cccc2c1-c1ccc(N(c3ccc(C4CCCCC4)cc3)c3cc4ccccc4cc3-c3ccccc3C(C)(C)C)cc1C2(c1ccccc1)c1ccccc1 ; Cc1cccc2c1-c1ccc(N(c3cccc(C4CCCCC4)c3)c3cc4ccccc4cc3-c3ccccc3C(C)(C)C)cc1C2(c1ccccc1)c1ccccc1 ; Cc1cccc2c1-c1ccc(N(c3cc4ccccc4cc3-c3ccccc3C(C)(C)C)c3ccccc3C3CCCCC3)cc1C2(c1ccccc1)c1ccccc1 ; Cc1cccc2c1-c1ccc(N(c3ccc(C45CC6CC(CC(C6)C4)C5)cc3)c3cc4ccccc4cc3-c3ccccc3C(C)(C)C)cc1C2(c1ccccc1)c1ccccc1]

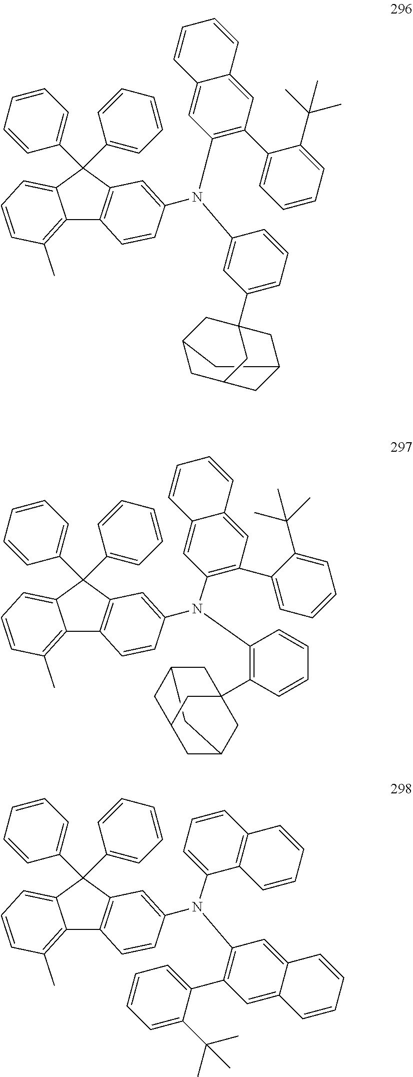 [SMILES: Cc1cccc2c1-c1ccc(N(c3cccc(C45CC6CC(CC(C6)C4)C5)c3)c3cc4ccccc4cc3-c3ccccc3C(C)(C)C)cc1C2(c1ccccc1)c1ccccc1 ; Cc1cccc2c1-c1ccc(N(c3cc4ccccc4cc3-c3ccccc3C(C)(C)C)c3ccccc3C34CC5CC(CC(C5)C3)C4)cc1C2(c1ccccc1)c1ccccc1 ; Cc1cccc2c1-c1ccc(N(c3cc4ccccc4cc3-c3ccccc3C(C)(C)C)c3cccc4ccccc34)cc1C2(c1ccccc1)c1ccccc1]

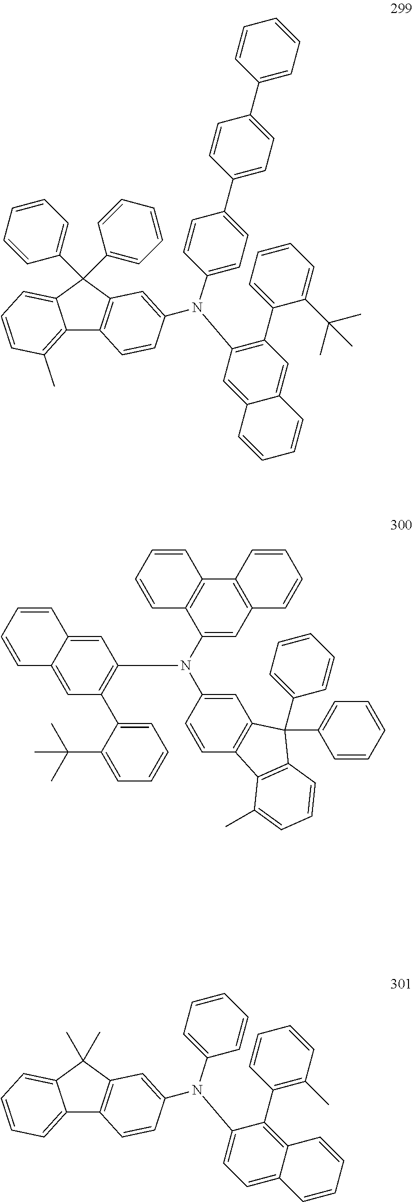 [SMILES: Cc1cccc2c1-c1ccc(N(c3ccc(-c4ccc(-c5ccccc5)cc4)cc3)c3cc4ccccc4cc3-c3ccccc3C(C)(C)C)cc1C2(c1ccccc1)c1ccccc1 ; Cc1cccc2c1-c1ccc(N(c3cc4ccccc4cc3-c3ccccc3C(C)(C)C)c3cc4ccccc4c4ccccc34)cc1C2(c1ccccc1)c1ccccc1 ; Cc1ccccc1-c1c(N(c2ccccc2)c2ccc3c(c2)C(C)(C)c2ccccc2-3)ccc2ccccc12]

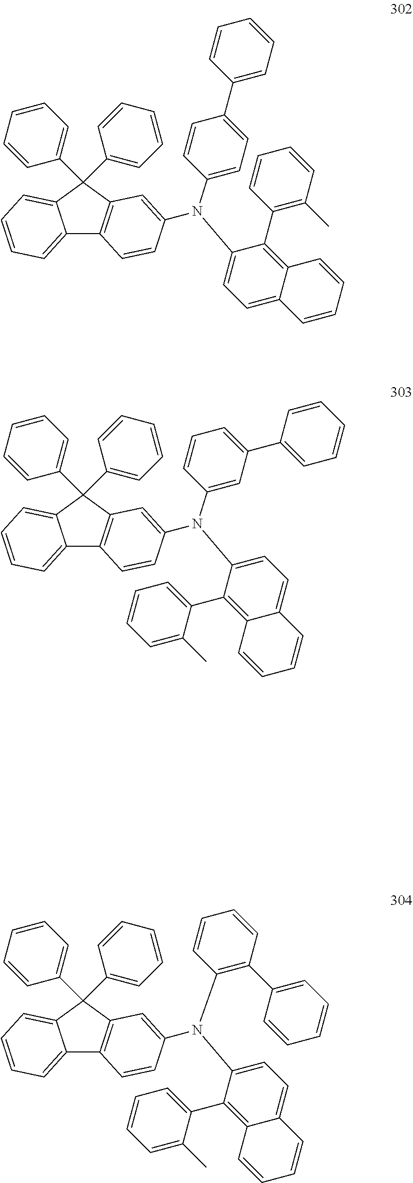 [SMILES: Cc1ccccc1-c1c(N(c2ccc(-c3ccccc3)cc2)c2ccc3c(c2)C(c2ccccc2)(c2ccccc2)c2ccccc2-3)ccc2ccccc12 ; Cc1ccccc1-c1c(N(c2cccc(-c3ccccc3)c2)c2ccc3c(c2)C(c2ccccc2)(c2ccccc2)c2ccccc2-3)ccc2ccccc12 ; Cc1ccccc1-c1c(N(c2ccc3c(c2)C(c2ccccc2)(c2ccccc2)c2ccccc2-3)c2ccccc2-c2ccccc2)ccc2ccccc12]

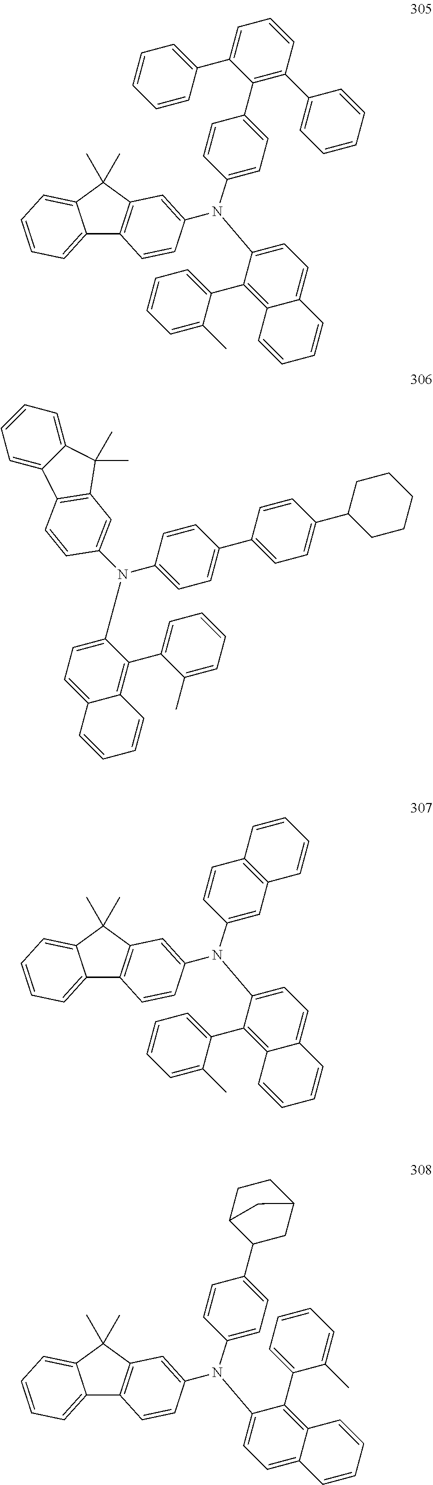 [SMILES: Cc1ccccc1-c1c(N(c2ccc(-c3c(-c4ccccc4)cccc3-c3ccccc3)cc2)c2ccc3c(c2)C(C)(C)c2ccccc2-3)ccc2ccccc12 ; Cc1ccccc1-c1c(N(c2ccc(-c3ccc(C4CCCCC4)cc3)cc2)c2ccc3c(c2)C(C)(C)c2ccccc2-3)ccc2ccccc12 ; Cc1ccccc1-c1c(N(c2ccc3c(c2)C(C)(C)c2ccccc2-3)c2ccc3ccccc3c2)ccc2ccccc12 ; Cc1ccccc1-c1c(N(c2ccc(C3CC4CCC3C4)cc2)c2ccc3c(c2)C(C)(C)c2ccccc2-3)ccc2ccccc12]

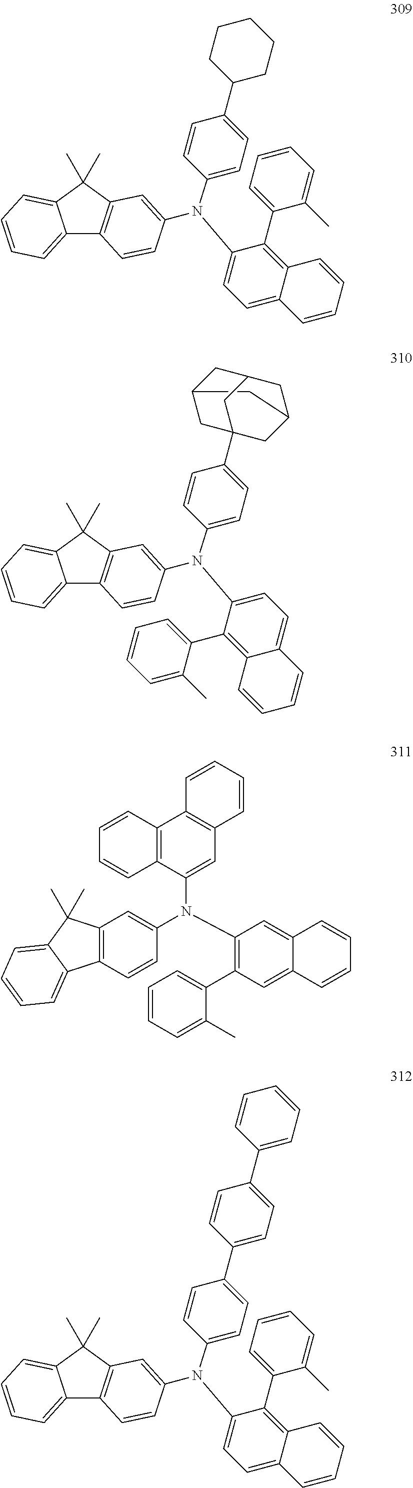 [SMILES: Cc1ccccc1-c1c(N(c2ccc(C3CCCCC3)cc2)c2ccc3c(c2)C(C)(C)c2ccccc2-3)ccc2ccccc12 ; Cc1ccccc1-c1c(N(c2ccc(C34CC5CC(CC(C5)C3)C4)cc2)c2ccc3c(c2)C(C)(C)c2ccccc2-3)ccc2ccccc12 ; Cc1ccccc1-c1cc2ccccc2cc1N(c1ccc2c(c1)C(C)(C)c1ccccc1-2)c1cc2ccccc2c2ccccc12 ; Cc1ccccc1-c1c(N(c2ccc(-c3ccc(-c4ccccc4)cc3)cc2)c2ccc3c(c2)C(C)(C)c2ccccc2-3)ccc2ccccc12]

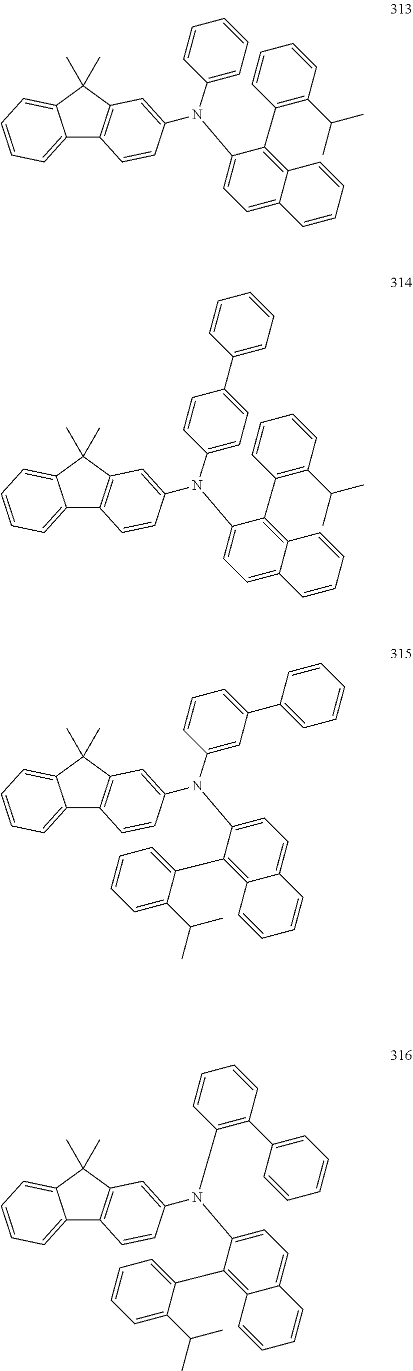 [SMILES: CC(C)c1ccccc1-c1c(N(c2ccccc2)c2ccc3c(c2)C(C)(C)c2ccccc2-3)ccc2ccccc12 ; CC(C)c1ccccc1-c1c(N(c2ccc(-c3ccccc3)cc2)c2ccc3c(c2)C(C)(C)c2ccccc2-3)ccc2ccccc12 ; CC(C)c1ccccc1-c1c(N(c2cccc(-c3ccccc3)c2)c2ccc3c(c2)C(C)(C)c2ccccc2-3)ccc2ccccc12 ; CC(C)c1ccccc1-c1c(N(c2ccc3c(c2)C(C)(C)c2ccccc2-3)c2ccccc2-c2ccccc2)ccc2ccccc12]

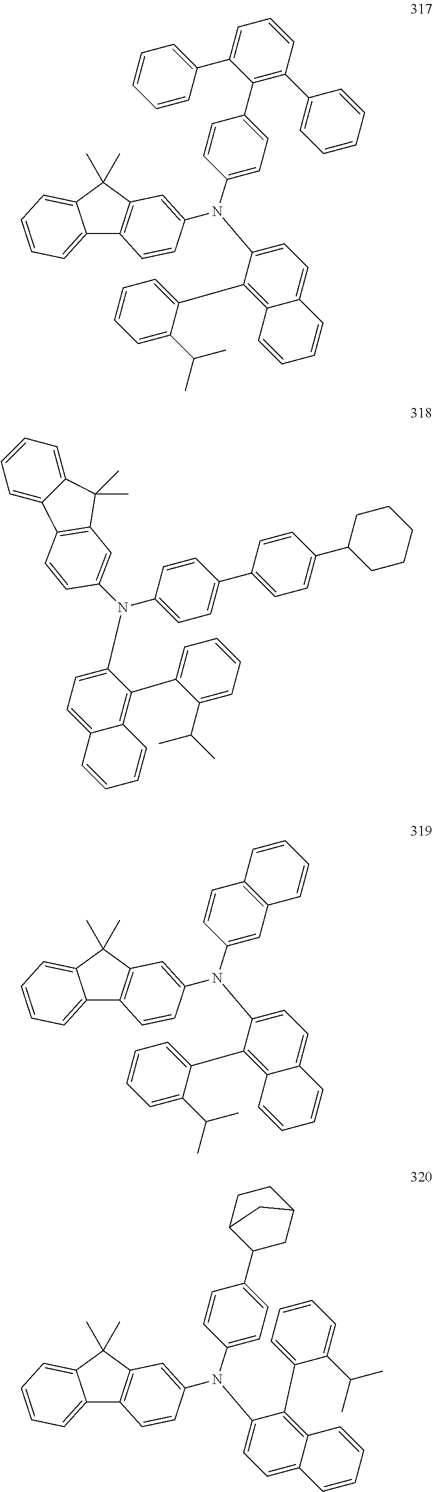 [SMILES: CC(C)c1ccccc1-c1c(N(c2ccc(-c3c(-c4ccccc4)cccc3-c3ccccc3)cc2)c2ccc3c(c2)C(C)(C)c2ccccc2-3)ccc2ccccc12 ; CC(C)c1ccccc1-c1c(N(c2ccc(-c3ccc(C4CCCCC4)cc3)cc2)c2ccc3c(c2)C(C)(C)c2ccccc2-3)ccc2ccccc12 ; CC(C)c1ccccc1-c1c(N(c2ccc3c(c2)C(C)(C)c2ccccc2-3)c2ccc3ccccc3c2)ccc2ccccc12 ; CC(C)c1ccccc1-c1c(N(c2ccc(C3CC4CCC3C4)cc2)c2ccc3c(c2)C(C)(C)c2ccccc2-3)ccc2ccccc12]

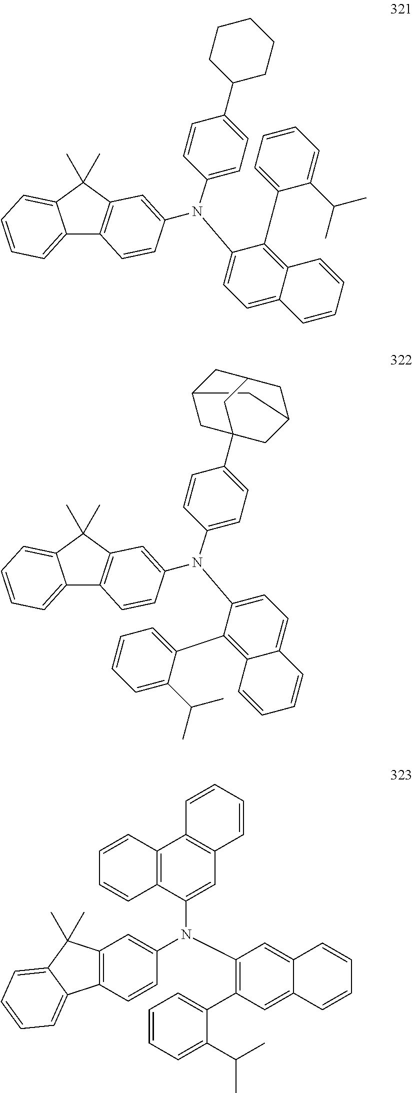 [SMILES: CC(C)c1ccccc1-c1c(N(c2ccc(C3CCCCC3)cc2)c2ccc3c(c2)C(C)(C)c2ccccc2-3)ccc2ccccc12 ; CC(C)c1ccccc1-c1c(N(c2ccc(C34CC5CC(CC(C5)C3)C4)cc2)c2ccc3c(c2)C(C)(C)c2ccccc2-3)ccc2ccccc12 ; CC(C)c1ccccc1-c1cc2ccccc2cc1N(c1ccc2c(c1)C(C)(C)c1ccccc1-2)c1cc2ccccc2c2ccccc12]

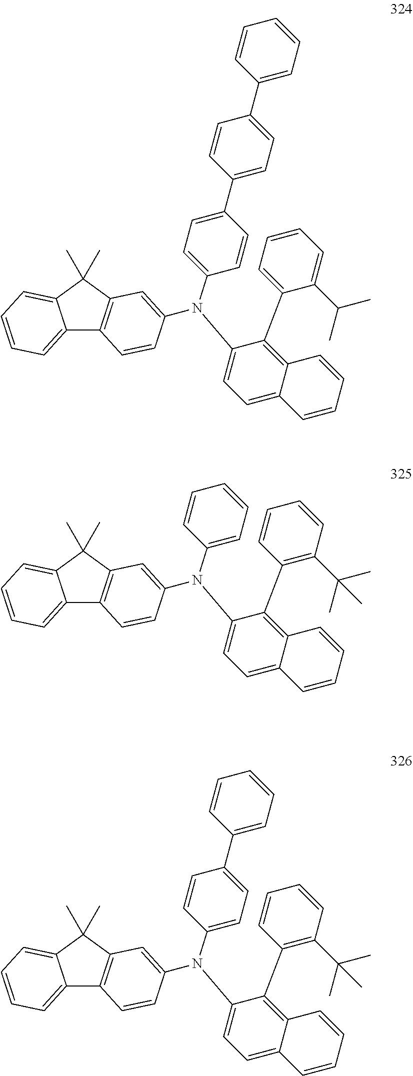 [SMILES: CC(C)c1ccccc1-c1c(N(c2ccc(-c3ccc(-c4ccccc4)cc3)cc2)c2ccc3c(c2)C(C)(C)c2ccccc2-3)ccc2ccccc12 ; CC(C)(C)c1ccccc1-c1c(N(c2ccccc2)c2ccc3c(c2)C(C)(C)c2ccccc2-3)ccc2ccccc12 ; CC(C)(C)c1ccccc1-c1c(N(c2ccc(-c3ccccc3)cc2)c2ccc3c(c2)C(C)(C)c2ccccc2-3)ccc2ccccc12]

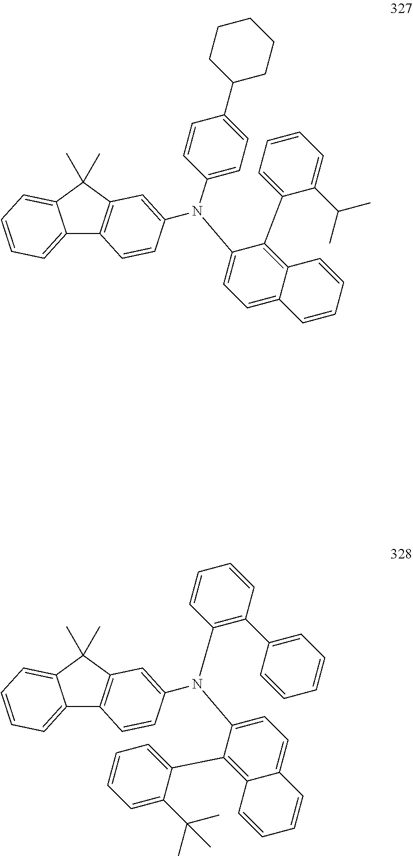 [SMILES: CC(C)c1ccccc1-c1c(N(c2ccc(C3CCCCC3)cc2)c2ccc3c(c2)C(C)(C)c2ccccc2-3)ccc2ccccc12 ; CC(C)(C)c1ccccc1-c1c(N(c2ccc3c(c2)C(C)(C)c2ccccc2-3)c2ccccc2-c2ccccc2)ccc2ccccc12]

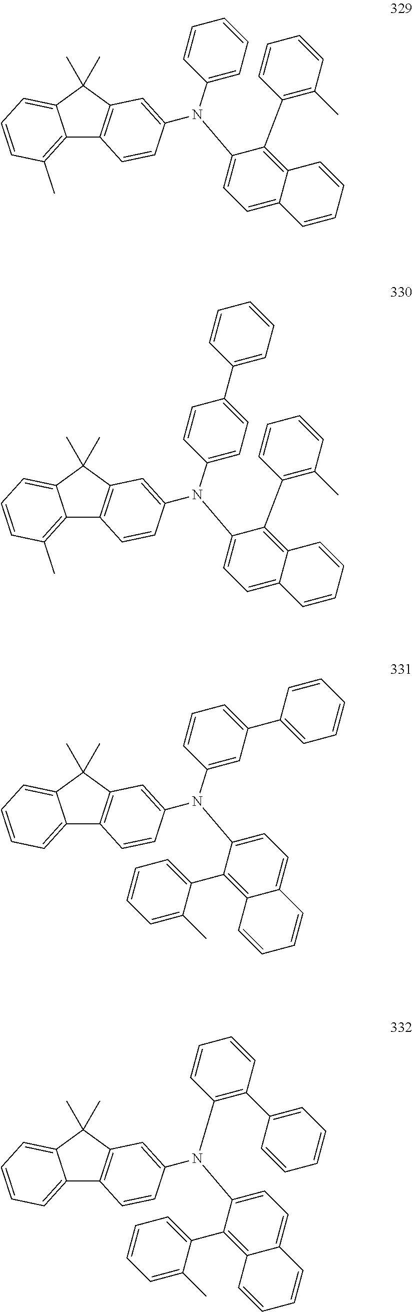 [SMILES: Cc1ccccc1-c1c(N(c2ccccc2)c2ccc3c(c2)C(C)(C)c2cccc(C)c2-3)ccc2ccccc12 ; Cc1ccccc1-c1c(N(c2ccc(-c3ccccc3)cc2)c2ccc3c(c2)C(C)(C)c2cccc(C)c2-3)ccc2ccccc12 ; Cc1ccccc1-c1c(N(c2cccc(-c3ccccc3)c2)c2ccc3c(c2)C(C)(C)c2ccccc2-3)ccc2ccccc12 ; Cc1ccccc1-c1c(N(c2ccc3c(c2)C(C)(C)c2ccccc2-3)c2ccccc2-c2ccccc2)ccc2ccccc12]

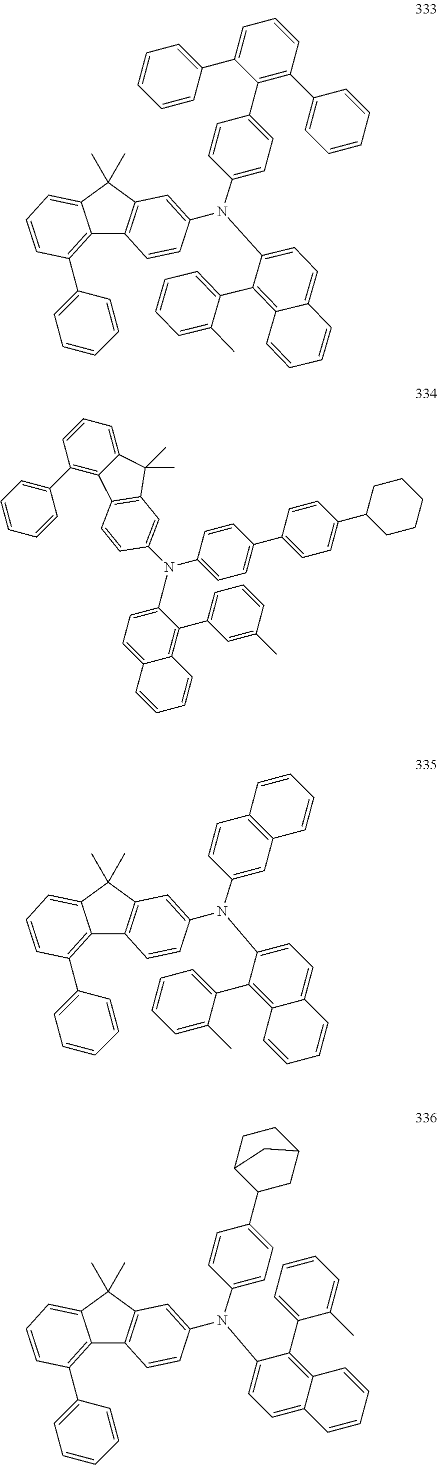 [SMILES: Cc1ccccc1-c1c(N(c2ccc(-c3c(-c4ccccc4)cccc3-c3ccccc3)cc2)c2ccc3c(c2)C(C)(C)c2cccc(-c4ccccc4)c2-3)ccc2ccccc12 ; Cc1cccc(-c2c(N(c3ccc(-c4ccc(C5CCCCC5)cc4)cc3)c3ccc4c(c3)C(C)(C)c3cccc(-c5ccccc5)c3-4)ccc3ccccc23)c1 ; Cc1ccccc1-c1c(N(c2ccc3c(c2)C(C)(C)c2cccc(-c4ccccc4)c2-3)c2ccc3ccccc3c2)ccc2ccccc12 ; Cc1ccccc1-c1c(N(c2ccc(C3CC4CCC3C4)cc2)c2ccc3c(c2)C(C)(C)c2cccc(-c4ccccc4)c2-3)ccc2ccccc12]

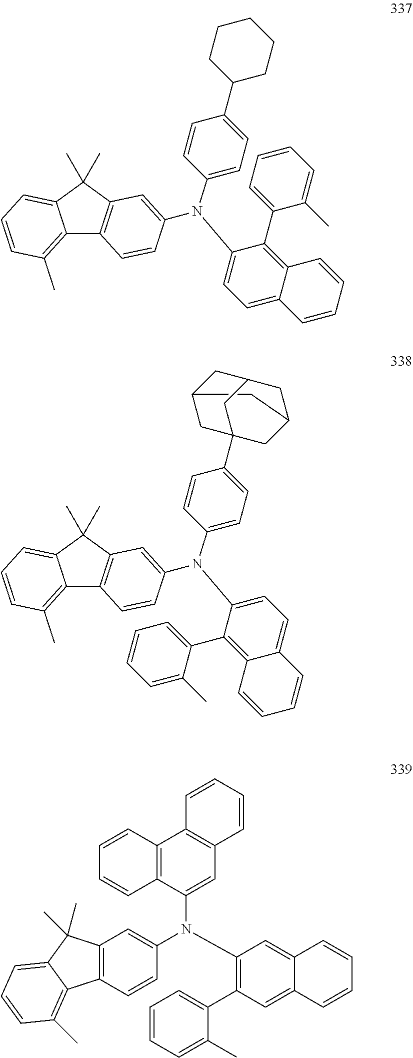 [SMILES: Cc1ccccc1-c1c(N(c2ccc(C3CCCCC3)cc2)c2ccc3c(c2)C(C)(C)c2cccc(C)c2-3)ccc2ccccc12 ; Cc1ccccc1-c1c(N(c2ccc(C34CC5CC(CC(C5)C3)C4)cc2)c2ccc3c(c2)C(C)(C)c2cccc(C)c2-3)ccc2ccccc12 ; Cc1ccccc1-c1cc2ccccc2cc1N(c1ccc2c(c1)C(C)(C)c1cccc(C)c1-2)c1cc2ccccc2c2ccccc12]

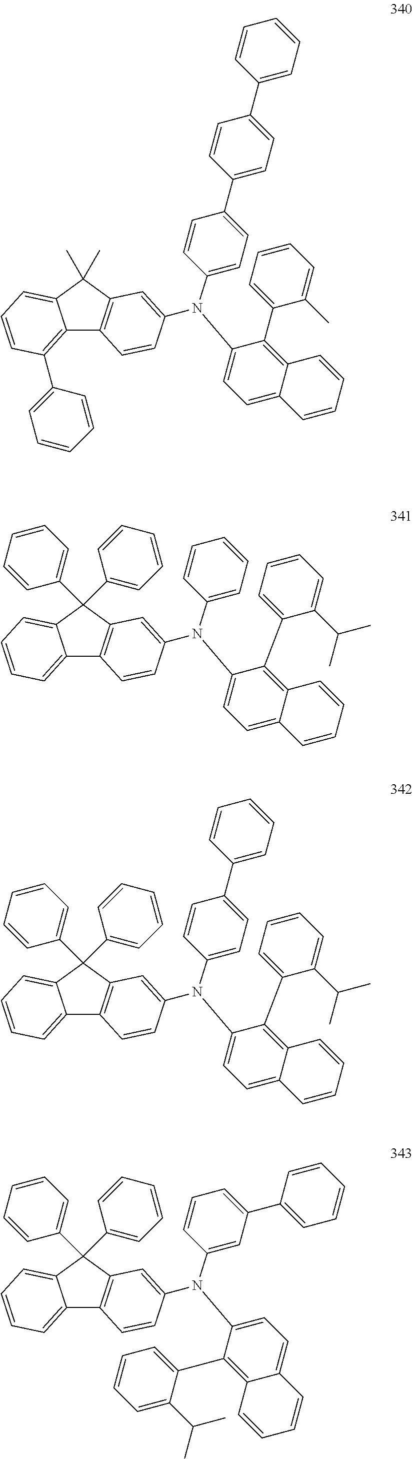 [SMILES: Cc1ccccc1-c1c(N(c2ccc(-c3ccc(-c4ccccc4)cc3)cc2)c2ccc3c(c2)C(C)(C)c2cccc(-c4ccccc4)c2-3)ccc2ccccc12 ; CC(C)c1ccccc1-c1c(N(c2ccccc2)c2ccc3c(c2)C(c2ccccc2)(c2ccccc2)c2ccccc2-3)ccc2ccccc12 ; CC(C)c1ccccc1-c1c(N(c2ccc(-c3ccccc3)cc2)c2ccc3c(c2)C(c2ccccc2)(c2ccccc2)c2ccccc2-3)ccc2ccccc12 ; CC(C)c1ccccc1-c1c(N(c2cccc(-c3ccccc3)c2)c2ccc3c(c2)C(c2ccccc2)(c2ccccc2)c2ccccc2-3)ccc2ccccc12]

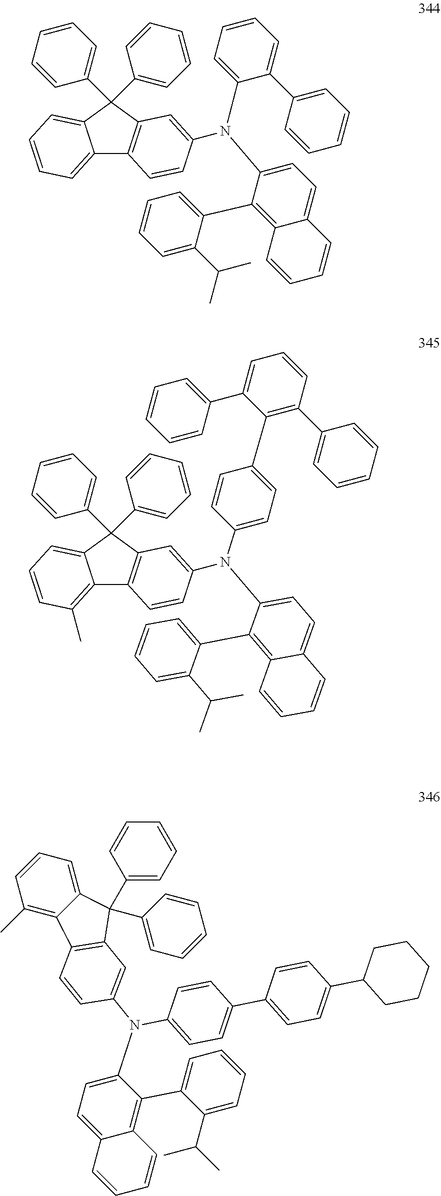 [SMILES: CC(C)c1ccccc1-c1c(N(c2ccc3c(c2)C(c2ccccc2)(c2ccccc2)c2ccccc2-3)c2ccccc2-c2ccccc2)ccc2ccccc12 ; Cc1cccc2c1-c1ccc(N(c3ccc(-c4c(-c5ccccc5)cccc4-c4ccccc4)cc3)c3ccc4ccccc4c3-c3ccccc3C(C)C)cc1C2(c1ccccc1)c1ccccc1 ; Cc1cccc2c1-c1ccc(N(c3ccc(-c4ccc(C5CCCCC5)cc4)cc3)c3ccc4ccccc4c3-c3ccccc3C(C)C)cc1C2(c1ccccc1)c1ccccc1]

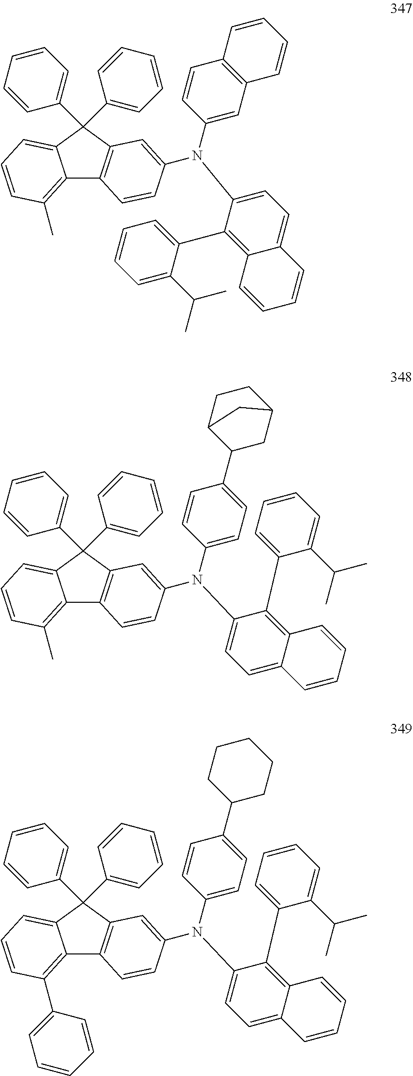 [SMILES: Cc1cccc2c1-c1ccc(N(c3ccc4ccccc4c3)c3ccc4ccccc4c3-c3ccccc3C(C)C)cc1C2(c1ccccc1)c1ccccc1 ; Cc1cccc2c1-c1ccc(N(c3ccc(C4CC5CCC4C5)cc3)c3ccc4ccccc4c3-c3ccccc3C(C)C)cc1C2(c1ccccc1)c1ccccc1 ; CC(C)c1ccccc1-c1c(N(c2ccc(C3CCCCC3)cc2)c2ccc3c(c2)C(c2ccccc2)(c2ccccc2)c2cccc(-c4ccccc4)c2-3)ccc2ccccc12]

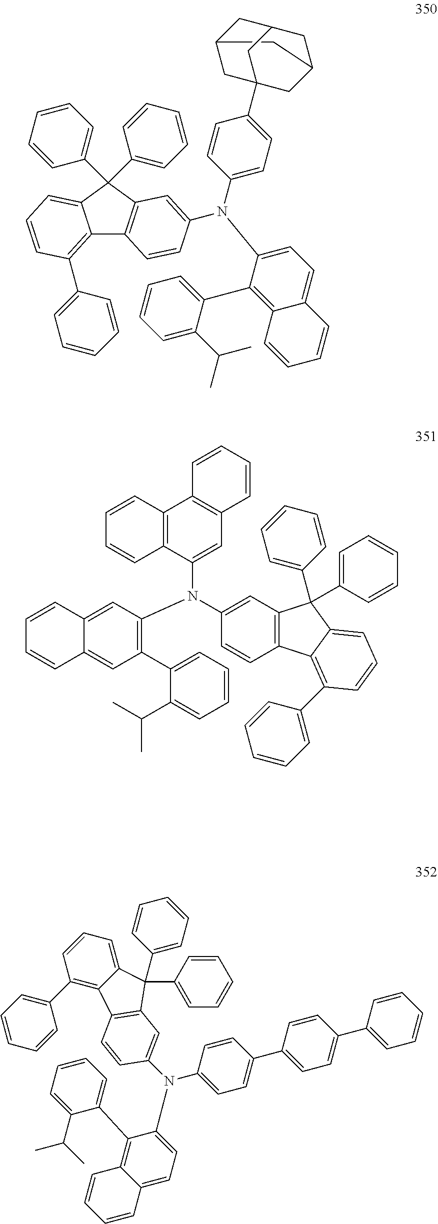 [SMILES: CC(C)c1ccccc1-c1c(N(c2ccc(C34CC5CC(CC(C5)C3)C4)cc2)c2ccc3c(c2)C(c2ccccc2)(c2ccccc2)c2cccc(-c4ccccc4)c2-3)ccc2ccccc12 ; CC(C)c1ccccc1-c1cc2ccccc2cc1N(c1ccc2c(c1)C(c1ccccc1)(c1ccccc1)c1cccc(-c3ccccc3)c1-2)c1cc2ccccc2c2ccccc12 ; CC(C)c1ccccc1-c1c(N(c2ccc(-c3ccc(-c4ccccc4)cc3)cc2)c2ccc3c(c2)C(c2ccccc2)(c2ccccc2)c2cccc(-c4ccccc4)c2-3)ccc2ccccc12]

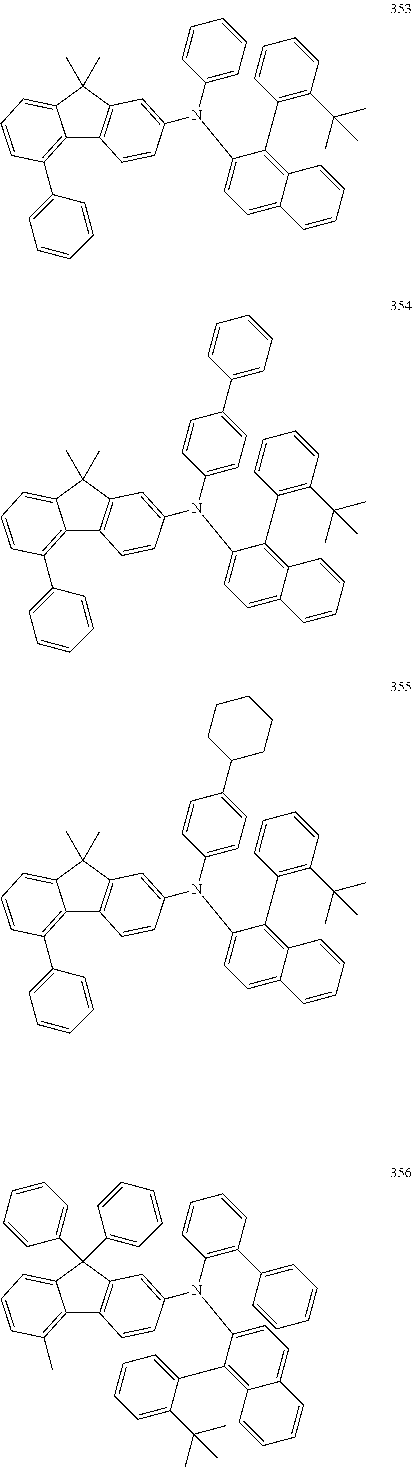 [SMILES: CC(C)(C)c1ccccc1-c1c(N(c2ccccc2)c2ccc3c(c2)C(C)(C)c2cccc(-c4ccccc4)c2-3)ccc2ccccc12 ; CC(C)(C)c1ccccc1-c1c(N(c2ccc(-c3ccccc3)cc2)c2ccc3c(c2)C(C)(C)c2cccc(-c4ccccc4)c2-3)ccc2ccccc12 ; CC(C)(C)c1ccccc1-c1c(N(c2ccc(C3CCCCC3)cc2)c2ccc3c(c2)C(C)(C)c2cccc(-c4ccccc4)c2-3)ccc2ccccc12 ; Cc1cccc2c1-c1ccc(N(c3ccccc3-c3ccccc3)c3ccc4ccccc4c3-c3ccccc3C(C)(C)C)cc1C2(c1ccccc1)c1ccccc1]

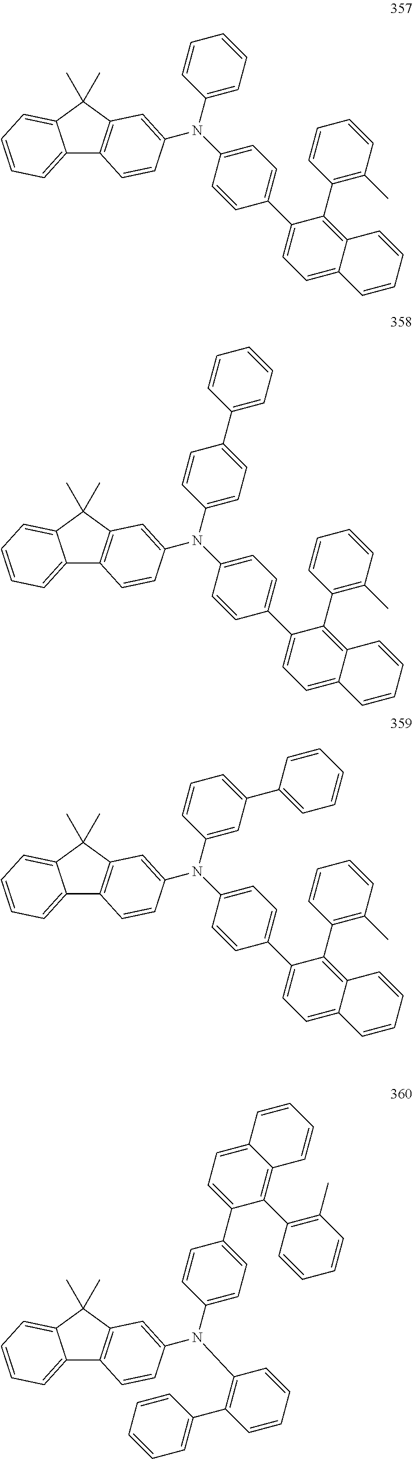 [SMILES: Cc1ccccc1-c1c(-c2ccc(N(c3ccccc3)c3ccc4c(c3)C(C)(C)c3ccccc3-4)cc2)ccc2ccccc12 ; Cc1ccccc1-c1c(-c2ccc(N(c3ccc(-c4ccccc4)cc3)c3ccc4c(c3)C(C)(C)c3ccccc3-4)cc2)ccc2ccccc12 ; Cc1ccccc1-c1c(-c2ccc(N(c3cccc(-c4ccccc4)c3)c3ccc4c(c3)C(C)(C)c3ccccc3-4)cc2)ccc2ccccc12 ; Cc1ccccc1-c1c(-c2ccc(N(c3ccc4c(c3)C(C)(C)c3ccccc3-4)c3ccccc3-c3ccccc3)cc2)ccc2ccccc12]

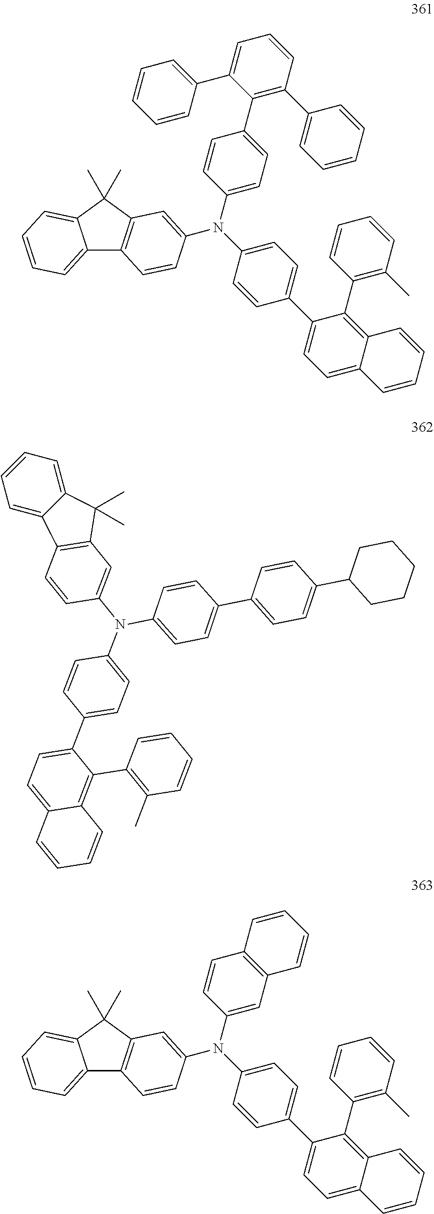 [SMILES: Cc1ccccc1-c1c(-c2ccc(N(c3ccc(-c4c(-c5ccccc5)cccc4-c4ccccc4)cc3)c3ccc4c(c3)C(C)(C)c3ccccc3-4)cc2)ccc2ccccc12 ; Cc1ccccc1-c1c(-c2ccc(N(c3ccc(-c4ccc(C5CCCCC5)cc4)cc3)c3ccc4c(c3)C(C)(C)c3ccccc3-4)cc2)ccc2ccccc12 ; Cc1ccccc1-c1c(-c2ccc(N(c3ccc4c(c3)C(C)(C)c3ccccc3-4)c3ccc4ccccc4c3)cc2)ccc2ccccc12]

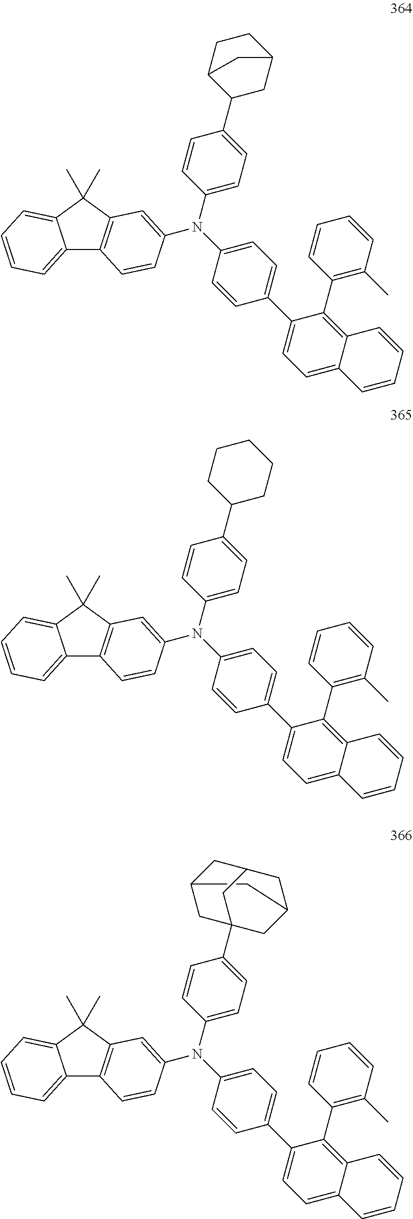 [SMILES: Cc1ccccc1-c1c(-c2ccc(N(c3ccc(C4CC5CCC4C5)cc3)c3ccc4c(c3)C(C)(C)c3ccccc3-4)cc2)ccc2ccccc12 ; Cc1ccccc1-c1c(-c2ccc(N(c3ccc(C4CCCCC4)cc3)c3ccc4c(c3)C(C)(C)c3ccccc3-4)cc2)ccc2ccccc12 ; Cc1ccccc1-c1c(-c2ccc(N(c3ccc(C45CC6CC(CC(C6)C4)C5)cc3)c3ccc4c(c3)C(C)(C)c3ccccc3-4)cc2)ccc2ccccc12]

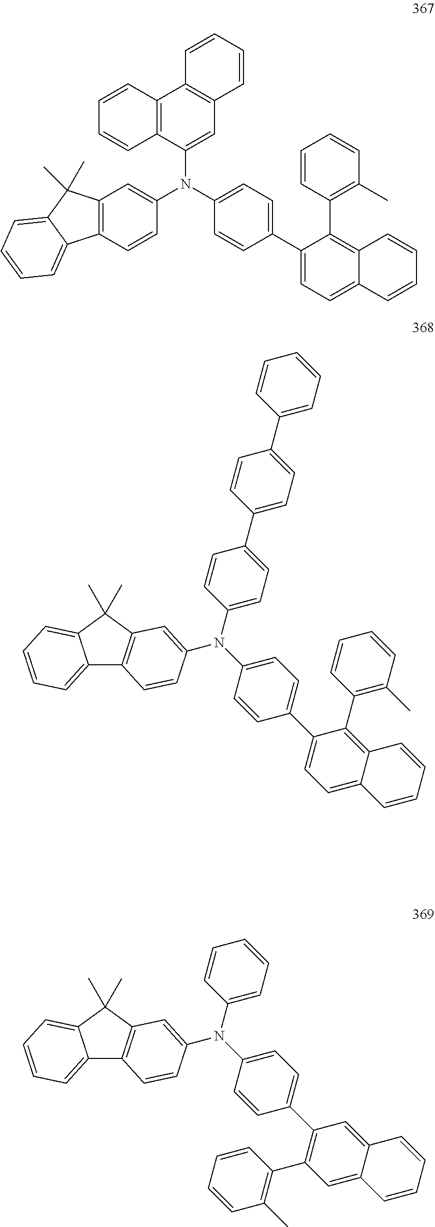 [SMILES: Cc1ccccc1-c1c(-c2ccc(N(c3ccc4c(c3)C(C)(C)c3ccccc3-4)c3cc4ccccc4c4ccccc34)cc2)ccc2ccccc12 ; Cc1ccccc1-c1c(-c2ccc(N(c3ccc(-c4ccc(-c5ccccc5)cc4)cc3)c3ccc4c(c3)C(C)(C)c3ccccc3-4)cc2)ccc2ccccc12 ; Cc1ccccc1-c1cc2ccccc2cc1-c1ccc(N(c2ccccc2)c2ccc3c(c2)C(C)(C)c2ccccc2-3)cc1]

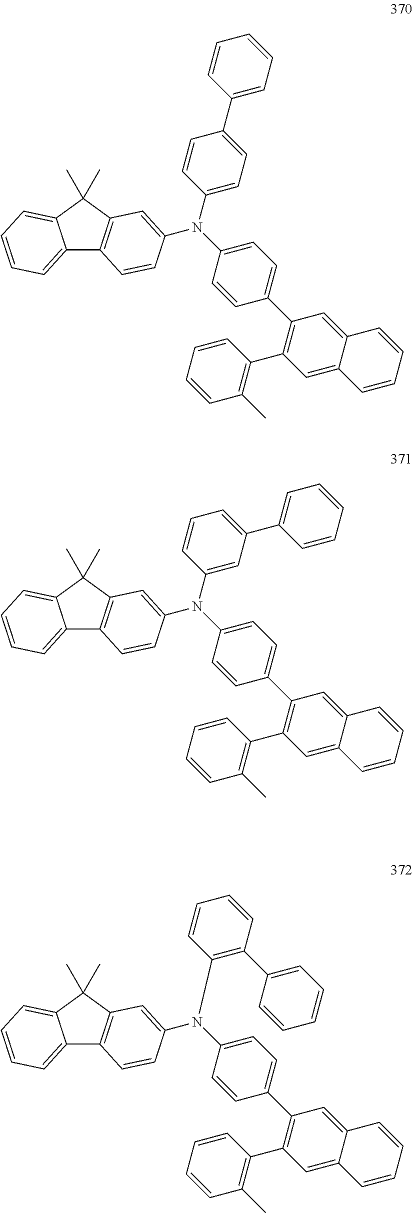 [SMILES: Cc1ccccc1-c1cc2ccccc2cc1-c1ccc(N(c2ccc(-c3ccccc3)cc2)c2ccc3c(c2)C(C)(C)c2ccccc2-3)cc1 ; Cc1ccccc1-c1cc2ccccc2cc1-c1ccc(N(c2cccc(-c3ccccc3)c2)c2ccc3c(c2)C(C)(C)c2ccccc2-3)cc1 ; Cc1ccccc1-c1cc2ccccc2cc1-c1ccc(N(c2ccc3c(c2)C(C)(C)c2ccccc2-3)c2ccccc2-c2ccccc2)cc1]

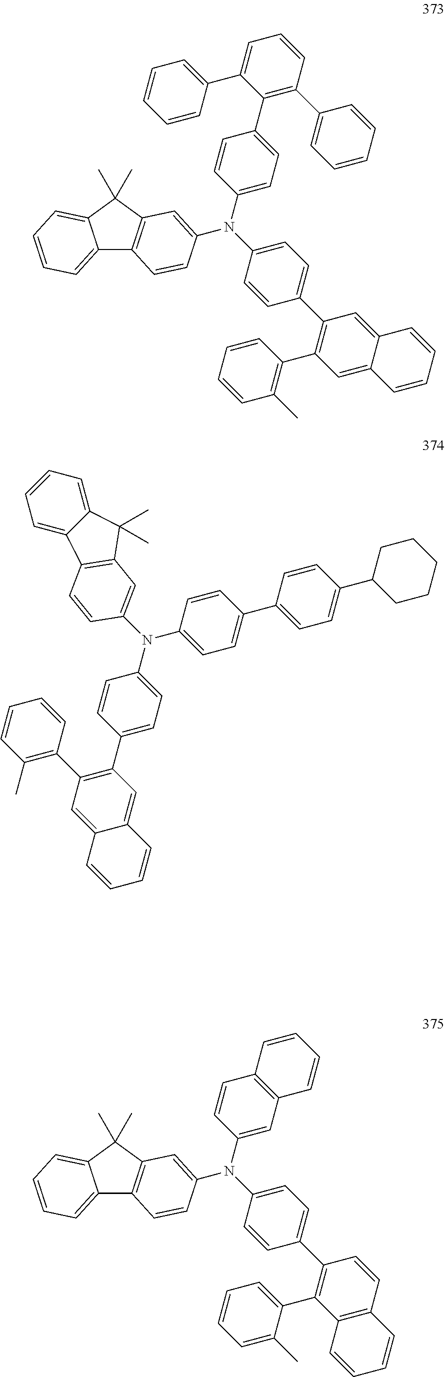 [SMILES: Cc1ccccc1-c1cc2ccccc2cc1-c1ccc(N(c2ccc(-c3c(-c4ccccc4)cccc3-c3ccccc3)cc2)c2ccc3c(c2)C(C)(C)c2ccccc2-3)cc1 ; Cc1ccccc1-c1cc2ccccc2cc1-c1ccc(N(c2ccc(-c3ccc(C4CCCCC4)cc3)cc2)c2ccc3c(c2)C(C)(C)c2ccccc2-3)cc1 ; Cc1ccccc1-c1c(-c2ccc(N(c3ccc4c(c3)C(C)(C)c3ccccc3-4)c3ccc4ccccc4c3)cc2)ccc2ccccc12]

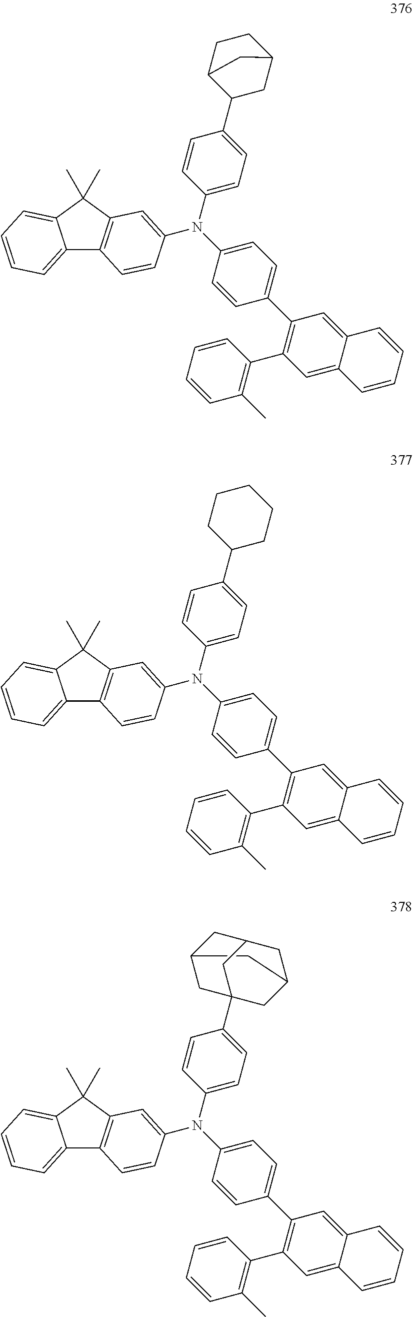 [SMILES: Cc1ccccc1-c1cc2ccccc2cc1-c1ccc(N(c2ccc(C3CC4CCC3C4)cc2)c2ccc3c(c2)C(C)(C)c2ccccc2-3)cc1 ; Cc1ccccc1-c1cc2ccccc2cc1-c1ccc(N(c2ccc(C3CCCCC3)cc2)c2ccc3c(c2)C(C)(C)c2ccccc2-3)cc1 ; Cc1ccccc1-c1cc2ccccc2cc1-c1ccc(N(c2ccc(C34CC5CC(CC(C5)C3)C4)cc2)c2ccc3c(c2)C(C)(C)c2ccccc2-3)cc1]

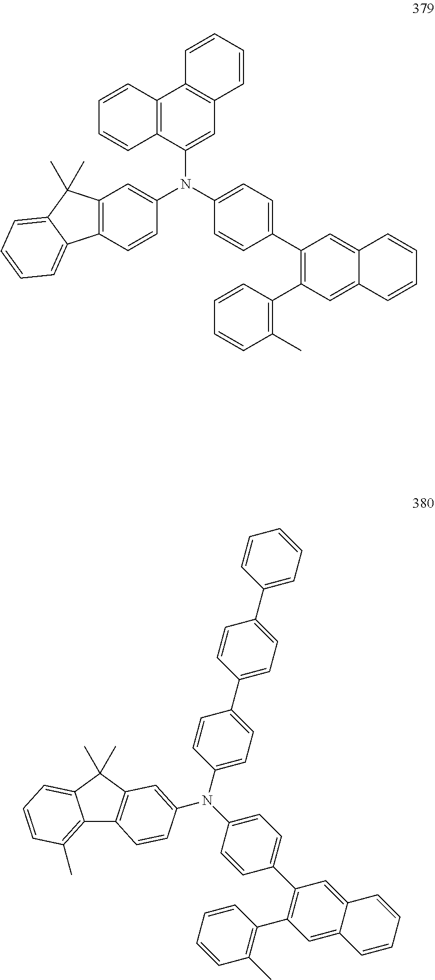 [SMILES: Cc1ccccc1-c1cc2ccccc2cc1-c1ccc(N(c2ccc3c(c2)C(C)(C)c2ccccc2-3)c2cc3ccccc3c3ccccc23)cc1 ; Cc1ccccc1-c1cc2ccccc2cc1-c1ccc(N(c2ccc(-c3ccc(-c4ccccc4)cc3)cc2)c2ccc3c(c2)C(C)(C)c2cccc(C)c2-3)cc1]

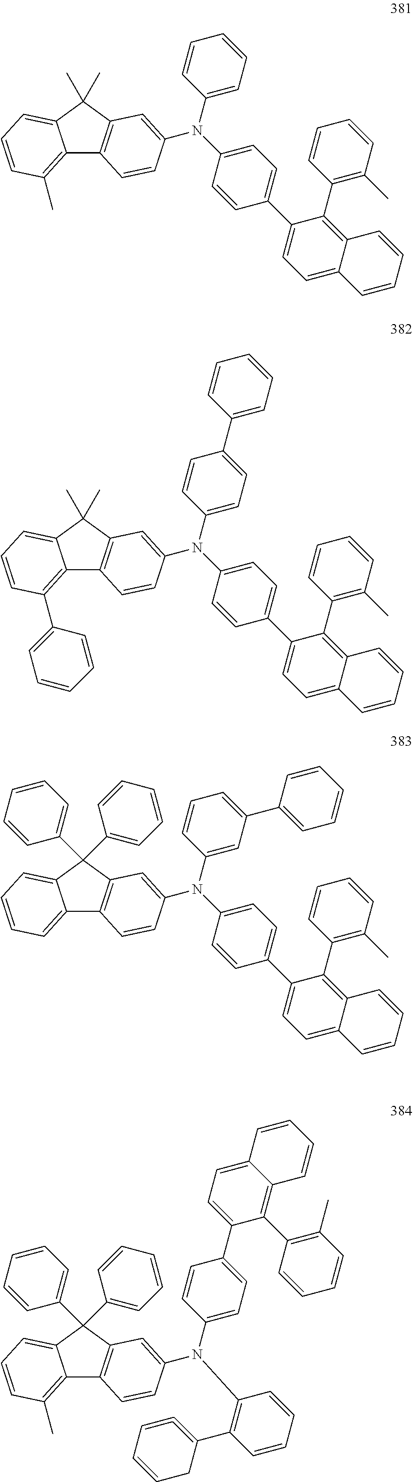 [SMILES: Cc1ccccc1-c1c(-c2ccc(N(c3ccccc3)c3ccc4c(c3)C(C)(C)c3cccc(C)c3-4)cc2)ccc2ccccc12 ; Cc1ccccc1-c1c(-c2ccc(N(c3ccc(-c4ccccc4)cc3)c3ccc4c(c3)C(C)(C)c3cccc(-c5ccccc5)c3-4)cc2)ccc2ccccc12 ; Cc1ccccc1-c1c(-c2ccc(N(c3cccc(-c4ccccc4)c3)c3ccc4c(c3)C(c3ccccc3)(c3ccccc3)c3ccccc3-4)cc2)ccc2ccccc12 ; Cc1ccccc1-c1c(-c2ccc(N(c3ccc4c(c3)C(c3ccccc3)(c3ccccc3)c3cccc(C)c3-4)c3ccccc3C3=C=CC=CC3)cc2)ccc2ccccc12]

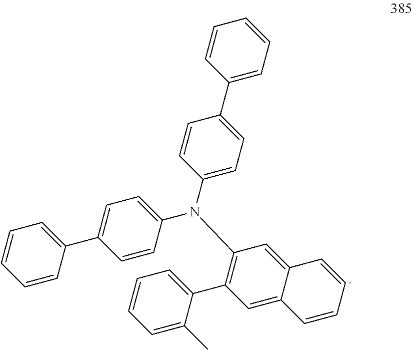 [SMILES: Cc1ccccc1-c1cc2ccccc2cc1N(c1ccc(-c2ccccc2)cc1)c1ccc(-c2ccccc2)cc1]

In some embodiments, the amine-containing compound represented by Formula 1 or 2 may include a naphthalene moiety linked to an amine and a phenyl moiety linked to the naphthalene moiety. In some embodiments, the phenyl moiety may include a substituent R₁ that is ortho to the naphthalene moiety, and R₁ may be deuterium, —F, —Cl, —Br, —I a hydroxyl group, a cyano group, a nitro group, a C₁-C₆₀ alkyl group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂).

In some embodiments, the naphthalene moiety and/or the phenyl moiety may increase the volume and, simultaneously, reduce the symmetry of the amine-containing compound. In some embodiments, crystallization of the amine-containing compound may be suppressed when utilized with, or applied to, the light-emitting device, and the film quality and/or efficiency of the light-emitting device may be enhanced or improved.

In some embodiments, the amine-containing compound may enhance or improve the lifespan of the light-emitting device by including a phenyl and/or naphthalene moiety having a high charge resistance.

In some embodiments, the phenyl moiety with the substituent R₁ ortho to the naphthalene moiety may reduce interactions between individual molecules of the amine-containing compound, and thereby reduce or lower the packing density of the amine-containing compound.

In some embodiments, the amine-containing compound may be characterized by low refractive characteristics.

In some embodiments, the glass transition temperature of the amine-containing compound may be controlled or determined by the type or kind of R₁. The glass transition temperature may be enhanced or improved depending on the type or kind of R₁.

In an embodiment, the highest occupied molecular orbital (HOMO) energy level may change depending on the type or kind of R₁. In an embodiment, a hole injection barrier between the first electrode and the hole transport region of the light-emitting device may vary, (i.e., not be constant). In an embodiment, the barrier may be adjusted to have a suitable energy level between the hole transport region and an emission layer to enhance or improve exciton production efficiency in the emission layer. In an embodiment, when the amine-containing compound is applied to the hole transport region of the light-emitting device, a light-emitting device having high efficiency, low driving voltage, high luminance, and long lifespan may be manufactured or implemented. In an embodiment, when R₁ is an alkyl group, the driving voltage may be statistically enhanced or improved.

Synthesis methods of the amine-containing compound represented by Formula 1 or 2 may be recognizable by one of ordinary skill in the art by referring to the Synthesis Examples and/or Examples described herein.

At least one amine-containing compound represented by Formula 1 or 2 may be utilized in the light-emitting device (for example, an organic light-emitting device) of the present disclosure. Therefore, provided is a light-emitting device including: a first electrode; a second electrode facing the first electrode; an interlayer arranged between the first electrode and the second electrode and including an emission layer; and the amine-containing compound represented by Formula 1 or 2.

In some embodiments, the first electrode of the light-emitting device may be an anode. In some embodiments, the second electrode of the light-emitting device may be a cathode.

In some embodiments, the interlayer may include a hole transport region between the first electrode and the emission layer. In some embodiments, the interlayer may include an electron transport region between the emission layer and the second electrode.

In some embodiments, the hole transport region may include a hole injection layer, a hole transport layer, an emission auxiliary layer, an electron blocking layer, or a combination thereof, and

In some embodiments, the electron transport region may include a hole blocking layer, an electron transport layer, an electron injection layer, an electron control layer, or a combination thereof.

In some embodiments, the amine-containing compound represented by Formula 1 or 2 may be included in the interlayer.

In some embodiments, the amine-containing compound represented by Formula 1 or 2 may be included in the hole transport region.

In some embodiments, the amine-containing compound represented by Formula 1 or 2 may be included in the hole transport layer. In some embodiments, the hole transport layer contacts the emission layer. In some embodiments, the hole transport layer may be in direct contact with the emission layer.

In some embodiments, the emission layer may emit blue light. For example, the emission layer may emit blue light having a maximum emission wavelength of about 400 nm to about 500 nm, about 410 nm to about 490 nm, about 420 nm to about 480 nm, or about 430 nm to about 470 nm.

In some embodiments, the emission layer may include a dopant and/or a host.

In some embodiments, the electron transport region may include a hole blocking layer. In some embodiments, the hole blocking layer may include a phosphine oxide-containing compound, a silicon-containing compound, or a combination thereof. In an embodiment, the hole blocking layer contacts the emission layer. In some embodiments, the hole blocking layer may be in direct contact with the emission layer.

In an embodiment, the light-emitting device may include a capping layer. In an embodiment, a capping layer may located outside (e.g., be external to or on) the first electrode. In an embodiment, a capping layer may located outside (e.g., be external to or on) the second electrode. In an embodiment, the amine-containing compound may be included in the capping layer.

For example, the light-emitting device may include at least one of a first capping layer located outside the first electrode or a second capping layer located outside the second electrode. In an embodiment, at least one of the first capping layer or the second capping layer may include the amine-containing compound. More details for the first capping layer and/or second capping layer are described elsewhere herein.

In an embodiment, the light-emitting device may include:
a first capping layer external to, or located outside, the first electrode and including the amine-containing compound represented by Formula 1 or 2;
a second capping layer external to, or located outside, the second electrode and including the amine-containing compound represented by Formula 1 or 2.

In an embodiment, the light-emitting device may include:
the first capping layer and the second capping layer.

The expression “(an interlayer and/or a capping layer) includes an amine-containing compound” as used herein may include a case in which “(an interlayer and/or a capping layer) includes identical amine-containing compounds represented by Formula 1 or 2” and a case in which “(an interlayer and/or a capping layer) includes two or more different amine-containing compounds represented by Formula 1 or 2.”

In an embodiment, the interlayer and/or capping layer may include Compound 1 only as the amine-containing compound. In this embodiment, Compound 1 may be included in the hole transport layer and/or the emission layer of the light-emitting device. In an embodiment, the interlayer may include, as the amine-containing compound, Compounds 1 and 2. In this regard, Compounds 1 and 2 may be present in substantially the same layer. For example, both (e.g., simultaneously) Compounds 1 and 2 may be present in an emission layer, or may be present in different layers. For example, Compound 1 may be present in an emission layer, and Compound 2 may be present in a hole transport region. Compounds 1 and 2 are described in detail in Synthesis Examples 1 and 2, respectively.

The term “interlayer” as utilized herein refers to a single layer and/or all of a plurality of layers located between the first electrode and the second electrode of the light-emitting device.

In some embodiments, the emission layer may include a compound represented by Formula 6:

M₆₁(L₆₁)ₙ₆₁(L₆₂)ₙ₆₂ Formula 6

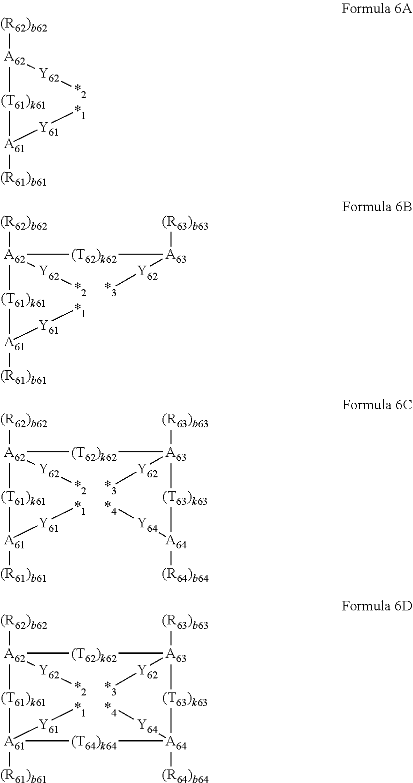

wherein, in Formula 6,
M₆₁ may be selected from a first-row transition metal of the Periodic Table of Elements, a second-row transition metal of the Periodic Table of Elements, and a third-row transition metal of the Periodic Table of Elements,
L₆₁ may be a ligand represented by Formulae 6A to 6D,
L₆₂ may be selected from a monodentate ligand, a bidentate ligand, and a tridentate ligand,
n61 may be selected from 1 and 2,
n62 may be selected from 0, 1, 2, 3, and 4,
A₆₁ to A₆₄ may each independently be selected from a C₅-C₃₀ carbocyclic group and a C₁-C₃₀ heterocyclic group,
T₆₁ to T₆₄ may each independently be selected from a single bond, a double bond, *—S—*′, *—C(═O)—*′, *—S(═O)—*′, *—C(R₆₅)(R₆₆)—*′, *—C(R₆₅)═C(R₆₆)—*′, *—C(R₆₅)=*′, *—Si(R₆₅)(R₆₆)—*′, *—B(R₆₅)—*′, *—N(R₆₅)—*′, and *—P(R₆₅)—*′,
k61 to k64 may each independently be selected from 1, 2, and 3,
Y₆₁ to Y₆₄ may each independently be selected from a single bond (for example, a covalent bond or a coordinate bond), *—O—*′, *—S—*′, *—C(R₆₇)(R₆₈)—*′, *—Si(R₆₇)(R₆₈)—*′, *—B(R₆₇)—*′, *—N(R₆₇)—*′, and *—P(R₆₇)—*′,
*₁, *₂, *₃, and *₄ may be a binding site to M₆₁ (i.e., a point of attachment),
R₆₁ to R₆₈ may each independently be selected from (i.e., selected from among) hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —C(Q₁)(Q₂)(Q₃), —Si(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), and —P(═S)(Q₁)(Q₂),
R₆₁ to R₆₈ may optionally be linked to each other to form a substituted or unsubstituted C₅-C₆₀ carbocyclic group or a substituted or unsubstituted C₁-C₆₀ heterocyclic group, and
b61 to b64 may each independently be selected from an integer from 0 to 10.

For example, M₆₁ in Formula 6 may be selected from platinum (Pt), palladium (Pd), copper (Cu), silver (Ag), gold (Au), rhodium (Rh), iridium (Ir), ruthenium (Ru), osmium (Os), titanium (Ti), zirconium (Zr), hafnium (Hf), europium (Eu), terbium (Tb), and thulium (Tm).

In some embodiments, M₆₁ in Formula 6 may be selected from Pt and Ir.

For example, A₆₁ to A₆₄ in Formulae 6A to 6D may each independently be i) a first ring, ii) a second ring, iii) a condensed ring in which two or more first rings are condensed with each other, iv) a condensed ring in which two or more second rings are condensed with each other, or v) a condensed ring in which one or more first rings and one or more second rings are condensed with each other. In some embodiments, the first ring may be selected from a cyclopentane group, a cyclopentene group, a cyclopentadiene group, a furan group, a thiophene group, a pyrrole group, a borole group, a phosphole group, a silole group, a germole group, a selenophene group, an oxazole group, a dihydroxazole group, an isoxazole group, a dihydroisoxazole group, an oxadiazole group, a dihydroxaddiazole group, an isoxadiazole group, a dihydroisoxadiazole group, an oxatriazole group, a dihydroxatriazole group, an isoxatriazole group, a dihydroisoxatriazole group, a thiazole group, a dihydrothiazole group, an isothiazole group, a dihydroisothiazole group, a thiadiazole group, a dihydrothiadiazole group, an isothiadiazole group, a dihydroisothiadiazole group, a thiatriazole group, a dihydrothiatriazole group, an isothiatriazole group, a dihydroisothiatriazole group, a pyrazole group, a dihydropyrazole group, an imidazole group, a dihydroimidazole group, a triazole group, a dihydrotriazole group, a tetrazole group, a dihydrotetrazole group, an azasilole group, a diazasilole group, and a triazasilole group. In some embodiments, the second ring may be selected from a cyclohexane group, a cyclohexene group, a cyclohexadiene group, an admantane group, a norbornane group, a norbornene group, a benzene group, a pyridine group, a dihydropyridine group, a tetrahydropyridine group, a pyrimidine group, a dihydropyrimidine group, a tetrahydropyrimidine group, a pyrazine group, a dihydropyrazine group, a tetrahydropyrazine group, a pyridazine group, a dihydropyridazine group, a tetrahydropyridazine group, and a triazine group.

In some embodiments, A₆₁ to A₆₄ in Formulae 6A to 6D may each independently be selected from a benzene group, a naphthalene group, an anthracene group, a phenanthrene group, a triphenylene group, a pyrene group, a chrysene group, a furan group, a thiophene group, a silole group, an indene group, a fluorene group, a benzofuran group, a dibenzofuran group, a benzothiophene group, a dibenzothiophene group, a benzosilole group, a dibenzosilole group, an indole group, a carbazole group, an indenopyridine group, an indolopyridine group, a benzofuropyridine group, a benzothienopyridine group, a benzosilolopyridine group, an indenopyrimidine group, an indolopyrimidine group, a benzofuropyrimidine group, a benzothienopyrimidine group, a benzosilolopyrimidine group, a pyridine group, a pyrimidine group, a pyrazine group, a pyridazine group, a triazine group, a quinoline group, an isoquinoline group, a quinoxaline group, a quinazoline group, a cinnoline group, a phthalazine group, a phenanthroline group, a pyrrole group, a pyrazole group, an imidazole group, a dihydroimidazole group, a triazole group, a dihydrotriazole group, an oxazole group, a dihydroxazole group, an isoxazole group, a thiazole group, a dihydrothiazole group, an isothiazole group, an oxadiazole group, a dihydroxaddiazole group, a thiadiazole group, a dihydrothiadiazole group, a benzopyrazole group, a benzimidazole group, a dihydrobenzimidazole group, an imidazopyridine group, an imidazopyrimidine group, an imidazopyrazine group, a benzoxazole group, a dihydrobenzoxazole group, a benzothiazole group, a dihydrobenzothiazole group, a benzoxadiazole group, a dihydrobenzoxadiazole group, a benzothiadiazole group, and a dihydrobenzothiadiazole group.

For example, T₆₁ to T₆₄ in Formulae 6A to 6D may each independently be selected from a single bond, a double bond, *—O—*′, *—S—*′, *—C(R₆₅)(R₆₆)—*′, and *—N(R₆₅)—*′.

For example, Y₆₁ to Y₆₄ in Formula 6A to 6D may each independently be selected from a single bond, *—O—*′, and *—S—*′.

For example, R₆₁ to R₆₈ in Formulae 6A to 6D may each independently be selected from (i.e., at least one selected from the group consisting of):
hydrogen, deuterium, —F, —Cl, —Br, —I, a cyano group, a C₁-C₂₀ alkyl group, and a C₁-C₂₀ alkoxy group;
a phenyl group, a biphenyl group, a terphenyl group, a naphthyl group, a phenanthrenyl group, a triphenylenyl group, a chrysenyl group, a fluoranthenyl group, a fluorenyl group, a carbazolyl group, a dibenzofuranyl group, a dibenzothiophenyl group, a benzofluorenyl group, a benzocarbazolyl group, a benzonaphthofuranyl group, a benzonaphthothiophenyl group, a dibenzofluorenyl group, a dibenzocarbazolyl group, a dinaphthofuranyl group, a dinaphthothiophenyl group, a pyridinyl group, a pyrazinyl group, a pyridazinyl group, a pyrimidinyl group, a triazinyl group, a quinolinyl group, an isoquinolinyl group, a quinolinyl group, an isoquinolinyl group, a naphthyridinyl group, a quinoxalinyl group, a quinazolinyl group, an azafluorenyl group, an azacarbazolyl group, an azadibenzofuranyl group, an azadibenzothiophenyl group, a diazafluorenyl group, a diazacarbazolyl group, a diazadibenzofuranyl group, and a diazadibenzothiophenyl group;
a phenyl group, a biphenyl group, a terphenyl group, a naphthyl group, a phenanthrenyl group, a triphenylenyl group, a chrysenyl group, a fluoranthenyl group, a fluorenyl group, a carbazolyl group, a dibenzofuranyl group, a dibenzothiophenyl group, a benzofluorenyl group, a benzocarbazolyl group, a benzonaphthofuranyl group, a benzonaphthothiophenyl group, a dibenzofluorenyl group, a dibenzocarbazolyl group, a dinaphthofuranyl group, a dinaphthothiophenyl group, a pyridinyl group, a pyrazinyl group, a pyridazinyl group, a pyrimidinyl group, a triazinyl group, a quinolinyl group, an isoquinolinyl group, a quinolinyl group, an isoquinolinyl group, a naphthyridinyl group, a quinoxalinyl group, a quinazolinyl group, an azafluorenyl group, an azacarbazolyl group, an azadibenzofuranyl group, an azadibenzothiophenyl group, a diazafluorenyl group, a diazacarbazolyl group, a diazadibenzofuranyl group, and a diazadibenzothiophenyl group, each substituted with at least one selected from deuterium, —F, —Cl, —Br, —I, a cyano group, a C₁-C₂₀ alkyl group, a C₁-C₂₀ alkoxy group, a phenyl group, a biphenyl group, a terphenyl group, a naphthyl group, a phenanthrenyl group, a triphenylenyl group, a chrysenyl group, a fluoranthenyl group, a fluorenyl group, a carbazolyl group, a dibenzofuranyl group, a dibenzothiophenyl group, a benzofluorenyl group, a benzocarbazolyl group, a benzonaphthofuranyl group, a benzonaphthothiophenyl group, a dibenzofluorenyl group, a dibenzocarbazolyl group, a dinaphthofuranyl group, a dinaphthothiophenyl group, a pyridinyl group, a pyrazinyl group, a pyridazinyl group, a pyrimidinyl group, a triazinyl group, a quinolinyl group, an isoquinolinyl group, a quinolinyl group, an isoquinolinyl group, a naphthyridinyl group, a quinoxalinyl group, a quinazolinyl group, an azafluorenyl group, an azacarbazolyl group, an azadibenzofuranyl group, an azadibenzothiophenyl group, a diazafluorenyl group, a diazacarbazolyl group, a diazadibenzofuranyl group, and a diazadibenzothiophenyl group; and
—B(Q₆₁)(Q₆₂) and —N(Q₆₁)(Q₆₂),
wherein Q₆₁ and Q₆₂ may each independently be selected from:
hydrogen, deuterium and a C₁-C₂₀ alkyl group;
a phenyl group, a biphenyl group, a terphenyl group, a naphthyl group, a phenanthrenyl group, a triphenylenyl group, a chrysenyl group, a fluoranthenyl group, a fluorenyl group, a carbazolyl group, a dibenzofuranyl group, a dibenzothiophenyl group, a benzofluorenyl group, a benzocarbazolyl group, a benzonaphthofuranyl group, a benzonaphthothiophenyl group, a dibenzofluorenyl group, a dibenzocarbazolyl group, a dinaphthofuranyl group, a dinaphthothiophenyl group, a pyridinyl group, a pyrazinyl group, a pyridazinyl group, a pyrimidinyl group, a triazinyl group, a quinolinyl group, an isoquinolinyl group, a quinolinyl group, an isoquinolinyl group, a naphthyridinyl group, a quinoxalinyl group, a quinazolinyl group, an azafluorenyl group, an azacarbazolyl group, an azadibenzofuranyl group, an azadibenzothiophenyl group, a diazafluorenyl group, a diazacarbazolyl group, a diazadibenzofuranyl group, and a diazadibenzothiophenyl group; and
a phenyl group, a biphenyl group, a terphenyl group, a naphthyl group, a phenanthrenyl group, a triphenylenyl group, a chrysenyl group, a fluoranthenyl group, a fluorenyl group, a carbazolyl group, a dibenzofuranyl group, a dibenzothiophenyl group, a benzofluorenyl group, a benzocarbazolyl group, a benzonaphthofuranyl group, a benzonaphthothiophenyl group, a dibenzofluorenyl group, a dibenzocarbazolyl group, a dinaphthofuranyl group, a dinaphthothiophenyl group, a pyridinyl group, a pyrazinyl group, a pyridazinyl group, a pyrimidinyl group, a triazinyl group, a quinolinyl group, an isoquinolinyl group, a quinolinyl group, an isoquinolinyl group, a naphthyridinyl group, a quinoxalinyl group, a quinazolinyl group, an azafluorenyl group, an azacarbazolyl group, an azadibenzofuranyl group, an azadibenzothiophenyl group, a diazafluorenyl group, a diazacarbazolyl group, a diazadibenzofuranyl group, and a diazadibenzothiophenyl group, each substituted with at least one selected from deuterium, a C₁-C₂₀ alkyl group, a phenyl group, a biphenyl group, a terphenyl group, a naphthyl group, a phenanthrenyl group, a triphenylenyl group, a chrysenyl group, a fluoranthenyl group, a fluorenyl group, a carbazolyl group, a dibenzofuranyl group, a dibenzothiophenyl group, a benzofluorenyl group, a benzocarbazolyl group, a benzonaphthofuranyl group, a benzonaphthothiophenyl group, a dibenzofluorenyl group, a dibenzocarbazolyl group, a dinaphthofuranyl group, a dinaphthothiophenyl group, a pyridinyl group, a pyrazinyl group, a pyridazinyl group, a pyrimidinyl group, a triazinyl group, a quinolinyl group, an isoquinolinyl group, a quinolinyl group, an isoquinolinyl group, a naphthyridinyl group, a quinoxalinyl group, a quinazolinyl group, an azafluorenyl group, an azacarbazolyl group, an azadibenzofuranyl group, an azadibenzothiophenyl group, a diazafluorenyl group, a diazacarbazolyl group, a diazadibenzofuranyl group, and a diazadibenzothiophenyl group.

In some embodiments, R₆₁ to R₆₈ in Formulae 6A to 6D may each independently be selected from: hydrogen, deuterium, —F, —Cl, —Br, —I, a cyano group, a methyl group, an ethyl group, an n-propyl group, an isopropyl group, an n-butyl group, an isobutyl group, a sec-butyl group, a tert-butyl group, a methoxy group, an ethoxy group, a propoxy group, and a butoxy group;
a phenyl group, a biphenyl group, a terphenyl group, a naphthyl group, a phenanthrenyl group, a triphenylenyl group, a chrysenyl group, a fluoranthenyl group, a fluorenyl group, a carbazolyl group, a dibenzofuranyl group, and a dibenzothiophenyl group;
a phenyl group, a biphenyl group, a terphenyl group, a naphthyl group, a phenanthrenyl group, a triphenylenyl group, a chrysenyl group, a fluoranthenyl group, a fluorenyl group, a carbazolyl group, a dibenzofuranyl group, and a dibenzothiophenyl group, each substituted with at least one selected from deuterium, —F, —Cl, —Br, —I, a cyano group, a methyl group, an ethyl group, an n-propyl group, an isopropyl group, an n-butyl group, an isobutyl group, a sec-butyl group, a tert-butyl group, a methoxy group, an ethoxy group, a propoxy group, a butoxy group, a phenyl group, a biphenyl group, a terphenyl group, a naphthyl group, a phenanthrenyl group, a triphenylenyl group, a chrysenyl group, a fluoranthenyl group, a fluorenyl group, a carbazolyl group, a dibenzofuranyl group, and a dibenzothiophenyl group; and
—B(Q₆₁)(Q₆₂) and —N(Q₆₁)(Q₆₂),
wherein Q₆₁ and Q₆₂ may each independently be selected from:
hydrogen, deuterium, a methyl group, an ethyl group, an n-propyl group, an isopropyl group, an n-butyl group, an isobutyl group, a sec-butyl group, and a tert-butyl group;
a phenyl group, a biphenyl group, a terphenyl group, a naphthyl group, a phenanthrenyl group, a triphenylenyl group, a chrysenyl group, a fluoranthenyl group, a fluorenyl group, a carbazolyl group, a dibenzofuranyl group, and a dibenzothiophenyl group; and
a phenyl group, a biphenyl group, a terphenyl group, a naphthyl group, a phenanthrenyl group, a triphenylenyl group, a chrysenyl group, a fluoranthenyl group, a fluorenyl group, a carbazolyl group, a dibenzofuranyl group, and a dibenzothiophenyl group, each substituted with at least one selected from deuterium, a methyl group, an ethyl group, an n-propyl group, an isopropyl group, an n-butyl group, an isobutyl group, a sec-butyl group, a tert-butyl group, a phenyl group, a biphenyl group, a terphenyl group, a naphthyl group, a phenanthrenyl group, a triphenylenyl group, a chrysenyl group, a fluoranthenyl group, a fluorenyl group, a carbazolyl group, a dibenzofuranyl group, and a dibenzothiophenyl group.

In an embodiment, the compound represented by Formula 6 may be a carbene complex.

In some embodiments, L₆₁ in the compound represented by Formula 6 may be a ligand represented by Formula 6C.

In some embodiments, the compound represented by Formula 6 may be a carbene complex, and L₆₁ in the compound represented by Formula 6 may be a ligand represented by 6C.

The term “carbene complex” as utilized herein refers to a compound including a metal and a divalent ligand that is coordinated to the metal, wherein at least one bond between the metal and the divalent ligand is between the metal and a carbon atom(s) of the divalent ligand. For example, the palladium (Pd) carbene complex utilized in the synthesis of Intermediate C4, as described in more detail later, includes a bond between Pd and an imidazole ring.

In some embodiments, a compound represented by Formula 6 may be represented by one of Formulae 6-1 to 6-2:

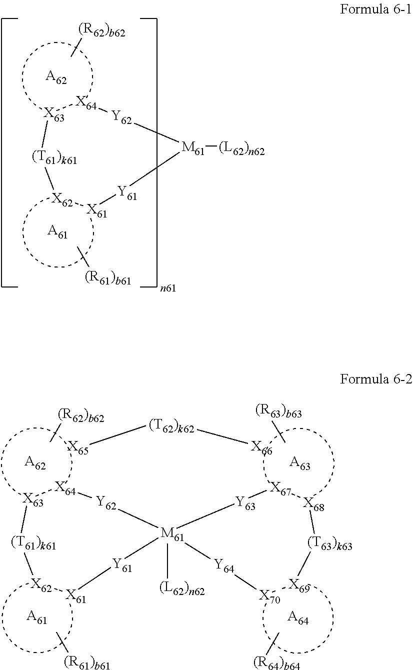

wherein, in Formulae 6-1 and 6-2,
X₆₁ to X₇₀ may each independently be selected from N and C, and
the remaining components may each be understood by referring to the corresponding descriptions thereof provided herein.

In Formulae 6-1 and 6-2, A₆₁ to A₆₄ may each be a ring and may be understood by referring to the descriptions of A₆₁ to A₆₄ in Formula 6.

In an embodiment, in Formula 6-2, X₆₁ may be C, and Y₆₁ may be a coordinate bond. For example, X₆₁ in Formula 6-2 may be a carbon of a carbene.

In some embodiments, X₇₀ of Formula 6-2 may be C, and Y₆₄ may be a coordinate bond. For example, X₇₀ in Formula 6-2 may be a carbon of a carbene.

In some embodiments, a compound represented by Formula 6 may be a carbene complex, and may be a compound represented by Formula 6-2. For example, M₆₁ in Formula 6-2 may be platinum.

In some embodiments, the emission layer may further include at least one of an auxiliary dopant and a sensitizer.

In some embodiments, the auxiliary dopant and the sensitizer may each independently be an organometallic compound including platinum and a tetradentate ligand linked to the platinum, and the tetradentate ligand may include a carbene moiety chemically linked to the platinum. For example, the auxiliary dopant and/or the sensitizer may include the compound represented by Formula 6.

In an embodiment, the sensitizer may include the compound represented by Formula 6.

In an embodiment, the emission layer may further include a first host and a second host, wherein the first host may be an electron transport host, and the second host may be a hole transport host.

In an embodiment, the first host may include at least one azine moiety, and the second host may include at least one carbazole moiety.

In an embodiment, the first host may include a compound represented by Formula 5:

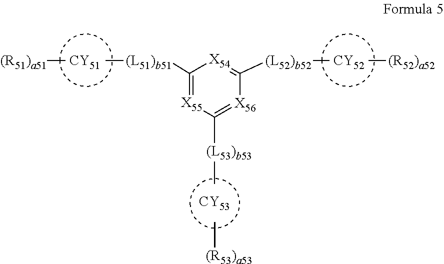

wherein, in Formula 5,
X₅₄ to X₅₆ may each independently be C(R₅₀), CH, or N, and at least one of X₅₄ to X₅₆ may be N,
ring CY₅₁ to ring CY₅₃ may each independently be a C₅-C₆₀ carbocyclic group or a C₁-C₆₀ heterocyclic group,
L₅₁ to L₅₃ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
b51 to b53 may each independently be an integer from 0 to 3,
*-(L₅₁)_{b51}-*′ may be a single bond when b51 is 0,
*-(L₅₂)_{b52}-*′ may be a single bond when b52 is 0,
*-(L₅₃)_{ab3}-*′ may be a single bond when b53 is 0,
R₅₀ to R₅₃ may each be as described for R₁, and
a51 to a53 may each independently be an integer from 0 to 10.

In an embodiment, in Formula 5, ring CY₅₁ to ring CY₅₃ may each independently include i) a first ring, ii) a second ring, iii) a condensed ring in which two or more first rings are condensed with each other, iv) a condensed ring in which two or more second rings are condensed with each other, or v) a condensed ring in which at least one first ring and at least one second ring are condensed with each other,
the first ring may include a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group, and
the second ring may include a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, or an azadibenzofuran group.

In an embodiment, rings CY₅₁ to CY₅₃ in Formula 5 may each independently be a benzene group, a naphthalene group, an anthracene group, a phenanthrene group, a triphenylene group, a pyrene group, a chrysene group, a cyclopentadiene group, a 1,2,3,4-tetrahydronaphthalene group, a thiophene group, a furan group, an indole group, a benzoborole group, a benzophosphole group, an indene group, a benzosilole group, a benzogermole group, a benzothiophene group, a benzoselenophene group, a benzofuran group, a carbazole group, a dibenzoborole group, a dibenzophosphole group, a fluorene group, a dibenzosilole group, a dibenzogermole group, a dibenzothiophene group, a dibenzoselenophene group, a dibenzofuran group, a dibenzothiophene 5-oxide group, a 9H-fluorene-9-one group, a dibenzothiophene 5,5-dioxide group, an azaindole group, an azabenzoborole group, an azabenzophosphole group, an azaindene group, an azabenzosilole group, an azabenzogermole group, an azabenzothiophene group, an azabenzoselenophene group, an azabenzofuran group, an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene 5-oxide group, an aza-9H-fluorene-9-one group, an azadibenzothiophene 5,5-dioxide group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isoxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a 5,6,7,8-tetrahydroisoquinoline group, or a 5,6,7,8-tetrahydroquinoline group.

In an embodiment, L₅₁ to L₅₃ in Formula 5 may each independently be a benzene group, a naphthalene group, an anthracene group, a phenanthrene group, a triphenylene group, a pyrene group, a chrysene group, cyclopentadiene group, a furan group, a thiophene group, a silole group, an indene group, a fluorene group, an indole group, a carbazole group, a benzofuran group, a dibenzofuran group, a benzothiophene group, a dibenzothiophene group, a benzosilole group, a dibenzosilole group, an azafluorene group, an azacarbazole group, an azadibenzofuran group, an azadibenzothiophene group, an azadibenzosilole group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isoxazole group, a thiazole group, an isothiazole group, an oxadiazole group, a thiadiazole group, a benzopyrazole group, a benzimidazole group, a benzoxazole group, a benzothiazole group, a benzoxadiazole group, or a benzothiadiazole group.

In an embodiment, the first host may include at least one of compounds represented by Formulae ETH1 and ETH32:

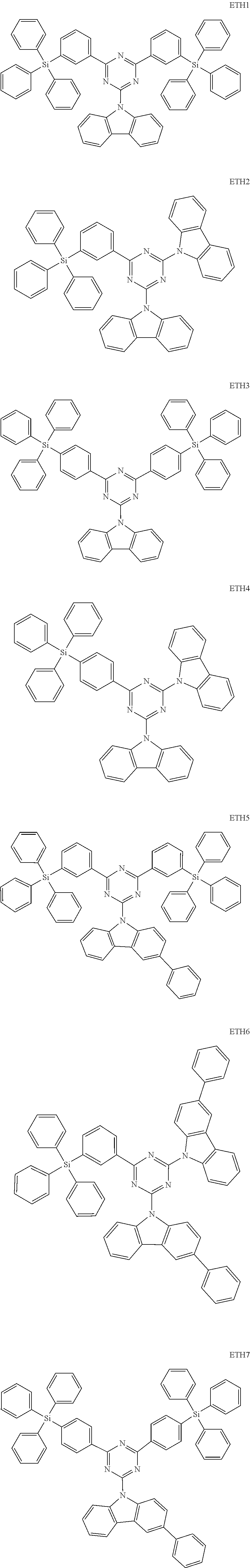

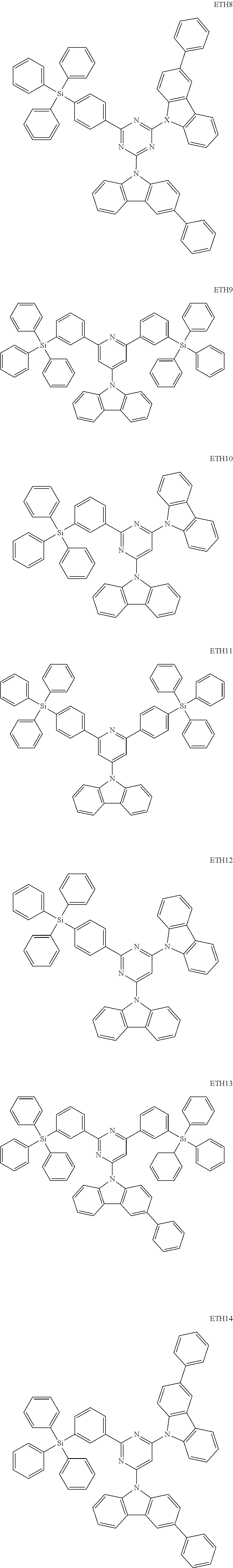

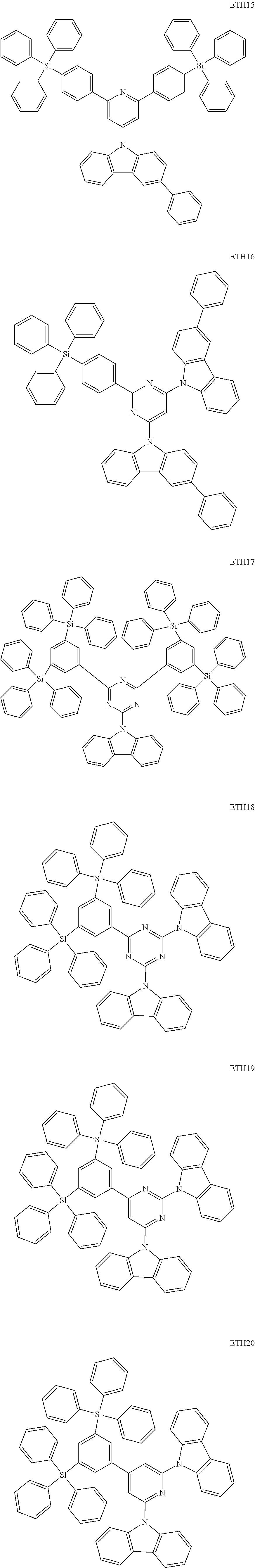

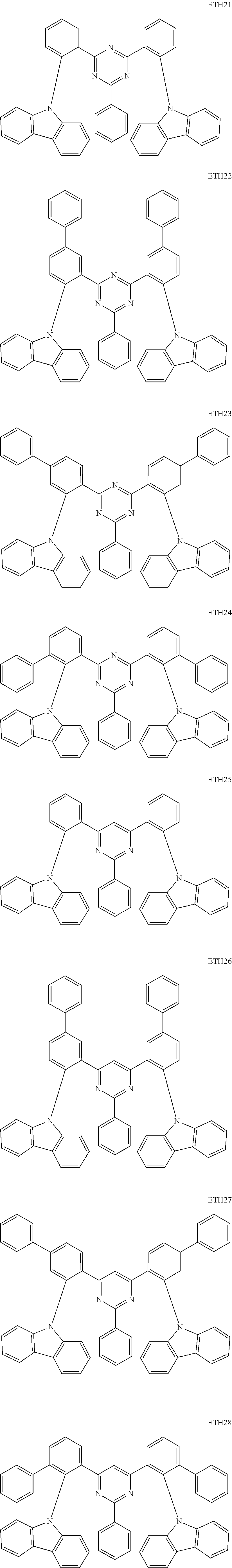

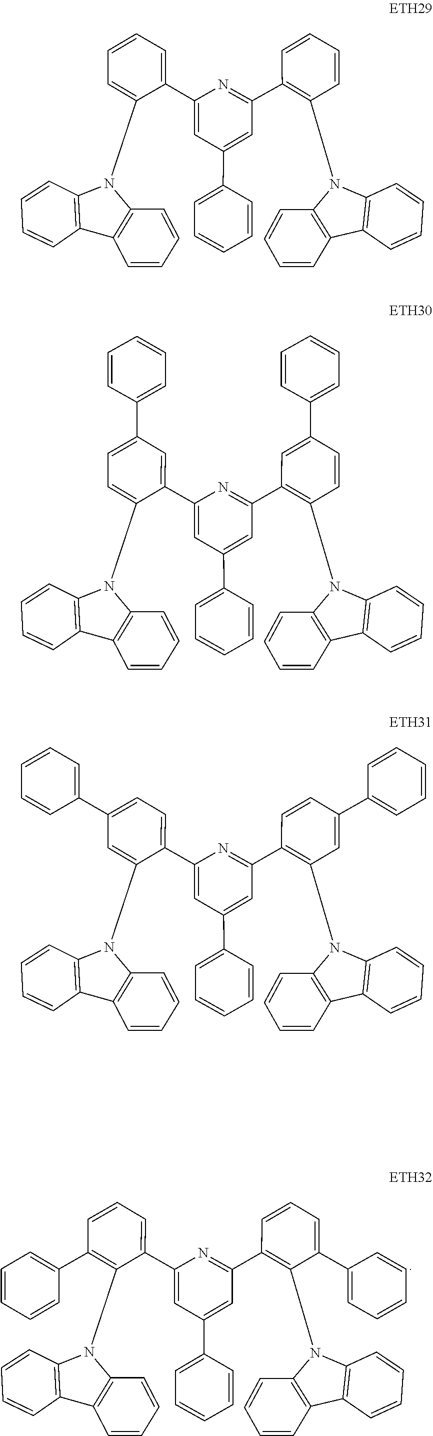 [SMILES: c1ccc(-c2cc(-c3ccccc3-n3c4ccccc4c4ccccc43)nc(-c3ccccc3-n3c4ccccc4c4ccccc43)c2)cc1 ; c1ccc(-c2cc(-c3cc(-c4ccccc4)ccc3-n3c4ccccc4c4ccccc43)nc(-c3cc(-c4ccccc4)ccc3-n3c4ccccc4c4ccccc43)c2)cc1 ; c1ccc(-c2cc(-c3ccc(-c4ccccc4)cc3-n3c4ccccc4c4ccccc43)nc(-c3ccc(-c4ccccc4)cc3-n3c4ccccc4c4ccccc43)c2)cc1 ; c1ccc(-c2cc(-c3cccc(-c4ccccc4)c3-n3c4ccccc4c4ccccc43)nc(-c3cccc(-c4ccccc4)c3-n3c4ccccc4c4ccccc43)c2)cc1]

In an embodiment, the second host may include a compound represented by Formula 7:

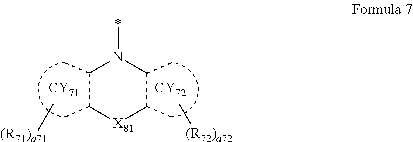

wherein, in Formula 7,
rings CY₇₁ and CY₇₂ may each independently be a C₅-C₆₀ carbocyclic group or a C₁-C₆₀ heterocyclic group,
X₈₁ may be a single bond, O, S, N(R₈₁), B(R₈₁), C(R₈₁ₐ)(R_{81b}), or Si(R₈₁ₐ)(R_{81b}),
R₇₁, R₇₂, R₈₁, R₈₁ₐ, and R_{81b} are as described for R₁ elsewhere herein, and
a71 and a72 may each independently be an integer from 0 to 10.

In an embodiment, in Formula 7, rings CY₇₁ and CY₇₂ may each independently include i) a first ring, ii) a second ring, iii) a condensed ring in which two or more first rings are condensed with each other, iv) a condensed ring in which two or more second rings are condensed with each other, or v) a condensed ring in which at least one first ring and at least one second ring are condensed with each other, and
the first ring may include a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group.

The second ring may include a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, or an azadibenzofuran group.

In an embodiment, rings CY₇₁ and CY₇₂ in Formula 7 may each independently include a benzene group, a naphthalene group, an anthracene group, a phenanthrene group, a triphenylene group, a pyrene group, a chrysene group, a cyclopentadiene group, a 1,2,3,4-tetrahydronaphthalene group, a thiophene group, a furan group, an indole group, a benzoborole group, a benzophosphole group, an indene group, a benzosilole group, a benzogermole group, a benzothiophene group, a benzoselenophene group, a benzofuran group, a carbazole group, a dibenzoborole group, a dibenzophosphole group, a fluorene group, a dibenzosilole group, a dibenzogermole group, a dibenzothiophene group, a dibenzoselenophene group, a dibenzofuran group, a dibenzothiophene 5-oxide group, a 9H-fluorene-9-one group, a dibenzothiophene 5,5-dioxide group, an azaindole group, an azabenzoborole group, an azabenzophosphole group, an azaindene group, an azabenzosilole group, an azabenzogermole group, an azabenzothiophene group, an azabenzoselenophene group, an azabenzofuran group, an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene 5-oxide group, an aza-9H-fluorene-9-one group, an dioxide group, a pyridine group, a pyrimidine azadibenzothiophene 5,5-group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isoxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a 5,6,7,8-tetrahydroisoquinoline group, or a 5,6,7,8-tetrahydroquinoline group.

In an embodiment, the second host may include a compound represented by one of Formulae 7-1 to 7-5:

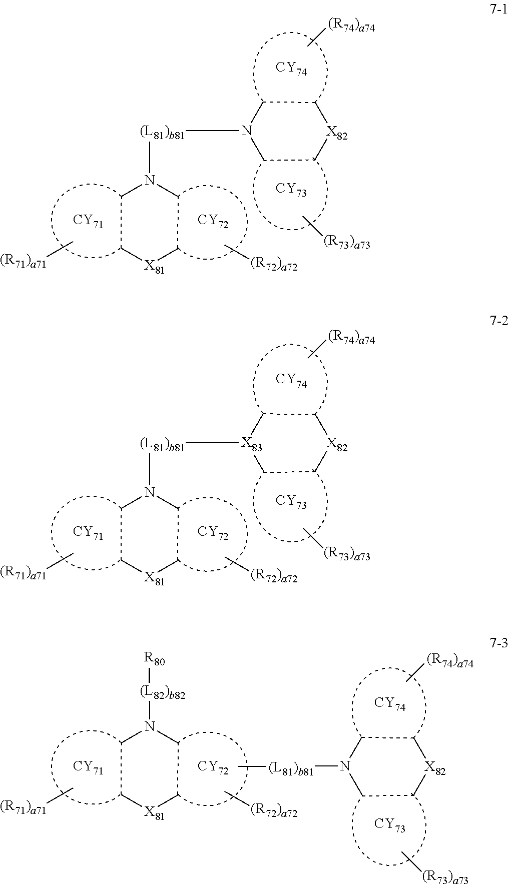

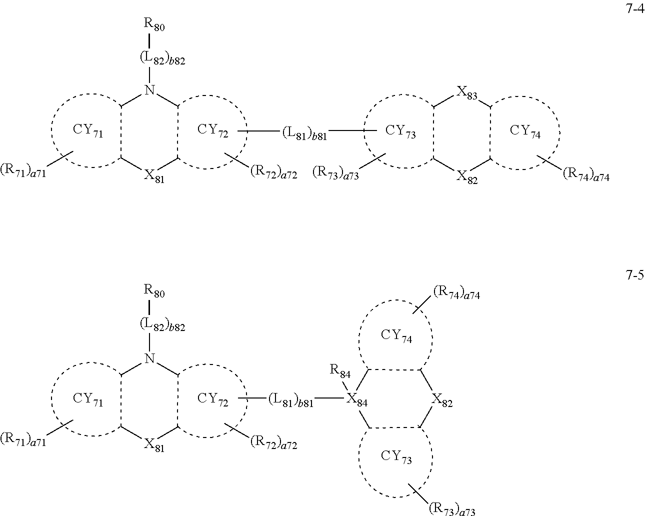

wherein, in Formulae 7-1 to 7-5,
ring CY₇₁, ring CY₇₂, X₈₁, R₇₁, R₇₂, a71, and a72 are as described elsewhere herein,
ring CY₇₃, ring CY₇₄, R₇₃, R₇₄, a73, and a74 are as described in connection with ring CY₇₁, ring CY₇₂, R₇₁, R₇₂, a71, and a72,
L₈₁ and L₈₂ may each independently be selected from *—C(Q₄)(Q₅)-*′, *—Si(Q₄)(Q₅)-*′, a substituted or unsubstituted C₅-C₃₀ carbocyclic group, and a substituted or unsubstituted C₁-C₃₀ heterocyclic group, and Q₄ and Q₅ are as described for Q₁,
b81 and b82 may each be an integer from 0 to 5, wherein, when b81 is 0, *-(L₈₁)_{b81}-*′ may be a single bond, when b81 is 2 or more, two or more of L₈₁(s) may be identical to or different from each other, when b82 is 0, *-(L₈₂)_{b82}-*′ may be a single bond, and when b82 is 2 or more, two or more of L₈₂(s) may be identical to or different from each other,
X₈₂ may be a single bond, O, S, N(R₈₂), B(R₈₂), C(R₈₂ₐ)(R_{82b}), or Si(R₈₂ₐ)(R_{82b}),
X₈₃ may be a single bond, O, S, N(R₈₃), B(R₈₃), C(R₈₃ₐ)(R_{83b}), or Si(R₈₃ₐ)(R_{83b}),
each of X₈₂ and X₈₃ in Formulae 7-2 and 7-4 may not be a single bond,
X₈₄ may be C or Si,
R₈₀, R₈₂, R₈₃, R₈₂ₐ, R_{82b}, R₈₃ₐ, R_{83b}, and R₈₄ are as described for R₈₁, and
* and *′ each indicate a binding site to a neighboring atom (i.e., a point of attachment).

In some embodiments,
a moiety represented by

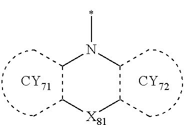

in Formulae 7-1 and 7-2 may be a group represented by one of Formulae CY71-1(1) to CY71-1(8),
a moiety represented by

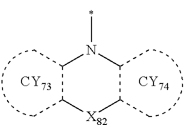

in Formulae 7-1 and 7-3 may be a group represented by one of Formulae CY71-2(1) to CY71-2(8),
a moiety represented by,

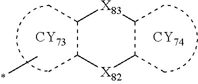

in Formulae 7-2 and 7-4 may be a group represented by one of Formulae CY71-3(1) to CY71-3(32),
a moiety represented by

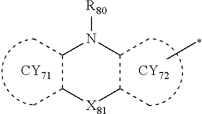

in Formulae 7-3 to 7-5 may be a group represented by one of Formulae CY71-4(1) to CY71-4(32), and
a moiety represented by

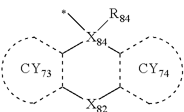

in Formula 7-5 may be a group represented by one of Formulae CY71-5(1) to CY71-5(8):

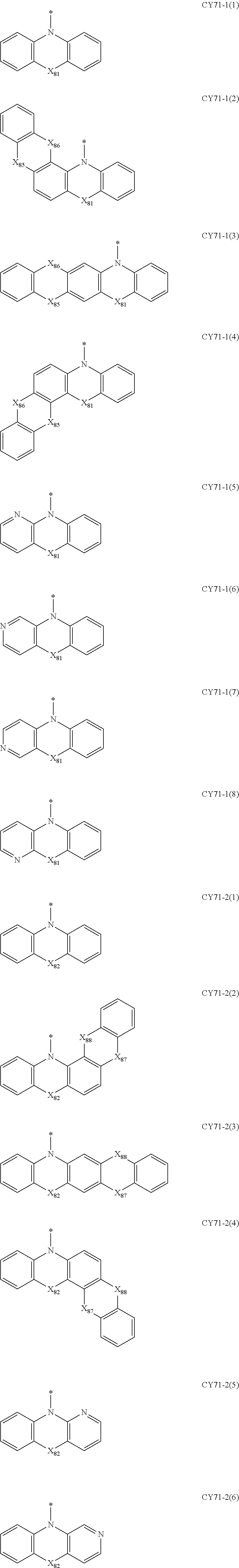

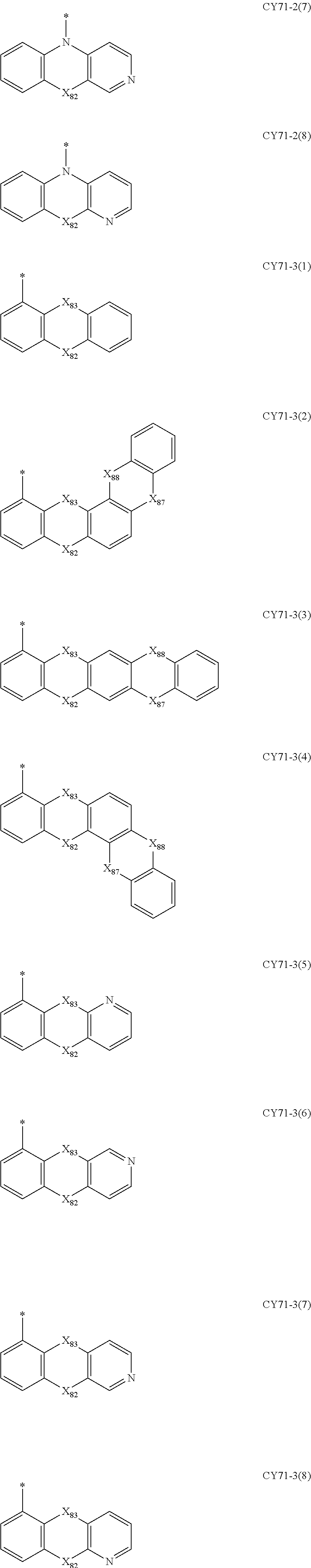

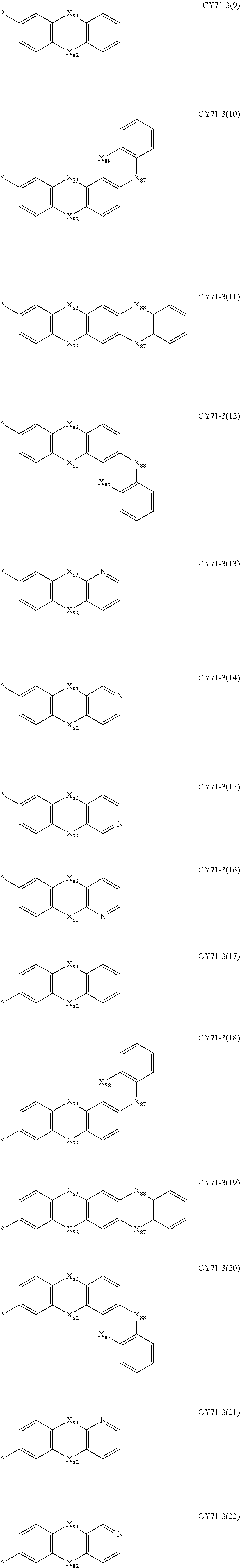

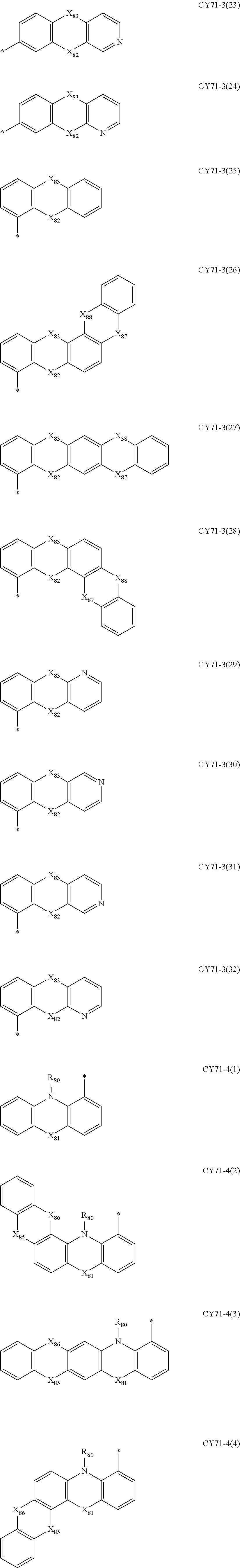

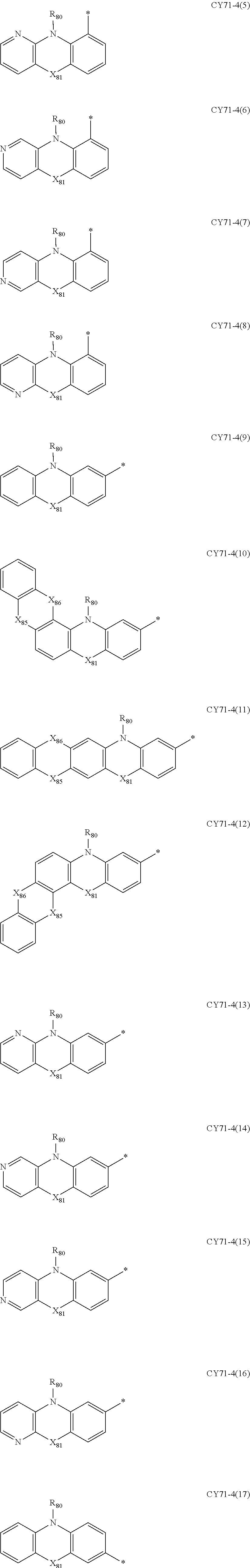

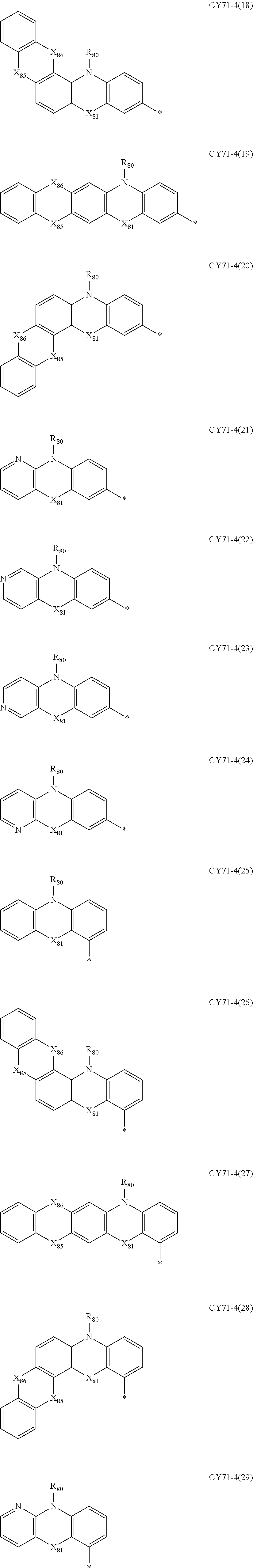

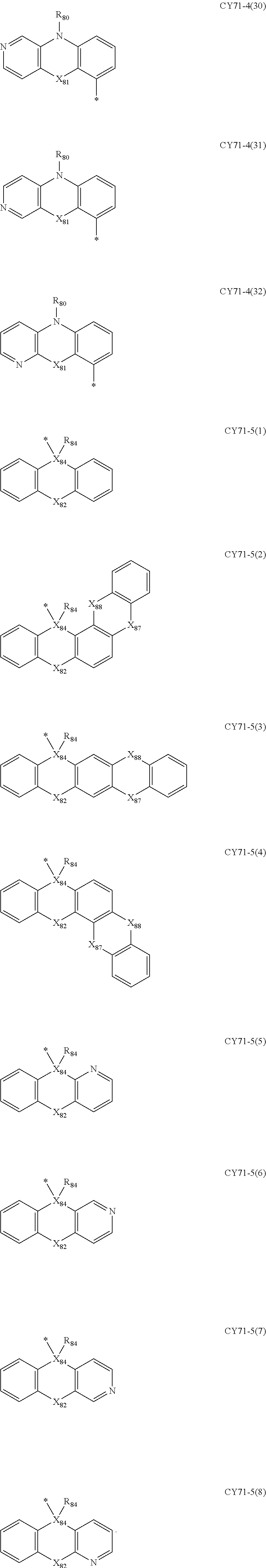

In Formulae CY71-1(1) to CY71-1(8), CY71-2(1) to CY71-2(8), CY71-3(1) to CY71-3(32), CY71-4(1) to CY71-4(32), and CY71-5(1) to CY71-5(8),
X₈₁ to X₈₄, R₈₀, and R₈₄ are as described elsewhere herein,
X₈₅ may be a single bond, O, S, N(R₈₅), B(R₈₅), C(R₈₅ₐ)(R_{85b}), or Si(R₈₅ₐ)(R_{85b}),
X₈₆ may be a single bond, O, S, N(R₈₆), B(R₈₆), C(R₈₆ₐ)(R_{86b}), or Si(R₈₆ₐ)(R_{86b}),
each of X₈₅ and X₈₆ in Formulae CY71-1(1) to CY71-1(8) and CY71-4(1) to CY71-4(32) is not a single bond,
X₈₇ may be a single bond, O, S, N(R₈₇), B(R₈₇), C(R₈₇ₐ)(R_{87b}), or Si(R₈₇ₐ)(R_{87b}),
X₈₈ may be a single bond, O, S, N(R₈₈), B(R₈₈), C(R₈₈ₐ)(R_{88b}), or Si(R₈₈ₐ)(R_{88b}),
each of X₈₇ and X₈₈ in Formulae CY71-2(1) to CY71-2(8), CY71-3(1) to CY71-3(32) and CY71-5(1) to CY71-5(8) may not be a single bond, and
R₈₅ to R₈₈, R₈₅ₐ, R_{85b}, R₈₆ₐ, R_{86b}, R₈₇ₐ, R_{87b}, R₈₈ₐ and R_{88b} are as described for R₈₁.

In an embodiment, the second host may include at least one of compounds represented by Formulae HTH1 to HTH40:

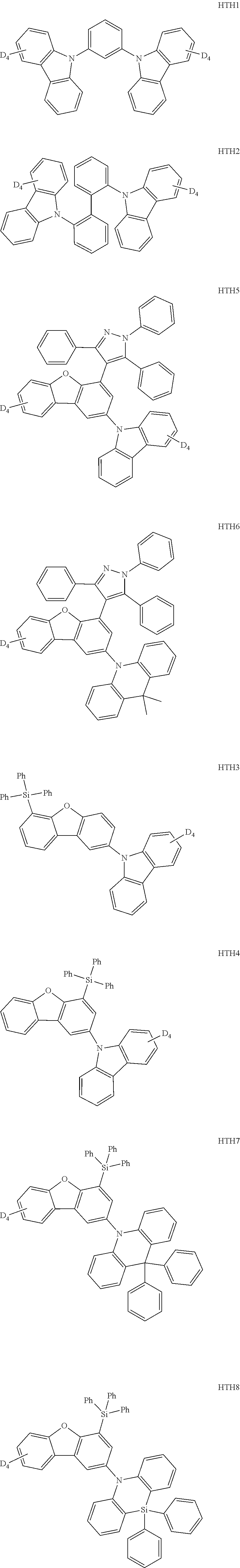

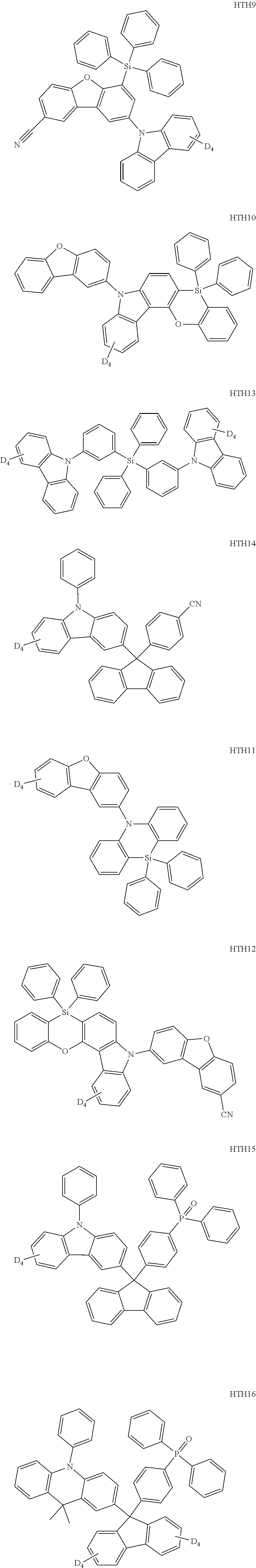

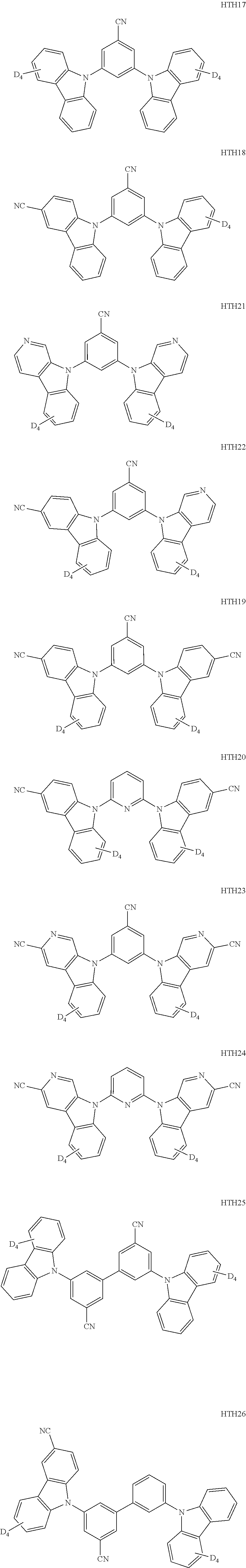

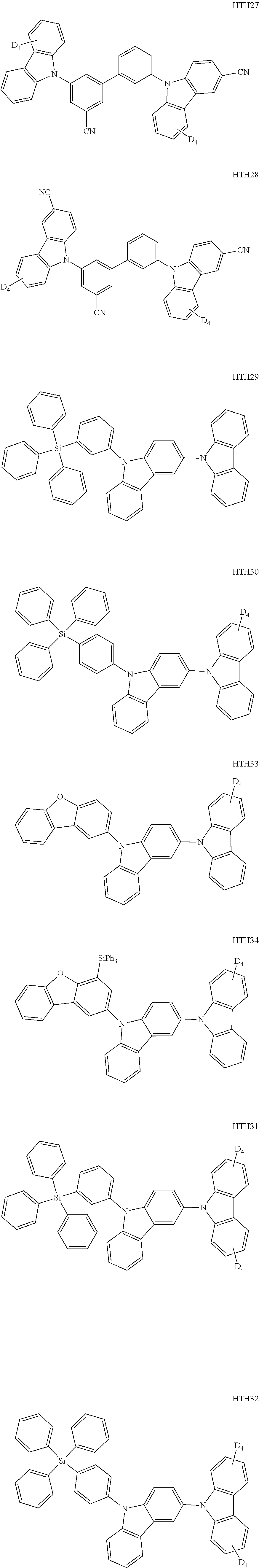

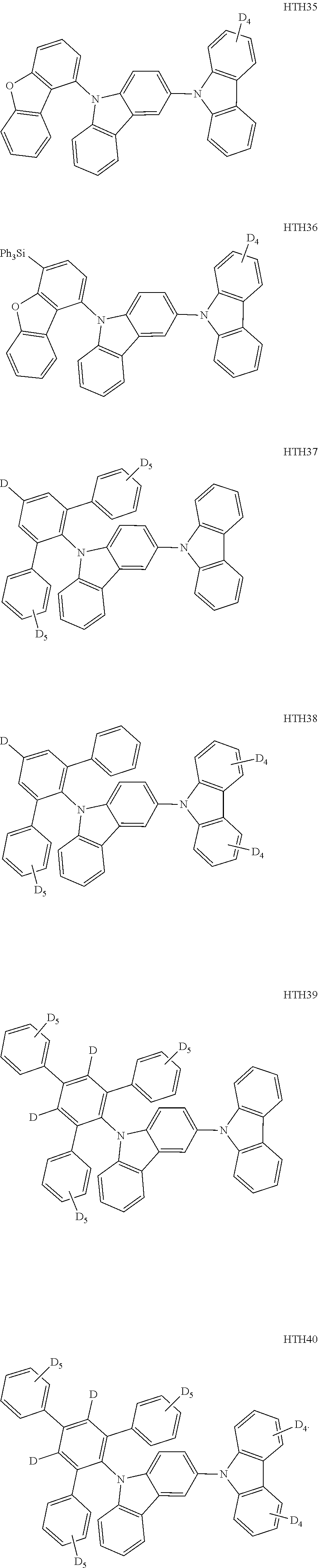

In the light-emitting device according to an embodiment, the first host and the second host may form an exciplex (i.e., excited state complex). In some embodiments, the organometallic compound and the first host may not form an exciplex, and/or the organometallic compound and the second host may not form an exciplex.

Another aspect of the present disclosure relates to an electronic apparatus including the light-emitting device. The electronic apparatus may include a thin-film transistor. For example, the electronic apparatus may include a thin-film transistor including a source electrode and a drain electrode, wherein the first electrode of the light-emitting device may be electrically connected to the source electrode or the drain electrode. In an embodiment, the electronic apparatus may include a color filter, a color conversion layer, a touch screen layer, a polarizing layer, or a combination thereof. More details on the electronic apparatus are disclosed elsewhere herein.

Another aspect of the present disclosure relates to an electronic equipment including the light-emitting device. The electronic equipment may include a flat panel display, a curved display, a computer monitor, a medical monitor, a television, a billboard, an indoor or outdoor light and/or light for signal, a head-up display, a fully or partially transparent display, a flexible display, a rollable display, a foldable display, a stretchable display, a laser printer, a telephone, a portable phone, a tablet personal computer, a phablet, a personal digital assistant (PDA), a wearable device, a laptop computer, a digital camera, a camcorder, a viewfinder, a micro display, a three-dimensional (3D) display, a virtual reality or augmented reality display, a vehicle, a video wall with multiple displays tiled together, a theater or stadium screen, a phototherapy device, and/or a signboard. More details on the electronic equipment are disclosed elsewhere herein.

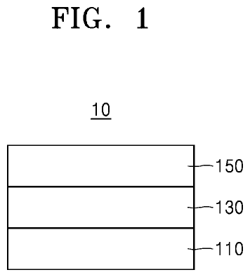

### Description of FIG. 1

FIG. 1 is a schematic cross-sectional view of a light-emitting device 10 according to an embodiment. The light-emitting device 10 includes a first electrode 110, an interlayer 130, and a second electrode 150.

Hereinafter, the structure of the light-emitting device 10 according to an embodiment and a method of manufacturing the light-emitting device 10 will be described with reference to FIG. 1.

### First Electrode 110

In FIG. 1, a first substrate may be located at (e.g., below or under), the first electrode 110 and/or a second substrate may be located at (e.g., on or above), the second electrode 150. The first and/or second substrate may be a glass substrate or a plastic substrate. In some embodiments, the first and/or second substrate may be a flexible substrate, and may include plastic(s) with excellent or suitable heat resistance and durability, such as polyimide, polyethylene terephthalate (PET), polycarbonate, polyethylene napthalate, polyarylate (PAR), polyetherimide, or a combination thereof.

The first electrode 110 may be formed by, for example, depositing or sputtering a material for forming the first electrode 110 on the substrate. When the first electrode 110 is an anode, a material for forming the first electrode 110 may be a high work-function material that facilitates injection of holes. The term “high work-function material” as utilized herein refers to a substance (e.g., a metal or metal alloy) that requires a relatively high amount of energy to emit electrons from its surface.

The first electrode 110 may be a reflective electrode, a semi-transmissive electrode, or a transmissive electrode. When the first electrode 110 is a transmissive electrode, a material for forming the first electrode 110 may include indium tin oxide (ITO), indium zinc oxide (IZO), tin oxide (SnO₂), zinc oxide (ZnO), or a combination thereof. In some embodiments, when the first electrode 110 is a semi-transmissive electrode or a reflective electrode, a material for forming the first electrode 110 may include magnesium (Mg), silver (Ag), aluminum (Al), aluminum-lithium (Al—Li), calcium (Ca), magnesium-indium (Mg—In), magnesium-silver (Mg—Ag), or a combination thereof.

In some embodiments, the first electrode 110 may have a single-layered structure including a single layer, or a multi-layered structure that has a plurality of layers. For example, the first electrode 110 may have a three-layered structure of ITO/Ag/ITO.

### Interlayer 130

The interlayer 130 may be located at (e.g., on or above), the first electrode 110. The interlayer 130 may include an emission layer.

The interlayer 130 may further include a hole transport region located between the first electrode 110 and the emission layer, and an electron transport region located between the emission layer and the second electrode 150.

In some embodiments, the interlayer 130 may include one or more suitable organic materials, a metal-containing compound such as an organometallic compound, an inorganic material such as quantum dots, and/or the like.

In some embodiments, the interlayer 130 may include, i) two or more emitting units located between (e.g., sequentially stacked between) the first electrode 110 and the second electrode 150, and ii) a charge generation layer located in between respective emitting units (e.g., the two emitting units). In some embodiments, the light-emitting device 10 that has the interlayer 130 that includes emitting units and a charge generation layer may be referred to as a tandem light-emitting device.

### Hole Transport Region in Interlayer 130

In some embodiments, the hole transport region may include: i) a single-layered structure that includes (e.g., consists of) a single layer including (e.g., consisting of) a single type or kind of material, ii) a single-layered structure that includes (e.g., consists of) a single layer that includes more than one type or kind of materials, or iii) a multi-layered structure including multiple layers that include more than one type or kind of materials.

In some embodiments, the hole transport region may include a hole injection layer, a hole transport layer, an emission auxiliary layer, an electron blocking layer, and/or a combination thereof.

For example, the hole transport region may have a multi-layered structure including a hole injection layer/hole transport layer structure, a hole injection layer/hole transport layer/emission auxiliary layer structure, a hole injection layer/emission auxiliary layer structure, a hole transport layer/emission auxiliary layer structure, and/or a hole injection layer/hole transport layer/electron blocking layer structure. In some embodiments, the layers of each structure may be stacked sequentially starting from the first electrode 110.

In some embodiments, the hole transport region may include the amine-containing compound disclosed herein, a compound represented by Formula 201, a compound represented by Formula 202, and/or a combination thereof:

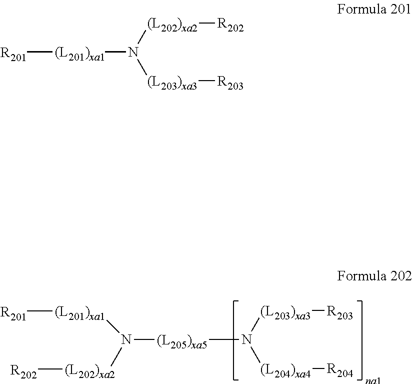

wherein, in Formulae 201 and 202,
L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
L₂₀₅ may be *—O—*′, *—S—*′, *—N(Q₂₀₁)-*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
xa1 to xa4 may each independently be an integer from 0 to 5,
xa5 may be an integer from 1 to 10,
R₂₀₁ to R₂₀₄ and Q₂₀₁ may each independently be a C₃-C₆₀ carbocyclic group unsubstituted or substituted with at least one R₁₀ₐ, or a C₁-C₆₀ heterocyclic group unsubstituted or substituted with at least one R₁₀ₐ,
R₂₀₁ and R₂₀₂ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group (for example, a carbazole group and/or the like) unsubstituted or substituted with at least one R₁₀ₐ (for example, Compound HT16),
R₂₀₃ and R₂₀₄ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group unsubstituted or substituted with at least one R₁₀ₐ, and
na1 may be an integer from 1 to 4.

For example, each of Formulae 201 and 202 may include at least one of groups represented by Formulae CY201 to CY217:

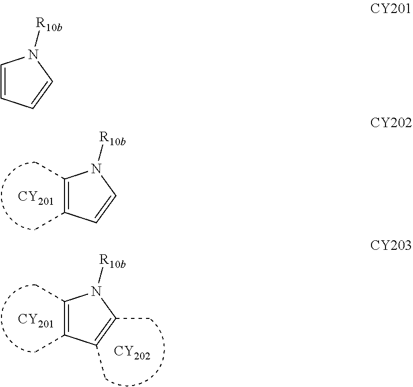

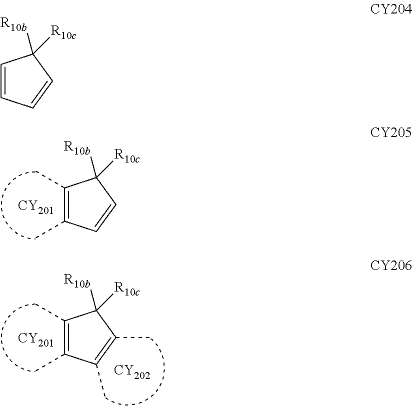

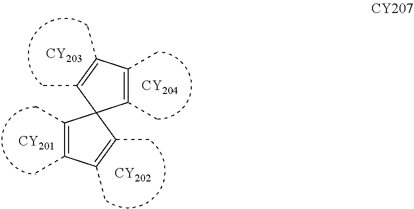

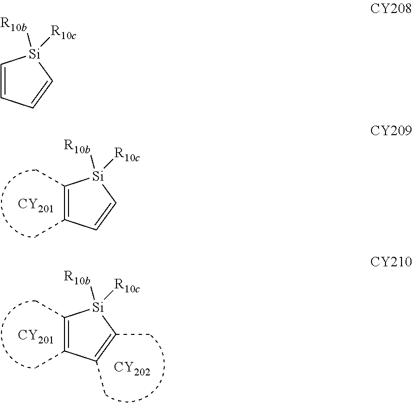

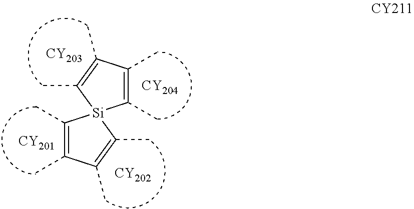

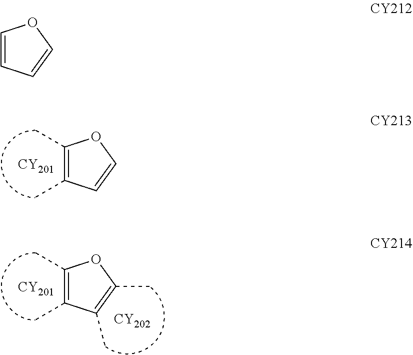

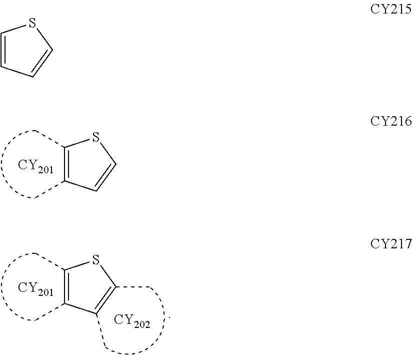

In Formulae CY201 to CY217, R_{10b} and R_{10c} may each be as described for R₁₀ₐ, ring CY201 to ring CY204 may each independently be a C₃-C₂₀ carbocyclic group or a C₁-C₂₀ heterocyclic group, and at least one hydrogen in Formulae CY201 to CY217 may be replaced with R₁₀ₐ as described above.

In an embodiment, ring CY201 to ring CY204 in Formulae CY201 to CY217 may each independently be a benzene group, a naphthalene group, a phenanthrene group, or an anthracene group.

In some embodiments, each of Formulae 201 and 202 may include at least one of the groups represented by Formulae CY201 to CY203.

In some embodiments, Formula 201 may include at least one of the groups represented by Formulae CY201 to CY203 and at least one of the groups represented by Formulae CY204 to CY217.

In some embodiments, in Formula 201, xa1 may be 1, R₂₀₁ may be a group represented by one of Formulae CY201 to CY203, xa2 may be 0, and R₂₀₂ may be a group represented by one of Formulae CY204 to CY207.

In some embodiments, each of Formulae 201 and 202 may not include (e.g., may exclude), a group represented by one of Formulae CY201 to CY203.

In some embodiments, each of Formulae 201 and 202 may not include (e.g., may exclude), a group represented by one of Formulae CY201 to CY203, and may include at least one of the groups represented by Formulae CY204 to CY217.

In some embodiments, each of Formulae 201 and 202 may not include (e.g., may exclude), a group represented by one of Formulae CY201 to CY217.

In an embodiment, the hole transport region may include at least one of Compounds HT1 to HT46, 4,4′,4″-tris[(3-m ethylphenyl)phenylamino]triphenylamine (m-MTDATA), 1-N,1-N-bis[4-(diphenylamino)phenyl]-4-N,4-N-diphenylbenzene-1,4-diamine (TDATA), 4,4′,4″-tris[2-naphthyl(phenyl)amino]triphenylamine (2-TNATA), N,N′-bis(naphthalen-1-yl)-N,N′-bis(phenyl)benzidine (NPB or NPD), β-NPB, N,N′-bis(3-methylphenyl)-N,N′-diphenylbenzidine (TPD), Spiro-TPD, Spiro-NPB, methylated NPB, 1,1-Bis[(di-4-tolylamino)phenyl]cyclohexane (TAPC), N,N,N′,N′-Tetra-(3-methylphenyl)-3,3′-dimethylbenzidine (HMTPD), 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), or a combination thereof:

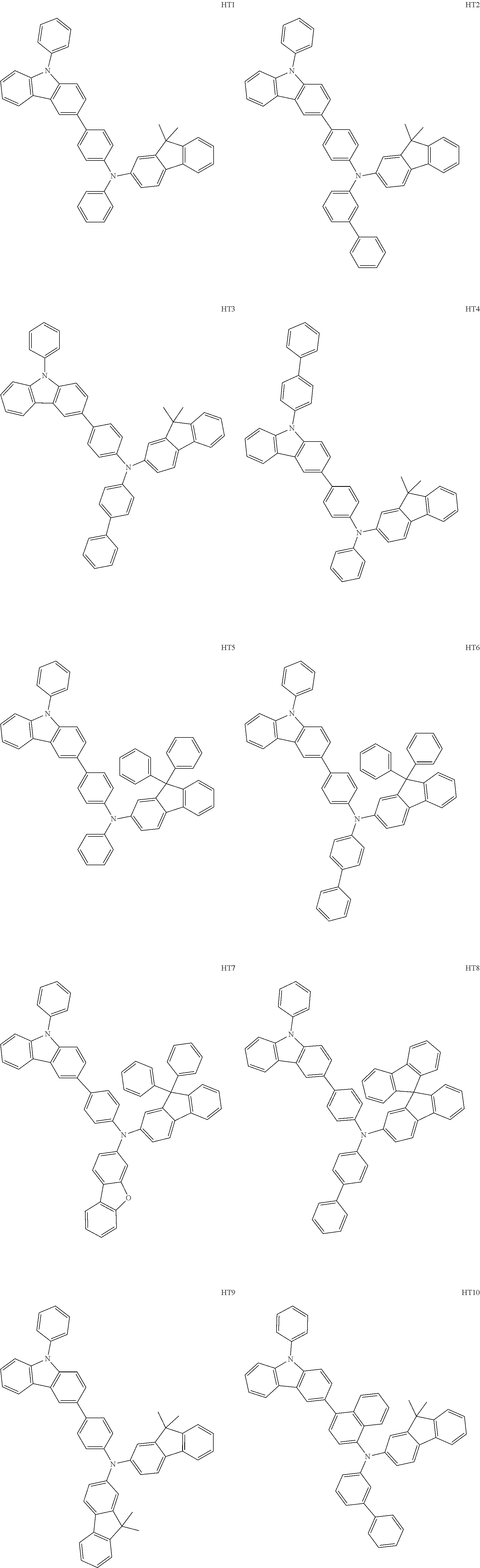

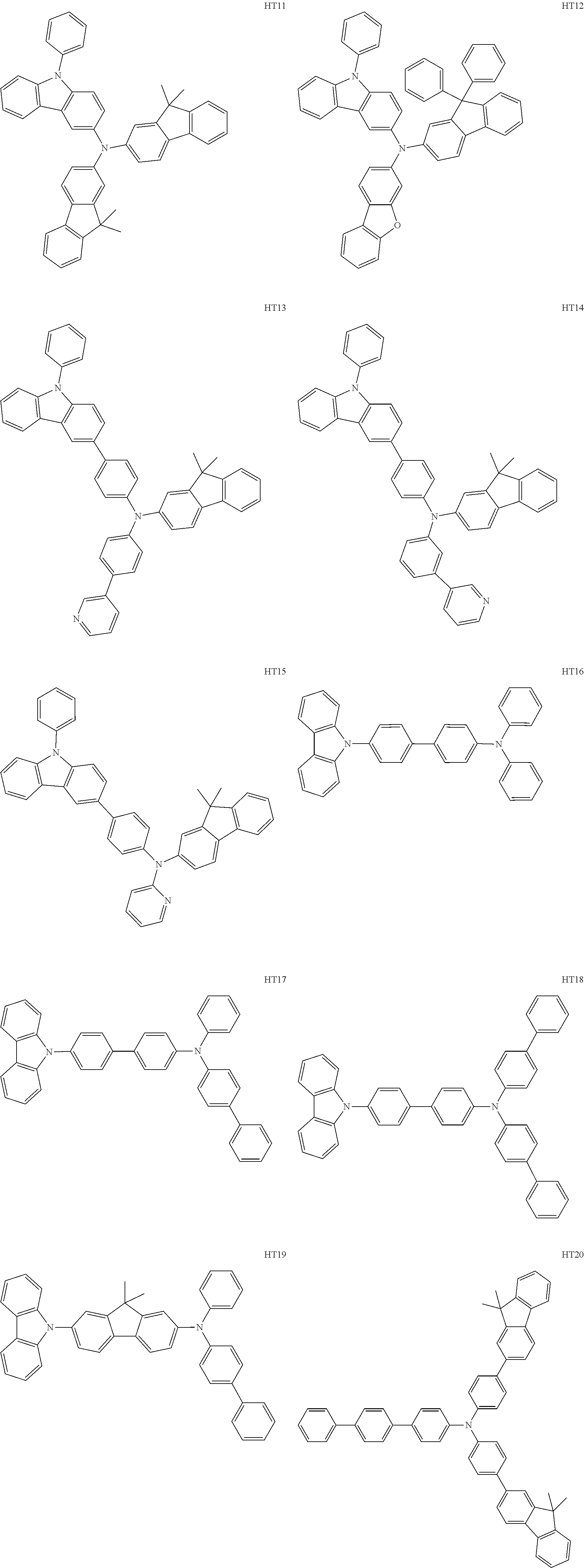

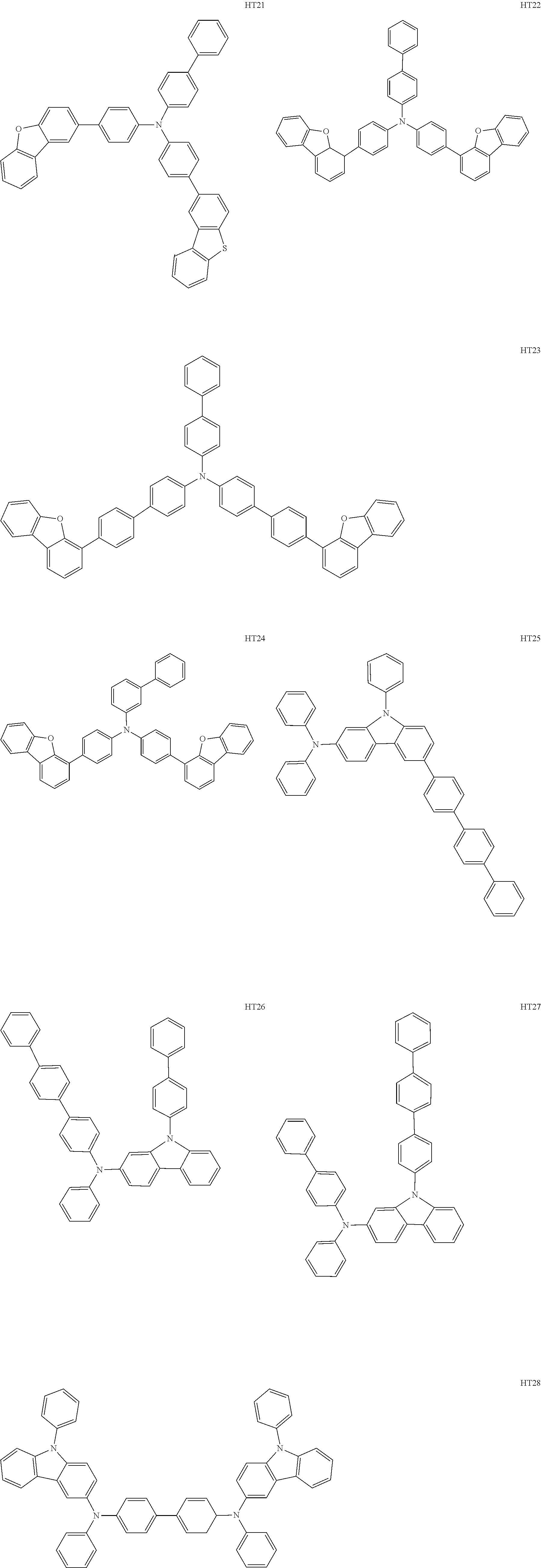

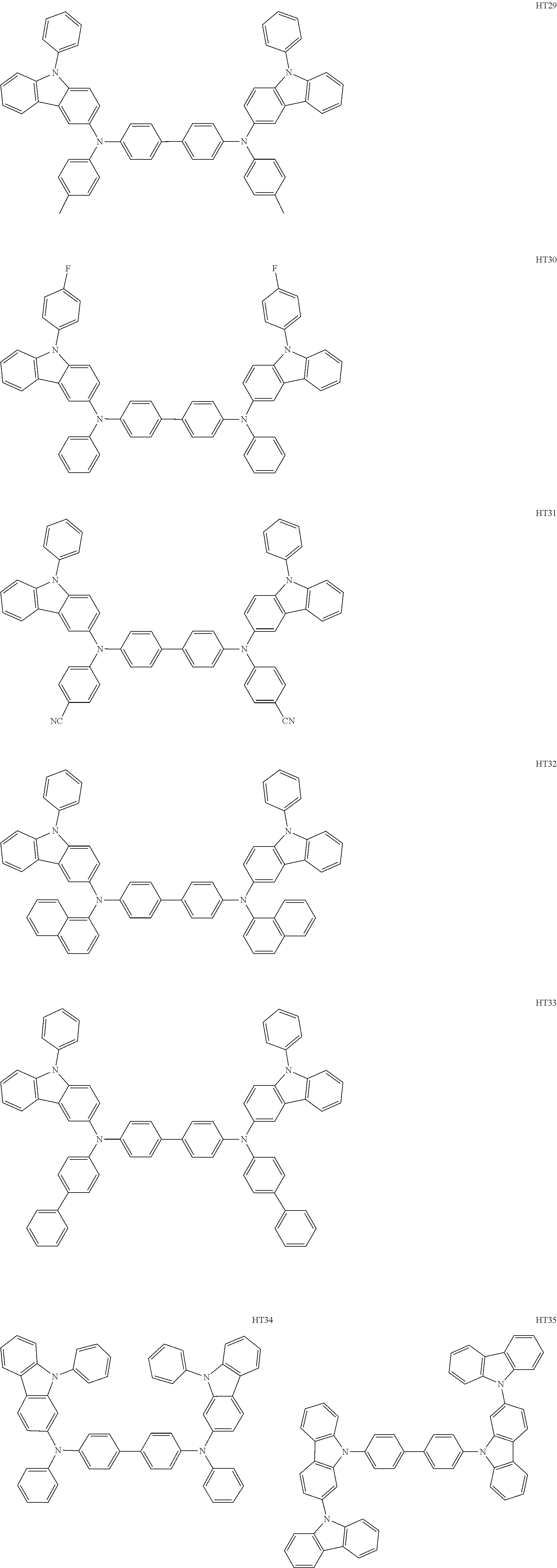

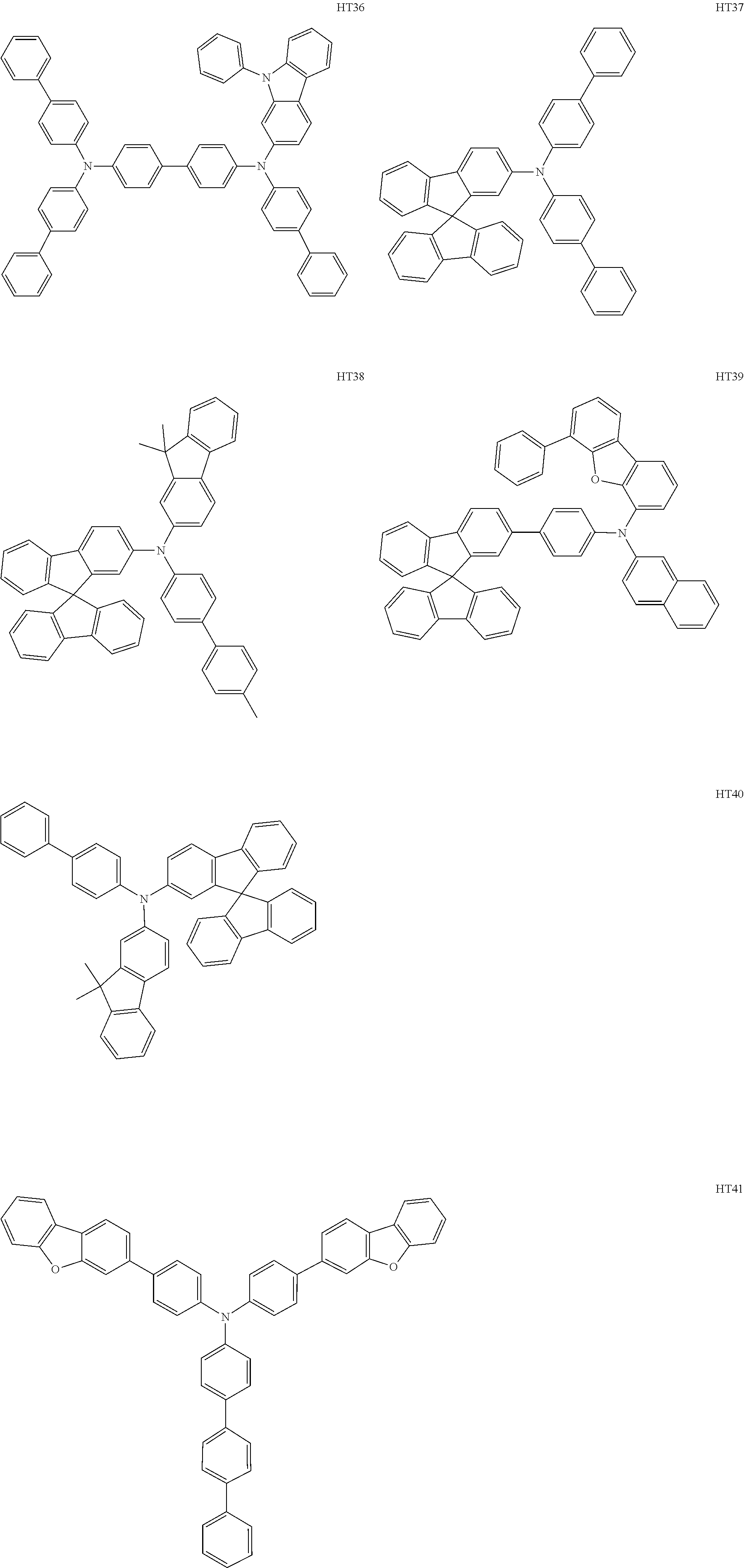

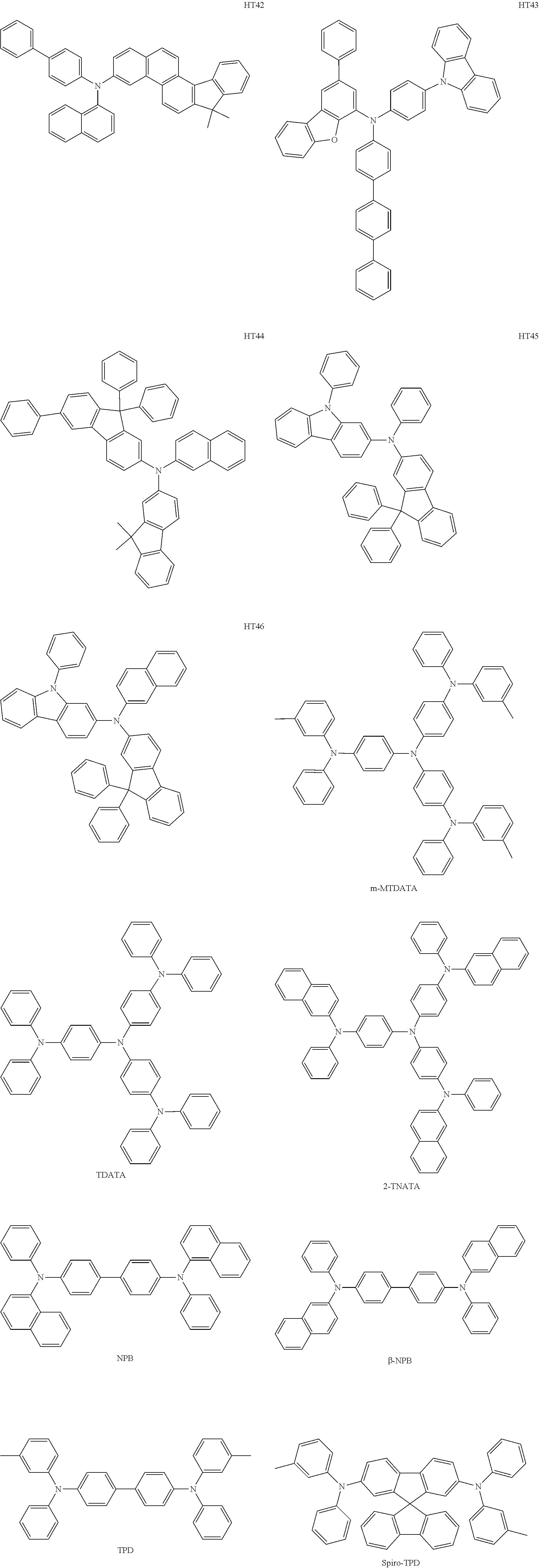

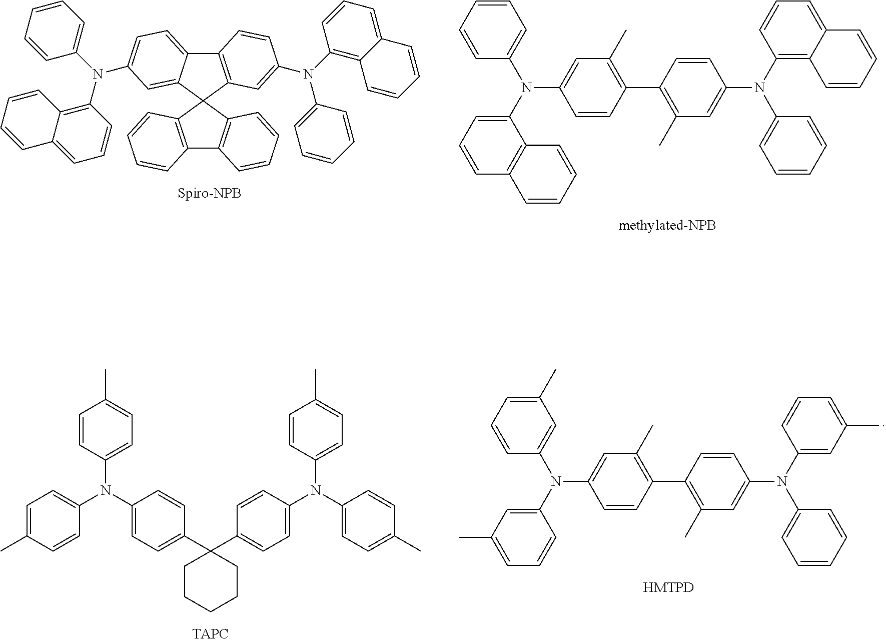

A thickness of the hole transport region may be about 50 Å to about 10,000 Å (angstroms), for example, about 100 Å to about 4,000 Å. When the hole transport region includes a hole injection layer, a hole transport layer, or a combination thereof, a thickness of the hole injection layer may be about 100 Å to about 9,000 Å, for example, about 100 Å to about 1,000 Å, and a thickness of the hole transport layer may be about 50 Å to about 2,000 Å, for example, about 100 Å to about 1,500 Å. In an embodiment, thicknesses of the hole transport region, the hole injection layer, and the hole transport layer within these ranges are associated with satisfactory hole transporting characteristics being obtained without, or in the absence of, a substantial increase in driving voltage.

In an embodiment, the emission auxiliary layer may increase light-emission efficiency by compensating for an optical resonance distance according to the wavelength of light emitted by an emission layer. In an embodiment, the electron blocking layer may block or reduce the leakage of electrons from an emission layer to a hole transport region. Materials that may be included in the hole transport region may be included in the emission auxiliary layer and the electron blocking layer.

p-Dopant

The hole transport region may further include a charge-generation material for the improvement of conductive properties. The charge-generation material may be uniformly or non-uniformly dispersed in the hole transport region (for example, in the form of a single layer that includes a charge-generation material).

In some embodiments, the charge-generation material may include a p-dopant. In some embodiments, the lowest unoccupied molecular orbital (LUMO) energy level of the p-dopant may be −3.5 eV or less.

In some embodiments, the p-dopant may include a quinone derivative, a cyano group-containing compound, a compound including element EL1 and element EL2, or a combination thereof.

Non-limiting examples of the quinone derivative are TCNQ, F4-TCNQ, and/or the like.

Non-limiting examples of the cyano group-containing compound are HAT-CN and a compound represented by Formula 221:

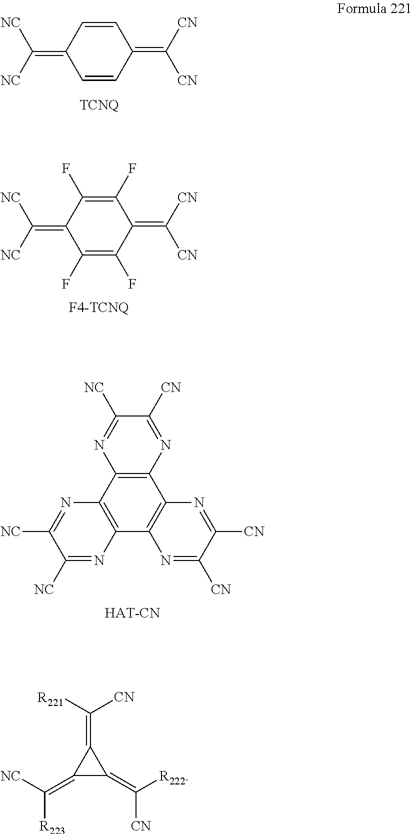

In Formula 221,
R₂₂₁ to R₂₂₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, and at least one of R₂₂₁ to R₂₂₃ may be a C₃-C₆₀ carbocyclic group and/or a C₁-C₆₀ heterocyclic group, each substituted with a cyano group; —F; —Cl; —Br; —I; a C₁-C₂₀ alkyl group substituted with a cyano group, —F, —Cl, —Br, —I, or a combination thereof; or a combination thereof.

In the compound including element EL1 and element EL2, element EL1 may be metal, metalloid, or a combination thereof, and element EL2 may be non-metal, metalloid, or a combination thereof. Non-limiting examples of the metal are an alkali metal (for example, lithium (Li), sodium (Na), potassium (K), rubidium (Rb), cesium (Cs), etc.); an alkaline earth metal (for example, beryllium (Be), magnesium (Mg), calcium (Ca), strontium (Sr), barium (Ba), etc.); a transition metal (for example, titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), etc.); a post-transition metal (for example, zinc (Zn), indium (In), tin (Sn), etc.); and a lanthanide metal (for example, lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium I, ytterbium (Yb), lutetium (Lu), etc.). Non-limiting examples of the metalloid are silicon (Si), antimony (Sb), and tellurium (Te). Non-limiting examples of the non-metal are oxygen (O) and halogen (for example, F, Cl, Br, I, etc.).

Non-limiting examples of the compound including element EL1 and element EL2 are metal oxide, metal halide (for example, metal fluoride, metal chloride, metal bromide, or metal iodide), metalloid halide (for example, metalloid fluoride, metalloid chloride, metalloid bromide, or metalloid iodide), metal telluride, or a combination thereof.

Non-limiting examples of the metal oxide are tungsten oxide (for example, WO, W₂O₃, WO₂, WO₃, W₂O₅, etc.), vanadium oxide (for example, VO, V₂O₃, VO₂, V₂O₅, etc.), molybdenum oxide (MoO, Mo₂O₃, MoO₂, MoO₃, Mo₂O₅, etc.), and rhenium oxide (for example, ReO₃, etc.).

Non-limiting examples of the metal halide are alkali metal halide, alkaline earth metal halide, transition metal halide, post-transition metal halide, and lanthanide metal halide.

Non-limiting examples of the alkali metal halide are LiF, NaF, KF, RbF, CsF, LiCl, NaCl, KCl, RbCl, CsCl, LiBr, NaBr, KBr, RbBr, CsBr, LiI, NaI, KI, RbI, CsI, and/or the like.

Non-limiting examples of the alkaline earth metal halide are BeF₂, MgF₂, CaF₂, SrF₂, BaF₂, BeCl₂, MgCl₂, CaCl₂, SrCl₂, BaCl₂, BeBr₂, MgBr₂, CaBr₂, SrBr₂, BaBr₂, BeI₂, MgI₂, CaI₂, SrI₂, BaI₂, and/or the like.

Non-limiting examples of the transition metal halide are titanium halide (for example, TiF₄, TiCl₄, TiBr₄, Til₄, etc.), zirconium halide (for example, ZrF₄, ZrCl₄, ZrBr₄, ZrI₄, etc.), hafnium halide (for example, HfF₄, HfCl₄, HfBr₄, HfI₄, etc.), vanadium halide (for example, VF₃, VCl₁₃, VBr₃, V₁₃, etc.), niobium halide (for example, NbF₃, NbCl₃, NbBr₃, NbI₃, etc.), tantalum halide (for example, TaF₃, TaCl₃, TaBr₃, TaI₃, etc.), chromium halide (for example, CrF₃, CrCl₃, CrBr₃, CrI₃, etc.), molybdenum halide (for example, MoF₃, MoCl₃, MoBr₃, MoI₃, etc.), tungsten halide (for example, WF₃, WCl₃, WBr₃, WI₃, etc.), manganese halide (for example, MnF₂, MnCl₂, MnBr₂, MnI₂, etc.), technetium halide (for example, TcF₂, TcCl₂, TcBr₂, TcI₂, etc.), rhenium halide (for example, ReF₂, ReCl₂, ReBr₂, ReI₂, etc.), iron halide (for example, FeF₂, FeCl₂, FeBr₂, FeI₂, etc.), ruthenium halide (for example, RuF₂, RuCl₂, RuBr₂, RuI₂, etc.), osmium halide (for example, OsF₂, OsCl₂, OsBr₂, OsI₂, etc.), cobalt halide (for example, CoF₂, CoCl₂, CoBr₂, Cole, etc.), rhodium halide (for example, RhF₂, RhCl₂, RhBr₂, Rh₁₂, etc.), iridium halide (for example, IrF₂, IrCl₂, IrBr₂, Irl₂, etc.), nickel halide (for example, NiF₂, NiCl₂, NiBr₂, NiI₂, etc.), palladium halide (for example, PdF₂, PdCl₂, PdBr₂, PdI₂, etc.), platinum halide (for example, PtF₂, PtCl₂, PtBr₂, PtI₂, etc.), copper halide (for example, CuF, CuCl, CuBr, CuI, etc.), silver halide (for example, AgF, AgCl, AgBr, AgI, etc.), and gold halide (for example, AuF, AuCl, AuBr, AuI, etc.).

Non-limiting examples of the post-transition metal halide are zinc halide (for example, ZnF₂, ZnCl₂, ZnBr₂, ZnI₂, etc.), indium halide (for example, InI₃, etc.), and tin halide (for example, SnI₂, etc.).

Non-limiting examples of the lanthanide metal halide are YbF, YbF₂, YbF₃, SmF₃, YbCl, YbCl₂, YbCl₃ SmCl₃, YbBr, YbBr₂, YbBr₃ SmBr₃, YbI, YbI₂, YbI₃, and SmI₃.

A non-limiting example of the metalloid halide is antimony halide (for example, SbCl₅, etc.).

Non-limiting examples of the metal telluride are alkali metal telluride (for example, Li₂Te, Na₂Te, K₂Te, Rb₂Te, Cs₂Te, etc.), alkaline earth metal telluride (for example, BeTe, MgTe, CaTe, SrTe, BaTe, etc.), transition metal telluride (for example, TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, etc.), post-transition metal telluride (for example, ZnTe, etc.), lanthanide metal telluride (for example, LaTe, CeTe, PrTe, NdTe, PmTe, EuTe, GdTe, TbTe, DyTe, HoTe, ErTe, TmTe, YbTe, LuTe, etc.), and/or the like.

### Emission Layer in Interlayer 130

When the light-emitting device 10 is a full-color light-emitting device, the emission layer may be patterned into a red emission layer, a green emission layer, and/or a blue emission layer, according to a sub-pixel. In some embodiments, the emission layer may have a stacked structure of two or more layers of a red emission layer, a green emission layer, and a blue emission layer. In some embodiments, the two or more layers contact each other and are configured to emit white light. In some embodiments, the two or more layers are separate from each other and are configured to emit white light. In some embodiments, the emission layer may include two or more materials of a red light-emitting material, a green light-emitting material, and a blue light-emitting material. In some embodiments, the two or more materials are mixed with each other in a single layer to emit white light.

The emission layer may include a host and/or a dopant.

The dopant may include a phosphorescent dopant, a fluorescent dopant, or a combination thereof. In some embodiments, the emission layer may include about 0.01 to about 15 parts by weight of the dopant, based on 100 parts by weight of the host.

In some embodiments, the emission layer may include a quantum dot. In some embodiments, the emission layer may include a plurality of quantum dots.

In some embodiments, the emission layer may include a delayed fluorescence material. The delayed fluorescence material may act as a host or a dopant in the emission layer. In some embodiments, the host includes the delayed fluorescence material. In some embodiments, the dopant includes the delayed fluorescence material.

A thickness of the emission layer may be about 100 Å to about 1,000 Å, for example, about 200 Å to about 600 Å. In an embodiment, the emission layer having the thickness within these ranges may be associated with excellent or suitable light-emission characteristics without, or in the absence of, a substantial increase in driving voltage.

### Host

In some embodiments, the host may include a compound represented by Formula 301:

[Ar₃₀₁]_{xb11}-[(L₃₀₁)_{xb1}-R₃₀₁]_{xb21} Formula 301

In Formula 301,
Ar₃₀₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xb11 may be 1, 2, or 3,
xb1 may be an integer from 0 to 5,
R₃₀₁ may be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-Cho alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),
xb21 may be an integer from 1 to 5, and
Q₃₀₁ to Q₃₀₃ are each as described for Q₁ elsewhere herein.

For example, when xb11 in Formula 301 is 2 or more, two or more of Ar₃₀₁(s) may be linked to each other via a single bond.

In some embodiments, the host may include a compound represented by Formula 301-1, a compound represented by Formula 301-2, or a combination thereof:

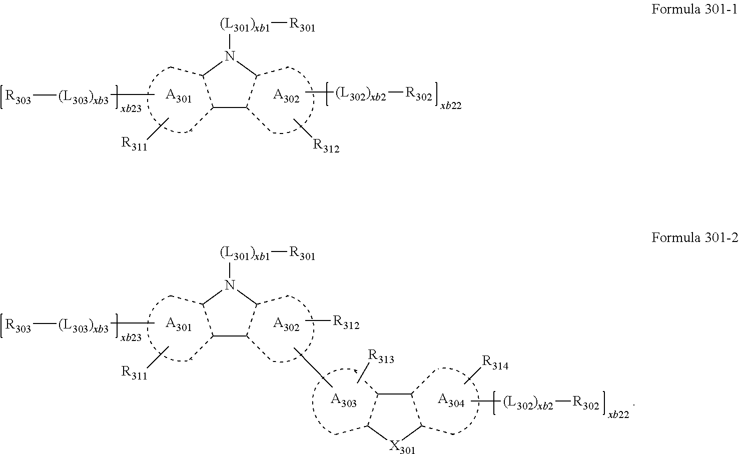

In Formulae 301-1 and 301-2,
ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
X₃₀₁ may be O, S, N—[(L₃₀₄)_{xb4}-R₃₀₄], C(R₃₀₄)(R₃₀₅), or Si(R₃₀₄)(R₃₀₅),
xb22 and xb23 may each independently be 0, 1, or 2,
L₃₀₁, xb1, and R₃₀₁ may each be as described elsewhere herein,
L₃₀₂ to L₃₀₄ may each independently be as described for L₃₀₁,
xb2 to xb4 may each independently be as described for xb1, and
R₃₀₂ to R₃₀₅ and R₃₁₁ to R₃₁₄ may each be as described for R₃₀₁.

In some embodiments, the host may include an alkali earth metal complex, a post-transition metal complex, or a combination thereof. For example, the host may include a Be complex (for example, Compound H55), an Mg complex, a Zn complex, or a combination thereof.

In some embodiments, the host may include at least one of Compounds H1 to H128, 9,10-di(2-naphthyl)anthracene (ADN), 2-methyl-9,10-bis(naphthalen-2-yl)anthracene (MADN), 9,10-di-(2-naphthyl)-2-t-butyl-anthracene (TBADN), 4,4′-bis(N-carbazolyl)-1,1′-biphenyl (CBP), 1,3-di-9-carbazolylbenzene (mCP), 1,3,5-tri(carbazol-9-yl)benzene (TCP), or a combination thereof:

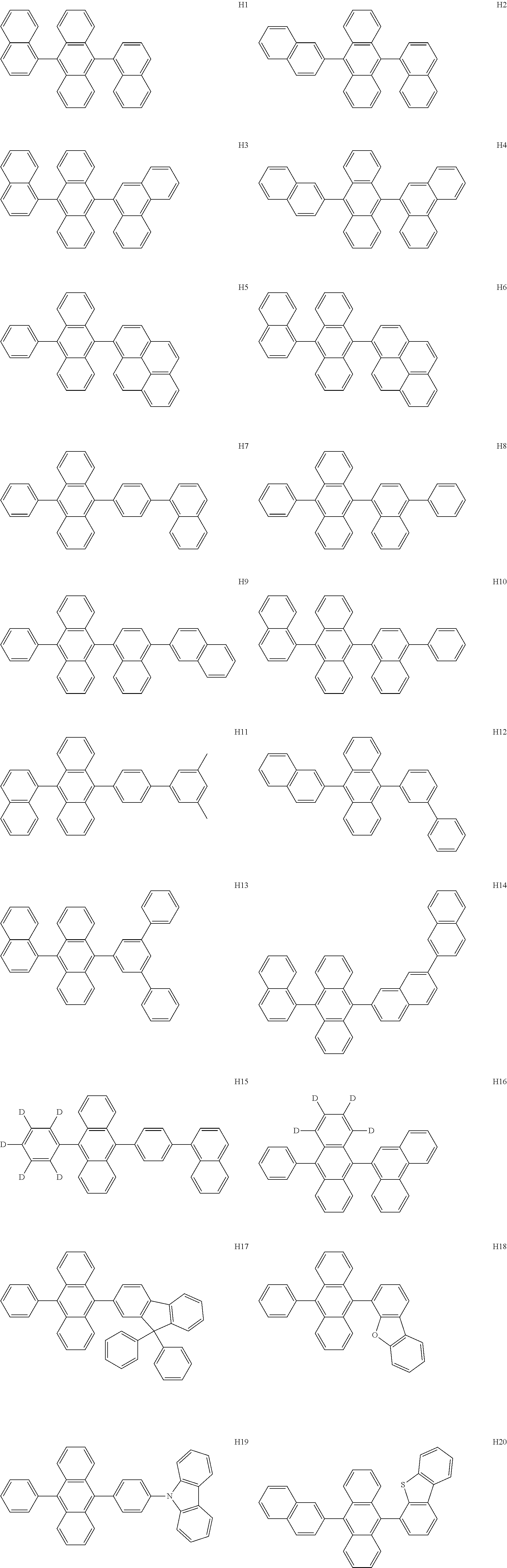

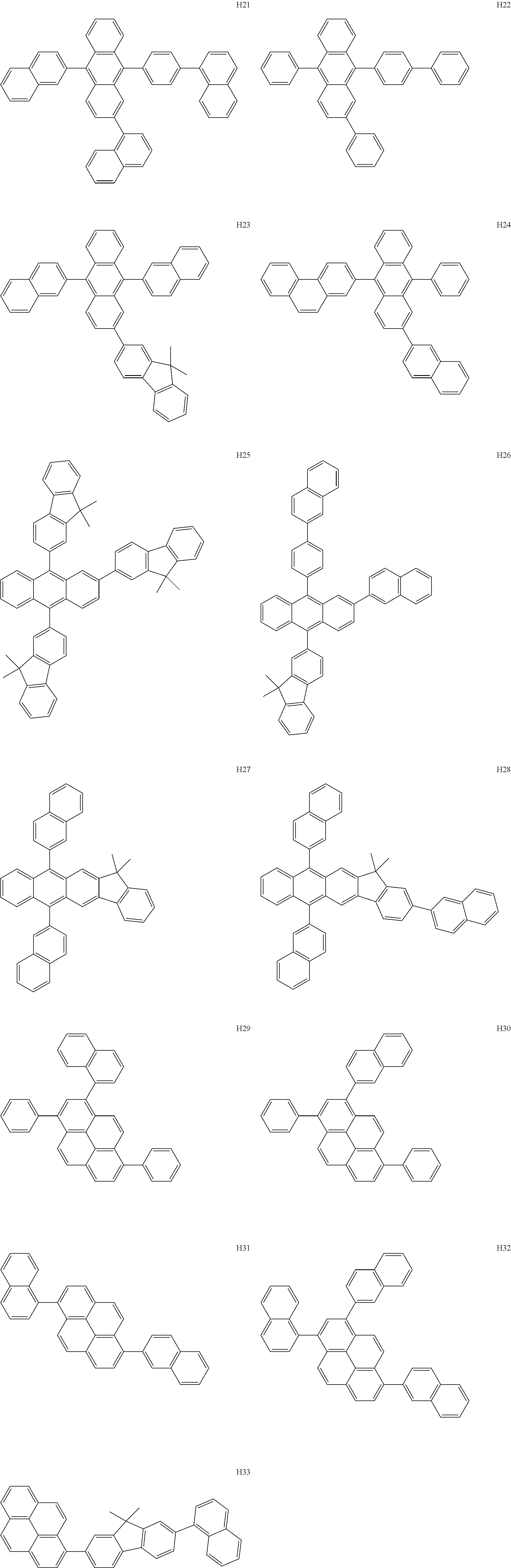

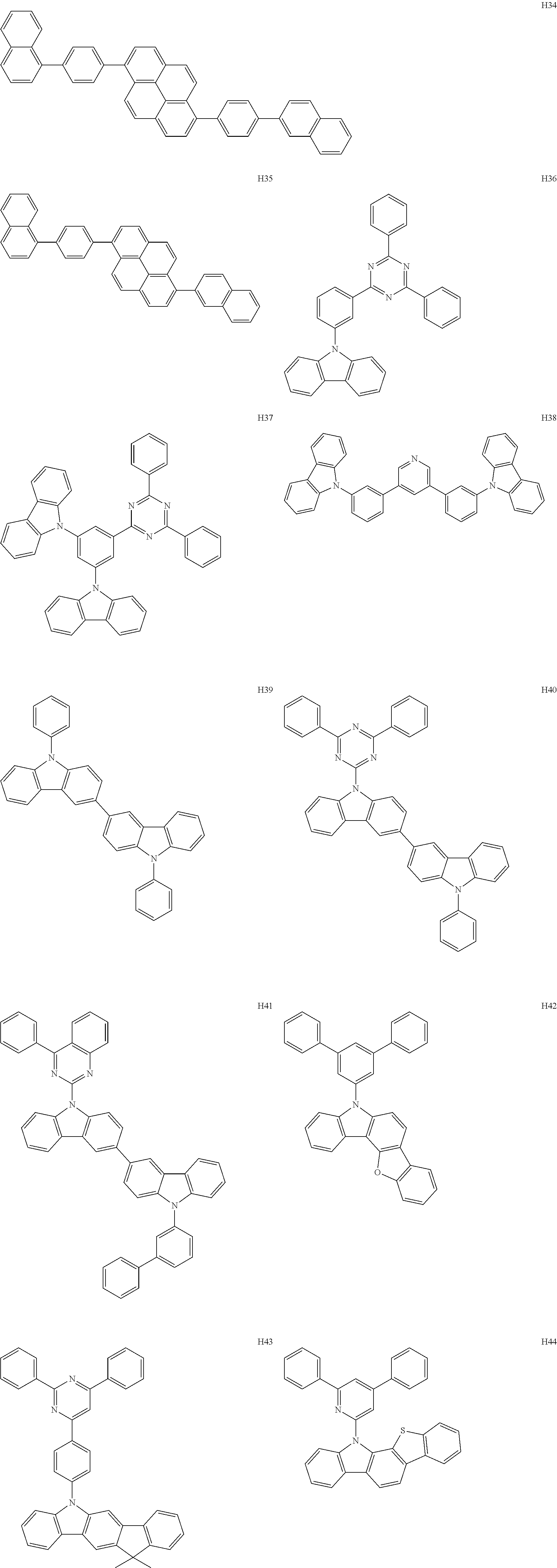

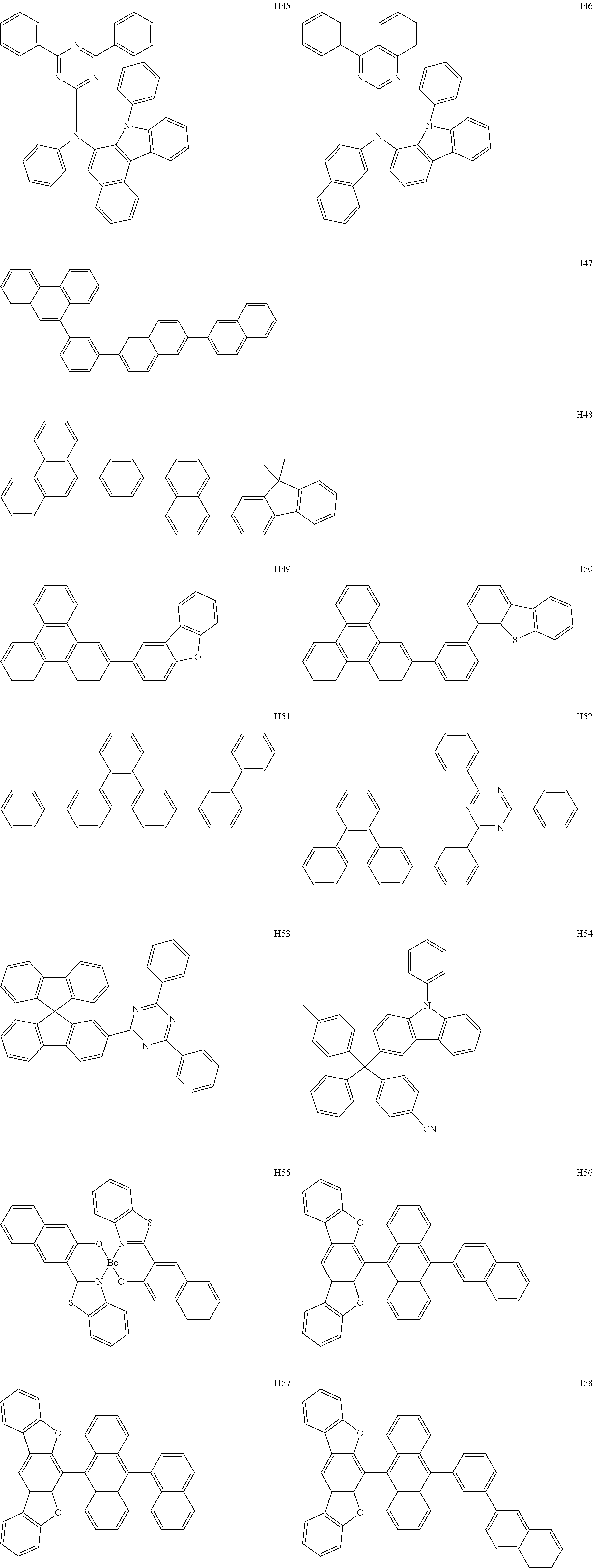

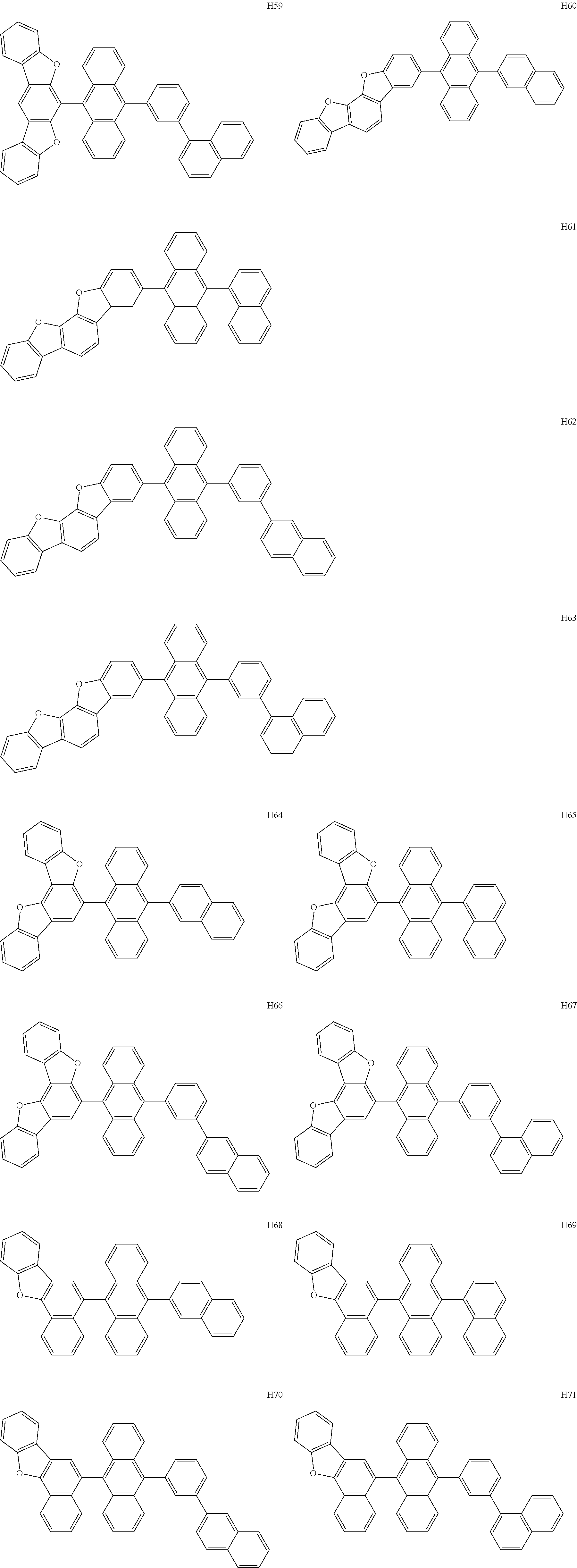

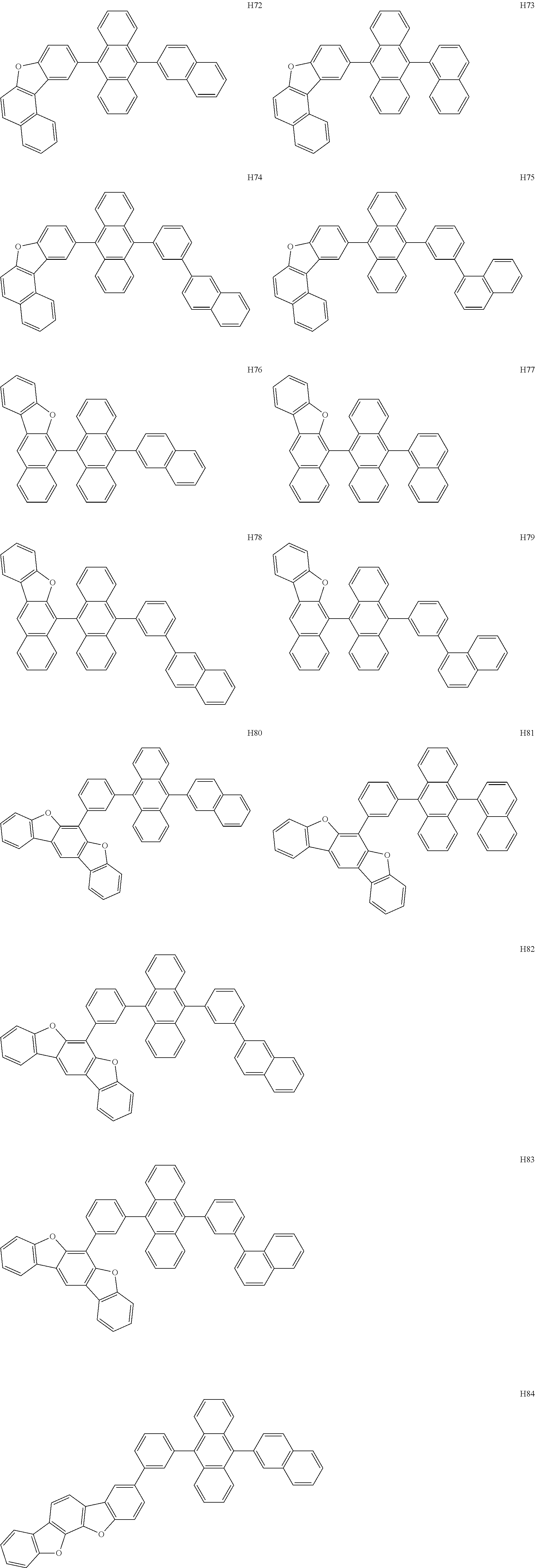

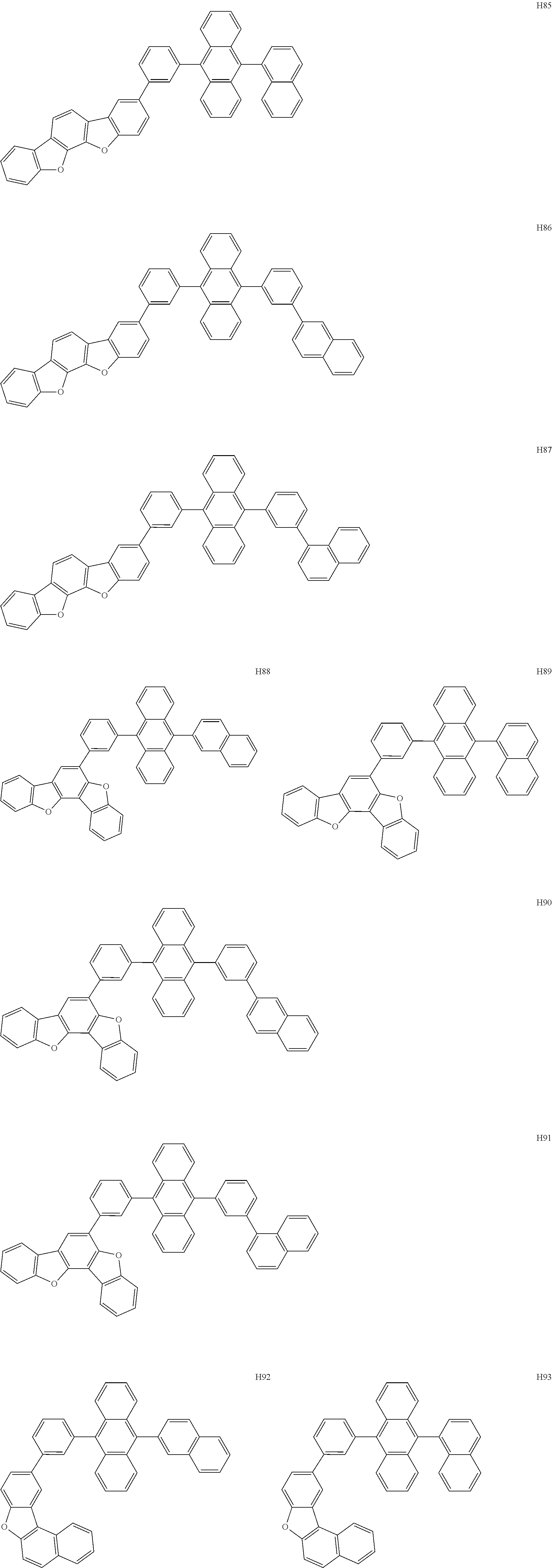

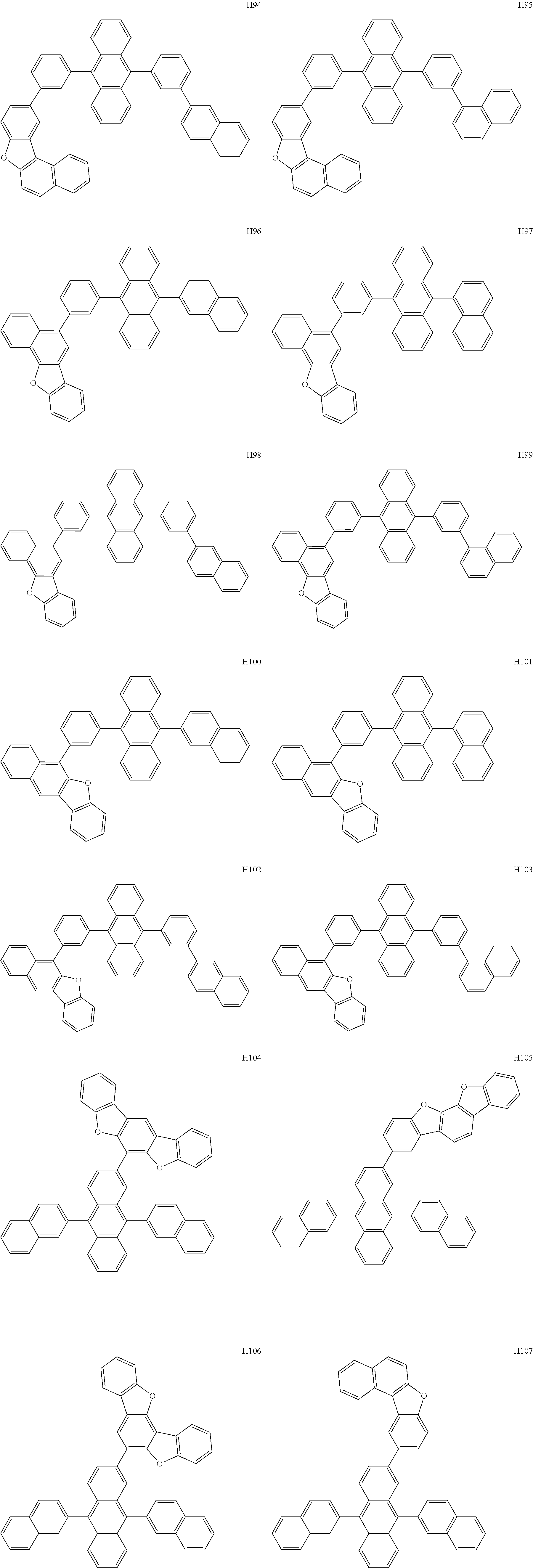

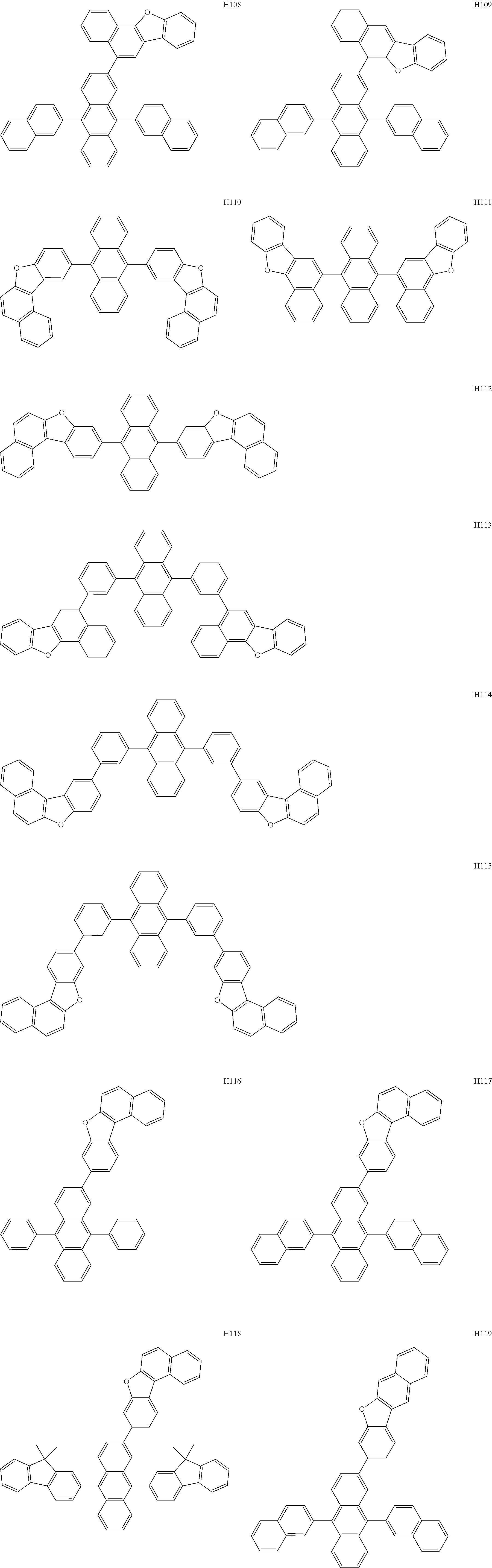

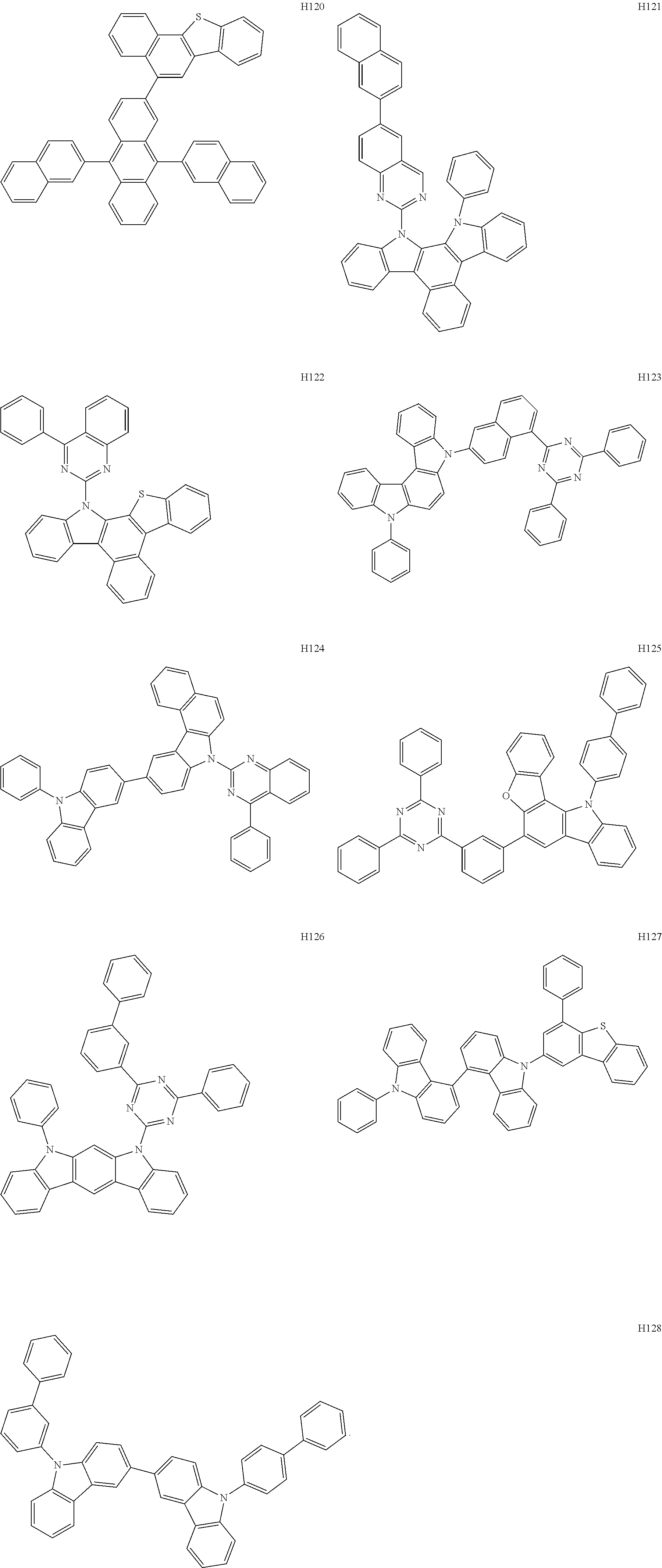

### Phosphorescent Dopant

In some embodiments, the phosphorescent dopant may include a central metal that includes at least one transition metal.

The phosphorescent dopant may include a monodentate ligand, a bidentate ligand, a tridentate ligand, a tetradentate ligand, a pentadentate ligand, a hexadentate ligand, or a combination thereof.

The phosphorescent dopant may be electrically neutral.

The phosphorescent dopant may include an organometallic compound represented by Formula 401:

M(L₄₀₁)_{xc1}(L₄₀₂)_{xc2} Formula 401

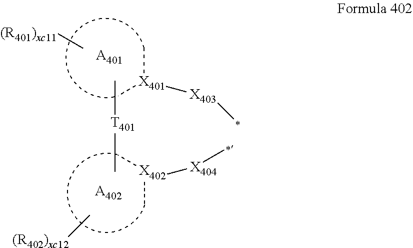

wherein, in Formulae 401 and 402,
M may be a transition metal (for example, iridium (Ir), platinum (Pt), palladium (Pd), osmium (Os), titanium (Ti), gold (Au), hafnium (Hf), europium (Eu), terbium (Tb), rhodium (Rh), rhenium (Re), or thulium (Tm)),
L₄₀₁ may be a ligand represented by Formula 402, and xc1 may be 1, 2, or 3, wherein when xc1 is 2 or 3, two or more of L₄₀₁(s) may be identical to or different from each other,
L₄₀₂ may be an organic ligand, and xc2 may be 0, 1, 2, 3, or 4, and when xc2 is 2, 3, or 4, two or more of L₄₀₂(s) may be identical to or different from each other,
X₄₀₁ and X₄₀₂ may each independently be nitrogen or carbon,
ring A₄₀₁ and ring A₄₀₂ may each independently be a C₃-C₆₀ carbocyclic group or a C₁-C₆₀ heterocyclic group,
T₄₀₁ may be a single bond, *—O—*′, *—S—*′, *—C(═O)—*′, *—N(Q₄₁₁)-*′, *—C(Q₄₁₁)(Q₄₁₂)-*′, *—C(Q₄₁₁)═C(Q₄₁₂)-*′, *—C(Q₄₁₁)=*′, or *═C(Q₄₁₁)=*′,
X₄₀₃ and X₄₀₄ may each independently be a chemical bond (for example, a covalent bond or a coordination bond), *—O—*′, *—S—*′, *—N(Q₄₁₃)-*′, *—B(Q₄₁₃)-*′, *—P(Q₄₁₃)-*′, *—C(Q₄₁₃)(Q₄₁₄)-*′, or *—Si(Q₄₁₃)(Q₄₁₄)-*′,
Q₄₁₁ to Q₄₁₄ may each be as described for Q₁ elsewhere herein,
R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, *—Si(Q₄₀₁)(Q₄₀₂)(Q₄₀₃)-*′, *—N(Q₄₀₁)(Q₄₀₂)-*′, *—B(Q₄₀₁)(Q₄₀₂)-*′, *—C(═O)(Q₄₀₁)-*′, *—S(═O)₂(Q₄₀₁)-*′, or *—P(═O)(Q₄₀₁)(Q₄₀₂)-*′,
Q₄₀₁ to Q₄₀₃ may each be as described for Q₁ elsewhere herein,
xc11 and xc12 may each independently be an integer from 0 to 10, and
“*” and “*” in Formula 402 each indicate a binding site to M in Formula 401 (i.e., a point of attachment).

For example, in Formula 402, i) X₄₀₁ may be nitrogen, and X₄₀₂ may be carbon, or ii) each of X₄₀₁ and X₄₀₂ may be nitrogen.

In some embodiments, when xc1 in Formula 402 is 2 or more, two ring A₄₀₁(s) in two or more of L₄₀₁(s) may be optionally linked to each other via T₄₀₂, which is a linking group, or two ring A₄₀₂(s) may be optionally linked to each other via T₄₀₃, which is a linking group (see Compounds PD1 to PD4 and PD7). T₄₀₂ and T₄₀₃ may each be as described for T₄₀₁.

L₄₀₂ in Formula 401 may be an organic ligand. For example, L₄₀₂ may include a halogen group, a diketone group (for example, an acetylacetonate group), a carboxylic acid group (for example, a picolinate group), —C(═O), an isonitrile group, —CN group, a phosphorus group (for example, a phosphine group, a phosphite group, etc.), or a combination thereof.

The phosphorescent dopant may include, for example, at least one of compounds PD1 to PD39, or a combination thereof:

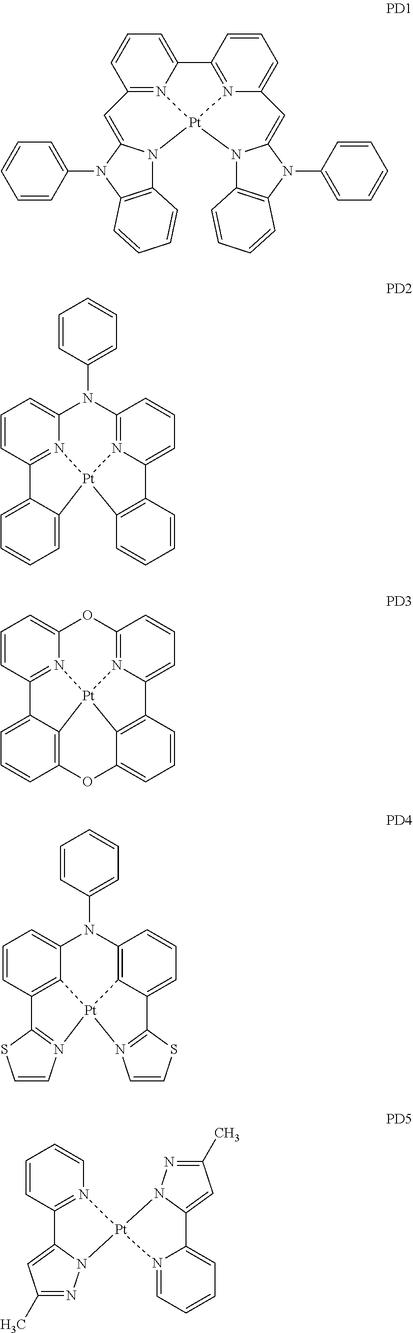

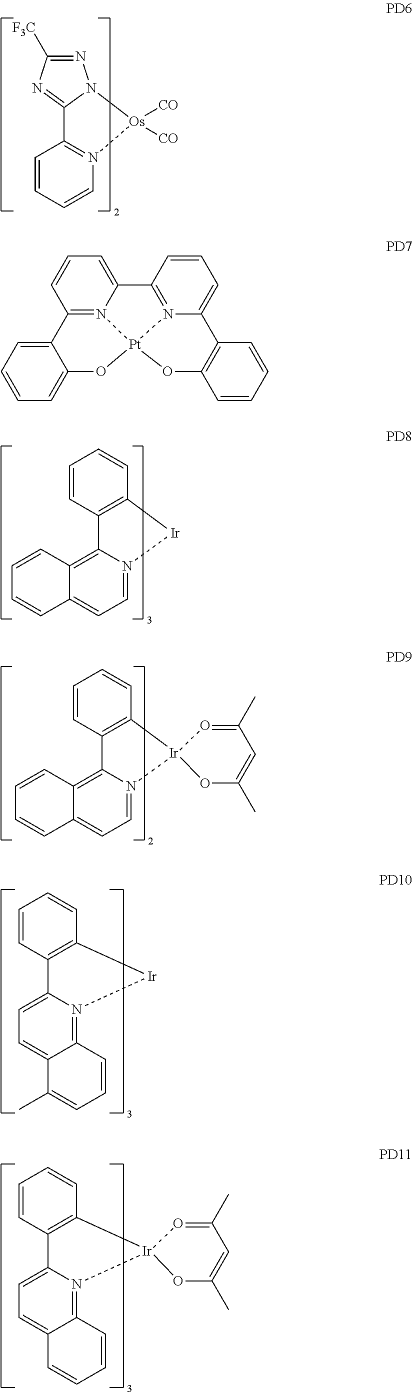

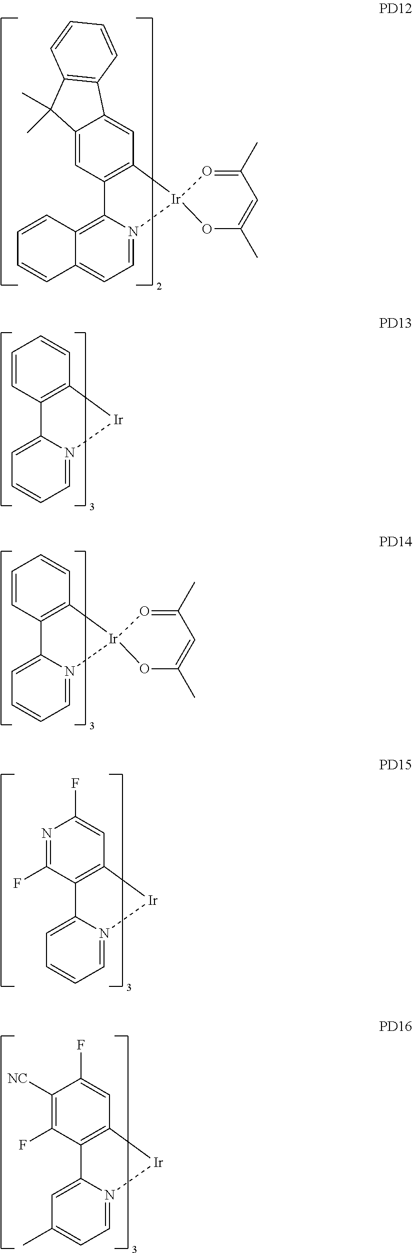

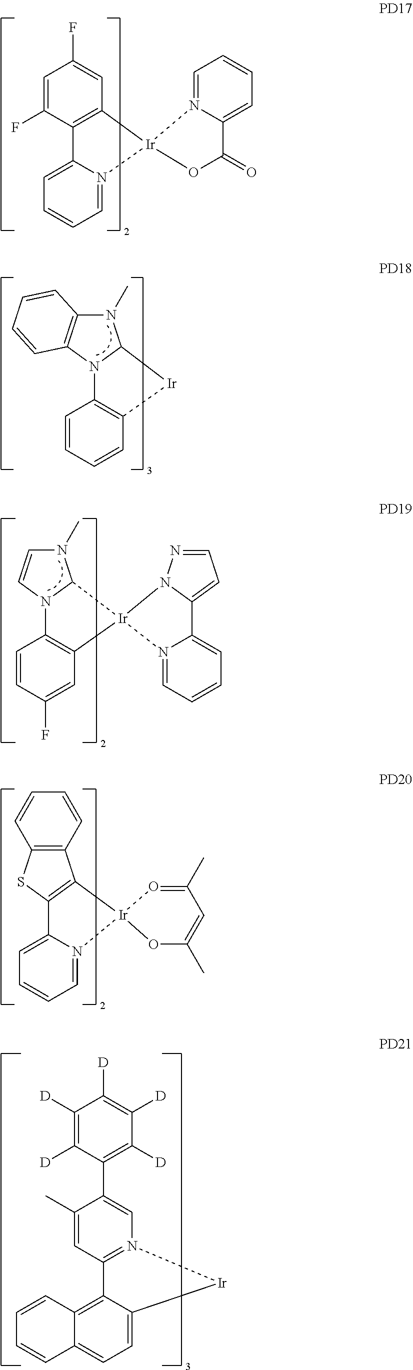

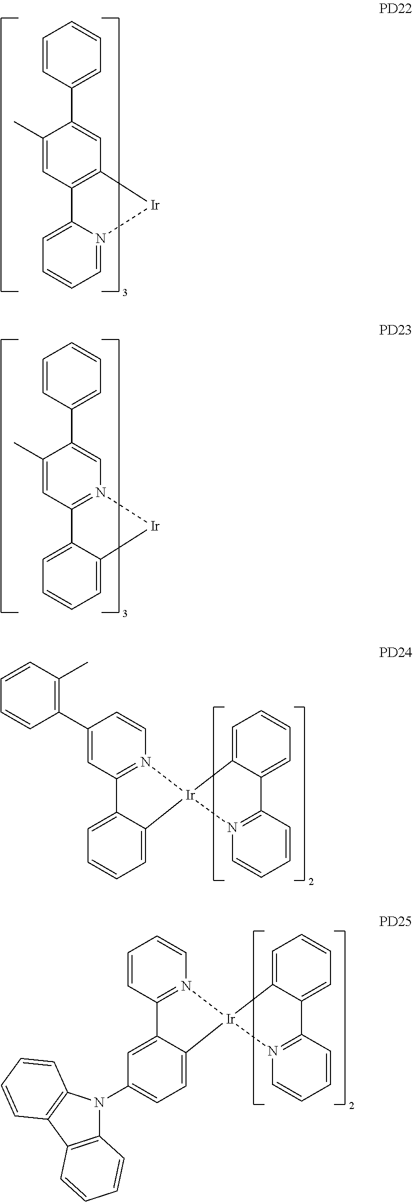

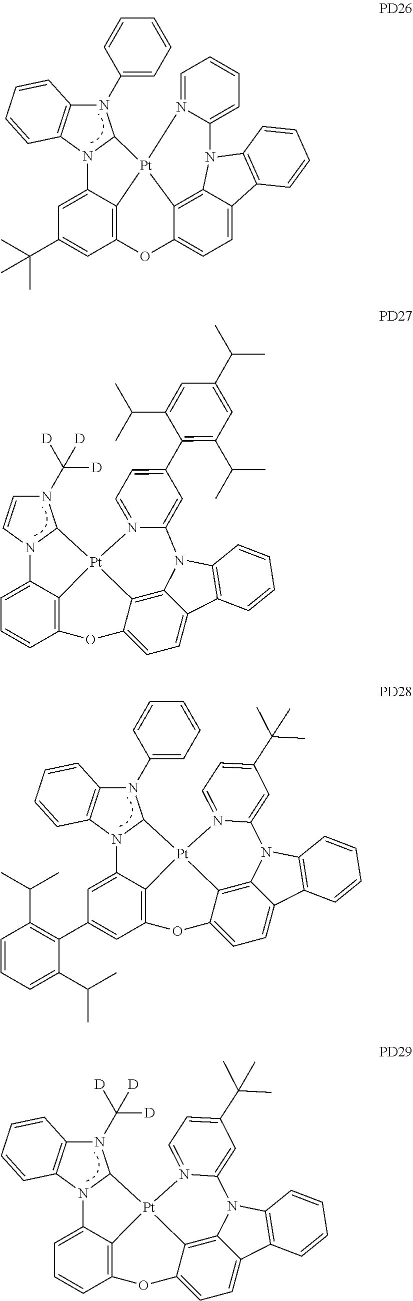

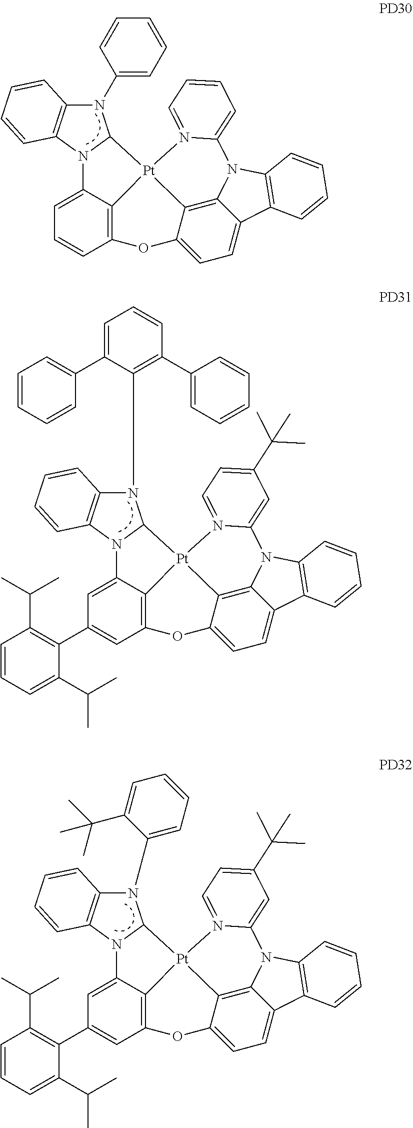

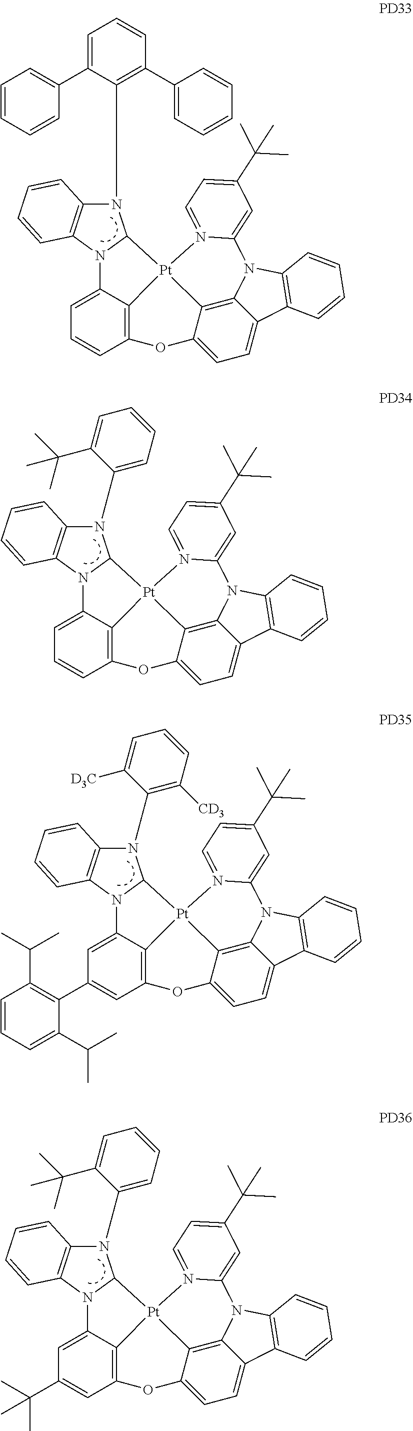

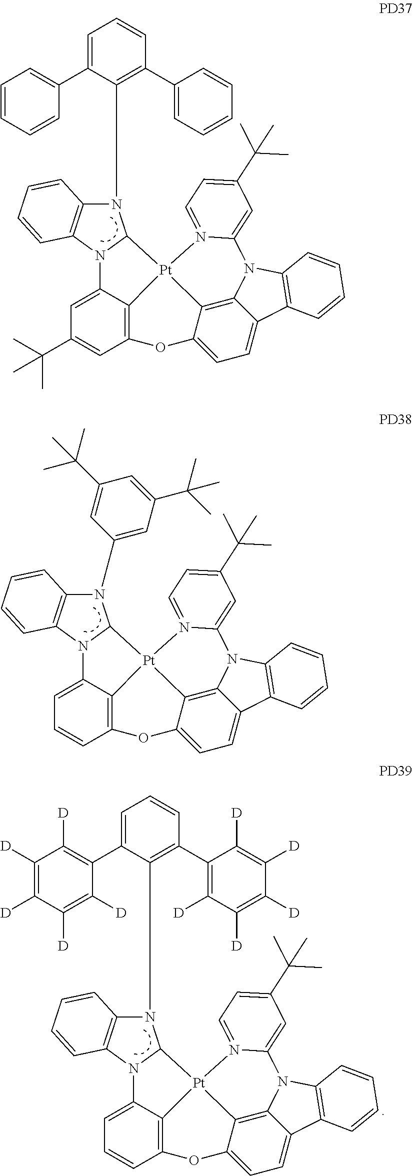

### Fluorescent Dopant

The fluorescent dopant may include an amine group-containing compound, a styryl group-containing compound, or a combination thereof. In some embodiments, the fluorescent dopant may include a compound represented by Formula 501:

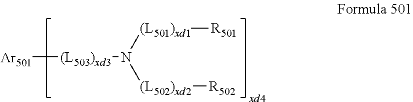

wherein, in Formula 501,
Ar₅₀₁, L₅₀₁ to L₅₀₃, R₅₀₁, and R₅₀₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xd1 to xd3 may each independently be 0, 1, 2, or 3, and
xd4 may be 1, 2, 3, 4, 5, or 6.

For example, Ar₅₀₁ in Formula 501 may be a condensed cyclic group (for example, an anthracene group, a chrysene group, or a pyrene group) in which three or more monocyclic groups are condensed together.

In some embodiments, xd4 in Formula 501 may be 2.

In an embodiment, the fluorescent dopant may include: at least one of Compounds FD1 to FD37; DPVBi; DPAVBi; or a combination thereof:

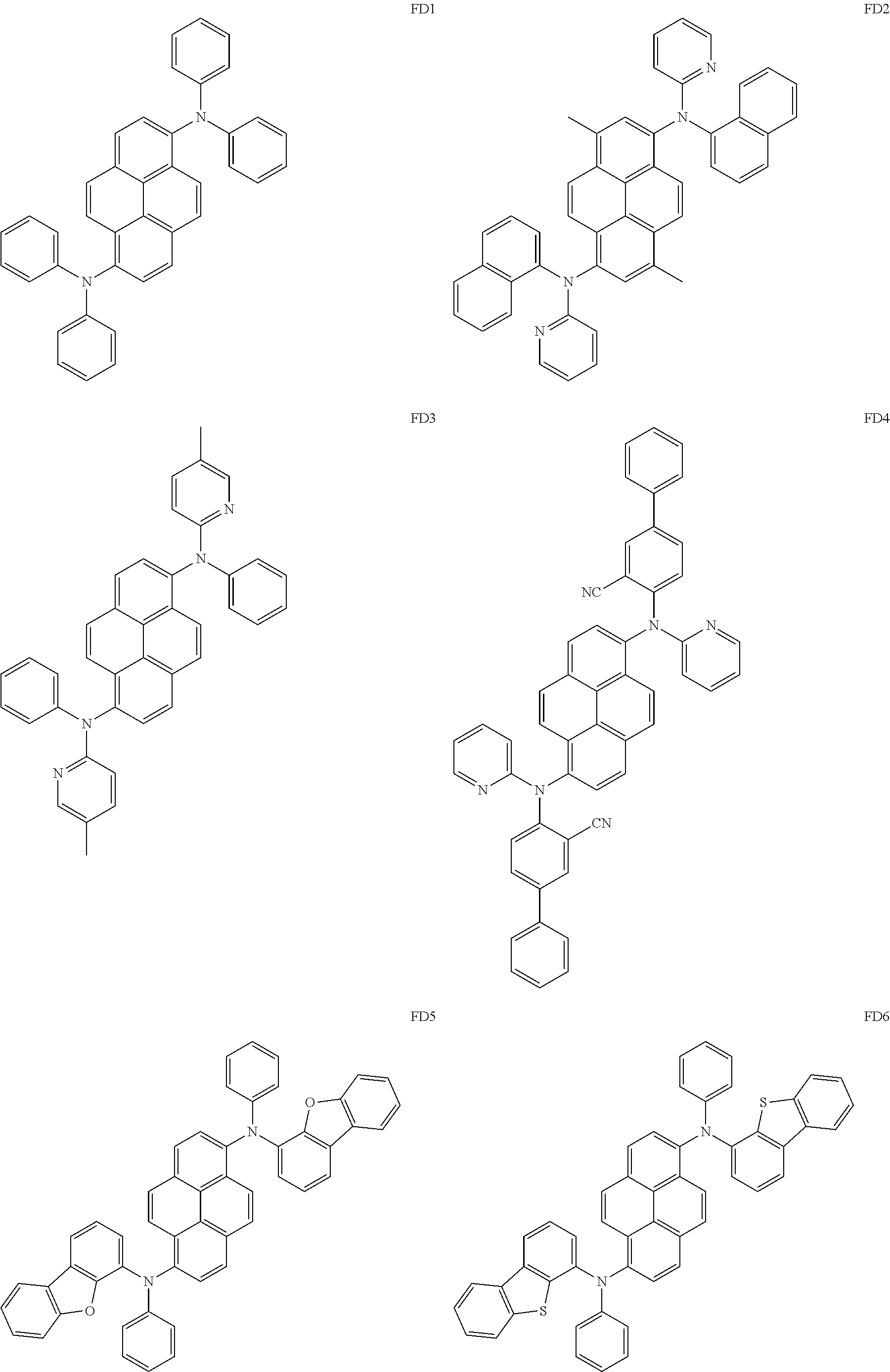

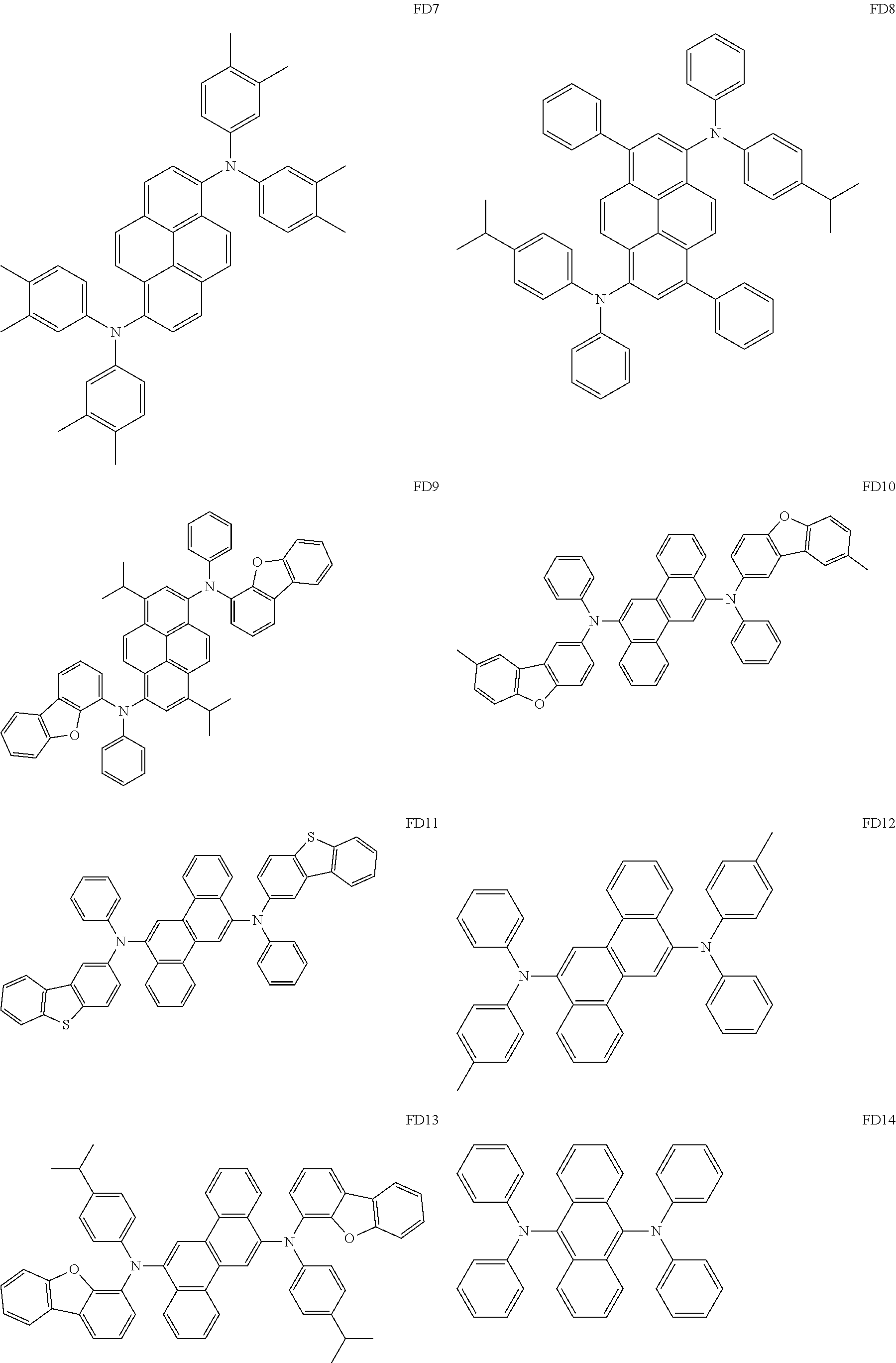

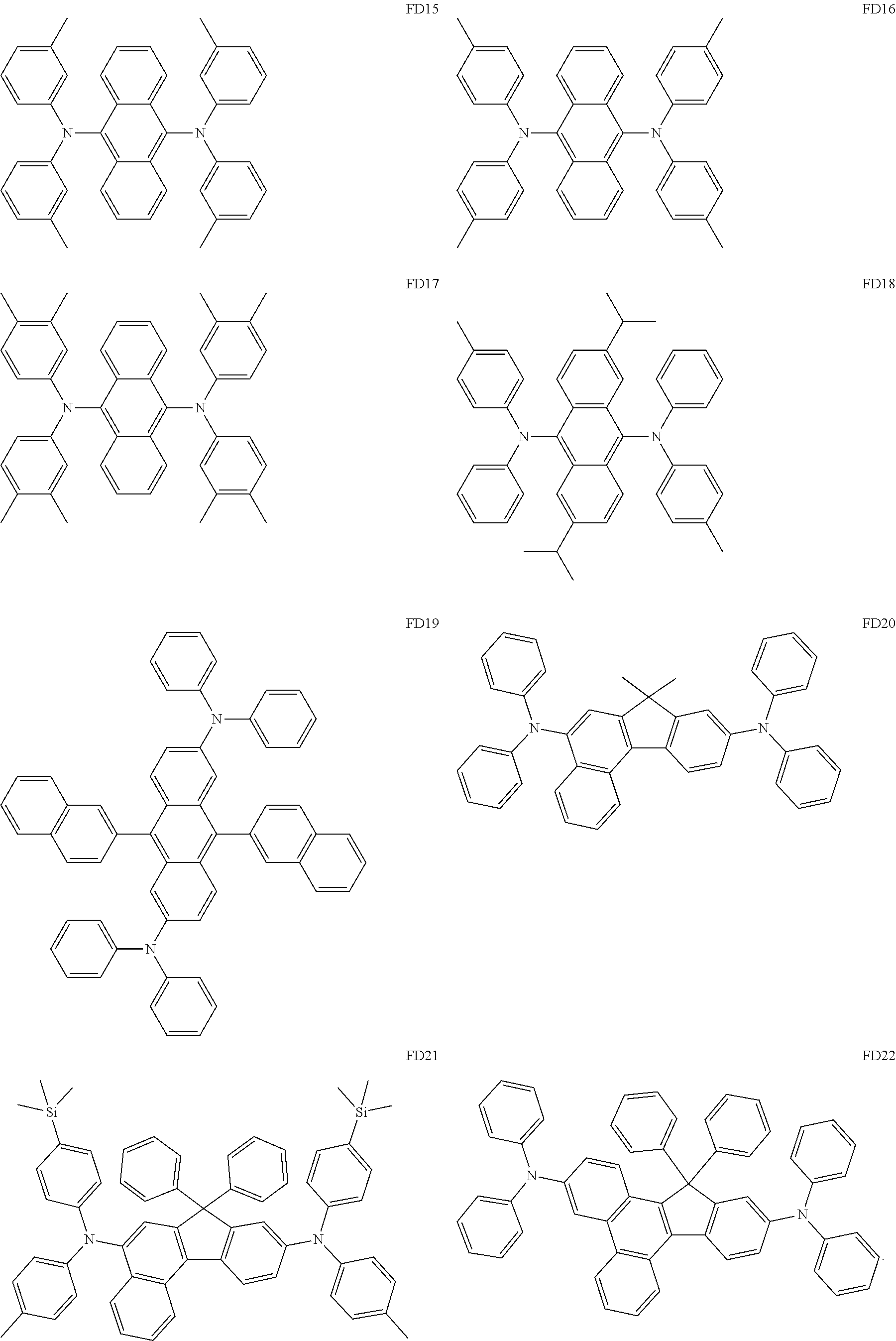

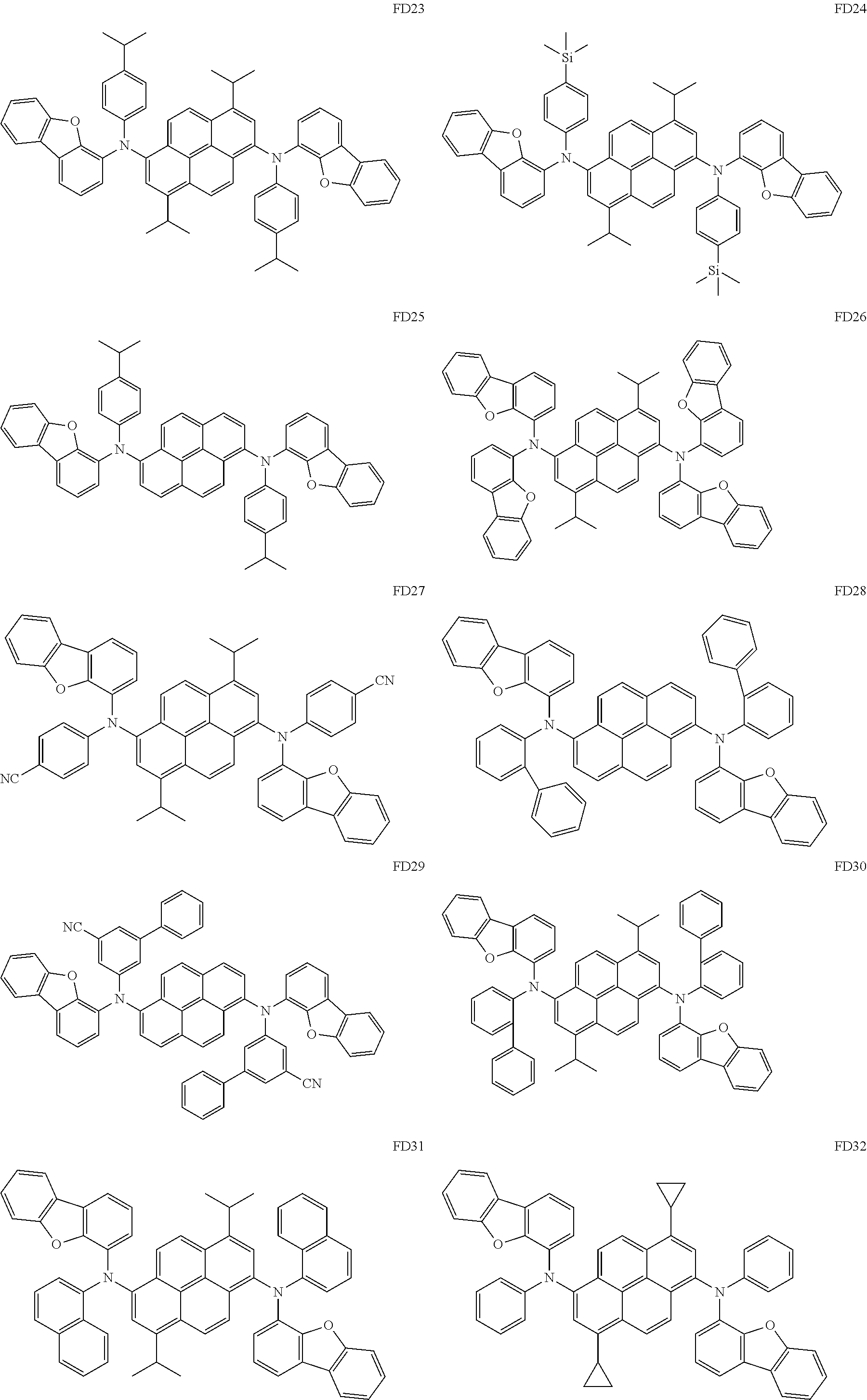

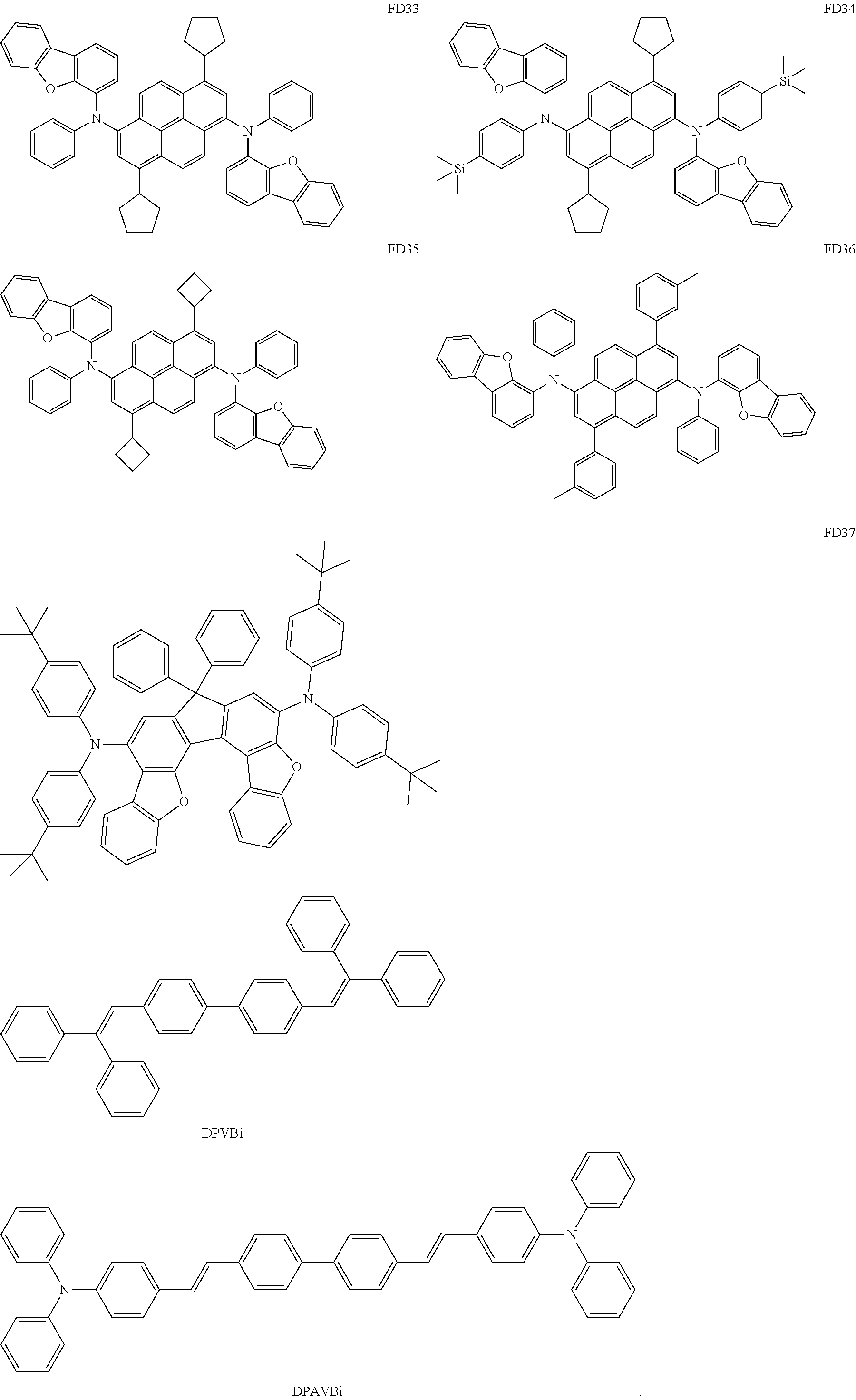

### Delayed Fluorescence Material

In an embodiment, the emission layer may include a delayed fluorescence material.

In the present specification, the delayed fluorescence material may be selected from compounds capable of emitting delayed fluorescent light based on a delayed fluorescence emission mechanism.

The delayed fluorescence material included in the emission layer may act as a host or a dopant depending on the type or kind of other materials included in the emission layer.

In some embodiments, the difference between the triplet energy level (eV) of the delayed fluorescence material and the singlet energy level (eV) of the delayed fluorescence material may be greater than or equal to 0 eV and less than or equal to 0.5 eV. When the difference between the triplet energy level (eV) of the delayed fluorescence material and the singlet energy level (eV) of the delayed fluorescence material satisfies the above-described range, up-conversion from the triplet state to the singlet state of the delayed fluorescence materials may effectively or suitably occur, and thus, the luminescence efficiency of the light-emitting device 10 may be enhanced or improved.

For example, the delayed fluorescence material may include i) a material including at least one electron donor (for example, a π electron-rich C₃-C₆₀ cyclic group, such as a carbazole group) and at least one electron acceptor (for example, a sulfoxide group, a cyano group, or a π electron-deficient nitrogen-containing C₁-C₆₀ cyclic group), and ii) a material including a C₈-C₆₀ polycyclic group in which two or more cyclic groups are condensed and simultaneously include boron (B).

Examples of the delayed fluorescence material may include at least one of Compounds DF1 to DF14:

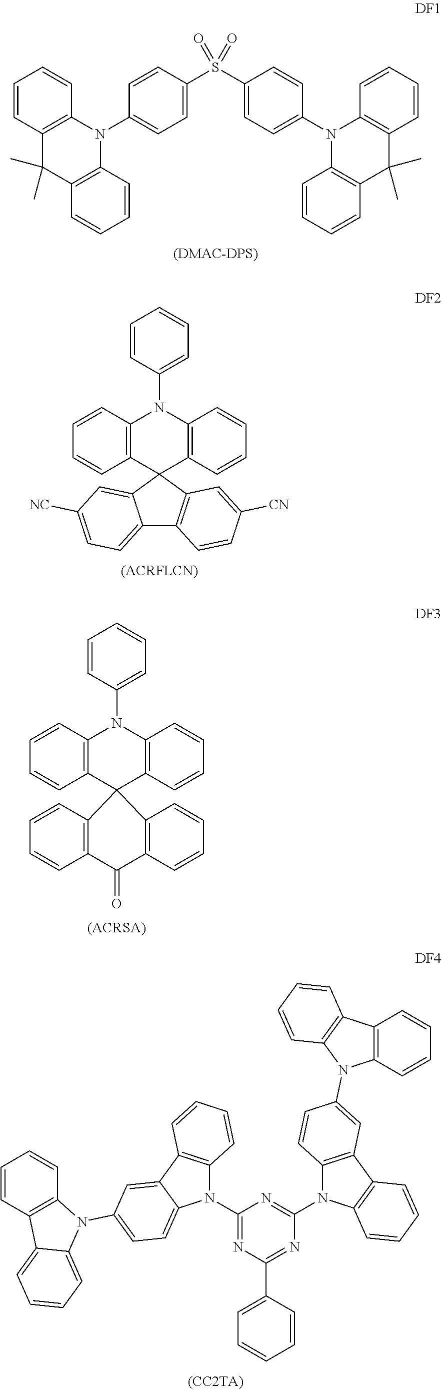

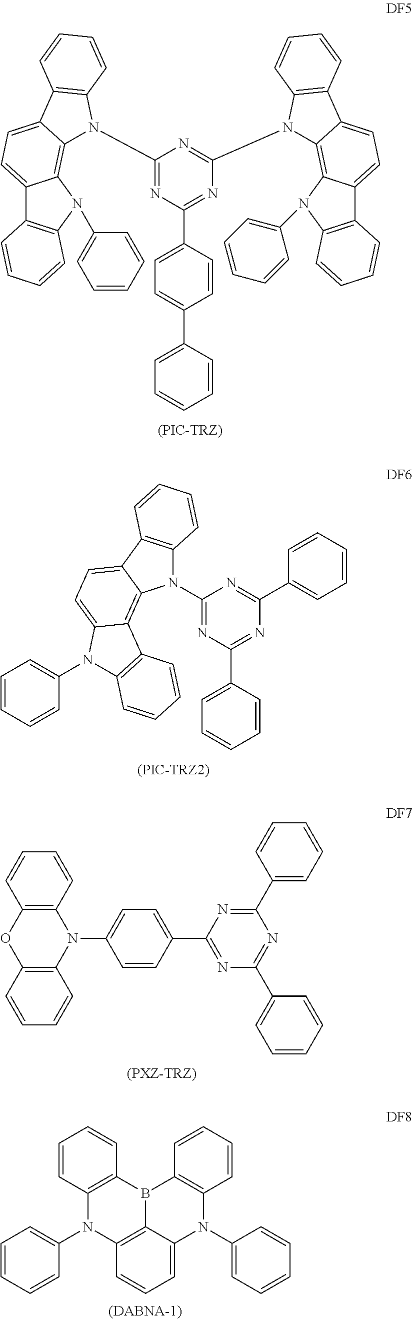

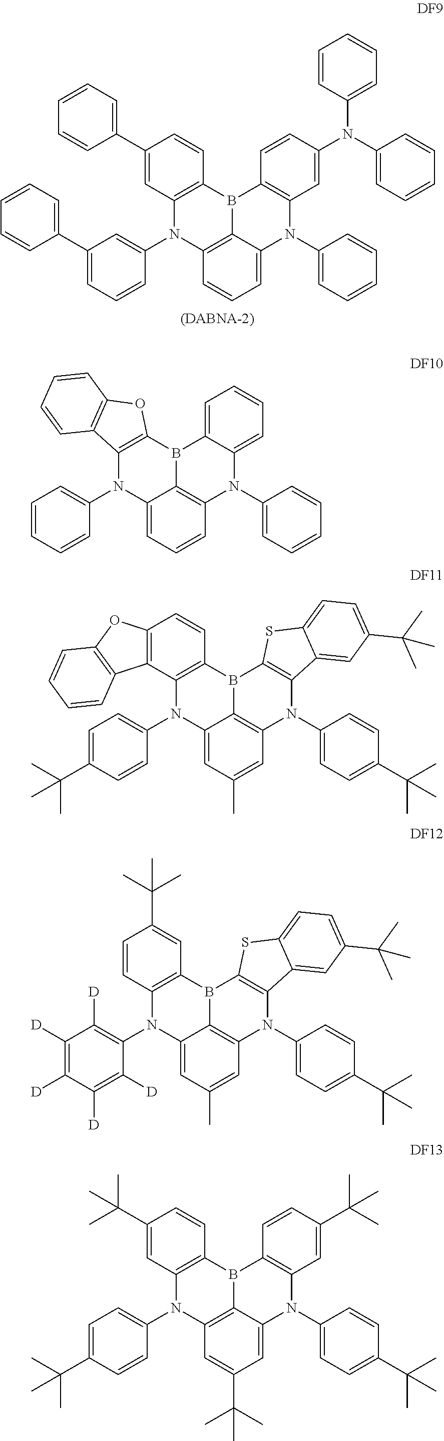

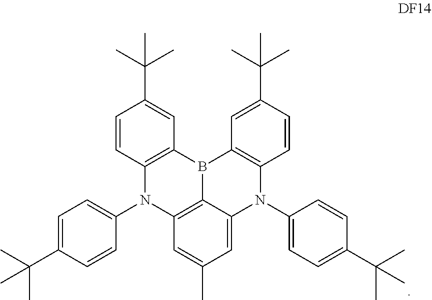 [SMILES: Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1]

### Quantum Dot

In an embodiment, the emission layer may include a quantum dot.

The term “quantum dot” as utilized herein refers to a crystal of a semiconductor compound. In an embodiment, the quantum dot may include any material configured to or capable of emitting light of one or more suitable emission wavelengths according to the size of the crystal.

In an embodiment, the quantum dot may have a diameter, for example, of about 1 nm to about 10 nm.

The quantum dot may be synthesized by a wet chemical process, a metal organic chemical vapor deposition process, a molecular beam epitaxy process, or any process similar thereto.

The wet chemical process is a method including mixing a precursor material with an organic solvent and then growing a quantum dot particle crystal. When the crystal grows, the organic solvent naturally acts as a dispersant coordinated on the surface of the quantum dot crystal and controls the growth of the crystal so that the growth of quantum dot particles can be controlled or selected. In an embodiment, the wet chemical process may be less expensive and/or easier to perform compared to vapor deposition methods, such as metal organic chemical vapor deposition (MOCVD) or molecular beam epitaxy (MBE).

The quantum dot may include Group II-VI semiconductor compounds, Group III-V semiconductor compounds, Group III-VI semiconductor compounds, Group I-III-VI semiconductor compounds, Group IV-VI semiconductor compounds, a Group IV element, a Group IV compound, or a combination thereof.

Non-limiting examples of the Group II-VI semiconductor compound are a binary compound, such as CdS, CdSe, CdTe, ZnS, ZnSe, ZnTe, ZnO, HgS, HgSe, HgTe, MgSe, MgS, and/or the like; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and/or the like; a quaternary compound, such as CdZnSeS, CdZnSeTe, CdZnSTe, CdHgSeS, CdHgSeTe, CdHgSTe, HgZnSeS, HgZnSeTe, HgZnSTe, and/or the like; or a combination thereof.

Non-limiting examples of the Group III-V semiconductor compound may include: a binary compound, such as GaN, GaP, GaAs, GaSb, AlN, AlP, AlAs, AlSb, InN, InP, InAs, InSb, and/or the like; a ternary compound, such as GaNP, GaNAs, GaNSb, GaPAs, GaPSb, AlNP, AlNAs, AlNSb, AlPAs, AlPSb, InGaP, InNP, InAlP, InNAs, InNSb, InPAs, InPSb, and/or the like; a quaternary compound, such as GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and/or the like; or a combination thereof. In some embodiments, the Group III-V semiconductor compound may further include a Group II element. Non-limiting examples of the Group III-V semiconductor compound further including a Group II element are InZnP, InGaZnP, InAlZnP, and/or the like.

Non-limiting examples of the Group III-VI semiconductor compound are: a binary compound, such as GaS, GaSe, Ga₂Se₃, GaTe, InS, InSe, In₂S₃, In₂Se₃, InTe, and/or the like; a ternary compound, such as InGaS₃, InGaSe₃, and/or the like; and a combination thereof.

Non-limiting examples of the Group I-III-VI semiconductor compound are: a ternary compound, such as AgInS, AgInS₂, CuInS, CuInS₂, CuGaO₂, AgGaO₂, AgAlO₂, and/or the like; or a combination thereof.

Non-limiting examples of the Group IV-VI semiconductor compound are: a binary compound, such as SnS, SnSe, SnTe, PbS, PbSe, PbTe, and/or the like; a ternary compound, such as SnSeS, SnSeTe, SnSTe, PbSeS, PbSeTe, PbSTe, SnPbS, SnPbSe, SnPbTe, and/or the like; a quaternary compound, such as SnPbSSe, SnPbSeTe, SnPbSTe, and/or the like; or a combination thereof.

The Group IV element or compound may include: a single element compound, such as Si, Ge, and/or the like; a binary compound, such as SiC, SiGe, and/or the like; or a combination thereof.

Each element included in a multi-element compound such as the binary compound, the ternary compound, and the quaternary compound may be present at a substantially uniform concentration or non-substantially uniform concentration in a particle.

In some embodiments, the quantum dot may have a single structure in which the concentration of each element in the quantum dot is substantially uniform. In some embodiments, the quantum dot may have a core-shell dual structure. For example, the material included in the core and the material included in the shell may be different from each other.

The shell of the quantum dot may act as a protective layer that prevents chemical degeneration of the core to maintain semiconductor characteristics, and/or as a charging layer that imparts electrophoretic characteristics to the quantum dot. The shell may be a single layer or a multi-layer. The interface between the core and the shell may have a concentration gradient in which the concentration of an element existing in the shell decreases toward the center of the core.

Non-limiting examples of the shell of the quantum dot may be an oxide of metal, metalloid, or non-metal, a semiconductor compound, and a combination thereof. Non-limiting examples of the oxide of metal, metalloid, or non-metal are a binary compound, such as SiO₂, Al₂O₃, TiO₂, ZnO, MnO, Mn₂O₃, Mn₃O₄, CuO, FeO, Fe₂O₃, Fe₃O₄, CoO, Co₃O₄, NiO, and/or the like; a ternary compound, such as MgAl₂O₄, CoFe₂O₄, NiFe₂O₄, CoMn₂O₄, and/or the like; and a combination thereof. Non-limiting examples of the semiconductor compound are, as described herein, a Group II-VI semiconductor compound; a Group III-V semiconductor compound; a Group III-VI semiconductor compound; a Group I-III-VI semiconductor compound; a Group IV-VI semiconductor compound; and a combination thereof. For example, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, and/or the like, or a combination thereof.

A full width at half maximum (FWHM) of the emission wavelength spectrum of the quantum dot may be about 45 nanometers (nm) or less, for example, about 40 nm or less, for example, about 30 nm or less, and within these ranges, color purity or color reproducibility may be increased. In some embodiments, because the light emitted through the quantum dot is emitted in all directions, the wide viewing angle may be enhanced or improved.

In some embodiments, the quantum dot may be in the form of a spherical particle, a pyramidal particle, a multi-arm particle, a cubic nanoparticle, a nanotube particle, a nanowire particle, a nanofiber particle, or a nanoplate particle.

Because the energy band gap may be adjusted by controlling the size of the quantum dot, light having one or more suitable wavelength bands may be obtained from the emission layer that includes the quantum dot. Accordingly, by utilizing quantum dots of different sizes, a light-emitting device that emits light of one or more suitable wavelengths may be implemented. In some embodiments, the size of the quantum dot may be selected to emit red, green and/or blue light. In some embodiments, the size of the quantum dot may be configured to emit white light by a combination of light of one or more suitable colors.

### Electron Transport Region in Interlayer 130

In some embodiments, the electron transport region may include: i) a single-layered structure that includes (e.g., consists of) a single layer including (e.g., consisting of) a single type or kind of material, ii) a single-layered structure that includes (e.g., consists of) a single layer that includes more than one type or kind of materials, or iii) a multi-layered structure including multiple layers that include more than one type or kind of materials.

In some embodiments, the electron transport region may include a buffer layer, a hole blocking layer, an electron control layer, an electron transport layer, an electron injection layer, and/or a combination thereof.

For example, the electron transport region may have an electron transport layer/electron injection layer structure, a hole blocking layer/electron transport layer/electron injection layer structure, an electron control layer/electron transport layer/electron injection layer structure, and/or a buffer layer/electron transport layer/electron injection layer structure. In some embodiments, the layers of each structure may be sequentially stacked starting from the emission layer.

In an embodiment, the electron transport region (for example, the buffer layer, the hole blocking layer, the electron control layer, or the electron transport layer in the electron transport region) may include a metal-free compound. In some embodiments, the metal-free compound includes at least one π electron-deficient nitrogen-containing C₁-C₆₀ cyclic group.

For example, the electron transport region may include a compound represented by Formula 601:

[Ar₆₀₁]ₓₑ₁₁-[(L₆₀₁)ₓₑ₁-R₆₀₁]ₓₑ₂₁ Formula 601
wherein, in Formula 601,
Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xe11 may be 1, 2, or 3,
xe1 may be 0, 1, 2, 3, 4, or 5,
R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),
Q₆₀₁ to Q₆₀₃ may each be as described for Q₁ elsewhere herein,
xe21 may be 1, 2, 3, 4, or 5,
at least one of Ar₆₀₁, L₆₀₁, and R₆₀₁ may each independently be air electron-deficient nitrogen-containing C₁-C₆₀ cyclic group unsubstituted or substituted with at least one R₁₀ₐ.

For example, when xe11 in Formula 601 is 2 or 3, two or more of Ar₆₀₁(s) may be linked to each other via a single bond.

In other embodiments, Ar₆₀₁ in Formula 601 may be a substituted or unsubstituted anthracene group.

In other embodiments, the electron transport region may include a compound represented by Formula 601-1:

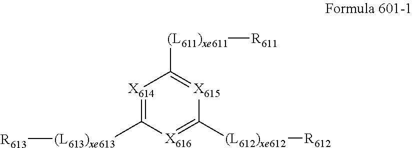

wherein, in Formula 601-1,
X₆₁₄ may be N or C(R₆₁₄), X₆₁₅ may be N or C(R₆₁₅), X₆₁₆ may be N or C(R₆₁₆), and at least one of X₆₁₄ to X₆₁₆ may be N,
L₆₁₁ to L₆₁₃ may each be as described for L₆₀₁ elsewhere herein,
xe611 to xe613 may each be as described for xe1 elsewhere herein,
R₆₁₁ to R₆₁₃ may each be as described for R₆₀₁ elsewhere herein, and
R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

For example, xe1 and xe611 to xe613 in Formulae 601 and 601-1 may each independently be 0, 1, or 2.

In some embodiments, the electron transport region may include at least one of Compounds ET1 to ET45, 2,9-dimethyl-4,7-diphenyl-1,10-phenanthroline (BCP), 4,7-diphenyl-1,10-phenanthroline (Bphen), Alq3, BAlq, TAZ, NTAZ, or a combination thereof:

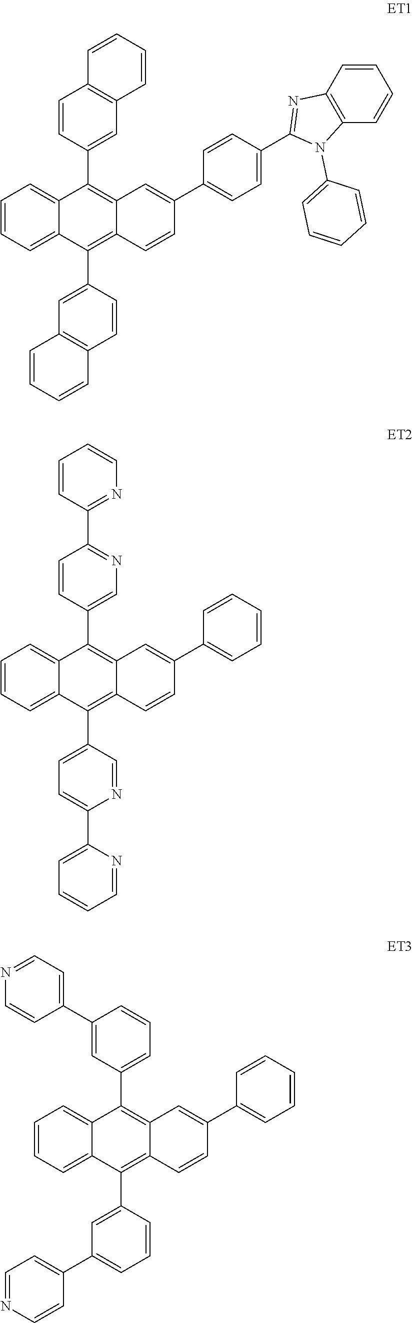 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

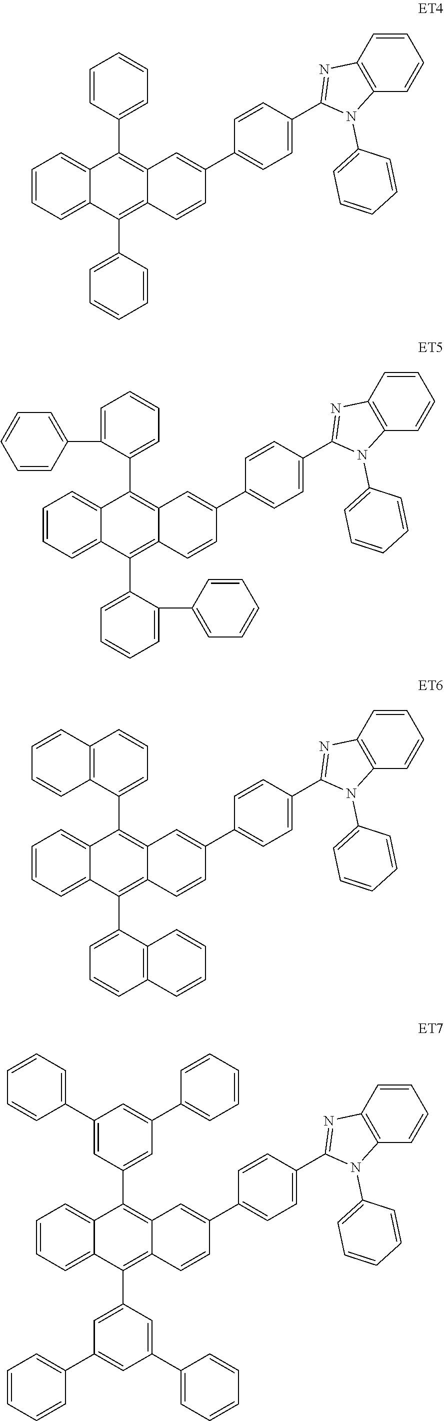 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

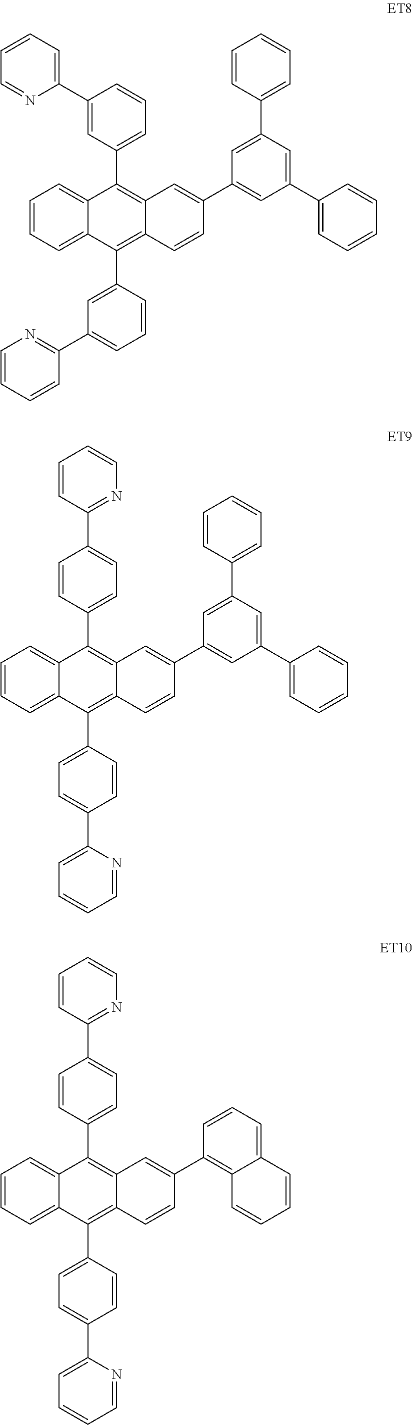

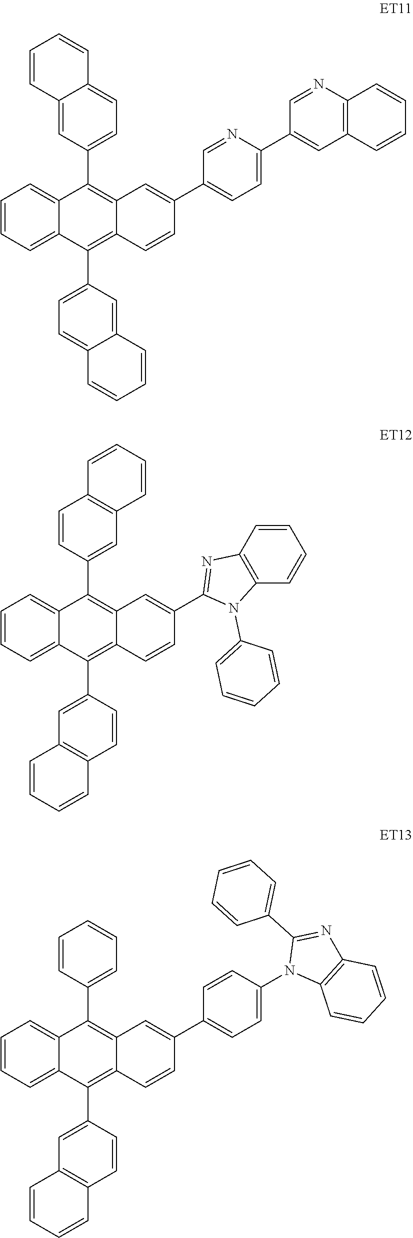

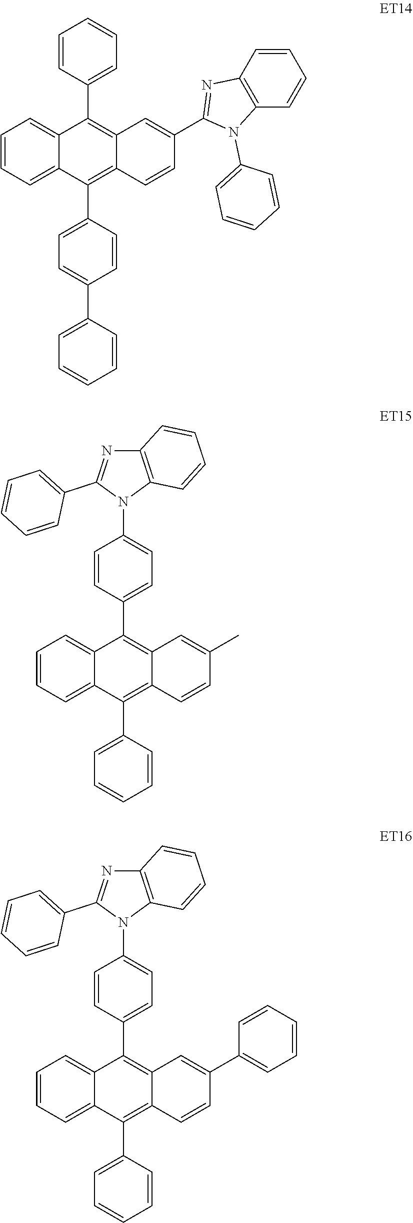 [SMILES: c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

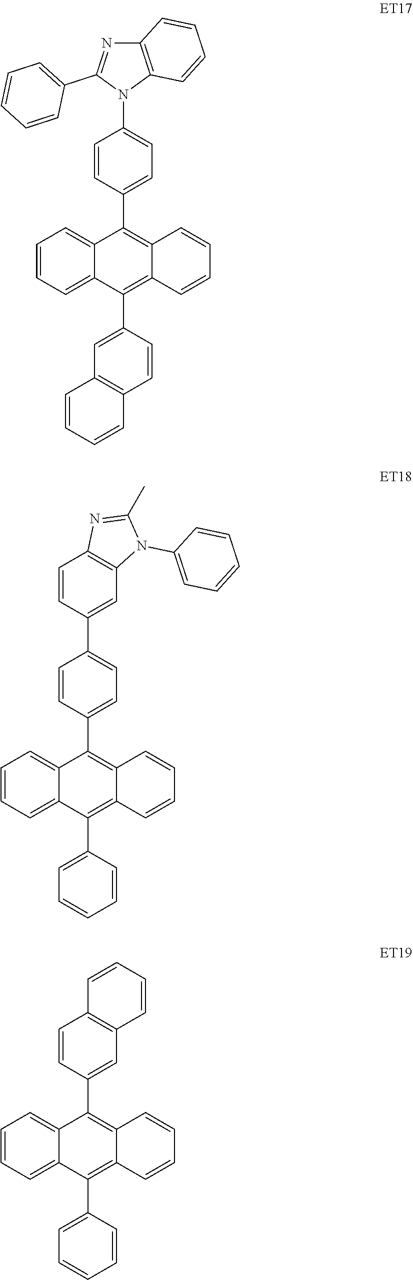

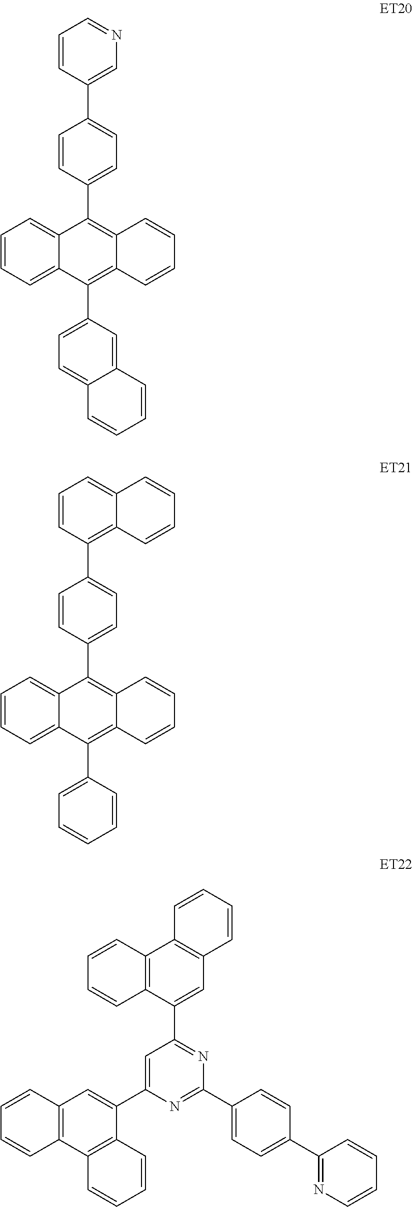

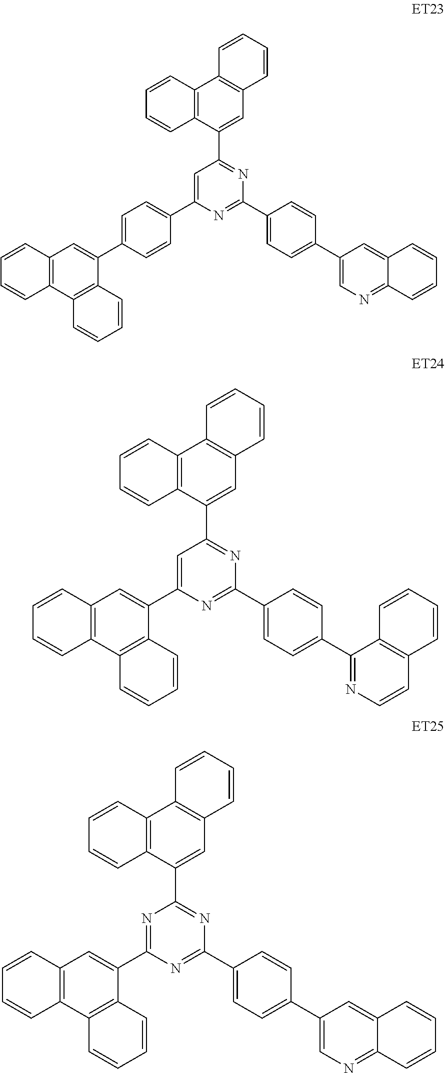 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1]

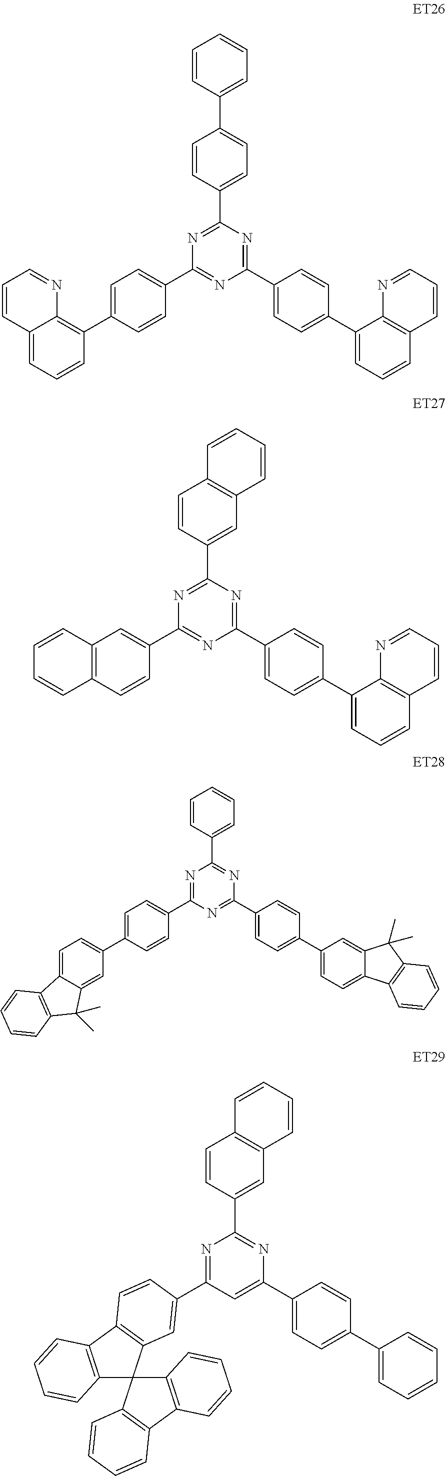 [SMILES: c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1]

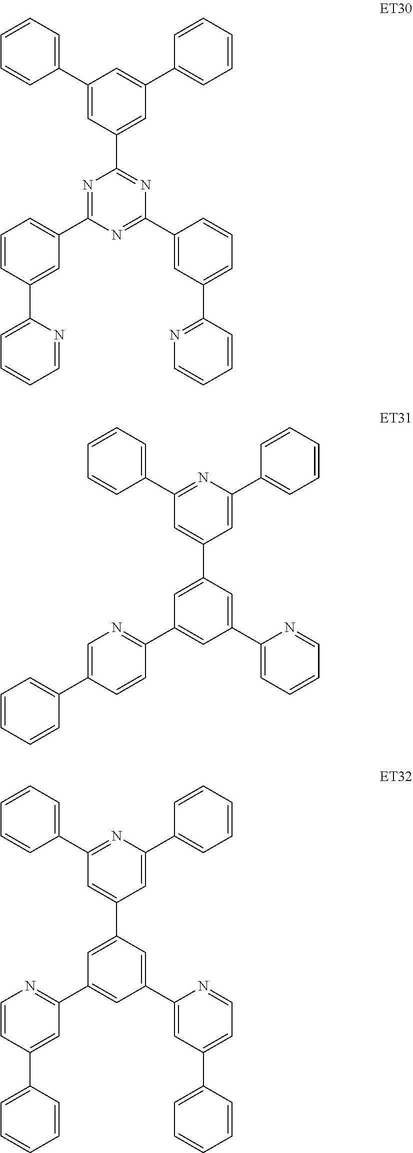

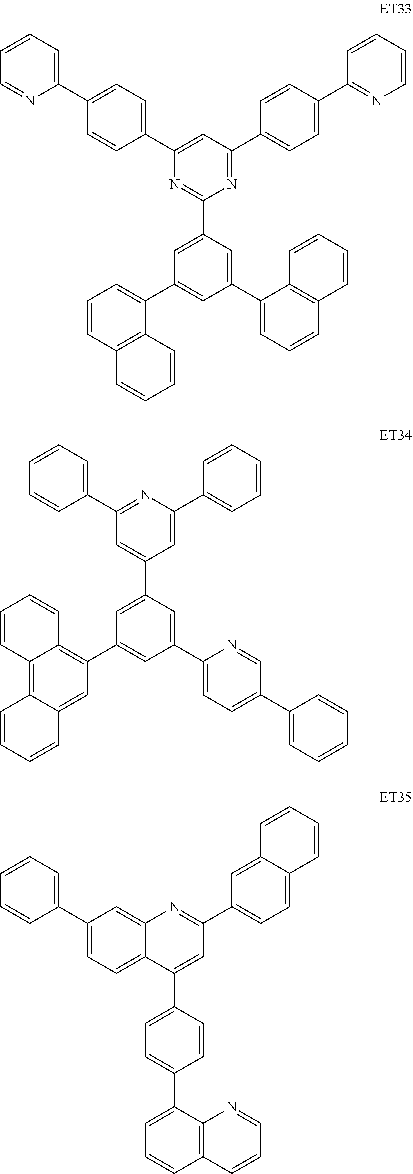 [SMILES: c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1]

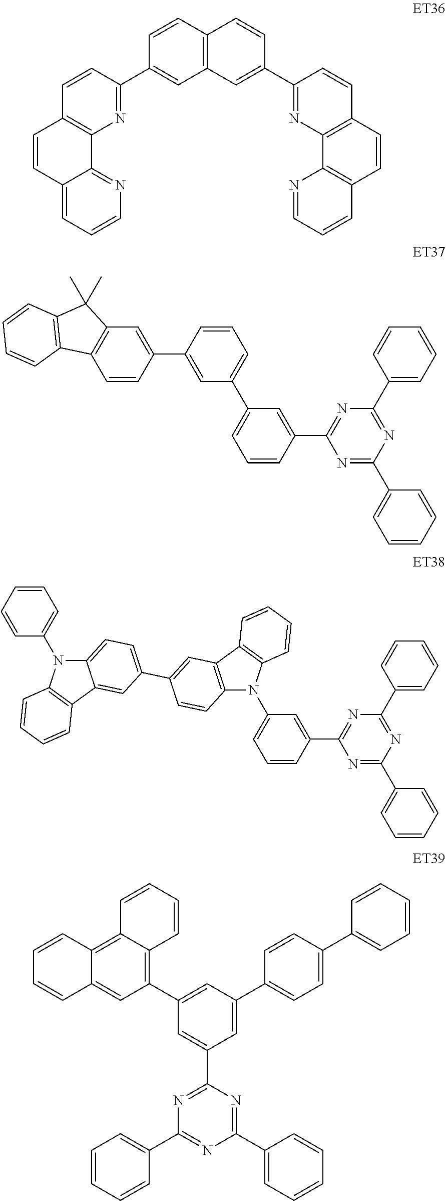

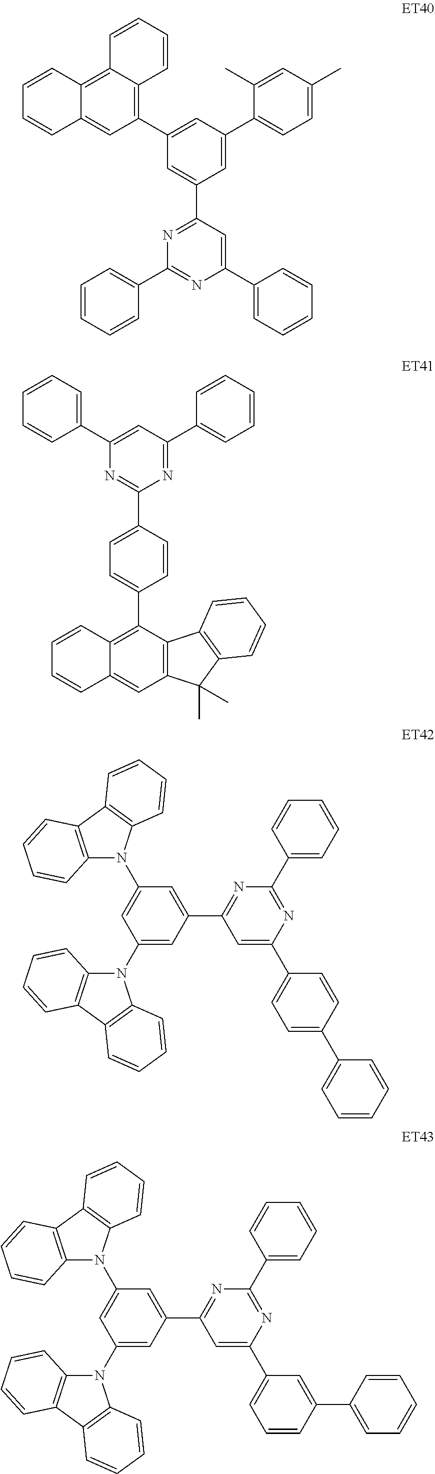 [SMILES: Cc1ccc(-c2cc(-c3cc(-c4ccccc4)nc(-c4ccccc4)n3)cc(-c3cc4ccccc4c4ccccc34)c2)c(C)c1 ; CC1(C)c2ccccc2-c2c1cc1ccccc1c2-c1ccc(-c2nc(-c3ccccc3)cc(-c3ccccc3)n2)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1]

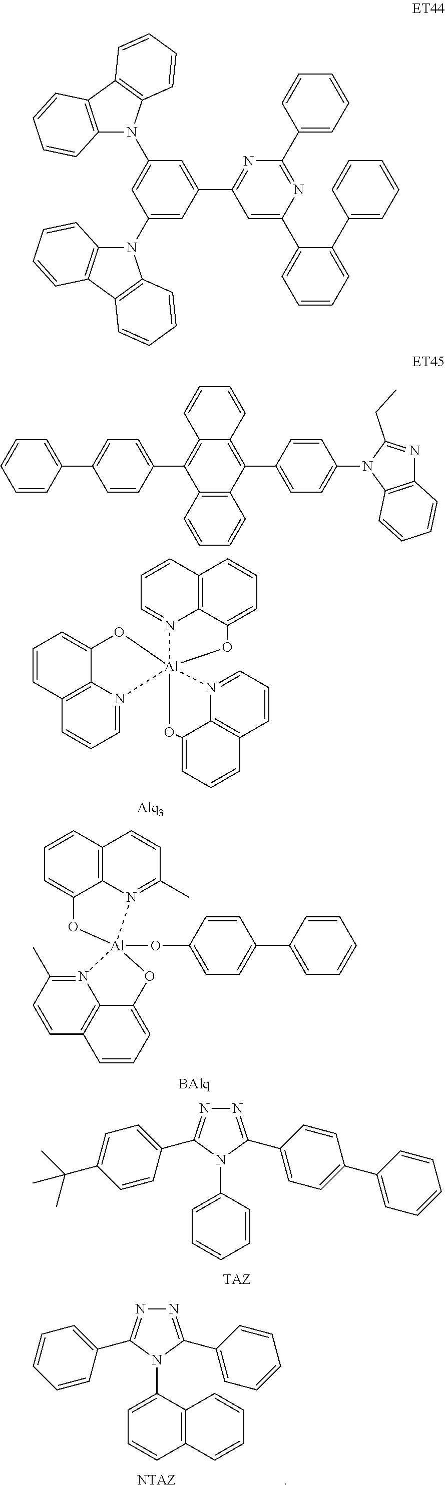

In an embodiment, the electron transport region includes a thickness. The thickness of the electron transport region may be about 100 Å to about 5,000 Å, for example, about 160 Å to about 4,000 Å.

In an embodiment, the electron transport region may include a buffer layer, a hole blocking layer, an electron control layer, an electron transport layer, and/or a combination thereof. In an embodiment, a thickness of the buffer layer, the hole blocking layer, and/or the electron control layer may each independently be about 20 Å to about 1000 Å, for example, about 30 Å to about 300 Å, and a thickness of the electron transport layer may be about 100 Å to about 1000 Å, for example, about 150 Å to about 500 Å. In an embodiment, thicknesses of the buffer layer, the hole blocking layer, the electron control layer, the electron transport layer, and/or the electron transport region within these ranges may be associated with a light-emitting device that has suitable or satisfactory electron transport characteristics without, or in the absence of, a substantial increase in driving voltage.

In an embodiment, the electron transport region (for example, the electron transport layer in the electron transport region) may include, in addition to the materials described above, a metal-containing material.

In an embodiment, the metal-containing material may include an alkali metal complex, an alkaline earth metal complex, or a combination thereof. The metal ion of an alkali metal complex may be a Li ion, a Na ion, a K ion, a Rb ion, or a Cs ion. The metal ion of an alkaline earth metal complex may be a Be ion, a Mg ion, a Ca ion, a Sr ion, or a Ba ion. A ligand coordinated with the metal ion of the alkali metal complex or the alkaline earth-metal complex may include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenylbenzimidazole, a hydroxyphenylbenzothiazole, a bipyridine, a phenanthroline, a cyclopentadiene, or a combination thereof.

For example, the metal-containing material may include a Li complex. The Li complex may include, for example, Compound ET-D1 (LiQ) or ET-D2:

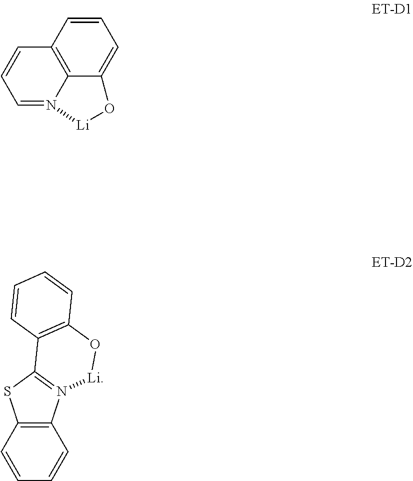

The electron transport region may include an electron injection layer. In an embodiment, the electron injection layer facilitates the injection of electrons from the second electrode 150. In an embodiment, the electron injection layer contacts the second electrode 150. In an embodiment, the electron injection layer may directly contact the second electrode 150.

In some embodiments, the electron injection layer may include: i) a single-layered structure that includes (e.g., consists of) a single layer including (e.g., consisting of) a single type or kind of material, ii) a single-layered structure that includes (e.g., consists of) a single layer that includes more than one type or kind of materials, or iii) a multi-layered structure including multiple layers that include more than one type or kind of materials.

In some embodiments, the electron injection layer may include an alkali metal, alkaline earth metal, a rare earth metal, an alkali metal-containing compound, alkaline earth metal-containing compound, a rare earth metal-containing compound, an alkali metal complex, an alkaline earth metal complex, a rare earth metal complex, or a combination thereof.

The alkali metal may include Li, Na, K, Rb, Cs, or a combination thereof. The alkaline earth metal may include Mg, Ca, Sr, Ba, or a combination thereof. The rare earth metal may include Sc, Y, Ce, Tb, Yb, Gd, or a combination thereof.

The alkali metal-containing compound, the alkaline earth metal-containing compound, and the rare earth metal-containing compound may be one or more of oxides, halides (for example, fluorides, chlorides, bromides, or iodides), or tellurides of the alkali metal, the alkaline earth metal, and/or the rare earth metal, or a combination thereof.

The alkali metal-containing compound may include: alkali metal oxides, such as Li₂O, Cs₂O, K₂O, and/or the like; alkali metal halides, such as LiF, NaF, CsF, KF, LiI, NaI, CsI, KI, and/or the like; or a combination thereof. The alkaline earth metal-containing compound may include an alkaline earth metal compound, such as BaO, SrO, CaO, BaₓSr₁₋ₓO (wherein x is a real number satisfying the condition of 0<x<1), BaₓCa₁₋ₓO (wherein x is a real number satisfying the condition of 0<x<1), and/or the like. The rare earth metal-containing compound may include YbF₃, ScF₃, Sc₂O₃, Y₂O₃, Ce₂O₃, GdF₃, TbF₃, YbI₃, ScI₃, TbI₃, and/or the like, or a combination thereof. In some embodiments, the rare earth metal-containing compound may include lanthanide metal telluride. Examples of the lanthanide metal telluride are LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, Lu₂Te₃, and/or the like.

The alkali metal complex, the alkaline earth-metal complex, and/or the rare earth metal complex may include i) one of ions of the alkali metal, the alkaline earth metal, and/or the rare earth metal and ii), a ligand bonded to the metal ion. For example, the ligand may be a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenyl benzimidazole, a hydroxyphenylbenzothiazole, a bipyridine, a phenanthroline, a cyclopentadiene, or a combination thereof.

In some embodiments, the electron injection layer may include (e.g., consist of): i) an alkali metal-containing compound (for example, an alkali metal halide); or ii) a) an alkali metal-containing compound (for example, an alkali metal halide), and b) an alkali metal, an alkaline earth metal, a rare earth metal, or a combination thereof. For example, the electron injection layer may include (e.g., be) a KI:Yb co-deposited layer, an RbI:Yb co-deposited layer, a LiF:Yb co-deposited layer, and/or the like.

In some embodiments, the electron injection layer may include an organic material (for example, a compound represented by Formula 601), in addition to an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or a combination thereof, as described herein. In some embodiments, the alkali metal, alkaline earth metal, rare earth metal, alkali metal-containing compound, alkaline earth metal-containing compound, rare earth metal-containing compound, alkali metal complex, alkaline earth-metal complex, rare earth metal complex, or a combination thereof may be uniformly or non-uniformly dispersed in a matrix including the organic material.

In an embodiment, the electron injection layer includes a thickness. The thickness of the electron injection layer may be about 1 Å to about 100 Å, and, for example, about 3 Å to about 90 Å. In an embodiment, the thickness of the electron injection layer within the ranges described may be associated with a light-emitting device that has suitable or satisfactory electron injection characteristics without, or in the absence of, a substantial increase in driving voltage.

### Second Electrode 150

Returning to FIG. 1, the second electrode 150 may be located at (e.g., on), the interlayer 130 having a structure as described herein. The second electrode 150 may be a cathode, i.e., an electron injection electrode. In an embodiment, the material for the second electrode 150 may include a metal, an alloy, an electrically conductive compound, or a combination thereof. In an embodiment, the material for the second electrode 150 has a low work-function. The term “low work-function material” as utilized herein refers to a substance (e.g., a metal or metal alloy) that requires a relatively low amount of energy to emit electrons from its surface.

The second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or a combination thereof. The second electrode 150 may be a transmissive electrode, a semi-transmissive electrode, or a reflective electrode.

The second electrode 150 may have a single-layered structure or a multi-layered structure including a plurality of layers.

### Capping Layer

In an embodiment, a first capping layer may be external to, or located outside, the first electrode 110, and/or a second capping layer may be external to, or located outside, the second electrode 150. In an embodiment, the light-emitting device 10 may have a structure in which the first capping layer, the first electrode 110, the interlayer 130, and the second electrode 150 are sequentially stacked in the stated order. In an embodiment, the first electrode 110, the interlayer 130, the second electrode 150, and the second capping layer are sequentially stacked in the stated order. In an embodiment, the first capping layer, the first electrode 110, the interlayer 130, the second electrode 150, and the second capping layer are sequentially stacked in the stated order.

Light generated in the emission layer of the interlayer 130 may be directed away from the light-emitting device 10 through the first electrode 110, (which is a semi-transmissive electrode or a transmissive electrode), and the first capping layer. Light generated in the emission layer of the interlayer 130 may be directed away from the light-emitting device 10 through the second electrode 150, (which is a semi-transmissive electrode or a transmissive electrode), and the second capping layer.

The first capping layer and the second capping layer may be configured to enhance or increase external emission efficiency according to the principle of constructive interference. Accordingly, the light extraction efficiency of the light-emitting device 10 is enhanced or increased, so that the luminescence efficiency of the light-emitting device 10 may be enhanced or improved.

Each of the first capping layer and the second capping layer may include a material having a refractive index of 1.6 or more (at 589 nm).

The first capping layer and the second capping layer may each independently be an organic capping layer including an organic material, an inorganic capping layer including an inorganic material, or an organic-inorganic composite capping layer including an organic material and an inorganic material.

At least one of the first capping layer or the second capping layer may each independently include a carbocyclic compound, a heterocyclic compound, an amine group-containing compound, a porphine derivative, a phthalocyanine derivative, a naphthalocyanine derivative, an alkali metal complex, an alkaline earth metal complex, or a combination thereof. In an embodiment, the carbocyclic compound, the heterocyclic compound, and the amine group-containing compound may be substituted with O, N, S, Se, Si, F, Cl, Br, I, or a combination thereof. In some embodiments, at least one of the first capping layer and the second capping layer may each independently include an amine group-containing compound.

For example, at least one of the first capping layer or the second capping layer may independently include a compound represented by Formula 201, a compound represented by Formula 202, or a combination thereof.

In some embodiments, at least one of the first capping layer or the second capping layer may independently include at least one of Compounds HT28 to HT33, at least one of Compounds CP1 to CP6, β-NPB, or a combination thereof:

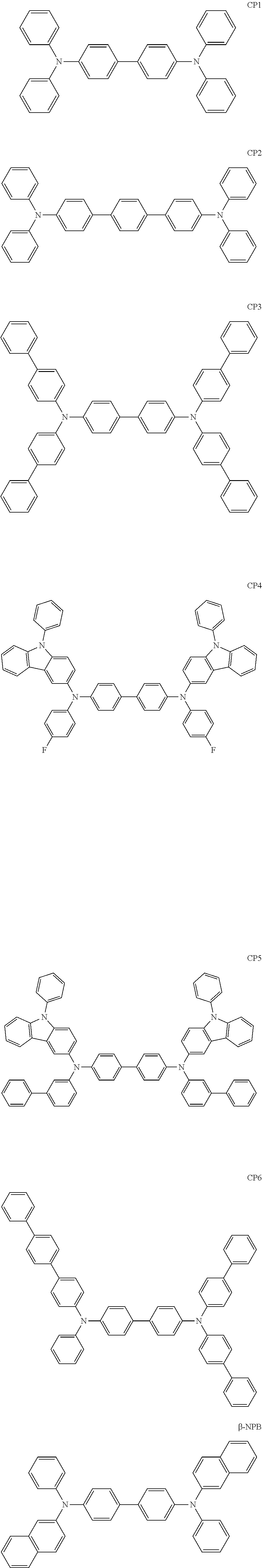

### Film

The amine-containing compound represented by Formula 1 may be included in one or more suitable films. Accordingly, another aspect provides a film including the amine-containing compound represented by Formula 1. The film may be, for example, an optical member or a light control component, (for example, a color filter, a color conversion member, a capping layer, a light extraction efficiency enhancement layer, a selective light absorbing layer, a polarizing layer, a quantum dot-containing layer, and/or the like). In an embodiment, the film may be a light-blocking member, (for example, a light reflective layer, a light absorbing layer, and/or the like). In an embodiment, the film may be a protective member, (for example, an insulating layer, a dielectric layer, and/or the like).

### Electronic Apparatus

Some embodiments of the present disclosure relate to an electronic apparatus that includes the light-emitting device. For example, the electronic apparatus may be a light-emitting apparatus, an authentication apparatus, and/or the like.

The electronic apparatus (for example, a light-emitting apparatus) may include, in addition to the light-emitting device, i) a color filter, ii) a color conversion layer, or iii) a color filter and a color conversion layer. The color filter and/or the color conversion layer may be located along or in at least one direction in which light emitted from the light-emitting device travels. For example, the light emitted from the light-emitting device may be blue light or white light. For details on the light-emitting device, related description provided above may be referred to. In some embodiments, the color conversion layer may include a quantum dot. The quantum dot may be, for example, a quantum dot as described herein.

The electronic apparatus may include a first substrate. The first substrate may include a plurality of subpixel areas. In an embodiment, the color filter may include a plurality of color filter areas respectively corresponding to the subpixel areas. In an embodiment, the color conversion layer may include a plurality of color conversion areas respectively corresponding to the subpixel areas.

In an embodiment, a pixel defining layer may be located among the subpixel areas to define each of the subpixel areas.

In an embodiment, the color filter may include a plurality of color filter areas and light-shielding patterns located among the color filter areas. In an embodiment, the color conversion layer may include a plurality of color conversion areas and light-shielding patterns located among the color conversion areas.

The plurality of color filter areas (or the plurality of color conversion areas) may include a first area emitting a first color light, a second area emitting a second color light, and/or a third area emitting a third color light. In an embodiment, the first color light, the second color light, and/or the third color light may have different maximum emission wavelengths from one another. For example, the first color light may be red light, the second color light may be green light, and the third color light may be blue light. In an embodiment, the plurality of color filter areas (or the plurality of color conversion areas) may include quantum dots. In an embodiment, the first area may include a red quantum dot, the second area may include a green quantum dot, and the third area may not include (e.g., may exclude) a quantum dot. For details on the quantum dot, related descriptions provided herein may be referred to. In an embodiment, the first area, the second area, and/or the third area may each include a scatterer.

For example, the light-emitting device may be configured to emit a first light, the first area may be configured to absorb the first light and emit a first-first color light, the second area may be configured to absorb the first light and emit a second-first color light, and the third area may be configured to absorb the first light and emit a third-first color light. In this regard, the first-first color light, the second-first color light, and the third-first color light may have different maximum emission wavelengths. In an embodiment, the first light may be blue light, the first-first color light may be red light, the second-first color light may be green light, and the third-first color light may be blue light.

The electronic apparatus may include a thin-film transistor, in addition to the light-emitting device as described above. The thin-film transistor may include a source electrode, a drain electrode, and an activation layer, wherein one of the source electrode or the drain electrode may be electrically connected to one of the first electrode or the second electrode of the light-emitting device.

The thin-film transistor may further include a gate electrode, a gate insulating film, and/or the like.

The activation layer may include crystalline silicon, amorphous silicon, an organic semiconductor, an oxide semiconductor, and/or the like.

The electronic apparatus may include a sealing portion for sealing the light-emitting device. The sealing portion may be located between the color filter and/or the color conversion layer and the light-emitting device. The sealing portion allows light to be directed away from the light-emitting device, and concurrently (e.g., simultaneously) prevents or reduces ambient air and moisture from penetrating into the light-emitting device. The sealing portion may be a sealing substrate including a transparent glass substrate or a plastic substrate. The sealing portion may be a thin-film encapsulation layer including at least one layer of an organic layer and/or an inorganic layer. When the sealing portion is a thin film encapsulation layer, the electronic apparatus may be flexible.

One or more suitable functional layer(s) may be located on the sealing portion, in addition to the color filter and/or the color conversion layer, according to the required and/or desired function of the electronic apparatus. Examples of the functional layers may include a touch screen layer, a polarizing layer, and/or the like. The touch screen layer may be a pressure-sensitive touch screen layer, a capacitive touch screen layer, or an infrared touch screen layer. The authentication apparatus may be, for example, a biometric authentication apparatus that authenticates an individual by utilizing biometric information of a living body (for example, fingertips, pupils, and/or the like).

The authentication apparatus may include, in addition to the light-emitting device as described above, a biometric information collector.

The electronic apparatus may be utilized with or applied to one or more suitable displays, light sources, lighting, personal computers (for example, a mobile personal computer), mobile phones, digital cameras, electronic organizers, electronic dictionaries, electronic game machines, medical instruments (for example, electronic thermometers, sphygmomanometers, blood glucose meters, pulse measurement devices, pulse wave measurement devices, electrocardiogram displays, ultrasonic diagnostic devices, or endoscope displays), fish finders, one or more suitable measuring instruments, meters (for example, meters for a vehicle, an aircraft, and a vessel), projectors, and/or the like.

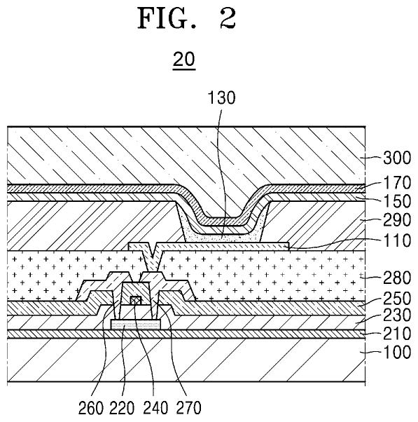

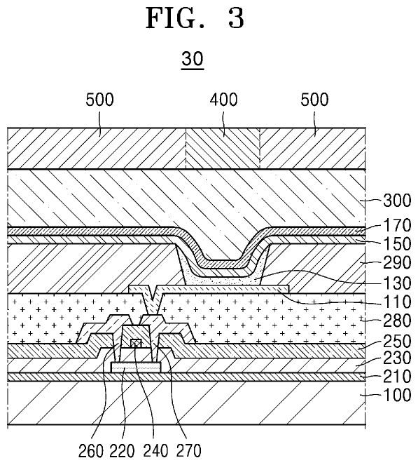

### Description of FIGS. 2 and 3

FIG. 2 is a schematic cross-sectional view showing a light-emitting apparatus 20 according to an embodiment of the present disclosure.

The light-emitting apparatus 20 includes a substrate 100, a thin-film transistor (TFT), a light-emitting device, and an encapsulation portion 300 that seals the light-emitting device.

The substrate 100 may be a flexible substrate, a glass substrate, or a metal substrate. A buffer layer 210 may be located at (e.g., on), the substrate 100. The buffer layer 210 may prevent or reduce penetration of impurities through the substrate 100 and may provide a flat surface on the substrate 100.

The TFT may be located at (e.g., on), the buffer layer 210. The TFT may include an activation layer 220, a gate electrode 240, a source electrode 260, and a drain electrode 270.

The activation layer 220 may include an inorganic semiconductor such as silicon or polysilicon, an organic semiconductor, or an oxide semiconductor. The activation layer 220 may include a source region, a drain region, and a channel region.

A gate insulating film 230 may be located at (e.g., on), the activation layer 220. The gate insulating film 230 may be configured to insulate the activation layer 220 from the gate electrode 240. The gate electrode 240 may be located at (e.g., on), the gate insulating film 230.

An interlayer insulating film 250 may be located at (e.g., on), the gate electrode 240. The interlayer insulating film 250 may be configured to insulate components of the light-emitting apparatus 20 from one another. The interlayer insulating film 250 may be located between the gate electrode 240 and the source electrode 260 and/or between the gate electrode 240 and the drain electrode 270, e.g., to insulate them from one another.

The source electrode 260 and the drain electrode 270 may be located at (e.g., on), the interlayer insulating film 250. The interlayer insulating film 250 and the gate insulating film 230 may be formed to expose the source region and/or the drain region of the activation layer 220. In an embodiment, the source electrode 260 and/or the drain electrode 270 may contact exposed portions of the source region and/or the drain region of the activation layer 220.

The TFT is electrically connected to a light-emitting device to provide electrical power to, or drive, the light-emitting device, and is covered and protected by a passivation layer 280. The passivation layer 280 may include an inorganic insulating film, an organic insulating film, or a combination thereof. The light-emitting device is located at (e.g., on), the passivation layer 280. The light-emitting device may include the first electrode 110, the interlayer 130, and the second electrode 150.

The first electrode 110 may be located at (e.g., on), the passivation layer 280. In an embodiment, the passivation layer 280 may be configured or located to expose a portion of the drain electrode 270. In an embodiment, the passivation layer 280 may cover a portion, (i.e., not the entire structure), of the drain electrode 270. The first electrode 110 may be configured or connected to the exposed portion of the drain electrode 270.

A pixel defining layer 290 that includes an insulating material may be located at (e.g., on), the first electrode 110. The pixel defining layer 290 may expose a region of the first electrode 110, and an interlayer 130 may be formed in an exposed region of the first electrode 110. The pixel defining layer 290 may be a polyimide or polyacrylic organic film. In an embodiment, at least some layers of the interlayer 130 may extend beyond the upper portion of the pixel defining layer 290 to be configured or located as (or in the form of), a common layer.

The second electrode 150 may be located at (e.g., on), the interlayer 130, and a capping layer 170 may be located at (e.g., on), the second electrode 150. The capping layer 170 may be configured, or formed, to cover the second electrode 150.

The encapsulation portion 300 may be located at (e.g., on), the capping layer 170. The encapsulation portion 300 may be located at (e.g., on), the light-emitting device to protect the light-emitting device from moisture and/or oxygen. The encapsulation portion 300 may include: an inorganic film including silicon nitride (SiNx), silicon oxide (SiOx), indium tin oxide (ITO), indium zinc oxide (IZO), or a combination thereof; an organic film including polyethylene terephthalate, polyethylene naphthalate, polycarbonate, polyimide, polyethylene sulfonate, polyoxymethylene, polyarylate, hexamethyldisiloxane, an acrylic resin (for example, polymethyl methacrylate, polyacrylic acid, and/or the like), an epoxy-based resin (for example, aliphatic glycidyl ether (AGE), and/or the like), or a combination thereof; or any combination of the inorganic films and the organic films.

FIG. 3 is a schematic cross-sectional view showing a light-emitting apparatus 30 according to an embodiment of the present disclosure.

The light-emitting apparatus 30 include all components as described for the light-emitting apparatus 20 of FIG. 2, and includes a light-shielding pattern 500 and a functional region 400 that are each located at (e.g., on), the encapsulation portion 300 of the light-emitting apparatus 30. The functional region 400 may be i) a color filter area, ii) a color conversion area, or iii) a combination of the color filter area and the color conversion area. In an embodiment, the light-emitting device included in the light-emitting apparatus 30 may be a tandem light-emitting device.

### Operating Characteristics of the Light-Emitting Device

The light-emitting device disclosed herein may be utilized at a driving voltage of about 4 V to about 6.5 V; or about 4.7 V to about 5.7 V; or about 4.9 V to about 5.3 V.

The light-emitting device disclosed herein may be characterized by a luminance of about 2600 to about 3500; or about 3000 to about 3300; or about 3100 to about 3280, where luminance is reported as candela per square meter (cd/m²).

The light-emitting device disclosed herein may be characterized by an efficiency of about 5.4 to about 9; or about 6 to about 7.5; or about 6.1 to about 6.6, where efficiency is reported as candela per ampere (cd/A).

The light-emitting device disclosed herein may be characterized by a half lifespan of about 280 hours (h) to about 900 h; or about 300 h to about 780 h; or about 300 h to about 660 h, where half lifespan is reported at 100 milliampere per square centimeter (mA/cm²).

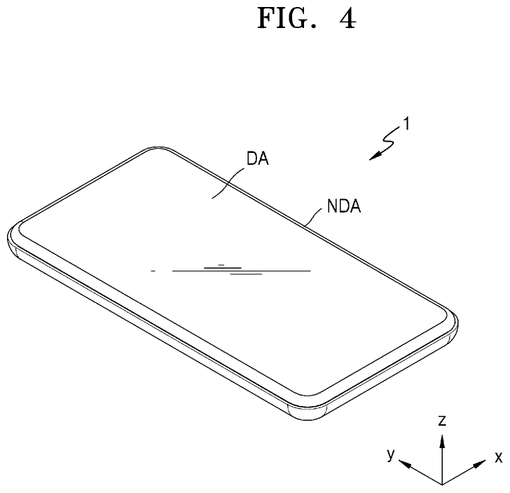

### Description of FIG. 4

FIG. 4 is a schematic perspective view of an electronic equipment 1 that includes a light-emitting device according to an embodiment of the present disclosure. The electronic equipment 1 may be a device apparatus that displays a moving image or still image, and/or a portable electronic equipment, non-limiting examples of which include a mobile phone, a smart phone, a tablet personal computer (PC), a mobile communication terminal, an electronic notebook, an electronic book, a portable multimedia player (PMP), a navigation, and/or a ultra mobile PC (UMPC). In an embodiment, the electronic equipment 1 may be one or more suitable products, such as a television, a laptop, a monitor, a billboard and/or a component of the Internet of things (IOT). In an embodiment, the electronic equipment 1 may be a component in a device apparatus as described herein. In some embodiments, the electronic equipment 1 may be a wearable device, such as a smart watch, a watch phone, a glasses-type or kind display, or a head mounted display (HMD), and/or a part of the wearable device. However, embodiments are not limited thereto. For example, the electronic apparatus 1 may be a center information display (CID) on an instrument panel and a center fascia or dashboard of a vehicle, a room mirror display instead of a side mirror of a vehicle, an entertainment display for the rear seat of a car or a display placed on the back of the front seat, head up display (HUD) installed in front of a vehicle or projected on a front window glass, or a computer generated hologram augmented reality head up display (CGH AR HUD).

FIG. 4 illustrates an embodiment in which the electronic equipment 1 is a smartphone. The electronic equipment 1 may include a display area DA and a non-display area NDA that is external to, or outside, the display area DA. A display device may implement an image through an array of a plurality of pixels that are two-dimensionally arranged at (e.g., on or in), the display area DA.

The non-display area NDA does not display an image, and may entirely surround the display area DA. A driver for providing electrical signals and/or power to display devices at (e.g., on), the display area DA may be arranged at (e.g., on), the non-display area NDA. In an embodiment, a pad, which is an area to which an electronic element or a printing circuit board may be electrically connected, may be arranged at (e.g., on), the non-display area NDA.

In the electronic equipment 1, a length in the x-axis direction and a length in the y-axis direction may be different from each other. For example, as shown in FIG. 4, the length in the x-axis direction may be shorter than the length in the y-axis direction. In some embodiments, the length in the x-axis direction may be the same as the length in the y-axis direction. In some embodiments, the length in the x-axis direction may be longer than the length in the y-axis direction.

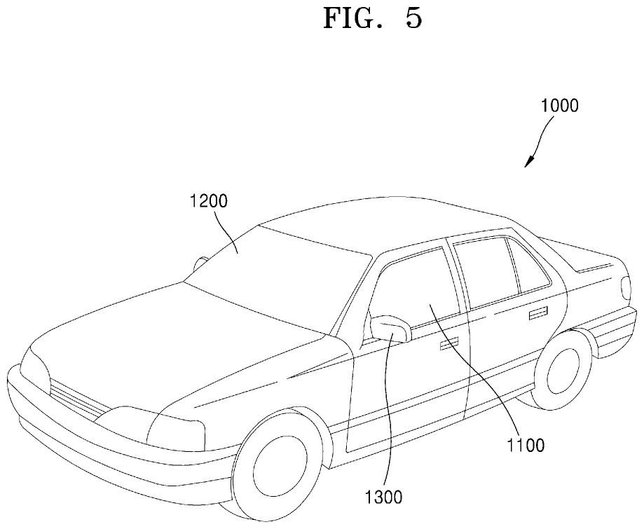

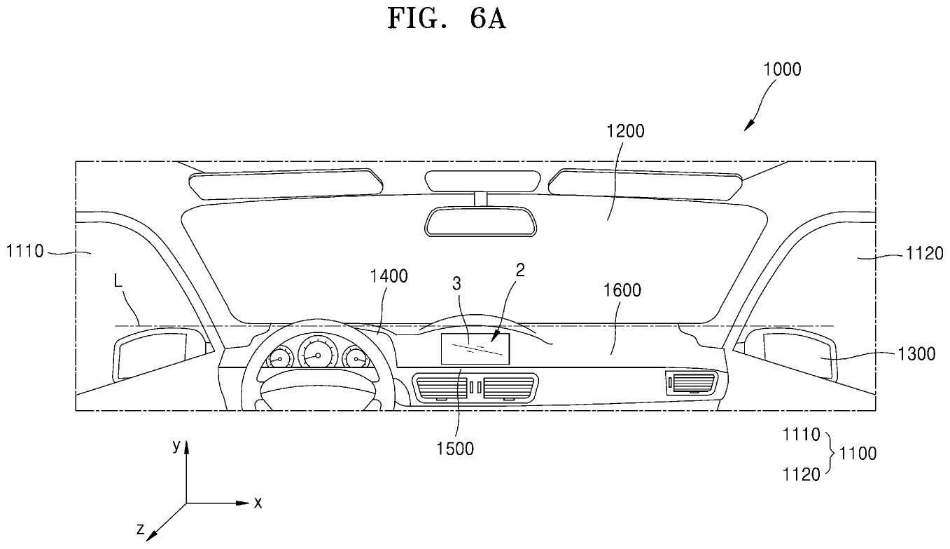

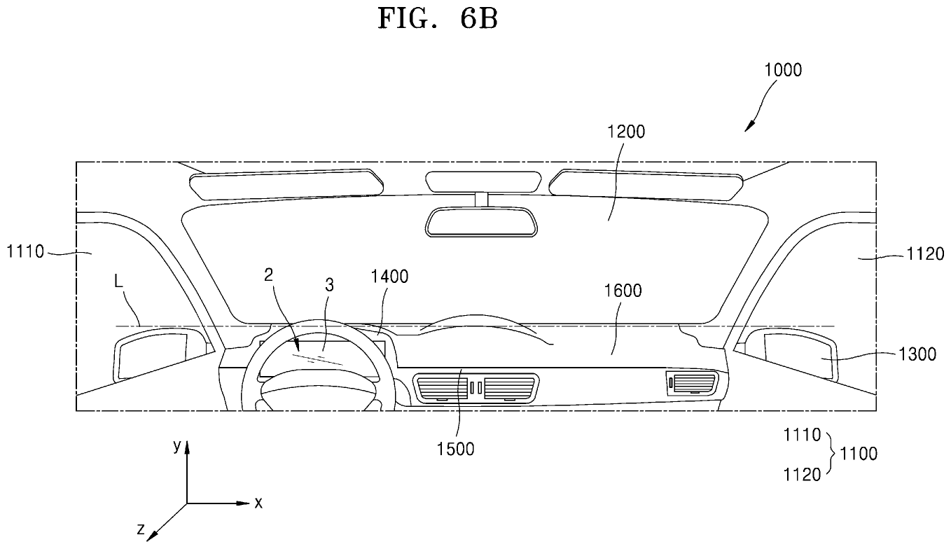

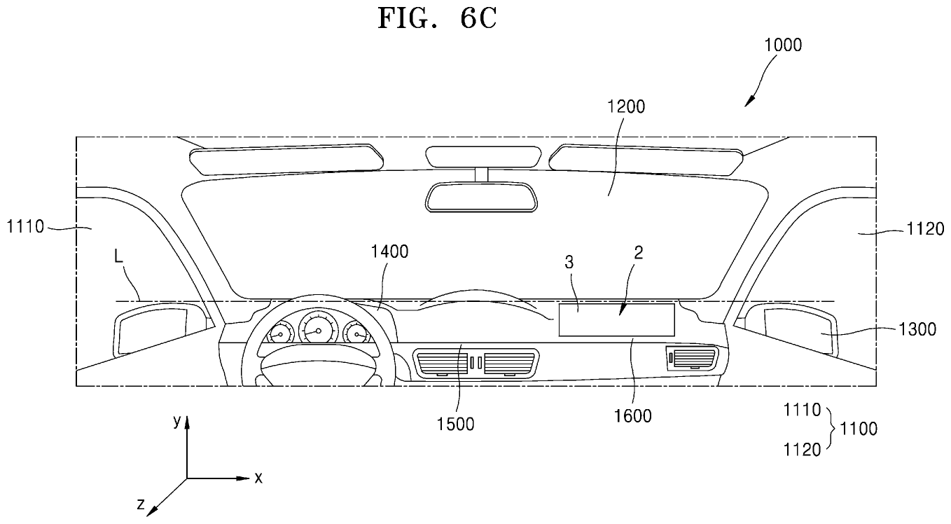

### Description of FIGS. 5 and 6A to 6C

FIG. 5 is a schematic perspective view of the exterior of a vehicle 1000.

FIGS. 6A to 6C are each a diagram schematic view of the interior of the vehicle 1000 that has an electronic equipment including the light-emitting device according to one or more suitable embodiments.

Referring to FIGS. 5, 6A, 6B, and 6C, the vehicle 1000 may refer to one or more suitable apparatus(es) for moving a subject from a departure point to a destination. The subject may be a human, an object, or an animal. The vehicle 1000 may include an automobile traveling on a road or track, a vessel moving over a sea or river, an airplane flying in the sky utilizing the action of air, and/or the like.

The vehicle 1000 may travel on a road or a track. The vehicle 1000 may move in a set or predetermined direction according to the rotation of at least one wheel. For example, the vehicle 1000 may include a three-wheeled or four-wheeled vehicle, a construction machine, a two-wheeled vehicle, a prime mover device, a bicycle, and a train running on a track.

The vehicle 1000 may include a body having an interior and an exterior, and a chassis in which mechanical apparatuses necessary for driving are installed as other parts except for the body. The exterior of the vehicle body may include a front panel, a bonnet or hood, a roof panel, a rear panel, a trunk, a filler provided at a boundary between doors, and/or the like. The chassis of the vehicle 1000 may include a power generating device, a power transmitting device, a driving device, a steering device, a braking device, a suspension device, a transmission device, a fuel device, front and rear wheels, left and right wheels, and/or the like.

The vehicle 1000 may include a side window glass 1100, a front window glass 1200, a side mirror 1300, a cluster 1400, a center fascia 1500, a passenger seat dashboard 1600, and a display device 2.

The side window glass 1100 and the front window glass 1200 may be partitioned by a filler arranged between the side window glass 1100 and the front window glass 1200. The side window glass 1100 may be installed on the side of the vehicle 1000. In an embodiment, the side window glass 1100 may be installed on a door of the vehicle 1000. A plurality of side window glasses 1100 may be provided and may face each other. In an embodiment, the side window glass 1100 may include a first side window glass 1110 and a second side window glass 1120. In an embodiment, the first side window glass 1110 may be arranged adjacent to the cluster 1400. The second side window glass 1120 may be arranged adjacent to the passenger seat dashboard 1600.

In an embodiment, the side window glasses 1100 may be spaced apart from each other in the x-direction or the −x-direction. For example, the first side window glass 1110 and the second side window glass 1120 may be spaced apart from each other in the x direction or the −x direction. In other words, an imaginary straight line L connecting the side window glasses 1100 may extend in the x-direction or the −x-direction. For example, an imaginary straight line L connecting the first side window glass 1110 and the second side window glass 1120 to each other may extend in the x direction or the −x direction.

The front window glass 1200 may be installed in the front of the vehicle 1000. The front window glass 1200 may be arranged between the side window glasses 1100 facing each other.

The side mirror 1300 may provide a rear view of the vehicle 1000. The side mirror 1300 may be installed on the exterior of the vehicle body. In one embodiment, a plurality of side mirrors 1300 may be provided. Any one of the plurality of side mirrors 1300 may be arranged outside the first side window glass 1110. The other one of the plurality of side mirrors 1300 may be arranged outside the second side window glass 1120.

The cluster 1400 may be arranged in front of the steering wheel. The cluster 1400 may include a tachometer, a speedometer, a coolant thermometer, a fuel gauge turn indicator, a high beam indicator, a warning lamp, a seat belt warning lamp, an odometer, a hodometer, an automatic shift selector indicator lamp, a door open warning lamp, an engine oil warning lamp, and/or a low fuel warning light.

The center fascia 1500 may include a control panel on which a plurality of buttons for adjusting an audio device, an air conditioning device, and a heater of a seat are disposed. The center fascia 1500 may be arranged on one side of the cluster 1400.

A passenger seat dashboard 1600 may be spaced apart from the cluster 1400 with the center fascia 1500 arranged therebetween. In an embodiment, the cluster 1400 may be arranged to correspond to a driver seat, and the passenger seat dashboard 1600 may be disposed to correspond to a passenger seat. In an embodiment, the cluster 1400 may be adjacent to the first side window glass 1110, and the passenger seat dashboard 1600 may be adjacent to the second side window glass 1120.

In an embodiment, the vehicle 1000 may include the display device 2 that includes a display panel 3 configured to display an image. The display device 2 may be arranged inside the vehicle 1000. In an embodiment, the display device 2 may be arranged between the side window glasses 1100 facing each other. The display device 2 may be arranged at (e.g, on), at least one of the cluster 1400, the center fascia 1500, and the passenger seat dashboard 1600.

The display device 2 may include an organic light-emitting display device, an inorganic electroluminescent (EL) display device, a quantum dot display device, and/or the like. Hereinafter, as the display device 2 according to an embodiment, an organic light-emitting display device display including the light-emitting device according to the disclosure will be described as an example, but one or more suitable types (kinds) of display devices as described above may be utilized in embodiments of the present disclosure.

Referring to FIG. 6A, the display device 2 may be arranged on the center fascia 1500. In an embodiment, the display device 2 may display navigation information. In an embodiment, the display device 2 may display audio, video, or information regarding vehicle settings.

Referring to FIG. 6B, the display device 2 may be arranged on the cluster 1400. When the display device 2 is arranged on the cluster 1400, the cluster 1400 may display driving information and/or the like through the display device 2. For example, the cluster 1400 may be implemented digitally. The digital cluster 1400 may display vehicle information and driving information as images. For example, a needle and a gauge of a tachometer and one or more suitable warning light icons may be displayed by a digital signal.

Referring to FIG. 6C, the display device 2 may be arranged on the dashboard 1600 of the passenger seat. The display device 2 may be embedded in the passenger seat dashboard 1600 or arranged on the passenger seat dashboard 1600.

In an embodiment, the display device 2 arranged on the dashboard 1600 for the passenger seat may display an image related to information displayed on the cluster 1400 and/or information displayed on the center fascia 1500. In some embodiments, the display device 2 arranged on the passenger seat dashboard 1600 may display information different from information displayed on the cluster 1400 and/or information displayed on the center fascia 1500.

### Manufacturing Method

The respective layers included in the hole transport region, the emission layer, and/or the electron transport region may be formed by utilizing one or more suitable methods selected from vacuum deposition, spin coating, casting, Langmuir-Blodgett (LB) deposition, ink-jet printing, laser-printing, and/or laser-induced thermal imaging.

The layers of the hole transport region, the emission layer, and/or the electron transport region may be formed by vacuum deposition at a deposition temperature of about 100° C. to about 500° C., a vacuum degree of about 10⁻⁸ torr to about 10⁻³ torr, and a deposition speed of about 0.01 Å/sec to about 100 Å/sec, depending on a material to be included in a layer to be formed and the structure of a layer to be formed.

### Definition of Terms

The term “C₃-C₆₀ carbocyclic group” as utilized herein refers to a cyclic group consisting of carbon only as a ring-forming atom and having three to sixty carbon atoms, and the term “C₁-C₆₀ heterocyclic group” as utilized herein refers to a cyclic group that has one to sixty carbon atoms and further has, in addition to carbon, a heteroatom as a ring-forming atom. The C₃-C₆₀ carbocyclic group and the C₁-C₆₀ heterocyclic group may each be a monocyclic group consisting of one ring or a polycyclic group in which two or more rings are condensed with each other. For example, the C₁-C₆₀ heterocyclic group has 3 to 61 ring-forming atoms.

The term “cyclic group” as utilized herein may include the C₃-C₆₀ carbocyclic group, and the C₁-C₆₀ heterocyclic group.

The term “π electron-rich C₃-C₆₀ cyclic group” as utilized herein refers to a cyclic group that has three to sixty carbon atoms and does not include *—N═* as a ring-forming moiety, and the term “π electron-deficient nitrogen-containing C₁-C₆₀ cyclic group” as utilized herein refers to a heterocyclic group that has one to sixty carbon atoms and includes *—N═*′ as a ring-forming moiety.

For example,
the C₃-C₆₀ carbocyclic group may be i) a group T1 or ii) a condensed cyclic group in which two or more groups T1 are condensed with each other (for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),
the C₁-C₆₀ heterocyclic group may be i) a group T2, ii) a condensed cyclic group in which two or more groups T2 are condensed with each other, or iii) a condensed cyclic group in which at least one group T2 and at least one group T1 are condensed with each other (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),
the π electron-rich C₃-C₆₀ cyclic group may be i) a group T1, ii) a condensed cyclic group in which two or more groups T1 are condensed with each other, iii) a group T3, iv) a condensed cyclic group in which two or more groups T3 are condensed with each other, or v) a condensed cyclic group in which at least one group T3 and at least one group T1 are condensed with each other (for example, the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, etc.),
the π electron-deficient nitrogen-containing C₁-C₆₀ cyclic group may be i) a group T4, ii) a condensed cyclic group in which two or more groups T4 are condensed with each other, iii) a condensed cyclic group in which at least one group T4 and at least one group T1 are condensed with each other, iv) a condensed cyclic group in which at least one group T4 and at least one group T3 are condensed with each other, or v) a condensed cyclic group in which at least one group T4, at least one group T1, and at least one group T3 are condensed with one another (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),
the group T1 may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or a bicyclo[2.2.1]heptane) group, a norbornene group, a bicyclo[1.1.1]pentane group, a bicyclo[2.1.1]hexane group, a bicyclo[2.2.2]octane group, or a benzene group,
the group T2 may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group,
the group T3 may be a furan group, a thiophene group, a 1H-pyrrole group, a silole group, or a borole group, and
the group T4 may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The terms “the cyclic group, the C₃-C₆₀ carbocyclic group, the C₁-C₆₀ heterocyclic group, the π electron-rich C₃-C₆₀ cyclic group, or the π electron-deficient nitrogen-containing C₁-C₆₀ cyclic group” as utilized herein refer to a group condensed to any cyclic group, a monovalent group, or a polyvalent group (for example, a divalent group, a trivalent group, a tetravalent group, etc.) according to the structure of a formula for which the corresponding term is utilized. For example, the “benzene group” may be a benzo group, a phenyl group, a phenylene group, and/or the like, which may be easily understood by one of ordinary skill in the art according to the structure of a formula including the “benzene group.”

Depending on context, a divalent group may refer or be a polyvalent group (e.g., trivalent, tetravalent, etc., and not just divalent) per, e.g., the structure of a formula in connection with which of the terms are utilized.

Examples of the monovalent C₃-C₆₀ carbocyclic group and the monovalent C₁-C₆₀ heterocyclic group are a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group. Examples of the divalent C₃-C₆₀ carbocyclic group and the monovalent C₁-C₆₀ heterocyclic group are a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a substituted or unsubstituted divalent non-aromatic condensed heteropolycyclic group.

The term “C₁-C₆₀ alkyl group” as utilized herein refers to a linear or branched aliphatic hydrocarbon monovalent group that has one to sixty carbon atoms, and specific examples thereof are a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, and a tert-decyl group. The term “C₁-C₆₀ alkylene group” as utilized herein refers to a divalent group having the same structure as the C₁-C₆₀ alkyl group.

The term “C₂-C₆₀ alkenyl group” as utilized herein refers to a monovalent hydrocarbon group having at least one carbon-carbon double bond in the middle or at the terminus of the C₂-C₆₀ alkyl group, and examples thereof are an ethenyl group, a propenyl group, and a butenyl group. The term “C₂-C₆₀ alkenylene group” as utilized herein refers to a divalent group having the same structure as the C₂-C₆₀ alkenyl group.

The term “C₂-C₆₀ alkynyl group” as utilized herein refers to a monovalent hydrocarbon group having at least one carbon-carbon triple bond in the middle or at the terminus of the C₂-C₆₀ alkyl group, and examples thereof are an ethynyl group, a propynyl group, and/or the like. The term “C₂-C₆₀ alkynylene group” as utilized herein refers to a divalent group having the same structure as the C₂-C₆₀ alkynyl group.

The term “C₁-C₆₀ alkoxy group” as utilized herein refers to a monovalent group represented by —OA₁₀₁ (wherein A₁₀₁ is the C₁-C₆₀ alkyl group), and examples thereof include a methoxy group, an ethoxy group, and an isopropyloxy group.

The term “C₃-C₁₀ cycloalkyl group” as utilized herein refers to a monovalent saturated hydrocarbon cyclic group having 3 to 10 carbon atoms, and examples thereof are a cyclopropyl group, a cyclobutyl group, a cyclopentyl group, a cyclohexyl group, a cycloheptyl group, a cyclooctyl group, an adamantanyl group, a norbornanyl group (or bicyclo[2.2.1]heptyl group), a bicyclo[1.1.1]pentyl group, a bicyclo[2.1.1]hexyl group, and a bicyclo[2.2.2]octyl group. The term “C₃-C₁₀ cycloalkylene group” as utilized herein refers to a divalent group having the same structure as the C₃-C₁₀ cycloalkyl group.

The term “C₁-C₁₀ heterocycloalkyl group” as utilized herein refers to a monovalent cyclic group of 1 to 10 carbon atoms, further including, in addition to carbon atoms, at least one heteroatom, as ring-forming atoms, and specific examples are a 1,2,3,4-oxatriazolidinyl group, a tetrahydrofuranyl group, and a tetrahydrothiophenyl group. The term “C₁-C₁₀ heterocycloalkylene group” as utilized herein refers to a divalent group having the same structure as the C₁-C₁₀ heterocycloalkyl group.

The term C₃-C₁₀ cycloalkenyl group utilized herein refers to a monovalent cyclic group that has three to ten carbon atoms and at least one carbon-carbon double bond in the ring thereof and no aromaticity, and specific examples thereof are a cyclopentenyl group, a cyclohexenyl group, and a cycloheptenyl group. The term “C₃-C₁₀ cycloalkenylene group” as utilized herein refers to a divalent group having the same structure as the C₃-C₁₀ cycloalkenyl group.

The term “C₁-C₁₀ heterocycloalkenyl group” as utilized herein refers to a monovalent cyclic group of 1 to 10 carbon atoms, further including, in addition to carbon atoms, at least one heteroatom, as ring-forming atoms, and having at least one carbon-carbon double bond in the cyclic structure thereof. Examples of the C₁-C₁₀ heterocycloalkenyl group include a 4,5-dihydro-1,2,3,4-oxatriazolyl group, a 2,3-dihydrofuranyl group, and a 2,3-dihydrothiophenyl group. The term “C₁-C₁₀ heterocycloalkenylene group” as utilized herein refers to a divalent group having the same structure as the C₁-C₁₀ heterocycloalkenyl group.

The term “C₆-C₆₀ aryl group” as utilized herein refers to a monovalent group having a carbocyclic aromatic system of 6 to 60 carbon atoms, and the term “C₆-C₆₀ arylene group” as utilized herein refers to a divalent group having a carbocyclic aromatic system of 6 to 60 carbon atoms. Examples of the C₆-C₆₀ aryl group are a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, and an ovalenyl group. When the C₆-C₆₀ aryl group and the C₆-C₆₀ arylene group each include two or more rings, the rings may be condensed with each other.

The term “C₁-C₆₀ heteroaryl group” as utilized herein refers to a monovalent group having a heterocyclic aromatic system of 1 to 60 carbon atoms, further including, in addition to carbon atoms, at least one heteroatom, as ring-forming atoms. The term “C₁-C₆₀ heteroarylene group” as utilized herein refers to a divalent group having a heterocyclic aromatic system of 1 to 60 carbon atoms, further including, in addition to carbon atoms, at least one heteroatom, as ring-forming atoms. Examples of the C₁-C₆₀ heteroaryl group are a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, and a naphthyridinyl group.

When the C₁-C₆₀ heteroaryl group and the C₁-C₆₀ heteroarylene group each include two or more rings, the rings may be condensed with each other.

The term “monovalent non-aromatic condensed polycyclic group” as utilized herein refers to a monovalent group (for example, having 8 to 60 carbon atoms) having two or more rings condensed to each other, only carbon atoms as ring-forming atoms, and no aromaticity in its entire molecular structure. Examples of the monovalent non-aromatic condensed polycyclic group are an indenyl group, a fluorenyl group, a spiro-bifluorenyl group, a benzofluorenyl group, an indenophenanthrenyl group, and an indeno anthracenyl group. The term “divalent non-aromatic condensed polycyclic group” as utilized herein refers to a divalent group having the same structure as the monovalent non-aromatic condensed polycyclic group described above.

The term “monovalent non-aromatic condensed heteropolycyclic group” as utilized herein refers to a monovalent group (for example, having 1 to 60 carbon atoms) having two or more rings condensed to each other, further including, in addition to carbon atoms, at least one heteroatom, as ring-forming atoms, and having non-aromaticity in its entire molecular structure. Examples of the monovalent non-aromatic condensed heteropolycyclic group are a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indeno carbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphtho silolyl group, a benzofurodibenzofuranyl group, a benzofurodibenzothiophenyl group, and a benzothienodibenzothiophenyl group. The term “divalent non-aromatic condensed heteropolycyclic group” as utilized herein refers to a divalent group having the same structure as the monovalent non-aromatic condensed heteropolycyclic group described above.

The term “C₆-C₆₀ aryloxy group” as utilized herein indicates —OA₁₀₂ (wherein A₁₀₂ is a C₆-C₆₀ aryl group), and the term “C₆-C₆₀ arylthio group” as utilized herein indicates —SA₁₀₃ (wherein A₁₀₃ is a C₆-C₆₀ aryl group).

The term “C₇-C₆₀ arylalkyl group” utilized herein refers to -A₁₀₄A₁₀₅ (where A₁₀₄ may be a C₁-C₅₄ alkylene group, and A₁₀₅ may be a C₆-C₅₉ aryl group), and the term C₂-C₆₀ heteroarylalkyl group” utilized herein refers to -A₁₀₆A₁₀₇ (where A₁₀₆ may be a C₁-C₅₉ alkylene group, and A₁₀₇ may be a C₁-C₅₉ heteroaryl group).

The term “R₁₀ₐ” as utilized herein refers to:
deuterium, —F, —Cl, —Br, —I, a hydroxyl group, a cyano group, or a nitro group,
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or a combination thereof,
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or a combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
Q₁ to Q₃, Q₁₁ to Q₁₃, Q₂₁ to Q₂₃ and Q₃₁ to Q₃₃ utilized herein may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or a combination thereof; a C₇-C₆₀ arylalkyl group; or a C₂-C₆₀ heteroarylalkyl group.

The term “heteroatom” as utilized herein refers to any atom other than a carbon atom. Examples of the heteroatom are O, S, N, P, Si, B, Ge, Se, and any combinations thereof.

The term “third-row transition metal” utilized herein includes hafnium (Hf), tantalum (Ta), tungsten (W), rhenium (Re), osmium (Os), iridium (Ir), platinum (Pt), gold (Au), and/or the like.

“Ph” as utilized herein refers to a phenyl group, “Me” as utilized herein refers to a methyl group, “Et” as utilized herein refers to an ethyl group, “tert-Bu” or “But” as utilized herein refers to a tert-butyl group, and “OMe” as utilized herein refers to a methoxy group.

The term “biphenyl group” as utilized herein refers to “a phenyl group substituted with a phenyl group.” In other words, the “biphenyl group” is a substituted phenyl group having a C₆-C₆₀ aryl group as a substituent.

The term “terphenyl group” as utilized herein refers to “a phenyl group substituted with a biphenyl group.” In other words, the “terphenyl group” is a substituted phenyl group having, as a substituent, a C₆-C₆₀ aryl group substituted with a C₆-C₆₀ aryl group.

The term “substituted” as utilized herein, refers to that at least one hydrogen in a substituent or compound is deuterium, a halogen group, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 fluoroalkyl group, a cyano group, or a combination thereof.

In one example of the present disclosure, “substituted” refers to replacement of at least one hydrogen of a substituent or a compound by deuterium, a halogen, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C10 fluoroalkyl group, or a cyano group. In some embodiments, in specific examples of the present disclosure, “substituted” refers to replacement of at least on hydrogen of a substituent or a compound by deuterium, a halogen, a C1 to C20 alkyl group, a C6 to C30 aryl group, a C1 to C10 fluoroalkyl group, or a cyano group. In some embodiments, in specific examples of the present disclosure, “substituted” refers to replacement of at least one hydrogen of a substituent or a compound by deuterium, a halogen, a C1 to C5 alkyl group, a C6 to C18 aryl group, a C1 to C5 fluoroalkyl group, or a cyano group. In some embodiments, in specific examples of the present disclosure, “substituted” refers to replacement of at least one hydrogen of a substituent or a compound by deuterium, a cyano group, a halogen, a methyl group, an ethyl group, a propyl group, a butyl group, a phenyl group, a biphenyl group, a terphenyl group, a trifluoromethyl group, or a naphthyl group.

* and *′ as utilized herein, unless defined otherwise, each refer to a binding site to a neighboring atom (i.e., a point of attachment), in a corresponding formula or moiety.

In the present specification, the x-axis, y-axis, and z-axis are not limited to three axes in an orthogonal coordinate system, and may be interpreted in a broad sense including these axes. For example, the x-axis, y-axis, and z-axis may refer to those orthogonal to each other, or may refer to those in different directions that are not orthogonal to each other.

In the present disclosure, when particles are spherical, “diameter” indicates an average particle diameter, and when the particles are non-spherical, the “diameter” indicates a major axis length. The diameter (or size) of the particles may be measured utilizing a scanning electron microscope or a particle size analyzer. As the particle size analyzer, for example, HORIBA, LA-950 laser particle size analyzer, may be utilized.

When the size of the particles is measured utilizing a particle size analyzer, the average particle diameter (or size) is referred to as D50. D50 refers to the average diameter (or size) of particles whose cumulative volume corresponds to 50 vol % in the particle size distribution (e.g., cumulative distribution), and refers to the value of the particle size corresponding to 50% from the smallest particle when the total number of particles is 100% in the distribution curve accumulated in the order of the smallest particle size to the largest particle size.

Terms such as “substantially,” “about,” and “approximately” are used as relative terms and not as terms of degree, and are intended to account for the inherent deviations in measured or calculated values that would be recognized by those of ordinary skill in the art. They may be inclusive of the stated value and an acceptable range of deviation as determined by one of ordinary skill in the art, considering the limitations and error associated with measurement of that quantity. For example, “about” may refer to one or more standard deviations, or ±30%, 20%, 10%, 5% of the stated value.

Numerical ranges disclosed herein include and are intended to disclose all subsumed sub-ranges of the same numerical precision. For example, a range of “1.0 to 10.0” includes all subranges having a minimum value equal to or greater than 1.0 and a maximum value equal to or less than 10.0, such as, for example, 2.4 to 7.6.

Applicant therefore reserves the right to amend this specification, including the claims, to expressly recite any sub-range subsumed within the ranges expressly recited herein.

The light emitting device, electronic apparatus, electronic equipment and other relevant devices or components according to embodiments of the present disclosure described herein may be implemented utilizing any suitable hardware, firmware (e.g., an application-specific integrated circuit), software, or a combination of software, firmware, and hardware. For example, the various components of the light emitting device, electronic apparatus, and electronic equipment may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

Hereinafter, compounds and light-emitting devices according to embodiments will be described in more detail with reference to the following examples. These examples, however, are not in any sense to be interpreted as limiting the scope of the present disclosure. The wording “B was utilized instead of A” utilized in describing Synthesis Examples refers to that an identical molar equivalent of B was utilized in place of A.

### EXAMPLES

### Synthesis of Intermediates C1 to C4

### Synthesis of Intermediate C1

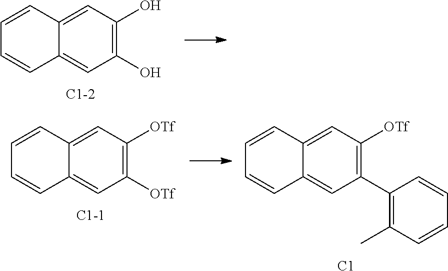

C1-2 (200 mmol, 1 eq), trifluoromethanesulfonic anhydride (100 mL, 3 eq), dichloromethane (800 mL), triethylamine (TEA) (110 mL, 5 eq) were put in a 1-neck-round flask and stirred for 24 hours at 0° C. After completion of the reaction, the reaction product was subjected to column separation with a mixture of dichloromethane and hexanes (MC/Hx)=1/10 after working up with ether/H₂O to obtain 133 mmol of C1-1 (yield: 66%).

Subsequently, C1-1 (133 mmol, 1 eq), o-tolyboronic acid (133 mmol, 1 eq), tetrakis(triphenylphosphine)palladium(0) (Pd(pph₃)₄) (6.7 mmol, 0.05 eq), K₂CO₃ (399 mmol, 3 eq), toluene (400 mL), ethanol (EtOH) (400 mL), and H₂O (200 mL) were put in a 1-neck-round flask and stirred for 1 hour at 110° C. After completion of the reaction, the reaction product was subjected to column separation with MC/Hx=1/10 after working up with ether/H₂O to obtain 57 mmol of Intermediate C1 (yield: 40%).

### Synthesis of Intermediate C2

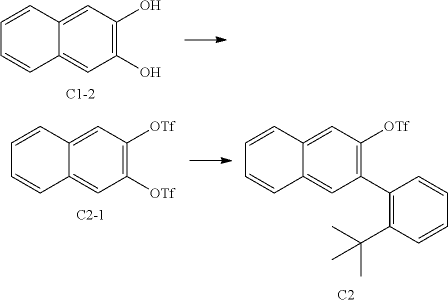

C2-2 (200 mmol, 1 eq), trifluoromethanesulfonic anhydride (100 mL, 3 eq), dichloromethane (800 mL), and triethylamine (110 mL, 5 eq) were put in a 1-neck-round flask and stirred for 24 hours at 0° C. After completion of the reaction, the reaction product was subjected to column separation with MC/Hx=1/10 after working up with ether/H₂O to obtain 133 mmol of C2-1 (yield: 66%).

Subsequently, C2-1 (133 mmol, 1 eq), (2-(tert-butyl)phenyl)boronic acid (133 mmol, 1 eq), Pd(pph₃)₄ (6.7 mmol, 0.05 eq), K₂CO₃ (399 mmol, 3 eq), toluene (400 mL), EtOH (400 mL), and H₂O (200 mL) were put in a 1-neck-round flask and stirred for 1 hour at 110° C. After completion of the reaction, the reaction product was subjected to column separation with hexanes (Hex)=100% after working up with ether/H₂O to obtain 57 mmol of Intermediate C2 (yield: 40%).

### Synthesis of Intermediate C3

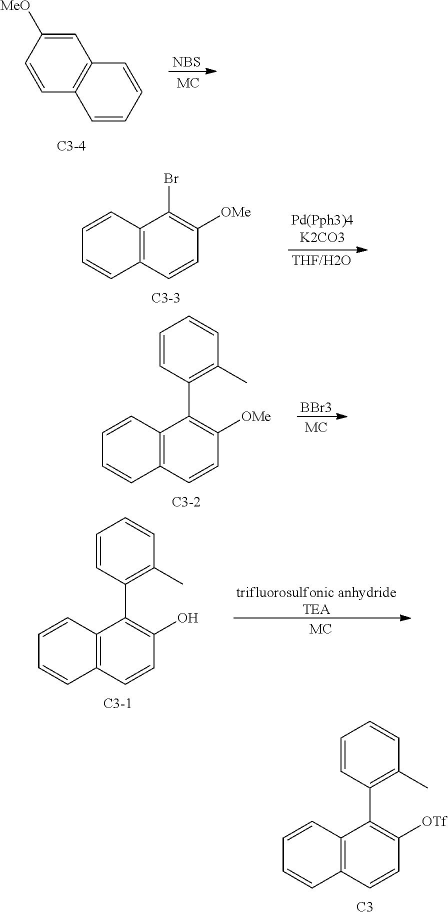

C3-4 (50 mmol, 1 eq), N-bromosuccinimide (NBS) (50 mmol, 1 eq), and MC (400 mL) were put in a 1-neck-round flask and stirred for 4 hours at 70° C. After completion of the reaction, the reaction product was subjected to column separation with Hx after working up with ether/H₂O to obtain 45 mmol of C3-3 (yield: 90%).

Subsequently, C3-3 (45 mmol, 1 eq), o-tolyboronic acid (45 mmol, 1 eq), Pd(pph₃)₄ (2.25 mmol, 0.05 eq), K₂CO₃ (135 mmol, 3 eq), and THF/H₂O (400/80 mL) were put in the reaction product and stirred for 24 hours at 80° C. After completion of the reaction, the reaction product was subjected to column separation with Hx after working up with ether/H₂O to obtain 36 mmol of C3-2 (yield: 80%).

C3-2 (36 mmol, 1 eq), BBr₃ (36 mmol, 1 eq), and MC (400 mL) were put in a 1-neck-round flask and stirred for 4 hours at 0° C. After completion of the reaction, the reaction product was subjected to column separation with MC/Hx=1/3 after working up with ether/H₂O to obtain 31.5 mmol of C3-1 (yield: 90%).

C3-1 (31.5 mmol, 1 eq), triflourosulfonic anhydride (63 mmol, 2 eq), TEA (63 mmol, 2 eq), and MC (400 mL) were put in a 1-neck-round flask and stirred for 2 hours at 0° C. After completion of the reaction, the reaction product was subjected to column separation with Hx after working up with ether/H₂O to obtain 26.8 mmol of Intermediate C3 (yield: 85%).

### Synthesis of Intermediate C4

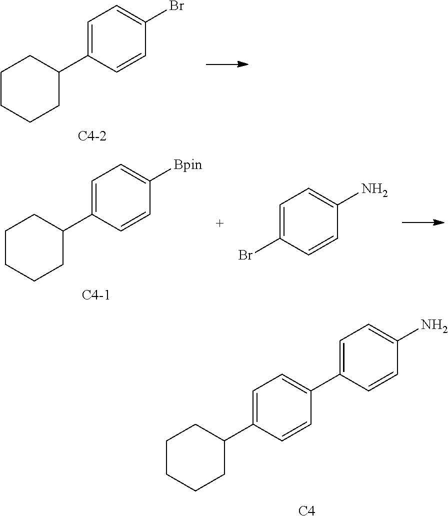

C4-2 (274 mmol, 1 eq), bis(pinacolato) diboron (330 mmol, 1.2 eq), 1,1′-bis(diphenylphosphino)ferrocene]dichloropalladium(II) (PdCl₂(dppf)) (13.7 mmol, 0.05 eq), potassium acetate (KOAc) (1000 mmol, 4 eq), and dioxane (1000 mL) were put in a 1-neck-round flask and stirred for 24 hours at 80° C. After completion of the reaction, the reaction product was subjected to column separation with MC/Hx=1/3 after working up with ether/H₂O to obtain 240 mmol of C4-1 (yield: 79%).

C4-1 (240 mmol, 1.2 eq), 4-bromoaniline (100 mmol, 1 eq), Pd(pph₃)₄ (5 mmol, 0.05 eq), K₂CO₃ (400 mmol, 4 eq), toluene (400 mL), EtOH (400 mL), and H₂O (200 mL) were put in a 1-neck-round flask and stirred for 1 hour at 110° C. After completion of the reaction, the reaction product was subjected to column separation with MC/Hx=1/3 after working up with ether/H₂O to obtain 96 mmol of Intermediate C4 (yield: 42%).

### Synthesis Example 1: Synthesis of Compound 1

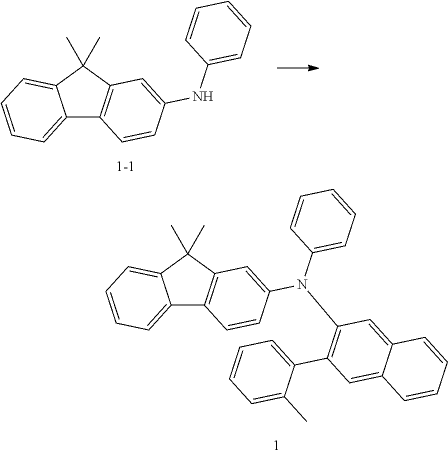 [SMILES: CC1(C)c2ccccc2-c2ccc(Nc3ccccc3)cc21 ; Cc1ccccc1-c1cc2ccccc2cc1N(c1ccccc1)c1ccc2c(c1)C(C)(C)c1ccccc1-2]

Compound 1-1 (10 mmol, 1 eq), Intermediate C1 (11 mmol, 1.1 eq), tris(dibenzylideneacetone)dipalladium(0) (Pd₂(dba)₃) (0.5 mmol, 0.05 eq), sodium tert-butoxide (t-BuONa) (20 mmol, 2 eq), tri-tert-butylphosphine (t-Bu₃P) (1 mmol, 0.1 eq), and toluene (150 mL) were put in a 1-neck-round flask and stirred for 1 hour at 110° C. After completion of the reaction, the reaction product was subjected to column separation with MC/Hx=1/7 after working up with ether/H₂O to obtain 7.6 mmol of Compound 1 (yield: 76%).

### Synthesis Example 2: Synthesis of Compound 2

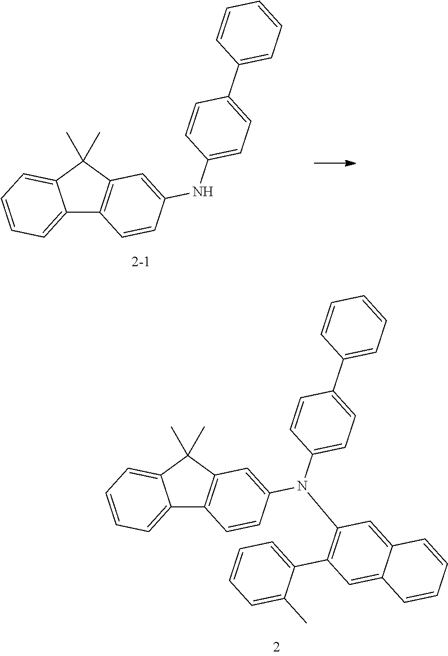 [SMILES: CC1(C)c2ccccc2-c2ccc(Nc3ccc(-c4ccccc4)cc3)cc21 ; Cc1ccccc1-c1cc2ccccc2cc1N(c1ccc(-c2ccccc2)cc1)c1ccc2c(c1)C(C)(C)c1ccccc1-2]

Compound 2-1 (10 mmol, 1 eq), Intermediate C1 (11 mmol, 1.1 eq), Pd₂(dba)₃ (0.5 mmol, 0.05 eq), t-BuONa (20 mmol, 2 eq), t-Bu₃P (1 mmol, 0.1 eq), and toluene (150 mL) were put in a 1-neck-round flask and stirred for 1 hour at 110° C. After completion of the reaction, the reaction product was subjected to column separation with MC/Hx=1/7 after working up with ether/H₂O to obtain 7.6 mmol of Compound 2 (yield: 76%).

### Synthesis Example 3: Synthesis of Compound 4

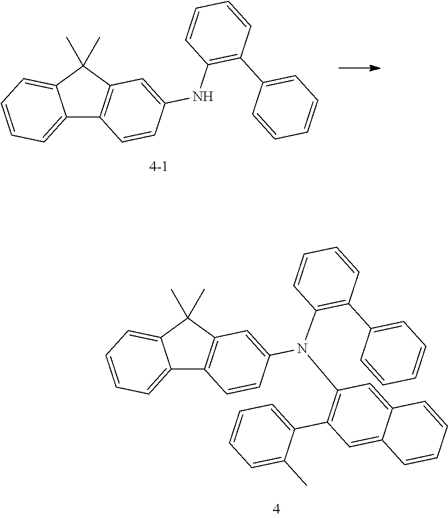 [SMILES: CC1(C)c2ccccc2-c2ccc(Nc3ccccc3-c3ccccc3)cc21 ; Cc1ccccc1-c1cc2ccccc2cc1N(c1ccc2c(c1)C(C)(C)c1ccccc1-2)c1ccccc1-c1ccccc1]

Compound 4-1 (10 mmol, 1 eq), Intermediate C1 (11 mmol, 1.1 eq), Pd₂(dba)₃ (0.5 mmol, 0.05 eq), t-BuONa (20 mmol, 2 eq), t-Bu₃P (1 mmol, 0.1 eq), and toluene (150 mL) were put in a 1-neck-round flask and stirred for 1 hour at 110° C. After completion of the reaction, the reaction product was subjected to column separation with MC/Hx=1/7 after working up with ether/H₂O to obtain 7.6 mmol of Compound 4 (yield: 76%).

### Synthesis Example 4: Synthesis of Compound 5

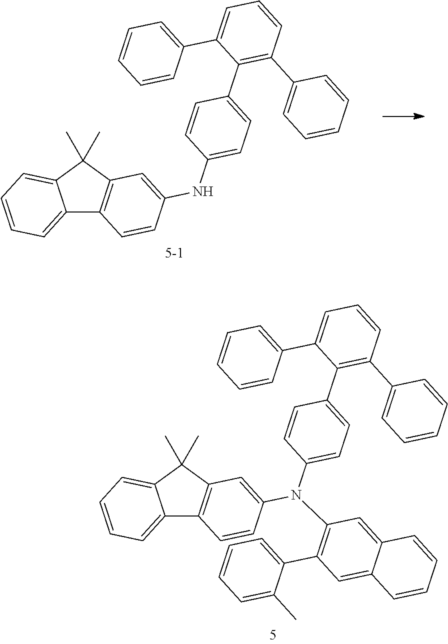 [SMILES: CC1(C)c2ccccc2-c2ccc(Nc3ccc(-c4c(-c5ccccc5)cccc4-c4ccccc4)cc3)cc21 ; Cc1ccccc1-c1cc2ccccc2cc1N(c1ccc(-c2c(-c3ccccc3)cccc2-c2ccccc2)cc1)c1ccc2c(c1)C(C)(C)c1ccccc1-2]

Compound 5-1 (10 mmol, 1 eq), Intermediate C1 (11 mmol, 1.1 eq), Pd₂(dba)₃ (0.5 mmol, 0.05 eq), t-BuONa (20 mmol, 2 eq), t-Bu₃P (1 mmol, 0.1 eq), and toluene (150 mL) were put in a 1-neck-round flask and stirred for 1 hour at 110° C. After completion of the reaction, the reaction product was subjected to column separation with MC/Hx=1/7 after working up with ether/H₂O to obtain 7.6 mmol of Compound 5 (yield: 76%).

### Synthesis Example 5: Synthesis of Compound 6

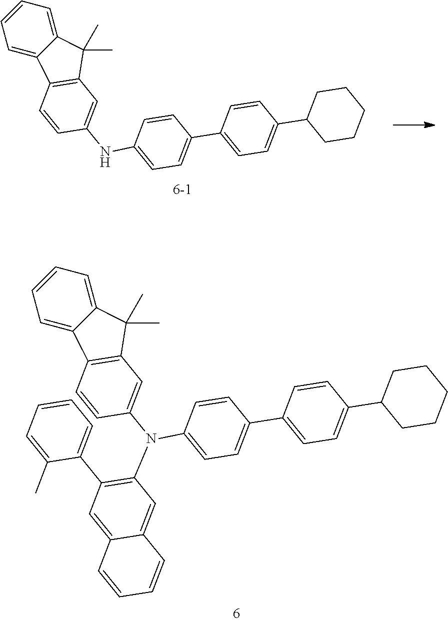 [SMILES: CC1(C)c2ccccc2-c2ccc(Nc3ccc(-c4ccc(C5CCCCC5)cc4)cc3)cc21 ; Cc1ccccc1-c1cc2ccccc2cc1N(c1ccc(-c2ccc(C3CCCCC3)cc2)cc1)c1ccc2c(c1)C(C)(C)c1ccccc1-2]

Compound 6-1 (10 mmol, 1 eq), Intermediate C1 (11 mmol, 1.1 eq), Pd₂(dba)₃ (0.5 mmol, 0.05 eq), t-BuONa (20 mmol, 2 eq), t-Bu₃P (1 mmol, 0.1 eq), and toluene (150 mL) were put in a 1-neck-round flask and stirred for 1 hour at 110° C. After completion of the reaction, the reaction product was subjected to column separation with MC/Hx=1/7 after working up with ether/H₂O to obtain 7.6 mmol of Compound 6 (yield: 76%).

### Synthesis Example 6: Synthesis of Compound 9

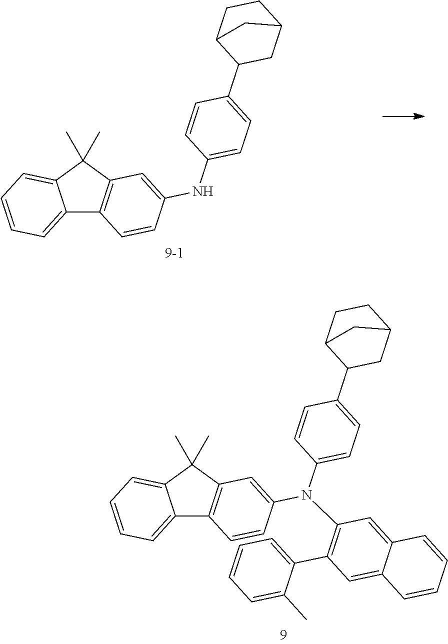 [SMILES: CC1(C)c2ccccc2-c2ccc(Nc3ccc(C4CC5CCC4C5)cc3)cc21 ; Cc1ccccc1-c1cc2ccccc2cc1N(c1ccc(C2CC3CCC2C3)cc1)c1ccc2c(c1)C(C)(C)c1ccccc1-2]

Compound 9-1 (10 mmol, 1 eq), Intermediate C1 (11 mmol, 1.1 eq), Pd₂(dba)₃ (0.5 mmol, 0.05 eq), t-BuONa (20 mmol, 2 eq), t-Bu₃P (1 mmol, 0.1 eq), and toluene (150 mL) were put in a 1-neck-round flask and stirred for 1 hour at 110° C. After completion of the reaction, the reaction product was subjected to column separation with MC/Hx=1/7 after working up with ether/H₂O to obtain 7.6 mmol of Compound 9 (yield: 76%).

### Synthesis Example 7: Synthesis of Compound 12

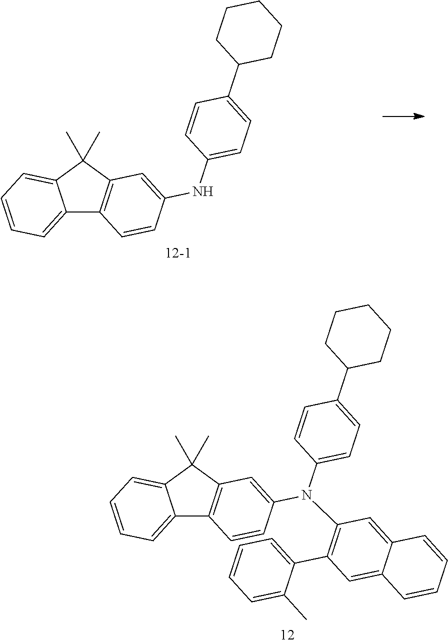 [SMILES: CC1(C)c2ccccc2-c2ccc(Nc3ccc(C4CCCCC4)cc3)cc21 ; Cc1ccccc1-c1cc2ccccc2cc1N(c1ccc(C2CCCCC2)cc1)c1ccc2c(c1)C(C)(C)c1ccccc1-2]

Compound 12-1 (10 mmol, 1 eq), Intermediate C1 (11 mmol, 1.1 eq), Pd₂(dba)₃ (0.5 mmol, 0.05 eq), t-BuONa (20 mmol, 2 eq), t-Bu₃P (1 mmol, 0.1 eq), and toluene (150 mL) were put in a 1-neck-round flask and stirred for 1 hour at 110° C. After completion of the reaction, the reaction product was subjected to column separation with MC/Hx=1/7 after working up with ether/H₂O to obtain 7.6 mmol of Compound 12 (yield: 76%).

### Synthesis Example 8: Synthesis of Compound 41

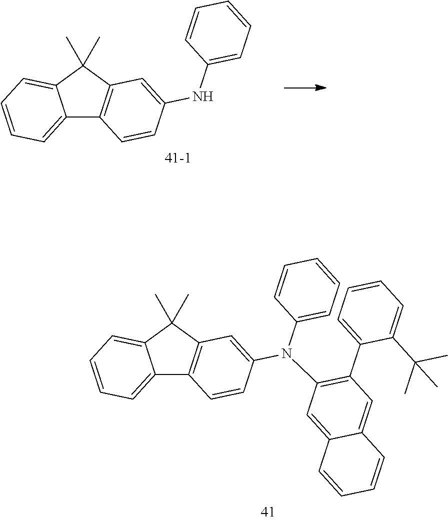 [SMILES: CC1(C)c2ccccc2-c2ccc(Nc3ccccc3)cc21 ; CC(C)(C)c1ccccc1-c1cc2ccccc2cc1N(c1ccccc1)c1ccc2c(c1)C(C)(C)c1ccccc1-2]

Compound 41-1 (10 mmol, 1 eq), Intermediate C2 (11 mmol, 1.1 eq), Pd₂(dba)₃ (0.5 mmol, 0.05 eq), t-BuONa (20 mmol, 2 eq), t-Bu₃P (1 mmol, 0.1 eq), and toluene (150 mL) were put in a 1-neck-round flask and stirred for 1 hour at 110° C. After completion of the reaction, the reaction product was subjected to column separation with MC/Hx=1/7 after working up with ether/H₂O to obtain 7.6 mmol of Compound 41 (yield: 76%).

### Synthesis Example 9: Synthesis of Compound 42

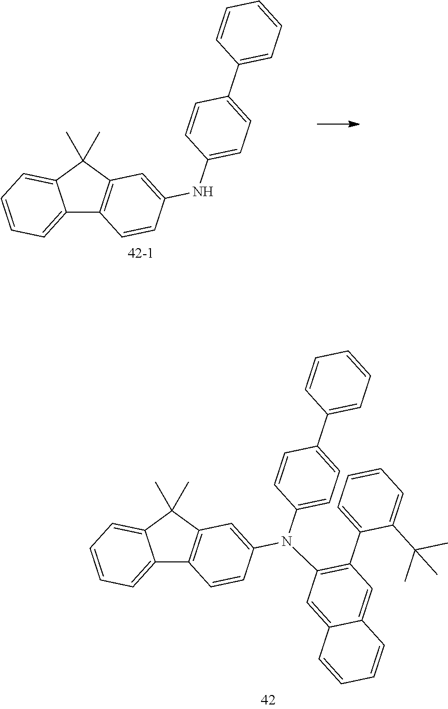 [SMILES: CC1(C)c2ccccc2-c2ccc(Nc3ccc(-c4ccccc4)cc3)cc21 ; CC(C)(C)c1ccccc1-c1cc2ccccc2cc1N(c1ccc(-c2ccccc2)cc1)c1ccc2c(c1)C(C)(C)c1ccccc1-2]

Compound 42-1 (10 mmol, 1 eq), Intermediate C2 (11 mmol, 1.1 eq), Pd₂(dba)₃ (0.5 mmol, 0.05 eq), t-BuONa (20 mmol, 2 eq), t-Bu₃P (1 mmol, 0.1 eq), and toluene (150 mL) were put in a 1-neck-round flask and stirred for 1 hour at 110° C. After completion of the reaction, the reaction product was subjected to column separation with MC/Hx=1/7 after working up with ether/H₂O to obtain 7.6 mmol of Compound 42 (yield: 76%).

### Synthesis Example 10: Synthesis of Compound 61

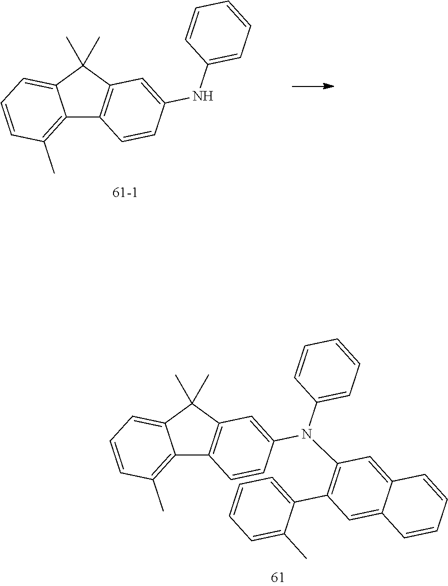 [SMILES: Cc1cccc2c1-c1ccc(Nc3ccccc3)cc1C2(C)C ; Cc1ccccc1-c1cc2ccccc2cc1N(c1ccccc1)c1ccc2c(c1)C(C)(C)c1cccc(C)c1-2]

61-1 (10 mmol, 1 eq), Intermediate C1 (11 mmol, 1.1 eq), Pd₂(dba)₃ (0.5 mmol, 0.05 eq), t-BuONa (20 mmol, 2 eq), t-Bu₃P (1 mmol, 0.1 eq), and toluene (150 mL) were put in a 1-neck-round flask and stirred for 1 hour at 110° C. After completion of the reaction, the reaction product was subjected to column separation with MC/Hx=1/7 after working up with ether/H₂O to obtain 7.6 mmol of Compound 61 (yield: 76%).

### Synthesis Example 11: Synthesis of Compound 62

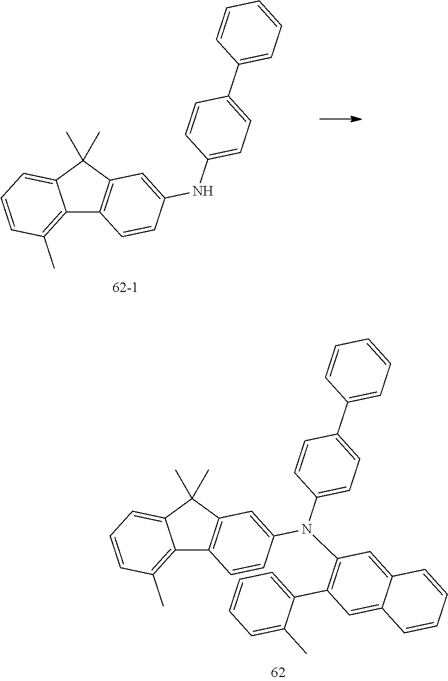 [SMILES: Cc1cccc2c1-c1ccc(Nc3ccc(-c4ccccc4)cc3)cc1C2(C)C ; Cc1ccccc1-c1cc2ccccc2cc1N(c1ccc(-c2ccccc2)cc1)c1ccc2c(c1)C(C)(C)c1cccc(C)c1-2]

Compound 62-1 (10 mmol, 1 eq), Intermediate C1 (11 mmol, 1.1 eq), Pd₂(dba)₃ (0.5 mmol, 0.05 eq), t-BuONa (20 mmol, 2 eq), t-Bu₃P (1 mmol, 0.1 eq), and toluene (150 mL) were put in a 1-neck-round flask and stirred for 1 hour at 110° C. After completion of the reaction, the reaction product was subjected to column separation with MC/Hx=1/7 after working up with ether/H₂O to obtain 7.6 mmol of Compound 62 (yield: 76%).

### Synthesis Example 12: Synthesis of Compound 72

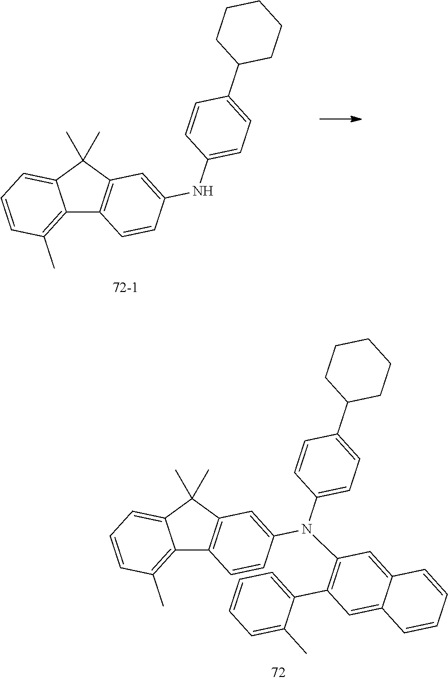 [SMILES: Cc1cccc2c1-c1ccc(Nc3ccc(C4CCCCC4)cc3)cc1C2(C)C ; Cc1ccccc1-c1cc2ccccc2cc1N(c1ccc(C2CCCCC2)cc1)c1ccc2c(c1)C(C)(C)c1cccc(C)c1-2]

Compound 72-1 (10 mmol, 1 eq), Intermediate C1 (11 mmol, 1.1 eq), Pd₂(dba)₃ (0.5 mmol, 0.05 eq), t-BuONa (20 mmol, 2 eq), t-Bu₃P (1 mmol, 0.1 eq), and toluene (150 mL) were put in a 1-neck-round flask and stirred for 1 hour at 110° C. After completion of the reaction, the reaction product was subjected to column separation with MC/Hx=1/7 after working up with ether/H₂O to obtain 7.6 mmol of Compound 72 (yield: 76%).

### Synthesis Example 13: Synthesis of Compound 301

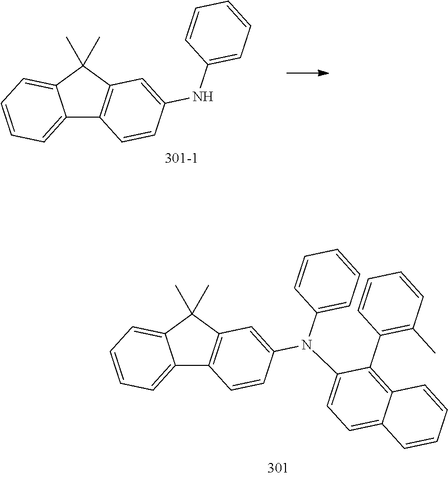 [SMILES: CC1(C)c2ccccc2-c2ccc(Nc3ccccc3)cc21 ; Cc1ccccc1-c1c(N(c2ccccc2)c2ccc3c(c2)C(C)(C)c2ccccc2-3)ccc2ccccc12]

Compound 301-1 (10 mmol, 1 eq), Intermediate C3 (11 mmol, 1.1 eq), Pd₂(dba)₃ (0.5 mmol, 0.05 eq), t-BuONa (20 mmol, 2 eq), t-Bu₃P (1 mmol, 0.1 eq), and toluene (150 mL) were put in a 1-neck-round flask and stirred for 1 hour at 110° C. After completion of the reaction, the reaction product was subjected to column separation with MC/Hx=1/7 after working up with ether/H₂O to obtain 7.6 mmol of Compound 301 (yield: 76%).

### Synthesis Example 14: Synthesis of Compound 302

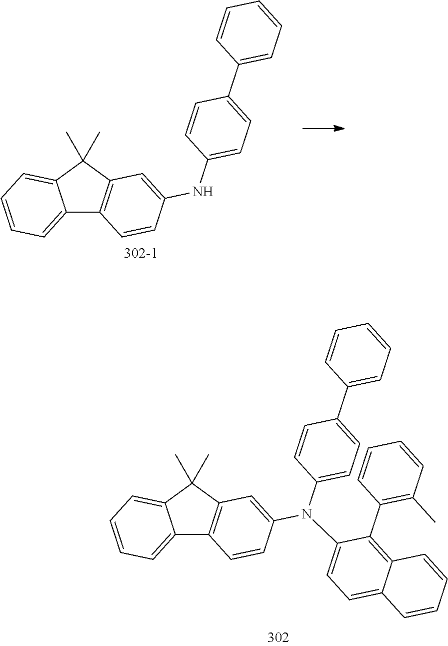 [SMILES: CC1(C)c2ccccc2-c2ccc(Nc3ccc(-c4ccccc4)cc3)cc21 ; Cc1ccccc1-c1c(N(c2ccc(-c3ccccc3)cc2)c2ccc3c(c2)C(C)(C)c2ccccc2-3)ccc2ccccc12]

Compound 302-1 (10 mmol, 1 eq), Intermediate C3 (11 mmol, 1.1 eq), Pd₂(dba)₃ (0.5 mmol, 0.05 eq), t-BuONa (20 mmol, 2 eq), t-Bu₃P (1 mmol, 0.1 eq), and toluene (150 mL) were put in a 1-neck-round flask and stirred for 1 hour at 110° C. After completion of the reaction, the reaction product was subjected to column separation with MC/Hx=1/7 after working up with ether/H₂O to obtain 7.6 mmol of Compound 302 (yield: 76%).

### Synthesis Example 15: Synthesis of Compound 329

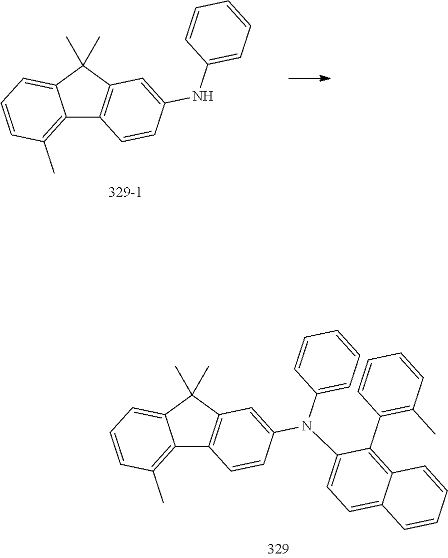 [SMILES: Cc1cccc2c1-c1ccc(Nc3ccccc3)cc1C2(C)C ; Cc1ccccc1-c1c(N(c2ccccc2)c2ccc3c(c2)C(C)(C)c2cccc(C)c2-3)ccc2ccccc12]

Compound 329-1 (10 mmol, 1 eq), Intermediate C3 (11 mmol, 1.1 eq), Pd₂(dba)₃ (0.5 mmol, 0.05 eq), t-BuONa (20 mmol, 2 eq), t-Bu₃P (1 mmol, 0.1 eq), and toluene (150 mL) were put in a 1-neck-round flask and stirred for 1 hour at 110° C. After completion of the reaction, the reaction product was subjected to column separation with MC/Hx=1/7 after working up with ether/H₂O to obtain 7.6 mmol of Compound 329 (yield: 76%).

### Synthesis Example 16: Synthesis of Compound 330

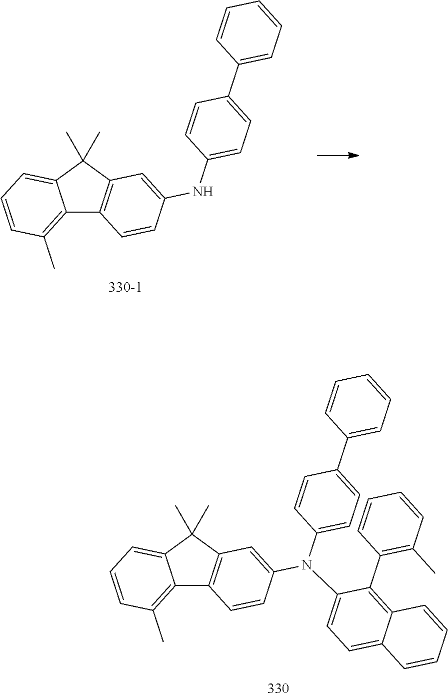 [SMILES: Cc1cccc2c1-c1ccc(Nc3ccc(-c4ccccc4)cc3)cc1C2(C)C ; Cc1ccccc1-c1c(N(c2ccc(-c3ccccc3)cc2)c2ccc3c(c2)C(C)(C)c2cccc(C)c2-3)ccc2ccccc12]

Compound 330-1 (10 mmol, 1 eq), Intermediate C3 (11 mmol, 1.1 eq), Pd₂(dba)₃ (0.5 mmol, 0.05 eq), t-BuONa (20 mmol, 2 eq), t-Bu₃P (1 mmol, 0.1 eq), and toluene (150 mL) were put in a 1-neck-round flask and stirred for 1 hour at 110° C. After completion of the reaction, the reaction product was subjected to column separation with MC/Hx=1/7 after working up with ether/H₂O to obtain 7.6 mmol of Compound 330 (yield: 76%).

### Synthesis Example 17: Synthesis of Compound 337

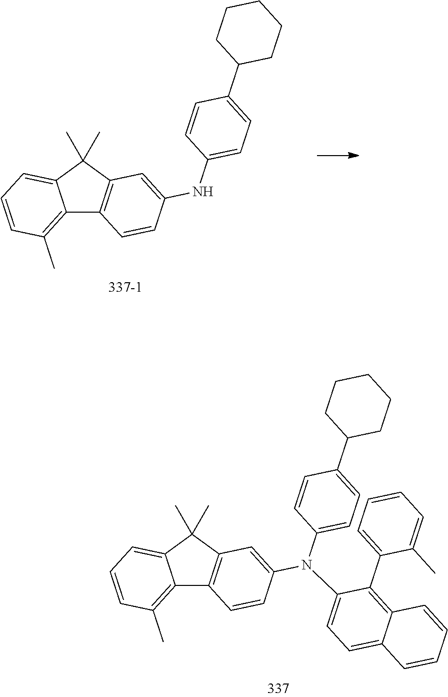 [SMILES: Cc1cccc2c1-c1ccc(Nc3ccc(C4CCCCC4)cc3)cc1C2(C)C ; Cc1ccccc1-c1c(N(c2ccc(C3CCCCC3)cc2)c2ccc3c(c2)C(C)(C)c2cccc(C)c2-3)ccc2ccccc12]

Compound 337-1 (10 mmol, 1 eq), Intermediate C3 (11 mmol, 1.1 eq), Pd₂(dba)₃ (0.5 mmol, 0.05 eq), t-BuONa (20 mmol, 2 eq), t-Bu₃P (1 mmol, 0.1 eq), and toluene (150 mL) were put in a 1-neck-round flask and stirred for 1 hour at 110° C. After completion of the reaction, the reaction product was subjected to column separation with MC/Hx=1/7 after working up with ether/H₂O to obtain 7.6 mmol of Compound 337 (yield: 76%).

### Synthesis Example 18: Synthesis of Compound 385

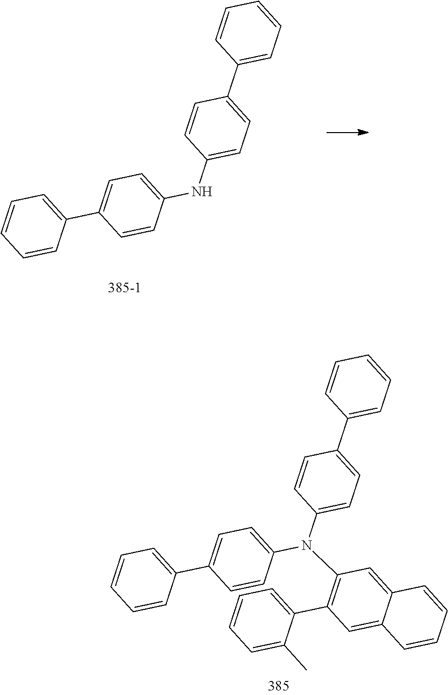 [SMILES: c1ccc(-c2ccc(Nc3ccc(-c4ccccc4)cc3)cc2)cc1 ; Cc1ccccc1-c1cc2ccccc2cc1N(c1ccc(-c2ccccc2)cc1)c1ccc(-c2ccccc2)cc1]

Compound 385-1 (10 mmol, 1 eq), Intermediate C1 (11 mmol, 1.1 eq), Pd₂(dba)₃ (0.5 mmol, 0.05 eq), t-BuONa (20 mmol, 2 eq), t-Bu₃P (1 mmol, 0.1 eq), and toluene (150 mL) were put in a 1-neck-round flask and stirred for 1 hour at 110° C. After completion of the reaction, the reaction product was subjected to column separation with MC/Hx=1/7 after working up with ether/H₂O to obtain 7.6 mmol of Compound 385 (yield: 76%).

Results of measuring ¹H NMR and high-resolution mass (HR-MS) of compounds synthesized in Synthesis Examples 1 to 18 and Comparative Synthesis Example 1 are shown in Table 1. Synthesis methods of other compounds in addition to the compounds synthesized in Synthesis Examples 1 to 18 may be easily recognized by those skilled in the art by referring to the synthesis paths and source materials.

### Comparative Example Compound 1

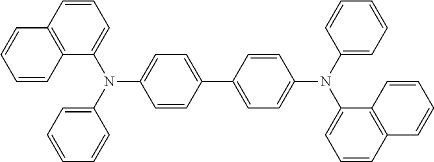 [SMILES: c1ccc(N(c2ccc(-c3ccc(N(c4ccccc4)c4cccc5ccccc45)cc3)cc2)c2cccc3ccccc23)cc1]

TABLE 1

| Synthesis or | | | |
|---|---|---|---|
| Comparative | | HR-MS (m/z) [M⁺] | |
| Example ID | ¹H NMR (CDCl₃, 500 MHz) | found | calc. |
| Synthesis | 7.90-7.72 (m, 6H), 7.38-7.00 (m, | 501.45 | 501.24 |
| Example 1 | 16H), 2.23 (s, 3H), 1.69 (s, 6H) | | |
| Synthesis | 7.90-7.72 (m, 6H), 7.38-7.00 (m, | 577.34 | 577.27 |
| Example 2 | 20H), 2.23 (s, 3H), 1.69 (s, 6H) | | |
| Synthesis | 7.90-7.72 (m, 6H), 7.38-7.00 (m, | 577.40 | 577.27 |
| Example 3 | 20H), 2.23 (s, 3H), 1.69 (s, 6H) | | |
| Synthesis | 8.06 (d, 2H), 7.90-7.72 (m, 9H), | 729.45 | 729.34 |
| Example 4 | 7.38-7.00 (m, 23H), 2.23 (s, 3H), | | |
| | 1.69 (s, 6H) | | |
| Synthesis | 7.90-7.72 (m, 6H), 7.38-7.00 (m, | 659.40 | 659.35 |
| Example 5 | 25H), 2.23 (s, 3H), 1.69 (s, 6H), | | |
| | 1.53-1.43 (m, 5H) | | |
| Synthesis | 7.90-7.72 (m, 6H), 7.38-7.00 (m, | 595.37 | 595.32 |
| Example 6 | 15H), 2.23 (s, 3H), 1.69 (s, 6H), | | |
| | 1.82-1.31 (m, 11H) | | |
| Synthesis | 7.90-7.72 (m, 6H), 7.38-7.00 (m, | 583.85 | 583.32 |
| Example 7 | 15H), 2.23 (s, 3H), 1.69 (s, 6H), | | |
| | 1.82-1.31 (m, 11H) | | |
| Synthesis | 7.90-7.72 (m, 6H), 7.38-7.00 (m, | 543.62 | 543.29 |
| Example 8 | 16H), 1.69 (s, 6H), 1.35 (s, 9H) | | |
| Synthesis | 7.90-7.72 (m, 6H), 7.38-7.00 (m, | 619.53 | 619.32 |
| Example 9 | 20H), 1.69 (s, 6H), 1.35 (s, 9H) | | |
| Synthesis | 7.90-7.72 (m, 5H), 7.55 (d, 2H), | 515.65 | 515.26 |
| Example 10 | 7.38-7.00 (m, 14H), 2.57 (s, 3H), | | |
| | 2.23 (s, 3H), 1.69 (s, 6H) | | |
| Synthesis | 7.90-7.72 (m, 5H), 7.55 (d, 2H), | 591.72 | 591.29 |
| Example 11 | 7.38-7.00 (m, 14H), 2.57 (s, 3H), | | |
| | 2.23 (s, 3H), 1.69 (s, 6H) | | |
| Synthesis | 7.90-7.72 (m, 6H), 7.38-7.00 (m, | 597.86 | 597.34 |
| Example 12 | 14H), 2.23 (s, 3H), 2.57 (s, 3H), | | |
| | 1.69 (s, 6H), 1.82-1.31 (m, 11H) | | |
| Synthesis | 7.90-7.72 (m, 6H), 7.38-7.00 (m, | 501.62 | 501.24 |
| Example 13 | 16H), 2.23 (s, 3H), 1.69 (s, 6H) | | |
| Synthesis | 7.90-7.72 (m, 6H), 7.38-7.00 (m, | 577.75 | 577.27 |
| Example 14 | 20H), 2.23 (s, 3H), 1.69 (s, 6H) | | |
| Synthesis | 7.90-7.72 (m, 6H), 7.38-7.00 (m, | 515.43 | 515.26 |
| Example 15 | 15H), 2.57 (s, 3H), 2.23 (s, 3H), | | |
| | 1.69 (s, 6H) | | |
| Synthesis | 7.90-7.72 (m, 6H), 7.38-7.00 (m, | 607.83 | 607.32 |
| Example 16 | 23H), 2.57 (s, 3H), 2.23 (s, 3H), | | |
| | 1.69 (s, 6H) | | |
| Synthesis | 7.90-7.72 (m, 6H), 7.38-7.00 (m, | 597.94 | 597.34 |
| Example 17 | 14H), 2.23 (s, 3H), 2.57 (s, 3H), | | |
| | 1.69 (s, 6H), 1.82-1.31 (m, 11H) | | |
| Synthesis | 7.90-7.72 (m, 7H), 7.38-7.00 (m, | 537.68 | 537.24 |
| Example 18 | 21H), 2.23 (s, 3H) | | |
| Comparative | 8.22-8.15 (m, 4H), 7.81 (d, 2H), | 585.62 | 585.26 |
| Example 1 | 7.63-7.49 (m, 12H), 7.37 (d, 4H), | | |
| | 7.24 (d, 4H), 7.08-7.00 (m, 6H) | | |

### Evaluation Example 1

The LUMO and HOMO values of the compounds synthesized in Synthesis Examples 1 to 18 and Comparative Example 1 were measured according to the methods described in Table 2, and the results thereof are shown in Table 3.

TABLE 2

| | |
|---|---|
| HOMO | By utilizing cyclic voltammetry (CV) (electrolyte: 0.1M |
| energy | Bu₄NPF₆/solvent: dimethylforamide (DMF)/electrode: 3- |
| level | electrode system (working electrode: GC, reference |
| evaluation | electrode: Ag/AgCl, and auxiliary electrode: Pt)), the potential |
| method | (V)-current (A) graph of each compound was obtained, and |
| | then, from the oxidation onset of the graph, the HOMO |
| | energy level of each compound was calculated. |
| LUMO | By utilizing cyclic voltammetry (CV) (electrolyte: 0.1M |
| energy | Bu₄NPF₆/solvent: dimethylforamide (DMF)/electrode: 3- |
| level | electrode system (working electrode: GC, reference |
| evaluation | electrode: Ag/AgCl, and auxiliary electrode: Pt)), the potential |
| method | (V)-current (A) graph of each compound was obtained, and |
| | then, from the reduction onset of the graph, the LUMO |
| | energy level of each compound was calculated. |

TABLE 3

| Synthesis or Comparative | | |
|---|---|---|
| Example ID | HOMO (eV) | LUMO (eV) |
| Synthesis Example 1 | −5.18 | −1.85 |
| Synthesis Example 2 | −5.16 | −1.85 |
| Synthesis Example 3 | −5.20 | −1.90 |
| Synthesis Example 4 | −5.13 | −1.95 |
| Synthesis Example 5 | −5.19 | −1.93 |
| Synthesis Example 6 | −5.12 | −1.88 |
| Synthesis Example 7 | −5.13 | −1.90 |
| Synthesis Example 8 | −5.10 | −1.84 |
| Synthesis Example 9 | −5.19 | −1.85 |
| Synthesis Example 10 | −5.17 | −1.86 |
| Synthesis Example 11 | −5.20 | −1.82 |
| Synthesis Example 12 | −5.14 | −1.85 |
| Synthesis Example 13 | −5.17 | −1.84 |
| Synthesis Example 14 | −5.16 | −1.86 |
| Synthesis Example 15 | −5.16 | −1.92 |
| Synthesis Example 16 | −5.18 | −1.93 |
| Synthesis Example 17 | −5.14 | −1.87 |
| Synthesis Example 18 | −5.13 | −1.86 |
| Comparative Example 1 | −5.12 | −1.85 |

### Example 1

As an anode, a glass substrate (product of Corning Inc.) with a 15 Ω/cm² (ohms per square centimeter) (1,200 Å) layer of ITO formed thereon was cut to a size of 50 mm×50 mm×0.7 mm, sonicated utilizing isopropyl alcohol and pure water each for 5 minutes, washed by irradiation of ultraviolet rays and exposure of ozone thereto for 30 minutes, and then mounted on a vacuum deposition apparatus.

A 2-TNATA was vacuum-deposited on the anode to form a hole injection layer having a thickness of 600 Å (angstroms), and Compound 1 was vacuum-deposited on the hole injection layer to form a hole transport layer having a thickness of 300 Å.

9,10-di(naphthalene-2-yl)anthracene (DNA) as a host material and 4,4′-bis[2-(4-(N,N-diphenylamino)phenyl)vinyl]biphenyl(DPAVBi) as a dopant material were concurrently (e.g., simultaneously) vacuum-deposited on the hole transport layer at a weight ratio of 98:2 to form an emission layer having a thickness of 300 Å.

Subsequently, Alq3 was vacuum-deposited on the emission layer to form an electron transport layer having a thickness of 300 Å, LiF was vacuum-deposited on the electron transport layer to form an electron injection layer having a thickness of 10 Å, and Al was vacuum-deposited thereon to form a cathode having a thickness of 3,000 Å, thereby completing the manufacture of an organic light-emitting device.

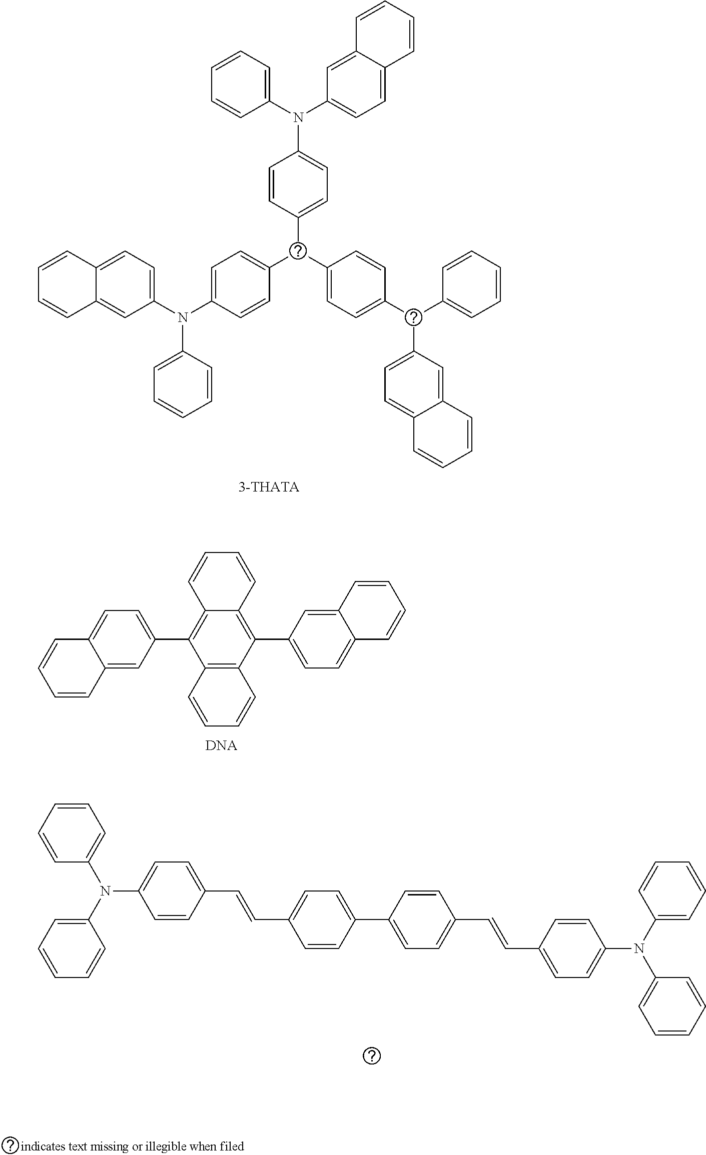

### Examples 2 to 18 and Comparative Examples 1 to 5

The organic light-emitting devices of Examples 2 to 18 and Comparative Examples 1 to 5 were manufactured in substantially the same manner as in Example 1, except that, when forming the hole transport layer, Compounds 2, 4, 5, 6, 9, 12, 41, 42, 61, 62, 72, 301, 302, 329, 330, 337, 385, and the compounds of Comparative Example Compounds 1 to 5 were utilized instead of Compound 1 in Example 1.

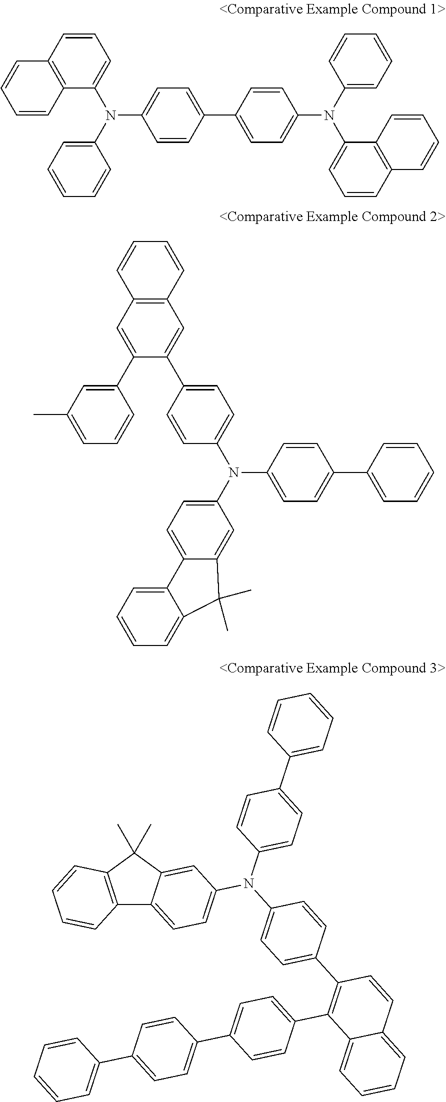 [SMILES: c1ccc(N(c2ccc(-c3ccc(N(c4ccccc4)c4cccc5ccccc45)cc3)cc2)c2cccc3ccccc23)cc1 ; Cc1cccc(-c2cc3ccccc3cc2-c2ccc(N(c3ccc(-c4ccccc4)cc3)c3ccc4c(c3)C(C)(C)c3ccccc3-4)cc2)c1 ; CC1(C)c2ccccc2-c2ccc(N(c3ccc(-c4ccccc4)cc3)c3ccc(-c4ccc5ccccc5c4-c4ccc(-c5ccc(-c6ccccc6)cc5)cc4)cc3)cc21]

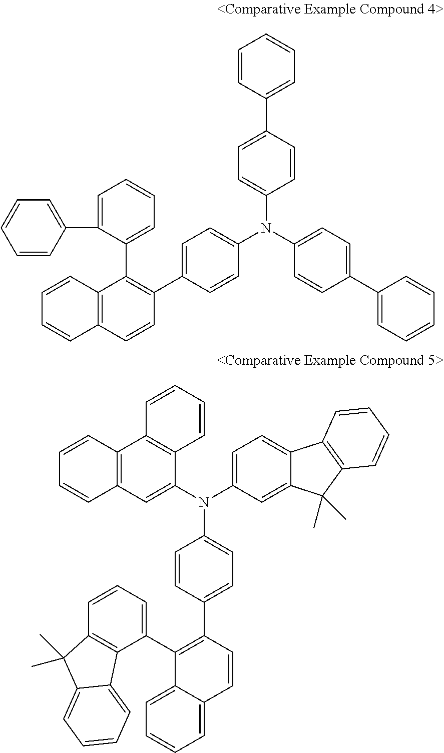 [SMILES: c1ccc(-c2ccc(N(c3ccc(-c4ccccc4)cc3)c3ccc(-c4ccc5ccccc5c4-c4ccccc4-c4ccccc4)cc3)cc2)cc1 ; CC1(C)c2ccccc2-c2ccc(N(c3ccc(-c4ccc5ccccc5c4-c4cccc5c4-c4ccccc4C5(C)C)cc3)c3cc4ccccc4c4ccccc34)cc21]

### Evaluation Example 2

The operating characteristics of the organic light-emitting devices manufactured according to Examples 1 to 18 and Comparative Examples 1 to 5 were determined by utilizing Keithley MU 236 and luminance meter PR650. The driving voltage (V) was measured at 1000 candela per square meter (cd/m²), current density was measured in milliampere per square centimeter (mA/cm²), luminance was measured in candela per square meter (cd/m²), luminescence efficiency was measured in candela per ampere (cd/A), and lifespan measured in hours (h). The half lifespan was reported at 100 mA/cm². The results are shown in Table 4.

TABLE 4

| | | | | | | | Half |
|---|---|---|---|---|---|---|---|
| | Hole | Driving | Current | | | | lifespan |
| | transport | voltage | density | Luminance | Efficiency | Emission | (hr @ 100 |
| ID | material | (V) | (mA/cm²) | (cd/m²) | (cd/A) | color | mA/cm²) |
| Example 1 | Compound 1 | 5.13 | 50 | 3200 | 6.40 | Blue | 650 |
| Example 2 | Compound 2 | 5.08 | 50 | 3150 | 6.30 | Blue | 620 |
| Example 3 | Compound 4 | 4.99 | 50 | 3100 | 6.20 | Blue | 580 |
| Example 4 | Compound 5 | 5.19 | 50 | 3140 | 6.28 | Blue | 530 |
| Example 5 | Compound 6 | 5.20 | 50 | 3270 | 6.54 | Blue | 480 |
| Example 6 | Compound 9 | 4.95 | 50 | 3190 | 6.38 | Blue | 530 |
| Example 7 | Compound 12 | 5.07 | 50 | 3155 | 6.31 | Blue | 580 |
| Example 8 | Compound 41 | 5.08 | 50 | 3200 | 6.40 | Blue | 540 |
| Example 9 | Compound 42 | 5.23 | 50 | 3115 | 6.23 | Blue | 570 |
| Example 10 | Compound 61 | 4.98 | 50 | 3200 | 6.40 | Blue | 620 |
| Example 11 | Compound 62 | 5.05 | 50 | 3245 | 6.49 | Blue | 650 |
| Example 12 | Compound 72 | 5.13 | 50 | 3200 | 6.40 | Blue | 650 |
| Example 13 | Compound 301 | 5.19 | 50 | 3140 | 6.28 | Blue | 530 |
| Example 14 | Compound 302 | 5.20 | 50 | 3270 | 6.54 | Blue | 480 |
| Example 15 | Compound 329 | 4.95 | 50 | 3190 | 6.38 | Blue | 530 |
| Example 16 | Compound 330 | 5.07 | 50 | 3155 | 6.31 | Blue | 580 |
| Example 17 | Compound 337 | 5.08 | 50 | 3200 | 6.40 | Blue | 540 |
| Example 18 | Compound 385 | 5.70 | 50 | 2605 | 5.51 | Blue | 300 |
| Comparative | Comparative | 7.01 | 50 | 2645 | 5.29 | Blue | 258 |
| Example 1 | Example | | | | | | |
| | Compound 1 | | | | | | |
| | (NPB) | | | | | | |
| Comparative | Comparative | 7.53 | 50 | 2305 | 5.86 | Blue | 220 |
| Example 2 | Example | | | | | | |
| | Compound 2 | | | | | | |
| Comparative | Comparative | 8.54 | 50 | 2285 | 4.78 | Blue | 180 |
| Example 3 | Example | | | | | | |
| | Compound 3 | | | | | | |
| Comparative | Comparative | 7.98 | 50 | 2400 | 5.30 | Blue | 280 |
| Example 4 | Example | | | | | | |
| | Compound 4 | | | | | | |
| Comparative | Comparative | 8.20 | 50 | 2555 | 4.88 | Blue | 260 |
| Example 5 | Example | | | | | | |
| | Compound 5 | | | | | | |

According to one or more embodiments, utilizing the amine-containing compound may enable the manufacture of a light-emitting device having high efficiency and a long lifespan and a high-quality electronic apparatus including the light-emitting device.

It should be understood that the practical example embodiments of the present disclosure should be considered in a descriptive sense only and not for purposes of limitation. Descriptions of features or aspects within each embodiment should typically be considered as available for other similar features or aspects in other embodiments. While one or more embodiments have been described with reference to the drawings, it will be understood by those of ordinary skill in the art that one or more suitable changes in form and details may be made therein without departing from the spirit and scope as defined by the following claims and equivalents thereof.

What is claimed is:

1. A light-emitting device comprising:
a first electrode;
a second electrode facing the first electrode;
an interlayer located between the first electrode and the second electrode, the interlayer comprising an emission layer; and
an amine-containing compound represented by Formula 1 or 2: 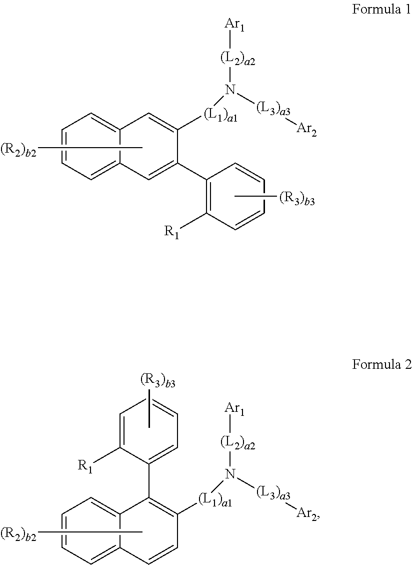
and
wherein, in Formulae 1 and 2,
L₁, L₂, and L₃ are each independently a C₃-C₆₀ carbocyclic group unsubstituted or substituted with at least one R₁₀ₐ or a C₁-C₆₀ heterocyclic group unsubstituted or substituted with at least one R₁₀ₐ,
Ar₁ and Ar₂ are each independently a C₆-C₆₀ aryl group unsubstituted or substituted with at least one R₁₀ₐ or a C₁-C₆₀ heteroaryl group unsubstituted or substituted with at least one R₁₀ₐ,
R₁ is deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
R₂ and R₃ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
a1, a2, a3, b2, and b3 are each independently an integer from 0 to 6,
when b3 is 1 or more and R₃ is not hydrogen, R₁ and R₃ are not linked to each other,
each R₁₀ₐ independently is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each being unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or a combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each being unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or a combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
wherein Q₁, Q₂, Q₃, Q₁₁, Q₁₂, Q₁₃, Q₂₁, Q₂₂, Q₂₃, Q₃₁, Q₃₂, and Q₃₃ are each independently: hydrogen; deuterium; —F; —Cl; —Br; —I; a hydroxyl group; a cyano group; a nitro group; or a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, or a C₁-C₆₀ heterocyclic group, each being unsubstituted or substituted with deuterium, —F, a cyano group, a C₁-C₆₀ alkyl group, a C₁-C₆₀ alkoxy group, a phenyl group, a biphenyl group, or a combination thereof.

2. The light-emitting device of claim 1, wherein
the first electrode is an anode,
the second electrode is a cathode,
the interlayer comprises a hole transport region between the first electrode and the emission layer and an electron transport region between the emission layer and the second electrode,
the hole transport region comprises a hole injection layer, a hole transport layer, an emission auxiliary layer, an electron blocking layer, or a combination thereof, and
the electron transport region comprises a hole blocking layer, an electron transport layer, an electron injection layer, an electron control layer, or a combination thereof.

3. The light-emitting device of claim 1, wherein the interlayer comprises the amine-containing compound.

4. The light-emitting device of claim 2, wherein the hole transport region comprises the amine-containing compound.

5. The light-emitting device of claim 2, wherein
the hole transport layer comprises the amine-containing compound, and
the hole transport layer is in direct contact with the emission layer.

6. The light-emitting device of claim 2,
wherein the light-emitting device further comprises a capping layer external to the first electrode, and
wherein the capping layer comprises the amine-containing compound.

7. An electronic apparatus comprising the light-emitting device of claim 1.

8. The electronic apparatus of claim 7, further comprising a color filter, a color conversion layer, a touch screen layer, a polarizing layer, or a combination thereof.

9. An electronic equipment comprising the light-emitting device of claim 1.

10. The electronic equipment of claim 9, wherein the electronic equipment is a flat panel display, a curved display, a computer monitor, a medical monitor, a television, a billboard, an indoor or outdoor light and/or light for signal, a head-up display, a fully or partially transparent display, a flexible display, a rollable display, a foldable display, a stretchable display, a laser printer, a telephone, a portable phone, a tablet personal computer, a phablet, a personal digital assistant (PDA), a wearable device, a laptop computer, a digital camera, a camcorder, a viewfinder, a micro display, a three-dimensional (3D) display, a virtual reality or augmented reality display, a vehicle, a video wall with multiple displays tiled together, a theater or stadium screen, a phototherapy device, and/or a signboard.

11. An amine-containing compound represented by Formula 1 or 2: 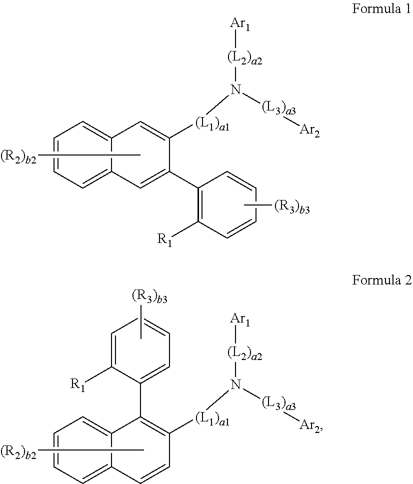
wherein, in Formulae 1 and 2,
L₁, L₂, and L₃ are each independently a C₃-C₆₀ carbocyclic group unsubstituted or substituted with at least one R₁₀ₐ or a C₁-C₆₀ heterocyclic group unsubstituted or substituted with at least one R₁₀ₐ,
Ar₁ and Ar₂ are each independently a C₆-C₆₀ aryl group unsubstituted or substituted with at least one R₁₀ₐ or a C₁-C₆₀ heteroaryl group unsubstituted or substituted with at least one R₁₀ₐ,
R₁ is deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
R₂ and R₃ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
a1, a2, a3, b2, and b3 are each independently an integer from 0 to 6,
when b3 is 1 or more and R₃ is not hydrogen, R₁ and R₃ are not linked to each other,
each R₁₀ₐ independently is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each being unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or a combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each being unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or a combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
wherein Q₁, Q₂, Q₃, Q₁₁, Q₁₂, Q₁₃, Q₂₁, Q₂₂, Q₂₃, Q₃₁, Q₃₂, and Q₃₃ are each independently: hydrogen; deuterium; —F; —Cl; —Br; —I; a hydroxyl group; a cyano group; a nitro group; or a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, or a C₁-C₆₀ heterocyclic group, each being unsubstituted or substituted with deuterium, —F, a cyano group, a C₁-C₆₀ alkyl group, a C₁-C₆₀ alkoxy group, a phenyl group, a biphenyl group, or a combination thereof.

12. The amine-containing compound of claim 11, wherein, in Formulae 1 and 2, groups represented by *-(L₂)ₐ₂-Ar₁ and *-(L₃)ₐ₃-Ar₂ are different from each other, and wherein “*” is a binding site to a neighboring atom.

13. The amine-containing compound of claim 11, wherein L₁, L₂, and L₃ are each independently a cyclopentane group, a cyclohexane group, a benzene group, a naphthalene group, an azulene group, an indacene group, an acenaphthylene group, a phenalene group, a phenanthrene group, an anthracene group, a pyrrole group, a thiophene group, a furan group, an indole group, a benzoindole group, a naphtho indole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, a pyrazole group, an imidazole group, a triazole group, a benzopyrazole group, a benzimidazole group, an indazole group, a pyridine group, a pyrimidine group, a pyrazine group, a pyridazine group, a triazine group, a quinoline group, an isoquinoline group, a benzoquinoline group, a benzoisoquinoline group, an imidazopyridine group, an imidazopyrimidine group, an imidazotriazine group, an imidazopyrazine group, an imidazopyridazine group, an azacarbazole group, an azadibenzosilole group, an azadibenzothiophene group, or an azadibenzofuran group, each unsubstituted or substituted with at least one deuterium, a C₁-C₂₀ alkyl group, a cyclohexane group, an adamantane group, a norbornane group, a benzene group, a naphthalene group, a phenanthrene group, a fluorene group, spiro-bifluorene group, a dibenzofuran group, a dibenzothiophene group, or a combination thereof.

14. The amine-containing compound of claim 11, wherein L₁, L₂, and L₃ are each independently represented by one of Formulae 3-1 to 3-12: 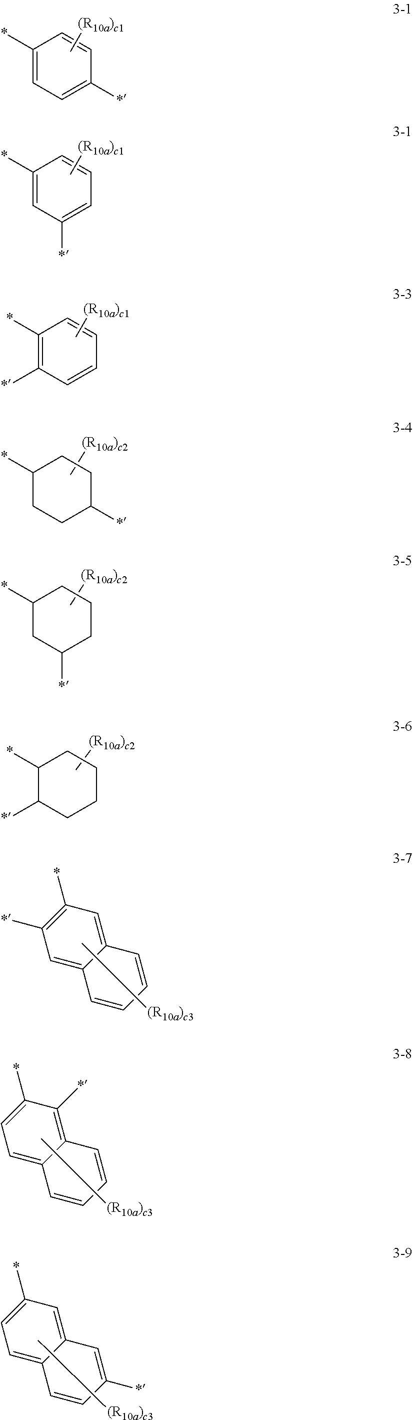 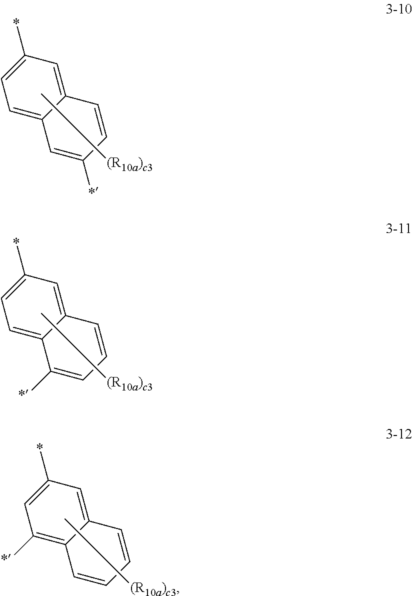
and
wherein, in Formulae 3-1 to 3-12,
R₁₀ₐ is as defined in in Formula 1 or 2,
c1 is an integer from 0 to 4,
c2 is an integer from 0 to 8,
c3 is an integer from 0 to 6, and
* and *′ are each independently a binding site to a neighboring atom.

15. The amine-containing of claim 11, wherein Ar₁ and Ar₂ are each independently a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, or a fluorene group, each unsubstituted or substituted with at least one deuterium, a C₁-C₂₀ alkyl group, a cyclohexane group, an adamantane group, a norbornane group, a benzene group, a naphthalene group, a phenanthrene group, a fluorene group, a spiro-bifluorene group, a dibenzofuran group, a dibenzothiophene group, or a combination thereof.

16. The amine-containing compound of claim 11, wherein Ar₁ and Ar₂ are each independently represented by one of Formulae 4-1 to 4-13: 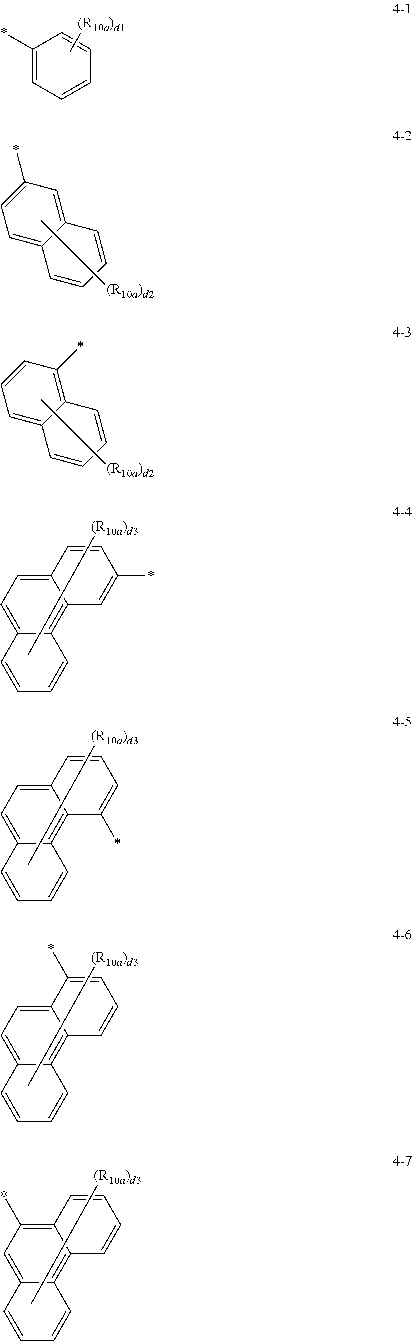 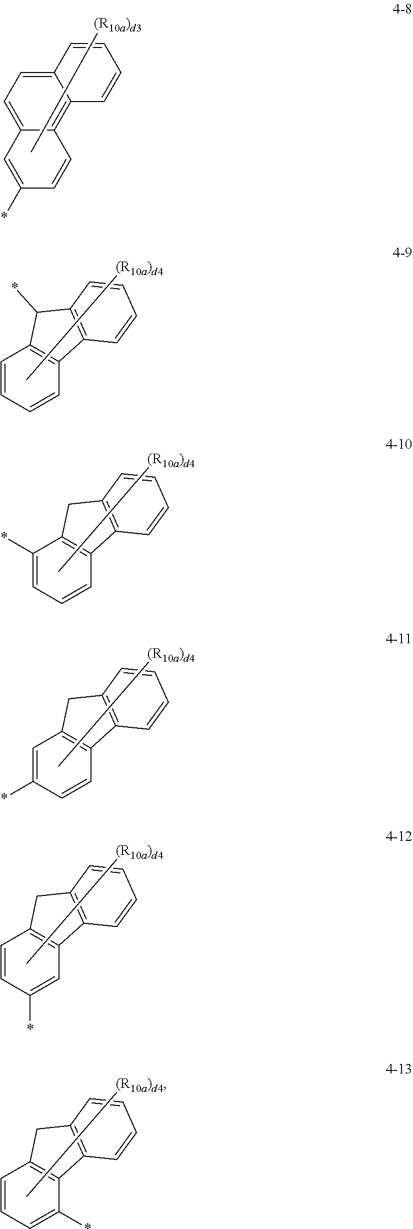
and
wherein, in Formulae 4-1 to 4-13,
R₁₀ₐ is as defined in in Formula 1 or 2,
d1 is an integer from 0 to 5,
d2 is an integer from 0 to 7,
d3 is an integer from 0 to 10,
d4 is an integer from 0 to 10, and
* indicates a binding site to a neighboring atom.

17. The amine-containing compound of claim 11, wherein R₁ is a methyl group, an ethyl group, an n-propyl group, an iso-propyl group, an n-butyl group, an isobutyl group, a sec-butyl group, a tert-butyl group, an n-pentyl group, an isopentyl group, a sec-pentyl group, a tert-pentyl group, a 2-methylbutyl group, a 2-2dimethylpropyl group, a 1-ethylpropyl group, or a 1,2-dimethylpropyl group, each unsubstituted or substituted with deuterium, —F, —Cl, —Br, —I, a hydroxyl group, a cyano group, or a nitro group.

18. The amine-containing compound of claim 11, wherein
R₁₀ₐ is:
a methyl group, an ethyl group, an n-propyl group, an isopropyl group, an n-butyl group, an isobutyl group, a sec-butyl group, a tert-butyl group, an n-pentyl group, an isopentyl group, a sec-pentyl group, a tert-pentyl group, a 2-methylbutyl group, a 2-2 dimethylpropyl group, a 1-ethylpropyl group, or a 1,2-dimethylpropyl group, each unsubstituted or substituted with deuterium, —F, a cyano group, or a combination thereof; or
a cyclohexane group, an adamantane group, a norbornane group, or a benzene group, each unsubstituted or substituted with a methyl group, an ethyl group, an n-propyl group, an isopropyl group, an n-butyl group, an isobutyl group, a sec-butyl group, a tert-butyl group, an n-pentyl group, an isopentyl group, a sec-pentyl group, a tert-pentyl group, a 2-methylbutyl group, a 2-2 dimethylpropyl group, a 1-ethylpropyl group, a 1,2-dimethylpropyl group, or a phenyl group, each unsubstituted or substituted with deuterium, —F, —Cl, —Br, —I, a hydroxyl group, a cyano group, or a nitro group.

19. The amine-containing compound of claim 11, wherein the amine-containing compound is represented by one of Formulae 1-1 to 1-9 and 2-1 to 2-9: 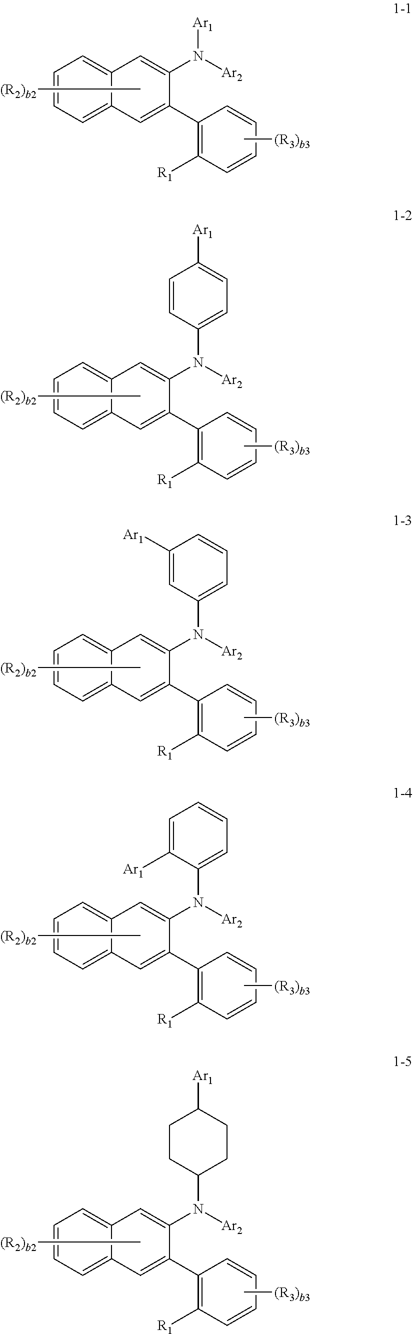 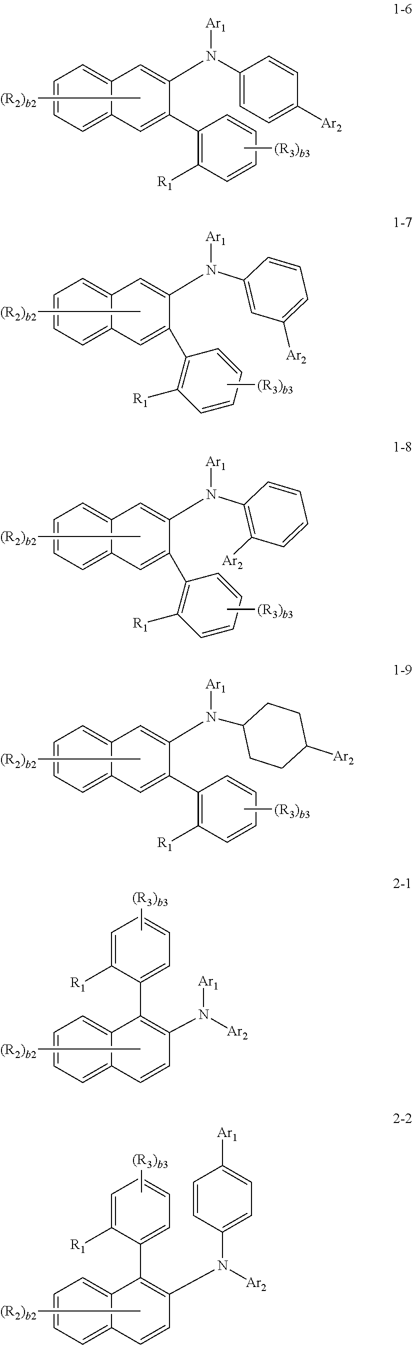 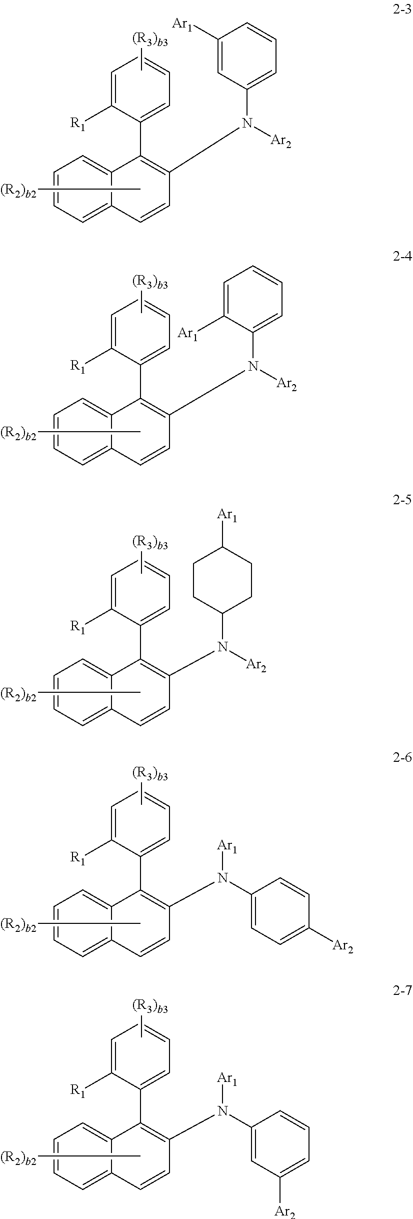 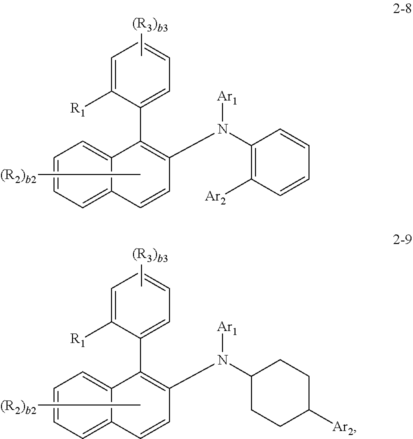
and
wherein, in Formulae 1-1 to 1-9 and 2-1 to 2-9,
Ar₁, Ar₂, R₁, R₂, R₃, b2, and b3 are as defined in Formula 1 or 2.

20. The amine-containing compound of claim 11, wherein the amine-containing compound represented by Formula 1 or 2 is one of Compounds 1 to 385: 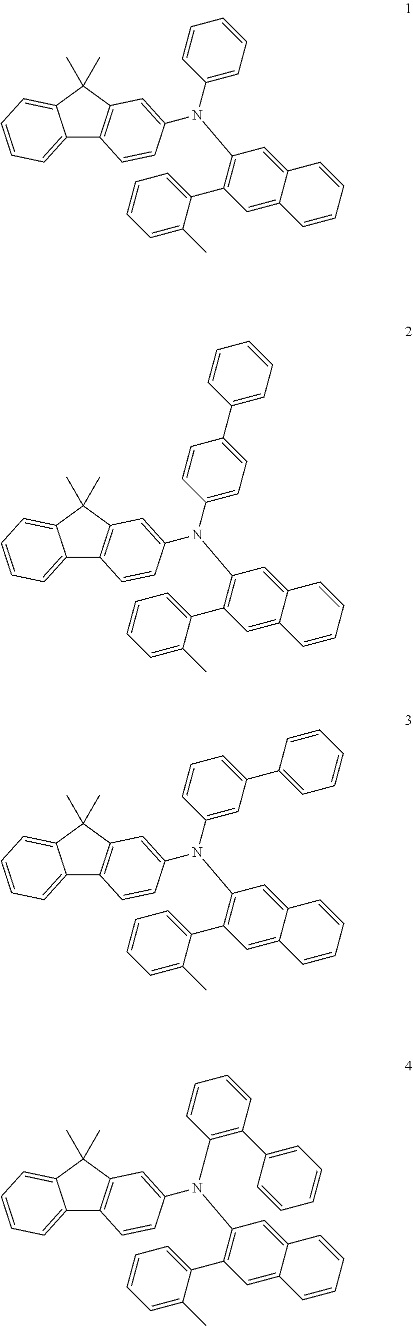 [SMILES: Cc1ccccc1-c1cc2ccccc2cc1N(c1ccccc1)c1ccc2c(c1)C(C)(C)c1ccccc1-2 ; Cc1ccccc1-c1cc2ccccc2cc1N(c1ccc(-c2ccccc2)cc1)c1ccc2c(c1)C(C)(C)c1ccccc1-2 ; Cc1ccccc1-c1cc2ccccc2cc1N(c1cccc(-c2ccccc2)c1)c1ccc2c(c1)C(C)(C)c1ccccc1-2 ; Cc1ccccc1-c1cc2ccccc2cc1N(c1ccc2c(c1)C(C)(C)c1ccccc1-2)c1ccccc1-c1ccccc1] 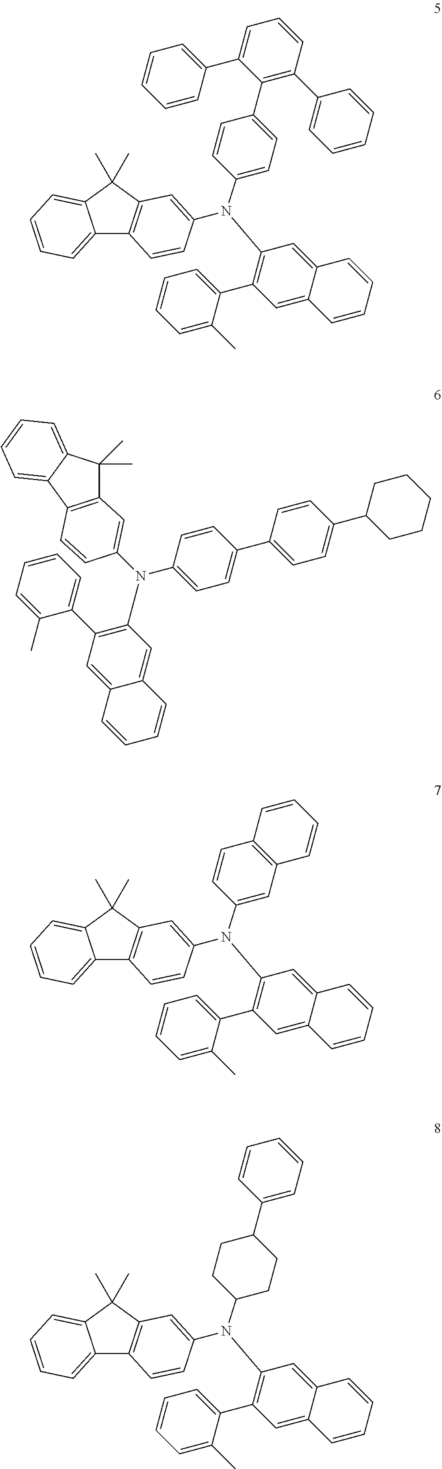 [SMILES: Cc1ccccc1-c1cc2ccccc2cc1N(c1ccc(-c2c(-c3ccccc3)cccc2-c2ccccc2)cc1)c1ccc2c(c1)C(C)(C)c1ccccc1-2 ; Cc1ccccc1-c1cc2ccccc2cc1N(c1ccc(-c2ccc(C3CCCCC3)cc2)cc1)c1ccc2c(c1)C(C)(C)c1ccccc1-2 ; Cc1ccccc1-c1cc2ccccc2cc1N(c1ccc2c(c1)C(C)(C)c1ccccc1-2)c1ccc2ccccc2c1 ; Cc1ccccc1-c1cc2ccccc2cc1N(c1ccc2c(c1)C(C)(C)c1ccccc1-2)C1CCC(c2ccccc2)CC1] 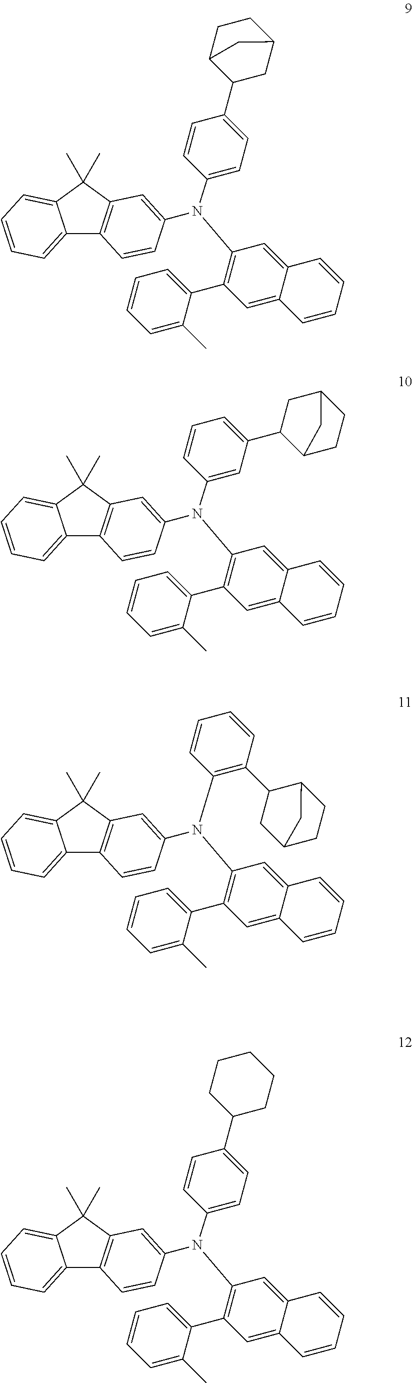 [SMILES: Cc1ccccc1-c1cc2ccccc2cc1N(c1ccc(C2CC3CCC2C3)cc1)c1ccc2c(c1)C(C)(C)c1ccccc1-2 ; Cc1ccccc1-c1cc2ccccc2cc1N(c1cccc(C2CC3CCC2C3)c1)c1ccc2c(c1)C(C)(C)c1ccccc1-2 ; Cc1ccccc1-c1cc2ccccc2cc1N(c1ccc2c(c1)C(C)(C)c1ccccc1-2)c1ccccc1C1CC2CCC1C2 ; Cc1ccccc1-c1cc2ccccc2cc1N(c1ccc(C2CCCCC2)cc1)c1ccc2c(c1)C(C)(C)c1ccccc1-2] 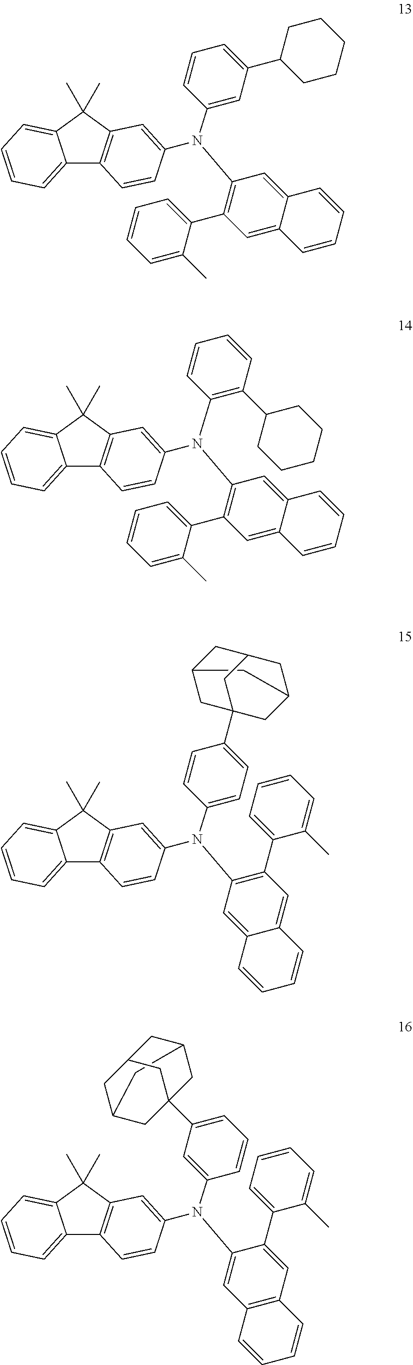 [SMILES: Cc1ccccc1-c1cc2ccccc2cc1N(c1cccc(C2CCCCC2)c1)c1ccc2c(c1)C(C)(C)c1ccccc1-2 ; Cc1ccccc1-c1cc2ccccc2cc1N(c1ccc2c(c1)C(C)(C)c1ccccc1-2)c1ccccc1C1CCCCC1 ; Cc1ccccc1-c1cc2ccccc2cc1N(c1ccc(C23CC4CC(CC(C4)C2)C3)cc1)c1ccc2c(c1)C(C)(C)c1ccccc1-2 ; Cc1ccccc1-c1cc2ccccc2cc1N(c1cccc(C23CC4CC(CC(C4)C2)C3)c1)c1ccc2c(c1)C(C)(C)c1ccccc1-2] 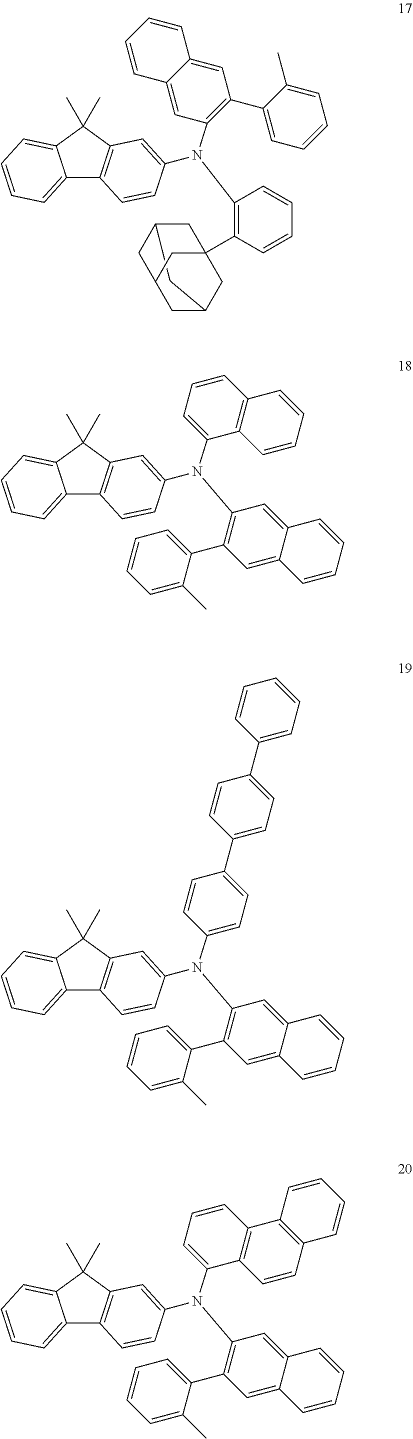 [SMILES: Cc1ccccc1-c1cc2ccccc2cc1N(c1ccc2c(c1)C(C)(C)c1ccccc1-2)c1ccccc1C12CC3CC(CC(C3)C1)C2 ; Cc1ccccc1-c1cc2ccccc2cc1N(c1ccc2c(c1)C(C)(C)c1ccccc1-2)c1cccc2ccccc12 ; Cc1ccccc1-c1cc2ccccc2cc1N(c1ccc(-c2ccc(-c3ccccc3)cc2)cc1)c1ccc2c(c1)C(C)(C)c1ccccc1-2 ; Cc1ccccc1-c1cc2ccccc2cc1N(c1ccc2c(c1)C(C)(C)c1ccccc1-2)c1cccc2c1ccc1ccccc12] 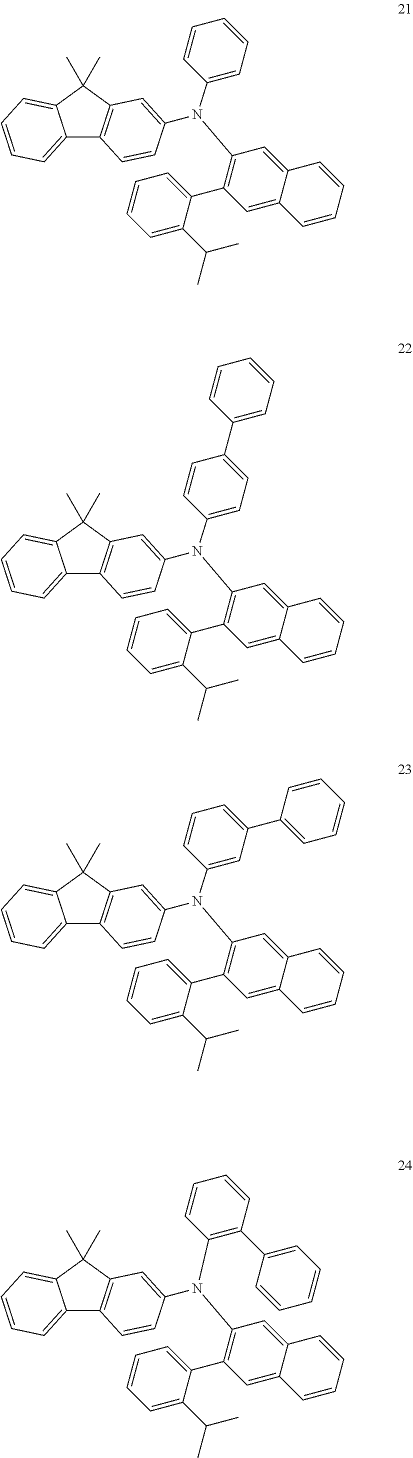 [SMILES: CC(C)c1ccccc1-c1cc2ccccc2cc1N(c1ccccc1)c1ccc2c(c1)C(C)(C)c1ccccc1-2 ; CC(C)c1ccccc1-c1cc2ccccc2cc1N(c1ccc(-c2ccccc2)cc1)c1ccc2c(c1)C(C)(C)c1ccccc1-2 ; CC(C)c1ccccc1-c1cc2ccccc2cc1N(c1cccc(-c2ccccc2)c1)c1ccc2c(c1)C(C)(C)c1ccccc1-2 ; CC(C)c1ccccc1-c1cc2ccccc2cc1N(c1ccc2c(c1)C(C)(C)c1ccccc1-2)c1ccccc1-c1ccccc1] 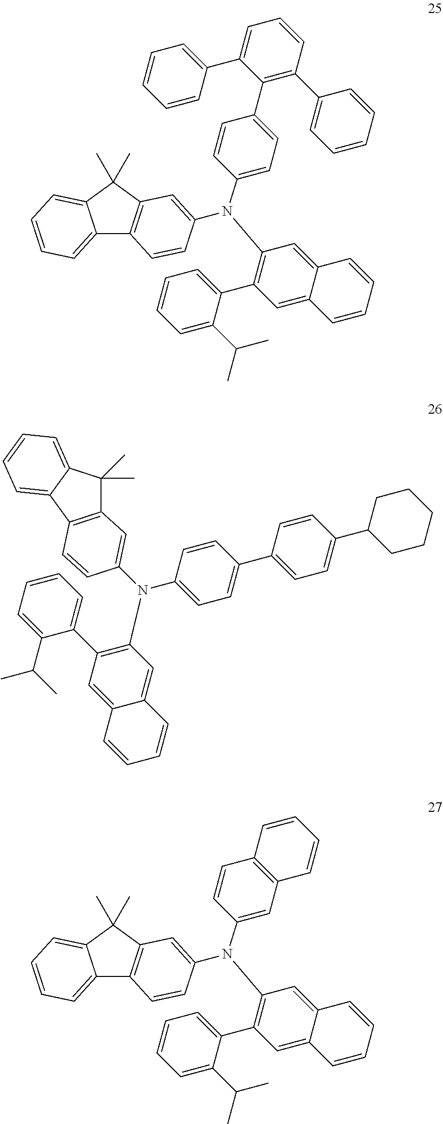 [SMILES: CC(C)c1ccccc1-c1cc2ccccc2cc1N(c1ccc(-c2c(-c3ccccc3)cccc2-c2ccccc2)cc1)c1ccc2c(c1)C(C)(C)c1ccccc1-2 ; CC(C)c1ccccc1-c1cc2ccccc2cc1N(c1ccc(-c2ccc(C3CCCCC3)cc2)cc1)c1ccc2c(c1)C(C)(C)c1ccccc1-2 ; CC(C)c1ccccc1-c1cc2ccccc2cc1N(c1ccc2c(c1)C(C)(C)c1ccccc1-2)c1ccc2ccccc2c1] 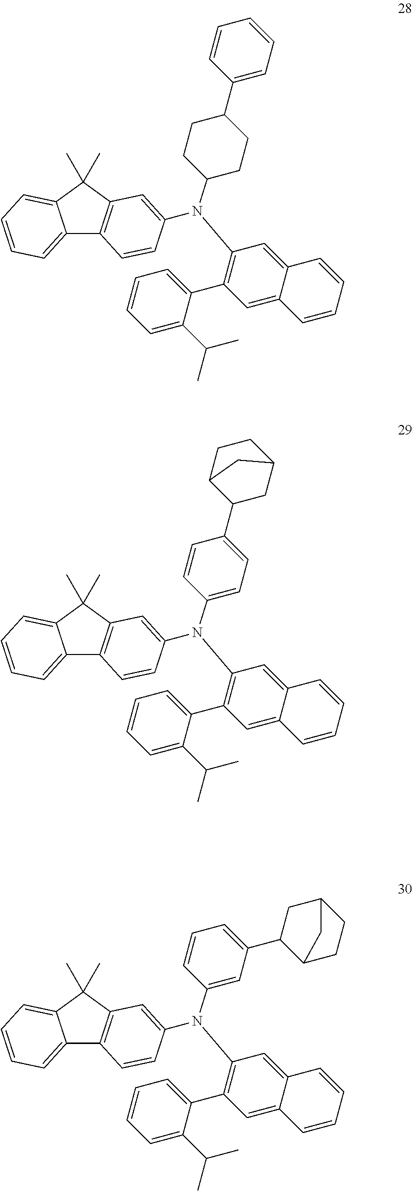 [SMILES: CC(C)c1ccccc1-c1cc2ccccc2cc1N(c1ccc2c(c1)C(C)(C)c1ccccc1-2)C1CCC(c2ccccc2)CC1 ; CC(C)c1ccccc1-c1cc2ccccc2cc1N(c1ccc(C2CC3CCC2C3)cc1)c1ccc2c(c1)C(C)(C)c1ccccc1-2 ; CC(C)c1ccccc1-c1cc2ccccc2cc1N(c1cccc(C2CC3CCC2C3)c1)c1ccc2c(c1)C(C)(C)c1ccccc1-2] 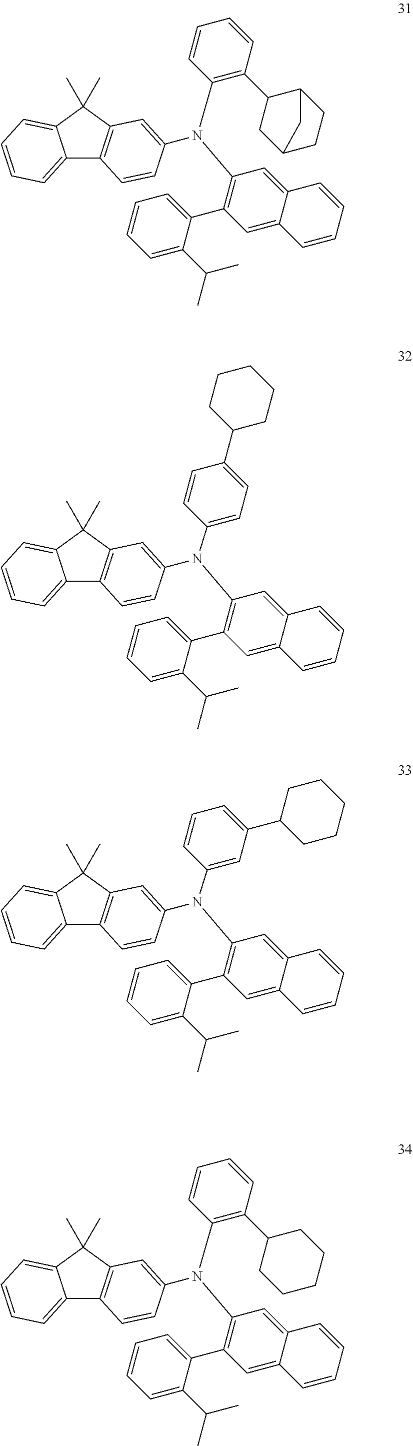 [SMILES: CC(C)c1ccccc1-c1cc2ccccc2cc1N(c1ccc2c(c1)C(C)(C)c1ccccc1-2)c1ccccc1C1CC2CCC1C2 ; CC(C)c1ccccc1-c1cc2ccccc2cc1N(c1ccc(C2CCCCC2)cc1)c1ccc2c(c1)C(C)(C)c1ccccc1-2 ; CC(C)c1ccccc1-c1cc2ccccc2cc1N(c1cccc(C2CCCCC2)c1)c1ccc2c(c1)C(C)(C)c1ccccc1-2 ; CC(C)c1ccccc1-c1cc2ccccc2cc1N(c1ccc2c(c1)C(C)(C)c1ccccc1-2)c1ccccc1C1CCCCC1] 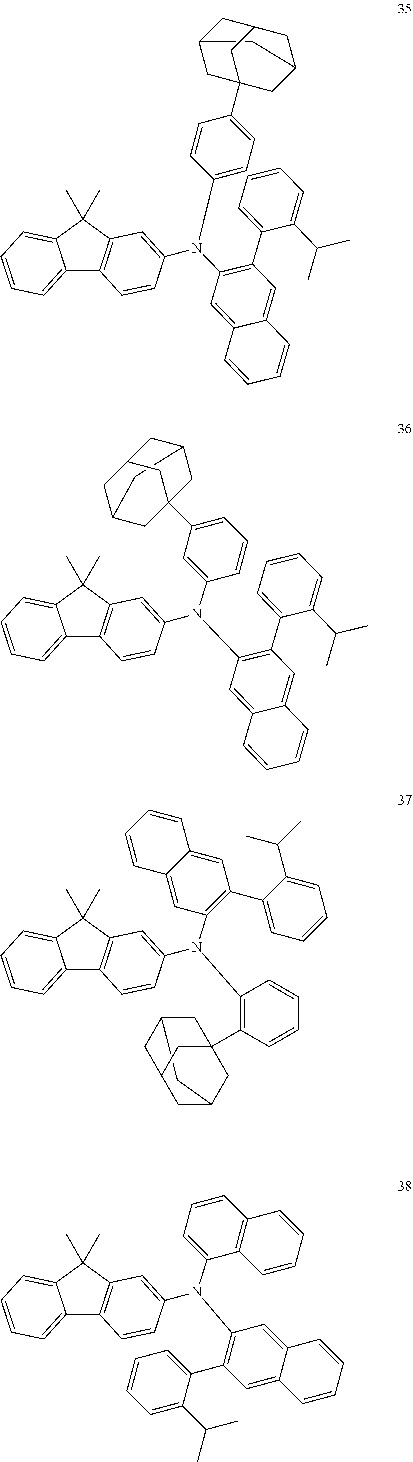 [SMILES: CC(C)c1ccccc1-c1cc2ccccc2cc1N(c1ccc(C23CC4CC(CC(C4)C2)C3)cc1)c1ccc2c(c1)C(C)(C)c1ccccc1-2 ; CC(C)c1ccccc1-c1cc2ccccc2cc1N(c1cccc(C23CC4CC(CC(C4)C2)C3)c1)c1ccc2c(c1)C(C)(C)c1ccccc1-2 ; CC(C)c1ccccc1-c1cc2ccccc2cc1N(c1ccc2c(c1)C(C)(C)c1ccccc1-2)c1ccccc1C12CC3CC(CC(C3)C1)C2 ; CC(C)c1ccccc1-c1cc2ccccc2cc1N(c1ccc2c(c1)C(C)(C)c1ccccc1-2)c1cccc2ccccc12] 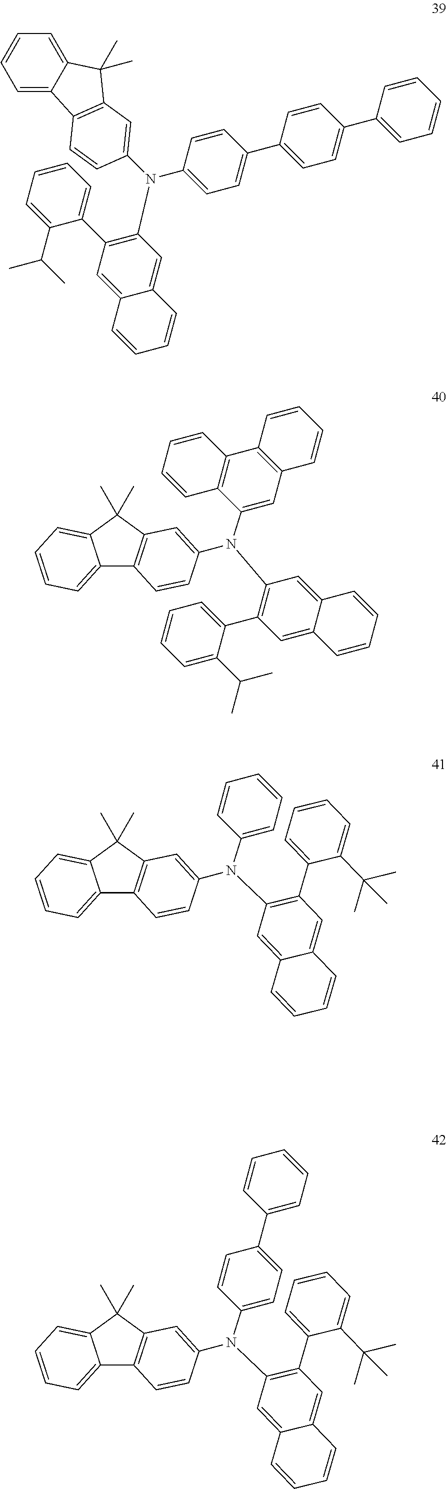 [SMILES: CC(C)c1ccccc1-c1cc2ccccc2cc1N(c1ccc(-c2ccc(-c3ccccc3)cc2)cc1)c1ccc2c(c1)C(C)(C)c1ccccc1-2 ; CC(C)c1ccccc1-c1cc2ccccc2cc1N(c1ccc2c(c1)C(C)(C)c1ccccc1-2)c1cc2ccccc2c2ccccc12 ; CC(C)(C)c1ccccc1-c1cc2ccccc2cc1N(c1ccccc1)c1ccc2c(c1)C(C)(C)c1ccccc1-2 ; CC(C)(C)c1ccccc1-c1cc2ccccc2cc1N(c1ccc(-c2ccccc2)cc1)c1ccc2c(c1)C(C)(C)c1ccccc1-2] 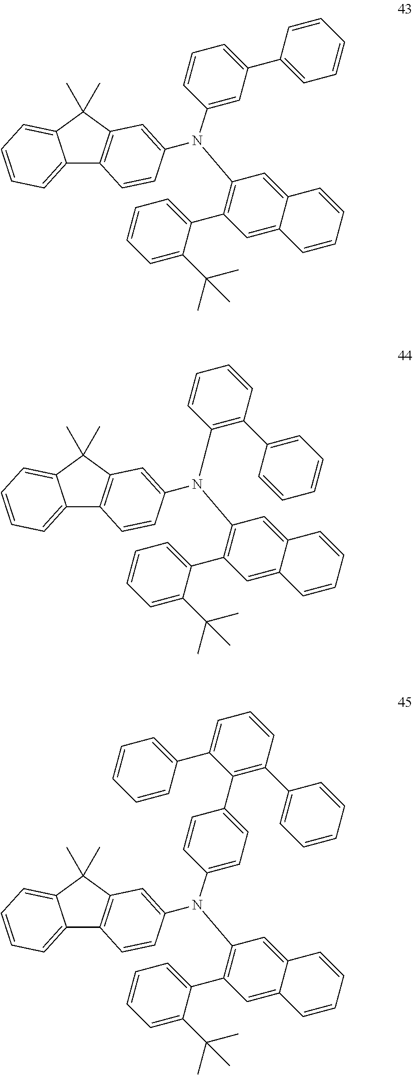 [SMILES: CC(C)(C)c1ccccc1-c1cc2ccccc2cc1N(c1cccc(-c2ccccc2)c1)c1ccc2c(c1)C(C)(C)c1ccccc1-2 ; CC(C)(C)c1ccccc1-c1cc2ccccc2cc1N(c1ccc2c(c1)C(C)(C)c1ccccc1-2)c1ccccc1-c1ccccc1 ; CC(C)(C)c1ccccc1-c1cc2ccccc2cc1N(c1ccc(-c2c(-c3ccccc3)cccc2-c2ccccc2)cc1)c1ccc2c(c1)C(C)(C)c1ccccc1-2] 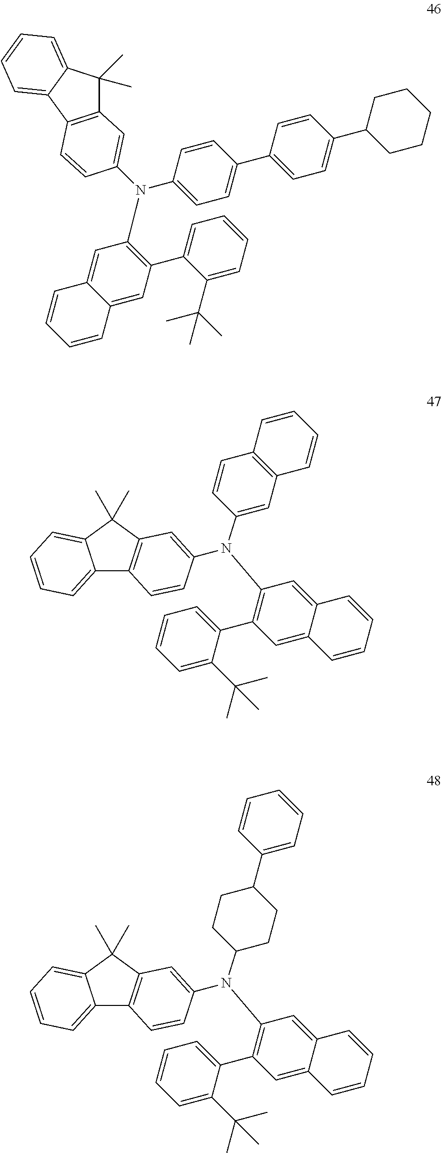 [SMILES: CC(C)(C)c1ccccc1-c1cc2ccccc2cc1N(c1ccc(-c2ccc(C3CCCCC3)cc2)cc1)c1ccc2c(c1)C(C)(C)c1ccccc1-2 ; CC(C)(C)c1ccccc1-c1cc2ccccc2cc1N(c1ccc2c(c1)C(C)(C)c1ccccc1-2)c1ccc2ccccc2c1 ; CC(C)(C)c1ccccc1-c1cc2ccccc2cc1N(c1ccc2c(c1)C(C)(C)c1ccccc1-2)C1CCC(c2ccccc2)CC1] 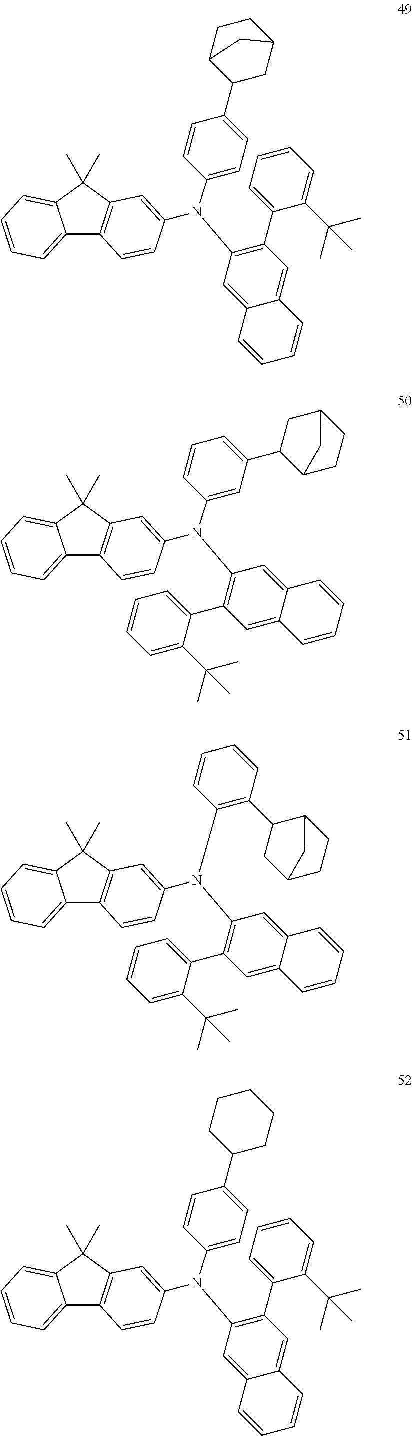 [SMILES: CC(C)(C)c1ccccc1-c1cc2ccccc2cc1N(c1ccc(C2CC3CCC2C3)cc1)c1ccc2c(c1)C(C)(C)c1ccccc1-2 ; CC(C)(C)c1ccccc1-c1cc2ccccc2cc1N(c1cccc(C2CC3CCC2C3)c1)c1ccc2c(c1)C(C)(C)c1ccccc1-2 ; CC(C)(C)c1ccccc1-c1cc2ccccc2cc1N(c1ccc2c(c1)C(C)(C)c1ccccc1-2)c1ccccc1C1CC2CCC1C2 ; CC(C)(C)c1ccccc1-c1cc2ccccc2cc1N(c1ccc(C2CCCCC2)cc1)c1ccc2c(c1)C(C)(C)c1ccccc1-2] 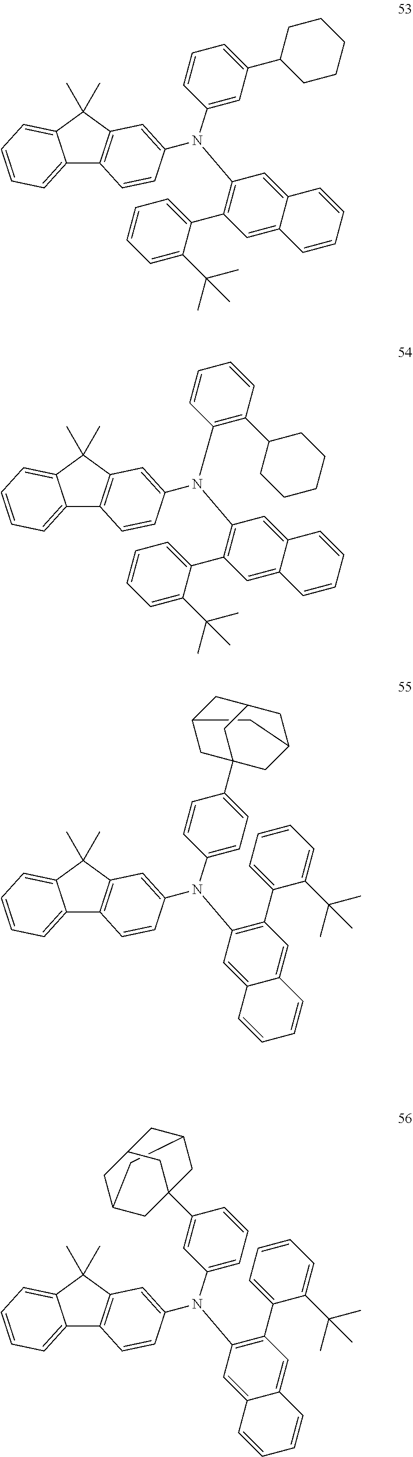 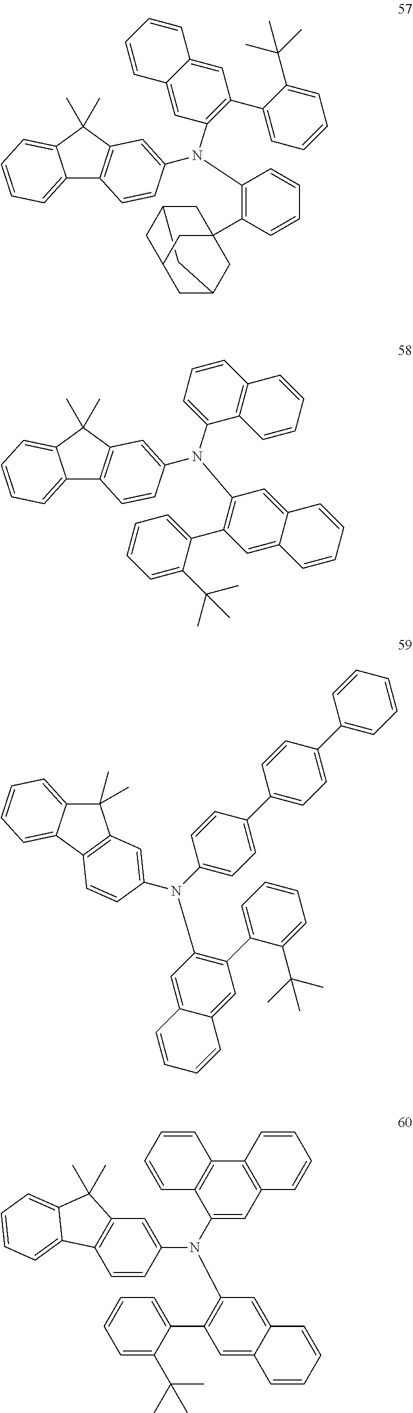 [SMILES: CC(C)(C)c1ccccc1-c1cc2ccccc2cc1N(c1ccc2c(c1)C(C)(C)c1ccccc1-2)c1ccccc1C12CC3CC(CC(C3)C1)C2 ; CC(C)(C)c1ccccc1-c1cc2ccccc2cc1N(c1ccc2c(c1)C(C)(C)c1ccccc1-2)c1cccc2ccccc12 ; CC(C)(C)c1ccccc1-c1cc2ccccc2cc1N(c1ccc(-c2ccc(-c3ccccc3)cc2)cc1)c1ccc2c(c1)C(C)(C)c1ccccc1-2 ; CC(C)(C)c1ccccc1-c1cc2ccccc2cc1N(c1ccc2c(c1)C(C)(C)c1ccccc1-2)c1cc2ccccc2c2ccccc12] 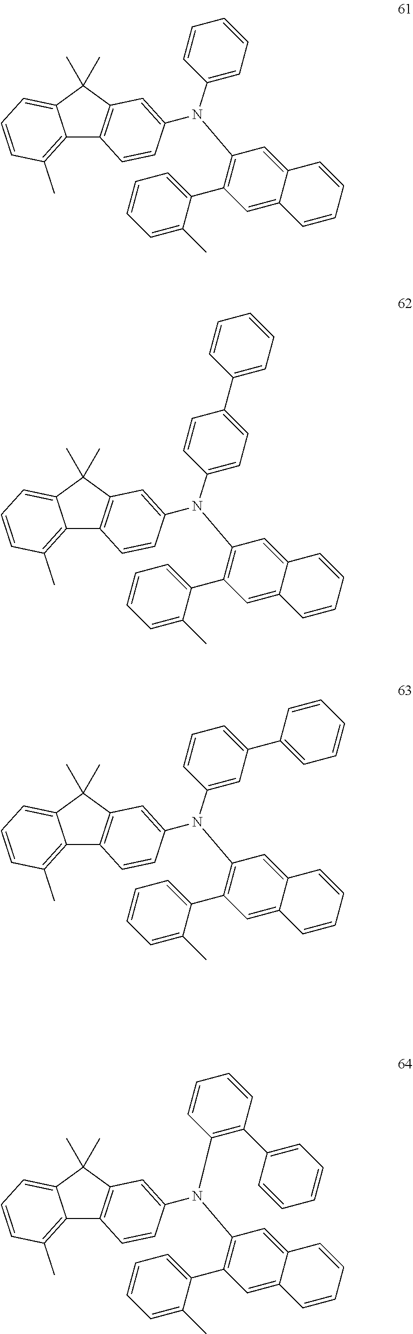 [SMILES: Cc1ccccc1-c1cc2ccccc2cc1N(c1ccccc1)c1ccc2c(c1)C(C)(C)c1cccc(C)c1-2 ; Cc1ccccc1-c1cc2ccccc2cc1N(c1ccc(-c2ccccc2)cc1)c1ccc2c(c1)C(C)(C)c1cccc(C)c1-2 ; Cc1ccccc1-c1cc2ccccc2cc1N(c1cccc(-c2ccccc2)c1)c1ccc2c(c1)C(C)(C)c1cccc(C)c1-2 ; Cc1ccccc1-c1cc2ccccc2cc1N(c1ccc2c(c1)C(C)(C)c1cccc(C)c1-2)c1ccccc1-c1ccccc1] 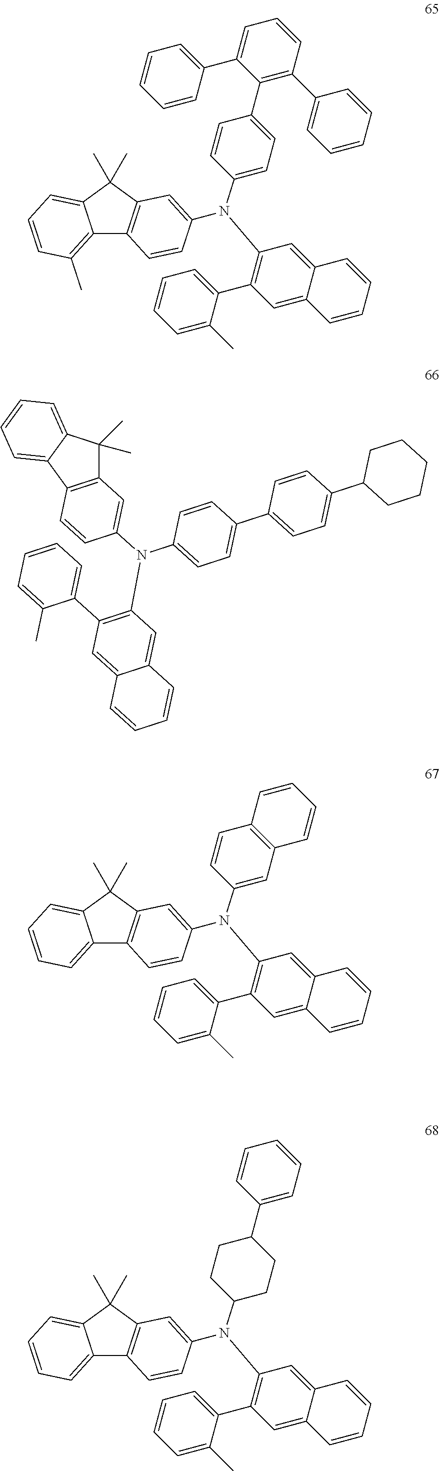 [SMILES: Cc1ccccc1-c1cc2ccccc2cc1N(c1ccc(-c2c(-c3ccccc3)cccc2-c2ccccc2)cc1)c1ccc2c(c1)C(C)(C)c1cccc(C)c1-2 ; Cc1ccccc1-c1cc2ccccc2cc1N(c1ccc(-c2ccc(C3CCCCC3)cc2)cc1)c1ccc2c(c1)C(C)(C)c1ccccc1-2 ; Cc1ccccc1-c1cc2ccccc2cc1N(c1ccc2c(c1)C(C)(C)c1ccccc1-2)c1ccc2ccccc2c1 ; Cc1ccccc1-c1cc2ccccc2cc1N(c1ccc2c(c1)C(C)(C)c1ccccc1-2)C1CCC(c2ccccc2)CC1] 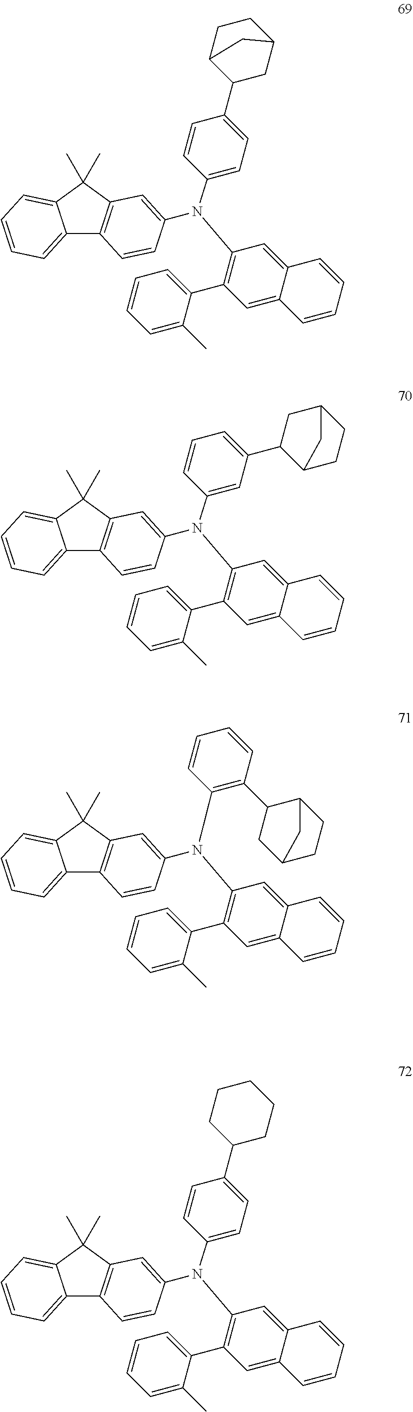 [SMILES: Cc1ccccc1-c1cc2ccccc2cc1N(c1ccc(C2CC3CCC2C3)cc1)c1ccc2c(c1)C(C)(C)c1ccccc1-2 ; Cc1ccccc1-c1cc2ccccc2cc1N(c1cccc(C2CC3CCC2C3)c1)c1ccc2c(c1)C(C)(C)c1ccccc1-2 ; Cc1ccccc1-c1cc2ccccc2cc1N(c1ccc2c(c1)C(C)(C)c1ccccc1-2)c1ccccc1C1CC2CCC1C2 ; Cc1ccccc1-c1cc2ccccc2cc1N(c1ccc(C2CCCCC2)cc1)c1ccc2c(c1)C(C)(C)c1ccccc1-2] 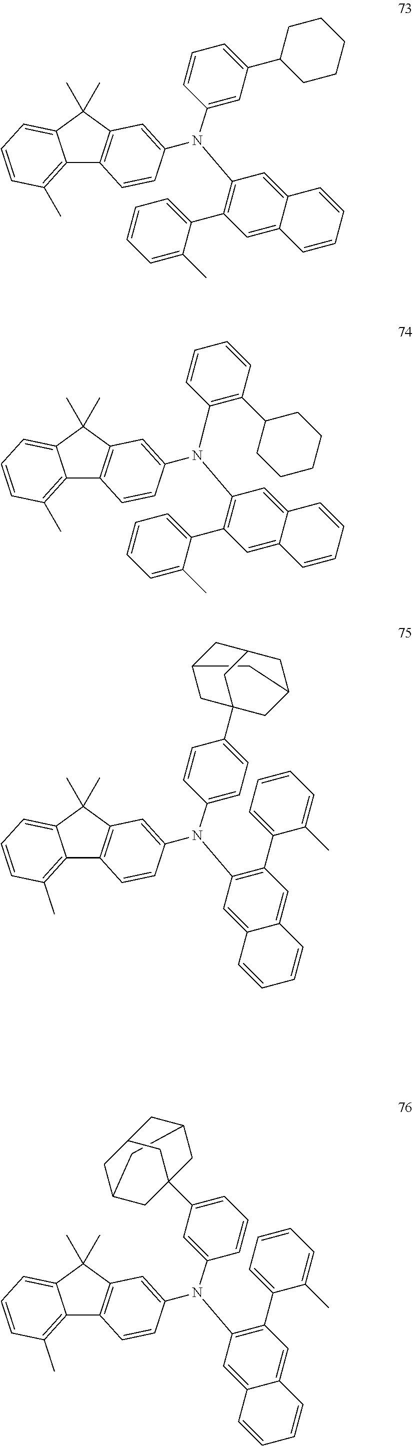 [SMILES: Cc1ccccc1-c1cc2ccccc2cc1N(c1cccc(C2CCCCC2)c1)c1ccc2c(c1)C(C)(C)c1cccc(C)c1-2 ; Cc1ccccc1-c1cc2ccccc2cc1N(c1ccc2c(c1)C(C)(C)c1cccc(C)c1-2)c1ccccc1C1CCCCC1 ; Cc1ccccc1-c1cc2ccccc2cc1N(c1ccc(C23CC4CC(CC(C4)C2)C3)cc1)c1ccc2c(c1)C(C)(C)c1cccc(C)c1-2 ; Cc1ccccc1-c1cc2ccccc2cc1N(c1cccc(C23CC4CC(CC(C4)C2)C3)c1)c1ccc2c(c1)C(C)(C)c1cccc(C)c1-2] 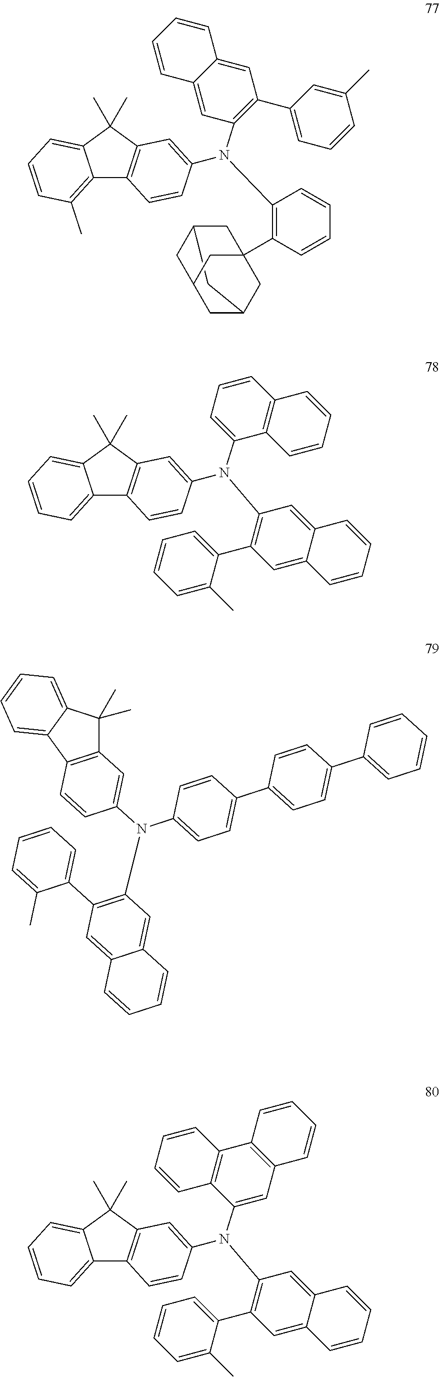 [SMILES: Cc1cccc(-c2cc3ccccc3cc2N(c2ccc3c(c2)C(C)(C)c2cccc(C)c2-3)c2ccccc2C23CC4CC(CC(C4)C2)C3)c1 ; Cc1ccccc1-c1cc2ccccc2cc1N(c1ccc2c(c1)C(C)(C)c1ccccc1-2)c1cccc2ccccc12 ; Cc1ccccc1-c1cc2ccccc2cc1N(c1ccc(-c2ccc(-c3ccccc3)cc2)cc1)c1ccc2c(c1)C(C)(C)c1ccccc1-2 ; Cc1ccccc1-c1cc2ccccc2cc1N(c1ccc2c(c1)C(C)(C)c1ccccc1-2)c1cc2ccccc2c2ccccc12] 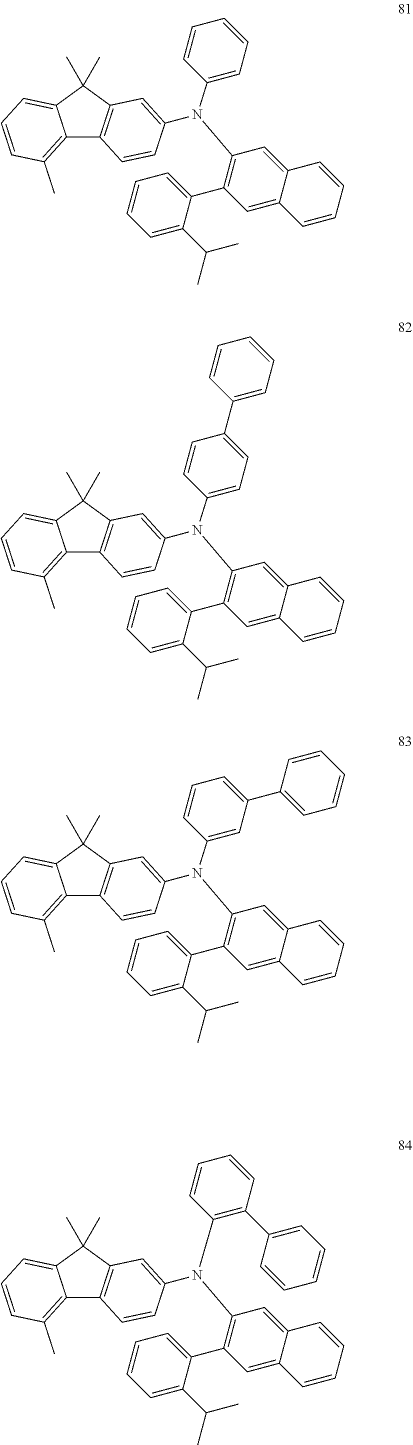 [SMILES: Cc1cccc2c1-c1ccc(N(c3ccccc3)c3cc4ccccc4cc3-c3ccccc3C(C)C)cc1C2(C)C ; Cc1cccc2c1-c1ccc(N(c3ccc(-c4ccccc4)cc3)c3cc4ccccc4cc3-c3ccccc3C(C)C)cc1C2(C)C ; Cc1cccc2c1-c1ccc(N(c3cccc(-c4ccccc4)c3)c3cc4ccccc4cc3-c3ccccc3C(C)C)cc1C2(C)C ; Cc1cccc2c1-c1ccc(N(c3ccccc3-c3ccccc3)c3cc4ccccc4cc3-c3ccccc3C(C)C)cc1C2(C)C] 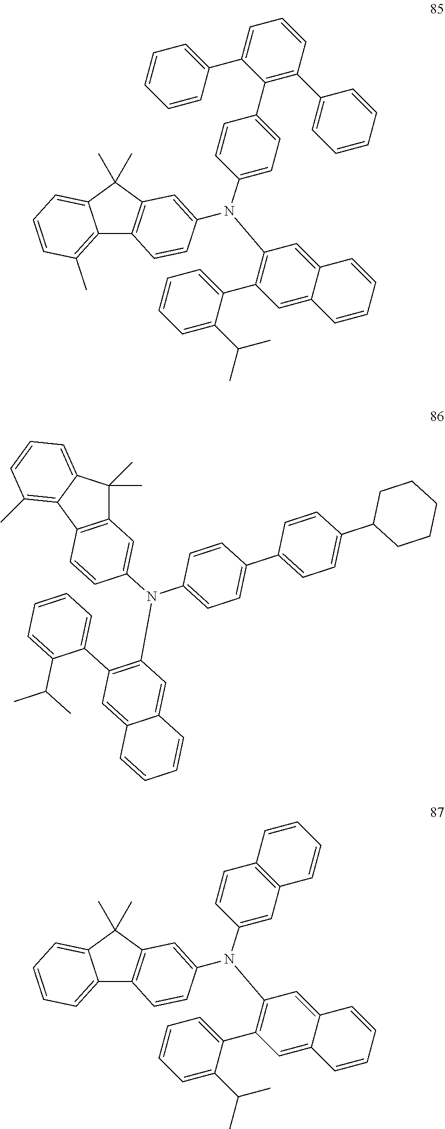 [SMILES: Cc1cccc2c1-c1ccc(N(c3ccc(-c4c(-c5ccccc5)cccc4-c4ccccc4)cc3)c3cc4ccccc4cc3-c3ccccc3C(C)C)cc1C2(C)C ; Cc1cccc2c1-c1ccc(N(c3ccc(-c4ccc(C5CCCCC5)cc4)cc3)c3cc4ccccc4cc3-c3ccccc3C(C)C)cc1C2(C)C ; CC(C)c1ccccc1-c1cc2ccccc2cc1N(c1ccc2c(c1)C(C)(C)c1ccccc1-2)c1ccc2ccccc2c1] 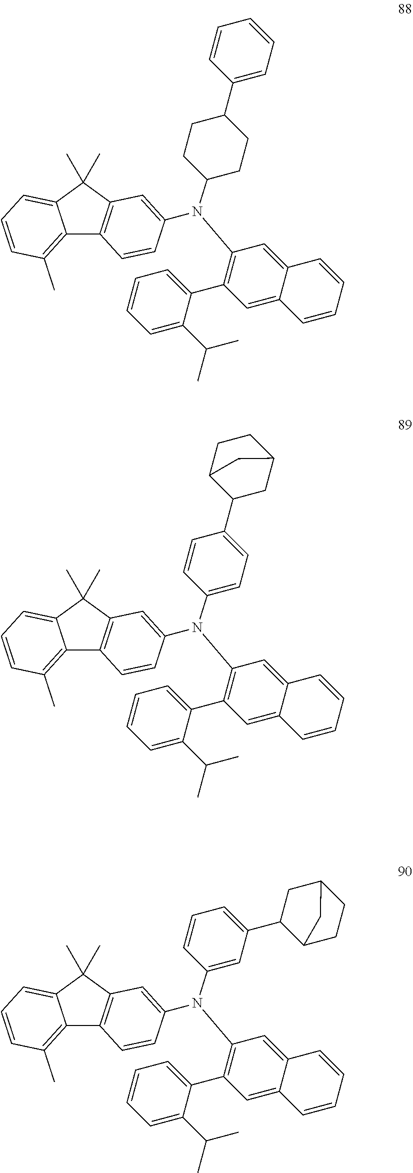 [SMILES: Cc1cccc2c1-c1ccc(N(c3cc4ccccc4cc3-c3ccccc3C(C)C)C3CCC(c4ccccc4)CC3)cc1C2(C)C ; Cc1cccc2c1-c1ccc(N(c3ccc(C4CC5CCC4C5)cc3)c3cc4ccccc4cc3-c3ccccc3C(C)C)cc1C2(C)C ; Cc1cccc2c1-c1ccc(N(c3cccc(C4CC5CCC4C5)c3)c3cc4ccccc4cc3-c3ccccc3C(C)C)cc1C2(C)C] 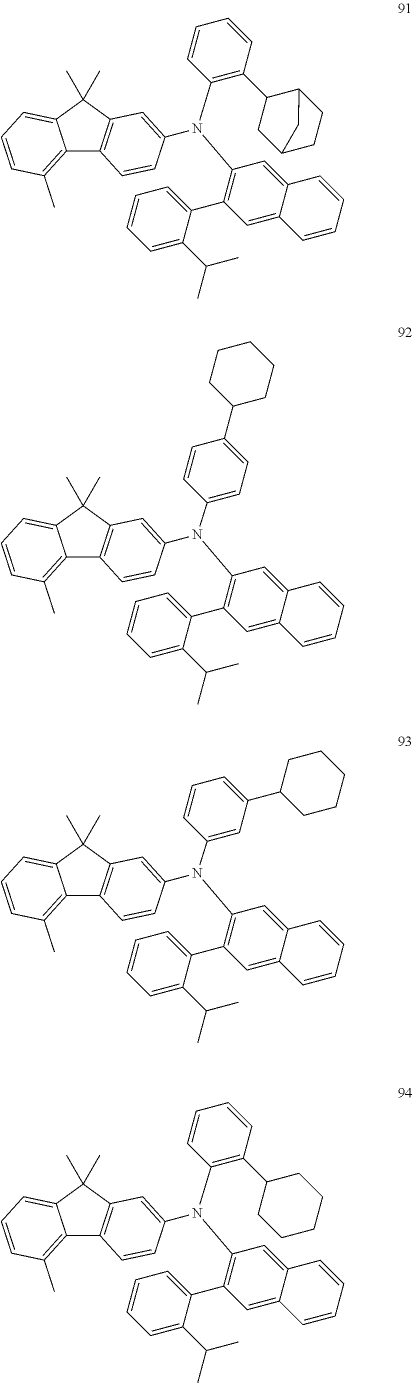 [SMILES: Cc1cccc2c1-c1ccc(N(c3cc4ccccc4cc3-c3ccccc3C(C)C)c3ccccc3C3CC4CCC3C4)cc1C2(C)C ; Cc1cccc2c1-c1ccc(N(c3ccc(C4CCCCC4)cc3)c3cc4ccccc4cc3-c3ccccc3C(C)C)cc1C2(C)C ; Cc1cccc2c1-c1ccc(N(c3cccc(C4CCCCC4)c3)c3cc4ccccc4cc3-c3ccccc3C(C)C)cc1C2(C)C ; Cc1cccc2c1-c1ccc(N(c3cc4ccccc4cc3-c3ccccc3C(C)C)c3ccccc3C3CCCCC3)cc1C2(C)C] 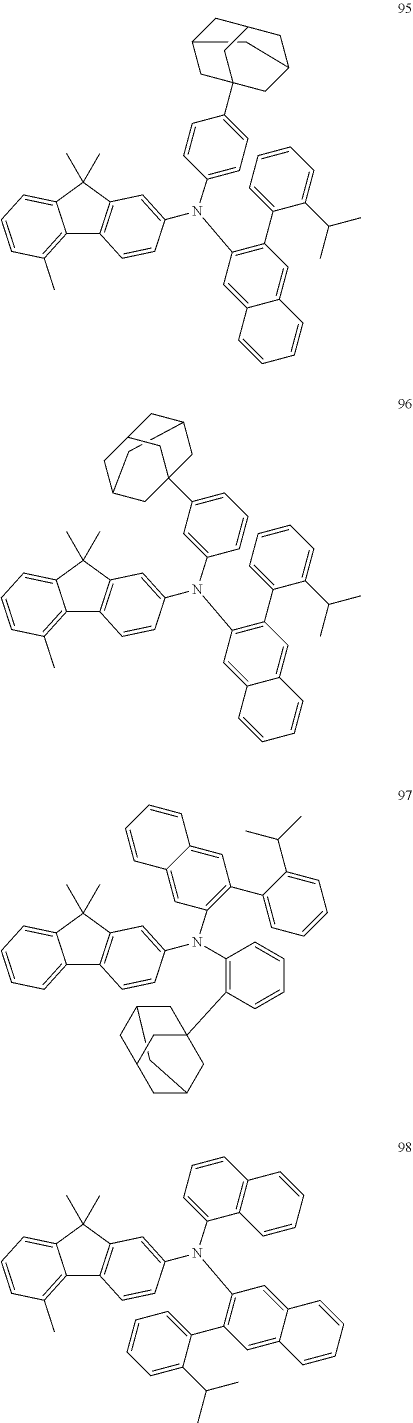 [SMILES: Cc1cccc2c1-c1ccc(N(c3ccc(C45CC6CC(CC(C6)C4)C5)cc3)c3cc4ccccc4cc3-c3ccccc3C(C)C)cc1C2(C)C ; Cc1cccc2c1-c1ccc(N(c3cccc(C45CC6CC(CC(C6)C4)C5)c3)c3cc4ccccc4cc3-c3ccccc3C(C)C)cc1C2(C)C ; CC(C)c1ccccc1-c1cc2ccccc2cc1N(c1ccc2c(c1)C(C)(C)c1ccccc1-2)c1ccccc1C12CC3CC(CC(C3)C1)C2 ; Cc1cccc2c1-c1ccc(N(c3cc4ccccc4cc3-c3ccccc3C(C)C)c3cccc4ccccc34)cc1C2(C)C] 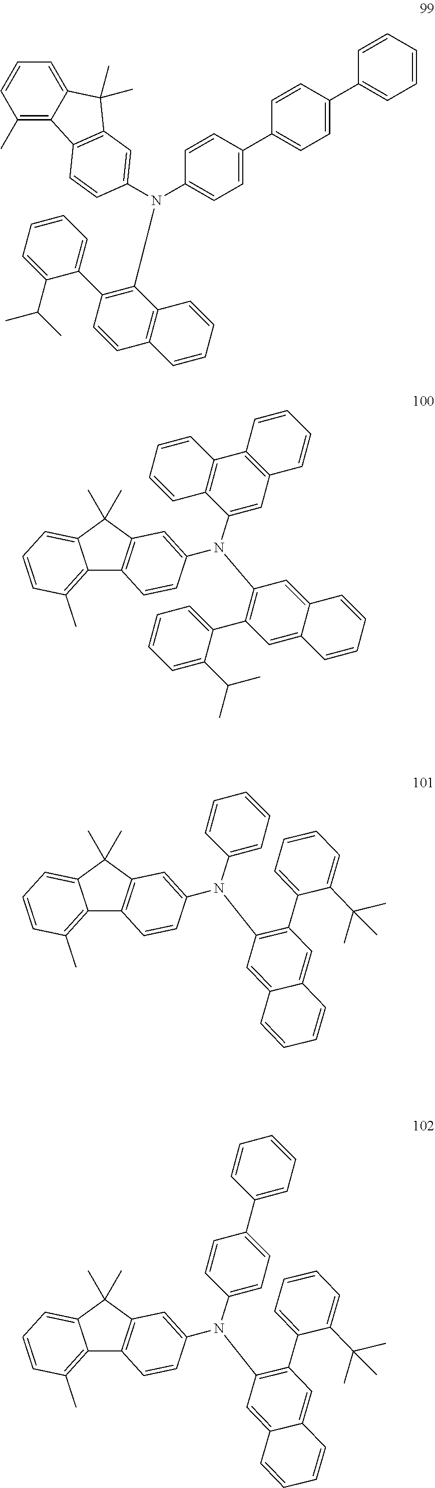 [SMILES: Cc1cccc2c1-c1ccc(N(c3ccc(-c4ccc(-c5ccccc5)cc4)cc3)c3c(-c4ccccc4C(C)C)ccc4ccccc34)cc1C2(C)C ; Cc1cccc2c1-c1ccc(N(c3cc4ccccc4cc3-c3ccccc3C(C)C)c3cc4ccccc4c4ccccc34)cc1C2(C)C ; Cc1cccc2c1-c1ccc(N(c3ccccc3)c3cc4ccccc4cc3-c3ccccc3C(C)(C)C)cc1C2(C)C ; Cc1cccc2c1-c1ccc(N(c3ccc(-c4ccccc4)cc3)c3cc4ccccc4cc3-c3ccccc3C(C)(C)C)cc1C2(C)C] 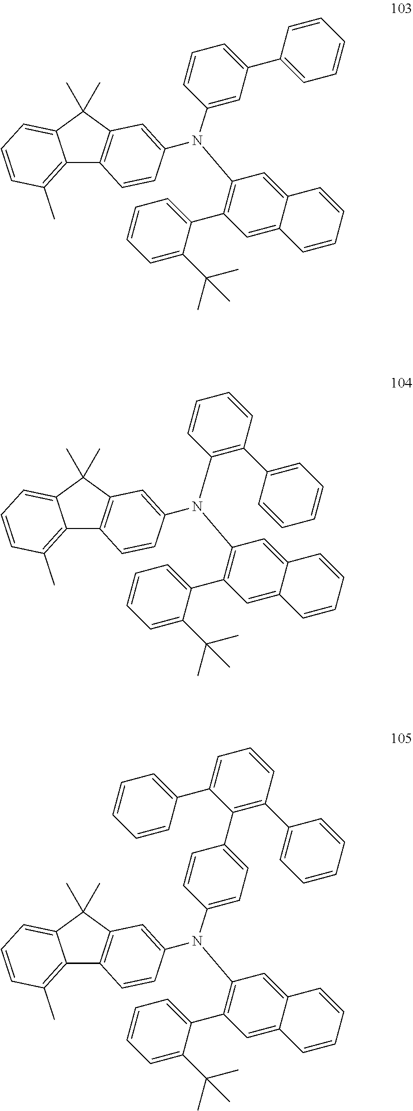 [SMILES: Cc1cccc2c1-c1ccc(N(c3cccc(-c4ccccc4)c3)c3cc4ccccc4cc3-c3ccccc3C(C)(C)C)cc1C2(C)C ; Cc1cccc2c1-c1ccc(N(c3ccccc3-c3ccccc3)c3cc4ccccc4cc3-c3ccccc3C(C)(C)C)cc1C2(C)C ; Cc1cccc2c1-c1ccc(N(c3ccc(-c4c(-c5ccccc5)cccc4-c4ccccc4)cc3)c3cc4ccccc4cc3-c3ccccc3C(C)(C)C)cc1C2(C)C] 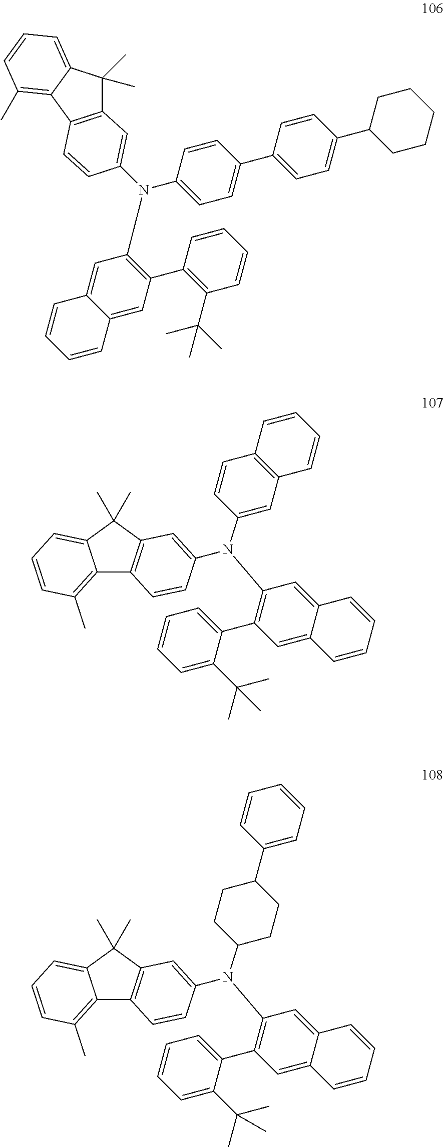 [SMILES: Cc1cccc2c1-c1ccc(N(c3ccc(-c4ccc(C5CCCCC5)cc4)cc3)c3cc4ccccc4cc3-c3ccccc3C(C)(C)C)cc1C2(C)C ; Cc1cccc2c1-c1ccc(N(c3ccc4ccccc4c3)c3cc4ccccc4cc3-c3ccccc3C(C)(C)C)cc1C2(C)C ; Cc1cccc2c1-c1ccc(N(c3cc4ccccc4cc3-c3ccccc3C(C)(C)C)C3CCC(c4ccccc4)CC3)cc1C2(C)C] 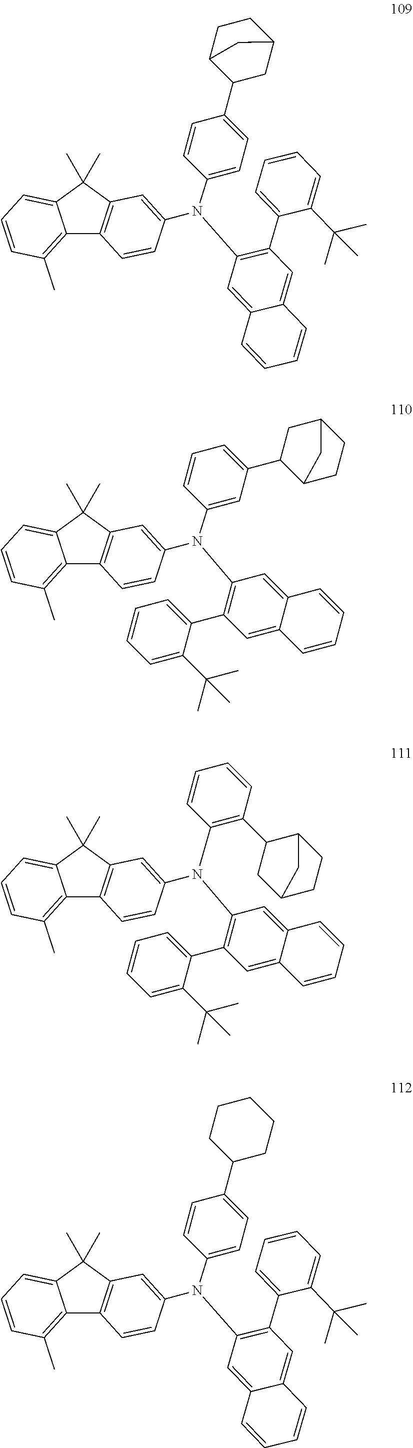 [SMILES: Cc1cccc2c1-c1ccc(N(c3ccc(C4CC5CCC4C5)cc3)c3cc4ccccc4cc3-c3ccccc3C(C)(C)C)cc1C2(C)C ; Cc1cccc2c1-c1ccc(N(c3cccc(C4CC5CCC4C5)c3)c3cc4ccccc4cc3-c3ccccc3C(C)(C)C)cc1C2(C)C ; Cc1cccc2c1-c1ccc(N(c3cc4ccccc4cc3-c3ccccc3C(C)(C)C)c3ccccc3C3CC4CCC3C4)cc1C2(C)C ; Cc1cccc2c1-c1ccc(N(c3ccc(C4CCCCC4)cc3)c3cc4ccccc4cc3-c3ccccc3C(C)(C)C)cc1C2(C)C] 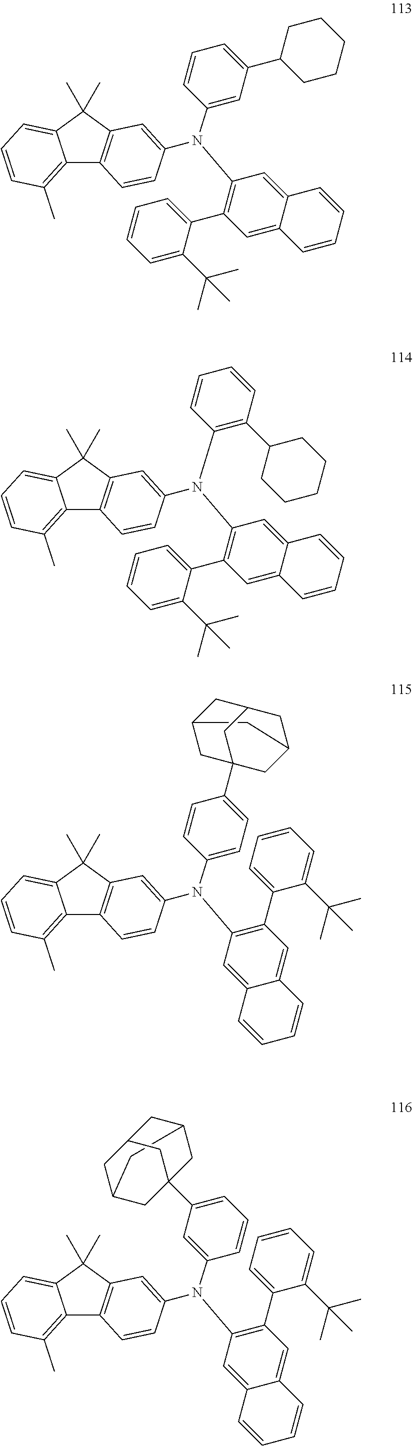 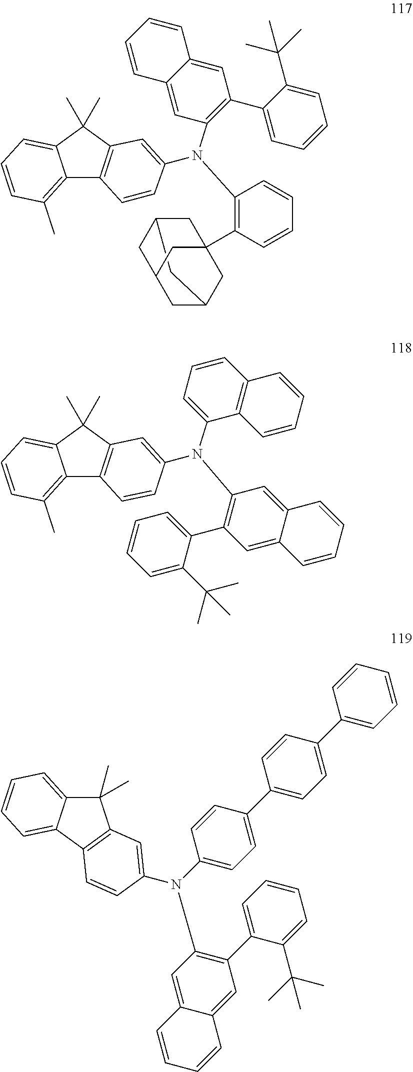 [SMILES: Cc1cccc2c1-c1ccc(N(c3cc4ccccc4cc3-c3ccccc3C(C)(C)C)c3ccccc3C34CC5CC(CC(C5)C3)C4)cc1C2(C)C ; Cc1cccc2c1-c1ccc(N(c3cc4ccccc4cc3-c3ccccc3C(C)(C)C)c3cccc4ccccc34)cc1C2(C)C ; CC(C)(C)c1ccccc1-c1cc2ccccc2cc1N(c1ccc(-c2ccc(-c3ccccc3)cc2)cc1)c1ccc2c(c1)C(C)(C)c1ccccc1-2] 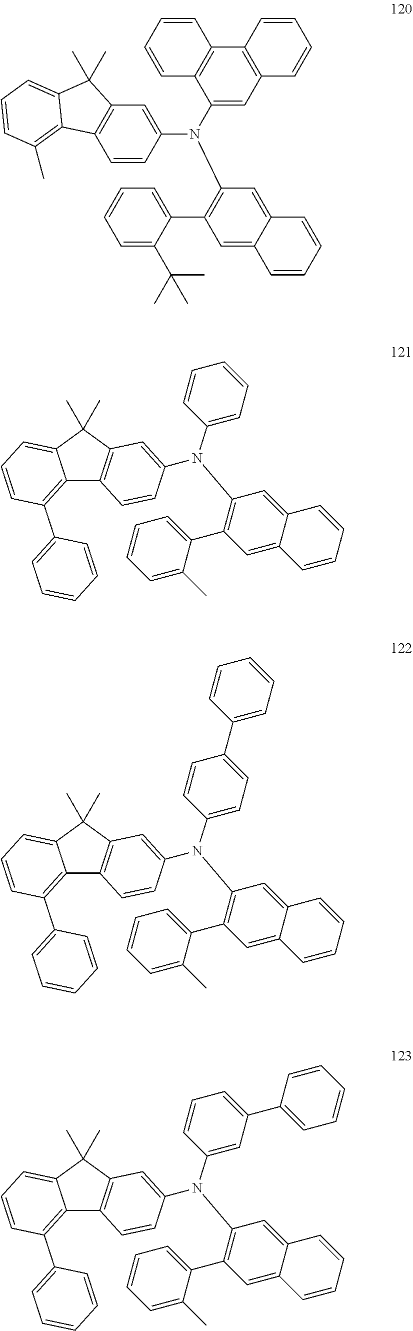 [SMILES: Cc1cccc2c1-c1ccc(N(c3cc4ccccc4cc3-c3ccccc3C(C)(C)C)c3cc4ccccc4c4ccccc34)cc1C2(C)C ; Cc1ccccc1-c1cc2ccccc2cc1N(c1ccccc1)c1ccc2c(c1)C(C)(C)c1cccc(-c3ccccc3)c1-2 ; Cc1ccccc1-c1cc2ccccc2cc1N(c1ccc(-c2ccccc2)cc1)c1ccc2c(c1)C(C)(C)c1cccc(-c3ccccc3)c1-2 ; Cc1ccccc1-c1cc2ccccc2cc1N(c1cccc(-c2ccccc2)c1)c1ccc2c(c1)C(C)(C)c1cccc(-c3ccccc3)c1-2] 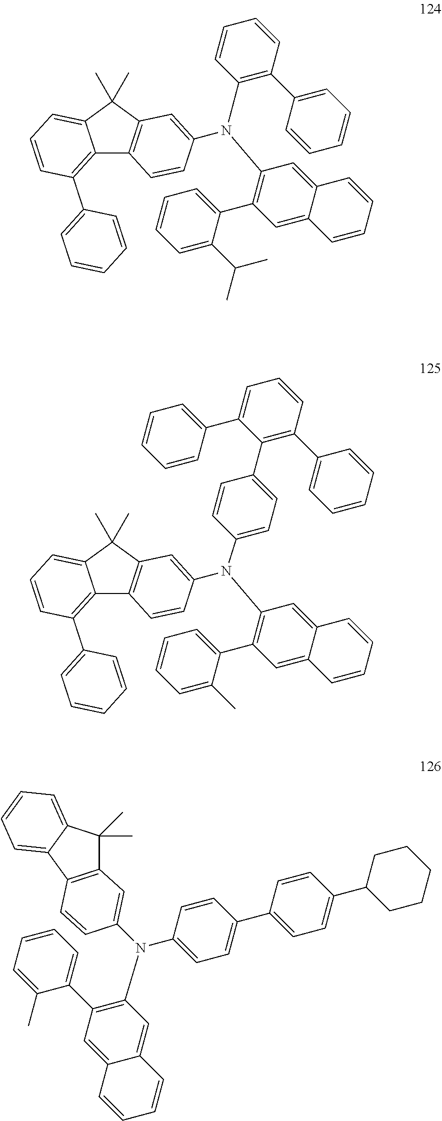 [SMILES: CC(C)c1ccccc1-c1cc2ccccc2cc1N(c1ccc2c(c1)C(C)(C)c1cccc(-c3ccccc3)c1-2)c1ccccc1-c1ccccc1 ; Cc1ccccc1-c1cc2ccccc2cc1N(c1ccc(-c2c(-c3ccccc3)cccc2-c2ccccc2)cc1)c1ccc2c(c1)C(C)(C)c1cccc(-c3ccccc3)c1-2 ; Cc1ccccc1-c1cc2ccccc2cc1N(c1ccc(-c2ccc(C3CCCCC3)cc2)cc1)c1ccc2c(c1)C(C)(C)c1ccccc1-2] 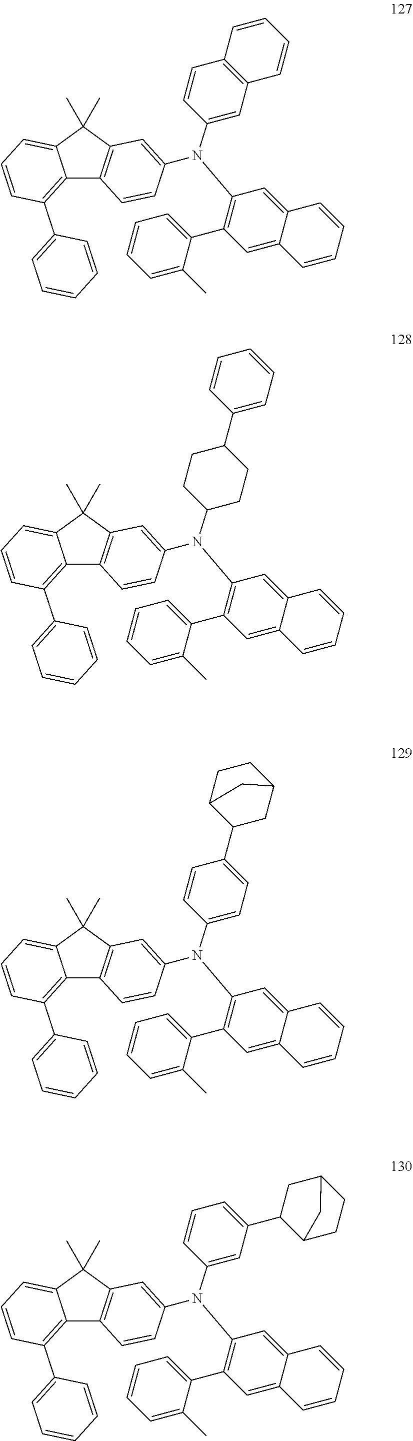 [SMILES: Cc1ccccc1-c1cc2ccccc2cc1N(c1ccc2c(c1)C(C)(C)c1cccc(-c3ccccc3)c1-2)c1ccc2ccccc2c1 ; Cc1ccccc1-c1cc2ccccc2cc1N(c1ccc2c(c1)C(C)(C)c1cccc(-c3ccccc3)c1-2)C1CCC(c2ccccc2)CC1 ; Cc1ccccc1-c1cc2ccccc2cc1N(c1ccc(C2CC3CCC2C3)cc1)c1ccc2c(c1)C(C)(C)c1cccc(-c3ccccc3)c1-2 ; Cc1ccccc1-c1cc2ccccc2cc1N(c1cccc(C2CC3CCC2C3)c1)c1ccc2c(c1)C(C)(C)c1cccc(-c3ccccc3)c1-2] 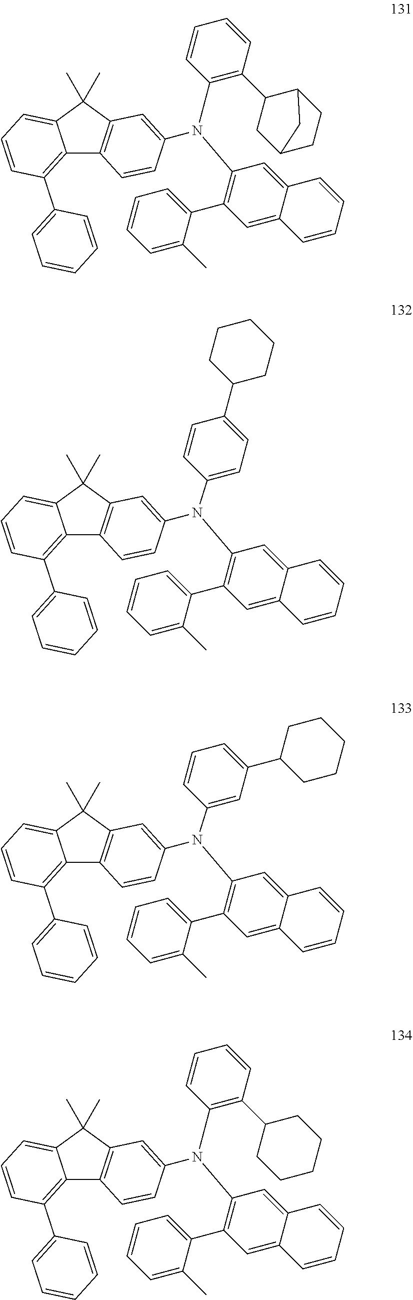 [SMILES: Cc1ccccc1-c1cc2ccccc2cc1N(c1ccc2c(c1)C(C)(C)c1cccc(-c3ccccc3)c1-2)c1ccccc1C1CC2CCC1C2 ; Cc1ccccc1-c1cc2ccccc2cc1N(c1ccc(C2CCCCC2)cc1)c1ccc2c(c1)C(C)(C)c1cccc(-c3ccccc3)c1-2 ; Cc1ccccc1-c1cc2ccccc2cc1N(c1cccc(C2CCCCC2)c1)c1ccc2c(c1)C(C)(C)c1cccc(-c3ccccc3)c1-2 ; Cc1ccccc1-c1cc2ccccc2cc1N(c1ccc2c(c1)C(C)(C)c1cccc(-c3ccccc3)c1-2)c1ccccc1C1CCCCC1] 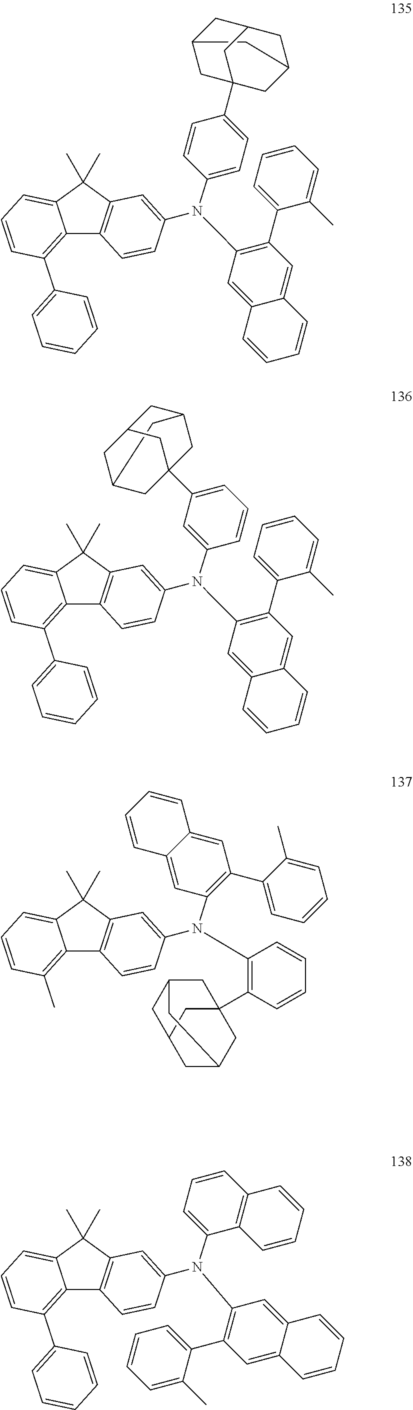 [SMILES: Cc1ccccc1-c1cc2ccccc2cc1N(c1ccc(C23CC4CC(CC(C4)C2)C3)cc1)c1ccc2c(c1)C(C)(C)c1cccc(-c3ccccc3)c1-2 ; Cc1ccccc1-c1cc2ccccc2cc1N(c1cccc(C23CC4CC(CC(C4)C2)C3)c1)c1ccc2c(c1)C(C)(C)c1cccc(-c3ccccc3)c1-2 ; Cc1ccccc1-c1cc2ccccc2cc1N(c1ccc2c(c1)C(C)(C)c1cccc(C)c1-2)c1ccccc1C12CC3CC(CC(C3)C1)C2 ; Cc1ccccc1-c1cc2ccccc2cc1N(c1ccc2c(c1)C(C)(C)c1cccc(-c3ccccc3)c1-2)c1cccc2ccccc12] 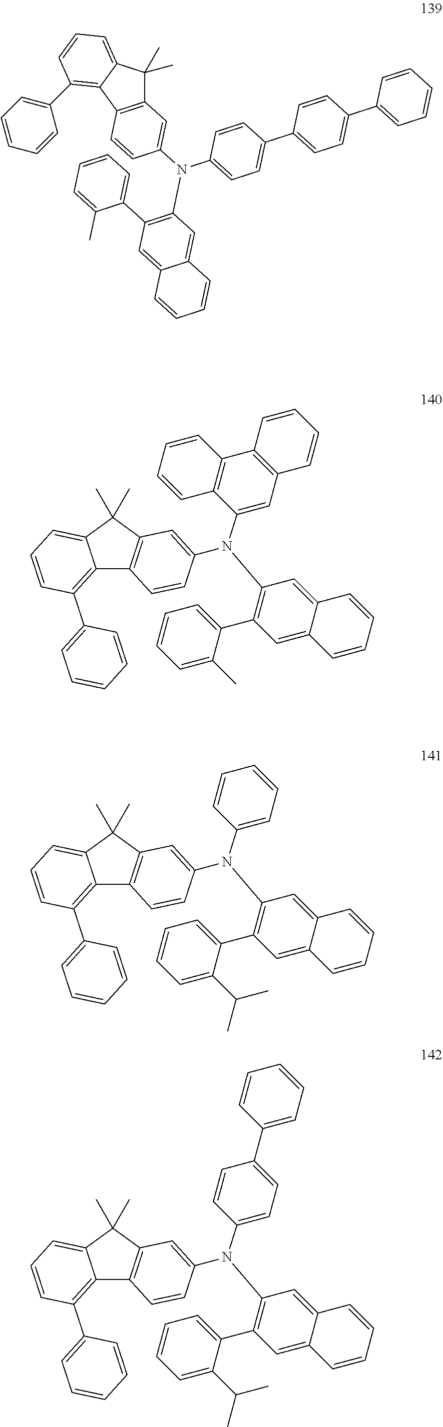 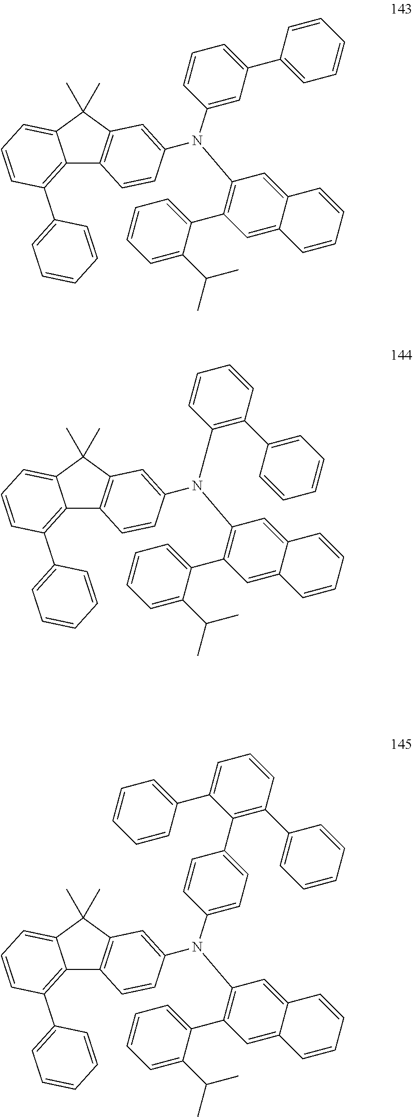 [SMILES: CC(C)c1ccccc1-c1cc2ccccc2cc1N(c1cccc(-c2ccccc2)c1)c1ccc2c(c1)C(C)(C)c1cccc(-c3ccccc3)c1-2 ; CC(C)c1ccccc1-c1cc2ccccc2cc1N(c1ccc2c(c1)C(C)(C)c1cccc(-c3ccccc3)c1-2)c1ccccc1-c1ccccc1 ; CC(C)c1ccccc1-c1cc2ccccc2cc1N(c1ccc(-c2c(-c3ccccc3)cccc2-c2ccccc2)cc1)c1ccc2c(c1)C(C)(C)c1cccc(-c3ccccc3)c1-2] 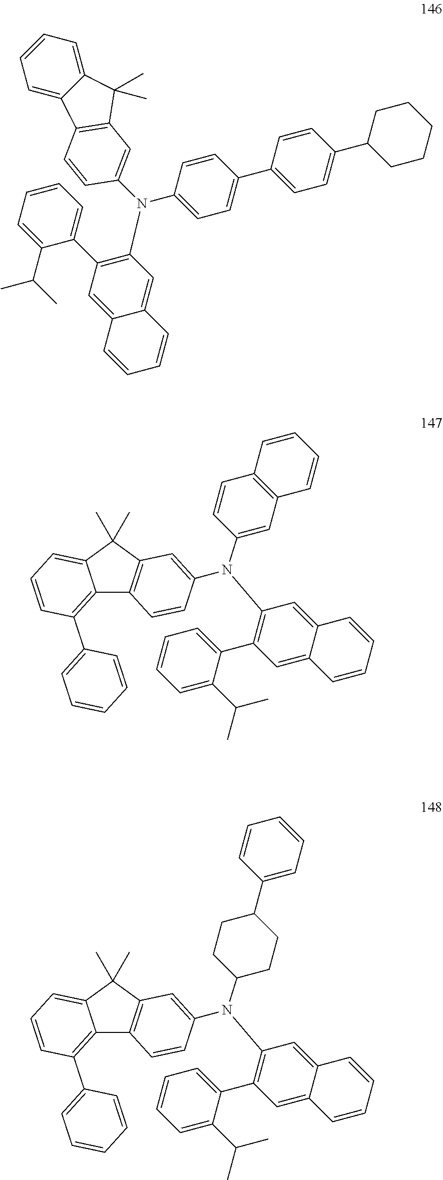 [SMILES: CC(C)c1ccccc1-c1cc2ccccc2cc1N(c1ccc(-c2ccc(C3CCCCC3)cc2)cc1)c1ccc2c(c1)C(C)(C)c1ccccc1-2 ; CC(C)c1ccccc1-c1cc2ccccc2cc1N(c1ccc2c(c1)C(C)(C)c1cccc(-c3ccccc3)c1-2)c1ccc2ccccc2c1 ; CC(C)c1ccccc1-c1cc2ccccc2cc1N(c1ccc2c(c1)C(C)(C)c1cccc(-c3ccccc3)c1-2)C1CCC(c2ccccc2)CC1] 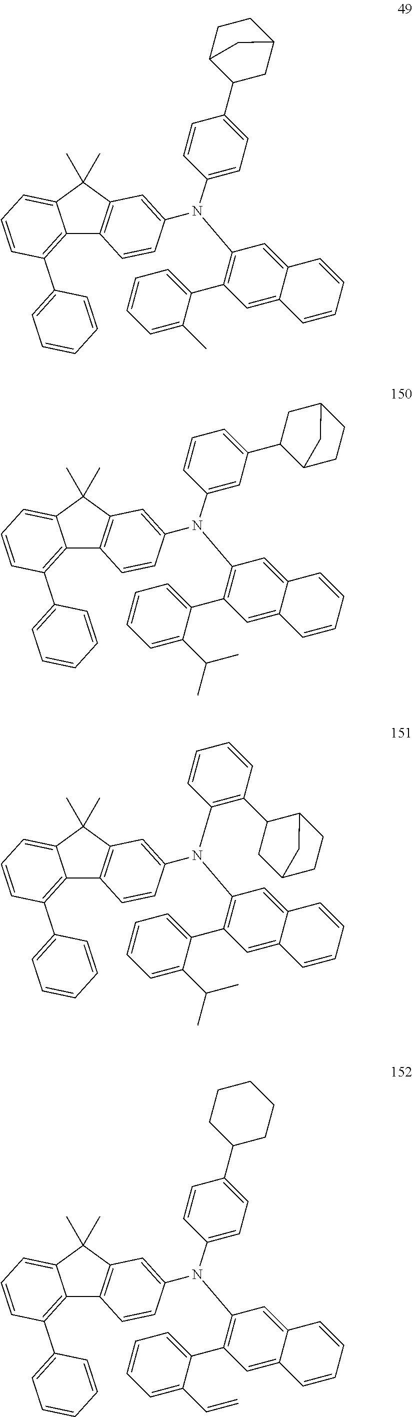 [SMILES: Cc1ccccc1-c1cc2ccccc2cc1N(c1ccc(C2CC3CCC2C3)cc1)c1ccc2c(c1)C(C)(C)c1cccc(-c3ccccc3)c1-2 ; CC(C)c1ccccc1-c1cc2ccccc2cc1N(c1cccc(C2CC3CCC2C3)c1)c1ccc2c(c1)C(C)(C)c1cccc(-c3ccccc3)c1-2 ; CC(C)c1ccccc1-c1cc2ccccc2cc1N(c1ccc2c(c1)C(C)(C)c1cccc(-c3ccccc3)c1-2)c1ccccc1C1CC2CCC1C2 ; C=Cc1ccccc1-c1cc2ccccc2cc1N(c1ccc(C2CCCCC2)cc1)c1ccc2c(c1)C(C)(C)c1cccc(-c3ccccc3)c1-2] 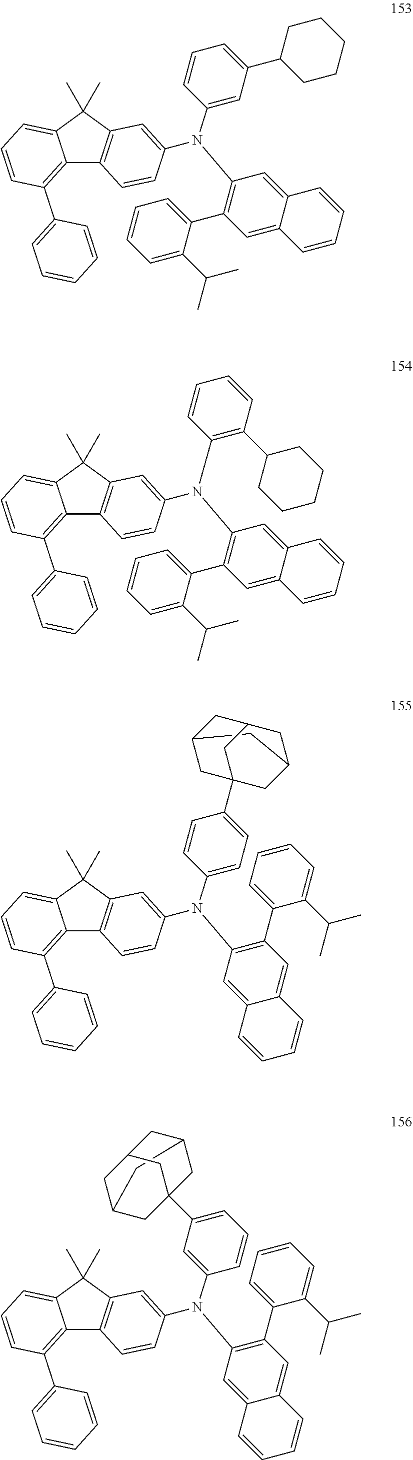 [SMILES: CC(C)c1ccccc1-c1cc2ccccc2cc1N(c1cccc(C2CCCCC2)c1)c1ccc2c(c1)C(C)(C)c1cccc(-c3ccccc3)c1-2 ; CC(C)c1ccccc1-c1cc2ccccc2cc1N(c1ccc2c(c1)C(C)(C)c1cccc(-c3ccccc3)c1-2)c1ccccc1C1CCCCC1 ; CC(C)c1ccccc1-c1cc2ccccc2cc1N(c1ccc(C23CC4CC(CC(C4)C2)C3)cc1)c1ccc2c(c1)C(C)(C)c1cccc(-c3ccccc3)c1-2 ; CC(C)c1ccccc1-c1cc2ccccc2cc1N(c1cccc(C23CC4CC(CC(C4)C2)C3)c1)c1ccc2c(c1)C(C)(C)c1cccc(-c3ccccc3)c1-2] 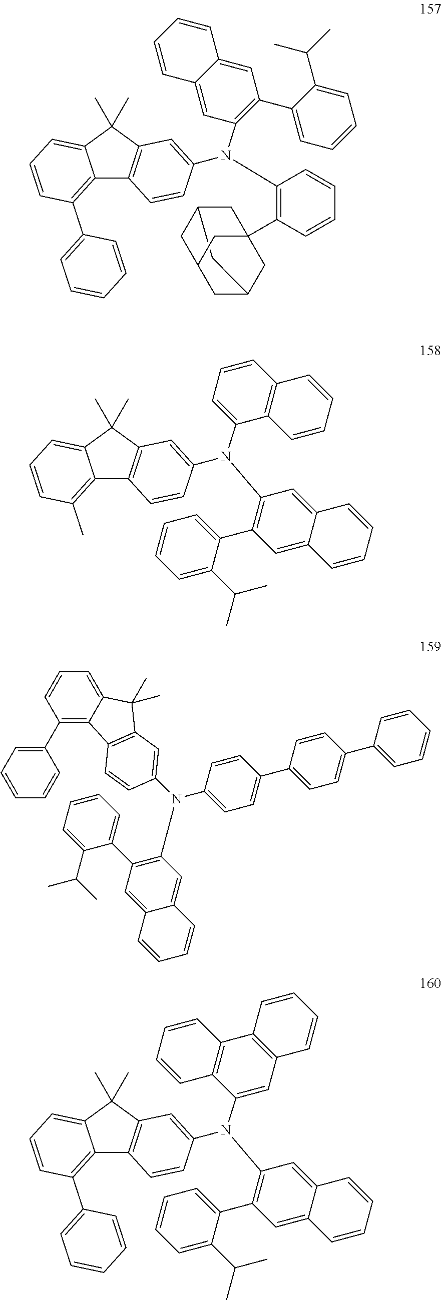 [SMILES: CC(C)c1ccccc1-c1cc2ccccc2cc1N(c1ccc2c(c1)C(C)(C)c1cccc(-c3ccccc3)c1-2)c1ccccc1C12CC3CC(CC(C3)C1)C2 ; Cc1cccc2c1-c1ccc(N(c3cc4ccccc4cc3-c3ccccc3C(C)C)c3cccc4ccccc34)cc1C2(C)C ; CC(C)c1ccccc1-c1cc2ccccc2cc1N(c1ccc(-c2ccc(-c3ccccc3)cc2)cc1)c1ccc2c(c1)C(C)(C)c1cccc(-c3ccccc3)c1-2 ; CC(C)c1ccccc1-c1cc2ccccc2cc1N(c1ccc2c(c1)C(C)(C)c1cccc(-c3ccccc3)c1-2)c1cc2ccccc2c2ccccc12] 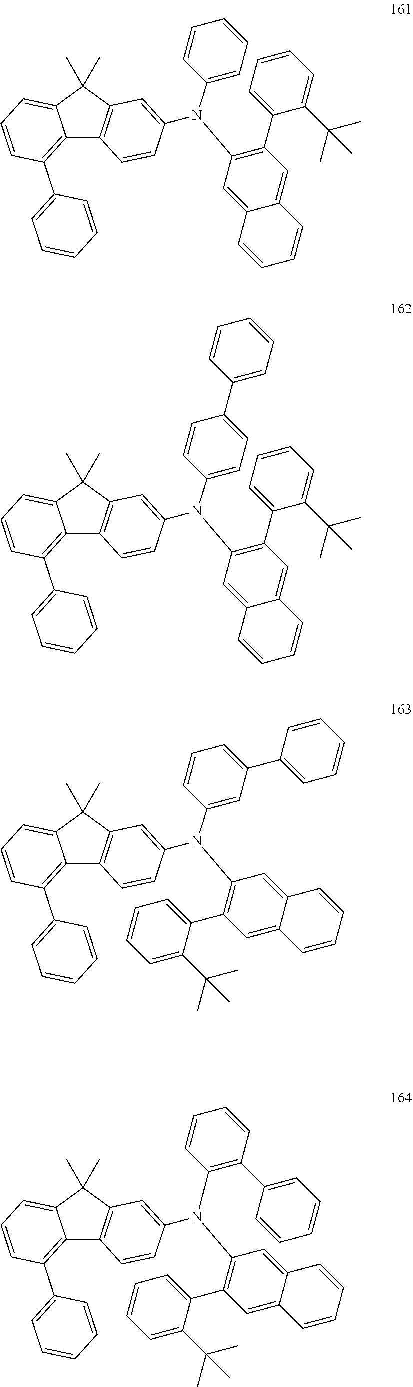 [SMILES: CC(C)(C)c1ccccc1-c1cc2ccccc2cc1N(c1ccccc1)c1ccc2c(c1)C(C)(C)c1cccc(-c3ccccc3)c1-2 ; CC(C)(C)c1ccccc1-c1cc2ccccc2cc1N(c1ccc(-c2ccccc2)cc1)c1ccc2c(c1)C(C)(C)c1cccc(-c3ccccc3)c1-2 ; CC(C)(C)c1ccccc1-c1cc2ccccc2cc1N(c1cccc(-c2ccccc2)c1)c1ccc2c(c1)C(C)(C)c1cccc(-c3ccccc3)c1-2 ; CC(C)(C)c1ccccc1-c1cc2ccccc2cc1N(c1ccc2c(c1)C(C)(C)c1cccc(-c3ccccc3)c1-2)c1ccccc1-c1ccccc1] 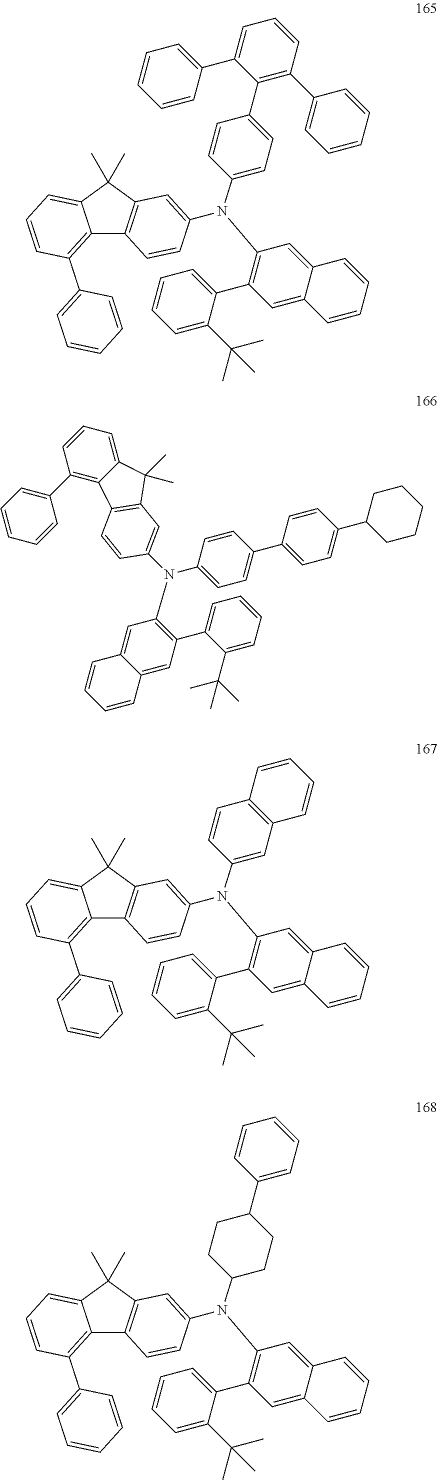 [SMILES: CC(C)(C)c1ccccc1-c1cc2ccccc2cc1N(c1ccc(-c2c(-c3ccccc3)cccc2-c2ccccc2)cc1)c1ccc2c(c1)C(C)(C)c1cccc(-c3ccccc3)c1-2 ; CC(C)(C)c1ccccc1-c1cc2ccccc2cc1N(c1ccc(-c2ccc(C3CCCCC3)cc2)cc1)c1ccc2c(c1)C(C)(C)c1cccc(-c3ccccc3)c1-2 ; CC(C)(C)c1ccccc1-c1cc2ccccc2cc1N(c1ccc2c(c1)C(C)(C)c1cccc(-c3ccccc3)c1-2)c1ccc2ccccc2c1 ; CC(C)(C)c1ccccc1-c1cc2ccccc2cc1N(c1ccc2c(c1)C(C)(C)c1cccc(-c3ccccc3)c1-2)C1CCC(c2ccccc2)CC1] 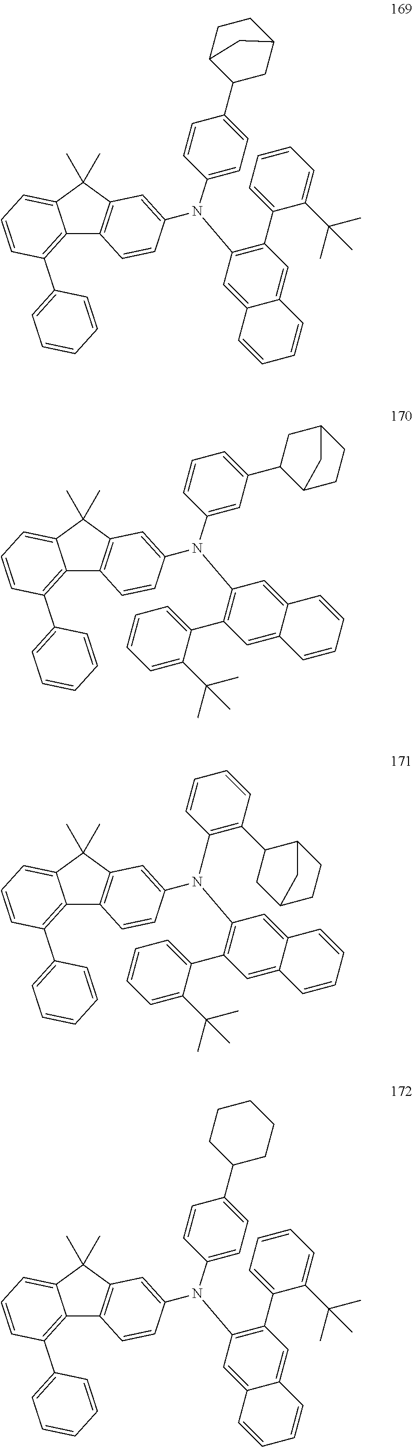 [SMILES: CC(C)(C)c1ccccc1-c1cc2ccccc2cc1N(c1ccc(C2CC3CCC2C3)cc1)c1ccc2c(c1)C(C)(C)c1cccc(-c3ccccc3)c1-2 ; CC(C)(C)c1ccccc1-c1cc2ccccc2cc1N(c1cccc(C2CC3CCC2C3)c1)c1ccc2c(c1)C(C)(C)c1cccc(-c3ccccc3)c1-2 ; CC(C)(C)c1ccccc1-c1cc2ccccc2cc1N(c1ccc2c(c1)C(C)(C)c1cccc(-c3ccccc3)c1-2)c1ccccc1C1CC2CCC1C2 ; CC(C)(C)c1ccccc1-c1cc2ccccc2cc1N(c1ccc(C2CCCCC2)cc1)c1ccc2c(c1)C(C)(C)c1cccc(-c3ccccc3)c1-2] 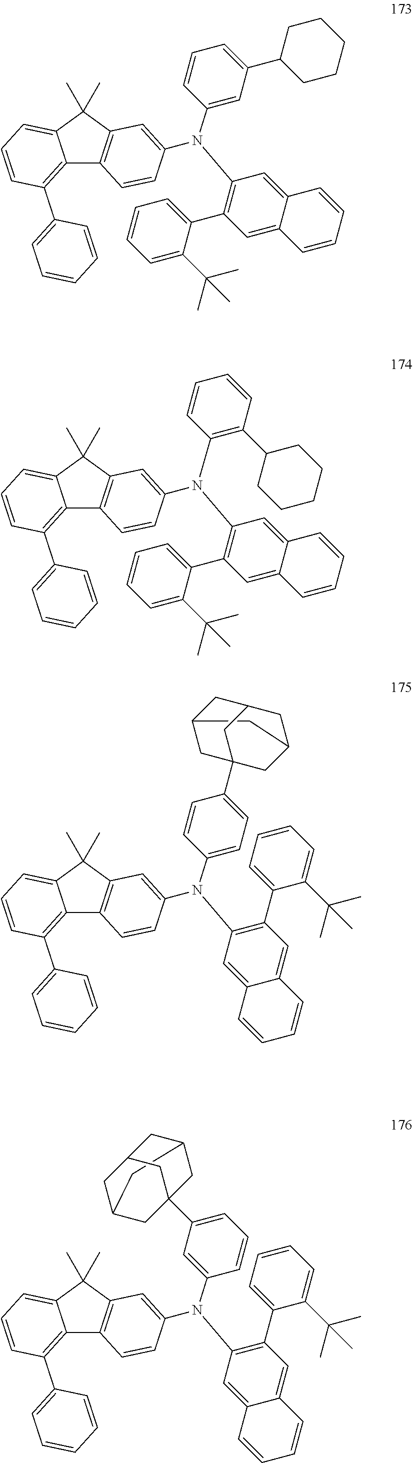 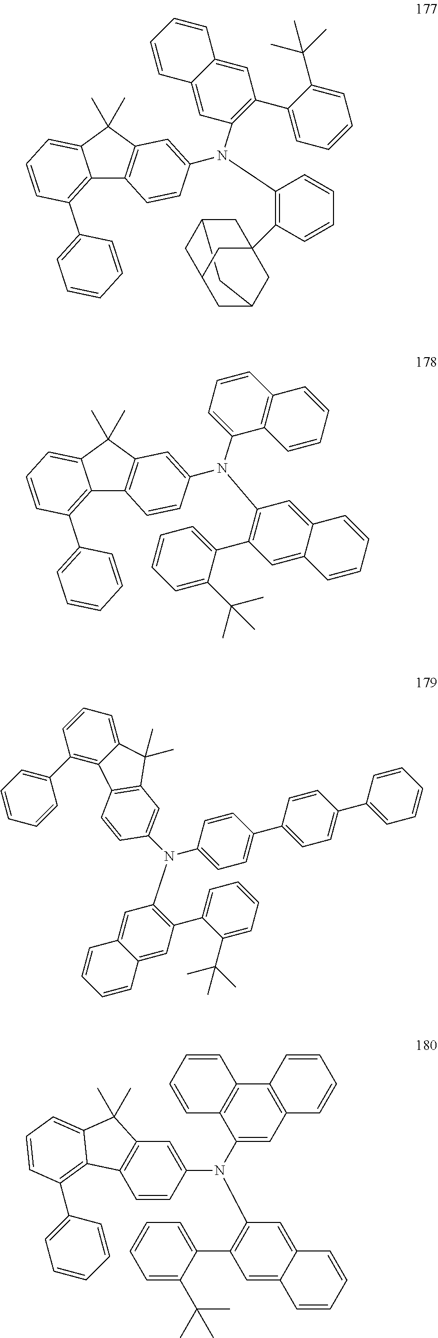 [SMILES: CC(C)(C)c1ccccc1-c1cc2ccccc2cc1N(c1ccc2c(c1)C(C)(C)c1cccc(-c3ccccc3)c1-2)c1ccccc1C12CC3CC(CC(C3)C1)C2 ; CC(C)(C)c1ccccc1-c1cc2ccccc2cc1N(c1ccc2c(c1)C(C)(C)c1cccc(-c3ccccc3)c1-2)c1cccc2ccccc12 ; CC(C)(C)c1ccccc1-c1cc2ccccc2cc1N(c1ccc(-c2ccc(-c3ccccc3)cc2)cc1)c1ccc2c(c1)C(C)(C)c1cccc(-c3ccccc3)c1-2 ; CC(C)(C)c1ccccc1-c1cc2ccccc2cc1N(c1ccc2c(c1)C(C)(C)c1cccc(-c3ccccc3)c1-2)c1cc2ccccc2c2ccccc12] 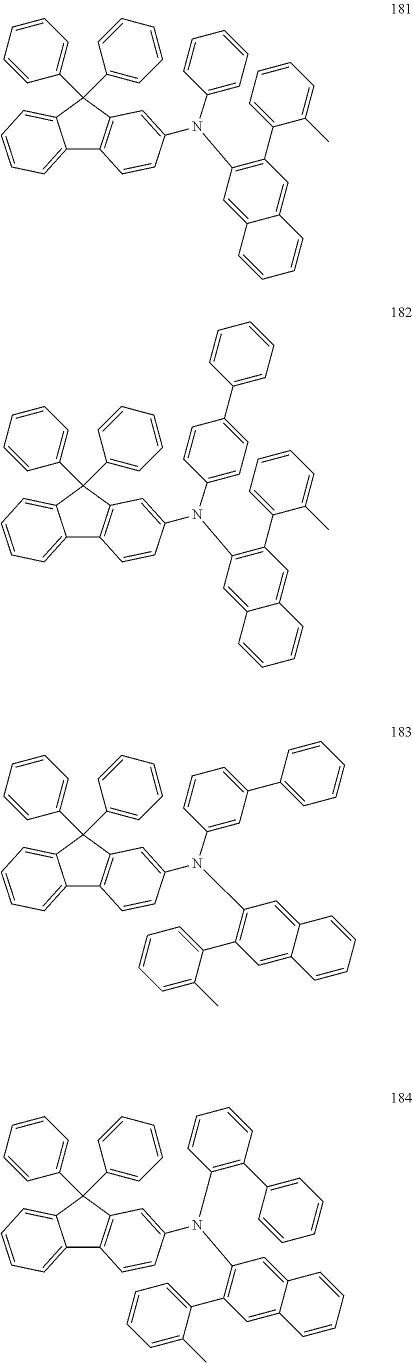 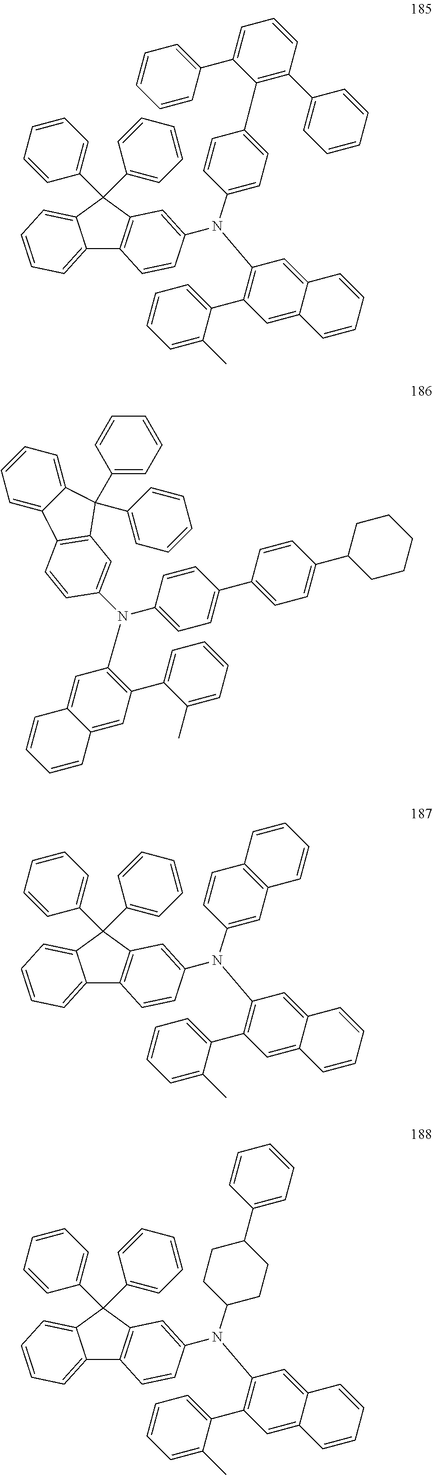 [SMILES: Cc1ccccc1-c1cc2ccccc2cc1N(c1ccc(-c2c(-c3ccccc3)cccc2-c2ccccc2)cc1)c1ccc2c(c1)C(c1ccccc1)(c1ccccc1)c1ccccc1-2 ; Cc1ccccc1-c1cc2ccccc2cc1N(c1ccc(-c2ccc(C3CCCCC3)cc2)cc1)c1ccc2c(c1)C(c1ccccc1)(c1ccccc1)c1ccccc1-2 ; Cc1ccccc1-c1cc2ccccc2cc1N(c1ccc2c(c1)C(c1ccccc1)(c1ccccc1)c1ccccc1-2)c1ccc2ccccc2c1 ; Cc1ccccc1-c1cc2ccccc2cc1N(c1ccc2c(c1)C(c1ccccc1)(c1ccccc1)c1ccccc1-2)C1CCC(c2ccccc2)CC1] 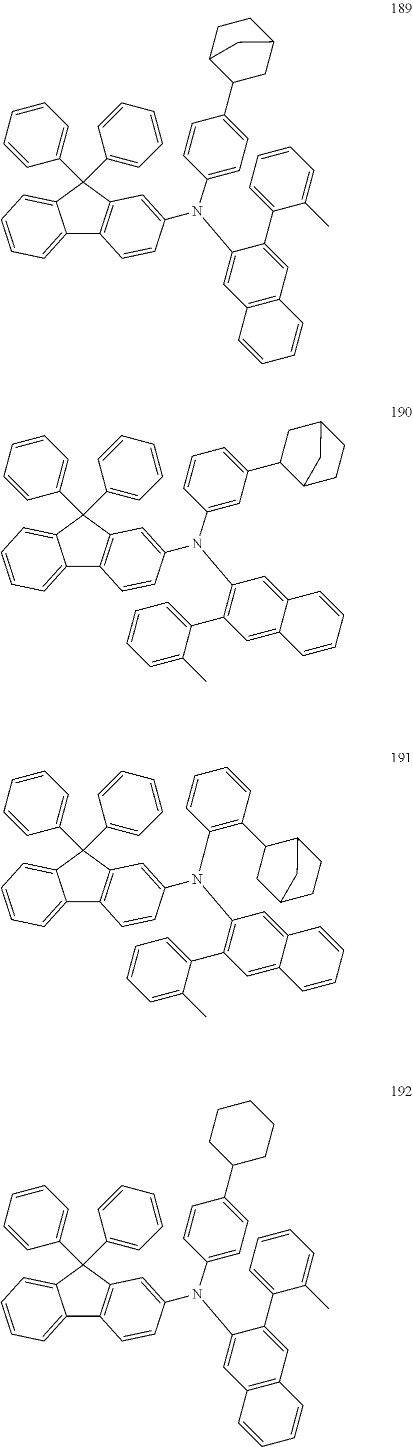 [SMILES: Cc1ccccc1-c1cc2ccccc2cc1N(c1ccc(C2CC3CCC2C3)cc1)c1ccc2c(c1)C(c1ccccc1)(c1ccccc1)c1ccccc1-2 ; Cc1ccccc1-c1cc2ccccc2cc1N(c1cccc(C2CC3CCC2C3)c1)c1ccc2c(c1)C(c1ccccc1)(c1ccccc1)c1ccccc1-2 ; Cc1ccccc1-c1cc2ccccc2cc1N(c1ccc2c(c1)C(c1ccccc1)(c1ccccc1)c1ccccc1-2)c1ccccc1C1CC2CCC1C2 ; Cc1ccccc1-c1cc2ccccc2cc1N(c1ccc(C2CCCCC2)cc1)c1ccc2c(c1)C(c1ccccc1)(c1ccccc1)c1ccccc1-2] 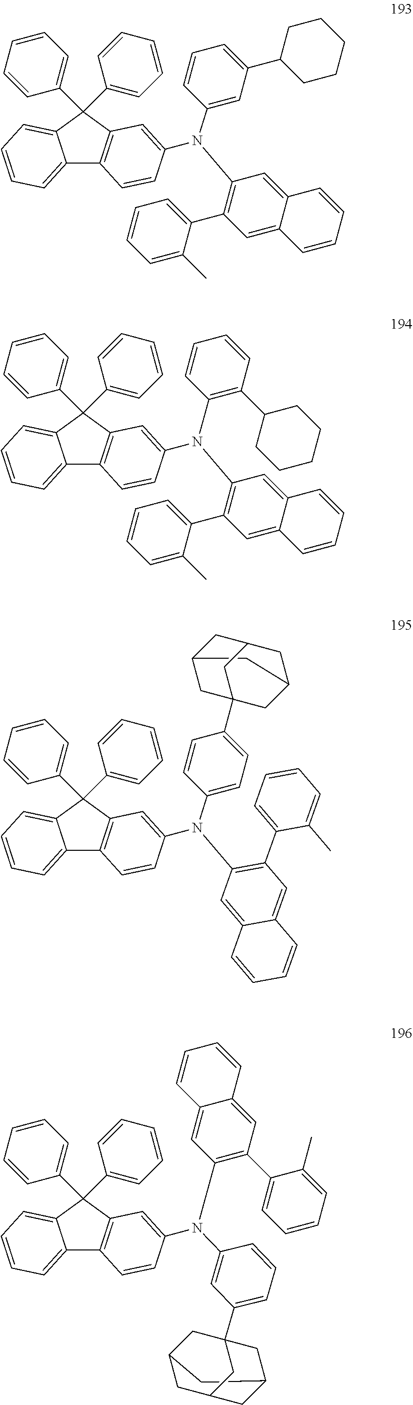 [SMILES: Cc1ccccc1-c1cc2ccccc2cc1N(c1cccc(C2CCCCC2)c1)c1ccc2c(c1)C(c1ccccc1)(c1ccccc1)c1ccccc1-2 ; Cc1ccccc1-c1cc2ccccc2cc1N(c1ccc2c(c1)C(c1ccccc1)(c1ccccc1)c1ccccc1-2)c1ccccc1C1CCCCC1 ; Cc1ccccc1-c1cc2ccccc2cc1N(c1ccc(C23CC4CC(CC(C4)C2)C3)cc1)c1ccc2c(c1)C(c1ccccc1)(c1ccccc1)c1ccccc1-2 ; Cc1ccccc1-c1cc2ccccc2cc1N(c1cccc(C23CC4CC(CC(C4)C2)C3)c1)c1ccc2c(c1)C(c1ccccc1)(c1ccccc1)c1ccccc1-2] 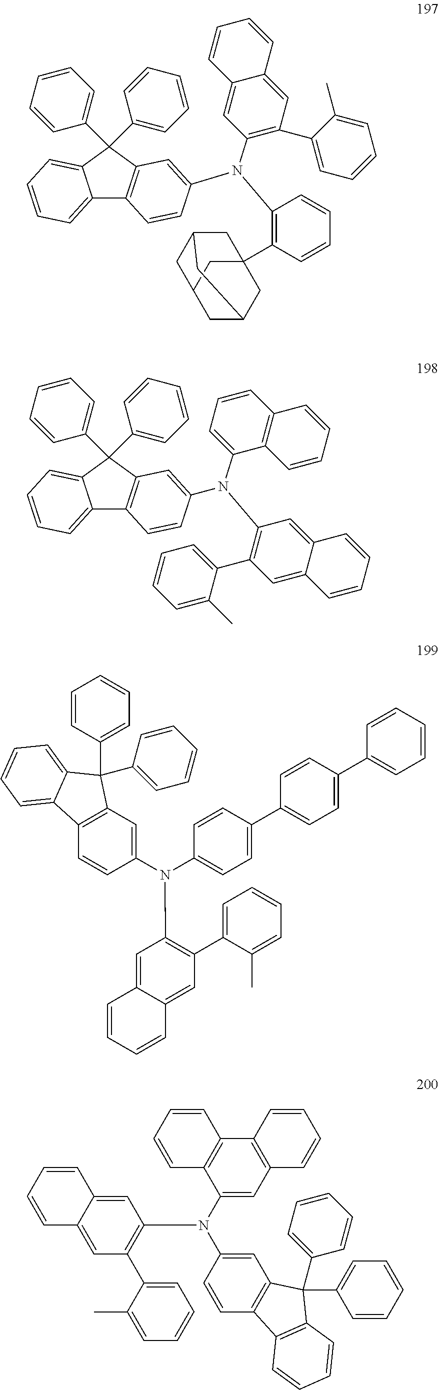 [SMILES: Cc1ccccc1-c1cc2ccccc2cc1N(c1ccc2c(c1)C(c1ccccc1)(c1ccccc1)c1ccccc1-2)c1ccccc1C12CC3CC(CC(C3)C1)C2 ; Cc1ccccc1-c1cc2ccccc2cc1N(c1ccc2c(c1)C(c1ccccc1)(c1ccccc1)c1ccccc1-2)c1cccc2ccccc12 ; Cc1ccccc1-c1cc2ccccc2cc1N(c1ccc(-c2ccc(-c3ccccc3)cc2)cc1)c1ccc2c(c1)C(c1ccccc1)(c1ccccc1)c1ccccc1-2 ; Cc1ccccc1-c1cc2ccccc2cc1N(c1ccc2c(c1)C(c1ccccc1)(c1ccccc1)c1ccccc1-2)c1cc2ccccc2c2ccccc12] 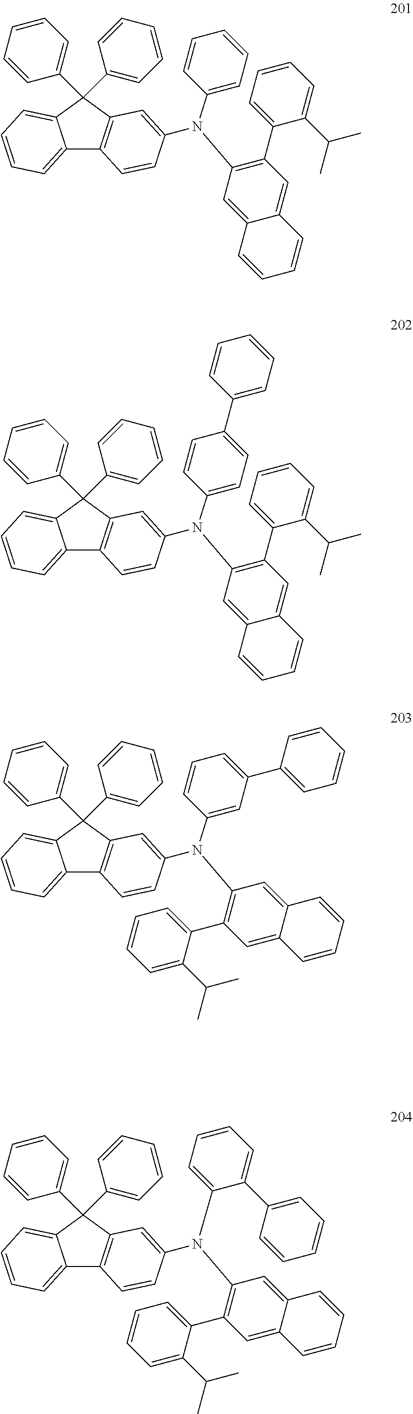 [SMILES: CC(C)c1ccccc1-c1cc2ccccc2cc1N(c1ccccc1)c1ccc2c(c1)C(c1ccccc1)(c1ccccc1)c1ccccc1-2 ; CC(C)c1ccccc1-c1cc2ccccc2cc1N(c1ccc(-c2ccccc2)cc1)c1ccc2c(c1)C(c1ccccc1)(c1ccccc1)c1ccccc1-2 ; CC(C)c1ccccc1-c1cc2ccccc2cc1N(c1cccc(-c2ccccc2)c1)c1ccc2c(c1)C(c1ccccc1)(c1ccccc1)c1ccccc1-2 ; CC(C)c1ccccc1-c1cc2ccccc2cc1N(c1ccc2c(c1)C(c1ccccc1)(c1ccccc1)c1ccccc1-2)c1ccccc1-c1ccccc1] 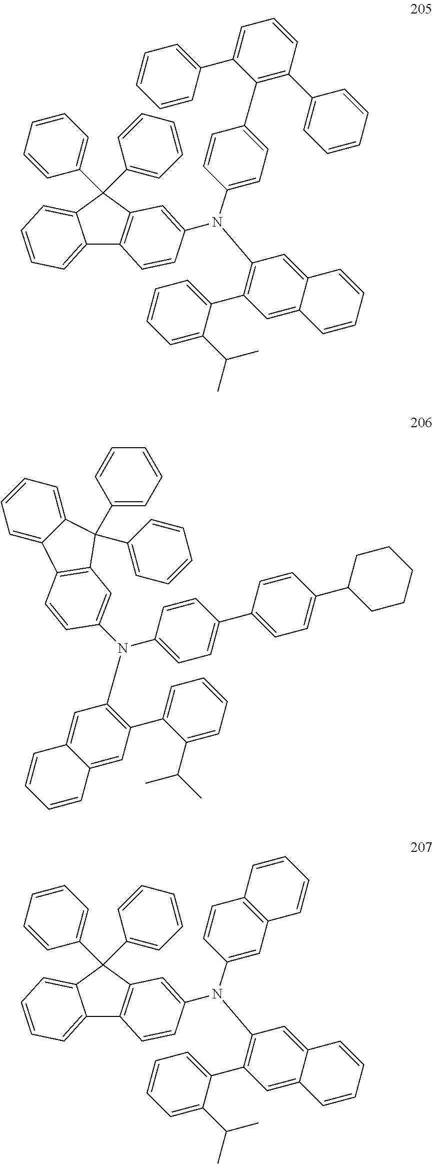 [SMILES: CC(C)c1ccccc1-c1cc2ccccc2cc1N(c1ccc(-c2c(-c3ccccc3)cccc2-c2ccccc2)cc1)c1ccc2c(c1)C(c1ccccc1)(c1ccccc1)c1ccccc1-2 ; CC(C)c1ccccc1-c1cc2ccccc2cc1N(c1ccc(-c2ccc(C3CCCCC3)cc2)cc1)c1ccc2c(c1)C(c1ccccc1)(c1ccccc1)c1ccccc1-2 ; CC(C)c1ccccc1-c1cc2ccccc2cc1N(c1ccc2c(c1)C(c1ccccc1)(c1ccccc1)c1ccccc1-2)c1ccc2ccccc2c1] 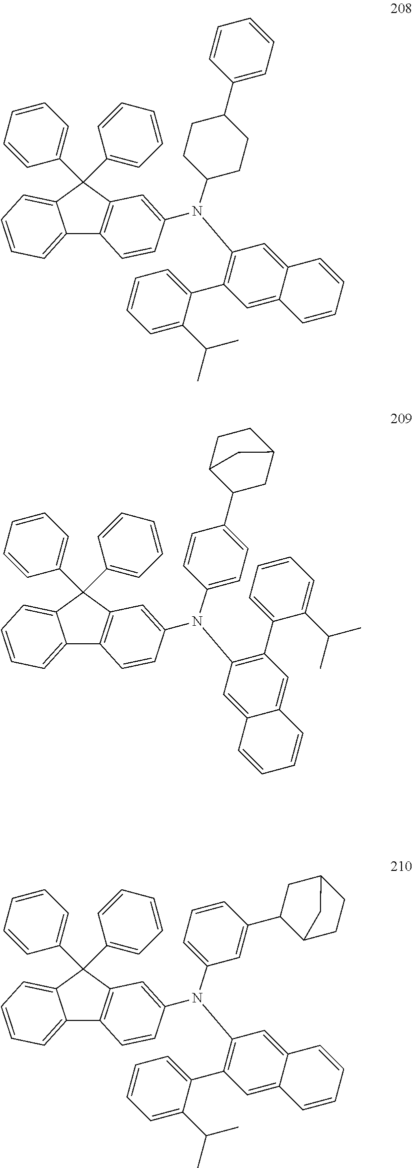 [SMILES: CC(C)c1ccccc1-c1cc2ccccc2cc1N(c1ccc2c(c1)C(c1ccccc1)(c1ccccc1)c1ccccc1-2)C1CCC(c2ccccc2)CC1 ; CC(C)c1ccccc1-c1cc2ccccc2cc1N(c1ccc(C2CC3CCC2C3)cc1)c1ccc2c(c1)C(c1ccccc1)(c1ccccc1)c1ccccc1-2 ; CC(C)c1ccccc1-c1cc2ccccc2cc1N(c1cccc(C2CC3CCC2C3)c1)c1ccc2c(c1)C(c1ccccc1)(c1ccccc1)c1ccccc1-2] 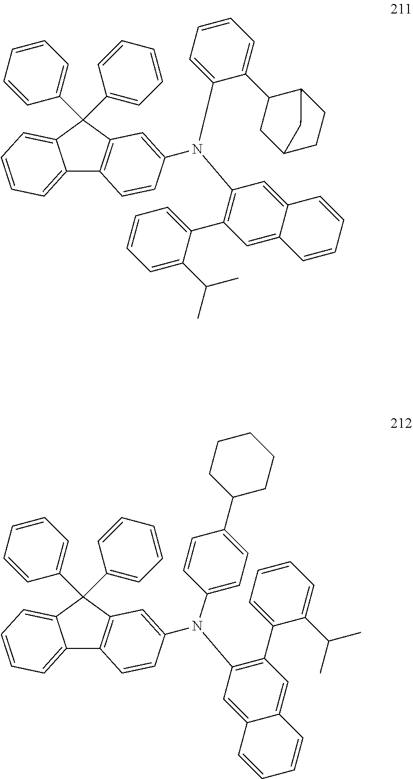 [SMILES: CC(C)c1ccccc1-c1cc2ccccc2cc1N(c1ccc2c(c1)C(c1ccccc1)(c1ccccc1)c1ccccc1-2)c1ccccc1C1CC2CCC1C2 ; CC(C)c1ccccc1-c1cc2ccccc2cc1N(c1ccc(C2CCCCC2)cc1)c1ccc2c(c1)C(c1ccccc1)(c1ccccc1)c1ccccc1-2] 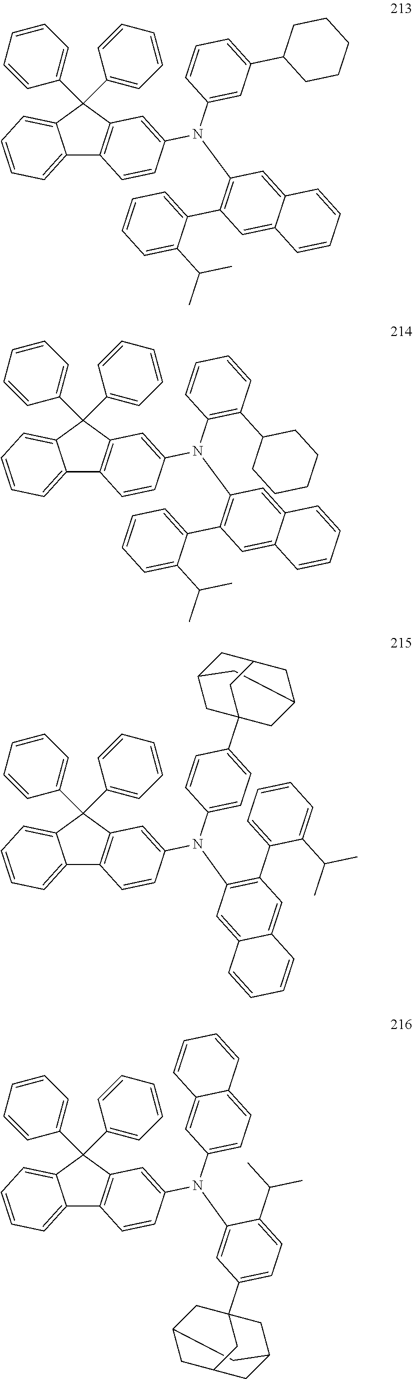 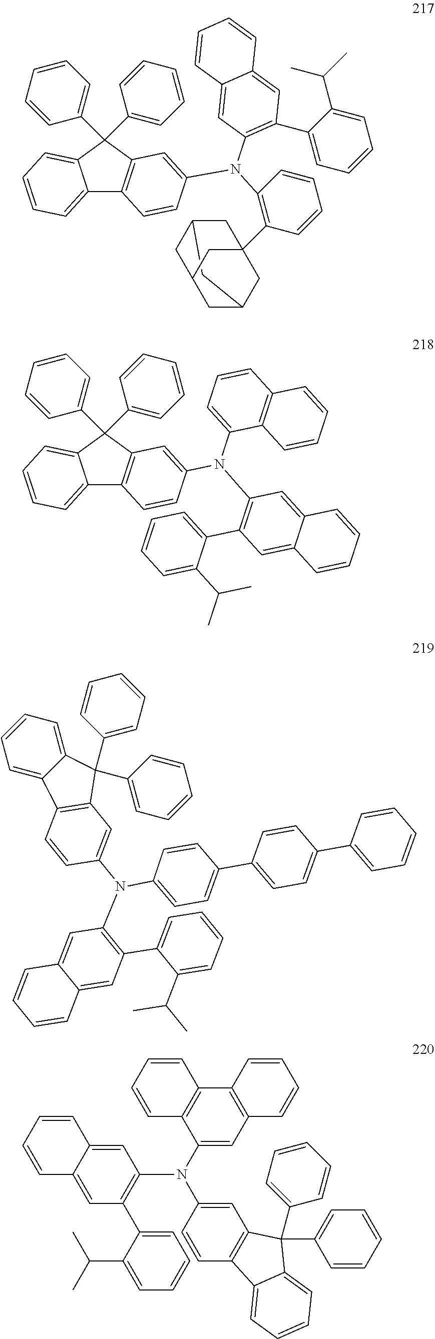 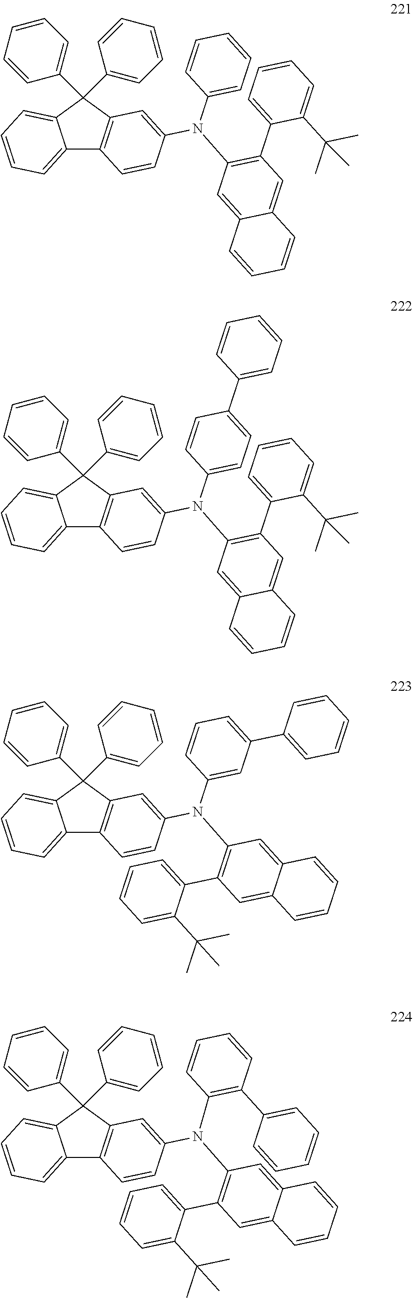 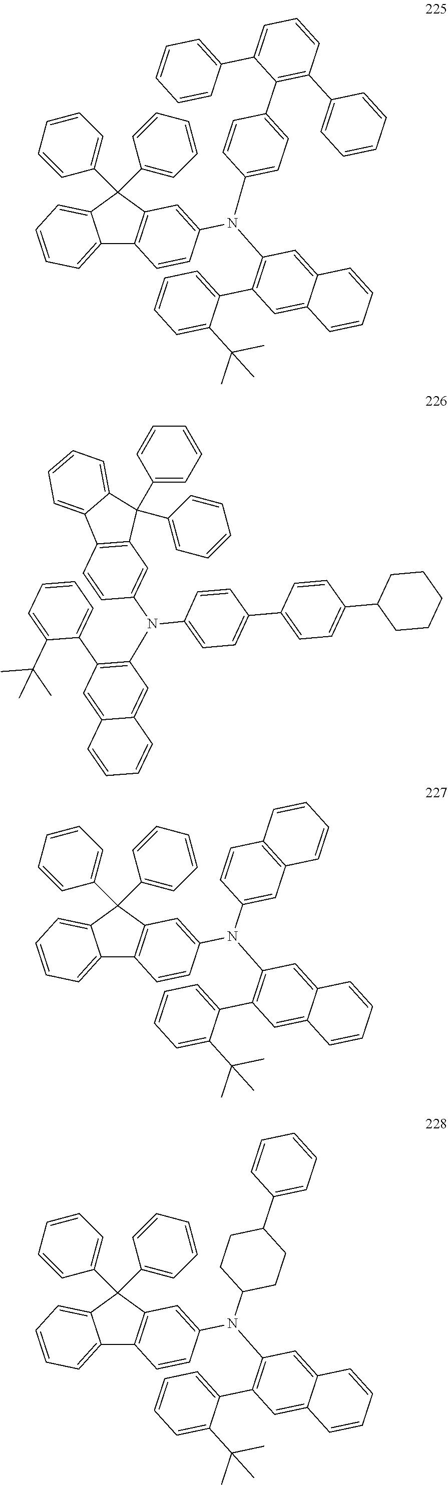 [SMILES: CC(C)(C)c1ccccc1-c1cc2ccccc2cc1N(c1ccc(-c2c(-c3ccccc3)cccc2-c2ccccc2)cc1)c1ccc2c(c1)C(c1ccccc1)(c1ccccc1)c1ccccc1-2 ; CC(C)(C)c1ccccc1-c1cc2ccccc2cc1N(c1ccc(-c2ccc(C3CCCCC3)cc2)cc1)c1ccc2c(c1)C(c1ccccc1)(c1ccccc1)c1ccccc1-2 ; CC(C)(C)c1ccccc1-c1cc2ccccc2cc1N(c1ccc2c(c1)C(c1ccccc1)(c1ccccc1)c1ccccc1-2)c1ccc2ccccc2c1 ; CC(C)(C)c1ccccc1-c1cc2ccccc2cc1N(c1ccc2c(c1)C(c1ccccc1)(c1ccccc1)c1ccccc1-2)C1CCC(c2ccccc2)CC1] 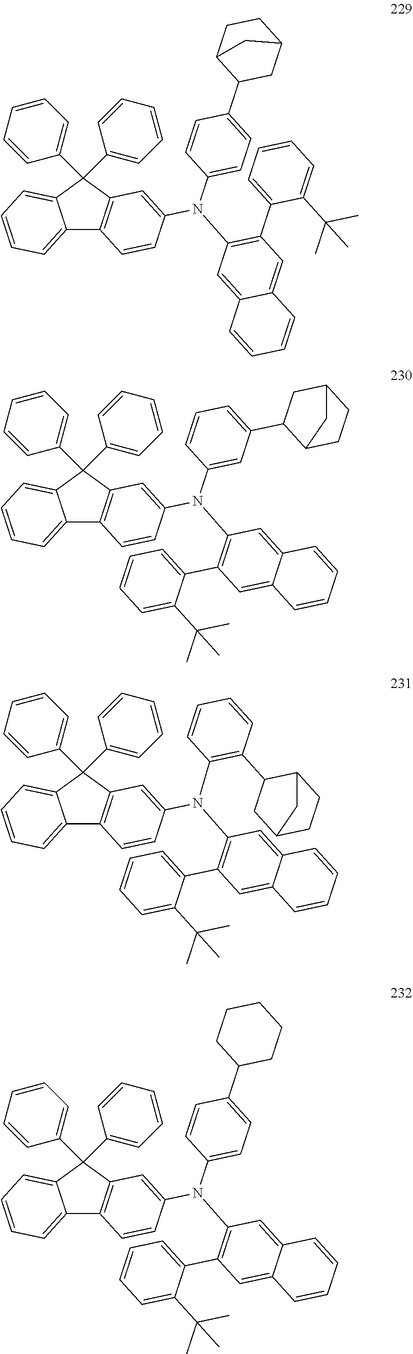 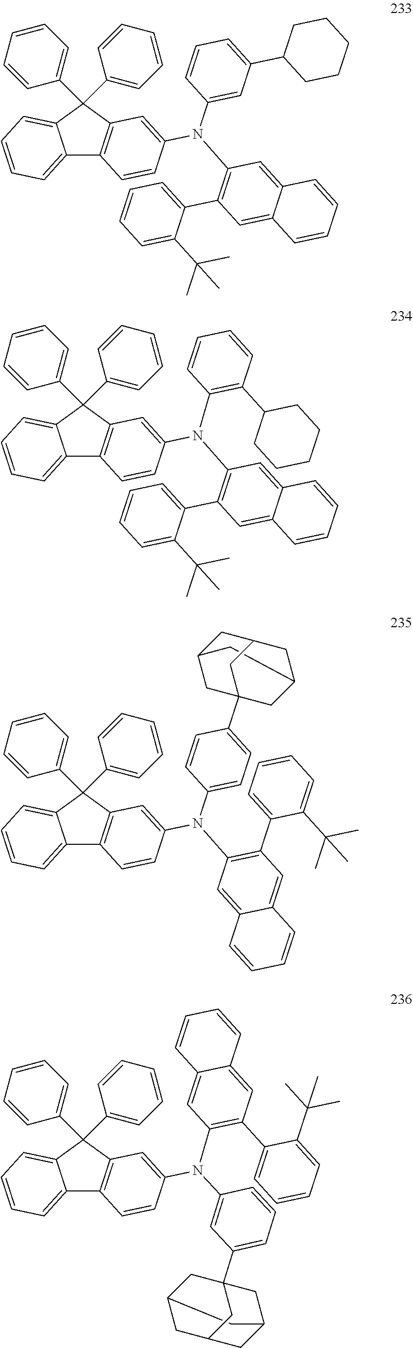 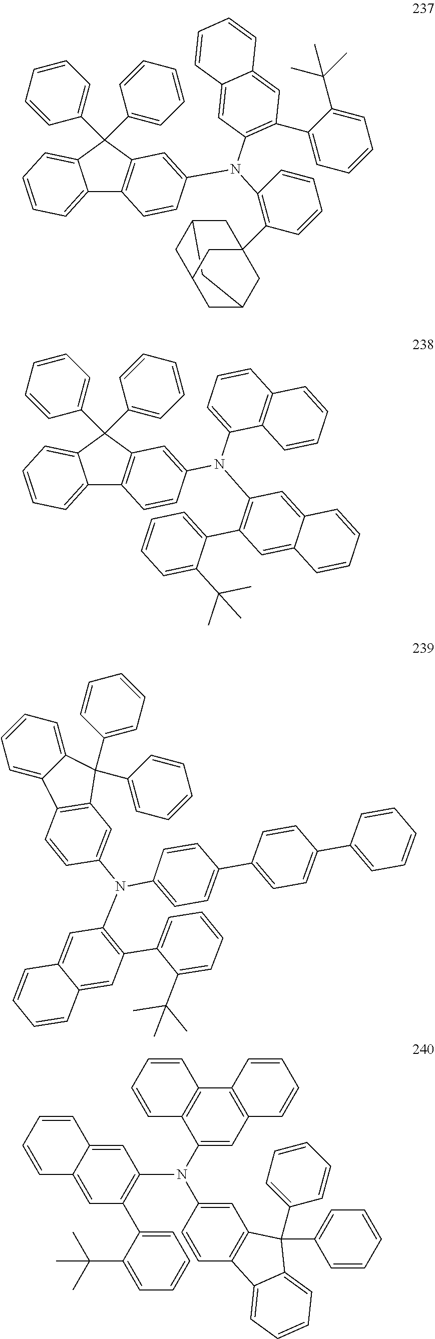 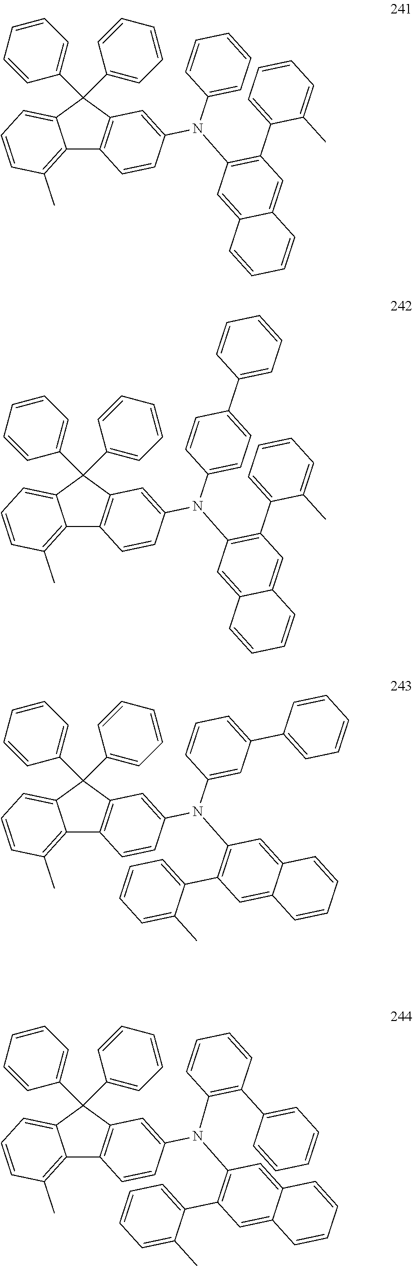 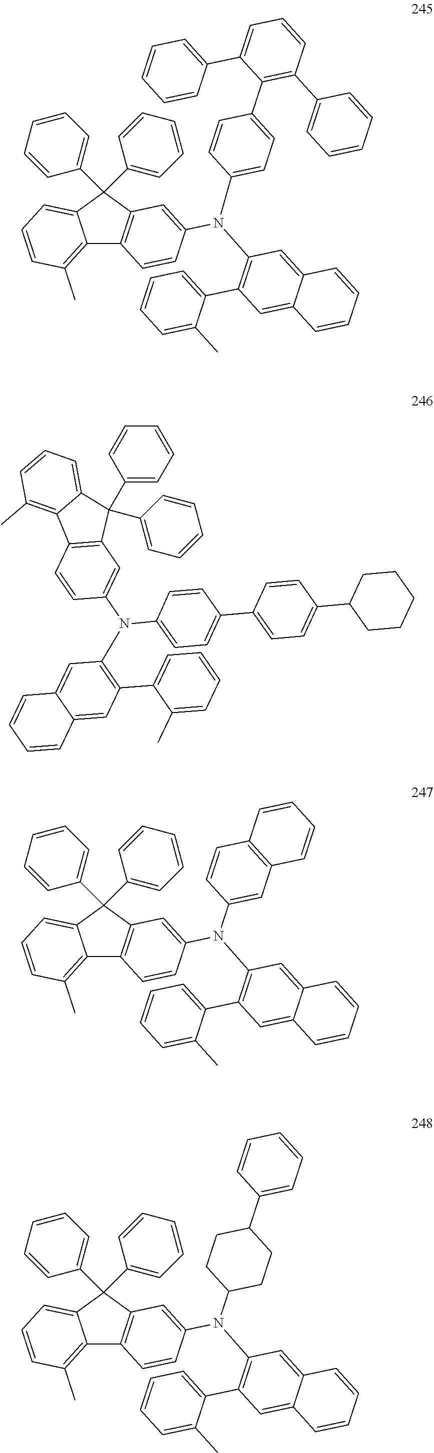 [SMILES: Cc1ccccc1-c1cc2ccccc2cc1N(c1ccc(-c2c(-c3ccccc3)cccc2-c2ccccc2)cc1)c1ccc2c(c1)C(c1ccccc1)(c1ccccc1)c1cccc(C)c1-2 ; Cc1ccccc1-c1cc2ccccc2cc1N(c1ccc(-c2ccc(C3CCCCC3)cc2)cc1)c1ccc2c(c1)C(c1ccccc1)(c1ccccc1)c1cccc(C)c1-2 ; Cc1ccccc1-c1cc2ccccc2cc1N(c1ccc2c(c1)C(c1ccccc1)(c1ccccc1)c1cccc(C)c1-2)c1ccc2ccccc2c1 ; Cc1ccccc1-c1cc2ccccc2cc1N(c1ccc2c(c1)C(c1ccccc1)(c1ccccc1)c1cccc(C)c1-2)C1CCC(c2ccccc2)CC1] 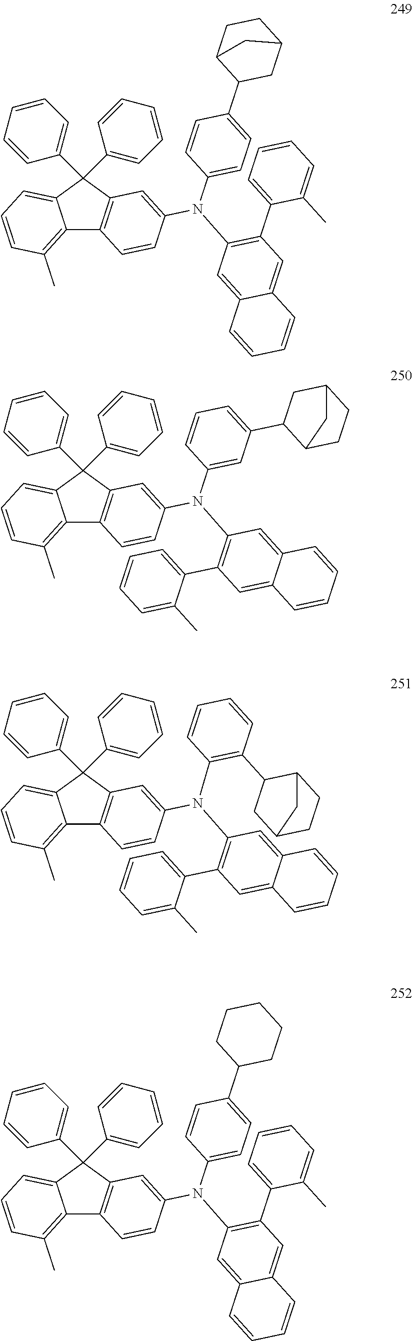 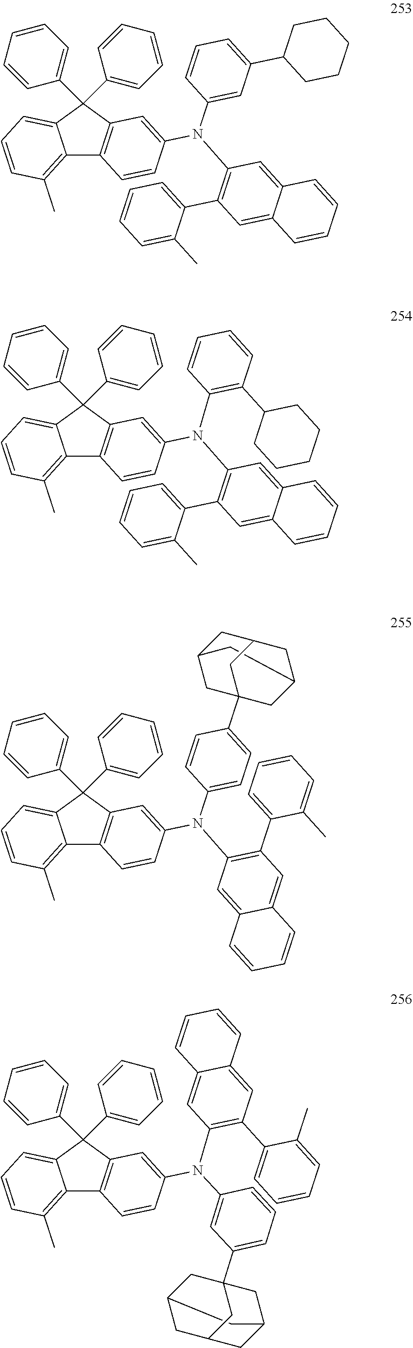 [SMILES: Cc1ccccc1-c1cc2ccccc2cc1N(c1cccc(C2CCCCC2)c1)c1ccc2c(c1)C(c1ccccc1)(c1ccccc1)c1cccc(C)c1-2 ; Cc1ccccc1-c1cc2ccccc2cc1N(c1ccc2c(c1)C(c1ccccc1)(c1ccccc1)c1cccc(C)c1-2)c1ccccc1C1CCCCC1 ; Cc1ccccc1-c1cc2ccccc2cc1N(c1ccc(C23CC4CC(CC(C4)C2)C3)cc1)c1ccc2c(c1)C(c1ccccc1)(c1ccccc1)c1cccc(C)c1-2 ; Cc1ccccc1-c1cc2ccccc2cc1N(c1cccc(C23CC4CC(CC(C4)C2)C3)c1)c1ccc2c(c1)C(c1ccccc1)(c1ccccc1)c1cccc(C)c1-2] 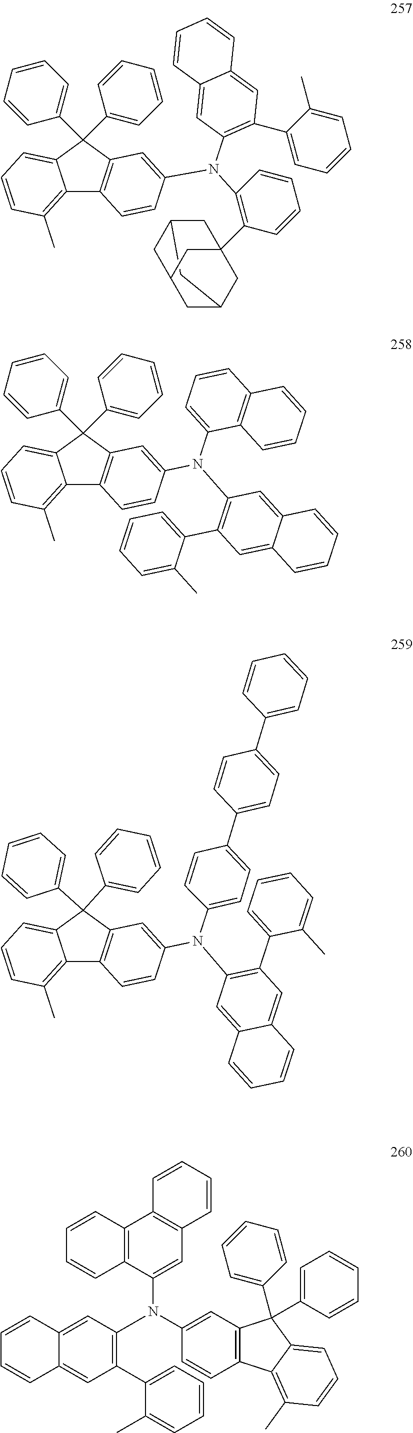 [SMILES: Cc1ccccc1-c1cc2ccccc2cc1N(c1ccc2c(c1)C(c1ccccc1)(c1ccccc1)c1cccc(C)c1-2)c1ccccc1C12CC3CC(CC(C3)C1)C2 ; Cc1ccccc1-c1cc2ccccc2cc1N(c1ccc2c(c1)C(c1ccccc1)(c1ccccc1)c1cccc(C)c1-2)c1cccc2ccccc12 ; Cc1ccccc1-c1cc2ccccc2cc1N(c1ccc(-c2ccc(-c3ccccc3)cc2)cc1)c1ccc2c(c1)C(c1ccccc1)(c1ccccc1)c1cccc(C)c1-2 ; Cc1ccccc1-c1cc2ccccc2cc1N(c1ccc2c(c1)C(c1ccccc1)(c1ccccc1)c1cccc(C)c1-2)c1cc2ccccc2c2ccccc12] 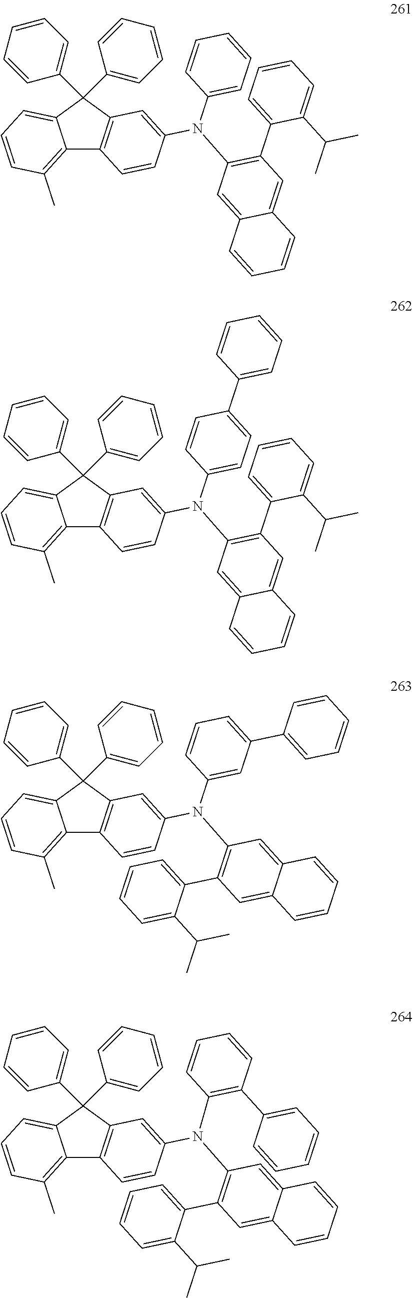 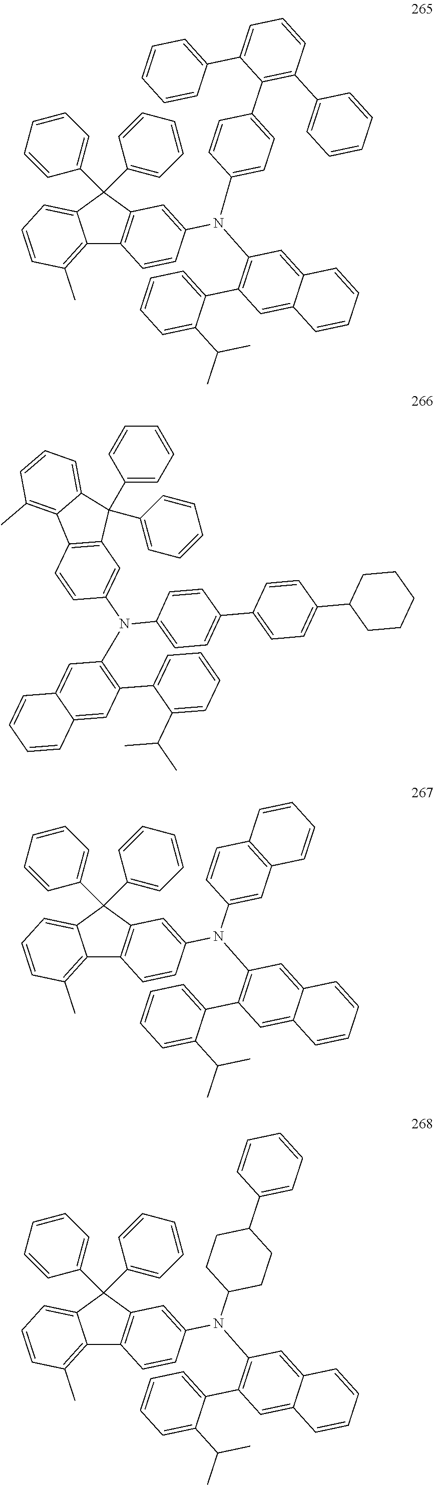 [SMILES: Cc1cccc2c1-c1ccc(N(c3ccc(-c4c(-c5ccccc5)cccc4-c4ccccc4)cc3)c3cc4ccccc4cc3-c3ccccc3C(C)C)cc1C2(c1ccccc1)c1ccccc1 ; Cc1cccc2c1-c1ccc(N(c3ccc(-c4ccc(C5CCCCC5)cc4)cc3)c3cc4ccccc4cc3-c3ccccc3C(C)C)cc1C2(c1ccccc1)c1ccccc1 ; Cc1cccc2c1-c1ccc(N(c3ccc4ccccc4c3)c3cc4ccccc4cc3-c3ccccc3C(C)C)cc1C2(c1ccccc1)c1ccccc1 ; Cc1cccc2c1-c1ccc(N(c3cc4ccccc4cc3-c3ccccc3C(C)C)C3CCC(c4ccccc4)CC3)cc1C2(c1ccccc1)c1ccccc1] 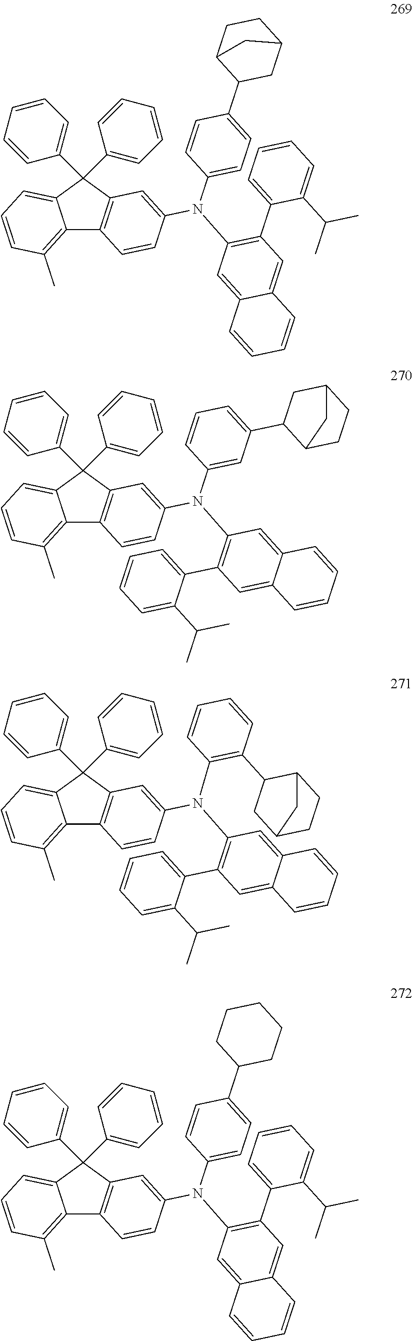 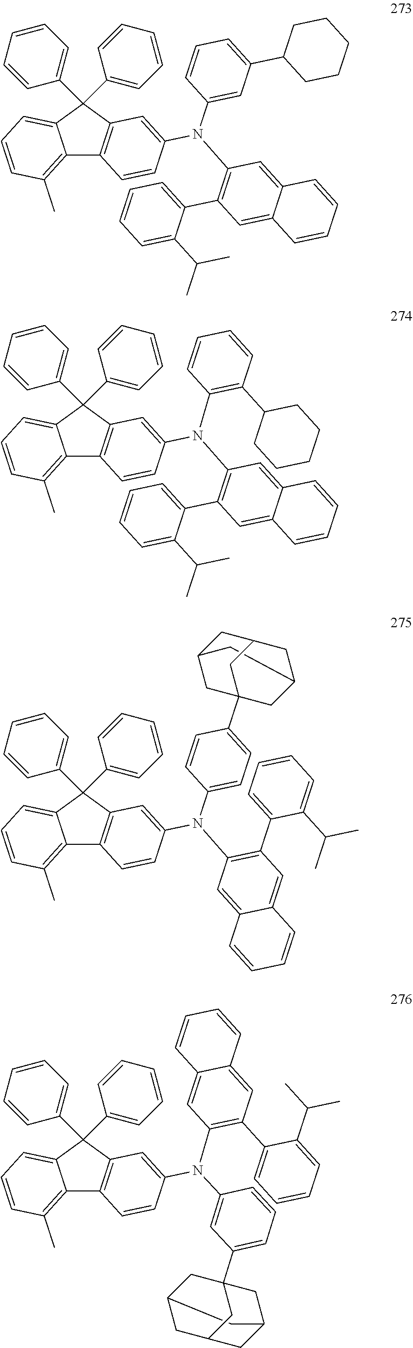 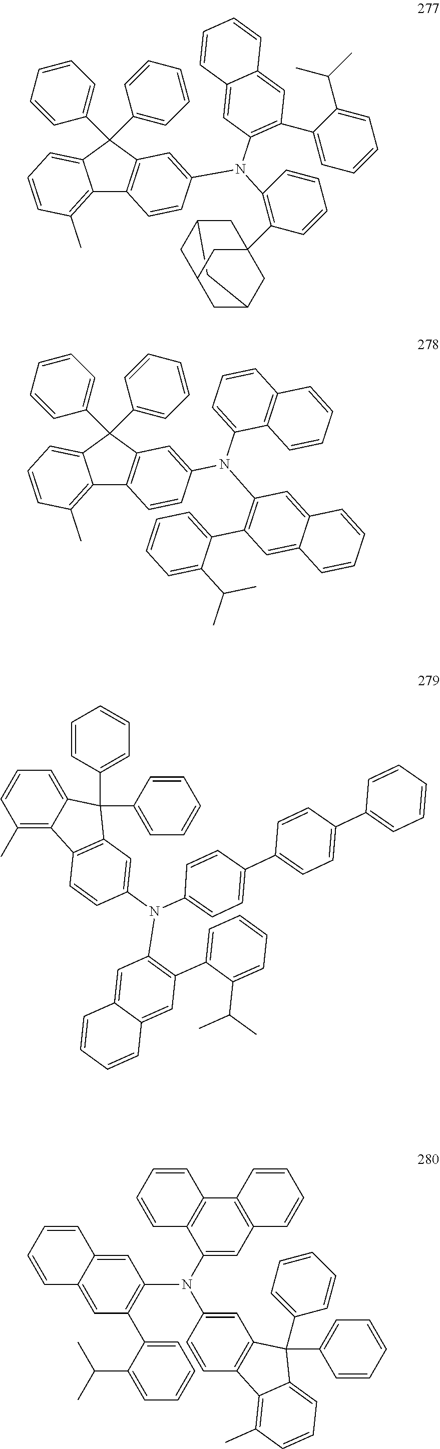 [SMILES: Cc1cccc2c1-c1ccc(N(c3cc4ccccc4cc3-c3ccccc3C(C)C)c3ccccc3C34CC5CC(CC(C5)C3)C4)cc1C2(c1ccccc1)c1ccccc1 ; Cc1cccc2c1-c1ccc(N(c3cc4ccccc4cc3-c3ccccc3C(C)C)c3cccc4ccccc34)cc1C2(c1ccccc1)c1ccccc1 ; Cc1cccc2c1-c1ccc(N(c3ccc(-c4ccc(-c5ccccc5)cc4)cc3)c3cc4ccccc4cc3-c3ccccc3C(C)C)cc1C2(c1ccccc1)c1ccccc1 ; Cc1cccc2c1-c1ccc(N(c3cc4ccccc4cc3-c3ccccc3C(C)C)c3cc4ccccc4c4ccccc34)cc1C2(c1ccccc1)c1ccccc1] 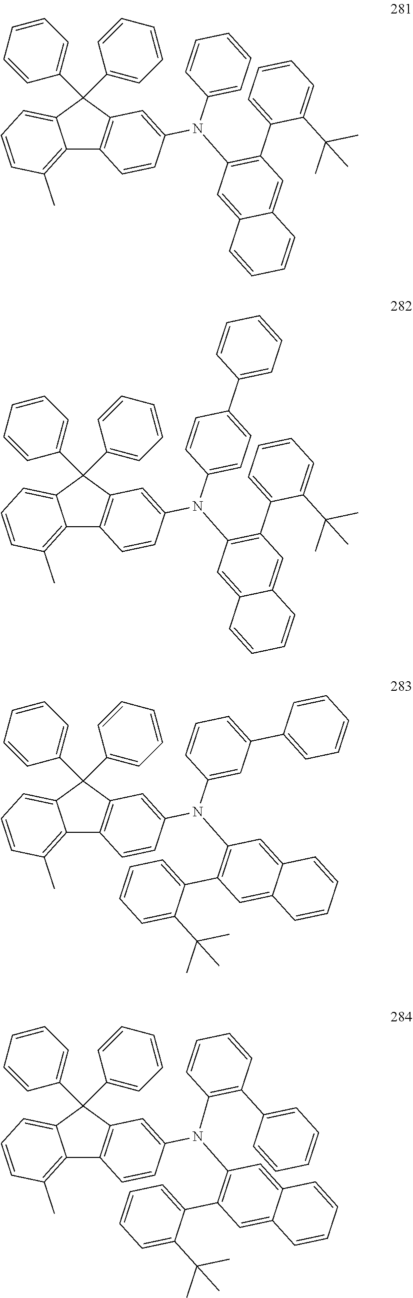 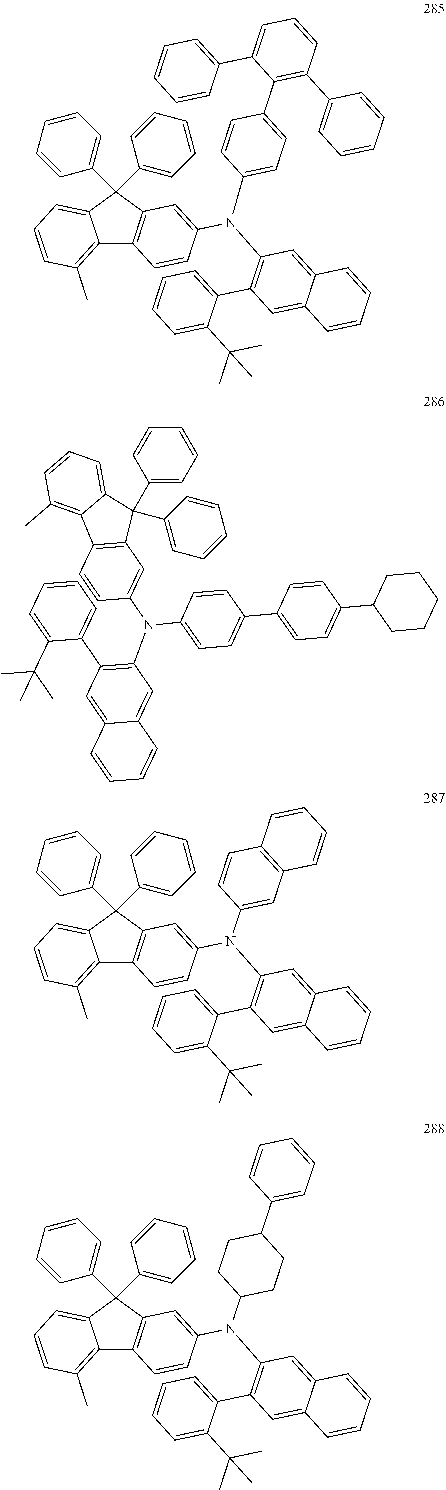 [SMILES: Cc1cccc2c1-c1ccc(N(c3ccc(-c4c(-c5ccccc5)cccc4-c4ccccc4)cc3)c3cc4ccccc4cc3-c3ccccc3C(C)(C)C)cc1C2(c1ccccc1)c1ccccc1 ; Cc1cccc2c1-c1ccc(N(c3ccc(-c4ccc(C5CCCCC5)cc4)cc3)c3cc4ccccc4cc3-c3ccccc3C(C)(C)C)cc1C2(c1ccccc1)c1ccccc1 ; Cc1cccc2c1-c1ccc(N(c3ccc4ccccc4c3)c3cc4ccccc4cc3-c3ccccc3C(C)(C)C)cc1C2(c1ccccc1)c1ccccc1 ; Cc1cccc2c1-c1ccc(N(c3cc4ccccc4cc3-c3ccccc3C(C)(C)C)C3CCC(c4ccccc4)CC3)cc1C2(c1ccccc1)c1ccccc1] 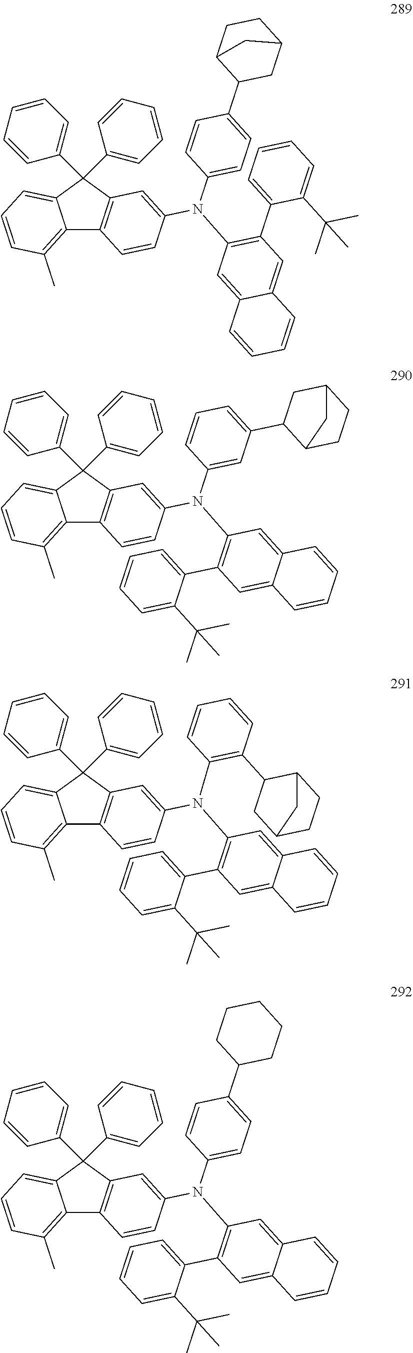 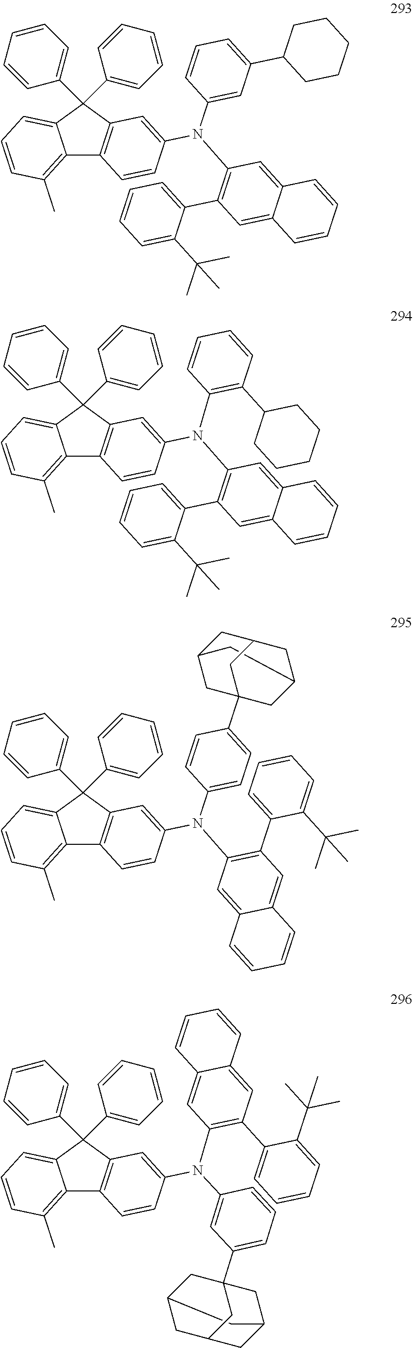 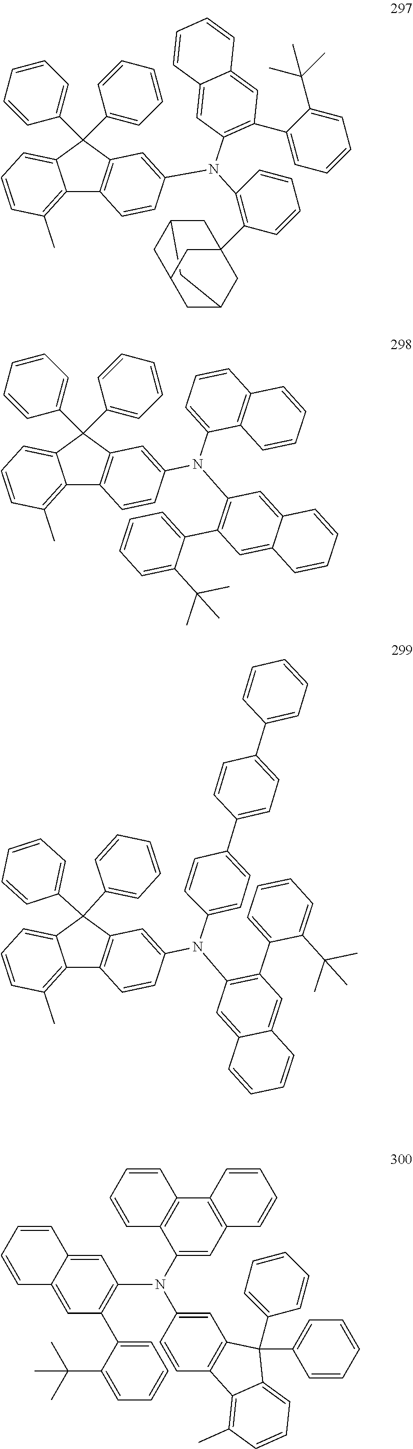 [SMILES: Cc1cccc2c1-c1ccc(N(c3cc4ccccc4cc3-c3ccccc3C(C)(C)C)c3ccccc3C34CC5CC(CC(C5)C3)C4)cc1C2(c1ccccc1)c1ccccc1 ; Cc1cccc2c1-c1ccc(N(c3cc4ccccc4cc3-c3ccccc3C(C)(C)C)c3cccc4ccccc34)cc1C2(c1ccccc1)c1ccccc1 ; Cc1cccc2c1-c1ccc(N(c3ccc(-c4ccc(-c5ccccc5)cc4)cc3)c3cc4ccccc4cc3-c3ccccc3C(C)(C)C)cc1C2(c1ccccc1)c1ccccc1 ; Cc1cccc2c1-c1ccc(N(c3cc4ccccc4cc3-c3ccccc3C(C)(C)C)c3cc4ccccc4c4ccccc34)cc1C2(c1ccccc1)c1ccccc1] 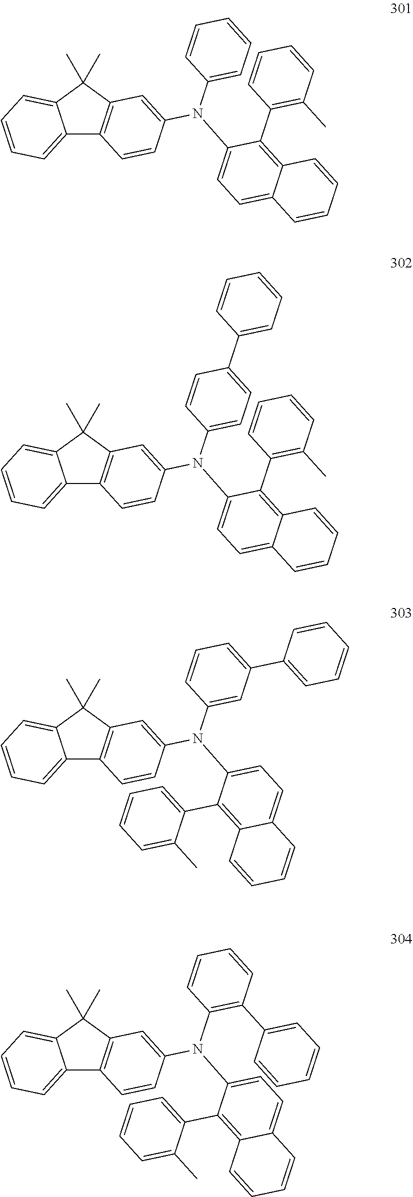 [SMILES: Cc1ccccc1-c1c(N(c2ccccc2)c2ccc3c(c2)C(C)(C)c2ccccc2-3)ccc2ccccc12 ; Cc1ccccc1-c1c(N(c2ccc(-c3ccccc3)cc2)c2ccc3c(c2)C(C)(C)c2ccccc2-3)ccc2ccccc12 ; Cc1ccccc1-c1c(N(c2cccc(-c3ccccc3)c2)c2ccc3c(c2)C(C)(C)c2ccccc2-3)ccc2ccccc12 ; Cc1ccccc1-c1c(N(c2ccc3c(c2)C(C)(C)c2ccccc2-3)c2ccccc2-c2ccccc2)ccc2ccccc12] 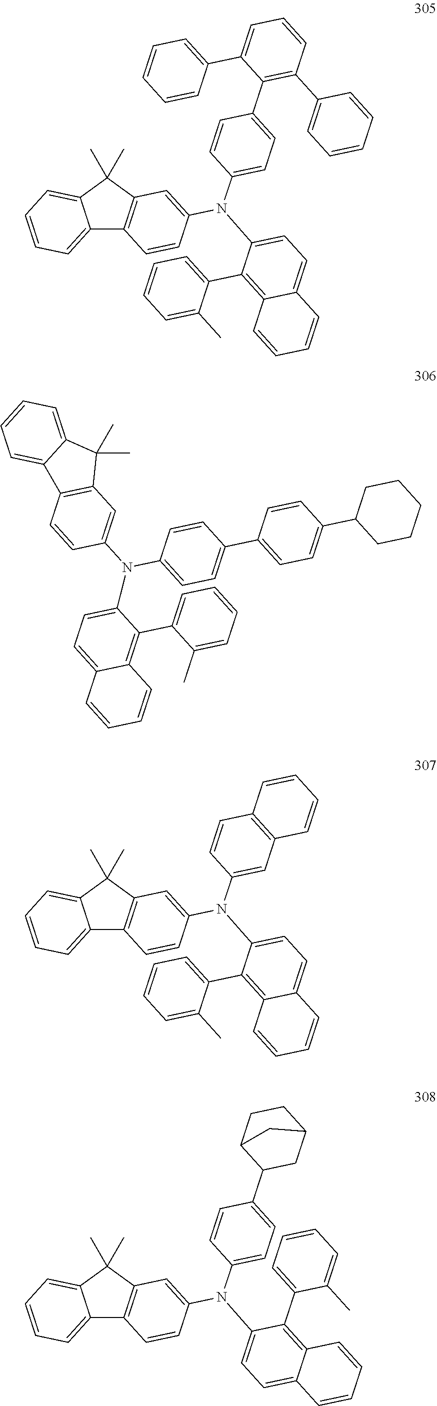 [SMILES: Cc1ccccc1-c1c(N(c2ccc(-c3c(-c4ccccc4)cccc3-c3ccccc3)cc2)c2ccc3c(c2)C(C)(C)c2ccccc2-3)ccc2ccccc12 ; Cc1ccccc1-c1c(N(c2ccc(-c3ccc(C4CCCCC4)cc3)cc2)c2ccc3c(c2)C(C)(C)c2ccccc2-3)ccc2ccccc12 ; Cc1ccccc1-c1c(N(c2ccc3c(c2)C(C)(C)c2ccccc2-3)c2ccc3ccccc3c2)ccc2ccccc12 ; Cc1ccccc1-c1c(N(c2ccc(C3CC4CCC3C4)cc2)c2ccc3c(c2)C(C)(C)c2ccccc2-3)ccc2ccccc12] 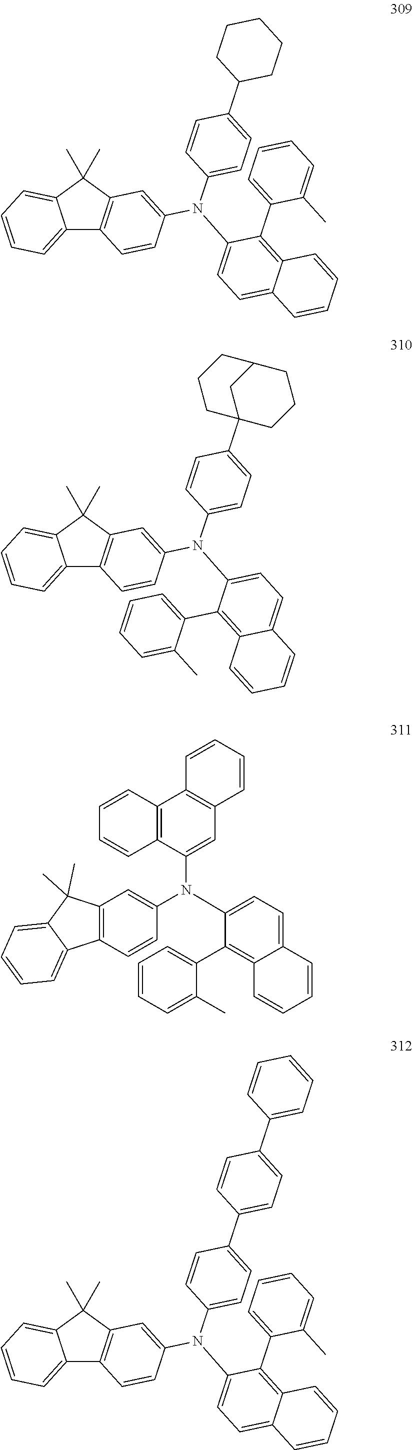 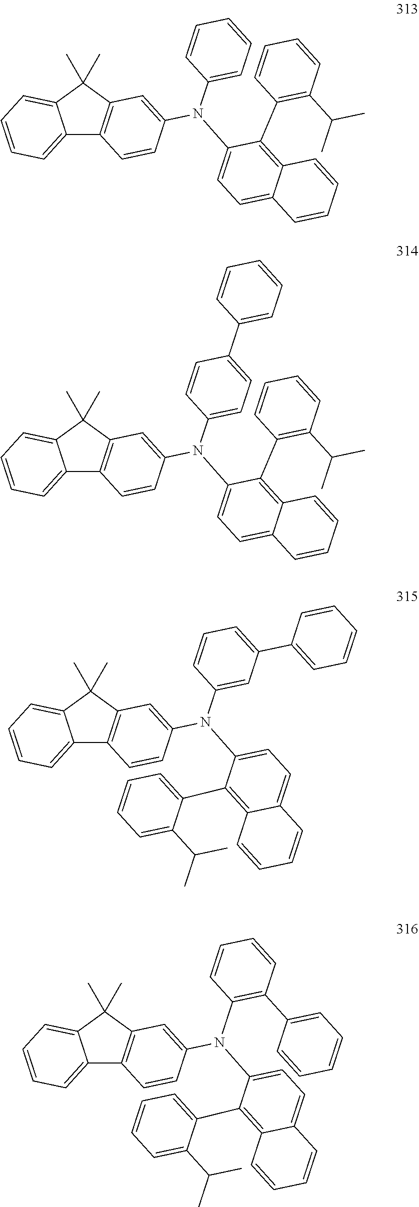 [SMILES: CC(C)c1ccccc1-c1c(N(c2ccccc2)c2ccc3c(c2)C(C)(C)c2ccccc2-3)ccc2ccccc12 ; CC(C)c1ccccc1-c1c(N(c2ccc(-c3ccccc3)cc2)c2ccc3c(c2)C(C)(C)c2ccccc2-3)ccc2ccccc12 ; CC(C)c1ccccc1-c1c(N(c2cccc(-c3ccccc3)c2)c2ccc3c(c2)C(C)(C)c2ccccc2-3)ccc2ccccc12 ; CC(C)c1ccccc1-c1c(N(c2ccc3c(c2)C(C)(C)c2ccccc2-3)c2ccccc2-c2ccccc2)ccc2ccccc12] 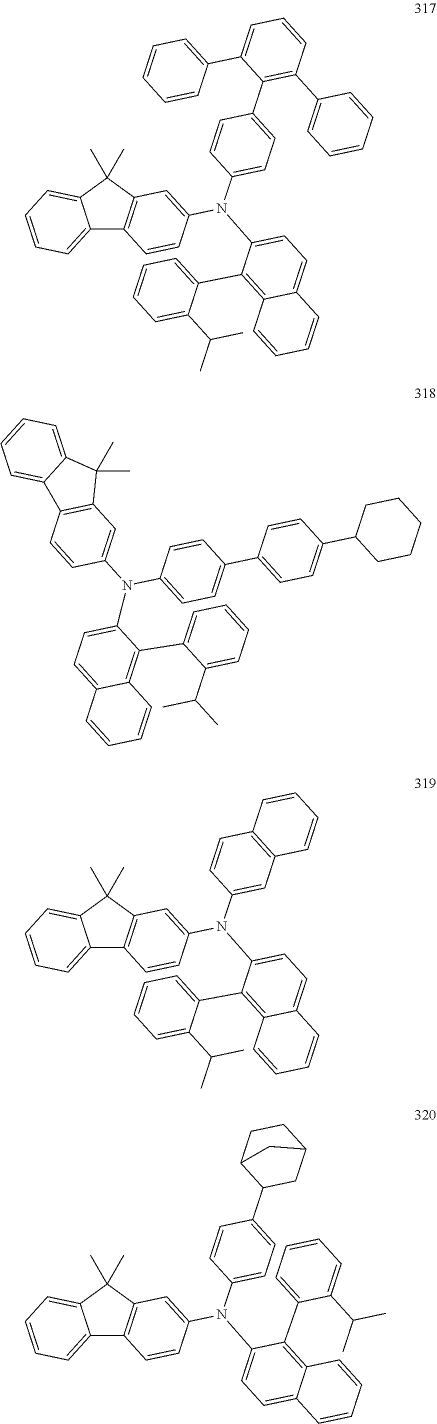 [SMILES: CC(C)c1ccccc1-c1c(N(c2ccc(-c3c(-c4ccccc4)cccc3-c3ccccc3)cc2)c2ccc3c(c2)C(C)(C)c2ccccc2-3)ccc2ccccc12 ; CC(C)c1ccccc1-c1c(N(c2ccc(-c3ccc(C4CCCCC4)cc3)cc2)c2ccc3c(c2)C(C)(C)c2ccccc2-3)ccc2ccccc12 ; CC(C)c1ccccc1-c1c(N(c2ccc3c(c2)C(C)(C)c2ccccc2-3)c2ccc3ccccc3c2)ccc2ccccc12 ; CC(C)c1ccccc1-c1c(N(c2ccc(C3CC4CCC3C4)cc2)c2ccc3c(c2)C(C)(C)c2ccccc2-3)ccc2ccccc12] 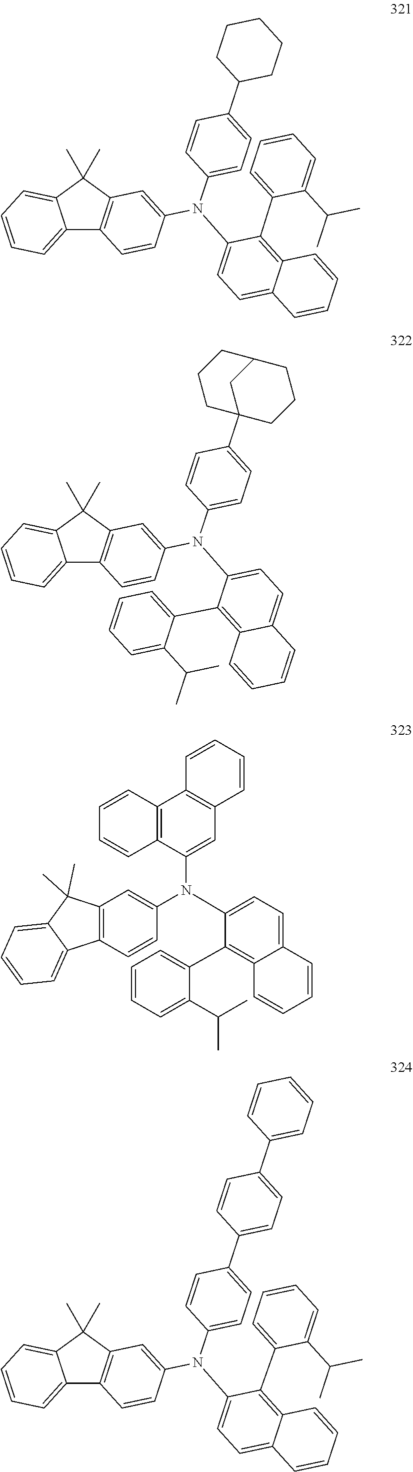 [SMILES: CC(C)c1ccccc1-c1c(N(c2ccc(C3CCCCC3)cc2)c2ccc3c(c2)C(C)(C)c2ccccc2-3)ccc2ccccc12 ; CC(C)c1ccccc1-c1c(N(c2ccc(C34CCCC(CCC3)C4)cc2)c2ccc3c(c2)C(C)(C)c2ccccc2-3)ccc2ccccc12 ; CC(C)c1ccccc1-c1c(N(c2ccc3c(c2)C(C)(C)c2ccccc2-3)c2cc3ccccc3c3ccccc23)ccc2ccccc12 ; CC(C)c1ccccc1-c1c(N(c2ccc(-c3ccc(-c4ccccc4)cc3)cc2)c2ccc3c(c2)C(C)(C)c2ccccc2-3)ccc2ccccc12] 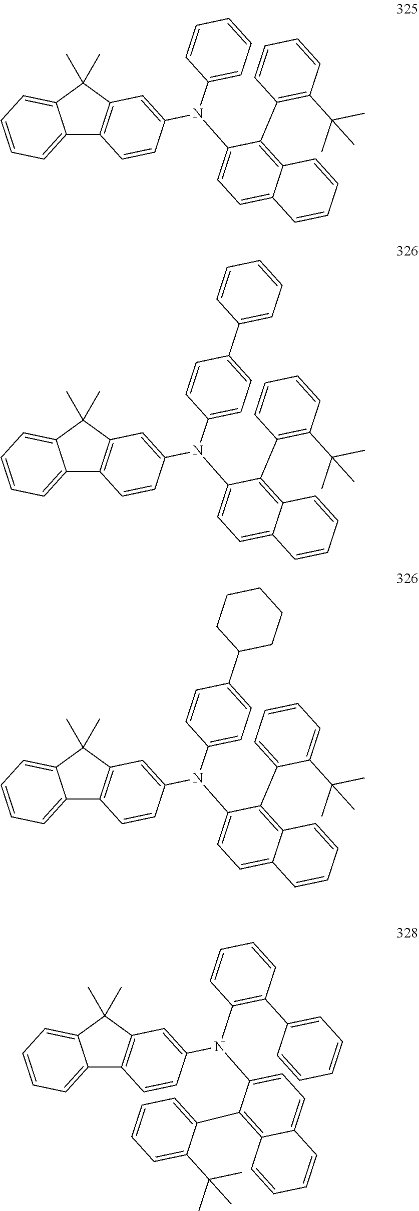 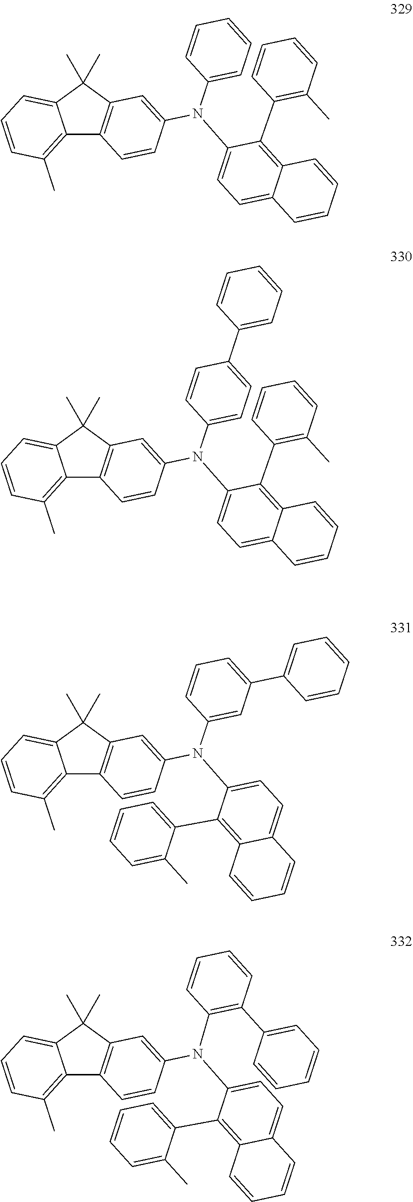 [SMILES: Cc1ccccc1-c1c(N(c2ccccc2)c2ccc3c(c2)C(C)(C)c2cccc(C)c2-3)ccc2ccccc12 ; Cc1ccccc1-c1c(N(c2ccc(-c3ccccc3)cc2)c2ccc3c(c2)C(C)(C)c2cccc(C)c2-3)ccc2ccccc12 ; Cc1ccccc1-c1c(N(c2cccc(-c3ccccc3)c2)c2ccc3c(c2)C(C)(C)c2cccc(C)c2-3)ccc2ccccc12 ; Cc1ccccc1-c1c(N(c2ccc3c(c2)C(C)(C)c2cccc(C)c2-3)c2ccccc2-c2ccccc2)ccc2ccccc12] 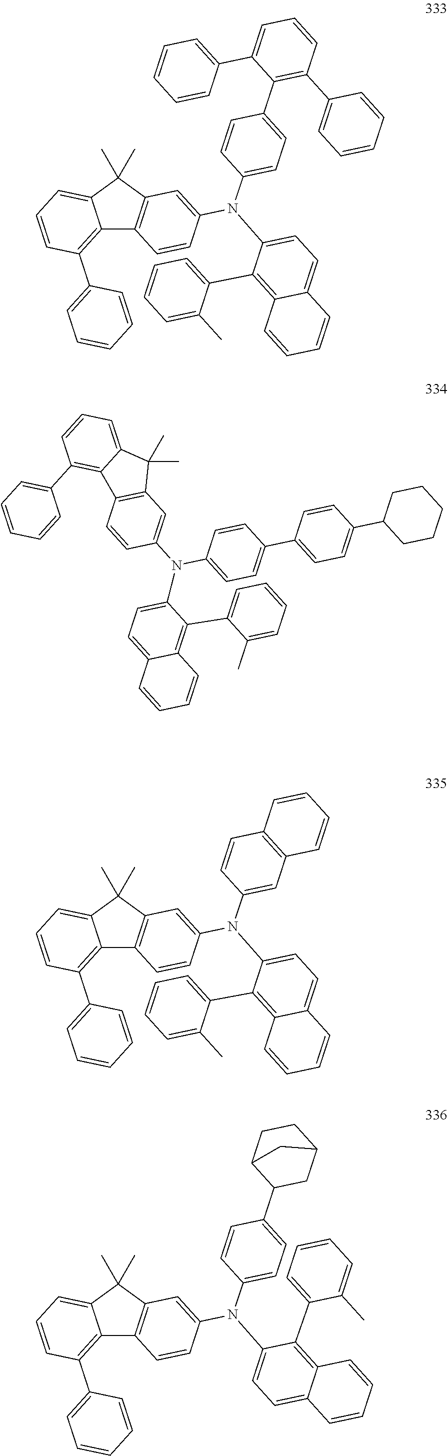 [SMILES: Cc1ccccc1-c1c(N(c2ccc(-c3c(-c4ccccc4)cccc3-c3ccccc3)cc2)c2ccc3c(c2)C(C)(C)c2cccc(-c4ccccc4)c2-3)ccc2ccccc12 ; Cc1ccccc1-c1c(N(c2ccc(-c3ccc(C4CCCCC4)cc3)cc2)c2ccc3c(c2)C(C)(C)c2cccc(-c4ccccc4)c2-3)ccc2ccccc12 ; Cc1ccccc1-c1c(N(c2ccc3c(c2)C(C)(C)c2cccc(-c4ccccc4)c2-3)c2ccc3ccccc3c2)ccc2ccccc12 ; Cc1ccccc1-c1c(N(c2ccc(C3CC4CCC3C4)cc2)c2ccc3c(c2)C(C)(C)c2cccc(-c4ccccc4)c2-3)ccc2ccccc12] 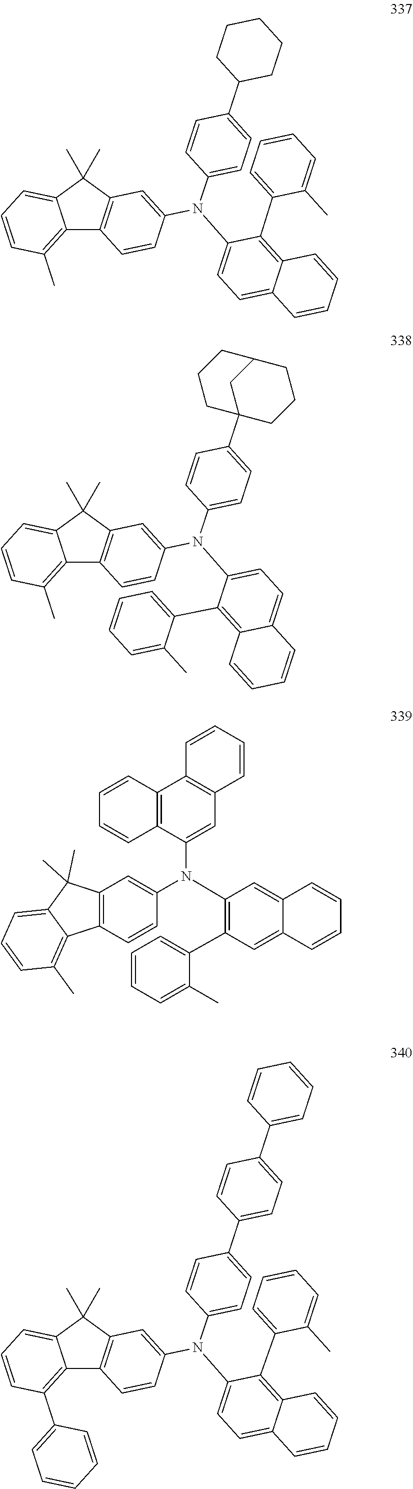 [SMILES: Cc1ccccc1-c1c(N(c2ccc(C3CCCCC3)cc2)c2ccc3c(c2)C(C)(C)c2cccc(C)c2-3)ccc2ccccc12 ; Cc1ccccc1-c1c(N(c2ccc(C34CCCC(CCC3)C4)cc2)c2ccc3c(c2)C(C)(C)c2cccc(C)c2-3)ccc2ccccc12 ; Cc1ccccc1-c1cc2ccccc2cc1N(c1ccc2c(c1)C(C)(C)c1cccc(C)c1-2)c1cc2ccccc2c2ccccc12 ; Cc1ccccc1-c1c(N(c2ccc(-c3ccc(-c4ccccc4)cc3)cc2)c2ccc3c(c2)C(C)(C)c2cccc(-c4ccccc4)c2-3)ccc2ccccc12] 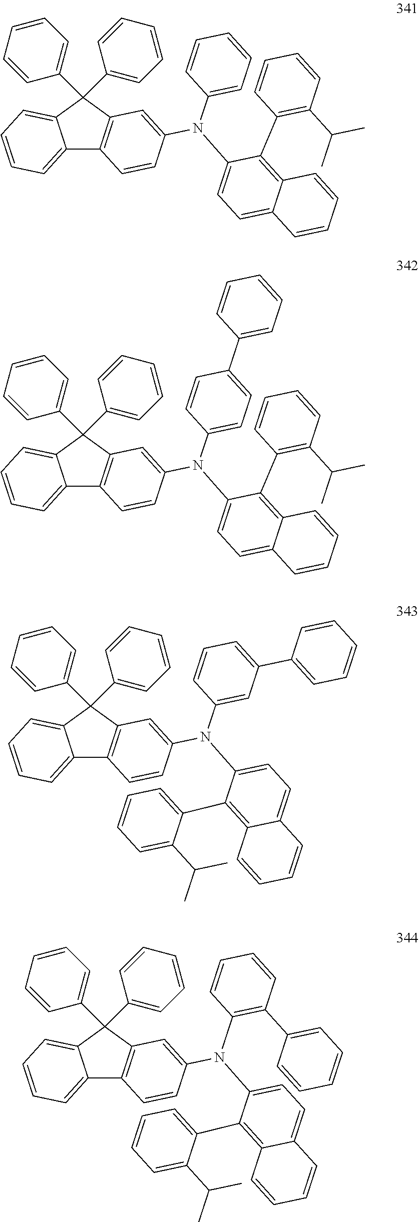 [SMILES: CC(C)c1ccccc1-c1c(N(c2ccccc2)c2ccc3c(c2)C(c2ccccc2)(c2ccccc2)c2ccccc2-3)ccc2ccccc12 ; CC(C)c1ccccc1-c1c(N(c2ccc(-c3ccccc3)cc2)c2ccc3c(c2)C(c2ccccc2)(c2ccccc2)c2ccccc2-3)ccc2ccccc12 ; CC(C)c1ccccc1-c1c(N(c2cccc(-c3ccccc3)c2)c2ccc3c(c2)C(c2ccccc2)(c2ccccc2)c2ccccc2-3)ccc2ccccc12 ; CC(C)c1ccccc1-c1c(N(c2ccc3c(c2)C(c2ccccc2)(c2ccccc2)c2ccccc2-3)c2ccccc2-c2ccccc2)ccc2ccccc12] 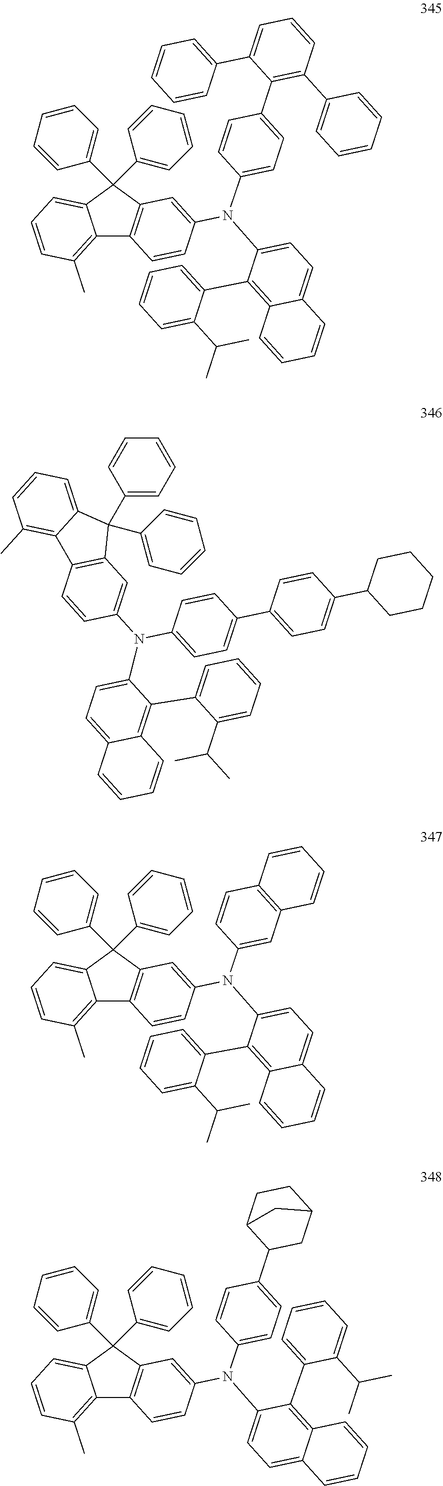 [SMILES: Cc1cccc2c1-c1ccc(N(c3ccc(-c4c(-c5ccccc5)cccc4-c4ccccc4)cc3)c3ccc4ccccc4c3-c3ccccc3C(C)C)cc1C2(c1ccccc1)c1ccccc1 ; Cc1cccc2c1-c1ccc(N(c3ccc(-c4ccc(C5CCCCC5)cc4)cc3)c3ccc4ccccc4c3-c3ccccc3C(C)C)cc1C2(c1ccccc1)c1ccccc1 ; Cc1cccc2c1-c1ccc(N(c3ccc4ccccc4c3)c3ccc4ccccc4c3-c3ccccc3C(C)C)cc1C2(c1ccccc1)c1ccccc1 ; Cc1cccc2c1-c1ccc(N(c3ccc(C4CC5CCC4C5)cc3)c3ccc4ccccc4c3-c3ccccc3C(C)C)cc1C2(c1ccccc1)c1ccccc1] 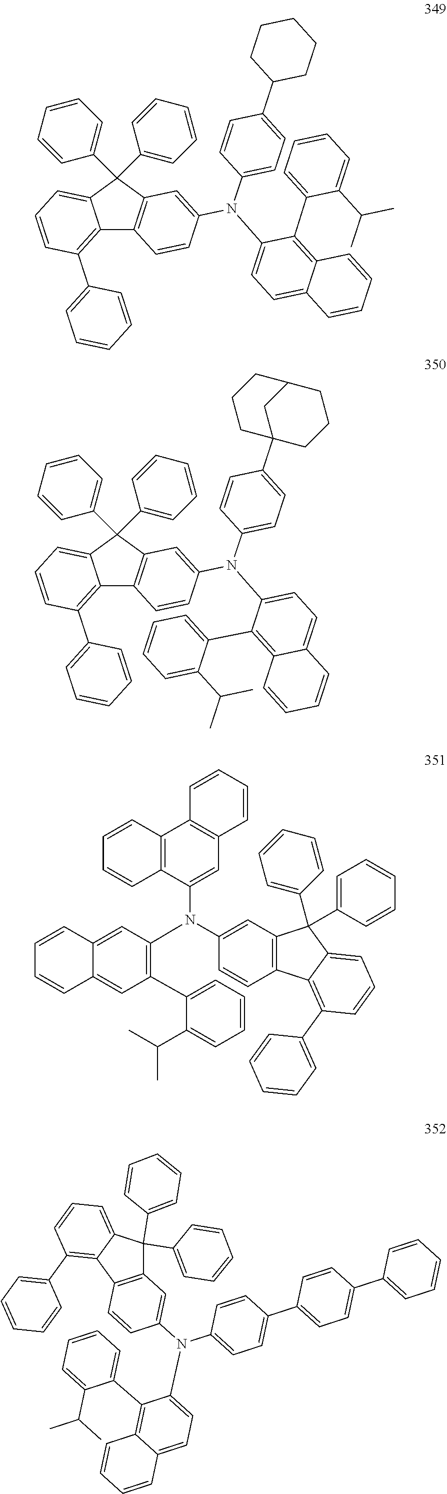 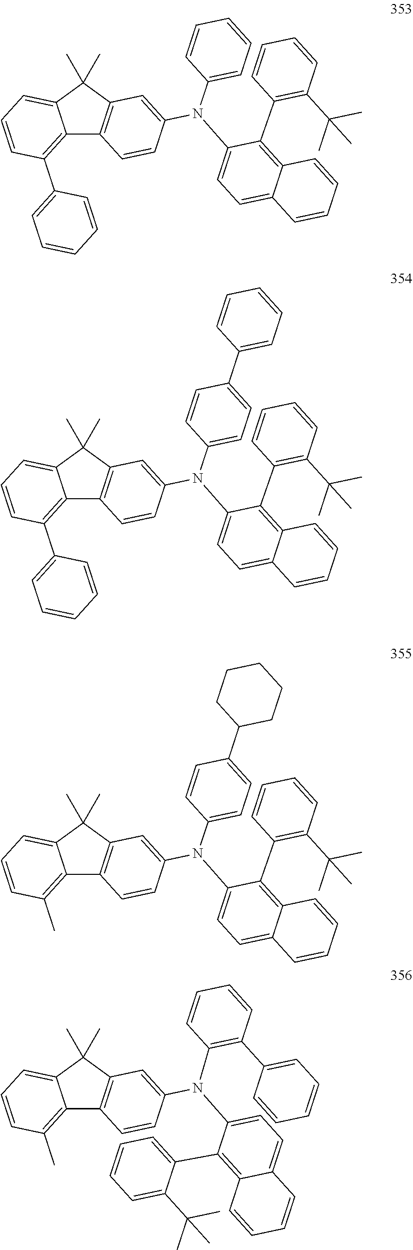 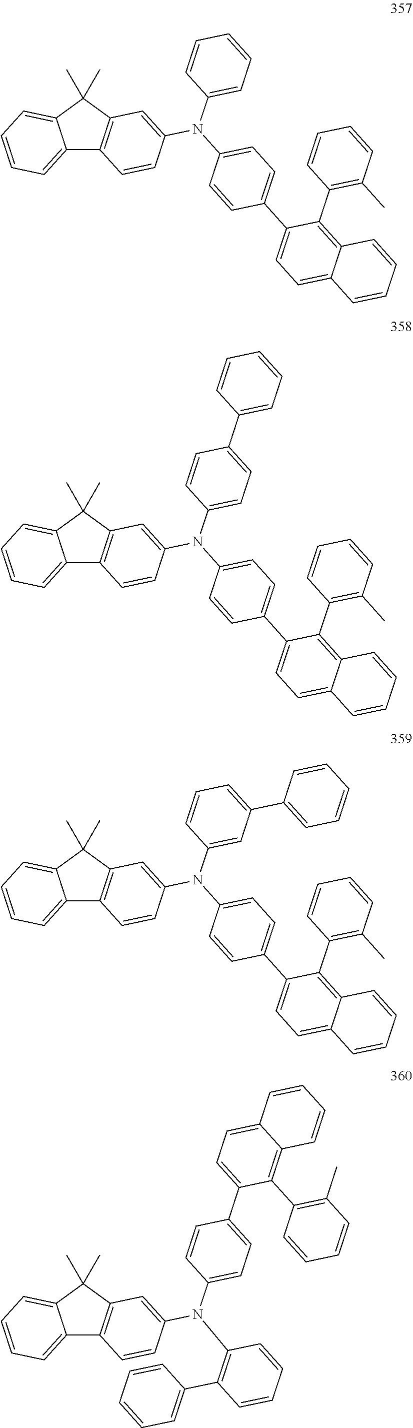 [SMILES: Cc1ccccc1-c1c(-c2ccc(N(c3ccccc3)c3ccc4c(c3)C(C)(C)c3ccccc3-4)cc2)ccc2ccccc12 ; Cc1ccccc1-c1c(-c2ccc(N(c3ccc(-c4ccccc4)cc3)c3ccc4c(c3)C(C)(C)c3ccccc3-4)cc2)ccc2ccccc12 ; Cc1ccccc1-c1c(-c2ccc(N(c3cccc(-c4ccccc4)c3)c3ccc4c(c3)C(C)(C)c3ccccc3-4)cc2)ccc2ccccc12 ; Cc1ccccc1-c1c(-c2ccc(N(c3ccc4c(c3)C(C)(C)c3ccccc3-4)c3ccccc3-c3ccccc3)cc2)ccc2ccccc12] 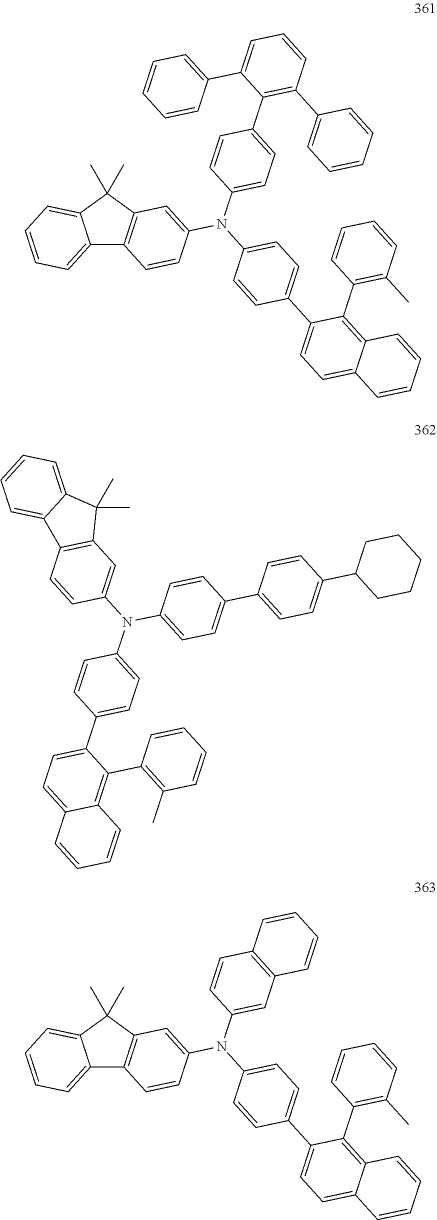 [SMILES: Cc1ccccc1-c1c(-c2ccc(N(c3ccc(-c4c(-c5ccccc5)cccc4-c4ccccc4)cc3)c3ccc4c(c3)C(C)(C)c3ccccc3-4)cc2)ccc2ccccc12 ; Cc1ccccc1-c1c(-c2ccc(N(c3ccc(-c4ccc(C5CCCCC5)cc4)cc3)c3ccc4c(c3)C(C)(C)c3ccccc3-4)cc2)ccc2ccccc12 ; Cc1ccccc1-c1c(-c2ccc(N(c3ccc4c(c3)C(C)(C)c3ccccc3-4)c3ccc4ccccc4c3)cc2)ccc2ccccc12] 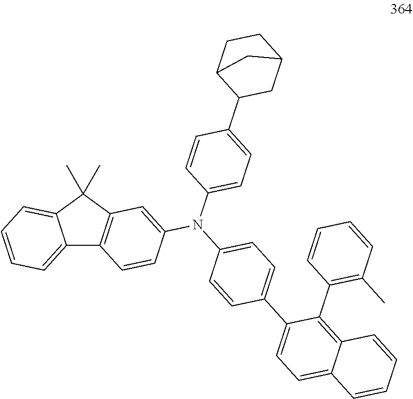 [SMILES: Cc1ccccc1-c1c(-c2ccc(N(c3ccc(C4CC5CCC4C5)cc3)c3ccc4c(c3)C(C)(C)c3ccccc3-4)cc2)ccc2ccccc12] 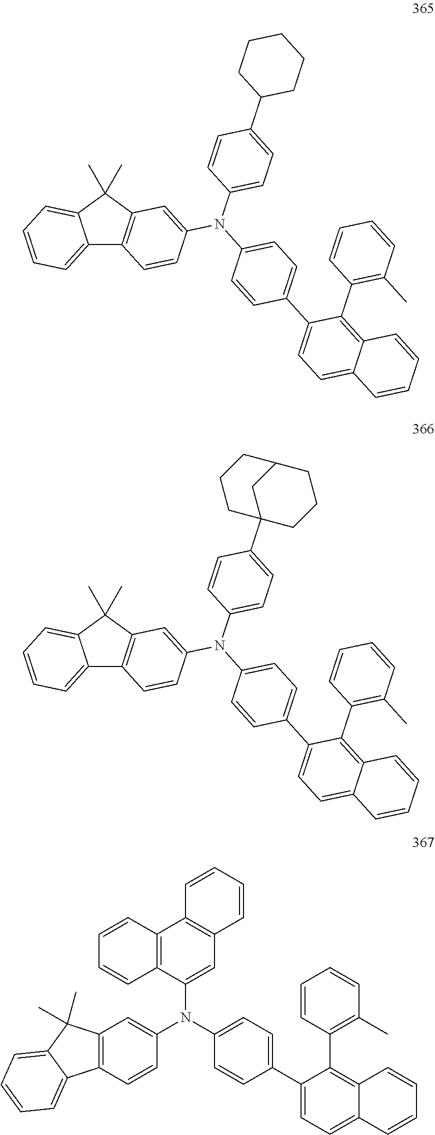 [SMILES: Cc1ccccc1-c1c(-c2ccc(N(c3ccc(C4CCCCC4)cc3)c3ccc4c(c3)C(C)(C)c3ccccc3-4)cc2)ccc2ccccc12 ; Cc1ccccc1-c1c(-c2ccc(N(c3ccc(C45CCCC(CCC4)C5)cc3)c3ccc4c(c3)C(C)(C)c3ccccc3-4)cc2)ccc2ccccc12 ; Cc1ccccc1-c1c(-c2ccc(N(c3ccc4c(c3)C(C)(C)c3ccccc3-4)c3cc4ccccc4c4ccccc34)cc2)ccc2ccccc12] 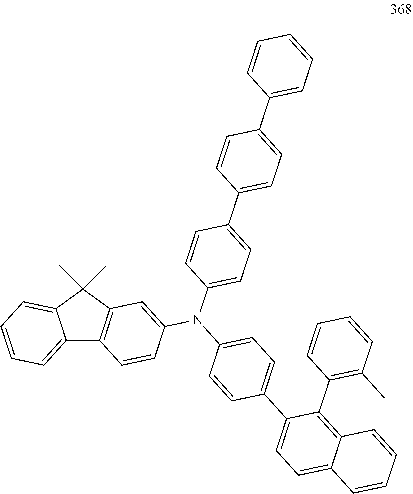 [SMILES: Cc1ccccc1-c1c(-c2ccc(N(c3ccc(-c4ccc(-c5ccccc5)cc4)cc3)c3ccc4c(c3)C(C)(C)c3ccccc3-4)cc2)ccc2ccccc12] 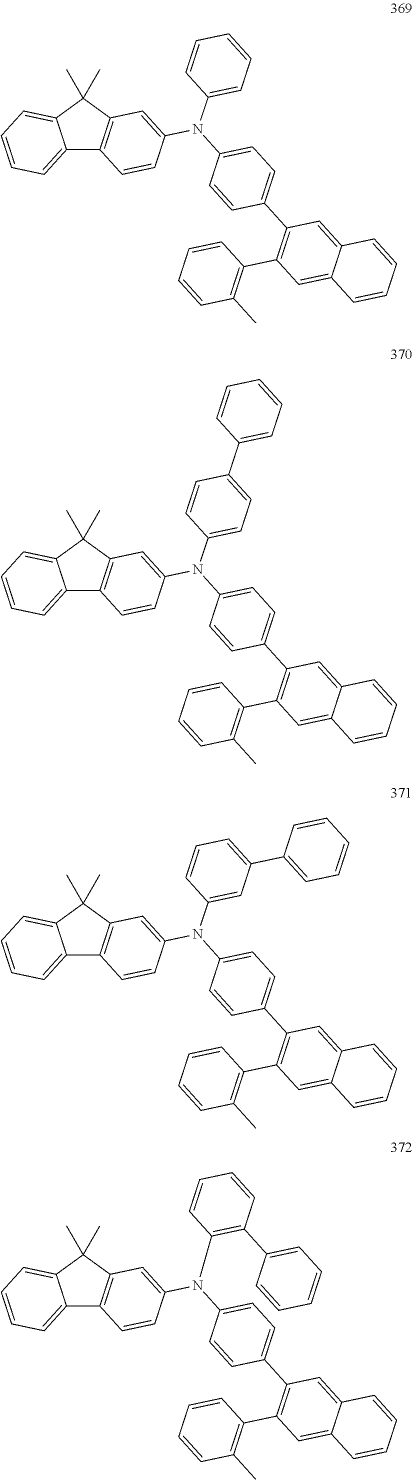 [SMILES: Cc1ccccc1-c1cc2ccccc2cc1-c1ccc(N(c2ccccc2)c2ccc3c(c2)C(C)(C)c2ccccc2-3)cc1 ; Cc1ccccc1-c1cc2ccccc2cc1-c1ccc(N(c2ccc(-c3ccccc3)cc2)c2ccc3c(c2)C(C)(C)c2ccccc2-3)cc1 ; Cc1ccccc1-c1cc2ccccc2cc1-c1ccc(N(c2cccc(-c3ccccc3)c2)c2ccc3c(c2)C(C)(C)c2ccccc2-3)cc1 ; Cc1ccccc1-c1cc2ccccc2cc1-c1ccc(N(c2ccc3c(c2)C(C)(C)c2ccccc2-3)c2ccccc2-c2ccccc2)cc1] 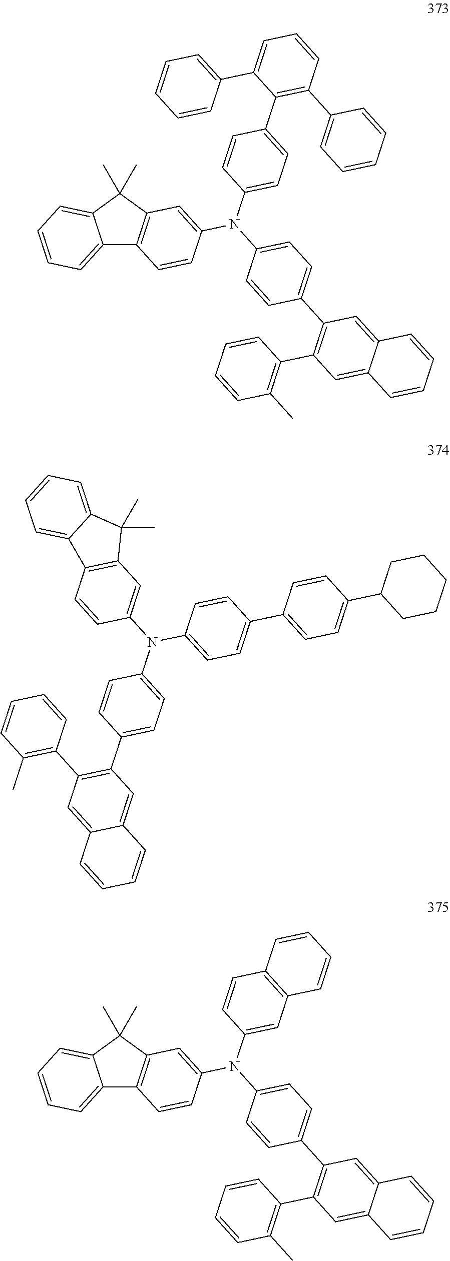 [SMILES: Cc1ccccc1-c1cc2ccccc2cc1-c1ccc(N(c2ccc(-c3c(-c4ccccc4)cccc3-c3ccccc3)cc2)c2ccc3c(c2)C(C)(C)c2ccccc2-3)cc1 ; Cc1ccccc1-c1cc2ccccc2cc1-c1ccc(N(c2ccc(-c3ccc(C4CCCCC4)cc3)cc2)c2ccc3c(c2)C(C)(C)c2ccccc2-3)cc1 ; Cc1ccccc1-c1cc2ccccc2cc1-c1ccc(N(c2ccc3c(c2)C(C)(C)c2ccccc2-3)c2ccc3ccccc3c2)cc1] 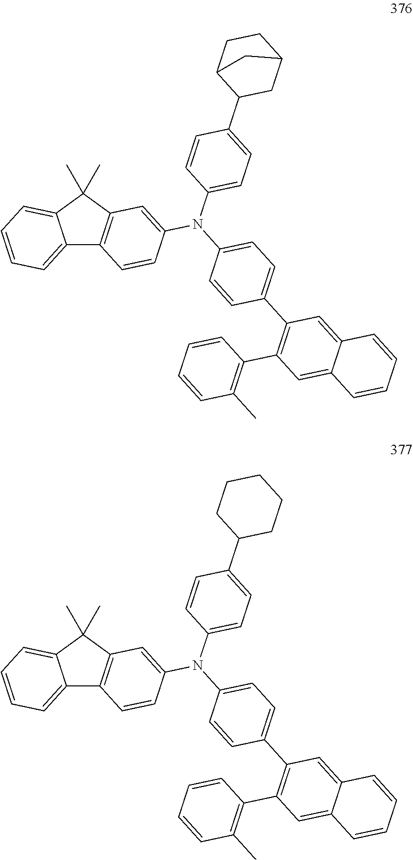 [SMILES: Cc1ccccc1-c1cc2ccccc2cc1-c1ccc(N(c2ccc(C3CC4CCC3C4)cc2)c2ccc3c(c2)C(C)(C)c2ccccc2-3)cc1 ; Cc1ccccc1-c1cc2ccccc2cc1-c1ccc(N(c2ccc(C3CCCCC3)cc2)c2ccc3c(c2)C(C)(C)c2ccccc2-3)cc1] 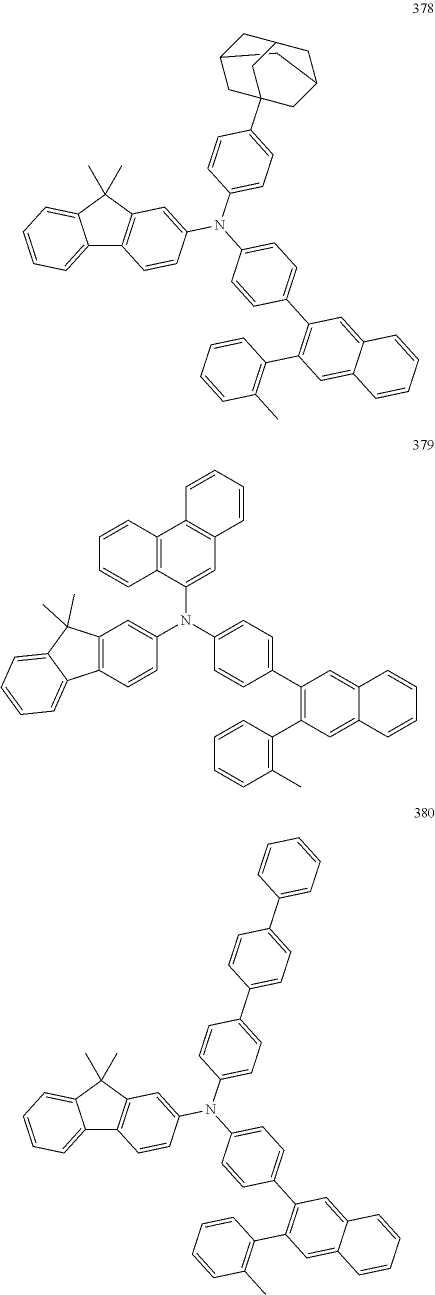 [SMILES: Cc1ccccc1-c1cc2ccccc2cc1-c1ccc(N(c2ccc(C34CC5CC(CC(C5)C3)C4)cc2)c2ccc3c(c2)C(C)(C)c2ccccc2-3)cc1 ; Cc1ccccc1-c1cc2ccccc2cc1-c1ccc(N(c2ccc3c(c2)C(C)(C)c2ccccc2-3)c2cc3ccccc3c3ccccc23)cc1 ; Cc1ccccc1-c1cc2ccccc2cc1-c1ccc(N(c2ccc(-c3ccc(-c4ccccc4)cc3)cc2)c2ccc3c(c2)C(C)(C)c2ccccc2-3)cc1] 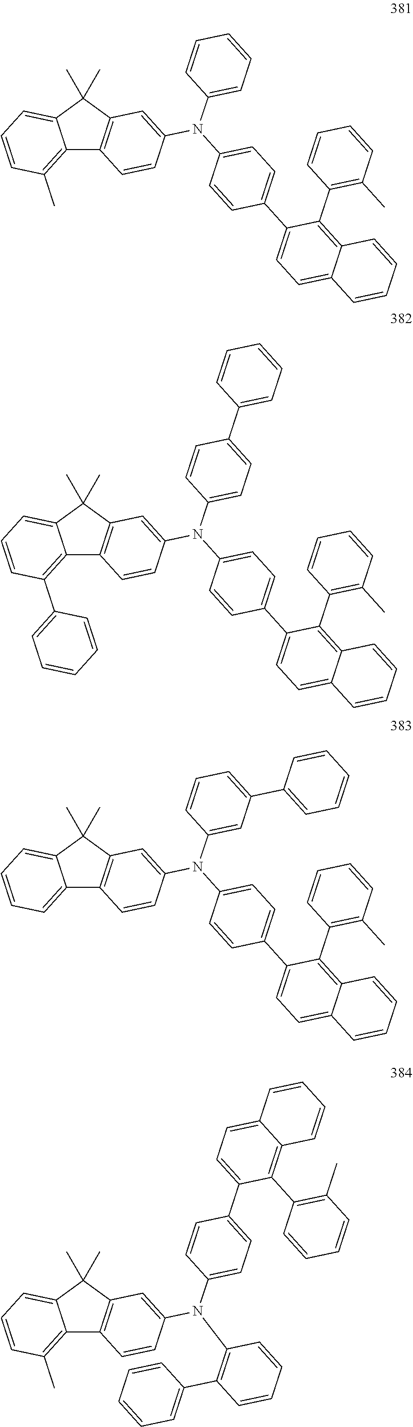 [SMILES: Cc1ccccc1-c1c(-c2ccc(N(c3ccccc3)c3ccc4c(c3)C(C)(C)c3cccc(C)c3-4)cc2)ccc2ccccc12 ; Cc1ccccc1-c1c(-c2ccc(N(c3ccc(-c4ccccc4)cc3)c3ccc4c(c3)C(C)(C)c3cccc(-c5ccccc5)c3-4)cc2)ccc2ccccc12 ; Cc1ccccc1-c1c(-c2ccc(N(c3cccc(-c4ccccc4)c3)c3ccc4c(c3)C(C)(C)c3ccccc3-4)cc2)ccc2ccccc12 ; Cc1ccccc1-c1c(-c2ccc(N(c3ccc4c(c3)C(C)(C)c3cccc(C)c3-4)c3ccccc3-c3ccccc3)cc2)ccc2ccccc12] 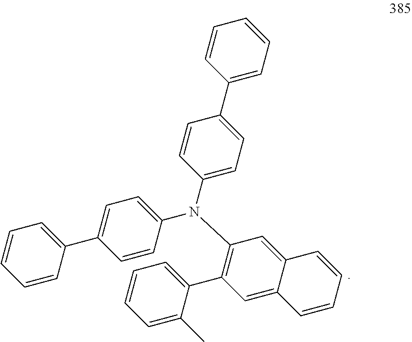 [SMILES: Cc1ccccc1-c1cc2ccccc2cc1N(c1ccc(-c2ccccc2)cc1)c1ccc(-c2ccccc2)cc1]
